(19) United States
(12) Patent Application Publication
(10) Pub. No.: US 2025/0312367 A1
(43) Pub. Date: Oct. 9, 2025

(54) OLIGONUCLEOTIDES HAVING A SYNTHETIC BACKBONE AND SYNTHESIS THEREOF

(71) Applicant: ADARx Pharmaceuticals, Inc., San Diego, CA (US)

(72) Inventors: Zhen Li, San Diego, CA (US); Chandramouli Chiruta, San Diego, CA (US); Bo Cheng, San Diego, CA (US); Mehdi Michel Djamel Numa, San Diego, CA (US); Mihai Azimioara, San Diego, CA (US); Chase Robert Olsson, San Diego, CA (US); Rui Zhu, San diego, CA (US); Kin Fong, San Diego, CA (US)

(73) Assignee: ADARx Pharmaceuticals, Inc., San diego, CA (US)

(21) Appl. No.: 18/865,220

(22) PCT Filed: May 12, 2023
(86) PCT No.: PCT/US2023/066961

§ 371 Date: Nov. 12, 2024

Related U.S. Application Data
(60) Provisional application No. 63/342,018, filed on May 13, 2022.

(51) Int. Cl. A61K 31/702; A61K 31/7016; C07H 21/04

(52) U.S. Cl. CPC A61K 31/702; A61K 31/7016; C07H 21/04

(57) ABSTRACT

Provided herein are oligonucleotide-containing compounds, methods of delivering the compounds, and methods of treating diseases, disorders, and symptoms (e.g., central nervous system diseases, disorders, and symptoms) in a subject using the compounds.

### CROSS-REFERENCE TO RELATED APPLICATIONS

This application claims the benefit of priority under 35 U.S.C. § 119(e) of U.S. Provisional Application No. 63/342,018, filed May 13, 2022. The disclosure of the prior application is considered part of and is incorporated by reference in its entirety into the disclosure of this application.

### BACKGROUND

In the use of compounds in therapeutic, prophylactic, or diagnostic applications, it is often desirable that the compounds be delivered to a specific location (for example, to desired cell(s)) to enhance the therapeutic or prophylactic effect or to be advantageous for diagnostic purposes. This is frequently the case when attempting to deliver a therapeutic compound in vivo. Further, being able to efficiently deliver a compound to a specific location can limit or potentially eliminate unintended consequences (such as off-target effects) that may be caused by administration of the compound. One strategy to facilitate delivery of a compound, such as a therapeutic, prophylactic, or diagnostic compound, to a desired location in vivo, is by linking or attaching the compound to a targeting ligand.

One class of compounds that can be targeted using targeting ligands are oligomeric compounds, such as, for example, proteins, peptides, antibodies, and oligonucleotides. Oligomeric compounds that include nucleotide sequences (e.g., oligonucleotides) at least partially complementary to a target nucleic acid have been shown to alter the function and activity of the target both in vitro and in vivo. When delivered to a cell containing a target nucleic acid (such as mRNA or pre-mRNA), oligonucleotides have been shown to modulate the expression or activity of the target nucleic acid. In certain instances, the oligonucleotide can reduce the expression of the gene by inhibiting translation of the nucleic acid target and/or triggering the degradation of the target nucleic acid.

If the target nucleic acid is mRNA, one mechanism by which an oligonucleotide can modulate the expression of the mRNA target is through RNA interference. RNA interference is a biological process by which RNA or RNA-like molecules (such as chemically modified RNA molecules) are able to silence gene expression, at least in part, through the RNA-Induced Silencing Complex (RISC) pathway. Additionally, oligonucleotides can modulate the expression of a target nucleic acid, such as a target mRNA, through an RNase recruitment mechanism, microRNA mechanisms, occupancy-based mechanisms, and editing mechanisms. Oligonucleotides may be single-stranded or double-stranded. Oligonucleotides may comprise DNA, RNA, and RNA-like molecules, which can also include modified nucleosides including one or more non-phosphodiester linkages.

Another class of compounds that can be targeted using targeting ligands are small molecule compounds. The small molecule compounds (e.g., an organic compound having a molecular weight of ca. 1000 daltons or less) are typically shown to alter the function and/or activity of the target such that disease and/or disease symptoms are modulated or ameliorated, or are typically useful as a diagnostic marker when localized to the target. More efficient delivery of a compound to a specific location can limit or potentially eliminate unintended consequences (such as off-target effects) that may be caused by administration of the compound and provide improved localization of a diagnostic compound.

### SUMMARY

This disclosure is directed towards compounds (e.g., any of those delineated herein), modified oligonucleotides, and methods of modulating protein function and/or expression and treating diseases, disorders, and symptoms in a subject. The methods can comprise the compounds and modified oligonucleotides disclosed herein.

It is understood that the embodiments of the invention discussed below with respect to the preferred variable selections can be taken alone or in combination with one or more embodiments, or preferred variable selections of the invention, as if each combination were explicitly listed herein.

In some aspects, the present disclosure provides oligonucleotides of the Formula (I′):

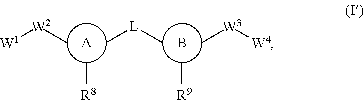

wherein:
A, B, W¹, W², W³, W⁴, R⁸, and R⁹ are as defined herein.

In some aspects, the present disclosure provides oligonucleotides of the Formula (VIII):

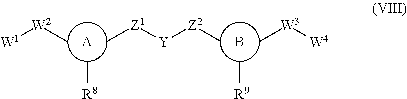

wherein:
A, B, W¹, W², W³, W⁴, R⁸, and R⁹ are as defined herein.

In some aspects, the present disclosure provides oligonucleotides of the Formula (I):

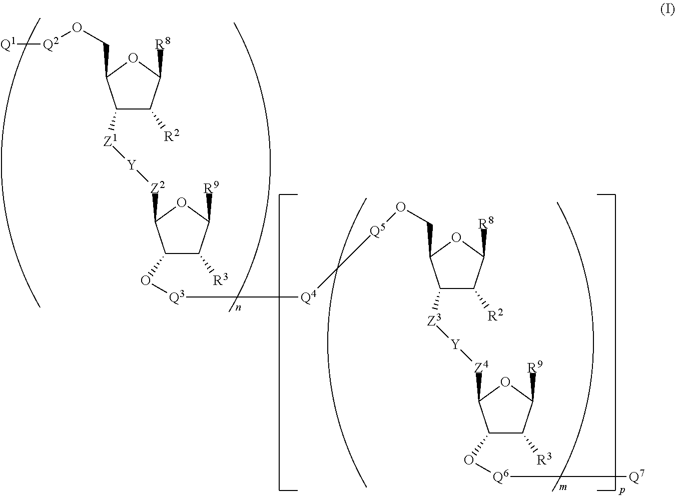

wherein:
Q¹, Q², Q³, Q⁴, Q⁵, Q⁶, Q⁷, Y, R², R³, R⁸, R⁹, Z¹, and Z² are as defined herein.

In some aspects, the present disclosure provides oligonucleotides of the Formula (VII):

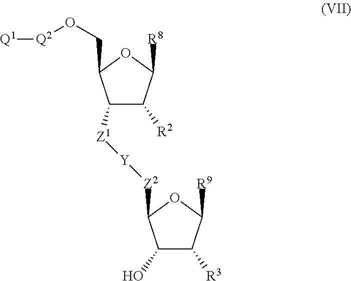

wherein:
Q¹, Q², Y, R², R³, R⁸, R⁹, Z¹, and Z² are as defined herein.

In some aspects, the present disclosure provides oligonucleotides of the Formula (II):

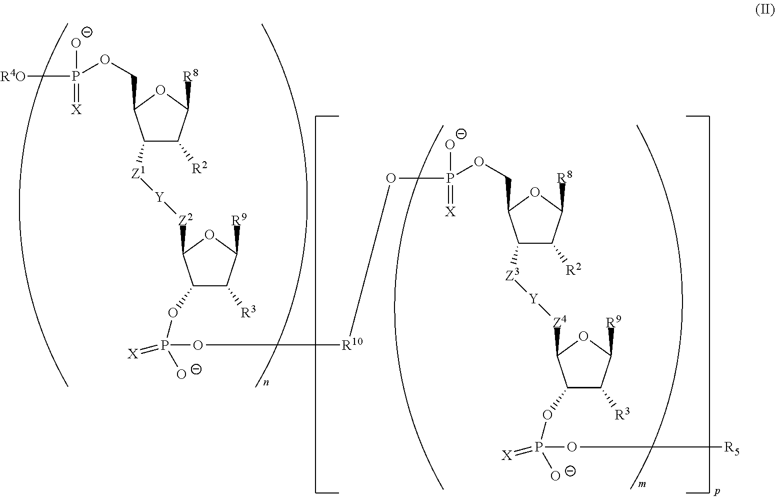

wherein:
X, Y, R², R³, R⁴, R⁵, R⁸, R⁹, Z¹, Z², Z³, Z⁴, n, and p are as defined herein.

In some aspects, the present disclosure provides oligonucleotides of the Formula (VI):

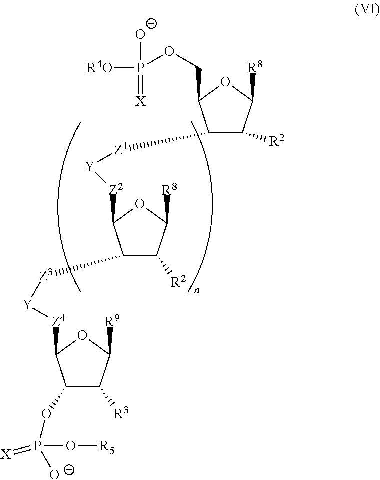

wherein:
X, Y, R², R³, R⁴, R⁵, R¹, R⁹, Z¹, Z², Z³, Z⁴, and n are as defined herein. In some aspects, the present disclosure provides oligonucleotides of the Formula (IX):

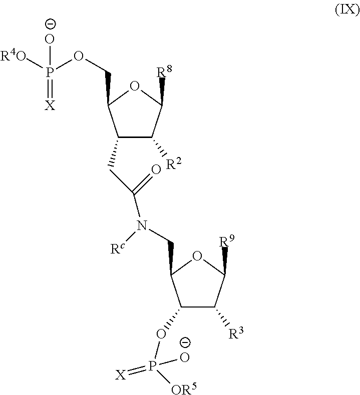

wherein:
X, R², R³, R⁴, R⁵, R⁸, R⁹, and R^{c} are as defined herein.

In some aspects, the present disclosure provides oligonucleotides of the Formula (X):

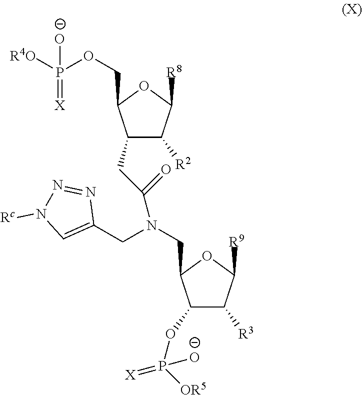

wherein:
X, R², R³, R⁴, R⁵, R⁸, R⁹, and R^{C} are as defined herein.

In some aspects, the present disclosure provides oligonucleotides of the Formula (XI):

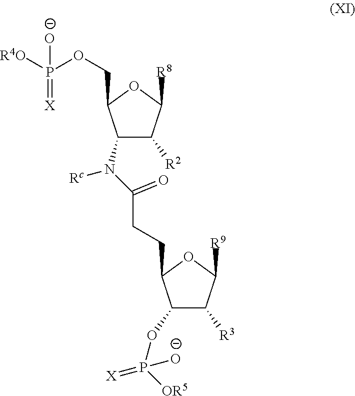

wherein:
X, R², R³, R⁴, R⁵, R⁸, R⁹, and R^{c} are as defined herein. In some aspects, the present disclosure provides oligonucleotides of the Formula (XII):

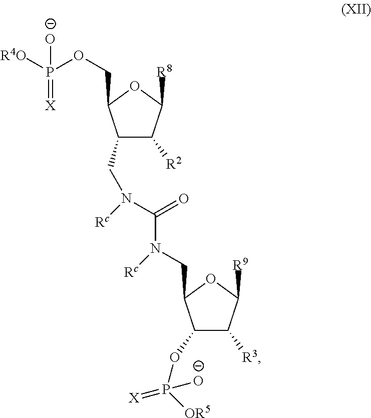

wherein:
X, R², R³, R⁴, R⁵, R⁸, R⁹, and R^{c} are as defined herein.

In some aspects, the present disclosure provides oligonucleotides of Formula (XIII):

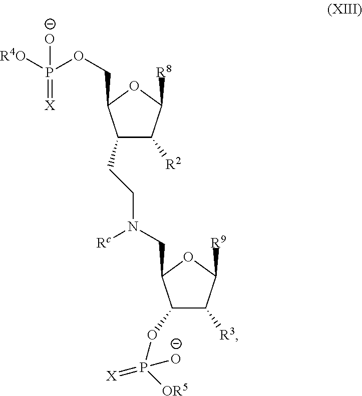

wherein:
X, R², R³, R⁴, R⁵, R⁸, R⁹, and R^{c} are as defined herein.

In some aspects, the present disclosure provides oligonucleotides of the Formula (XIV):

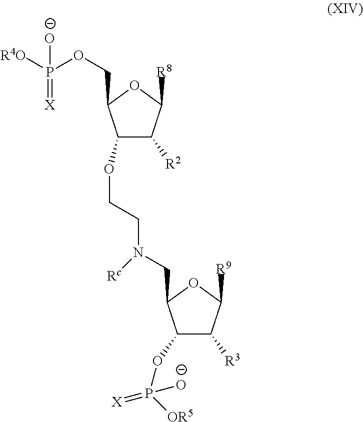

wherein:
X, R², R³, R⁴, R⁵, R⁸, R⁹, and R^{c} are as defined herein.

In some aspects, the present disclosure provides oligonucleotides of the Formula (IX-a):

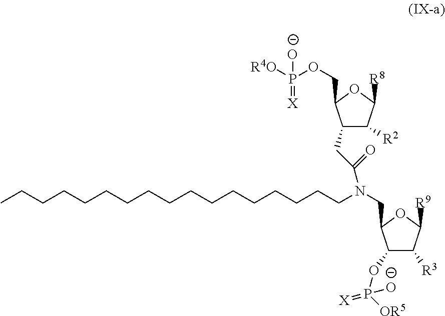

wherein:
X, R², R³, R⁴, R⁵, R⁸, and R⁹ are as defined herein.

In some aspects, the present disclosure provides oligonucleotides of the Formula (IX-b):

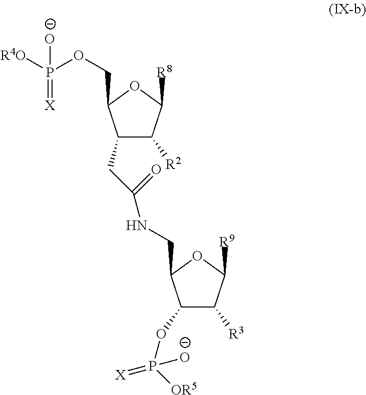

wherein:
X, R², R³, R⁴, R⁵, R⁸, and R⁹ are as defined herein.

In some aspects, the present disclosure provides oligonucleotides of the Formula (X-a):

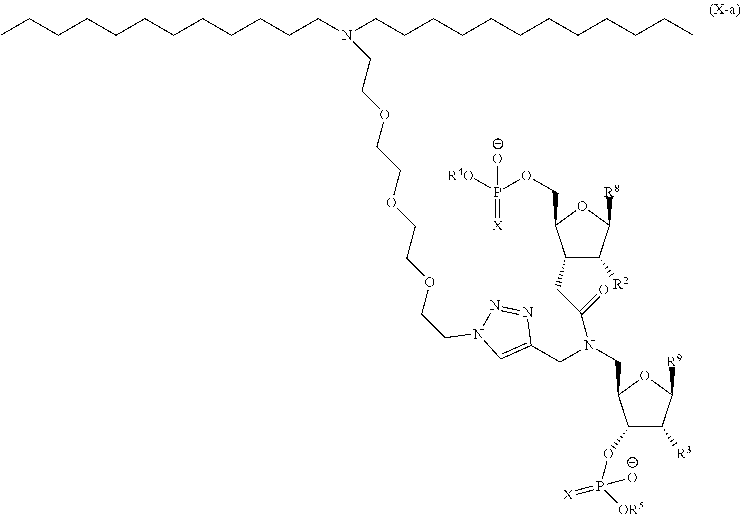

wherein:
X, R², R³, R⁴, R⁵, R⁸, and R⁹ are as defined herein. In some aspects, the present disclosure provides oligonucleotides of the Formula (XV):

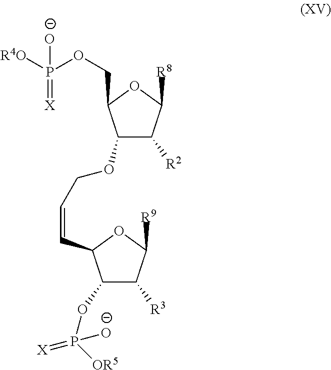

wherein:
X, R², R³, R⁴, R⁵, R⁸, R⁹, and R^{c} are as defined herein.

In some aspects, the present disclosure provides oligonucleotides of the Formula (XVI):

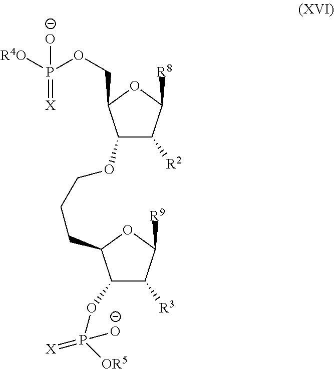

wherein:
X, R², R³, R⁴, R¹, R⁸, and R⁹ are as defined herein. In some aspects, the present disclosure provides oligonucleotides of the Formula (XVII):

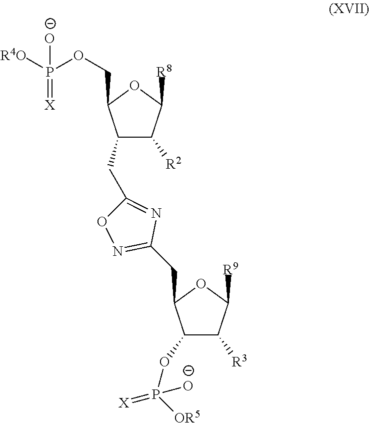

wherein:
X, R², R³, R⁴, R⁵, R⁸, R⁹, and R^{c} are as defined herein.

In some aspects, the present disclosure provides oligonucleotides of the Formula (XVIII):

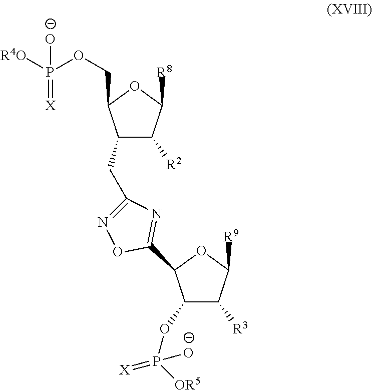

wherein:
X, R², R³, R⁴, R⁵, R⁸, R⁹, and R^{c} are as defined herein.

In some aspects, the present disclosure provides oligonucleotides of the Formula (IX-c):

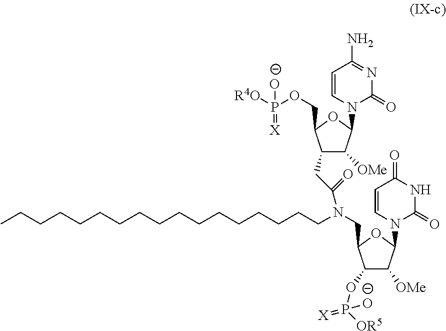

wherein:
X, R², R³, R⁴, R⁵, R⁸, R⁹, and R^{c} are as defined herein.

In some aspects, the present disclosure provides oligonucleotides of the Formula (IX-d):

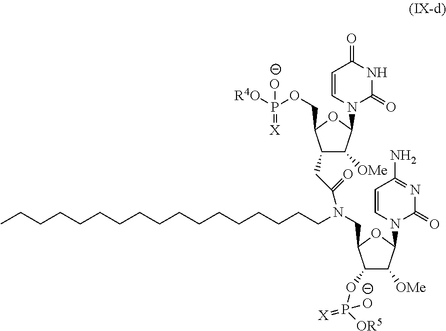

wherein:
X, R², R³, R⁴, R⁵, R⁸, R⁹, and R^{c} are as defined herein. In some aspects, the present disclosure provides oligonucleotides of the Formula (IX-e):

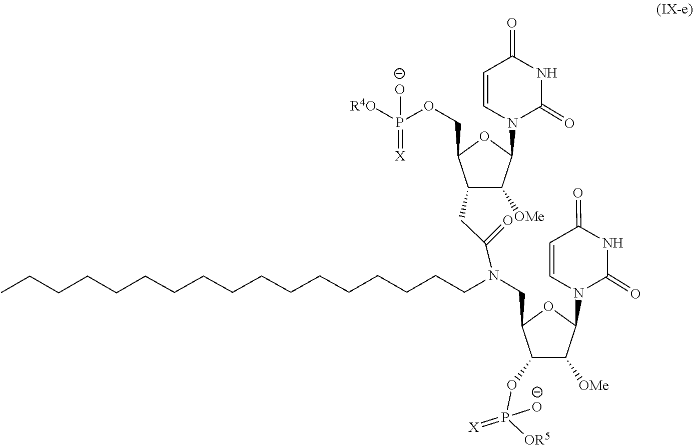

wherein:
X, R², R³, R⁴, R⁵, R⁸, R⁹, and R^{c} are as defined herein.

In some aspects, the present disclosure provides compounds of the formula:

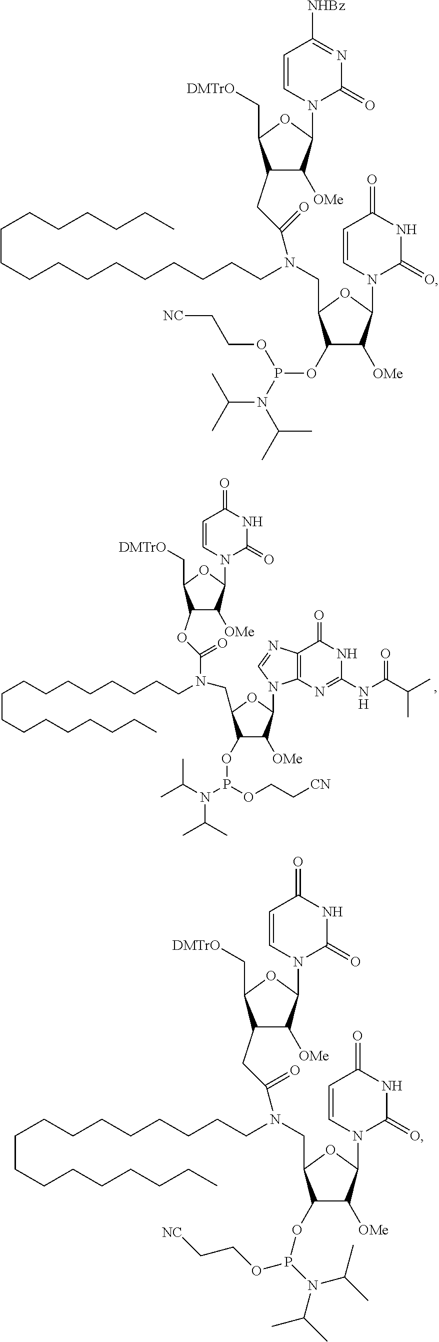

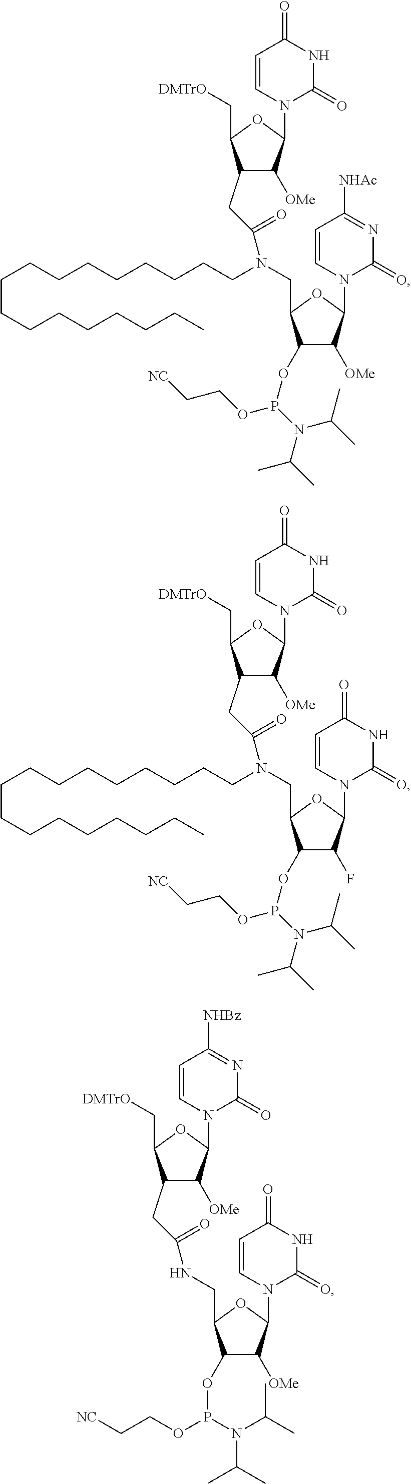

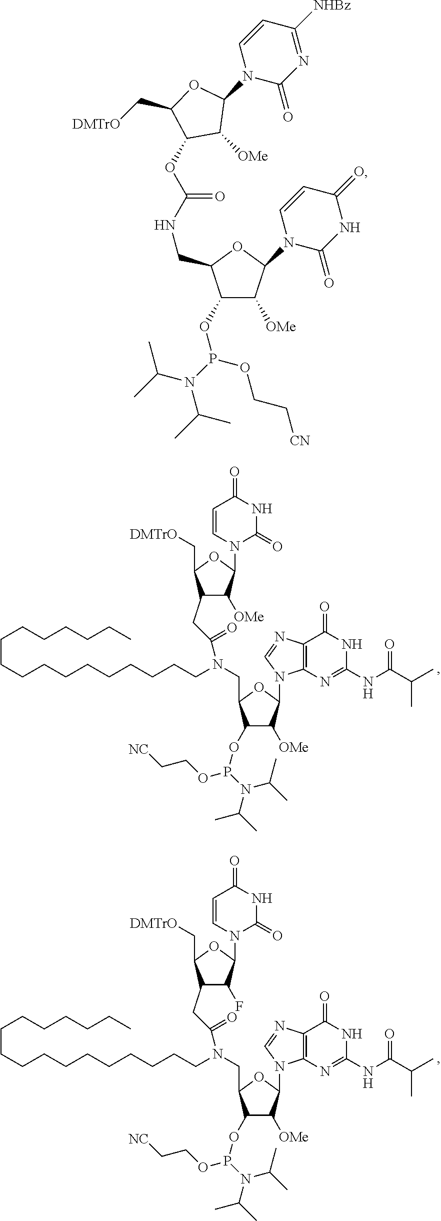

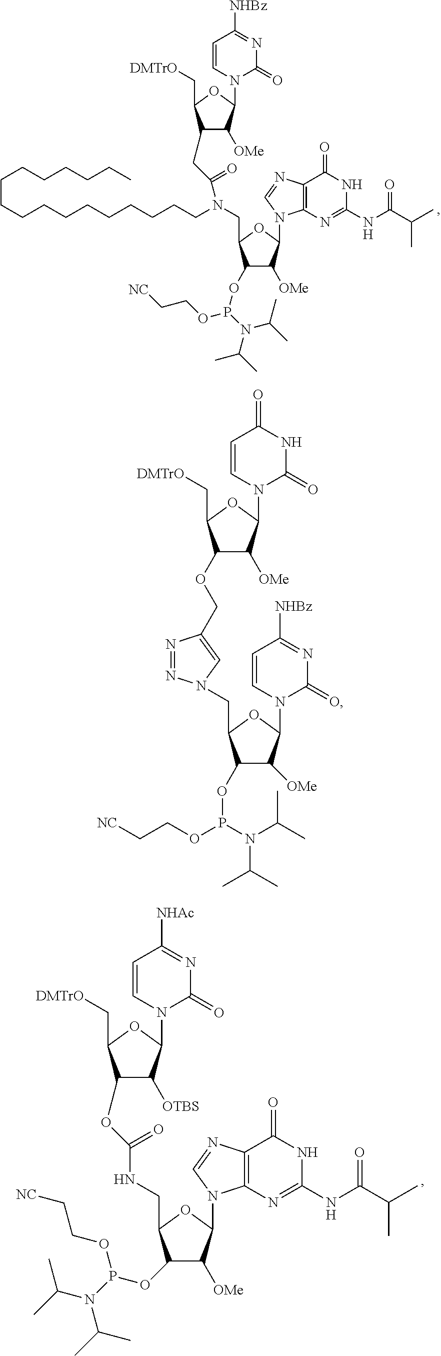

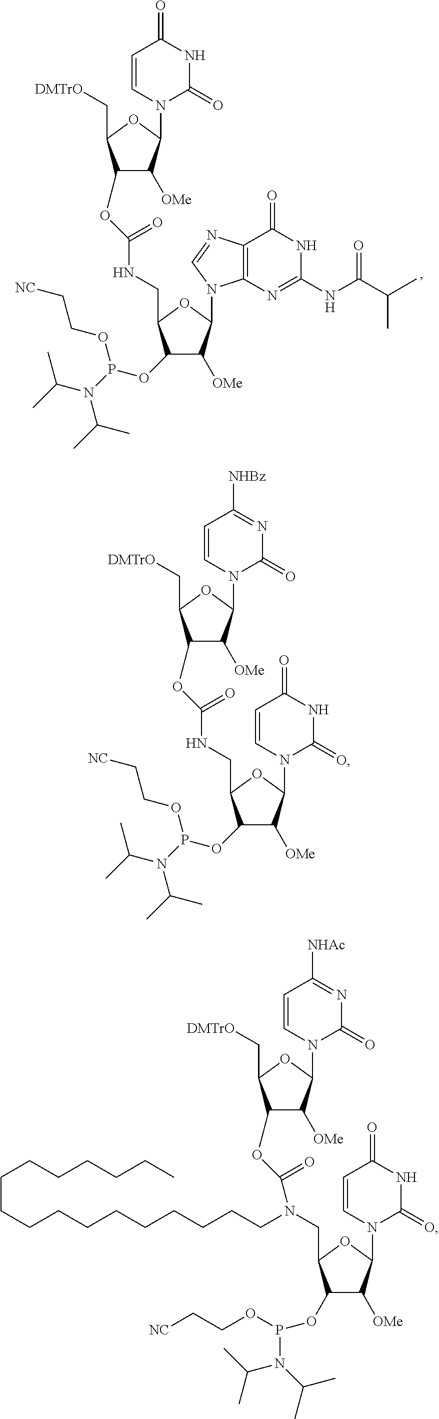

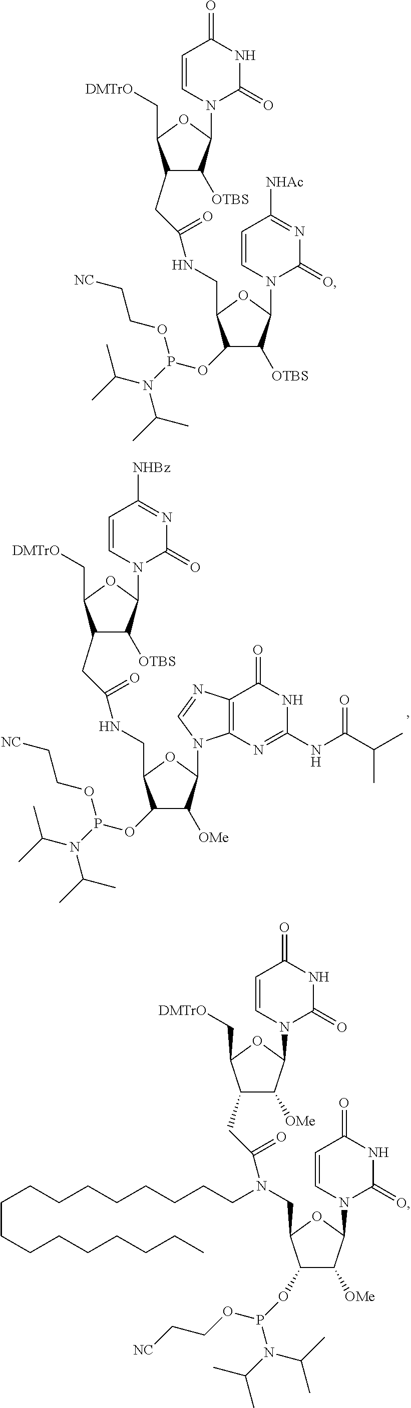

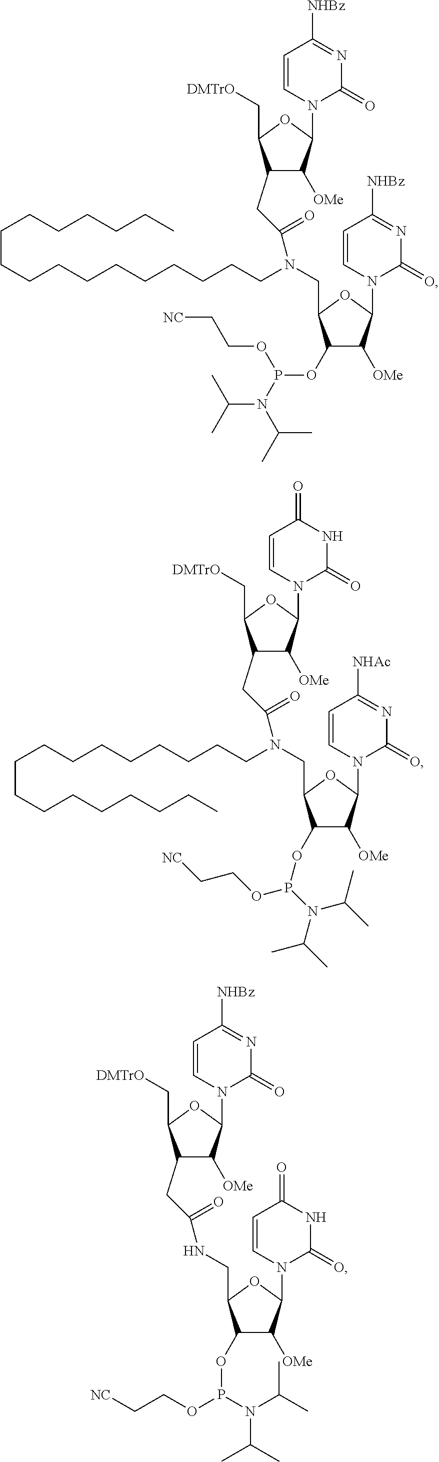

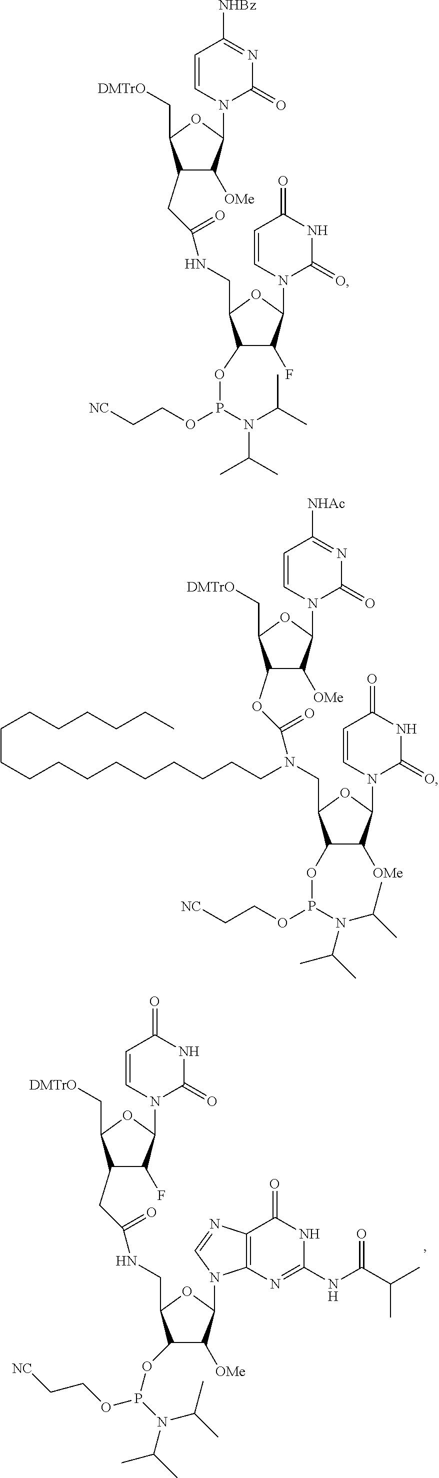

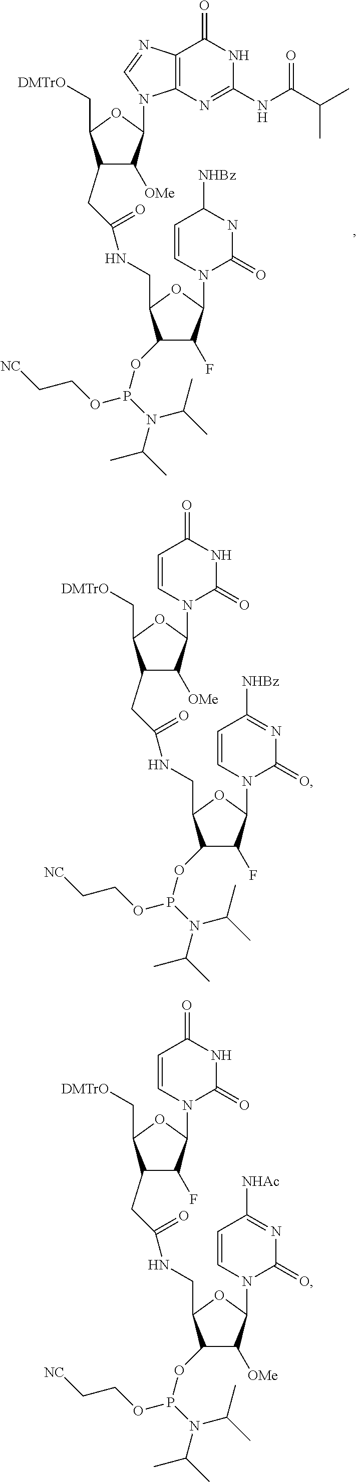

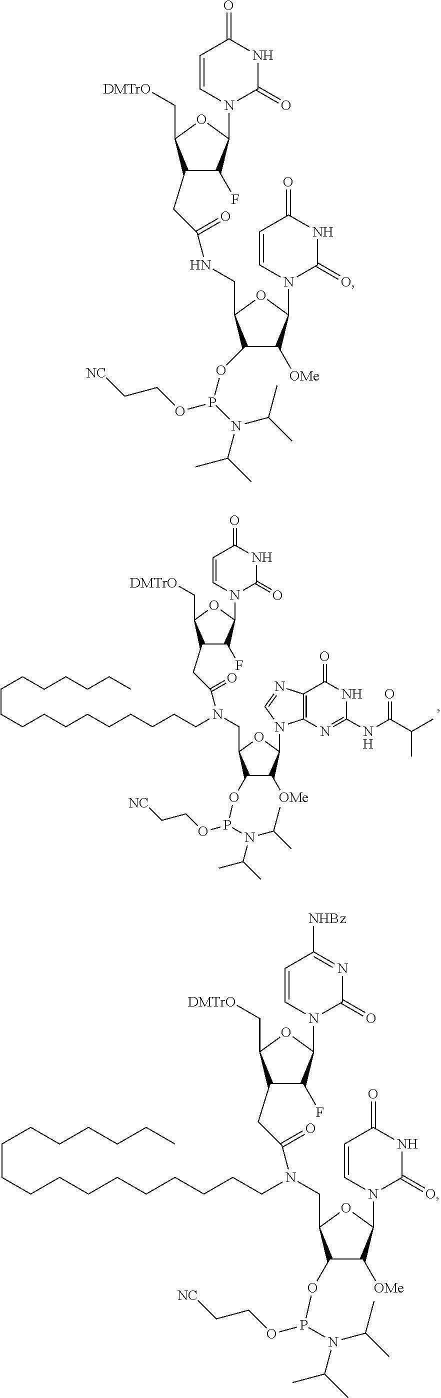

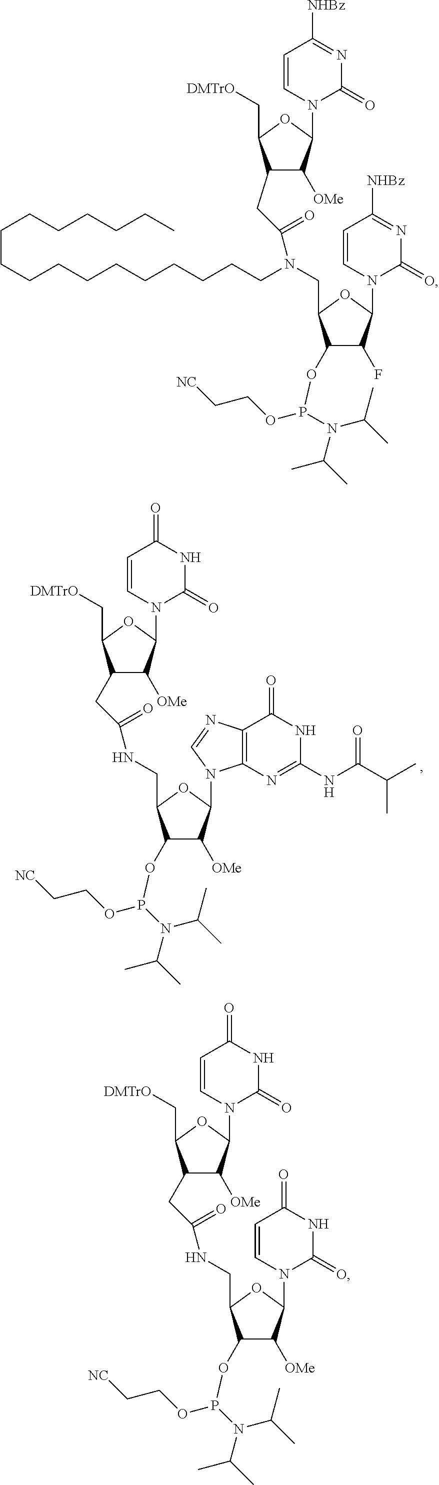

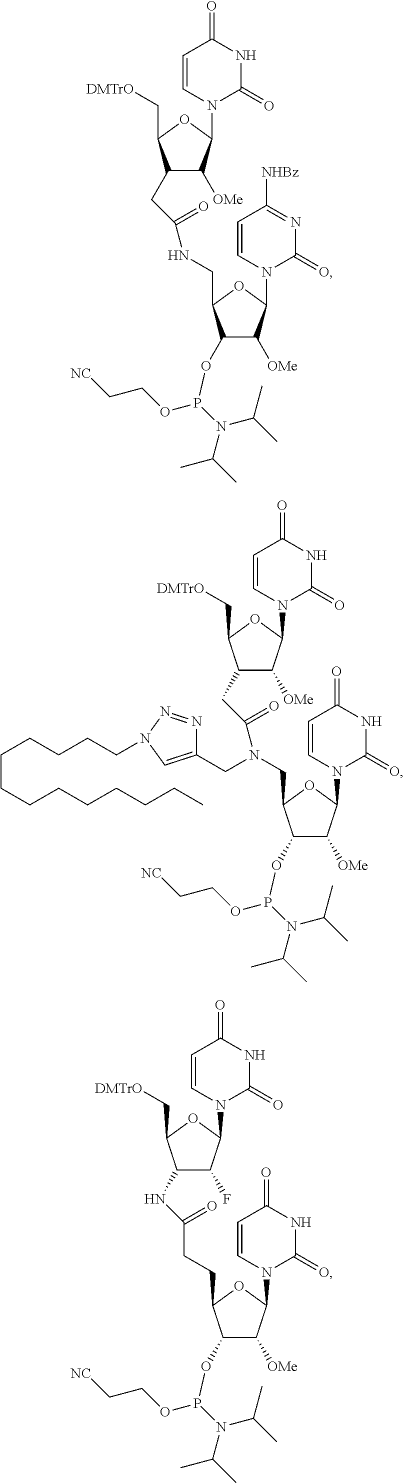

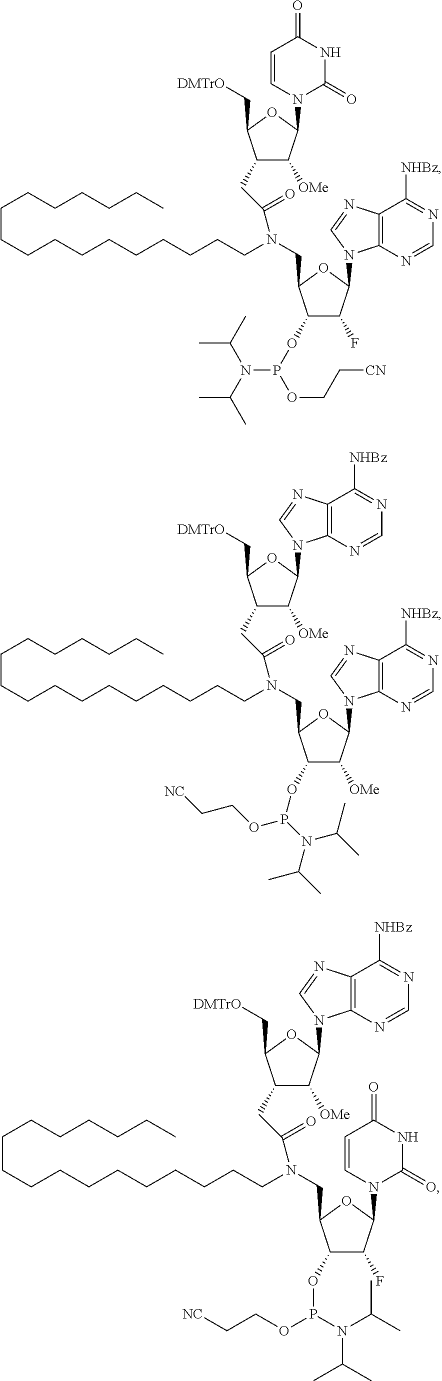

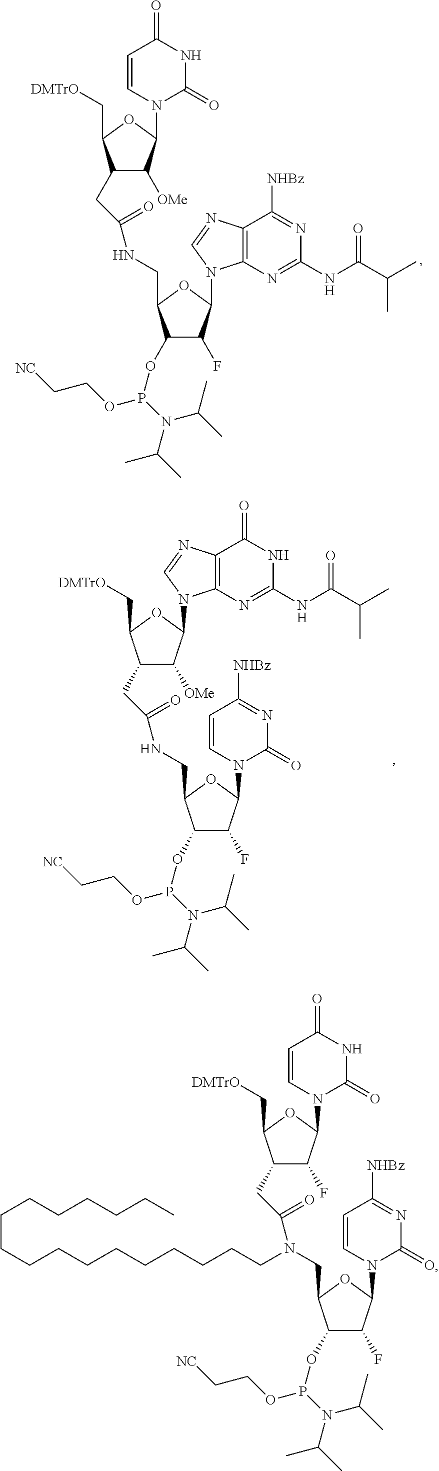

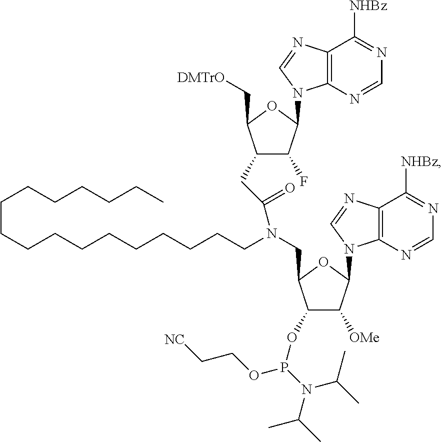

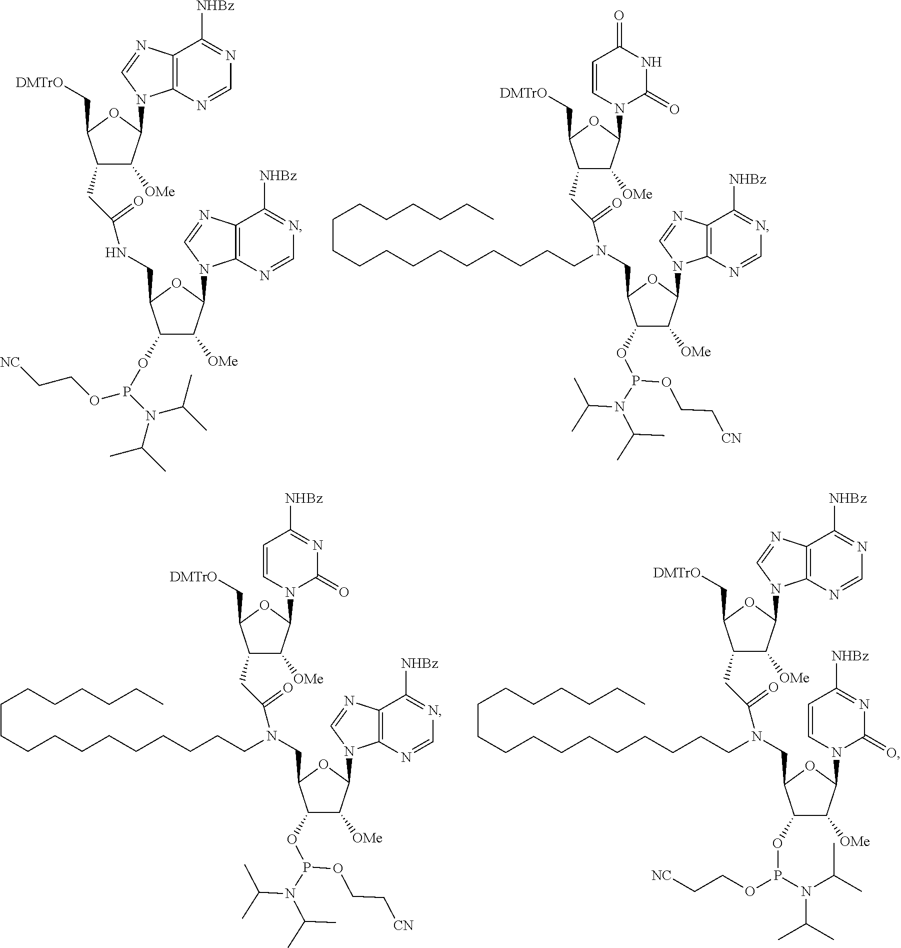

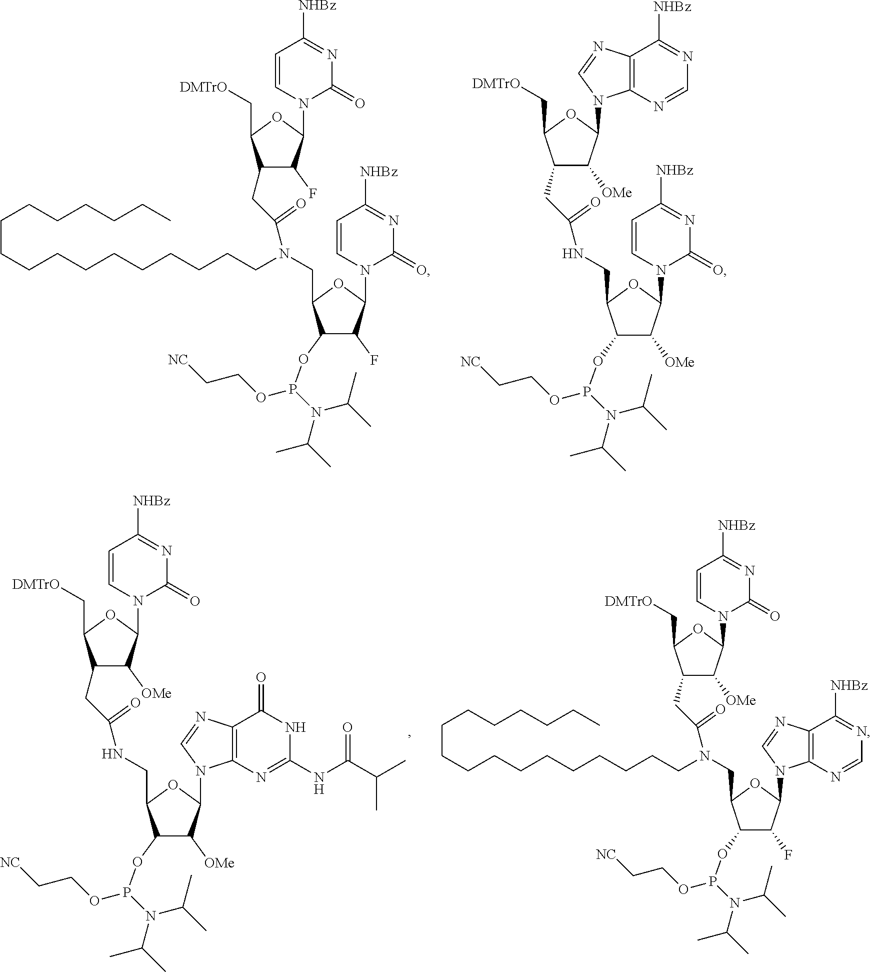

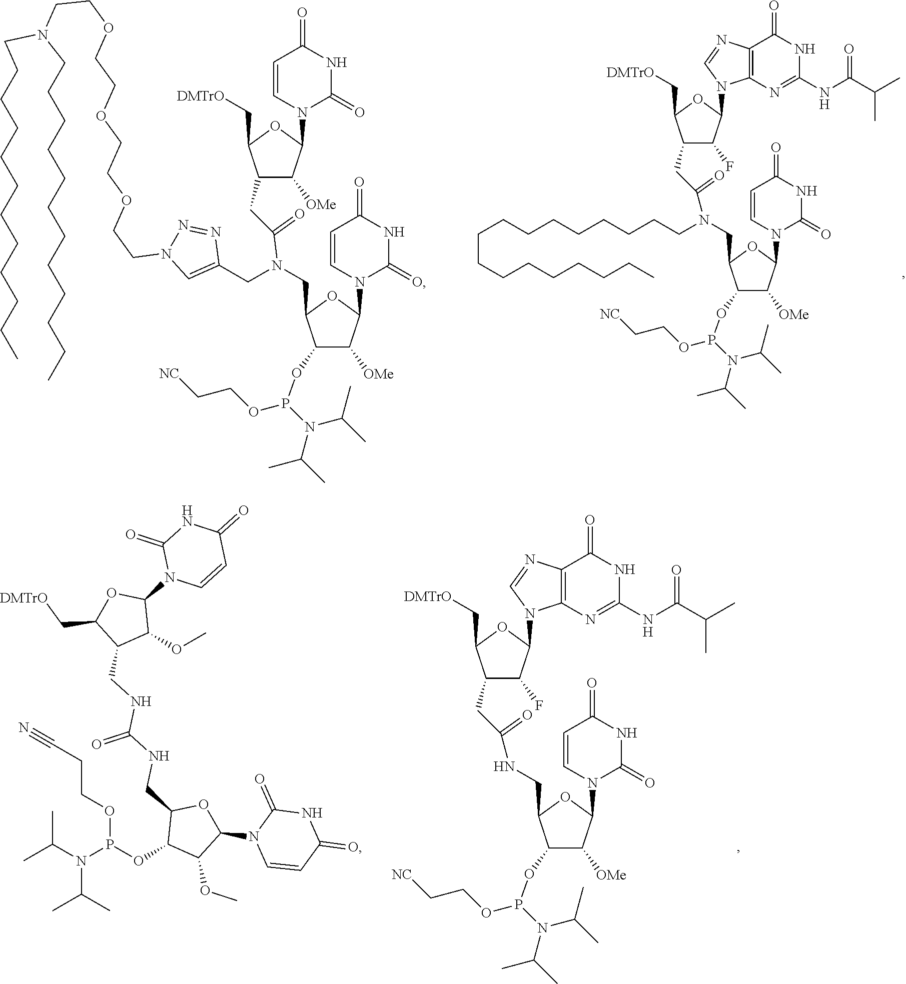

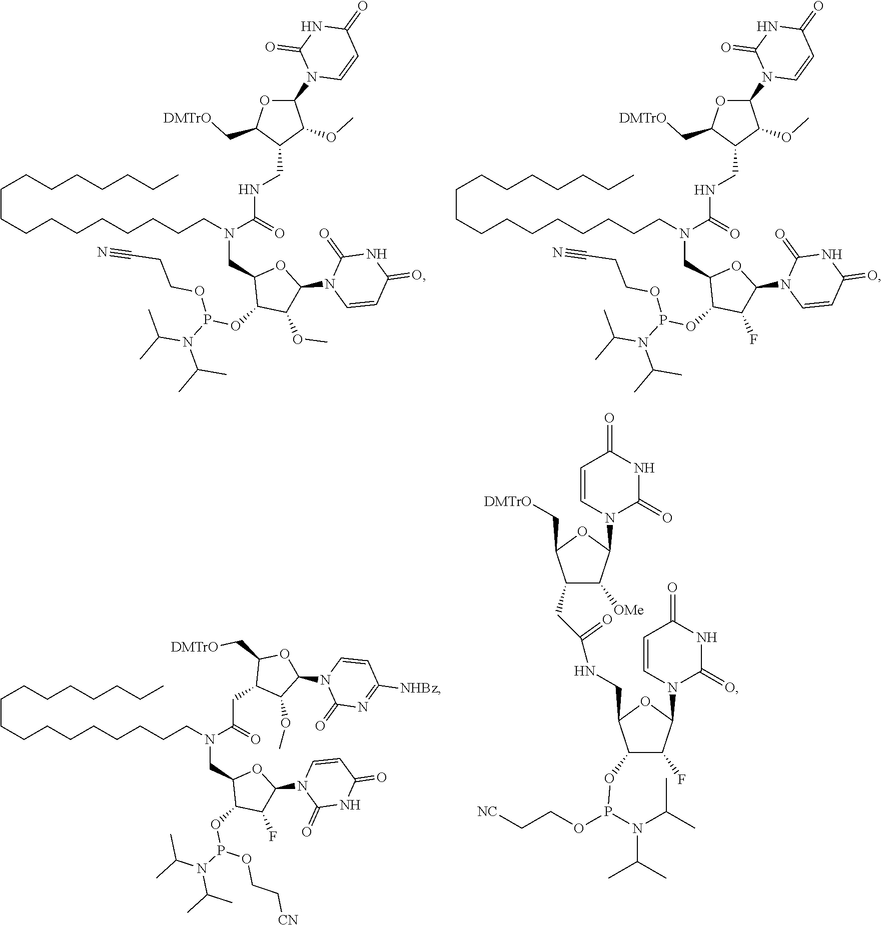

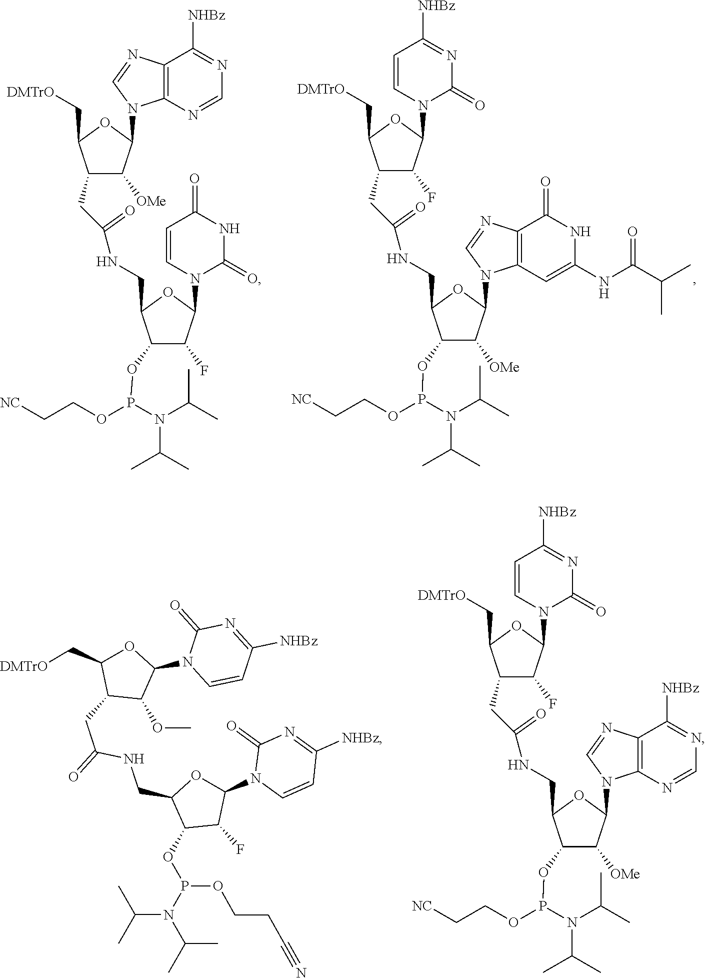

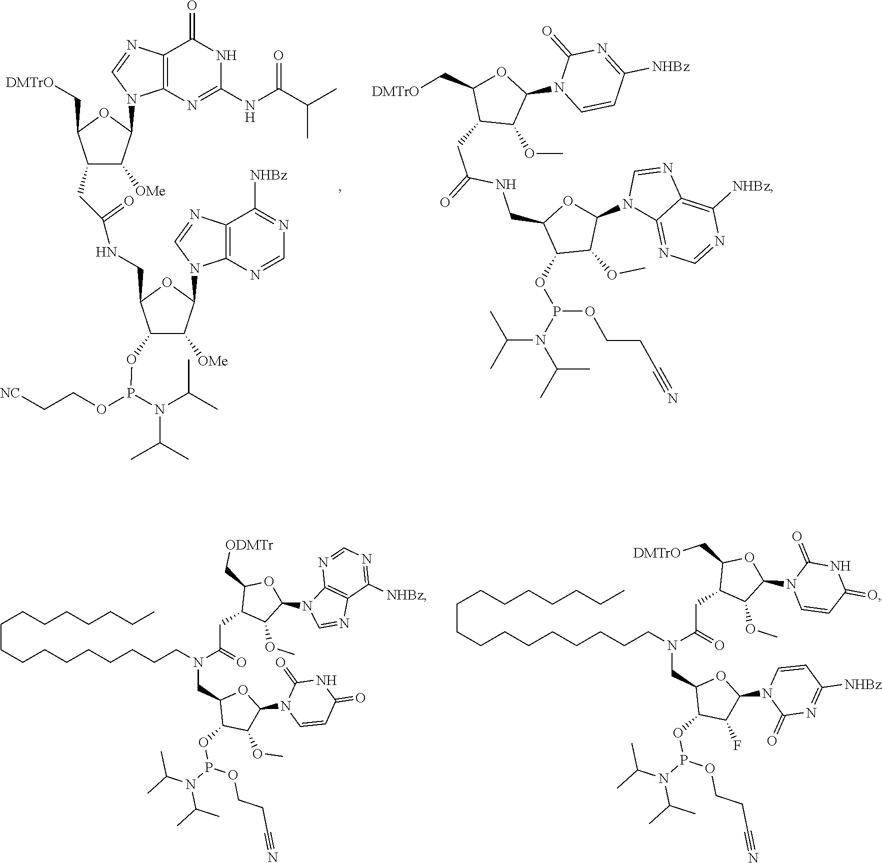

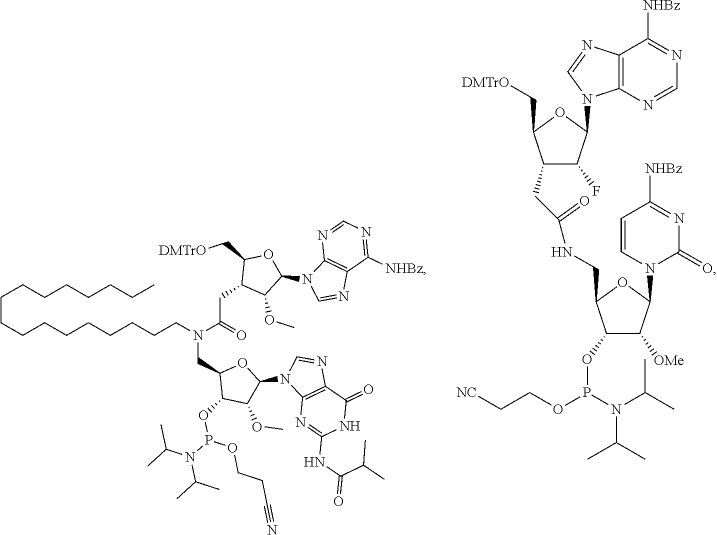

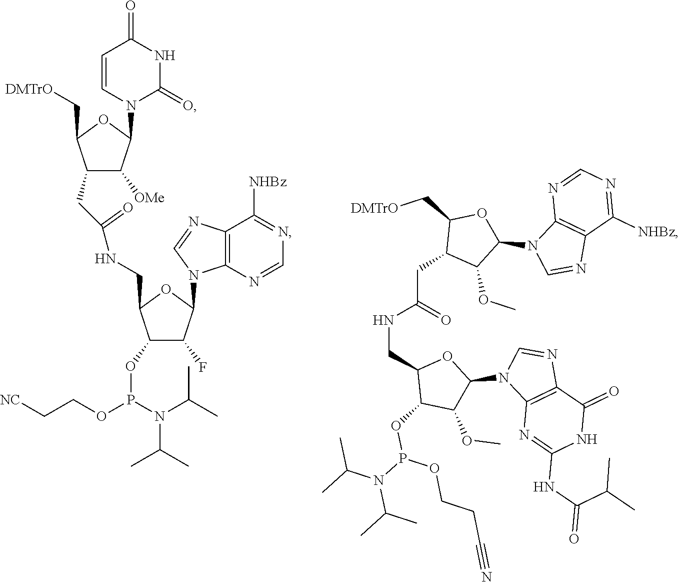

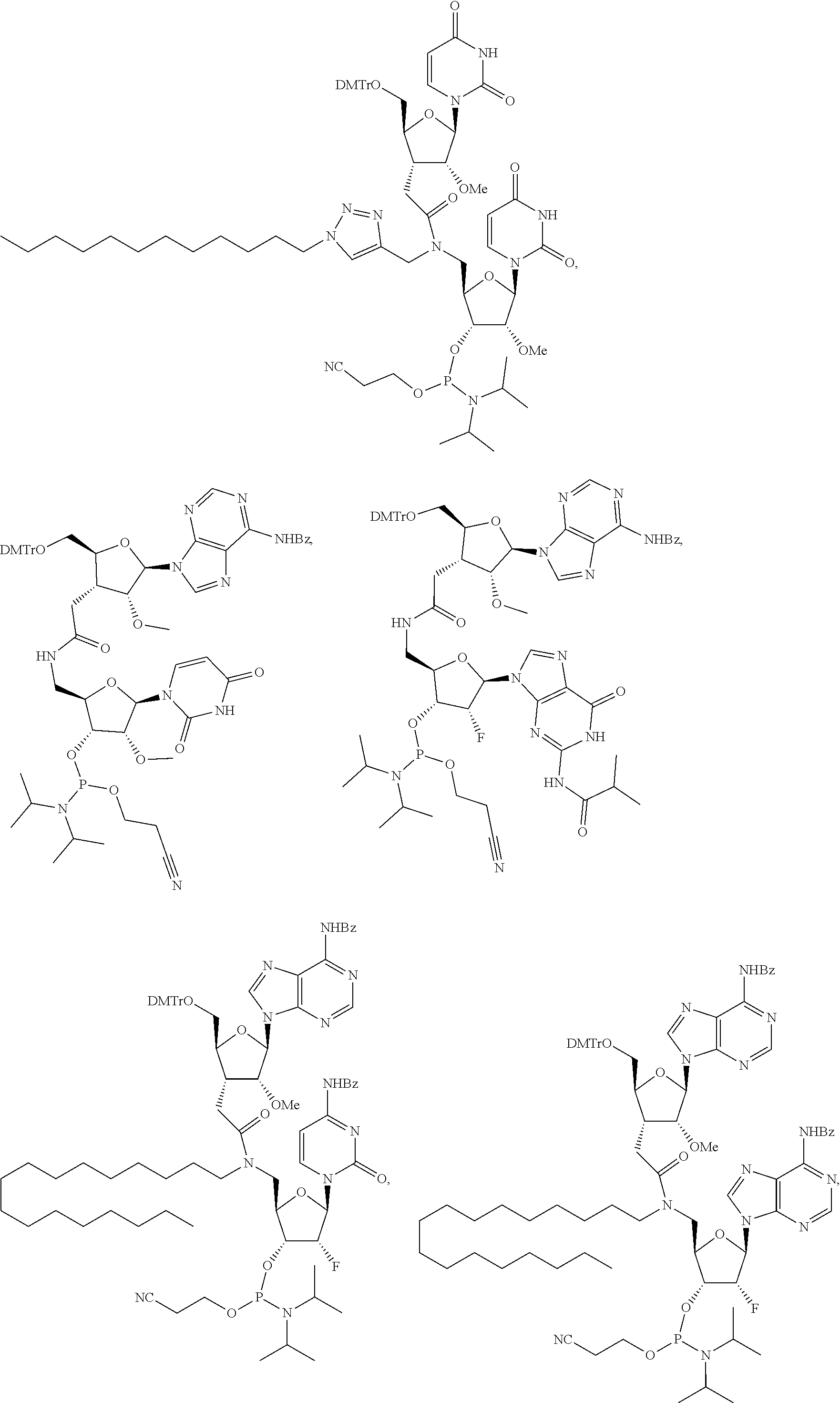

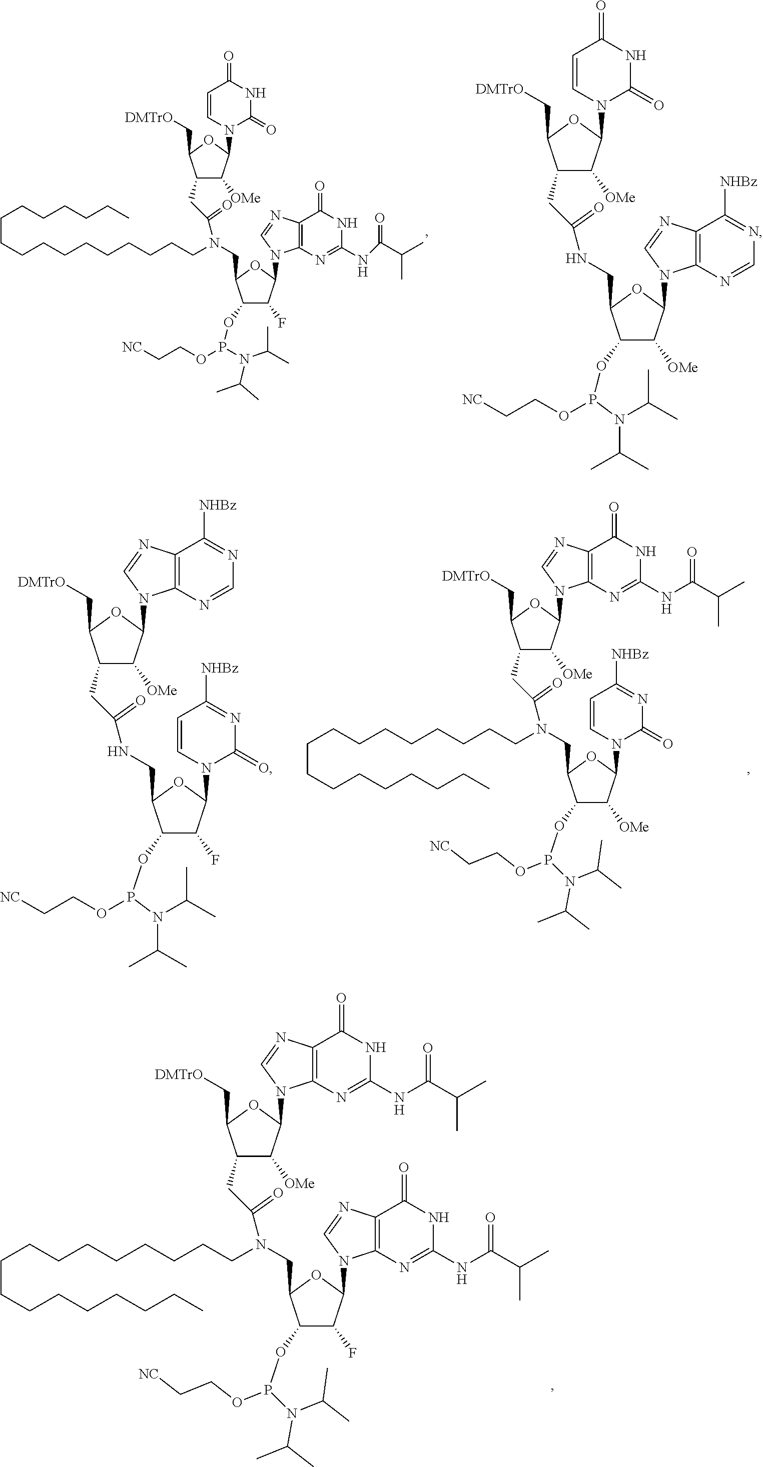

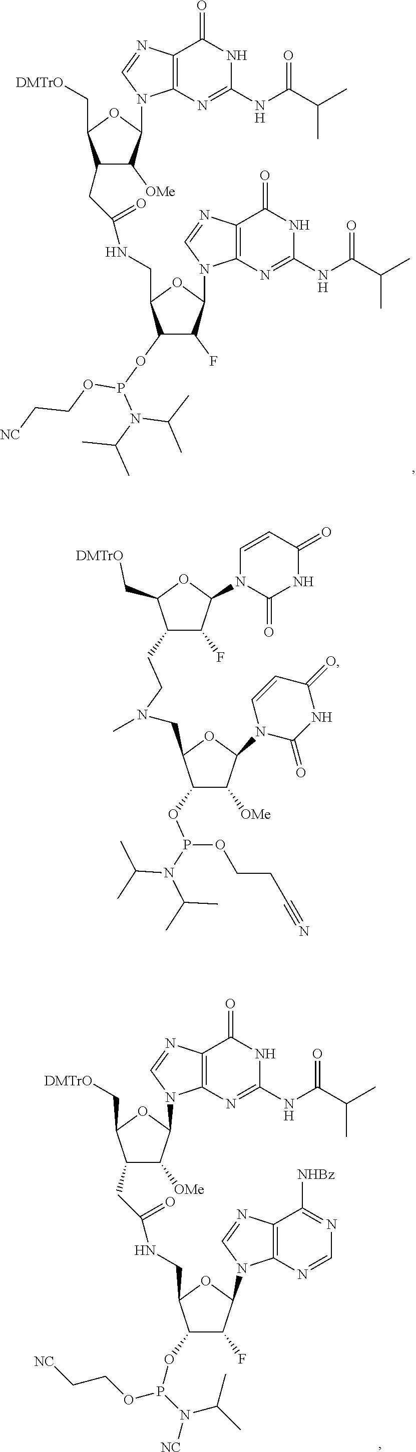

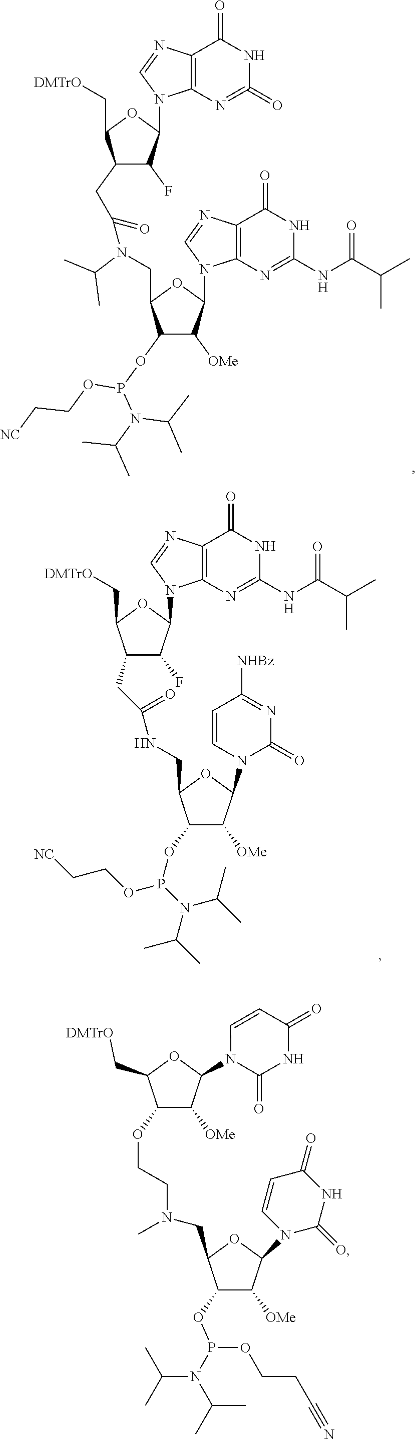

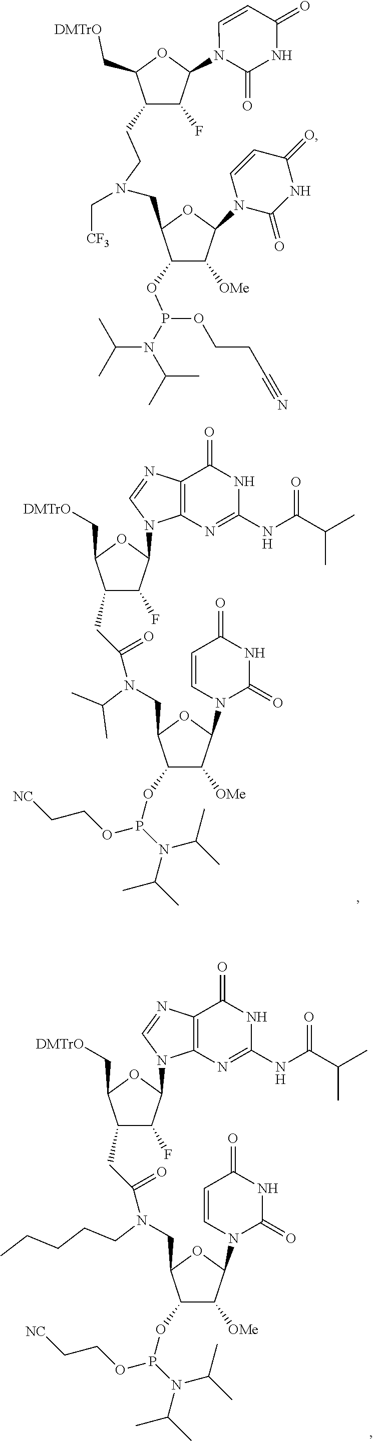

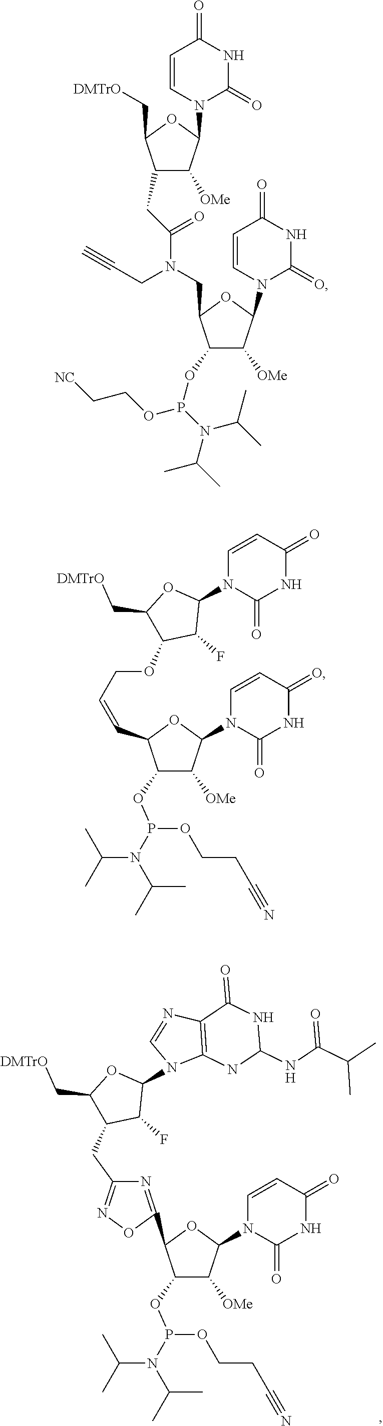

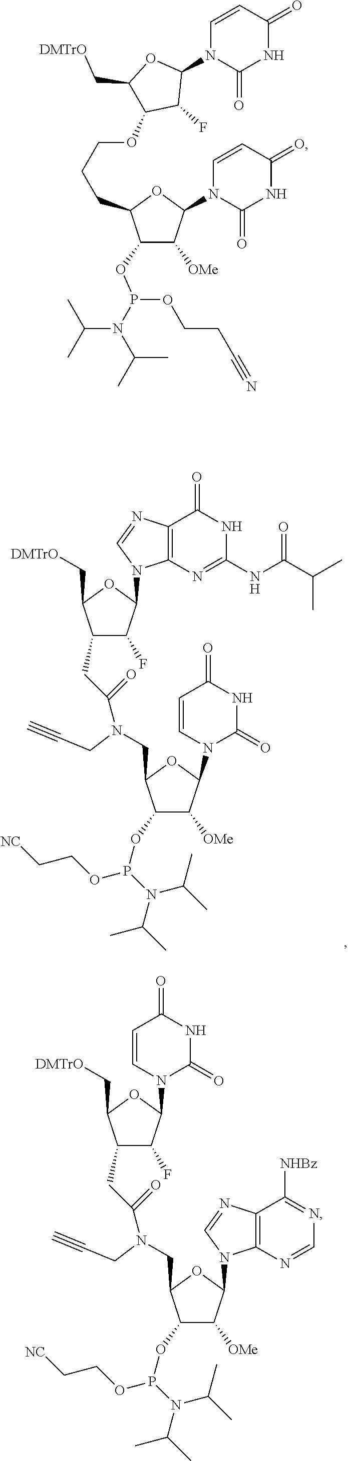

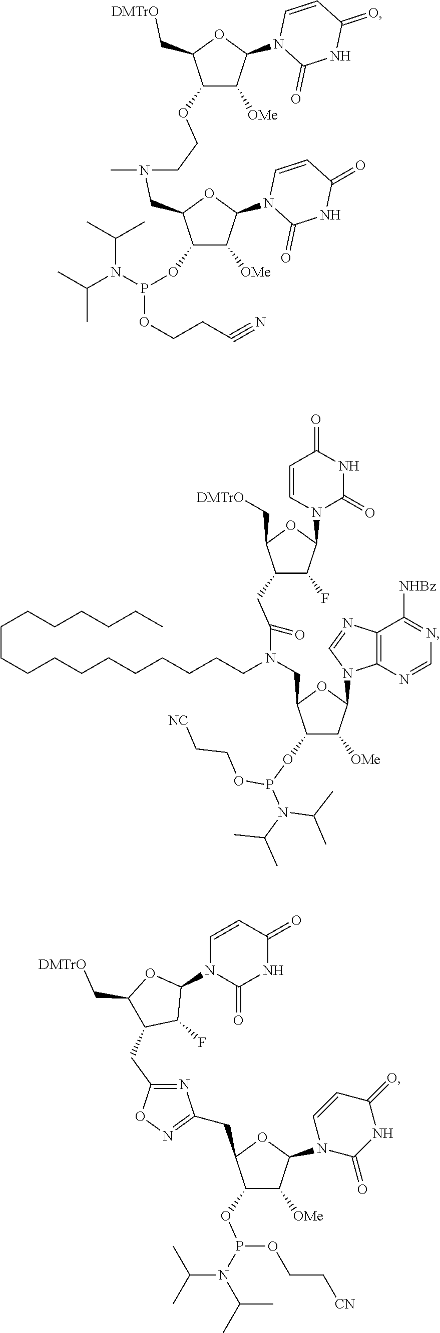

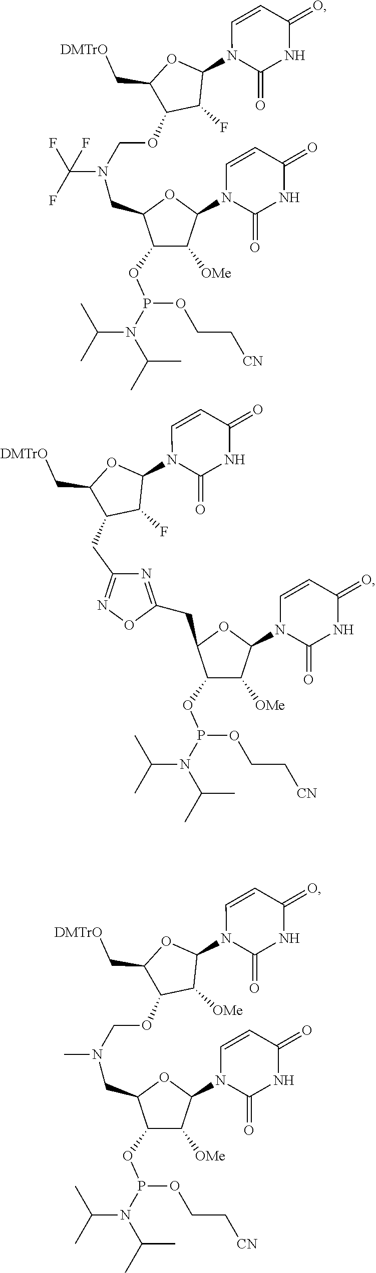

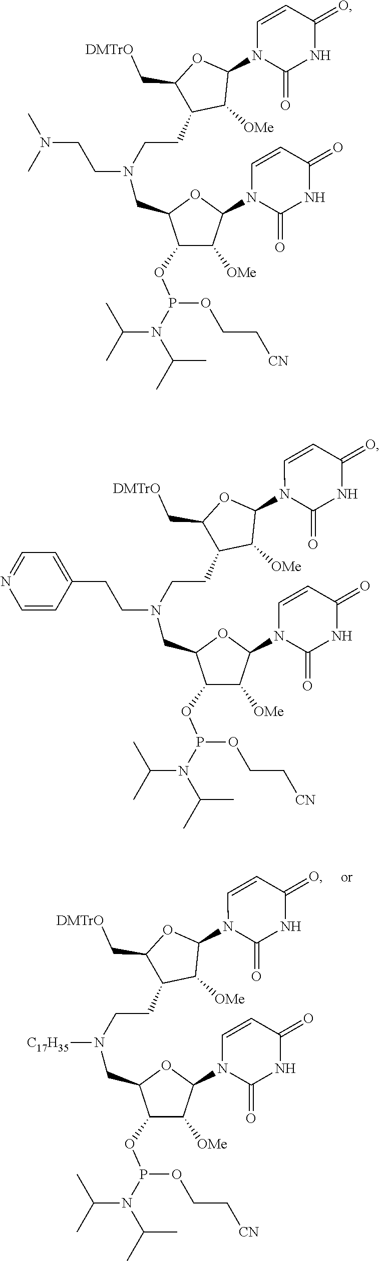

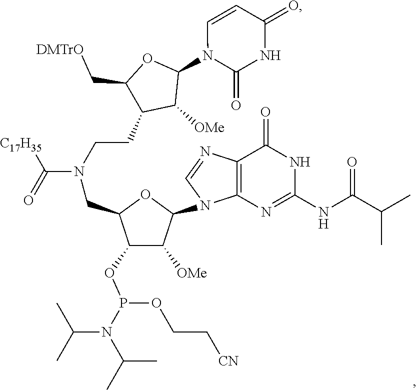

or a salt thereof.

In another aspect, the present disclosure provides compositions comprising any of the compounds provided herein, and a pharmaceutically acceptable excipient.

In some embodiments, R⁴ and R⁵ each comprise an oligonucleotide. In some embodiments, one or both of the oligonucleotides is attached at its 5′ end. In some embodiments, one or both of the oligonucleotides is attached at its 3′ end. In some embodiments, one or both of the oligonucleotides is attached at an internal position on the oligonucleotide. In certain embodiments, the internal position is an internucleoside linkage.

In some embodiments, R⁴ and R⁵ are joined together to form a single oligonucleotide. In some embodiments, R⁴ comprises an oligonucleotide, and R⁵ comprises a protecting group.

In some embodiments, R⁴ comprises a protecting group, and R⁵ comprises an oligonucleotide. In some embodiments, R⁴ and R⁵ each comprise a protecting group.

In certain embodiments, R⁴ is attached at the 3′ end of the oligonucleotide. In certain embodiments, R⁴ is attached at the 5′ end of the oligonucleotide.

In certain embodiments, R⁵ is attached at the 3′ end of the oligonucleotide. In certain embodiments, R⁵ is attached at the 5′ end of the oligonucleotide.

In another aspect, the present disclosure provides methods for delivering a therapeutic oligonucleotide to a subject, comprising administration of any of the compounds or compositions provided herein to the subject. In another aspect, the present disclosure provides methods for delivering a therapeutic oligonucleotide to the brain of a subject, comprising administration of any of the compounds or compositions provided herein to the subject. In another aspect, the present disclosure provides methods for treating or ameliorating a disease, disorder, or symptom thereof in a subject, comprising administration of any of the compounds or compositions provided herein to the subject. In some embodiments, the disease, disorder, or symptom thereof is a central nervous system (CNS) disease, disorder, or symptom thereof. In certain embodiments, the disease, disorder, or symptom thereof is Alzheimer's disease, or a symptom thereof. In some embodiments, the compound is administered to the subject intrathecally.

In another aspect, the present disclosure provides methods for making any of the compounds provided herein, comprising one or more compounds and chemical transformations described herein.

### Definitions

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included,” is not limiting. The section headings used herein are for organizational purposes only and are not to be construed as limiting the subject matter described.

Unless otherwise indicated, the following terms have the following meanings:

As used herein, the term “treating” a disorder encompasses ameliorating, mitigating and/or managing the disorder and/or conditions that may cause the disorder. The terms “treating” and “treatment” refer to a method of alleviating or abating a disease and/or its attendant symptoms. In accordance with the present disclosure, “treating” includes blocking, inhibiting, attenuating, protecting against, modulating, reversing the effects of, and reducing the occurrence of, e.g., the harmful effects of a disorder. As used herein, “inhibiting” encompasses preventing, reducing, and halting progression.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high-performance liquid chromatography (HPLC). Particularly, in certain embodiments, the compound is at least 85% pure, more preferably at least 90% pure, more preferably at least 95% pure, and most preferably at least 99% pure.

The term “administration” or “administering” includes routes of introducing the compound(s) to a subject to perform their intended function. Examples of routes of administration which can be used include injection (subcutaneous, intravenous, parenterally, intraperitoneally, intrathecal), topical, oral, inhalation, rectal, and transdermal.

“Parenteral administration” means administration through injection or infusion. Parenteral administration includes subcutaneous administration, intravenous administration, intramuscular administration, intraarterial administration, intraperitoneal administration, or intracranial administration, e.g., intrathecal or intracerebroventricular administration.

“Pharmaceutically acceptable carrier or diluent” means any substance suitable for use in administering to an individual. In certain embodiments, a pharmaceutically acceptable carrier or diluent aids the administration of a compound to and absorption by an individual and can be included in the compositions of the present disclosure without causing a significant adverse toxicological effect on the patient. Non-limiting examples of pharmaceutically acceptable excipients include water, NaCl, normal saline solutions, and the like. For example, a pharmaceutically acceptable carrier can be a sterile aqueous solution, such as PBS or water-for-injection. One of skill in the art will recognize that other pharmaceutical excipients are useful in the present disclosure.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response. An effective amount is also one in which any non-tolerable or detrimental effects (e.g., side effects) of the compound are outweighed by the therapeutically beneficial effects.

The phrases “systemic administration,” “administered systemically,” “peripheral administration” and “administered peripherally” as used herein mean the administration of a compound(s), oligonucleotide(s), drug, or other material, such that it enters the patient's circulatory system and, thus, is subject to metabolism and other like processes.

The term “therapeutically effective amount” refers to the amount of the compound being administered sufficient to prevent development of or alleviate to some extent one or more of the symptoms of the condition or disorder being treated.

A therapeutically effective amount of compound (i.e., an effective dosage) may range from about 0.005 g/kg to about 200 mg/kg, preferably about 0.01 mg/kg to about 200 mg/kg, and more preferably about 0.015 mg/kg to about 30 mg/kg of body weight. In other embodiments, the therapeutically effect amount may range from about 1.0 pM to about 10 μM.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound can include a single treatment or, preferably, can include a series of treatments. In one example, a subject is treated with a compound in the range of between about 0.005 μg/kg to about 200 mg/kg of body weight, daily, weekly, monthly, quarterly, or yearly. In another example, a subject may be treated daily, weekly, monthly, quarterly, or yearly for several years in the setting of a chronic condition or illness. It will also be appreciated that the effective dosage of a compound used for treatment may increase or decrease over the course of a particular treatment.

The term “chiral” refers to molecules that have the property of non-superimposability of the mirror image partner, while the term “achiral” refers to molecules that are superimposable on their mirror image partner.

The term “diastereomers” refers to stereoisomers with two or more centers of dissymmetry and whose molecules are not mirror images of one another.

The term “enantiomers” refers to two stereoisomers of a compound that are non-superimposable mirror images of one another. An equimolar mixture of two enantiomers is called a “racemic mixture” or a “racemate.”

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents or resolved using conventional techniques. When the compounds described herein contain olefinic bonds or other centers of geometric asymmetry, and unless specified otherwise, it is intended that the compounds include both E and Z geometric isomers.

The term “tautomer,” as used herein, refers to one of two or more structural isomers which exist in equilibrium, and which are readily converted from one isomeric form to another.

It will be apparent to one skilled in the art that certain compounds of this disclosure may exist in tautomeric forms, all such tautomeric forms of the compounds being within the scope of the disclosure.

The terms “isomers” or “stereoisomers” refer to compounds that have identical chemical constitution but differ with regard to the arrangement of the atoms or groups in space.

The term “prodrug” is meant to indicate a compound that may be converted under physiological conditions or by solvolysis to a biologically active nucleic acid or analogue thereof described herein. Thus, the term “prodrug” refers to a precursor of a biologically active nucleic acid or analogue thereof that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., DESIGN OF PRODRUGS (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in BIOREVERSIBLE CARRIERS IN DRUG DESIGN, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of suitable prodrugs include, but are not limited to glutathione, acyloxy, thioacyloxy, 2-carboalkoxyethyl, disulfide, thiaminal, and enol ester derivatives of a phosphorus atom-modified nucleic acid. The term “pro-oligonucleotide” or “pronucleotide” or “nucleic acid prodrug” refers to an oligonucleotide which has been modified to be a prodrug of the oligonucleotide. Phosphonate and phosphate prodrugs can be found, for example, in Wiener et al., “Prodrugs or phosphonates and phosphates: crossing the membrane” TOP. CURR. CHEM. 2015, 360:115-160, the entirety of which is herein incorporated by reference.

In certain aspects, the compounds of the present disclosure are prodrugs of any of the formulae herein.

The term “subject” refers to animals such as mammals, including, but not limited to, primates (e.g., humans), cows, sheep, goats, horses, dogs, cats, rabbits, rats, mice, and the like. In certain embodiments, the subject is a human.

The terms “a,” “an,” and “the” refer to “one or more” when used in this application, including the claims. Thus, for example, reference to “a sample” includes a plurality of samples, unless the context clearly is to the contrary (e.g., a plurality of samples), and so forth.

Throughout this specification and the claims, the words “comprise,” “comprises,” and “comprising” are used in a non-exclusive sense, except where the context requires otherwise.

As used herein, the term “about,” when referring to a value, is meant to encompass variations of, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “aliphatic” includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, the term “alkyl” includes straight, branched and cyclic alkyl groups. An analogous convention applies to other generic terms such as “alkenyl”, “alkynyl”, and the like. Furthermore, the terms “alkyl”, “alkenyl”, “alkynyl”, and the like encompass both substituted and unsubstituted groups. In certain embodiments, “lower alkyl” is used to indicate those alkyl groups (cyclic, acyclic, substituted, unsubstituted, branched or unbranched) having 1-6 carbon atoms.

In certain embodiments, the alkyl, alkenyl, and alkynyl groups employed in the disclosure contain 1-30 aliphatic carbon atoms. In certain other embodiments, the alkyl, alkenyl, and alkynyl groups employed in the disclosure contain 1-20 aliphatic carbon atoms. In yet other embodiments, the alkyl, alkenyl, and alkynyl groups employed in the disclosure contain 10-30 aliphatic carbon atoms. In still other embodiments, the alkyl, alkenyl, and alkynyl groups employed in the disclosure contain 10-20 aliphatic carbon atoms. In yet other embodiments, the alkyl, alkenyl, and alkynyl groups employed in the disclosure contain 1-10 carbon atoms. Illustrative aliphatic groups thus include, but are not limited to, for example, methyl, ethyl, n-propyl, isopropyl, cyclopropyl, —CH₂-cyclopropyl, vinyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

As used herein, the term “alkyl,” by itself or as part of another substituent, means, unless otherwise stated, a straight-chained (i.e., unbranched) or branched carbon chain (or carbon), or combination thereof, which may be fully saturated, mono-, (e.g., alkene or alkenyl) or polyunsaturated (e.g., alkyne or alkynyl) and can include mono-, di- and multivalent radicals, having the number of carbon atoms designated. For example, C₁-C₃₀ means 1 to 30 carbon atoms. A specified number of carbon atoms within this range includes, for example, C₁-C₃₀ alkyl (having 1-20 carbon atoms), C₁-C₂₀ alkyl (having 1-20 carbon atoms), C₁-C₁₂ alkyl (having 1-12 carbon atoms) and C₁-C₄ alkyl (having 1-4 carbon atoms), and Cis (having 18 carbon atoms).

The term “alkenyl” refers to an unsaturated hydrocarbon chain that may be a straight chain or branched chain, containing at least 2 carbon atoms and at least one carbon-carbon double bond (e.g., containing 2 to 30 carbon atoms and at least one carbon-carbon double bond). Alkenyl groups may be substituted or unsubstituted with one or more substituents.

The term “alkynyl” refers to an unsaturated hydrocarbon chain that may be a straight chain or branched chain, containing at least 2 carbon atoms and at least one carbon-carbon triple bond (e.g., containing 2 to 30 carbon atoms and at least one carbon-carbon triple bond). Alkynyl groups may be substituted with one or more substituents or unsubstituted.

The term “lower alkyl” refers to a C₁-C₆ alkyl chain. Examples of alkyl groups include methyl, ethyl, n-propyl, isopropyl, tert-butyl, and n-pentyl. Alkyl groups may be substituted with one or more substituents or unsubstituted.

The term “haloalkyl” refers to an alkyl group that is substituted by one or more halo substituents. Examples of haloalkyl groups include fluoromethyl, difluoromethyl, trifluoromethyl, bromomethyl, chloromethyl, and 2,2,2-trifluoroethyl.

The term “arylalkenyl” refers to an unsaturated hydrocarbon chain that may be a straight chain or branched chain, containing 2 to 12 carbon atoms and at least one carbon-carbon double bond wherein one or more of the sp² hybridized carbons of the alkenyl unit attaches to an aryl moiety. Alkenyl groups may be substituted or unsubstituted with one or more substituents.

The term “arylalkynyl” refers to an unsaturated hydrocarbon chain that may be a straight chain or branched chain, containing 2 to 12 carbon atoms and at least one carbon-carbon triple bond wherein one or more of the sp-hybridized carbons of the alkynyl unit attaches to an aryl moiety. Alkynyl groups may be substituted with one or more substituents or unsubstituted.

The sp²- or sp-hybridized carbons of an alkenyl group or an alkynyl group, respectively, may optionally be the point of attachment of the alkenyl or alkynyl groups.

The term “alkoxy” refers to an —O-alkyl substituent.

As used herein, the terms “halogen,” “hal,” or “halo” mean —F, —Cl, —Br or —I.

The term “alkylthio” refers to an —S-alkyl substituent.

The term “alkoxyalkyl” refers to an -alkyl-O-alkyl substituent.

The term “haloalkoxy” refers to an —O-alkyl that is substituted by one or more halo substituents. Examples of haloalkoxy groups include trifluoromethoxy, and 2,2,2-trifluoroethoxy.

The term “haloalkoxyalkyl” refers to an -alkyl-O-alkyl′ where the alkyl′ is substituted by one or more halo substituents.

The term “haloalkylaminocarbonyl” refers to a —C(O)-amino-alkyl where the alkyl is substituted by one or more halo substituents.

The term “haloalkylthio” refers to an —S-alkyl that is substituted by one or more halo substituents. Examples of haloalkylthio groups include trifluoromethylthio, and 2,2,2-trifluoroethylthio.

The term “haloalkylcarbonyl” refers to an —C(O)-alkyl that is substituted by one or more halo substituents. An example of a haloalkylcarbonyl group includes trifluoroacetyl.

The term “cycloalkyl” refers to a hydrocarbon 3-8 membered monocyclic or 7-14 membered bicyclic ring system having at least one saturated ring or having at least one non-aromatic ring, wherein the non-aromatic ring may have some degree of unsaturation.

Cycloalkyl groups may be substituted or unsubstituted with one or more substituents. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclopentenyl, cyclopentadienyl, cyclohexenyl, cyclohexadienyl, and the like.

The term “cycloalkoxy” refers to an —O-cycloalkyl substituent.

The term “cycloalkoxyalkyl” refers to an -alkyl-O-cycloalkyl substituent.

The term “cycloalkylalkoxy” refers to an —O-alkyl-cycloalkyl substituent.

The term “cycloalkylaminocarbonyl” refers to an —C(O)—NH-cycloalkyl substituent.

The term “aryl” refers to a hydrocarbon monocyclic, bicyclic, or tricyclic aromatic ring system. Aryl groups may be substituted or unsubstituted with one or more substituents.

In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

The term “aryloxy” refers to an —O-aryl substituent.

The term “arylalkoxy” refers to an —O-alkyl-aryl substituent.

The term “arylalkylaminocarbonyl” refers to a —C(O)-amino-alkyl-aryl substituent.

The term “aryloxyalkyl” refers to an -alkyl-O-aryl substituent.

The term “alkylaryl” refers to an -aryl-alkyl substituent.

The term “arylalkyl” refers to an -alkyl-aryl substituent.

The term “heteroalkyl” by itself or in combination with another term, means, unless otherwise stated, a stable straight or branched chain, or combinations thereof, including at least one carbon atom and at least one heteroatom (e.g., O, N, P, Si, and/or S), and wherein the nitrogen and sulfur atoms may optionally be oxidized, and the nitrogen heteroatom may optionally be quaternized. The heteroatom(s) (e.g., O, N, P, Si, and/or S) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, CH₂—CH₂, —S(O)—CH₃, —CH₂—CH₂—S(O)₂—CH₃, CH═CH—O—CH₃, Si(CH₃)₃, CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. “Cycloalkyl” is also meant to refer to bicyclic and polycyclic hydrocarbon rings such as, for example, bicyclo[2.2.1]heptane, bicyclo[2.2.2]octane, etc

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, said heteroatoms selected from O, N, or S, and the remainder ring atoms being carbon (with appropriate hydrogen atoms unless otherwise indicated). Heteroaryl groups may be substituted or unsubstituted with one or more substituents. In one embodiment, 0, 1, 2, 3, or 4 atoms of each ring of a heteroaryl group may be substituted by a substituent. Heteroaryl groups may be fully unsaturated, or they may be partially unsaturated and partially saturated. Examples of heteroaryl groups include pyridyl, furanyl, thienyl, pyrrolyl, oxazolyl, oxadiazolyl, imidazolyl thiazolyl, isoxazolyl, quinolinyl, pyrazolyl, isothiazolyl, pyridazinyl, pyrimidinyl, pyrazinyl, triazinyl, isoquinolinyl, indazolyl, and the like.

The term “heteroarylalkyl” refers to an -alkyl-heteroaryl substituent.

The term “heteroaryloxy” refers to an —O-heteroaryl substituent.

The term “heteroarylalkoxy” refers to an —O-alkyl-heteroaryl substituent.

The term “heteroaryloxyalkyl” refers to an -alkyl-O-heteroaryl substituent.

The term “nitrogen-containing heteroaryl” refers to a heteroaryl group having 1-4 ring nitrogen heteroatoms if monocyclic, 1-6 ring nitrogen heteroatoms if bicyclic, or 1-9 ring nitrogen heteroatoms if tricyclic.

The term “heterocycloalkyl” refers to a nonaromatic 3-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si, wherein the nonaromatic ring system is completely saturated. Heterocycloalkyl groups may be substituted or unsubstituted with one or more substituents. In one embodiment, 0, 1, 2, 3, or 4 atoms of each ring of a heterocycloalkyl group may be substituted by a substituent. Representative heterocycloalkyl groups include piperidinyl, piperazinyl, tetrahydropyranyl, morpholinyl, thiomorpholinyl, 1,3-dioxolane, tetrahydrofuranyl, tetrahydrothienyl, thiirenyl, and the like.

The term “heterocycloalkylalkyl” refers to an -alkyl-heterocycloalkyl substituent.

The term “alkylamino” refers to an amino substituent which is further substituted with one or two alkyl groups. The term “aminoalkyl” refers to an alkyl substituent which is further substituted with one or more amino groups. The term “hydroxyalkyl” or “hydroxylalkyl” refers to an alkyl substituent which is further substituted with one or more hydroxyl groups. The alkyl or aryl portion of alkylamino, aminoalkyl, mercaptoalkyl, hydroxyalkyl, mercaptoalkoxy, sulfonylalkyl, sulfonylaryl, alkylcarbonyl, and alkylcarbonylalkyl may be substituted or unsubstituted with one or more substituents.

The term “nucleobase” refers to nitrogen-containing biological compounds that form nucleosides. They include purine bases and pyrimidine bases. Five nucleobases—adenine (A), cytosine (C), guanine (G), thymine (T), and uracil (U)—are referred to as primary or canonical nucleobases. When a nucleobase is listed in a formula definition, it refers to that moiety covalently bonded to the recited formula.

The term “modified nucleobase” refers to derivatives of a nucleobase. Examples of modified nucleobases include, but are not limited to, xanthine, hypoxanthine,7-methylguanine, 5,6-dihydrouracil, 5-methylcytosine, 5-hydroxymethylcytosine, purine, 2,6-diaminopurine, and 6,8-diaminopurine. When a modified nucleobase is listed in a formula definition, it refers to that moiety covalently bonded to the recited formula.

The term “substituent” and “substituent group” means an atom or group that replaces the atom or group of a named parent compound. For example, a substituent of a modified nucleoside is an atom or group that differs from the atom or group found in a naturally occurring nucleoside (e.g., a modified 2′-substituent is any atom or group at the 2′-position of a nucleoside other than H or OH). Substituent groups can be protected or unprotected. Substituents may also be further substituted with other substituent groups and may be attached directly or via a linking group such as an alkyl or hydrocarbyl group to the parent compound. Similarly, as used herein, “substituent” in reference to a chemical functional group means an atom or group of atoms that differs from the atom or group of atoms normally present in the named functional group. In certain embodiments, substituents on any group (such as, for example, alkyl, alkenyl, alkynyl, aryl, aralkyl, heteroaryl, heteroaralkyl, cycloalkyl, heterocycloalkyl) can be at any atom of that group, wherein any group that can be substituted (such as, for example, alkyl, alkenyl, alkynyl, aryl, aralkyl, heteroaryl, heteroaralkyl, cycloalkyl, heterocycloalkyl) can be substituted or unsubstituted with one or more substituents (which may be the same or different), each replacing a hydrogen atom. Examples of suitable substituents include, but are not limited to alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, aryloxycarbonyl, heteroaryloxy, heteroaryloxycarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, amido, alkylaminosulfonyl, arylaminosulfonyl, dialkylaminosulfonyl, alkylsulfonylamino, arylsulfonylamino, imino, carboxamido, carbamido, carbamyl, thioureido, thiocyanato, sulfoamido, sulfonylalkyl, sulfonylaryl, mercaptoalkoxy, N-hydroxyamidinyl, or N′-aryl, N″-hydroxyamidinyl. In certain embodiments, substituents on any group include alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, thiocarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, or amido. In certain embodiments, substituents on any group include alkyl, halogen, haloalkyl, cyano, nitro, alkoxy, hydroxyl, hydroxylalkyl, carboxyl, formyl, alkylcarbonyl, alkoxycarbonyl, alkylcarbonyloxy, thio, mercapto, mercaptoalkyl, amino, aminoalkyl, dialkylamino, alkylcarbonylamino, alkylaminocarbonyl, or alkylamino.

The term “protecting group” or “protecting moiety” refers to a substituent that is commonly employed to block or protect a particular functionality while reacting other functional groups on the compound, a derivative thereof, or a conjugate thereof, and includes a nitrogen protecting group when attached to a nitrogen atom, or an oxygen protecting group when attached to an oxygen atom. Nitrogen and oxygen protecting groups are well known in the art and include those described in detail in Protecting Groups in Organic Synthesis, T. W. Greene and P. G. M. Wuts, 3rd edition, John Wiley & Sons, 1999, incorporated herein by reference.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein each R^{aa}, R^{bb}, and R^{cc} is independently alkyl, cycloalkyl, aryl, or heteroaryl, each of which may be substituted or unsubstituted with 1-3 independent R^{dd}, and each R^{dd} is independently alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, aryloxycarbonyl, heteroaryloxy, heteroaryloxycarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, amido, alkylaminosulfonyl, arylaminosulfonyl, dialkylaminosulfonyl, alkylsulfonylamino, arylsulfonylamino, imino, carbamido, carbamyl, thioureido, thiocyanato, sulfoamido, sulfonylalkyl, sulfonylaryl, or mercaptoalkoxy. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Amide nitrogen protecting groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}—, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), t-butyl carbonate (BOC), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, the protecting group or oxygen protecting group is a dimethoxytrityl group. In certain embodiments, the protecting group or oxygen protecting group is a 2-cyanoethyl 5′-O-(4,4′-dimethoxytrityl)thymidine-3′-O—(N,N-diisopropylamino)-phosphoramidite group.

In certain embodiments, the substituent present on a sulfur atom is a sulfur protecting group (also referred to as a thiol protecting group). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{cc})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesisz, T. W. Greene and P. G. M. Wuts, 3^{rd} edition, John Wiley & Sons, 1999, incorporated herein by reference.

The term “antisense oligonucleotide” or “antisense strand” means an oligonucleotide which includes a region that is complementary to a target nucleic acid.

The term “composition” or “pharmaceutical composition” means a mixture of substances suitable for administering to a subject. For example, a composition may comprise one or more compounds or salt thereof and a sterile aqueous solution.

The term “nucleic acid” refers to molecules composed of linked monomeric nucleotides or nucleosides. A nucleic acid includes, but is not limited to, ribonucleic acids (RNA), deoxyribonucleic acids (DNA), single-stranded nucleic acids, and double-stranded nucleic acids.

The term “nucleobase sequence” means the order of contiguous nucleobases in a nucleic acid or oligonucleotide independent of any sugar or internucleoside linkage.

The term “nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase.

The term “oligomeric compound” means a polymer of linked subunits. With reference to a protein, peptide, polypeptide, or antibody, “subunit” refers to an amino acid or peptide bond. With reference to an oligonucleotide, “subunit” refers to a nucleotide, nucleoside, nucleobase, or sugar, or a modified nucleotide, nucleoside, nucleobase, or sugar as provided herein.

The term “oligonucleotide” means a polymer of linked nucleosides (e.g., polynucleotide, nucleic acid, polymer of nucleotides), each of which can be modified or unmodified, independent from one another. Without limitation, an oligonucleotide may be comprised of ribonucleic acids (e.g., comprised of ribonucleosides), deoxyribonucleic acids (e.g., comprised of deoxyribonucleosides), modified nucleic acids (e.g., comprised of modified nucleobases, sugars, and/or phosphate groups), or a combination thereof. Examples of oligonucleotide compounds include single-stranded and double-stranded compounds, such as, oligonucleotides, antisense oligonucleotides, interfering RNA compounds (RNAi compounds), microRNA (miRNA) targeting oligonucleotides and miRNA mimics, occupancy-based compounds (e.g., mRNA processing or translation blocking compounds and splicing compounds). RNAi compounds include double-stranded compounds (e.g., short-interfering RNA (siRNA) and double-stranded RNA (dsRNA)) and single-stranded compounds (e.g., single-stranded siRNA (ssRNA), single-stranded RNAi (ssRNAi), short hairpin RNA (shRNA), and microRNA mimics) which work at least in part through the RNA-induced silencing complex (RISC) pathway resulting in sequence specific degradation and/or sequestration of a target nucleic acid through a process known as RNA interference (RNAi). The term “RNAi compound” is meant to be equivalent to other terms used to describe nucleic acid compounds that are capable of mediating sequence-specific RNA interference, for example, interfering RNA (iRNA), iRNA agent, RNAi agent, small interfering RNA, short interfering RNA, short interfering oligonucleotide, short interfering nucleic acid, short interfering modified oligonucleotide, chemically modified siRNA, and others. Additionally, the term “RNAi” is meant to be equivalent to other terms used to describe sequence-specific RNA interference.

The terms “target nucleic acid,” “target RNA,” and “nucleic acid target” all mean a nucleic acid capable of being targeted by compounds described herein.

The term “therapeutic compound” includes any pharmaceutical agent or compound that provides a therapeutic benefit to a subject. Therapeutic compounds include nucleic acids, oligomeric compounds, oligonucleotides, proteins, peptides, antibodies, small molecules, and other such agents.

“Target region” means a portion of a target nucleic acid to which one or more compounds is targeted.

“Targeting moiety” means a conjugate group that provides an enhanced affinity for a selected target, e.g., molecule, cell or cell type, compartment, e.g., a cellular or organ compartment, tissue, organ, or region of the body, as, e.g., compared to a compound absent such a moiety.

“Terminal group” means a chemical group or group of atoms that is covalently linked to a terminus of an oligonucleotide.

The term “conjugate group” means a group of atoms that is attached to an oligonucleotide. A conjugate group is optionally attached to an oligonucleotide through a linker. A conjugate group may, for example, alter the distribution, targeting, or half-life of a compound into which it is incorporated. Conjugate groups include lipids (or lipophilic moieties), ligands, and other targeting moieties, such as GalNAc moieties.

“Conjugate linker” means a group of atoms comprising at least one bond that connects a linked moiety to an oligonucleotide and/or other therapeutic agent.

The term “lipid” or “lipophilic moiety” refers to an aliphatic, cylic (such as alicyclic), or polycyclic (such as polyalicyclic) compound, such as a steroid (e.g., sterol) or a linear or branched aliphatic hydrocarbon. The term lipid includes cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl)lithocholic acid, O3-(oleoyl)cholenic acid, ibuprofen, naproxen, dimethoxytrityl, or phenoxazine. The term lipid includes a saturated or unsaturated C₄-C₃₀ hydrocarbon chain (e.g., C₄-C₃₀ alkyl or alkenyl). In certain embodiments, the lipophilic moiety contains a saturated or unsaturated C₆-C₁₈ hydrocarbon chain (e.g., a linear C₆-C₁₈ alkyl or alkenyl). In certain embodiments, the lipophilic moiety contains a saturated or unsaturated C₁₆ hydrocarbon chain (e.g., a linear C₁₆ alkyl or alkenyl). In certain embodiments, the lipophilic moiety contains a saturated or unsaturated Cis hydrocarbon chain (e.g., a linear Cis alkyl or alkenyl).

The term “ligand” refers to a substance that binds to or otherwise interacts with a protein, nucleic acid, or other biological molecule. In some embodiments, a ligand is a small molecule. In some embodiments, a ligand binds to a protein (e.g., a receptor). In certain embodiments, a ligand binds to an α₄β_{1/7} integrin receptor. In certain embodiments, a ligand binds to a receptor (e.g., an α₄β_{1/7} integrin, TrkB, CB₁ or NMDA receptor). In certain embodiments, a ligand binds to a CB₁ receptor. In certain embodiments, a ligand binds a Tropomyosin receptor B (TrkB) receptor. In certain embodiments, a ligand binds to a an α₄β_{1/7} integrin receptor. In certain embodiments, a ligand binds to an N-methyl-D-aspartate (NMDA) receptor.

In certain embodiments, a compound comprising a receptor (e.g., an α₄β_{1/7} integrin, TrkB, or CB₁ receptor) ligand selectively or preferentially targets a cell expressing that receptor compared to a cell not expressing that receptor (e.g., an α₄β_{1/7} integrin, TrkB, CB₁, or NMDA receptor). In certain embodiments, a compound comprising a receptor ligand (e.g., an α₄β_{1/7} integrin, TrkB, CB₁, or NMDA receptor) selectively or preferentially targets a cell expressing that receptor (e.g., an α₄β_{1/7} integrin, TrkB, CB₁, or NMDA receptor) compared to a compound not comprising that receptor ligand (e.g., an α₄β_{1/7} integrin, TrkB, CB₁, or NMDA receptor).

The term “α₄β_{1/7} integrin receptor” refers to heterodimeric integrin receptors formed by association of integrin alpha 4 and integrin beta 1 (i.e., the α₄β₁ integrin receptor) and integrin alpha 4 and integrin beta 7 (i.e., the α₄β₇ integrin receptor).

The term “Cannabinoid Receptor Type 1” or “CB₁” means the G protein-coupled receptor for cannabinoids. In humans, CB₁ is encoded by the CNR1 gene. CB₁ is also known as cannabinoid receptor 1.

In some embodiments, a nucleic acid is conjugated to a GalNAc moiety. GalNAc (N-acetylgalactosamine) is an amino sugar derivative of galactose. In some embodiments, a GalNAc moiety comprises the structure

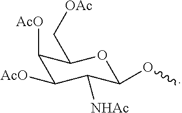

In some embodiments, a GalNAc moiety comprises the structure

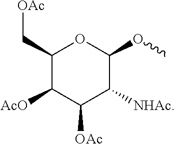

GalNAc moieties are targeting moieties that have an affinity for various tissues and cell receptors. In this way, GalNAc moieties can facilitate the targeting of cargo (e.g., nucleic acids) to such tissues and receptors. In some embodiments, a GalNAc moiety is useful for directing nucleic acids. In some embodiments, a GalNAc moiety directs a nucleic acid to a locality. In some embodiments, a GalNAc moiety targets tissues. In some embodiments, the tissue is liver. In some embodiments, a GalNAc moiety targets a cell receptor. In some embodiments, a cell receptor is an asialoglycoprotein receptor. In some embodiments, an asialoglycoprotein receptor on a hepatocyte.

The term “sense oligonucleotide” or “sense strand” means the strand of a double-stranded compound that includes a region that is substantially complementary to a region of the antisense strand of the double-stranded compound.

The terms “microRNA” and “miRNA,” as may be used interchangeably herein, refer to short (e.g., about 20 to about 24 nucleotides in length) non-coding ribonucleic acids (RNAs) that are involved in post-transcriptional regulation of gene expression in multicellular organisms by affecting both the stability and translation of mRNAs. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce a stem-loop precursor miRNA (pre-miRNA) approximately 70 nucleotides in length, which is further processed in the RNAi pathway. As part of this pathway, the pre-miRNA is cleaved by the cytoplasmic Dicer ribonuclease to generate the mature miRNA and antisense miRNA star (miRNA*) products. The mature miRNA is incorporated into an RNA-induced silencing complex (RISC), which recognizes target mRNAs through imperfect base pairing (i.e., partial complementarity) with the miRNA and most commonly results in translational inhibition or destabilization of the target mRNA. This mechanism is most often seen through the binding of the miRNA on the 3′ untranslated region (UTR) of the target mRNA, which can decrease gene expression by either inhibiting translation (for example, by blocking the access of ribosomes for translation) or directly causing degradation of the transcript. The term (i.e., miRNA) may be used herein to refer to any form of the subject miRNA (e.g., precursor, primary, and/or mature miRNA).

The terms “small interfering RNA,” “short interfering RNA,” and “siRNA,” as may be used interchangeably herein, refer to RNA molecules that present as non-coding double-stranded RNA (dsRNA) molecules of about 20 to about 24 nucleotides in length and are useful in RNA interference (RNAi). siRNA are often found with phosphorylated 5′ ends and hydroxylated 3′ ends, which 3′ ends typically have a 2-nucleotide overhang beyond the 5′ end of the anti-parallel strand (e.g., complementary strand of the dsRNA molecule). siRNA can interfere with the expression of specific genes through binding of target sequences (e.g., target nucleic acid sequences) to which they are complementary and promoting (e.g., facilitating, triggering, initiating) degradation of the mRNA, thereby preventing (e.g., inhibiting, silencing, interfering with) translation. After integration and separation into the RISC complex, siRNAs base-pair (e.g., full complementarity) to their target mRNA and cleave it, thereby preventing it from being used as a translation template. As discussed herein above, also part of the RNAi pathway, a miRNA-loaded RISC complex scans cytoplasmic mRNAs for potential complementarity (e.g., partial complementarity).

The term “ADAR recruiting molecule,” as may be used herein, refers to a nucleic acid that is configured to increase the concentration of Adenosine Deaminase Acting on Ribonucleic Acid (ADAR) enzyme in a locality around the nucleic acid. In some embodiments, an increased concentration is relative to the concentration in a given locality absent the ADAR recruiting molecule. In some embodiments, an ADAR recruiting molecule comprises a double-stranded RNA duplex.

The term “ADAR targeting molecule,” as may be used herein, refers to a nucleic acid that is configured to direct an ADAR molecule to a desirable location (e.g., locality). As used herein, the term “direct” refers to increasing the concentration of ADAR in the desirable location as compared to the concentration absent the ADAR targeting molecule. In some embodiments, the ADAR targeting molecule can be configured to control the desirable location by altering the sequence and/or properties of the nucleic acid (e.g., by modifications to the nucleobase, sugar, phosphate, or other component). In some embodiments, an ADAR targeting molecule comprises an ADAR recruiting molecule and a single-stranded guide nucleic acid. In some embodiments, an ADAR targeting molecule comprises a double-stranded RNA duplex and a single-stranded guide nucleic acid.

The term “single-stranded guide nucleic acid,” as may be used herein, refers to a nucleic acid of a single strand, which comprises a specific sequence that is at least partially complementary to a target sequence. In some embodiments, the target sequence is at, adjacent to, or in proximity to, a locality where it is desirable to modulate ADAR concentration. In some embodiments, the level of complementarity is sufficient to facilitate binding (e.g., annealing) of the single-stranded guide nucleic acid to the target sequence.

“Modified oligonucleotide” means an oligonucleotide, wherein at least one sugar, nucleobase, or internucleoside linkage is modified.

“Nucleobase sequence” means the order of contiguous nucleobases in a nucleic acid or oligonucleotide independent of any sugar or internucleoside linkage.

The term “oligomeric duplex” means a duplex formed by two oligomeric compounds having complementary nucleobase sequences. Each oligomeric compound of an oligomeric duplex may be referred to as a “duplexed oligomeric compound.” The oligonucleotides of each oligomeric compound of an oligomeric duplex may include non-complementary overhanging nucleosides. In some embodiments, the terms “duplexed oligomeric compound” and “modified oligonucleotide” are used interchangeably. In other embodiments, the terms “oligomeric duplex” and “compound” are used interchangeably.

“Phosphorothioate linkage” means a modified phosphate linkage in which one of the non-bridging oxygen atoms is replaced with a sulfur atom.

The terms “RNA interference compound,” “RNAi compound,” and/or “iRNA agent” mean a compound that acts, at least in part, through an RNA-induced silencing complex (RISC) pathway or Ago2, but not through RNase H, to modulate a target nucleic acid and/or protein encoded by a target nucleic acid. RNAi compounds include, but are not limited to, double-stranded siRNA, single-stranded siRNA, and microRNA, including microRNA mimics.

The compounds of the present disclosure may also contain unnatural proportions of atomic isotopes at one or more of the atoms that constitute such compounds. For example, the compounds may be radiolabeled with radioactive isotopes, such as for example tritium (3H), iodine-125 (¹²⁵I), or carbon-14 (¹⁴C). All isotopic variations of the compounds of the present disclosure, whether radioactive or not, are encompassed within the scope of the present disclosure.

The term “isotopic variant” refers to a therapeutic agent (e.g., a compound and/or modified oligonucleotide disclosed herein) that contains an unnatural proportion of an isotope at one or more of the atoms that constitute such a therapeutic agent. In certain embodiments, an “isotopic variant” of a therapeutic agent contains unnatural proportions of one or more isotopes, including, but not limited to, hydrogen (H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine 123 (¹²³I), iodine-125 (¹²⁵I), iodine-127 (¹²⁷I), iodine-129 (¹²⁹I), and iodine-131 (¹³¹I). In certain embodiments, an “isotopic variant” of a therapeutic agent contains unnatural proportions of one or more isotopes, including, but not limited to, hydrogen (H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P) sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine 123 (¹²³I), iodine-125 (¹²⁵I), iodine-127 (¹²⁷I), iodine-129 (¹²⁹I), and iodine-131 (¹³¹I).

It will be understood that, in a therapeutic agent (e.g., a compound and/or modified oligonucleotide disclosed herein), any hydrogen can be ²H, for example, or any carbon can be ¹³C, for example, or any nitrogen can be ¹⁵N, for example, or any oxygen can be ¹⁸O, for example, where feasible according to the judgment of one of skill. In certain embodiments, an “isotopic variant” of a therapeutic agent contains unnatural proportions of deuterium (D).

### DETAILED DESCRIPTION

In certain embodiments, described herein is a compound of Formula (I′), or a salt or prodrug thereof:

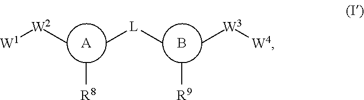

wherein:
A is a substituted tetrahydrofuranyl, substituted tetrahydropyranyl ring, or a modified sugar;
B is a substituted tetrahydrofuranyl, substituted tetrahydropyranyl ring, or a modified sugar;
W¹ and W⁴ are independently a modified or unmodified nucleoside, oligonucleotide, ligand, lipophilic moiety, or protecting group;
R⁸ and R⁹ are independently a substituted or unsubstituted heteroaryl;
L is a linker, a ligand, a lipophilic moiety, —Z¹—Y—Z²—, or a combination thereof;
W² and W³ are independently a bond, linker, substituted or unsubstituted alkylene, substituted or unsubstituted heteroalkylene, substituted or unsubstituted carbocyclylene, substituted or unsubstituted heterocyclylene, substituted or unsubstituted arylene, substituted or unsubstituted heteroarylene, —O—, —OP(O)O₂—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, S(O)₂NR^{A}—, —NR^{A}SO₂—, or a combination thereof;
each R^{A} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two R^{A} groups are joined to form with the atoms to which they are attached a substituted or unsubstituted heterocyclyl ring, or a substituted or unsubstituted heteroaryl ring;
Z¹ and Z² are independently a bond, substituted or unsubstituted alkylene, substituted or unsubstituted heteroalkylene, substituted or unsubstituted carbocyclylene, substituted or unsubstituted heterocyclylene, substituted or unsubstituted arylene, substituted or unsubstituted heteroarylene, —O—, —OP(O)O₂—, —N(R^{B})—, —S—, —C(═O)—, —C(═O)O—, —C(═O)NR^{B}—, —NR^{B}C(═O)—, —NR^{B}C(═O)R^{B}—, —C(═O)R^{B}—, —NR^{B}C(═O)O—, —NR^{B}C(═O)N(R^{B})—, —OC(═O)—, —OC(═O)O—, —OC(═O)N(R^{B})—, S(O)₂NR^{B}—, —NR^{B}SO₂—, or a combination thereof;
each R^{B} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two R^{B} groups are joined to form a substituted or unsubstituted heterocyclyl ring, or a substituted or unsubstituted heteroaryl ring;
Y is substituted or unsubstituted alkylene, substituted or unsubstituted heteroalkylene, substituted or unsubstituted carbocyclylene, substituted or unsubstituted heterocyclylene, substituted or unsubstituted arylene, substituted or unsubstituted heteroarylene, —O—, —OP(O)O₂—, —N(R^{C})—, —S—, —C(═O)—, —C(═O)O—, —C(═O)NR^{C}—, —NR^{C}C(═O)—, —NR^{C}C(═O)R^{C}—, —C(═O)R^{C}—, —NR^{C}C(═O)O—, —NR^{C}C(═O)N(R^{C})—, —OC(═O)—, —OC(═O)O—, —OC(═O)N(R^{C})—, —S(O)₂NR^{C}—, —NR^{C}SO₂—, or a combination thereof; and
each R^{C} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group, or two R^{C} groups are joined to form a substituted or unsubstituted heterocyclyl ring, or a substituted or unsubstituted heteroaryl ring.

In certain embodiments, the compound of Formula (I′) is a compound of Formula (VIII), or a salt or prodrug thereof:

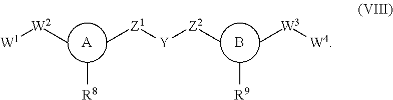

The present disclosure also provides compound of Formula (I):

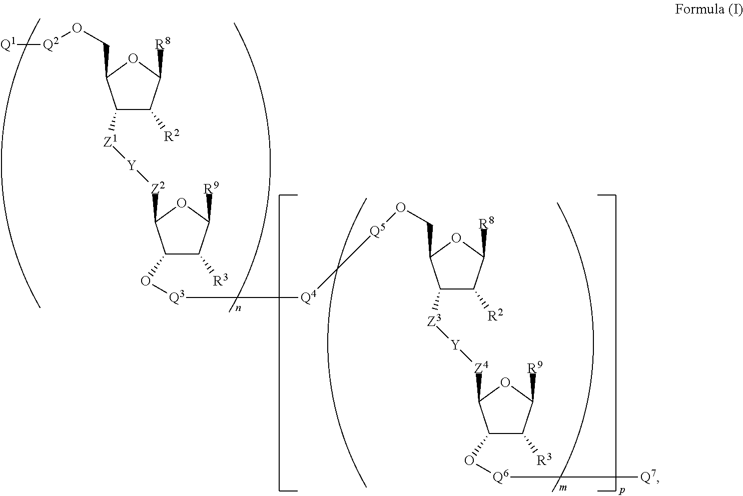

wherein:
Q¹ is —H, —OR⁴, a ligand, or a lipid;
each Q² is independently a bond,

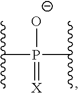

a ligand, a linker, or a lipid;
each Q³ is independently a bond,

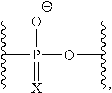

a ligand, a linker, or a lipid;
each Q⁴ is independently a bond, —R¹⁰O—, a ligand, a linker, or a lipid;
each Q⁵ is independently a bond,

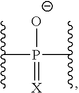

a ligand, a linker, or a lipid;
each Q⁶ is independently a bond,

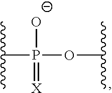

a ligand, a linker, or a lipid;
Q⁷ is —H, —R⁵, a ligand, or a lipid;
each Y is independently substituted or unsubstituted alkylene, substituted or unsubstituted heteroalkylene, substituted or unsubstituted carbocyclylene, substituted or unsubstituted heterocyclylene, substituted or unsubstituted arylene, substituted or unsubstituted heteroarylene, —O—, —OP(O)O₂—, —N(R^{C})—, —S—, —C(═O)—, —C(═O)O—, —C(═O)NR^{C}—, —NR^{C}C(═O)—, —NR^{C}C(═O)R^{C}—, —C(═O)R^{C}—, —NR^{C}C(═O)O—, —NR^{C}C(═O)N(R^{C})—, —OC(═O)—, —OC(═O)O—, —OC(═O)N(R^{C})—, —S(O)₂NR^{C}—, —NR^{C}SO₂—, or a combination thereof;
each R^{C} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two R^{C} groups are joined to form a substituted or unsubstituted heterocyclyl ring, or a substituted or unsubstituted heteroaryl ring;
each R² is independently —H, halogen, substituted or unsubstituted alkyl, substituted or unsubstituted alkenyl, substituted or unsubstituted alkynyl, —OR⁶, —N(R⁶), or —SR⁶.
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring;
each R¹⁰ is independently an oligonucleotide;
each X is independently O or S;
each instance of Z¹, Z², Z³, or Z⁴ is independently a bond, C₁-C₆ alkylene, or C₂-C₆ alkenylene;
n is 1, 2, 3, 4, 5, 6, 7, 8, 9, or 10;
each m is independently 1, 2, 3, 4, 5, 6, 7, 8, 9, or 10; and
p is 0, 1, 2, 3, 4, 5, 6, 7, 8, 9, or 10;
   or a salt or prodrug thereof.

Further provided here in are compounds of Formula (II):

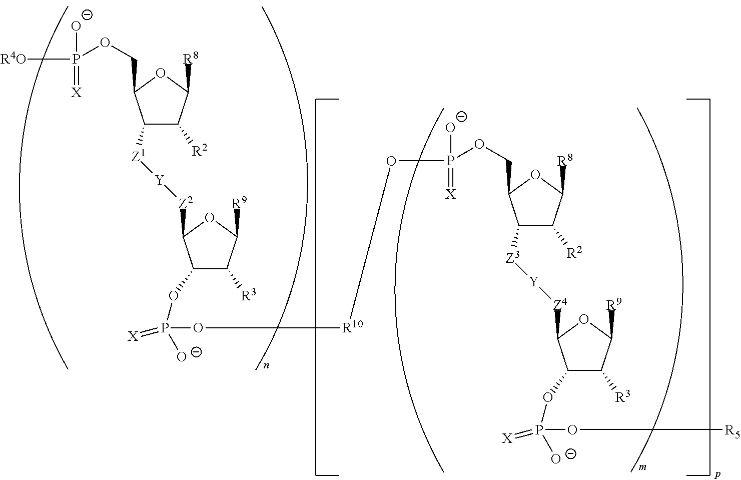

wherein:
each Y is independently substituted or unsubstituted alkylene, substituted or unsubstituted heteroalkylene, substituted or unsubstituted carbocyclylene, substituted or unsubstituted heterocyclylene, substituted or unsubstituted arylene, substituted or unsubstituted heteroarylene, —O—, —OP(O)O₂—, —N(R^{C})—, —S—, —C(═O)—, —C(═O)O—, —C(═O)NR^{C}—, —NR^{C}C(═O)—, —NR^{C}C(═O)R^{C}—, —C(═O)R^{C}—, —NR^{C}C(═O)O—, —NR^{C}C(═O)N(R^{C})—, —OC(═O)—, —OC(═O)O—, —OC(═O)N(R^{C})—, —S(O)₂NR^{C}—, —NR^{C}SO₂—, or a combination thereof;
each R^{C} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two R^{C} groups are joined to form a substituted or unsubstituted heterocyclyl ring, or a substituted or unsubstituted heteroaryl ring;
each R² is independently —H, halogen, substituted or unsubstituted alkyl, substituted or unsubstituted alkenyl, substituted or unsubstituted alkynyl, —OR⁶, —N(R⁶), or —SR⁶.
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl;
each R⁹ is independently a substituted or unsubstituted heteroaryl;
each R¹⁰ is independently an oligonucleotide;
each X is independently O or S;
each instance of Z¹, Z², Z³, or Z⁴ is independently a bond, C₁-C₆ alkylene, or C₂-C₆ alkenylene;
n is 1, 2, 3, 4, 5, 6, 7, 8, 9, or 10;
each m is independently 1, 2, 3, 4, 5, 6, 7, 8, 9, or 10; and
p is 0, 1, 2, 3, 4, 5, 6, 7, 8, 9, or 10;
   or a salt or prodrug thereof.

In certain embodiments, the compound of Formula (II) is a salt according to the following chemical structure:

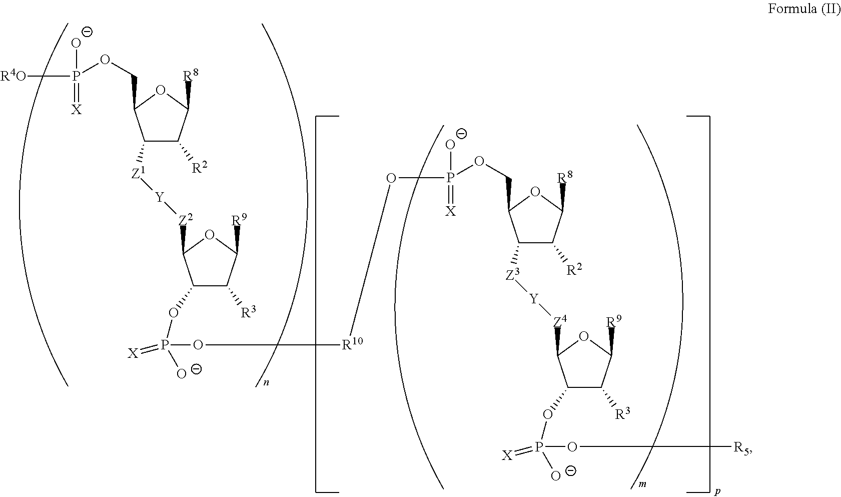

wherein:
each Y is independently substituted or unsubstituted alkylene, substituted or unsubstituted heteroalkylene, substituted or unsubstituted carbocyclylene, substituted or unsubstituted heterocyclylene, substituted or unsubstituted arylene, substituted or unsubstituted heteroarylene, —O—, —OP(O)O₂—, —N(R^{C})—, —S—, —C(═O)—, —C(═O)O—, —C(═O)NR^{C}—, —NR^{C}C(═O)—, —NR^{C}C(═O)R^{C}—, —C(═O)R^{C}—, —NR^{C}C(═O)O—, —NR^{C}C(═O)N(R^{C})—, —OC(═O)—, —OC(═O)O—, —OC(═O)N(R^{C})—, —S(O)₂NR^{C}—, —NR^{C}SO₂—, or a combination thereof;
each R^{C} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two R^{C} groups are joined to form a substituted or unsubstituted heterocyclyl ring, or a substituted or unsubstituted heteroaryl ring;
each R² is independently —H, halogen, substituted or unsubstituted alkyl, substituted or unsubstituted alkenyl, substituted or unsubstituted alkynyl, —OR⁶, —N(R⁶), or —SR⁶.
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl;
each R⁹ is independently a substituted or unsubstituted heteroaryl;
each R¹⁰ is independently an oligonucleotide;
each X is independently O or S;
each instance of Z¹, Z², Z³, or Z⁴ is independently a bond, C₁-C₆ alkylene, or C₂-C₆ alkenylene;
n is 1, 2, 3, 4, 5, 6, 7, 8, 9, or 10;
each m is independently 1, 2, 3, 4, 5, 6, 7, 8, 9, or 10; and
p is 0, 1, 2, 3, 4, 5, 6, 7, 8, 9, or 10;
   or a salt or prodrug thereof.

In certain embodiments, the salt is a potassium salt or sodium salt. In certain embodiments, the salt is a potassium salt. In certain embodiments, the salt is a sodium salt.

In certain embodiments, the compound of Formula (II) or the salt or prodrug thereof, is of the Formula (II-a):

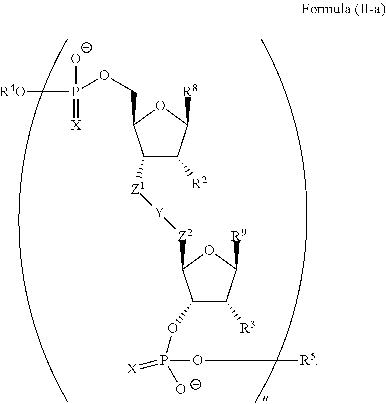

In certain embodiments, Z¹ is a bond. In certain embodiments, Z¹ is C₁-C₆ alkylene. In certain embodiments, Z¹ is C₂-C₆ alkenylene. In certain embodiments, Z¹ is —CH═CH—. In certain embodiments, Z¹ is —CH₂—. In certain embodiments, Z¹ is —CH₂CH₂—.

In certain embodiments, Z² is a bond. In certain embodiments, Z² is C₁-C₆ alkylene. In certain embodiments, Z² is C₂-C₆ alkenylene. In certain embodiments, Z² is —CH═CH—. In certain embodiments, Z² is —CH₂—. In certain embodiments, Z² is —CH₂CH₂—.

In certain embodiments, Z³ is a bond. In certain embodiments, Z³ is C₁-C₆ alkylene. In certain embodiments, Z³ is C₂-C₆ alkenylene. In certain embodiments, Z³ is —CH═CH—. In certain embodiments, Z³ is —CH₂—. In certain embodiments, Z³ is —CH₂CH₂—.

In certain embodiments, Z⁴ is a bond. In certain embodiments, Z⁴ is C₁-C₆ alkylene. In certain embodiments, Z⁴ is C₂-C₆ alkenylene. In certain embodiments, Z⁴ is —CH═CH—. In certain embodiments, Z⁴ is —CH₂—. In certain embodiments, Z⁴ is —CH₂CH₂—.

In certain embodiments, the compound of Formula (II) or the salt or prodrug thereof, is of the Formula (II-b):

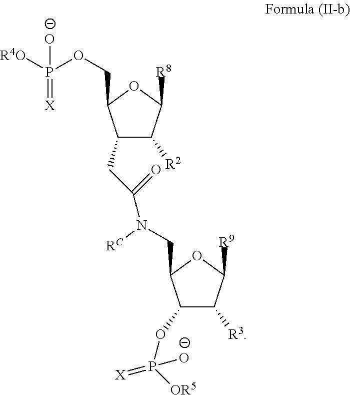

In some embodiments, R⁴ and R⁵ each comprise an oligonucleotide. In some embodiments, R⁴ and R⁵ are joined together to form a single oligonucleotide. In some embodiments, R⁴ comprises an oligonucleotide and R⁵ comprises a protecting group. In some embodiments, R⁴ comprises a protecting group and R⁵ comprises an oligonucleotide. In some embodiments, R⁴ and R⁵ each comprise a protecting group. In some embodiments, one or both of the oligonucleotides is attached at its 5′ end. In some embodiments, one or both of the oligonucleotides are attached at its 3′ end. In some embodiments, one or both of the oligonucleotides are attached at an internal position on the oligonucleotide. In certain embodiments, the internal position is an internucleoside linkage.

In certain embodiments, an oligonucleotide as disclosed herein is an oligonucleotide of Formula (IX):

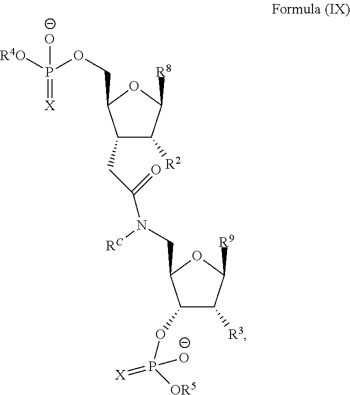

wherein:
R^{C} is —H, substituted or unsubstituted alkyl, substituted or unsubstituted alkenyl, substituted or unsubstituted alkynyl, substituted or unsubstituted heteroalkyl, substituted or unsubstituted aryl, or substituted or unsubstituted heteroaryl;
each R² is independently —H, halogen, substituted or unsubstituted alkyl, substituted or unsubstituted alkenyl, substituted or unsubstituted alkynyl, —OR⁶, —N(R⁶), or —SR⁶.
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, or a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

In certain embodiments, an oligonucleotide as disclosed herein is an oligonucleotide of Formula (X):

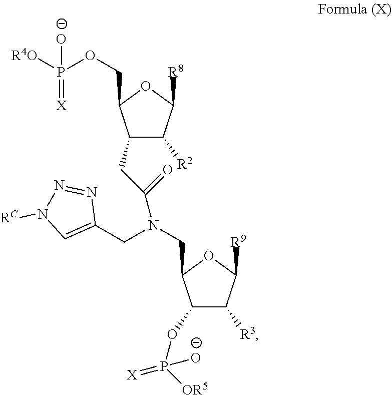

wherein:
R^{C} is —H, substituted or unsubstituted alkyl, substituted or unsubstituted alkenyl, substituted or unsubstituted alkynyl, substituted or unsubstituted heteroalkyl, substituted or unsubstituted aryl, or substituted or unsubstituted heteroaryl;
each R² is independently —H, halogen, substituted or unsubstituted alkyl, substituted or unsubstituted alkenyl, substituted or unsubstituted alkynyl, —OR⁶, —N(R⁶), or —SR⁶.
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

In certain embodiments, an oligonucleotide as disclosed herein is an oligonucleotide of Formula (XI):

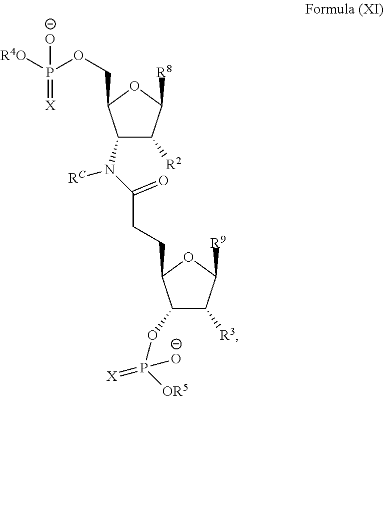

wherein:
R^{C} is —H, substituted or unsubstituted alkyl, substituted or unsubstituted alkenyl, substituted or unsubstituted alkynyl, substituted or unsubstituted heteroalkyl, substituted or unsubstituted aryl, or substituted or unsubstituted heteroaryl;
each R² is independently —H, halogen, substituted or unsubstituted alkyl, substituted or unsubstituted alkenyl, substituted or unsubstituted alkynyl, —OR⁶, —N(R⁶), or —SR⁶.
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

In certain embodiments, an oligonucleotide as disclosed herein is an oligonucleotide of Formula (XI-a):

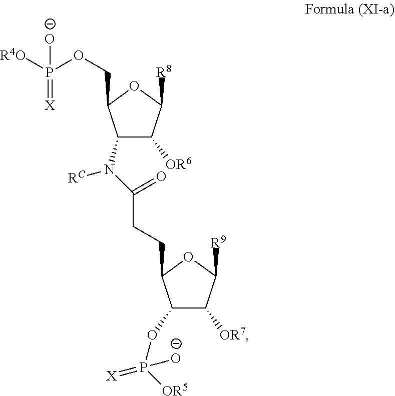

wherein:
R^{C} is —H, substituted or unsubstituted alkyl, substituted or unsubstituted alkenyl, substituted or unsubstituted alkynyl, substituted or unsubstituted heteroalkyl, substituted or unsubstituted aryl, or substituted or unsubstituted heteroaryl;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
   or a salt or prodrug thereof

In certain embodiments, an oligonucleotide as disclosed herein is an oligonucleotide of Formula (XI-b):

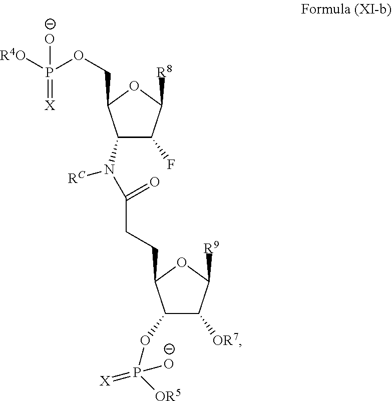

wherein:
R^{C} is —H, substituted or unsubstituted alkyl, substituted or unsubstituted alkenyl, substituted or unsubstituted alkynyl, substituted or unsubstituted heteroalkyl, substituted or unsubstituted aryl, or substituted or unsubstituted heteroaryl;
R⁴ and R⁵ are independently an oligonucleotide, a protecting group, or R⁴ and R⁵ are joined together to form a single oligonucleotide;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
   or a salt or prodrug thereof.

In certain embodiments, an oligonucleotide as disclosed herein is an oligonucleotide of Formula (XI-c):

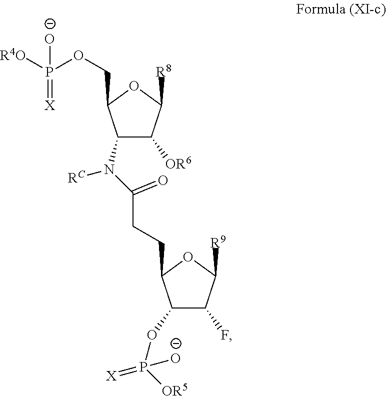

wherein:
R^{C} is —H, substituted or unsubstituted alkyl, substituted or unsubstituted alkenyl, substituted or unsubstituted alkynyl, substituted or unsubstituted heteroalkyl, substituted or unsubstituted aryl, or substituted or unsubstituted heteroaryl;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
   or a salt or prodrug thereof.

In certain embodiments, an oligonucleotide as disclosed herein is an oligonucleotide of Formula (XII):

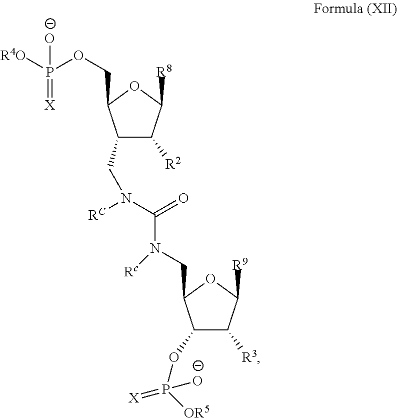

wherein:
each instance of R^{C} is independently —H, substituted or unsubstituted alkyl, substituted or unsubstituted alkenyl, substituted or unsubstituted alkynyl, substituted or unsubstituted heteroalkyl, substituted or unsubstituted aryl, or substituted or unsubstituted heteroaryl;
each R² is independently —H, halogen, substituted or unsubstituted alkyl, substituted or unsubstituted alkenyl, substituted or unsubstituted alkynyl, —OR⁶, —N(R⁶), or —SR⁶.
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

In certain embodiments, an oligonucleotide as disclosed herein is an oligonucleotide of Formula (XIII):

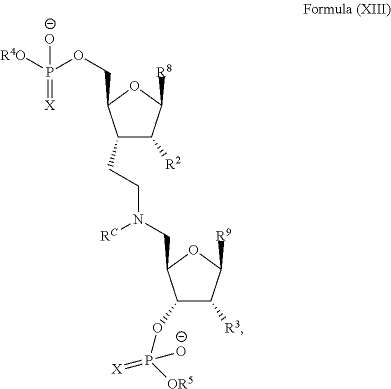

wherein:
R^{C} is —H, substituted or unsubstituted alkyl, substituted or unsubstituted alkenyl, substituted or unsubstituted alkynyl, substituted or unsubstituted heteroalkyl, substituted or unsubstituted aryl, or substituted or unsubstituted heteroaryl;
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
   or a salt or prodrug thereof.

In certain embodiments, an oligonucleotide as disclosed herein is an oligonucleotide of Formula (XIV):

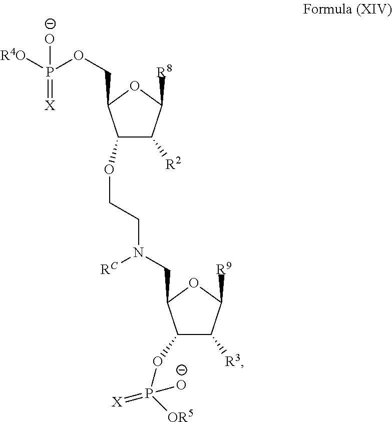

wherein:
R^{C} is —H, substituted or unsubstituted alkyl, substituted or unsubstituted alkenyl, substituted or unsubstituted alkynyl, substituted or unsubstituted heteroalkyl, substituted or unsubstituted aryl, or substituted or unsubstituted heteroaryl;
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

In certain embodiments, an oligonucleotide as disclosed herein is an oligonucleotide of Formula (IX-a):

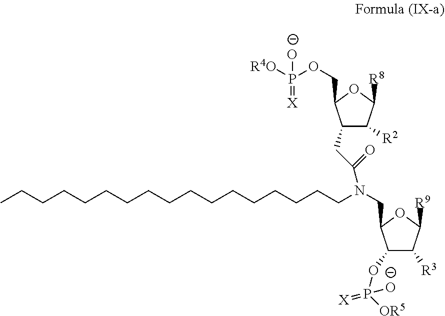

wherein:
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

In certain embodiments, an oligonucleotide as disclosed herein is an oligonucleotide of Formula (IX-b):

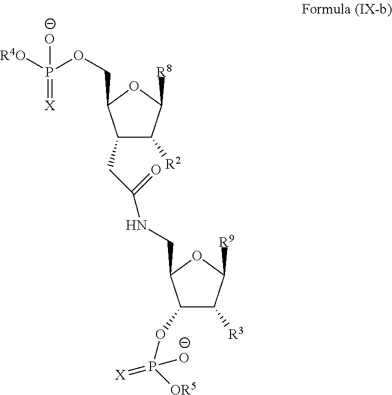

wherein:
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

In certain embodiments, an oligonucleotide as disclosed herein is an oligonucleotide of Formula (IX-c):

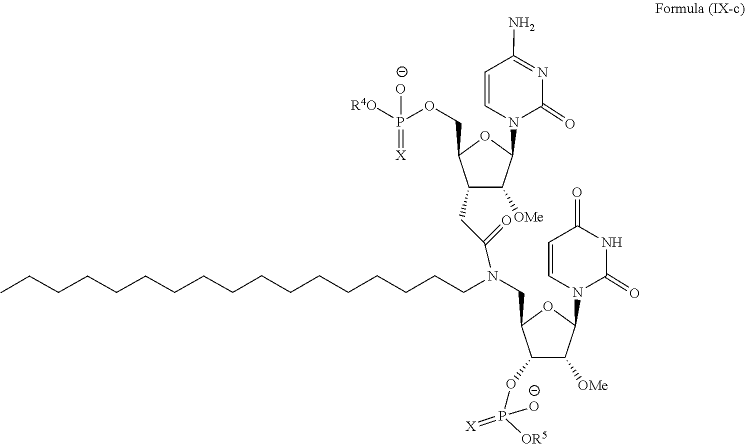

wherein:
R⁴ and R⁵ are independently an oligonucleotide, a protecting group, or R⁴ and R⁵ are joined together to form a single oligonucleotide; and
each X is independently O or S;
or a salt or prodrug thereof.

In certain embodiments, an oligonucleotide as disclosed herein is an oligonucleotide of Formula (IX-d):

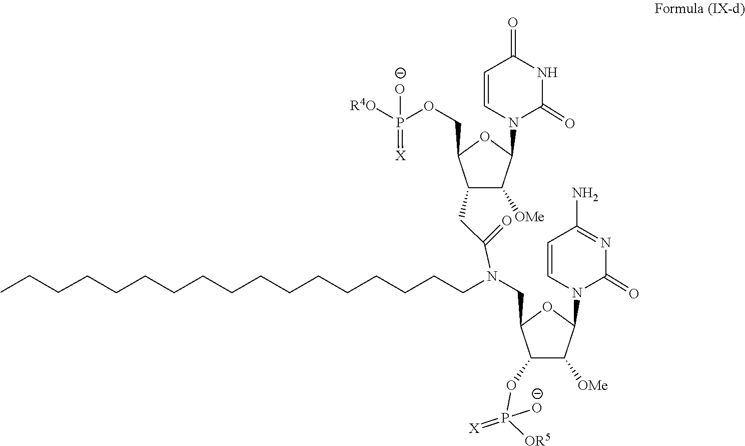

wherein:
R⁴ and R⁵ are independently an oligonucleotide, a protecting group, or R⁴ and R⁵ are joined together to form a single oligonucleotide; and
each X is independently O or S;
or a salt or prodrug thereof.

In certain embodiments, an oligonucleotide as disclosed herein is an oligonucleotide of Formula (IX-e):

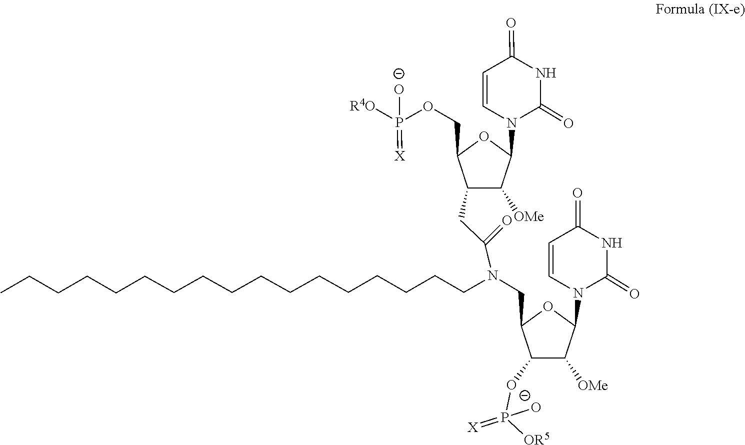

wherein:
R⁴ and R⁵ are independently an oligonucleotide, a protecting group, or R⁴ and R⁵ are joined together to form a single oligonucleotide; and
each X is independently O or S;
   or a salt or prodrug thereof.

In certain embodiments, an oligonucleotide as disclosed herein is an oligonucleotide of Formula (X-a):

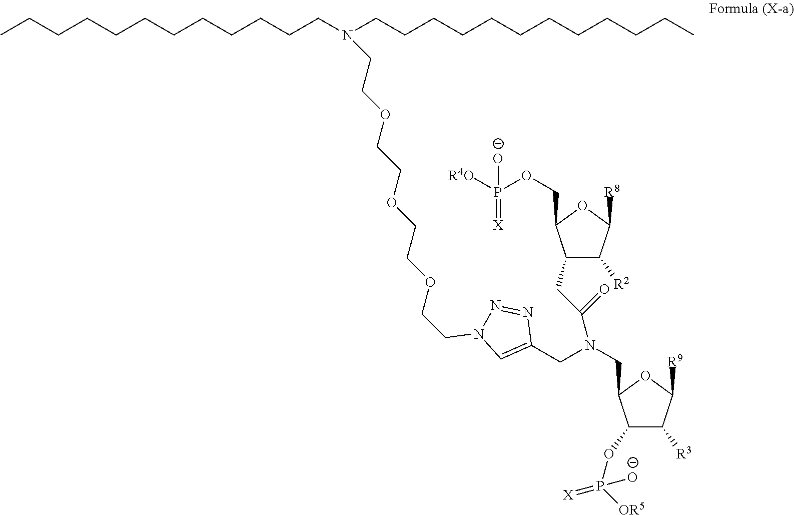

wherein:
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

In certain embodiments, an oligonucleotide as disclosed herein is an oligonucleotide of Formula (XV):

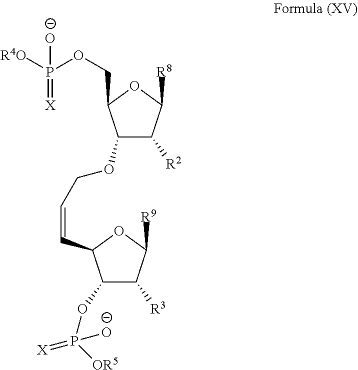

wherein:
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

In certain embodiments, an oligonucleotide as disclosed herein is an oligonucleotide of Formula (XVI):

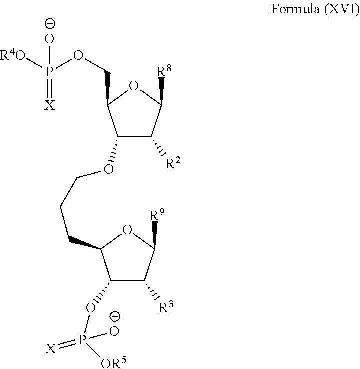

wherein:
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

In certain embodiments, an oligonucleotide as disclosed herein is an oligonucleotide of Formula (XVII):

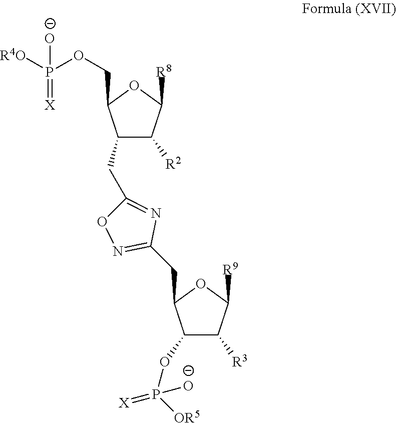

wherein:
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

In certain embodiments, an oligonucleotide as disclosed herein is an oligonucleotide of Formula (XVIII):

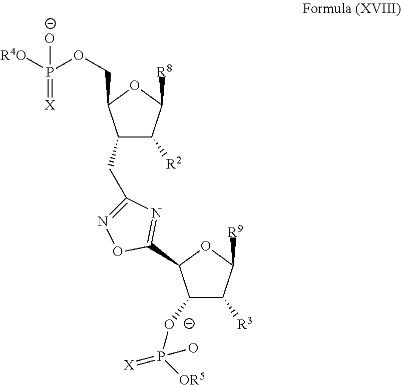

wherein:
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

In certain embodiments, the compound of Formula (II) is of the formula:

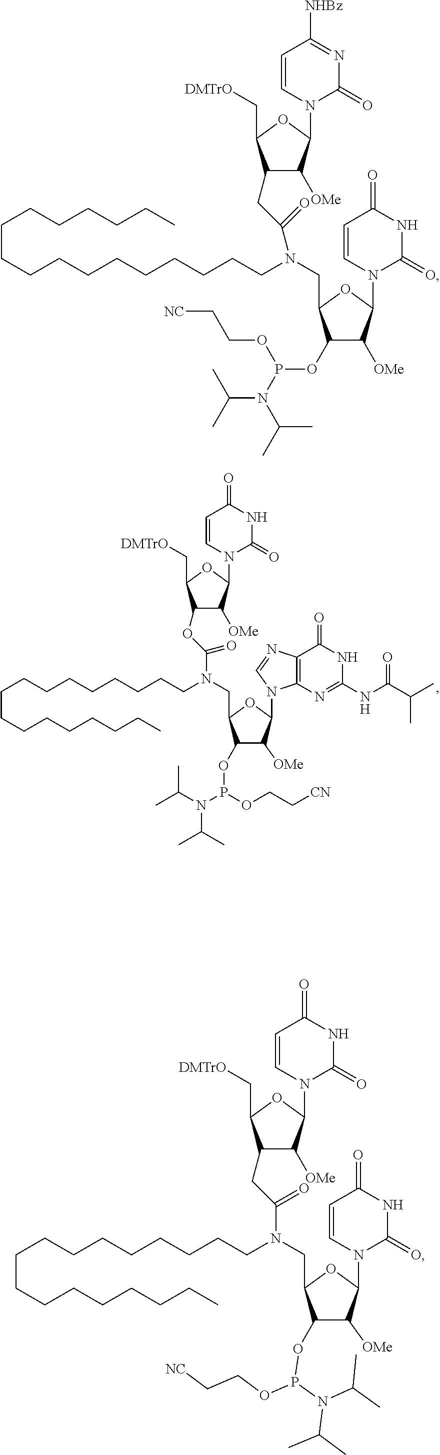

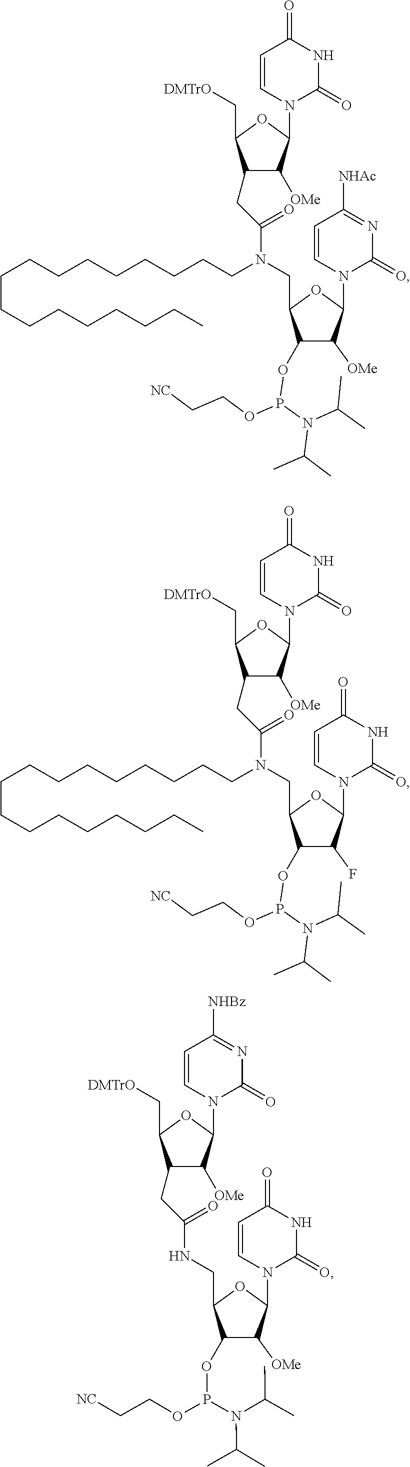

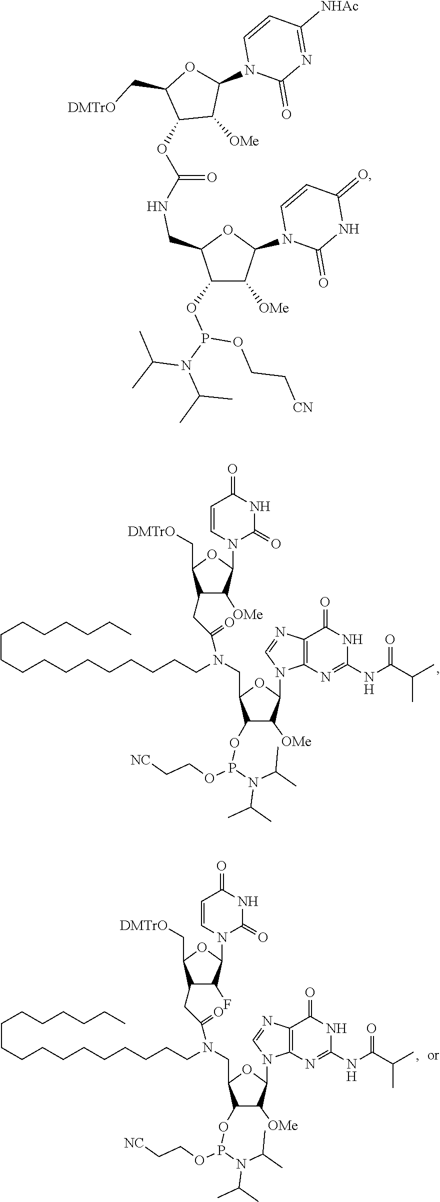

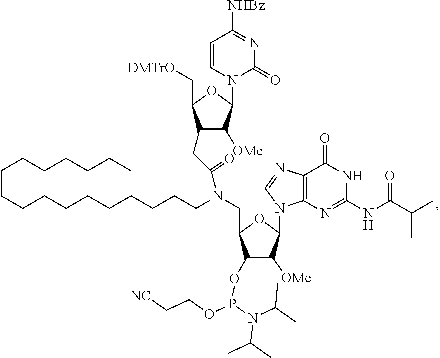

or a salt thereof.

Also provided herein are oligonucleotides of Formula (VI) or the salt or prodrug thereof, is of the Formula (VI):

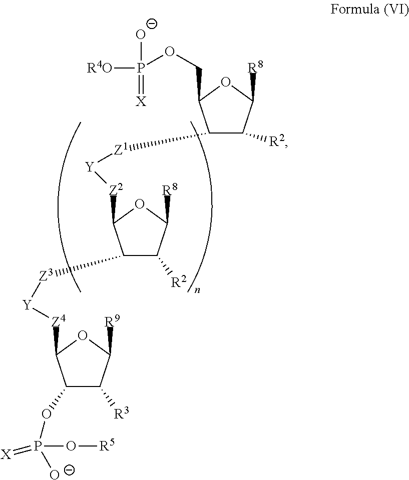

wherein:
   each Y is independently substituted or unsubstituted alkylene, substituted or unsubstituted heteroalkylene, substituted or unsubstituted carbocyclylene, substituted or unsubstituted heterocyclylene, substituted or unsubstituted arylene, substituted or unsubstituted heteroarylene, —O—, —OP(O)O₂—, —N(R^{C})—, —S—, —C(═O)—, —C(═O)O—, —C(═O)NR^{C}—, —NR^{C}C(═O)—, —NR^{C}C(═O)R^{C}—, —C(═O)R^{C}—, —NR^{C}C(═O)O—, —NR^{C}C(═O)N(R^{C})—, —OC(═O)—, —OC(═O)O—, —OC(═O)N(R^{C})—, —S(O)₂NR^{C}—, —NR^{C}SO₂—, or a combination thereof;
each R^{C} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two R^{C} groups are joined to form a substituted or unsubstituted heterocyclyl ring, or a substituted or unsubstituted heteroaryl ring;
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are each independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently an substituted or unsubstituted heteroaryl;
each R⁹ is independently an substituted or unsubstituted heteroaryl;
each X is independently O or S;
   each instance of Z¹, Z², Z³, or Z⁴ is independently a bond, C₁-C₆ alkylene, or C₂-C₆ alkenylene;
n is 1, 2, 3, 4, 5, 6, 7, 8, 9, or 10;
   or a salt or prodrug thereof.

In certain embodiments, R⁴ and R⁵ are each independently an oligonucleotide or a protecting group.

Also provided herein are compounds of Formula (VII):

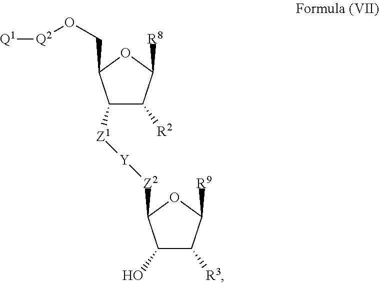

wherein:
Y is substituted or unsubstituted alkylene, substituted or unsubstituted heteroalkylene, substituted or unsubstituted carbocyclylene, substituted or unsubstituted heterocyclylene, substituted or unsubstituted arylene, substituted or unsubstituted heteroarylene, —O—, —OP(O)O₂—, —N(R^{C})—, —S—, —C(═O)—, —C(═O)O—, —C(═O)NR^{C}—, —NR^{C}C(═O)—, —NR^{C}C(═O)R^{C}—, —C(═O)R^{C}—, —NR^{C}C(═O)O—, —NR^{c}C(═O)N(R^{C})—, —OC(═O)—, —OC(═O)O—, —OC(═O)N(R^{C})—, —S(O)₂NR^{C}—, —NR^{C}SO₂—, or a combination thereof;
Q¹ is —H, —OR⁴, a ligand, a linker, or a lipid;
Q² is independently a bond,

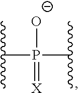

a ligand, a linker, or a lipid;
each R^{C} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two R^{C} groups are joined to form a substituted or unsubstituted heterocyclyl ring, or a substituted or unsubstituted heteroaryl ring;
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl;
each R⁹ is independently a substituted or unsubstituted heteroaryl;
each instance of Z¹, Z², Z³, or Z⁴, when present, is independently a bond, C₁-C₆ alkylene, or C₂-C₆ alkenylene;
n is 1, 2, 3, 4, 5, 6, 7, 8, 9, or 10;
or a salt or prodrug thereof.

In certain embodiments, a compound of Formula (I′) is of the formula:

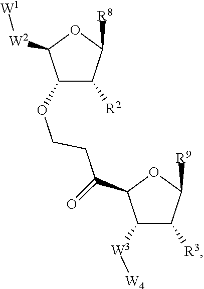

wherein W¹, W², W³, W⁴, R², R³, R⁸, and R⁹ are as defined herein. In certain embodiments, a compound of Formula (I′) is of the formula:

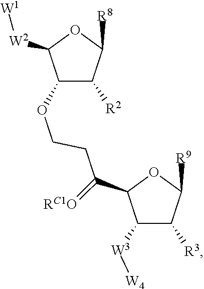

wherein R^{C1} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, or an oxygen protecting group. And wherein W¹, W², W³, W⁴, R², R³, R⁸, and R⁹ are as defined herein. In certain embodiments, a compound of Formula (I′) is of the formula:

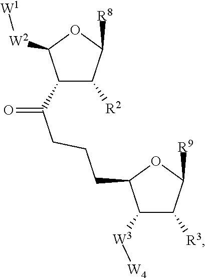

wherein W¹, W², W³, W⁴, R², R³, R⁸, and R⁹ are as defined herein. In certain embodiments, a compound of Formula (I′) is of the formula:

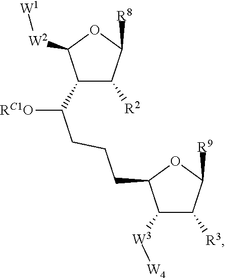

wherein R^{C1} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, or an oxygen protecting group, and wherein W¹, W², W³, W⁴, R², R³, R⁸, and R⁹ are as defined herein. In certain embodiments, a compound of Formula (I′) is of the formula:

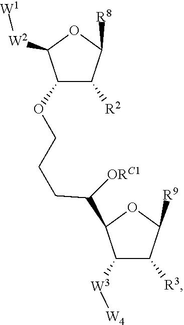

wherein W¹, W², W³, W⁴, R², R³, R⁸, and R⁹ are as defined herein. In certain embodiments, a compound of Formula (I′) is of the formula:

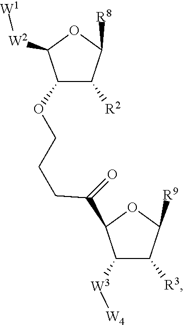

wherein W¹, W², W³, W⁴, R², R³, R⁸, and R⁹ are as defined herein. In certain embodiments, a compound of Formula (I′) is of the formula:

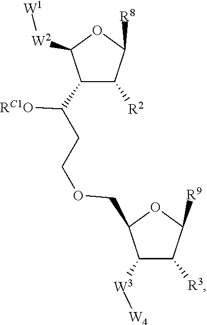

wherein W¹, W², W³, W⁴, R², R³, R⁸, and R⁹ are as defined herein. In certain embodiments, a compound of Formula (I′) is of the formula:

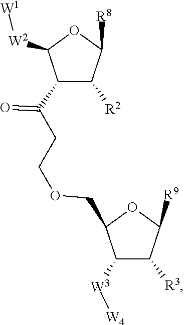

wherein W¹, W², W³, W⁴, R², R³, R⁸, and R⁹ are as defined herein. In certain embodiments, a compound of Formula (I′) is of the formula:

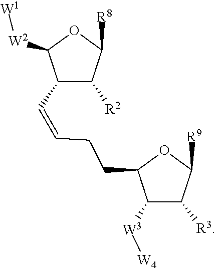

Wherein W¹, W², W³, W⁴, R², R³, R⁸, and R⁹ are as defined herein. In certain embodiments, a compound of Formula (I′) is of the formula:

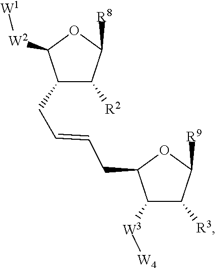

wherein W¹, W², W³, W⁴, R², R³, R⁸, and R⁹ are as defined herein. In certain embodiments, a compound of Formula (I′) is of the formula:

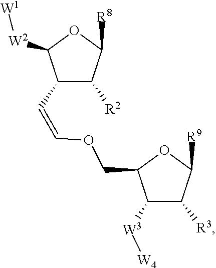

wherein W¹, W², W³, W⁴, R², R³, R⁸, and R⁹ are as defined herein. In certain embodiments, a compound of Formula (I′) is of the formula:

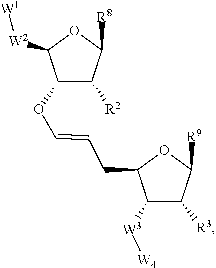

wherein W¹, W², W³, W⁴, R², R³, R⁸, and R⁹ are as defined herein. In certain embodiments, a compound of Formula (I′) is of the formula:

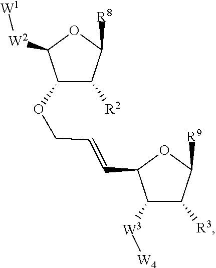

wherein W¹, W², W³, W⁴, R², R³, R⁸, and R⁹ are as defined herein. In certain embodiments, a compound of Formula (I′) is of the formula:

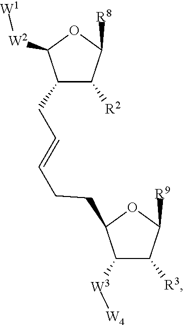

wherein W¹, W², W³, W⁴, R², R³, R⁸, and R⁹ are as defined herein. In certain embodiments, a compound of Formula (I′) is of the formula:

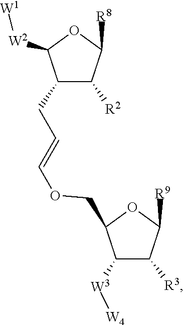

wherein W¹, W², W³, W⁴, R², R³, R⁸, and R⁹ are as defined herein. In certain embodiments, a compound of Formula (I′) is of the formula:

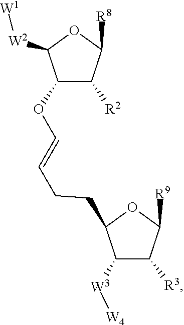

wherein W¹, W², W³, W⁴, R², R³, R⁸, and R⁹ are as defined herein. In certain embodiments, a compound of Formula (I′) is of the formula:

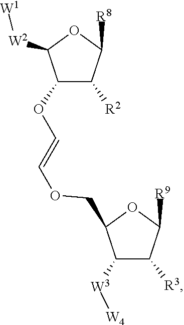

wherein W¹, W², W³, W⁴, R², R³, R⁸, and R⁹ are as defined herein. In certain embodiments, a compound of Formula (I′) is of the formula:

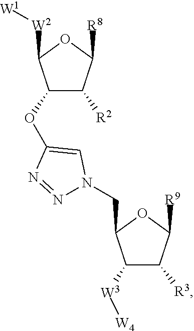

wherein W¹, W², W³, W⁴, R², R³, R⁸, and R⁹ are as defined herein. In certain embodiments, a compound of Formula (I′) is of the formula:

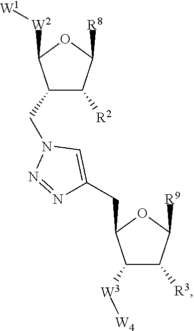

wherein W¹, W², W³, W⁴, R², R³, R⁸, and R⁹ are as defined herein. In certain embodiments, a compound of Formula (I′) is of the formula:

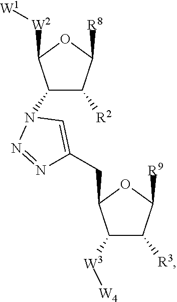

wherein W¹, W², W³, W⁴, R², R³, R⁸, and R⁹ are as defined herein. In certain embodiments, a compound of Formula (I′) is of the formula:

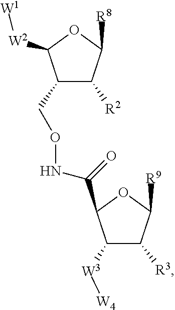

wherein W¹, W², W³, W⁴, R², R³, R⁸, and R⁹ are as defined herein. In certain embodiments, a compound of Formula (I′) is of the formula:

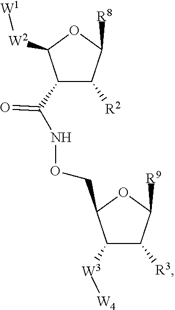

wherein W¹, W², W³, W⁴, R², R³, R⁸, and R⁹ are as defined herein. In certain embodiments, a compound of Formula (I′) is of the formula:

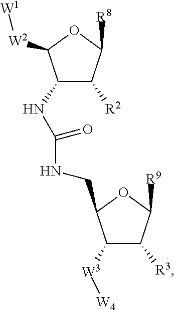

wherein W¹, W², W³, W⁴, R², R³, R⁸, and R⁹ are as defined herein. In certain embodiments, a compound of Formula (I′) is of the formula:

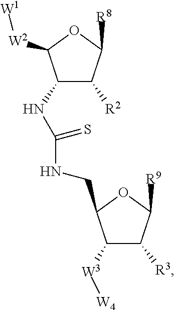

wherein W¹, W², W³, W⁴, R², R³, R⁸, and R⁹ are as defined herein. In certain embodiments, a compound of Formula (I′) is of the formula:

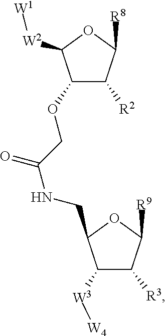

wherein W¹, W², W³, W⁴, R², R³, R⁸, and R⁹ are as defined herein. In certain embodiments, a compound of Formula (I′) is of the formula:

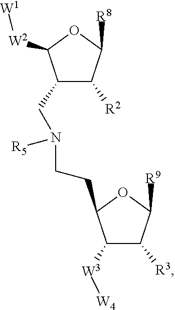

wherein W¹, W², W³, W⁴, R², R³, R⁸, and R⁹ are as defined herein. In certain embodiments, a compound of Formula (I′) is of the formula:

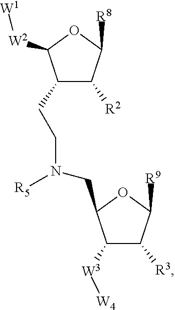

wherein W¹, W², W³, W⁴, R², R³, R⁸, and R⁹ are as defined herein. In certain embodiments, a compound of Formula (I′) is of the formula:

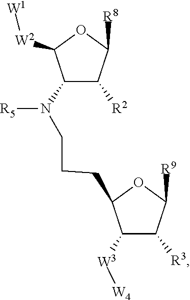

wherein W¹, W², W³, W⁴, R², R³, R⁸, and R⁹ are as defined herein. In certain embodiments, a compound of Formula (I′) is of the formula:

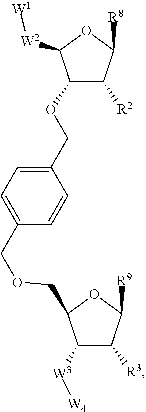

wherein W¹, W², W³, W⁴, R², R³, R⁸, and R⁹ are as defined herein. In certain embodiments, a compound of Formula (I′) is of the formula:

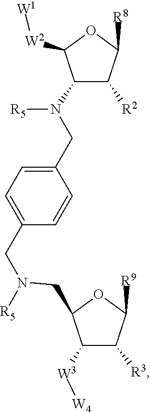

wherein W¹, W², W³, W⁴, R², R³, R⁸, and R⁹ are as defined herein. In certain embodiments, a compound of Formula (I′) is of the formula:

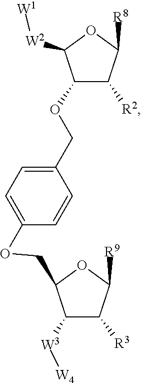

wherein W¹, W², W³, W⁴, R², R³, R⁸, and R⁹ are as defined herein. In certain embodiments, a compound of Formula (I′) is of the formula:

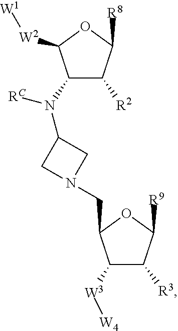

wherein W¹, W², W³, W⁴, R², R³, R⁸, and R⁹ are as defined herein. In certain embodiments, a compound of Formula (I′) is of the formula:

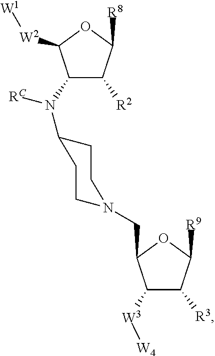

wherein W¹, W², W³, W⁴, R², R³, R⁸, and R⁹ are as defined herein. In certain embodiments, a compound of Formula (I′) is of the formula:

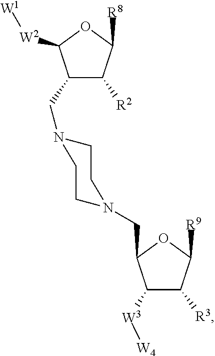

wherein W¹, W², W³, W⁴, R², R³, R⁸, and R⁹ are as defined herein. In certain embodiments, a compound of Formula (I′) is of the formula:

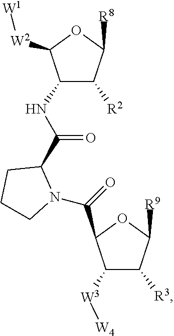

wherein W¹, W², W³, W⁴, R², R³, R⁸, and R⁹ are as defined herein. In certain embodiments, a compound of Formula (I′) is of the formula:

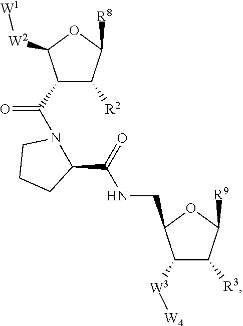

wherein W¹, W², W³, W⁴, R², R³, R⁸, and R⁹ are as defined herein. In certain embodiments, a compound of Formula (I′) is of the formula:

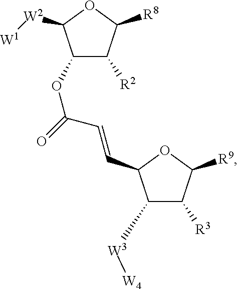

wherein W¹, W², W³, W⁴, R², R³, R⁸, and R⁹ are as defined herein. In certain embodiments, a compound of Formula (I′) is of the formula:

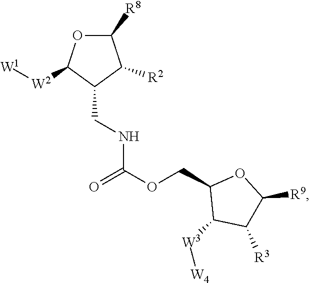

wherein W¹, W², W³, W⁴, R², R³, R⁸, and R⁹ are as defined herein. In certain embodiments, a compound of Formula (I′) is of the formula:

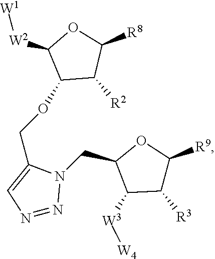

wherein W¹, W², W³, W⁴, R², R³, R⁸, and R⁹ are as defined herein. In certain embodiments, a compound of Formula (I′) is of the formula:

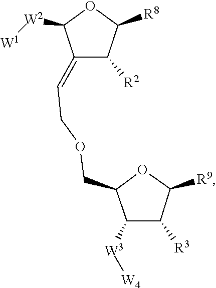

wherein W¹, W², W³, W⁴, R², R³, R⁸, and R⁹ are as defined herein. In certain embodiments, a compound of Formula (I′) is of the formula:

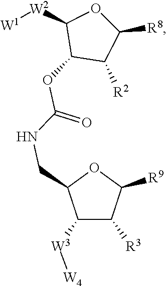

wherein W¹, W², W³, W⁴, R², R³, R⁸, and R⁹ are as defined herein. In certain embodiments, a compound of Formula (I′) is of the formula:

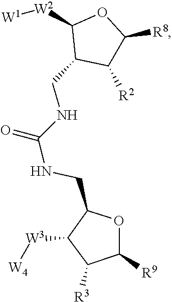

wherein W¹, W², W³, W⁴, R², R³, R⁸, and R⁹ are as defined herein. In certain embodiments, a compound of Formula (I′) is of the formula:

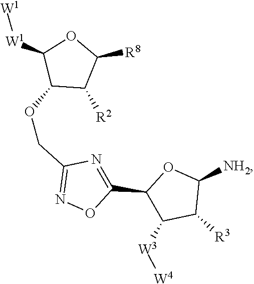

wherein W¹, W², W³, W⁴, R², R³, R⁸, and R⁹ are as defined herein. In certain embodiments, a compound of Formula (I′) is of the formula:

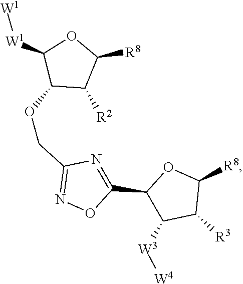

wherein W¹, W², W³, W⁴, R², R³, R⁸, and R⁹ are as defined herein. In certain embodiments, a compound of Formula (I′) is of the formula:

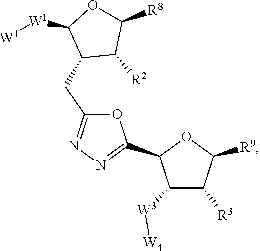

wherein W¹, W², W³, W⁴, R², R³, R⁸, and R⁹ are as defined herein. In certain embodiments, a compound of Formula (I′) is of the formula: wherein

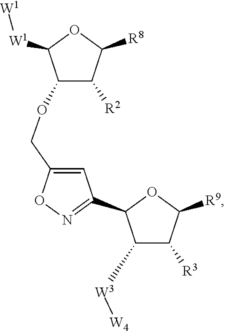

W¹, W², W³, W⁴, R², R³, R⁸, and R⁹ are as defined herein. In certain embodiments, a compound of Formula (I′) is of the formula:

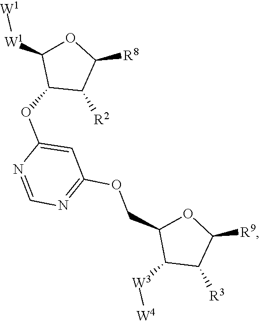

wherein W¹, W², W³, W⁴, R², R³, R⁸, and R⁹ are as defined herein. In certain embodiments, a compound of Formula (I′) is of the formula:

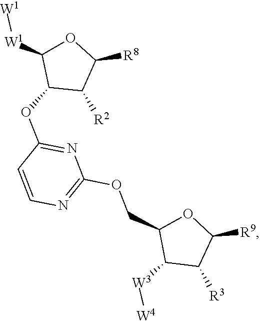

wherein W¹, W², W³, W⁴, R², R³, R⁸, and R⁹ are as defined herein.

In certain embodiments, the compound of Formula (I), or salt or prodrug thereof, is of the formula:

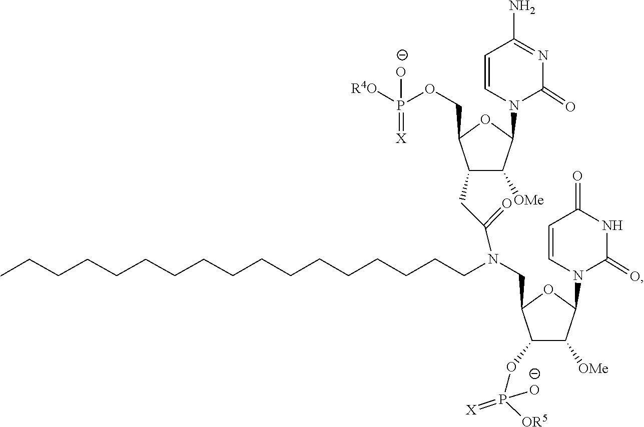

wherein:
R⁴ and R⁵ are independently an oligonucleotide or a protecting group; and
   each X is independently O or S.

In certain embodiments, the compound of Formula (I) is of the formula:

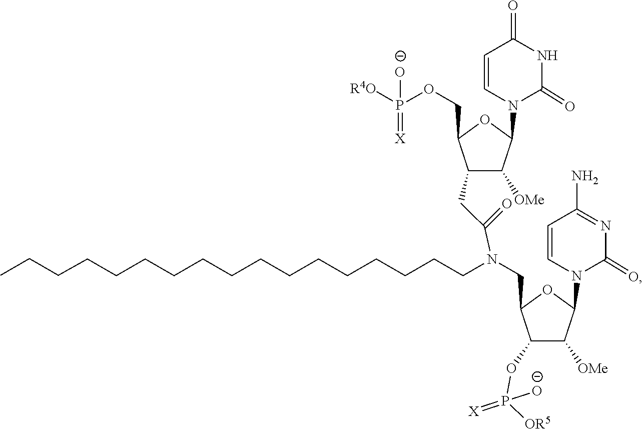

wherein:
R⁴ and R⁵ are independently an oligonucleotide or a protecting group; and
each X is independently O or S;
   or salt or prodrug thereof.

In certain embodiments, the compound of Formula (I) is of the formula:

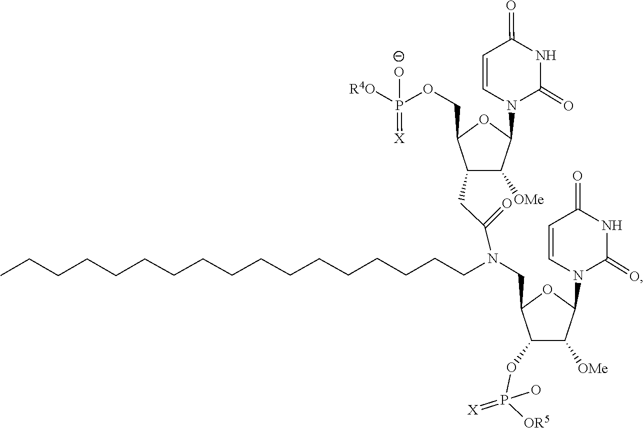

wherein:
R⁴ and R⁵ are independently an oligonucleotide or a protecting group; and
each X is independently O or S;
   or salt or prodrug thereof.

In certain embodiments, the compounds described herein contain the substituent W¹. In certain embodiments, W¹ is a modified or unmodified nucleoside. In certain embodiments, W¹ is an oligonucleotide. In certain embodiments, W¹ is a ligand. In certain embodiments, W¹ is a lipid. In certain embodiments, W¹ is a protecting group.

In certain embodiments, the compounds described herein contain the substituent W². In certain embodiments, W² is a bond. In certain embodiments, W² is a linker. In certain embodiments, W² is a substituted or unsubstituted alkylene. In certain embodiments, W² is a substituted or unsubstituted heteroalkylene. In certain embodiments, W² is a substituted or unsubstituted carbocyclylene. In certain embodiments, W² is a substituted or unsubstituted heterocyclylene. In certain embodiments, W² is a substituted or unsubstituted arylene. In certain embodiments, W² is a substituted or unsubstituted heteroarylene. In certain embodiments, W² is —O—. In certain embodiments, W² is —OP(O)O₂—. In certain embodiments, W² is —N(R^{A})—. In certain embodiments, W² is —S—. In certain embodiments, W² is —C(═O)—. In certain embodiments, W² is —C(═O)O—. In certain embodiments, W² is —C(═O)NR^{A}—. In certain embodiments, W² is —NR^{A}C(═O)—. In certain embodiments, W² is —NR^{A}C(═O)R^{A}—. In certain embodiments, W² is —C(═O)R^{A}—. In certain embodiments, W² is —NR^{A}C(═O)O—. In certain embodiments, W² is —NR^{A}C(═O)N(R^{A})—. In certain embodiments, W² is —OC(═O)—. In certain embodiments, W² is —OC(═O)O—. In certain embodiments, W² is —OC(═O)N(R^{A})—. In certain embodiments, W² is —S(O)₂NR^{A}—. In certain embodiments, W² is —NR^{A}SO₂—.

In certain embodiments, the compounds described herein contain the substituent W³. In certain embodiments, W³ is a bond. In certain embodiments, W³ is a linker. In certain embodiments, W³ is a substituted or unsubstituted alkylene. In certain embodiments, W³ is a substituted or unsubstituted heteroalkylene. In certain embodiments, W³ is a substituted or unsubstituted carbocyclylene. In certain embodiments, W³ is a substituted or unsubstituted heterocyclylene. In certain embodiments, W³ is a substituted or unsubstituted arylene. In certain embodiments, W³ is a substituted or unsubstituted heteroarylene. In certain embodiments, W³ is —O—. In certain embodiments, W³ is —OP(O)O₂—. In certain embodiments, W³ is —N(R^{A})—. In certain embodiments, W³ is —S—. In certain embodiments, W³ is —C(═O)—. In certain embodiments, W³ is —C(═O)O—. In certain embodiments, W³ is —C(═O)NR^{A}—. In certain embodiments, W³ is —NR^{A}C(═O)—. In certain embodiments, W³ is —NR^{A}C(═O)R^{A}—. In certain embodiments, W³ is —C(═O)R^{A}—. In certain embodiments, W³ is —NR^{A}C(═O)O—. In certain embodiments, W³ is —NR^{A}C(═O)N(R^{A})—. In certain embodiments, W³ is —OC(═O)—. In certain embodiments, W³ is —OC(═O)O—. In certain embodiments, W³ is —OC(═O)N(R^{A})—. In certain embodiments, W³ is —S(O)₂NR^{A}—. In certain embodiments, W³ is —NR^{A}SO₂—.

In certain embodiments, the compounds described herein contain the substituent W⁴. In certain embodiments, W⁴ is an modified or unmodified nucleoside. In certain embodiments, W⁴ is an oligonucleotide. In certain embodiments, W⁴ is a ligand. In certain embodiments, W⁴ is a lipid. In certain embodiments, W⁴ is a protecting group.

In certain embodiments, W¹ is a modified or unmodified nucleoside, and W⁴ is a modified or unmodified nucleoside. In certain embodiments, W¹ is a modified or unmodified nucleoside, and W⁴ is an oligonucleotide.

In certain embodiments, W¹ is a modified or unmodified nucleoside, W² is a bond, and W⁴ is a modified or unmodified nucleoside. In certain embodiments, W¹ is a modified or unmodified nucleoside, W² is a bond and W⁴ is an oligonucleotide.

In certain embodiments, W¹ is a modified or unmodified nucleoside, W² is a linker, and W⁴ is a modified or unmodified nucleoside. In certain embodiments, W¹ is a modified or unmodified nucleoside, W² is a linker and W⁴ is an oligonucleotide.

In certain embodiments, W¹ is a modified or unmodified nucleoside, W³ is a bond, and W⁴ is a modified or unmodified nucleoside. In certain embodiments, W¹ is a modified or unmodified nucleoside, W³ is a bond, and W⁴ is an oligonucleotide.

In certain embodiments, W¹ is a modified or unmodified nucleoside, W³ is a linker, and W⁴ is a modified or unmodified nucleoside. In certain embodiments, W¹ is a modified or unmodified nucleoside, W³ is a linker, and W⁴ is an oligonucleotide.

In certain embodiments, W¹ is a modified or unmodified nucleoside, W³ is a substituted or unsubstituted heteroalkylene, and W⁴ is a modified or unmodified nucleoside. In certain embodiments, W¹ is a modified or unmodified nucleoside, W³ is a substituted or unsubstituted heteroalkylene, and W⁴ is an oligonucleotide.

In certain embodiments, the compounds described herein contain the substituent Q¹. In certain embodiments, Q¹ is —H. In certain embodiments, Q¹ is —OR⁴. In certain embodiments, Q¹ is a ligand. In certain embodiments, Q¹ is a linker. In certain embodiments, Q¹ is a lipid.

In certain embodiments, the compounds as described herein contain the substituent Q². In certain embodiments, Q² is independently a bond. In certain embodiments, Q² is independently

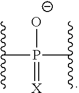

In certain embodiments, Q² is independently a ligand. In certain embodiments, Q² is independently a linker. In certain embodiments, Q² is independently a lipid.

In certain embodiments, the compounds described herein contain the substituent Q³. In certain embodiments, Q³ is independently a bond. In certain embodiments, Q³ is independently

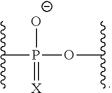

In certain embodiments, Q³ is independently a ligand. In certain embodiments, Q³ is independently a linker. In certain embodiments, Q³ is independently a lipid.

In certain embodiments, the compounds described herein contain the substituent Q⁴. In certain embodiments, Q⁴ is independently a bond, In certain embodiments, Q⁴ is independently —R¹⁰O—. In certain embodiments, Q⁴ is independently a ligand. In certain embodiments, Q⁴ is independently a linker. In certain embodiments, Q⁴ is independently a lipid.

In certain embodiments, the compounds described herein contains the substituent Q⁵. In certain embodiments, Q⁵ is independently a bond. In certain embodiments, Q⁵ is independently

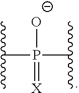

In certain embodiments, Q⁵ is independently a ligand. In certain embodiments, Q⁵ is independently a linker. In certain embodiments, Q⁵ is independently a lipid

In certain embodiments, the compounds described herein contain the substituent Q⁶. In certain embodiments, Q⁶ is independently a bond. In certain embodiments, Q⁶ is independently

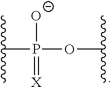

In certain embodiments, Q⁶ is independently a ligand. In certain embodiments, Q⁶ is independently a linker. In certain embodiments, Q⁶ is independently a lipid.

In certain embodiments, the compounds described herein contain the substituent Q⁷. In certain embodiments, Q⁷ is independently —H. In certain embodiments, Q⁷ is independently —R⁵. In certain embodiments, Q⁷ is independently a ligand. In certain embodiments, Q⁷ is independently a linker. In certain embodiments, Q⁷ is independently a lipid.

In certain embodiments, the compounds described herein contain the substituent Y. In certain embodiments, Y is independently substituted or unsubstituted alkylene. In certain embodiments, Y is independently substituted or unsubstituted heteroalkylene. In certain embodiments, Y is independently substituted or unsubstituted carbocyclylene. In certain embodiments, Y is independently substituted or unsubstituted heterocyclylene. In certain embodiments, Y is independently substituted or unsubstituted arylene. In certain embodiments, Y is independently substituted or unsubstituted heteroarylene. In certain embodiments, Y is independently —O—. In certain embodiments, Y is independently —OP(O)O₂—. In certain embodiments, Y is independently —N(R^{C})—. In certain embodiments, Y is independently —S—. In certain embodiments, Y is independently —C(═O)—. In certain embodiments, Y is independently —C(═O)O—. In certain embodiments, Y is independently C(═O)N(RC)—. In certain embodiments, Y is independently N(R^{C})C(═O)—. In certain embodiments, Y is independently —NR^{C}C(═O)R^{C}—. In certain embodiments, Y is independently —C(═O)R^{C}—. In certain embodiments, Y is independently —NR^{C}C(═O)O—. In certain embodiments, Y is independently —NR^{C}C(═O)N(R^{C})—. In certain embodiments, Y is independently —OC(═O)—. In certain embodiments, Y is independently —OC(═O)O—. In certain embodiments, Y is independently —OC(═O)N(R^{C})—. In certain embodiments, Y is independently —S(O)₂NR^{C}—. In certain embodiments, Y is independently —NR^{C}SO₂—.

In certain embodiments, R^{C} is independently —H. In certain embodiments, R^{C} is independently substituted or unsubstituted alkyl. In certain embodiments, R^{C} is independently substituted or unsubstituted alkenyl. In certain embodiments, R^{C} is independently substituted or unsubstituted alkynyl. In certain embodiments, R^{C} is independently substituted or unsubstituted heteroalkyl. In certain embodiments, R^{C} is independently substituted or unsubstituted aryl. In certain embodiments, R^{C} is independently substituted or unsubstituted heteroaryl. In certain embodiments, R^{C} is independently a substituted or unsubstituted lipophilic moiety. In certain embodiments, each instance of R^{C} is independently alkyl. In certain embodiments, each instance of R^{C} is independently —C₈-C₁₀₀-alkyl. In certain embodiments, each instance of R^{C} is independently —C₈-C₄₀-alkyl. In certain embodiments, each instance of R^{C} is independently —C₈-C₂₀-alkyl. In certain embodiments, each instance of R^{C} is independently —C₁₂-C₂₀-alkyl. In certain embodiments, each instance of R^{C} is independently —C₁₆-C₂₀-alkyl. In certain embodiments, each instance of R^{C} is independently —H.

In certain embodiments, R^{C} is a tocopherol (e.g., an α (alpha), β (beta), γ (gamma), or δ (delta) tocopherol). In certain embodiments, R^{C} is Vitamin E

In certain embodiments, R^{c} is a saturated or unsaturated C₁-C₃₀ hydrocarbon chain (e.g., C₁-C₃₀ alkyl or alkenyl) optionally substituted with a functional group selected from the group consisting of hydroxyl, amine, carboxylic acid, azide, and alkyne.

In some embodiments, R^{c} group contains a saturated or unsaturated C₁-C₁₇ hydrocarbon chain (e.g., a linear C₁-C₁₇ alkyl or alkenyl). In one embodiment, the lipophilic moiety contains a saturated or unsaturated C₁₇ hydrocarbon chain.

In some embodiments, R^{c} group is a C₁-C₃₀ acid (e.g., hexanoic acid, heptanoic acid, octanoic acid, nonanoic acid, decanoic acid, undecanoic acid, dodcanoic acid, tridecanoic acid, tetradecanoic acid, pentadecanoic acid, hexadecanoic acid, heptadecanoic acid, octadecanoic acid, oleic acid, linoleic acid, arachidonic acid, anandamide.

In certain embodiments, the compounds described herein contain the substituent R². In certain embodiments, R² is independently —H. In certain embodiments, R² is independently —OR⁶. In certain embodiments, R² is independently a halogen. In certain embodiments, R² is independently —F. In certain embodiments, R² is independently substituted or unsubstituted alkyl. In certain embodiments, R² is independently substituted or unsubstituted alkenyl. In certain embodiments, R² is independently substituted or unsubstituted alkynyl. In certain embodiments, R² is independently —OMe. In certain embodiments, R² is independently —N(R⁶). In certain embodiments, R² is independently —SR⁶.

In certain embodiments, the compounds described herein contain the substituent R³. In certain embodiments, R³ is independently —H. In certain embodiments, R³ is independently —OR⁷. In certain embodiments, R³ is independently a halogen. In certain embodiments, R³ is independently —F. In certain embodiments, R³ is independently substituted or unsubstituted alkyl. In certain embodiments, R³ is independently substituted or unsubstituted alkenyl. In certain embodiments, R³ is independently substituted or unsubstituted alkynyl. In certain embodiments, R³ is independently —OMe. In certain embodiments, R³ is independently —N(R⁷). In certain embodiments, R³ is independently —SR⁷.

In certain embodiments, the compounds described herein contain the substituent R⁴. In certain embodiments, R⁴ is an oligonucleotide. In certain embodiments, R⁴ is a protecting group.

In certain embodiments, the compounds described herein contain the substituent R⁵. In certain embodiments, R⁵ is an oligonucleotide. In certain embodiments, R⁵ is a protecting group.

In certain embodiments, R⁴ and R⁵ are each independently an oligonucleotide.

In certain embodiments, R⁴ is an oligonucleotide; and R⁵ is a protecting group.

In certain embodiments, R⁴ is a protecting group; and R⁵ is an oligonucleotide.

In certain embodiments, R⁴ and R⁵ are each independently a protecting group.

In some embodiments, R⁴ and R⁵ are joined together to form a single oligonucleotide.

In certain embodiments, the compounds described herein contain the substituent R⁶. In certain embodiments, R⁶ is independently substituted or unsubstituted alkyl. In certain embodiments, R⁶ is independently substituted or unsubstituted heteroalkyl.

In certain embodiments, the compounds described herein contain the substituent R⁷. In certain embodiments, R⁷ is independently substituted or unsubstituted alkyl. In certain embodiments, R⁷ is independently substituted or unsubstituted heteroalkyl.

In certain embodiments, the compounds described herein contain the substituent R⁸. In certain embodiments, R⁸ is independently uracil. In certain embodiments, R⁸ is independently cytosine. In certain embodiments, R⁸ is independently adenine. In certain embodiments, R⁸ is independently guanine. In certain embodiments, R⁸ is independently inosine. In certain embodiments, R⁸ is independently thymine. In certain embodiments, R⁸ is independently substituted or unsubstituted heteroaryl. In certain embodiments, R⁸ is independently a nucleobase. In certain embodiments, R⁸ is independently a modified nucleobase.

In certain embodiments, the compounds described herein contain the substituent R⁹. In certain embodiments, R⁹ is independently uracil. In certain embodiments, R⁹ is independently cytosine. In certain embodiments, R⁹ is independently adenine. In certain embodiments, R⁹ is independently guanine. In certain embodiments, R⁹ is independently inosine. In certain embodiments, R⁹ is independently thymine. In certain embodiments, R⁹ is independently substituted or unsubstituted heteroaryl. In certain embodiments, R⁹ is independently a nucleobase. In certain embodiments, R⁹ is independently a modified nucleobase.

In certain embodiments, the compounds described herein contain the substituent R¹⁰. In certain embodiments, R¹⁰ is independently an oligonucleotide.

In certain embodiments, the compounds described herein contain the substituent X. In certain embodiments, X is independently O. In certain embodiments, X is independently S.

In certain embodiments, the compounds described herein contain the substituent Z¹, Z², Z³, or Z⁴. In certain embodiments, Z¹, Z², Z³, or Z⁴ is independently a bond. In certain embodiments, Z¹, Z², Z³, or Z⁴ is independently, C₁-C₆ alkylene (e.g., methylene, ethylene, propylene). In certain embodiments, Z¹, Z², Z³, or Z⁴ is independently C₂-C₆ alkenylene. In certain embodiments, Z¹, Z², Z³, or Z⁴ is independently

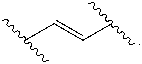 [SMILES: */C=C/*]

In certain embodiments, Z¹, Z², Z³, or Z⁴ is independently

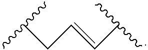 [SMILES: */C=C/C*]

In certain embodiments, the compounds as described herein contain the variable p. In certain embodiments, p is 0. In certain embodiments, p is 1. In certain embodiments, p is 0 or 1. In certain embodiments, p is 1, 2, or 3. In certain embodiments, p is 1. In certain embodiments, p is 2. In certain embodiments, p is 3. In certain embodiments, p is 4. In certain embodiments, p is 5. In certain embodiments, p is 6. In certain embodiments, p is 7. In certain embodiments, p is 8. In certain embodiments, p is 9. In certain embodiments, p is 10.

In certain embodiments, the compounds as described herein contain the variable n. In certain embodiments, n is 0. In certain embodiments, n is 1. In certain embodiments, n is 0 or 1. In certain embodiments, n is 1, 2, or 3. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10.

In certain embodiments, the compounds as described herein contain the variable m. In certain embodiments, m is 0. In certain embodiments, m is 1. In certain embodiments, m is 0 or 1. In certain embodiments, m is 1, 2, or 3. In certain embodiments, m is 1. In certain embodiments, m is 2. In certain embodiments, m is 3. In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10.

In certain embodiments, provided herein is an oligonucleotide comprising at least one dinucleotide of the formula:

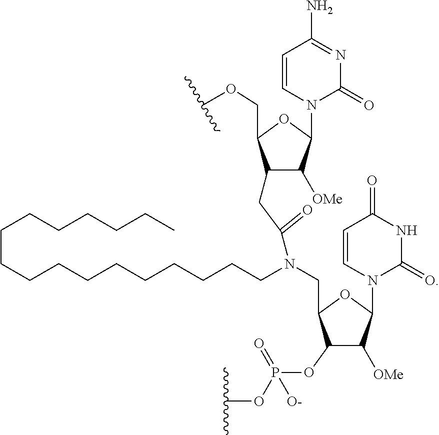

In certain embodiments, provided herein is an oligonucleotide comprising at least one dinucleotide of the formula:

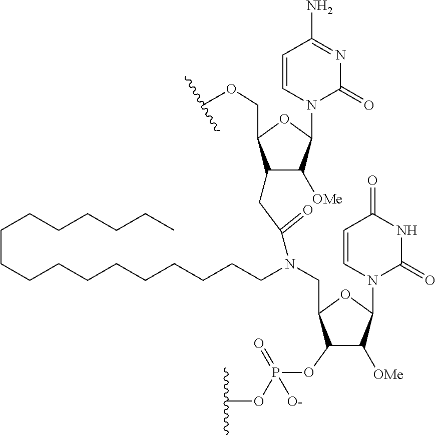

attached at the 3^{rd} and 4^{th} nucleoside from the 5′ end of the sense strand.

In certain embodiments, provided herein is an oligonucleotide comprising at least one dinucleotide of the formula:

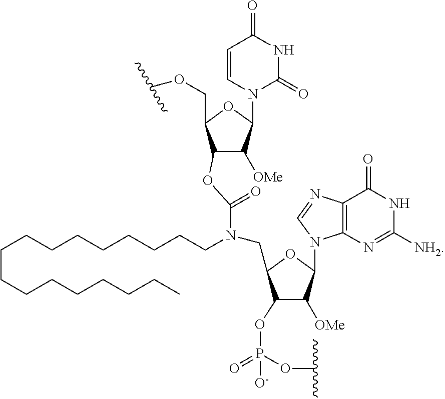

In certain embodiments, provided herein is an oligonucleotide comprising at least one dinucleotide of the formula:

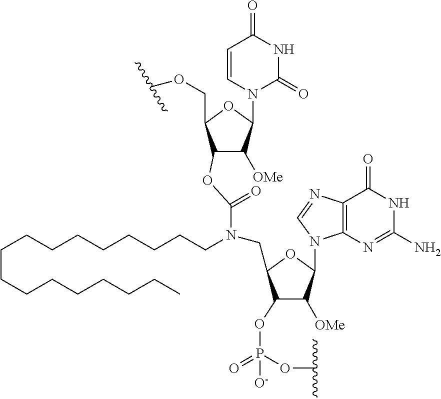

attached at the 4^{th} and 5^{th} nucleoside from the 5′ end on the sense strand.

In certain embodiments, provided herein is an oligonucleotide comprising at least one dinucleotide of the formula:

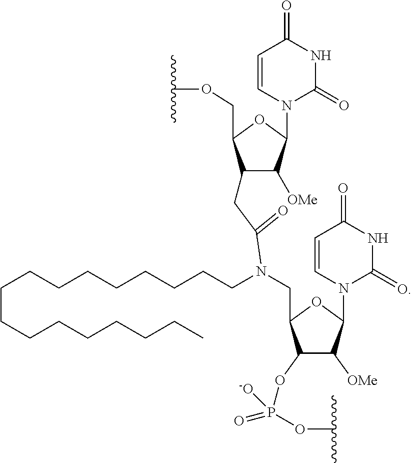

In certain embodiments, provided herein is an oligonucleotide comprising at least one dinucleotide of the formula:

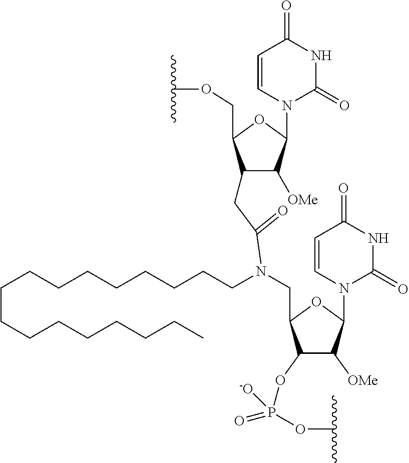

attached at the 15^{th} and 16^{th} nucleoside from the 5′end on the sense strand.

In certain embodiments, provided herein is an oligonucleotide comprising at least one dinucleotide of the formula:

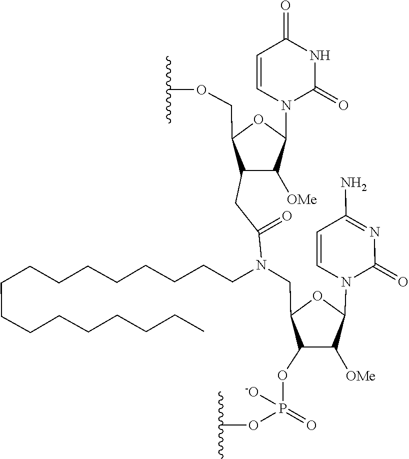

In certain embodiments, provided herein is an oligonucleotide comprising at least one dinucleotide of the formula:

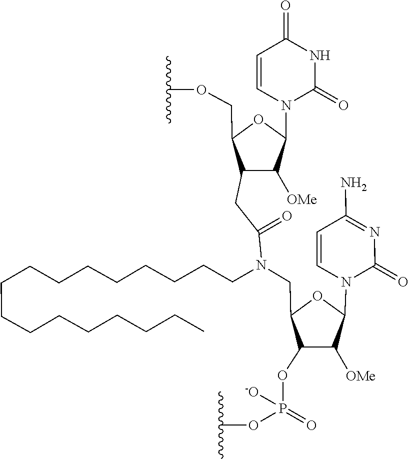

attached at the 16^{th} and 17^{th} nucleoside from the 5′ end on the sense strand.

In certain embodiments, provided herein is an oligonucleotide comprising at least one dinucleotide of the formula:

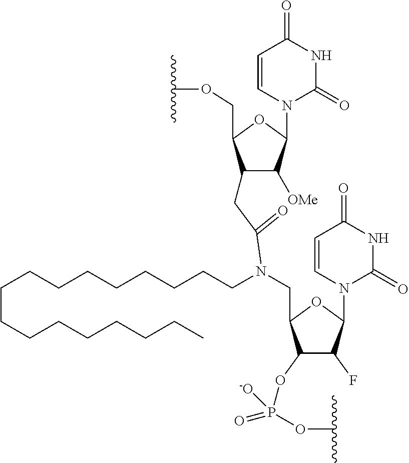

In certain embodiments, provided herein is an oligonucleotide comprising at least one dinucleotide of the formula:

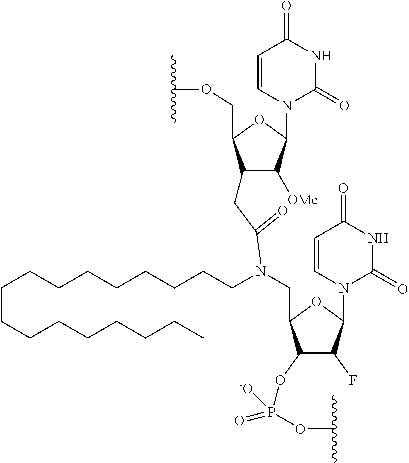

attached at the 6^{th} and 7^{th} nucleoside from the 5′end on the sense strand.

In certain embodiments, provided herein is an oligonucleotide comprising at least one dinucleotide of the formula:

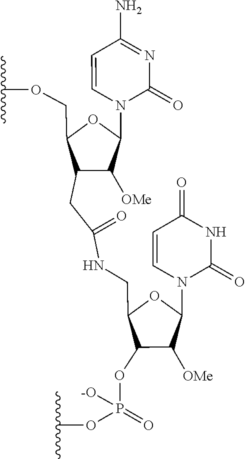

In certain embodiments, provided herein is an oligonucleotide comprising at least one dinucleotide of the formula:

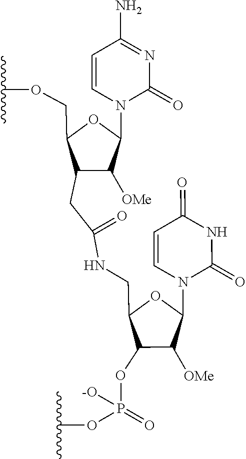

attached at the third and fourth nucleoside from the 5′ end of the sense strand.

In certain embodiments, provided herein is an oligonucleotide comprising at least one dinucleotide of the formula:

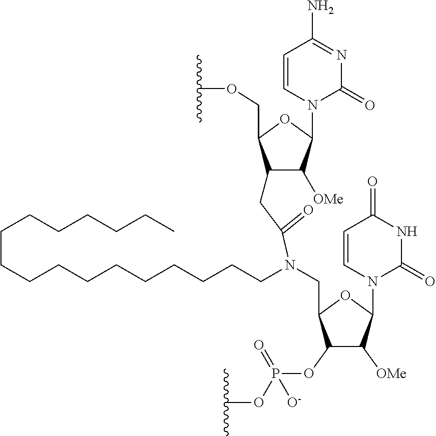

In certain embodiments, provided herein is an oligonucleotide comprising at least one dinucleotide of the formula:

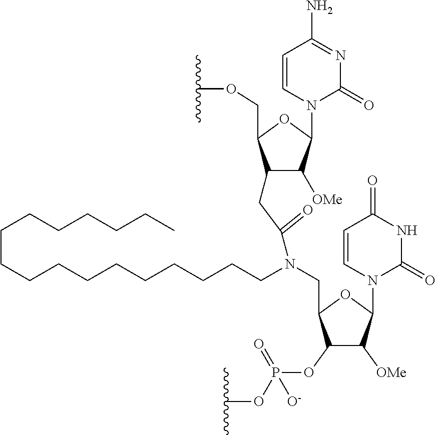

attached at the third and fourth nucleoside from the 5′ end of the sense strand.

In certain embodiments, provided herein is an oligonucleotide comprising at least one dinucleotide of the formula:

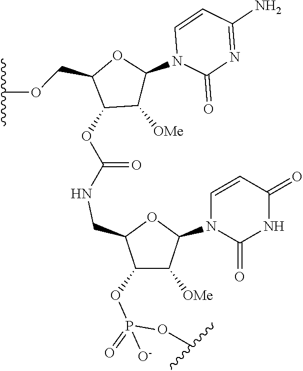 [SMILES: *OC[C@H]1O[C@@H](n2ccc(N)nc2=O)[C@H](OC)[C@@H]1OC(=O)NC[C@H]1O[C@@H](n2ccc(=O)[nH]c2=O)[C@H](OC)[C@@H]1OP(=O)([O-])O*]

In certain embodiments, provided herein is an oligonucleotide comprising at least one dinucleotide of the formula:

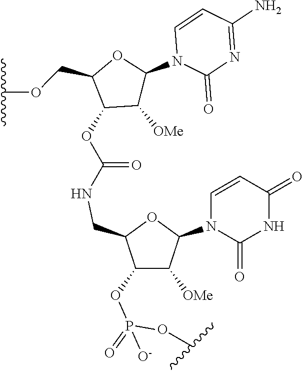 [SMILES: *OC[C@H]1O[C@@H](n2ccc(N)nc2=O)[C@H](OC)[C@@H]1OC(=O)NC[C@H]1O[C@@H](n2ccc(=O)[nH]c2=O)[C@H](OC)[C@@H]1OP(=O)([O-])O*]

attached at the third and fourth nucleoside from the 5′ end of the sense strand.

In certain embodiments, provided herein is an oligonucleotide comprising at least one dinucleotide of the formula:

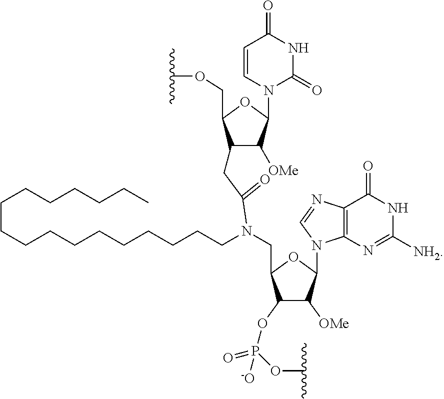

In certain embodiments, provided herein is an oligonucleotide comprising at least one dinucleotide of the formula:

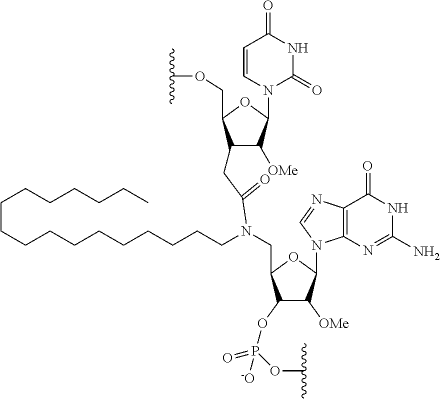

attached at the 4^{th} and 5th nucleoside from the 5′end on the sense strand.

In certain embodiments, provided herein is an oligonucleotide comprising at least one dinucleotide of the formula:

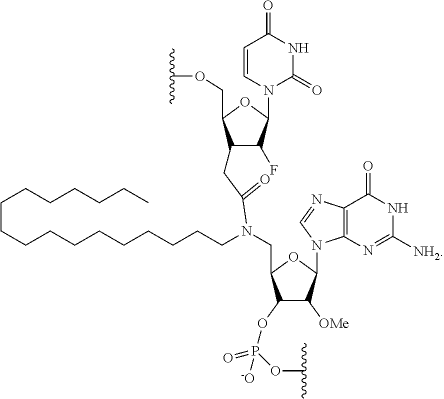

In certain embodiments, provided herein is an oligonucleotide comprising at least one dinucleotide of the formula:

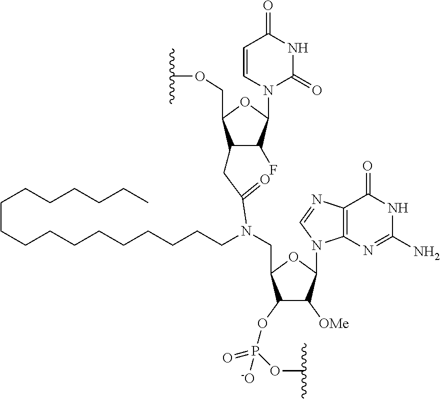

attached at the 7^{th} and 8th nucleoside from the 5′end on the sense strand.

In certain embodiments, provided herein is an oligonucleotide comprising at least one dinucleotide of the formula:

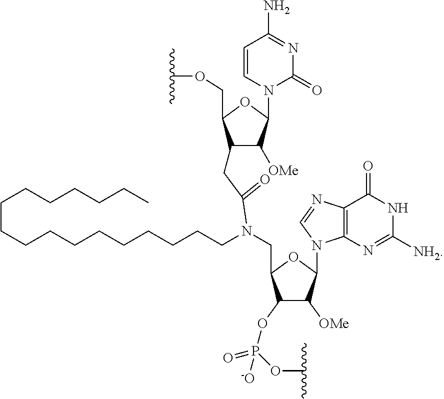

In certain embodiments, provided herein is an oligonucleotide comprising at least one dinucleotide of the formula:

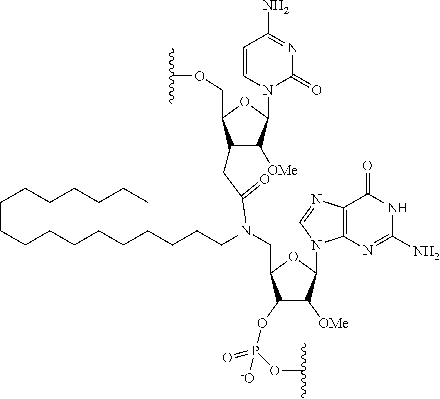

attached at the 17^{th} and 18th nucleoside from the 5′end on the sense strand.

In certain embodiments, provided herein is an oligonucleotide comprising at least one dinucleotide of the formula:

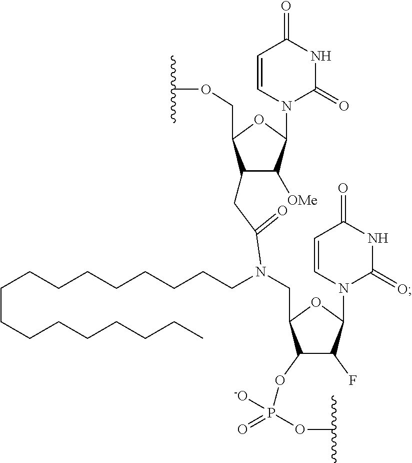

and further comprising at least one dinucleotide of the formula:

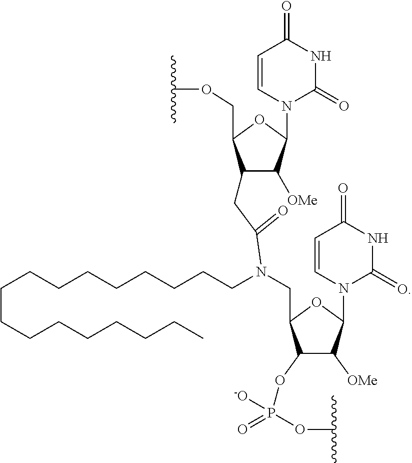

In certain embodiments, provided herein is an oligonucleotide comprising at least one dinucleotide of the formula:

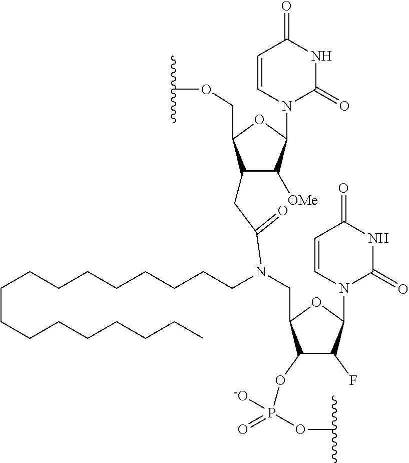

attached at the 6^{th} and 7^{th} nucleoside from the 5′end on the sense strand and the following structure:

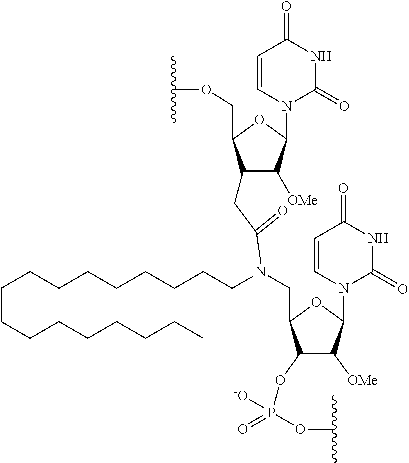

attached at the 15^{th} and 16^{th} nucleoside from the 5′ end on the sense strand.

In certain embodiments, provided herein is an oligonucleotide comprising at least one dinucleotide of the formula:

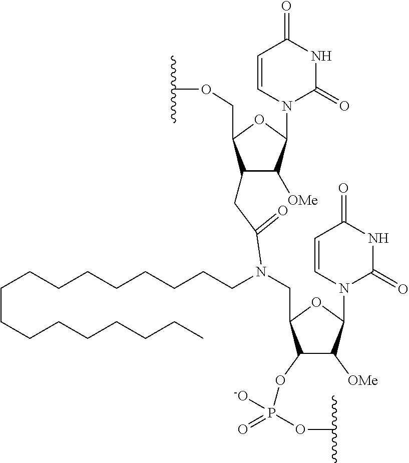

In certain embodiments, provided herein is an oligonucleotide comprising at least one dinucleotide of the formula:

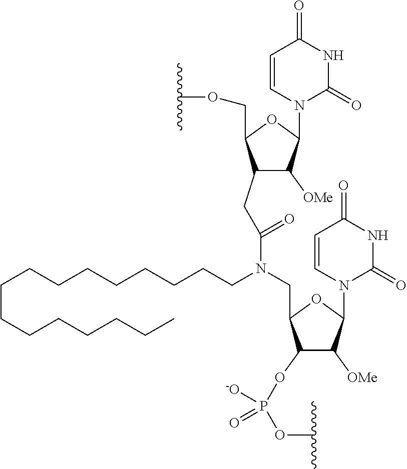

attached at the 5^{th} and 6^{th} nucleoside from the 5′ end on the sense strand.

In certain embodiments, provided herein is an oligonucleotide comprising at least one dinucleotide of the formula:

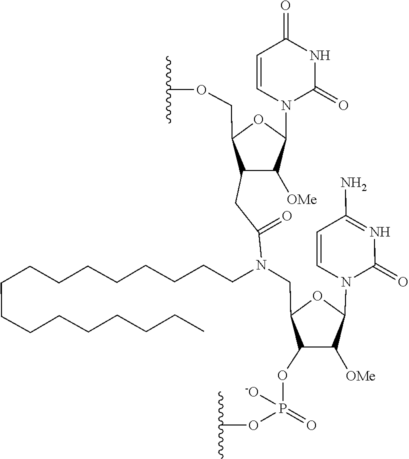

In certain embodiments, provided herein is an oligonucleotide comprising at least one dinucleotide of the formula:

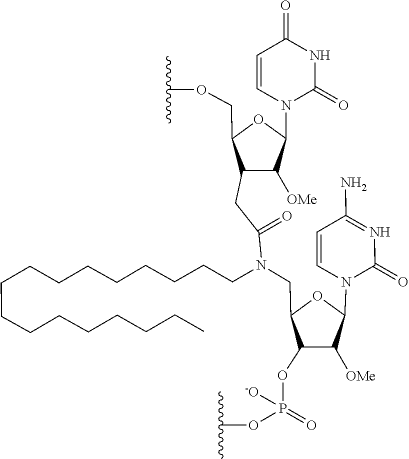

attached at the 12^{th} and 13^{th} nucleoside from the 5′ end on the sense strand

In certain embodiments, the present disclosure provides compounds of the formula:

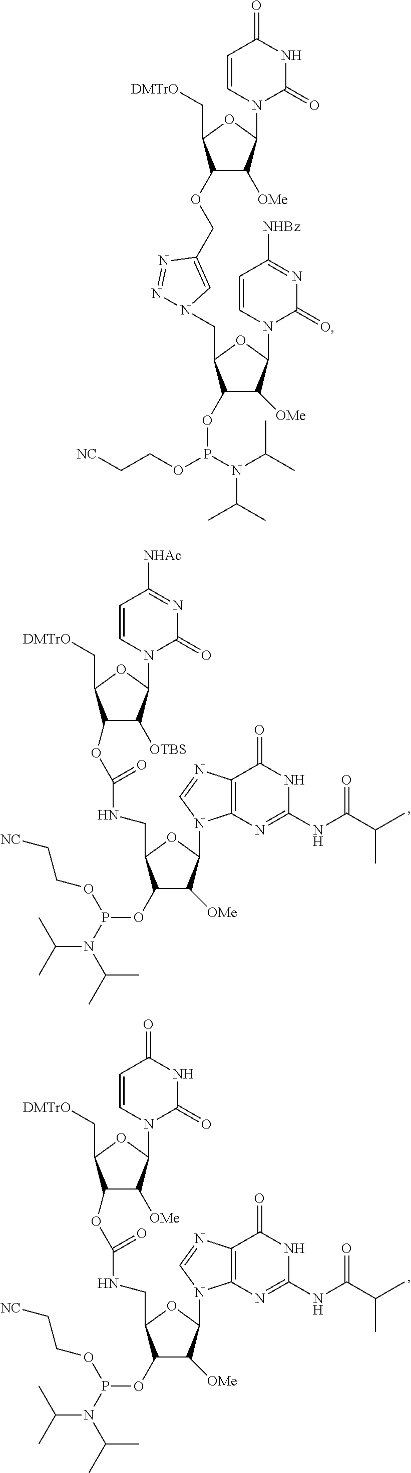

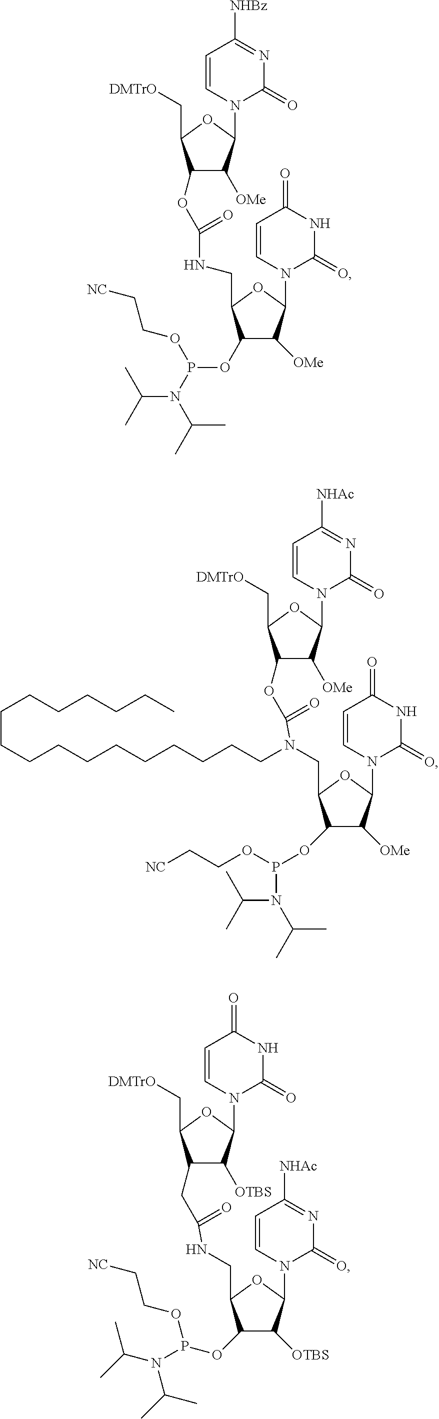

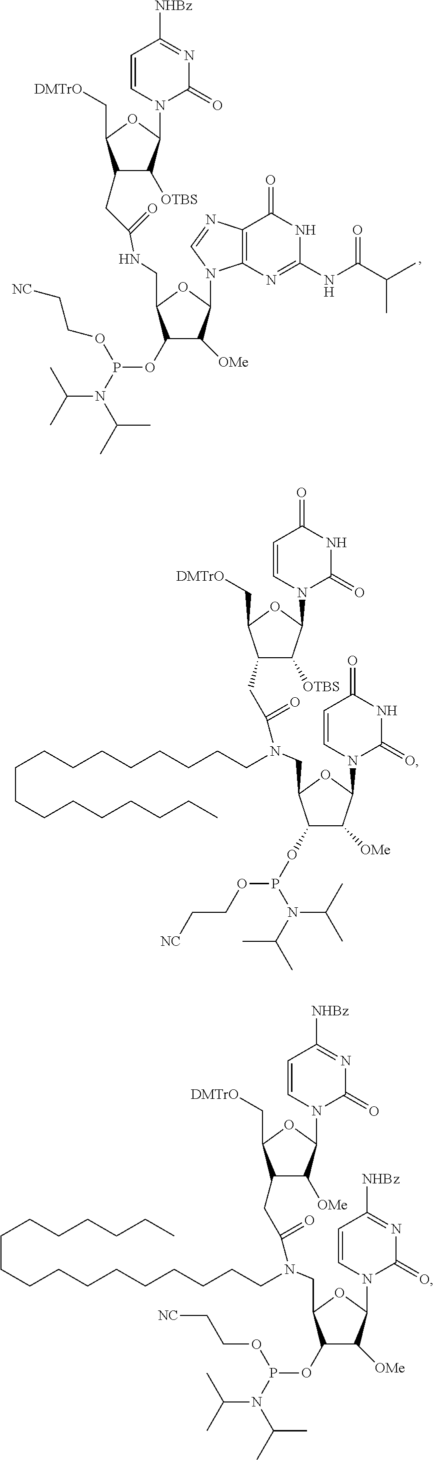

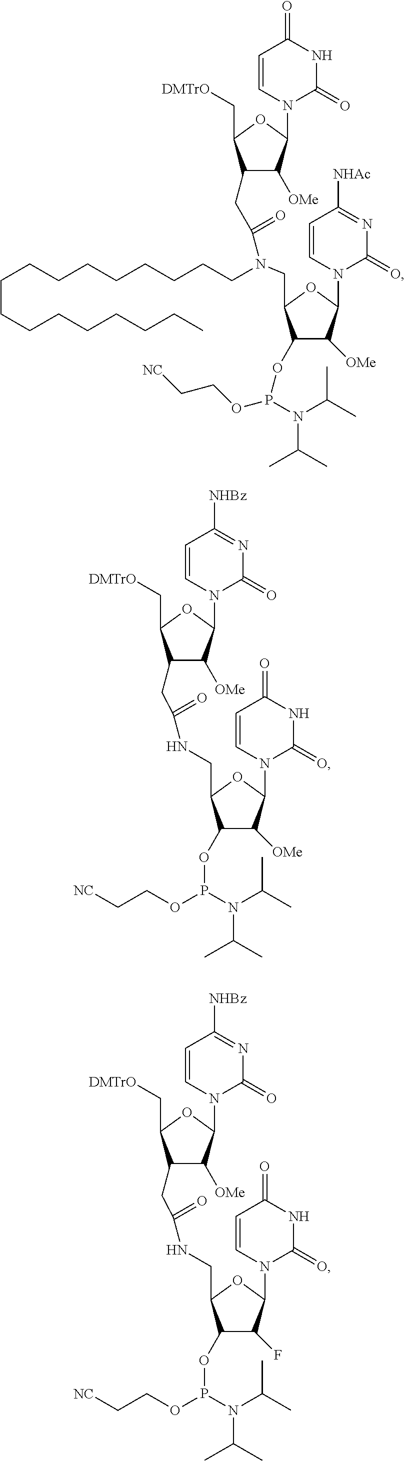

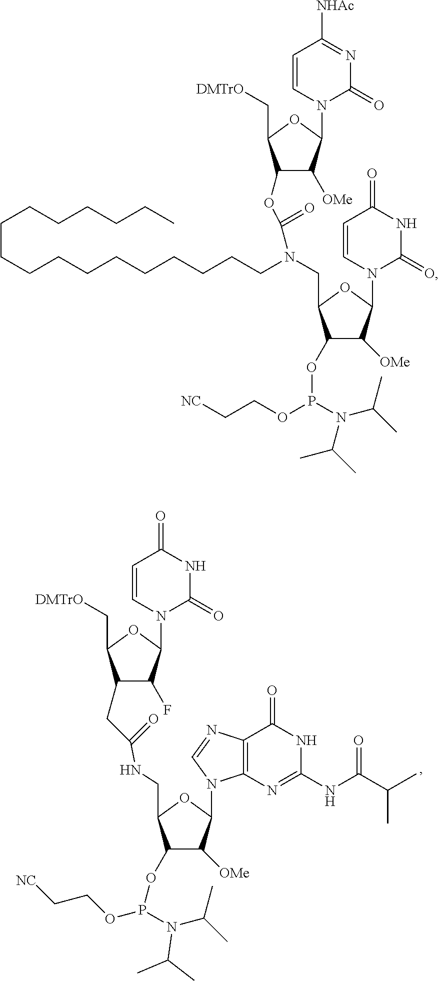

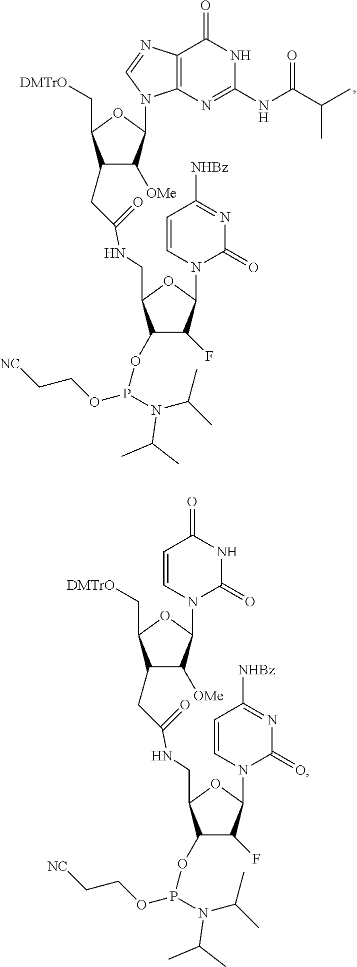

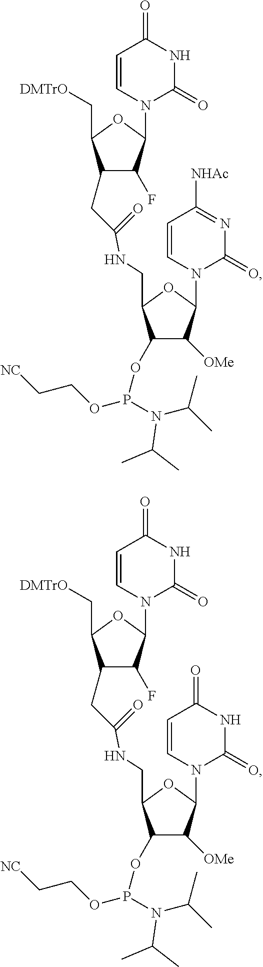

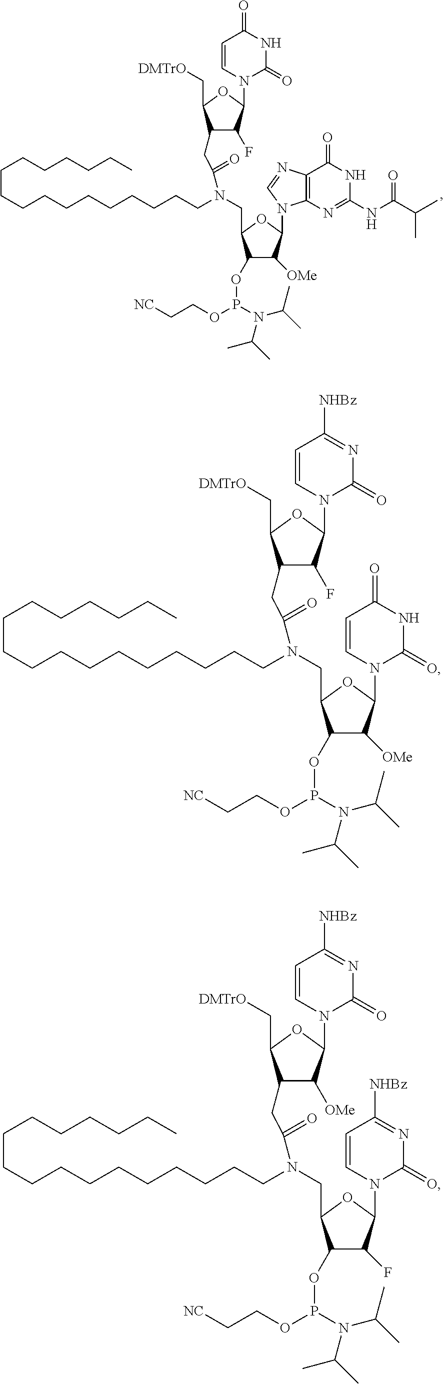

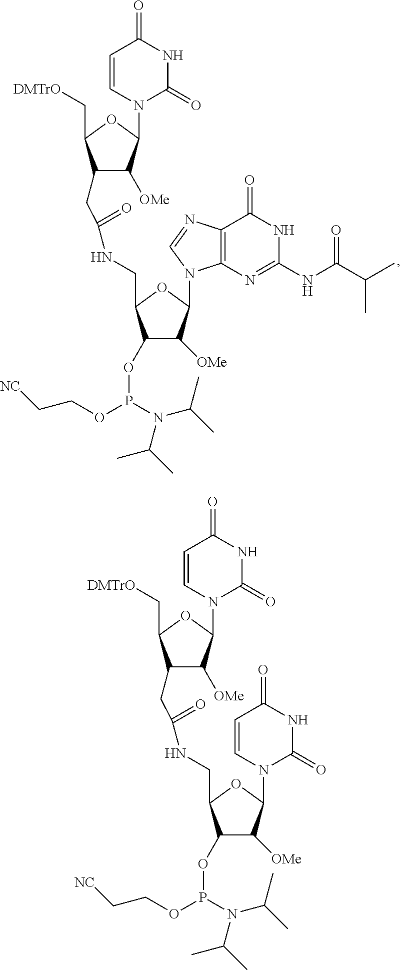

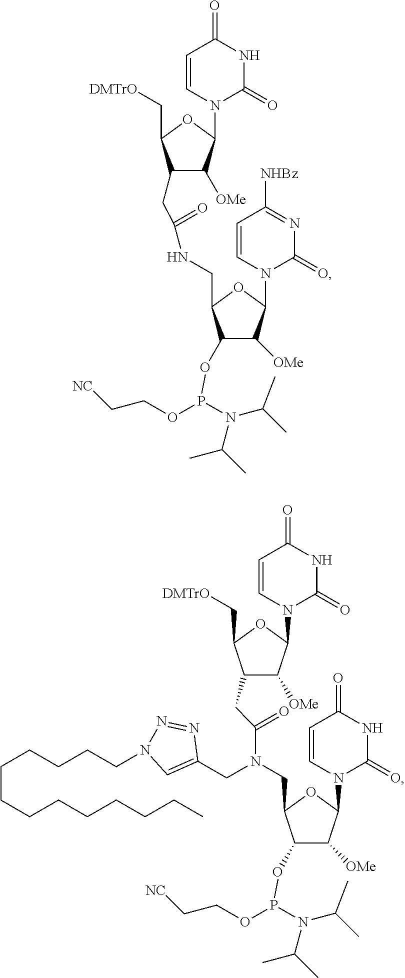

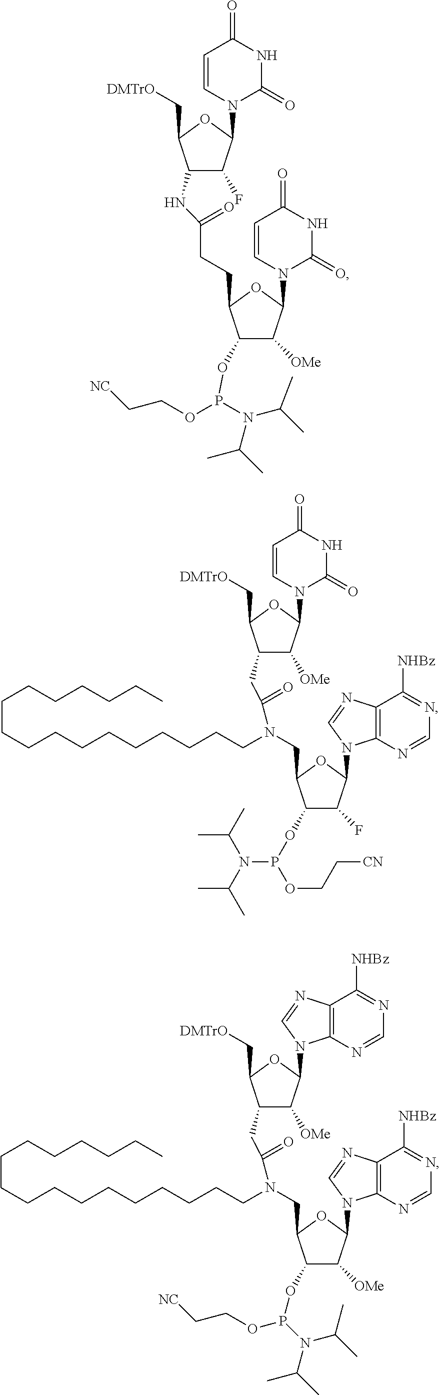

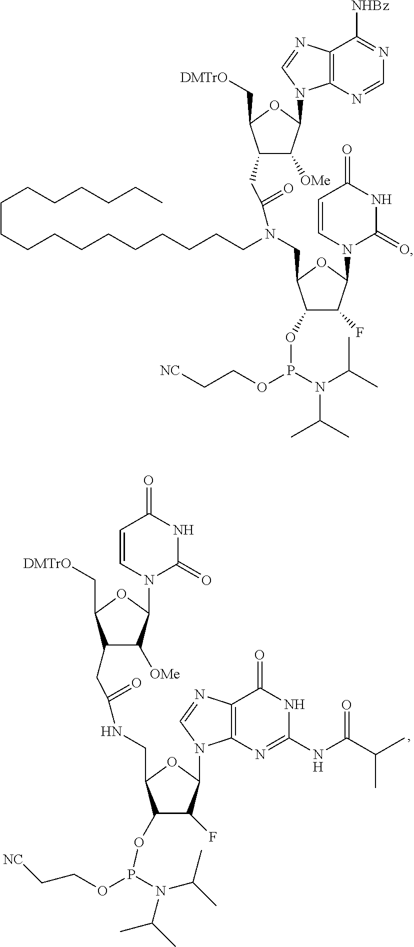

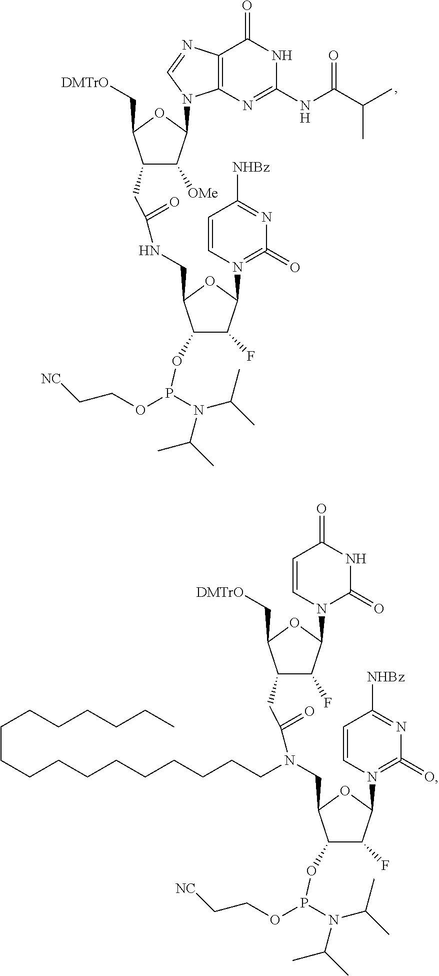

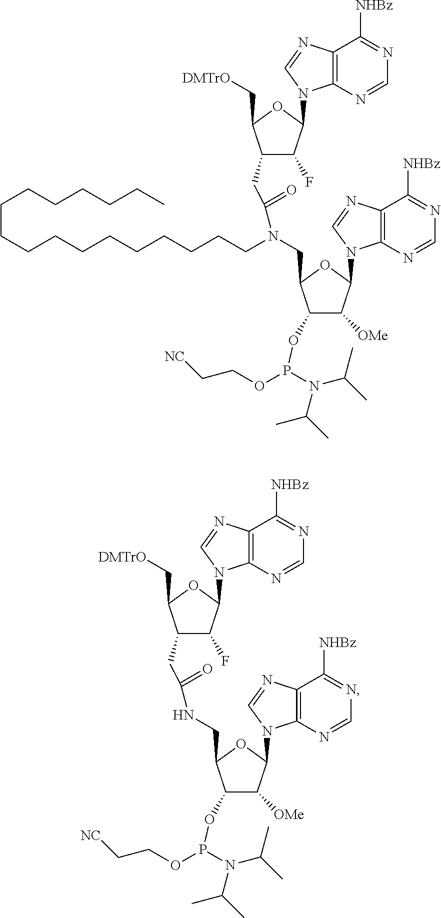

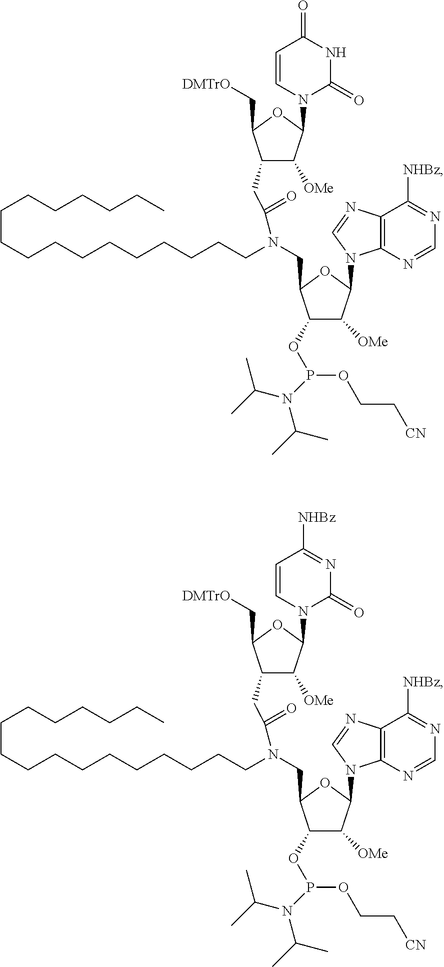

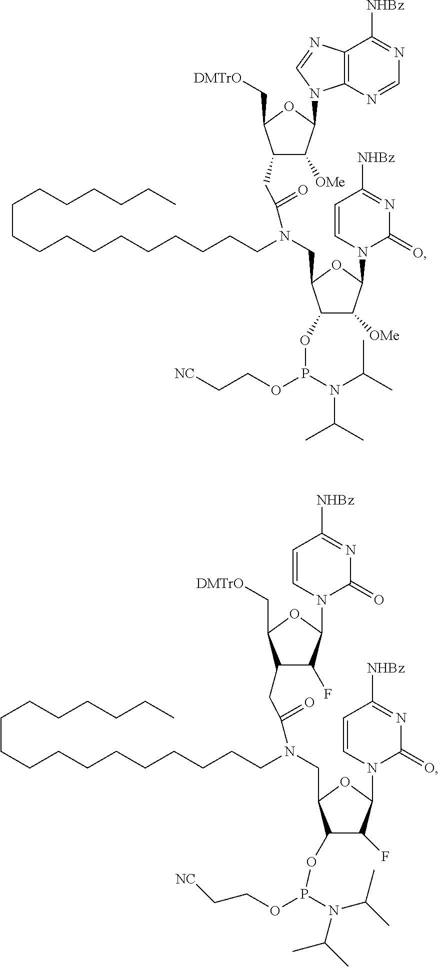

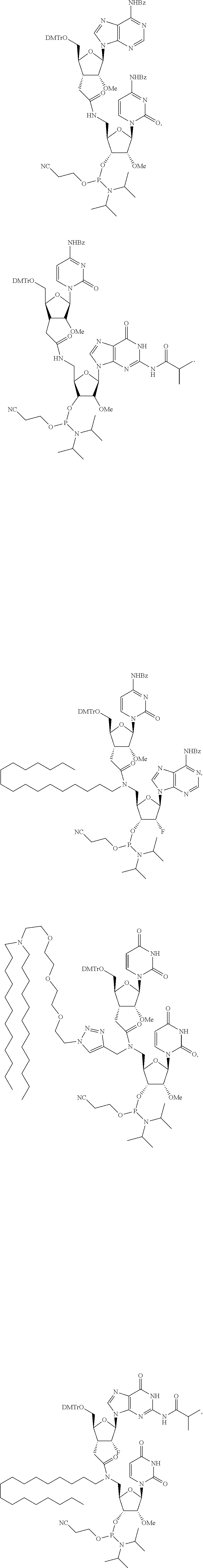

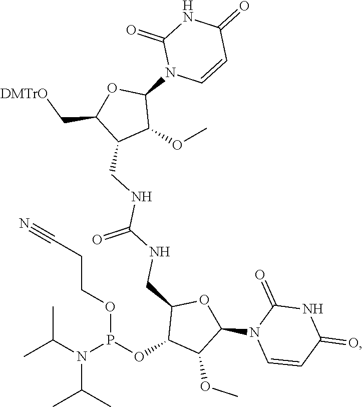

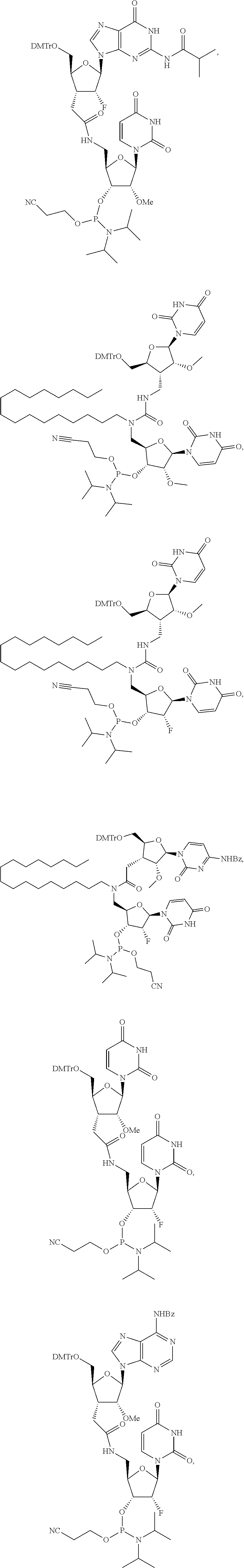

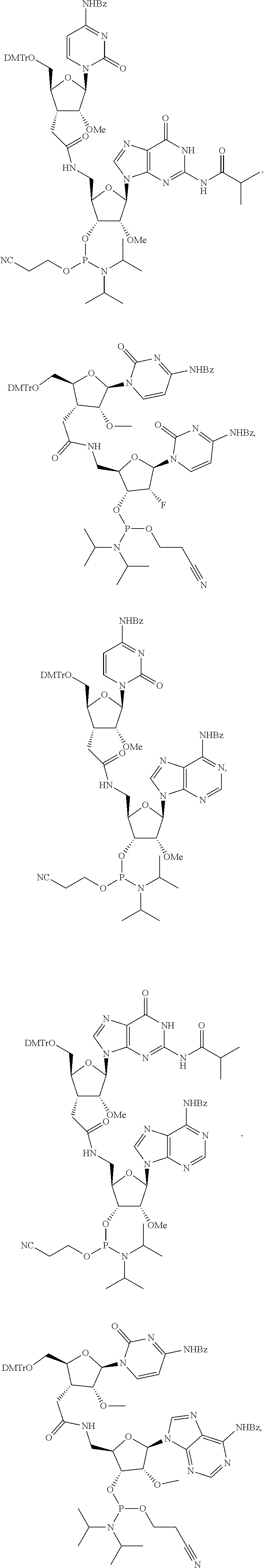

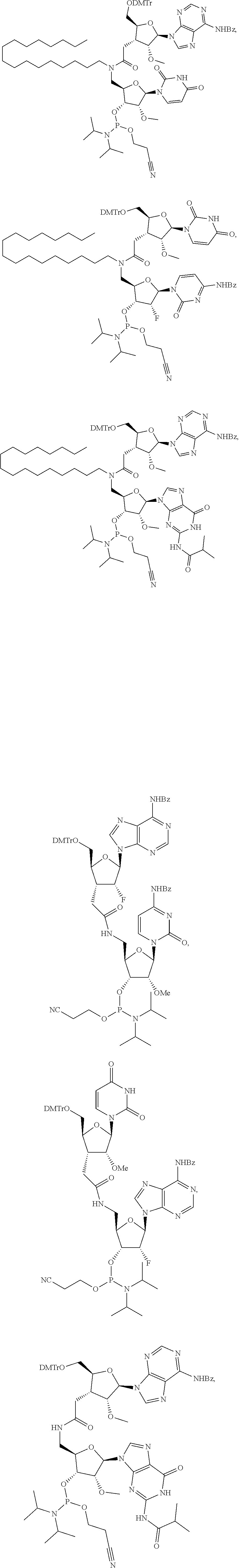

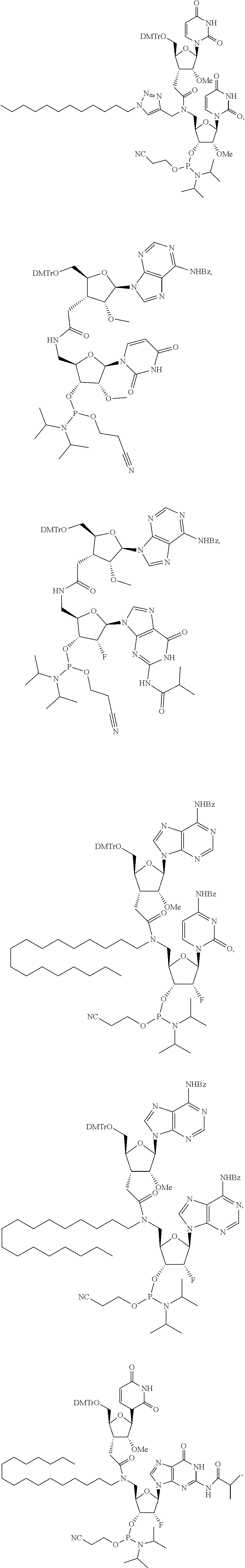

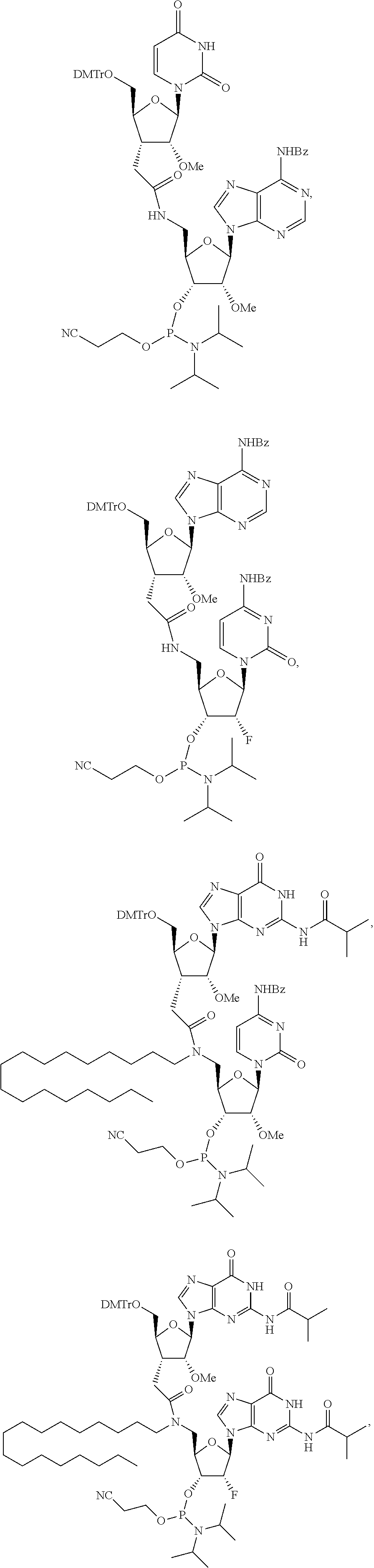

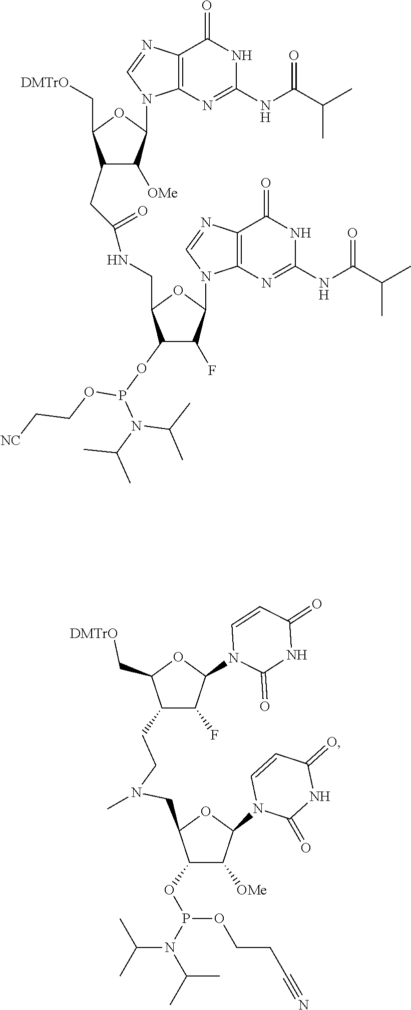

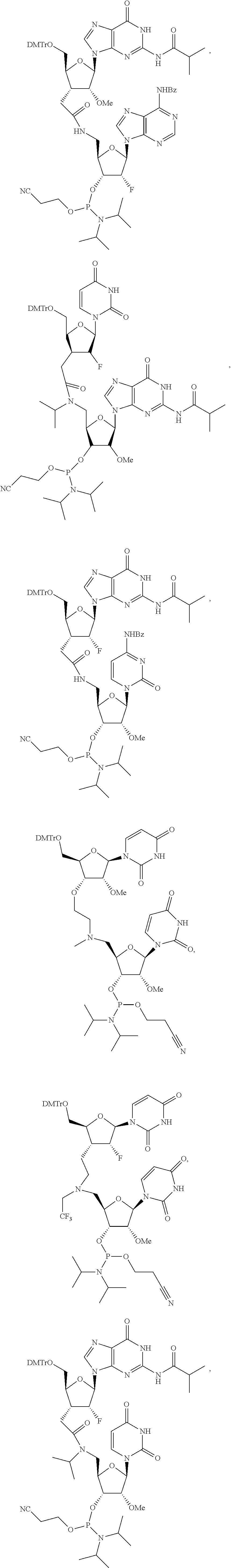

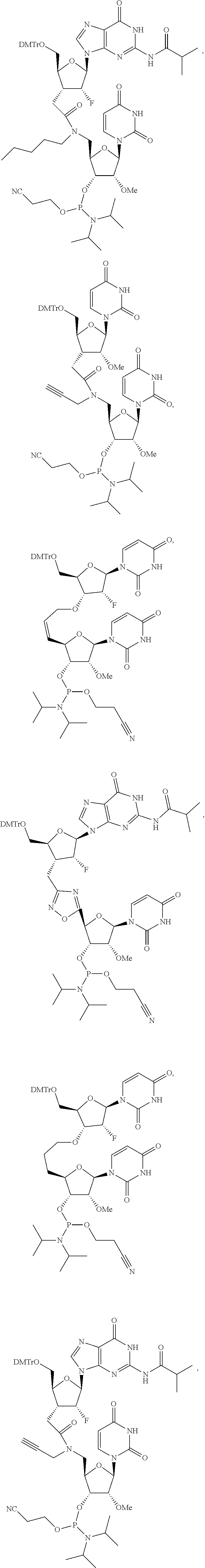

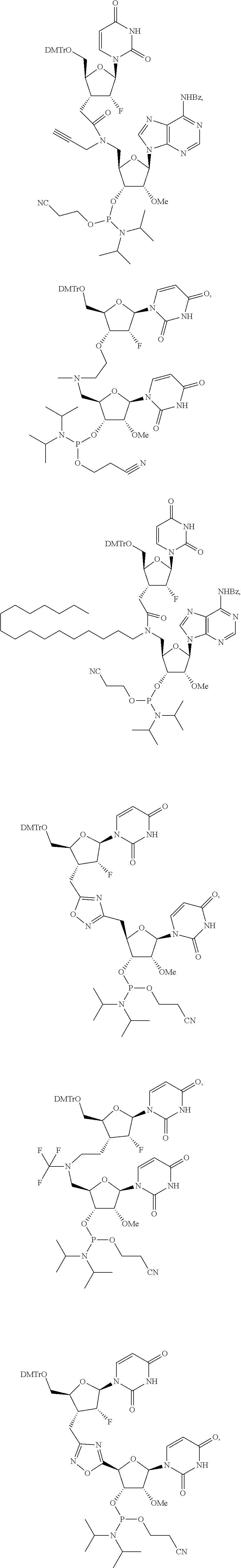

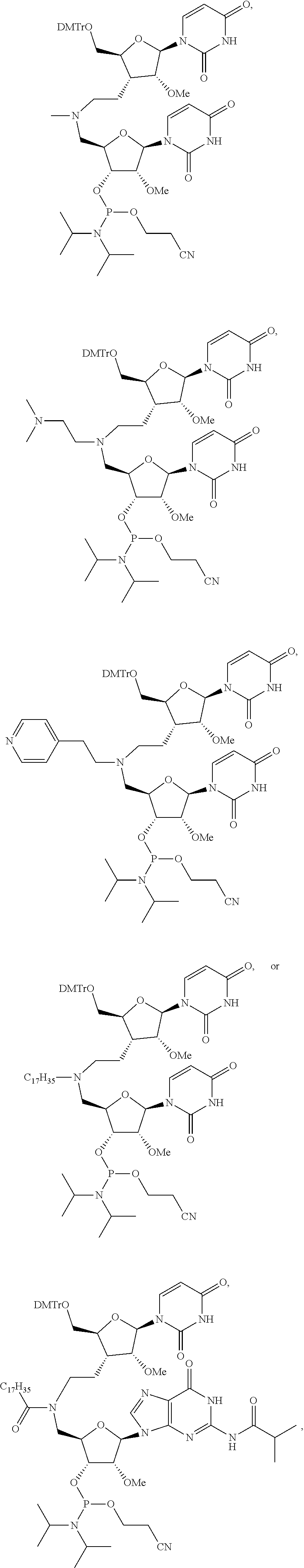

or a salt thereof.

In certain embodiments, an oligomeric compound is any of those described herein. In certain embodiments, the oligomeric compound is about 10-50 subunits in length. In certain embodiments the oligomeric compound is an oligonucleotide. In certain embodiments, an oligonucleotide is any of those described herein. In certain embodiments, the oligonucleotide is 8 to 80 linked nucleosides in length, 12-30 linked nucleosides in length, 12-30 linked nucleosides in length, or 15-30 linked nucleosides in length.

In certain embodiments, the compounds described herein are modified oligonucleotides. In certain embodiments, the compounds disclosed herein comprise modified oligonucleotides. In certain embodiments, the oligonucleotide is a modified oligonucleotide comprising at least one modified internucleoside linkage, at least one modified sugar, and/or at least one modified nucleobase.

In certain embodiments, the oligonucleotide is single-stranded. In certain embodiments, the oligonucleotide is double-stranded. In certain embodiments, the oligonucleotide comprises ribonucleic acids (e.g., comprised of ribonucleosides), deoxyribonucleic acids (e.g., comprised of deoxyribonucleosides), or a combination thereof. In certain embodiments, the oligonucleotide is a small interfering RNA (siRNA), a microRNA (miRNA) antagonist, an miRNA mimic, an ADAR recruiting molecule, an ADAR targeting molecule, a guide RNA, an antisense oligonucleotide, a short hairpin RNA (shRNA), or combinations thereof.

Certain embodiments provide a composition comprising a compound of any embodiment herein, and a pharmaceutically acceptable carrier or excipient.

Certain embodiments provide a composition comprising a compound of any embodiment herein, for use in therapy.

In certain embodiments, a method for delivering an agent to cell comprises contacting the cell with the compound of any embodiments herein, thereby delivering the agent to the cell. In certain embodiments, the cell is a brain cell. In certain embodiments the cell is a cell of the frontal cortex. In certain embodiments, the agent is a therapeutic agent or diagnostic agent. In certain embodiments, the cell is in an animal.

In certain embodiments, a method of modulating the expression of a nucleic acid target in a cell comprises contacting the cell with the compound of any embodiments herein, thereby modulating expression of the nucleic acid target in the cell. In certain embodiments, the cell is a brain cell. In certain embodiments the cell is a cell of the frontal cortex. In certain embodiments, the agent is a therapeutic agent or diagnostic agent. In certain embodiments, contacting the cell with the compound the compound of any embodiments herein inhibits expression of the nucleic acid target. In certain embodiments, the nucleic acid target is pre-mRNA, mRNA, non-coding RNA, or miRNA. In certain embodiments, the cell is in an animal.

In certain embodiments, a method of modulating the expression of a nucleic acid target in a subject comprises administering to the subject any of the compounds or compositions provided herein, thereby modulating expression of the nucleic acid target in the subject. In certain embodiments, the expression of the nucleic acid is modulated in a brain cell. In certain embodiments, the brain cell is a cell of the frontal cortex. In certain embodiments, the nucleic acid target is pre-mRNA, mRNA, non-coding RNA, or miRNA. In certain embodiments, the compound is administered to the subject intrathecally.

In certain embodiments, a method of treating or ameliorating a disease, disorder, or symptom thereof in a subject, comprises administering to the subject any of the compounds or compositions provided herein, thereby treating, preventing, or ameliorating a disease, disorder, or symptom in the subject. In certain embodiments, the disease, disorder, or symptom thereof is a central nervous system (CNS) disease, disorder, or symptom thereof. In certain embodiments, the disease, disorder, or symptom thereof is Alzheimer's disease, or a symptom thereof. In certain embodiments, the compound is administered to the subject intrathecally. In certain embodiments, the compound or composition is administered to the subject in a therapeutically effective amount.

Also provided herewith is the use of a compound as described herein for the manufacture of a medicament in the treatment of a disease or disorder.

In another aspect, the present disclosure provides methods for making any of the compounds provided herein, comprising one or more compounds and chemical transformations described herein.

### Certain Compounds Comprising an Oligonucleotide

In certain embodiment, compounds described herein comprise oligonucleotides. In certain embodiments, an oligonucleotide has a nucleobase sequence that is at least partially complementary to a target nucleic acid sequence (e.g., an expressed target nucleic acid within a cell). In some embodiments, the oligonucleotide, upon delivery to a cell expressing a target nucleic acid, is able to inhibit the expression of the underlying gene. The gene expression can be inhibited in vitro or in vivo. In certain embodiments, an oligonucleotide comprises one or more ribonucleic acids (e.g., one or more ribonucleosides), deoxyribonucleic acids (e.g., one or more deoxyribonucleosides), modified nucleic acids (e.g., one or more modified nucleobases, sugars, and/or phosphate groups), or a combination thereof. In some embodiments, an oligonucleotide comprises a ribonucleic acid (RNA). In some embodiments, an oligonucleotide comprises a deoxyribonucleic acid (DNA). In some embodiments, an oligonucleotide comprises a modification (e.g., modified nucleobase, modified sugar, or modified phosphate).

In certain embodiments, an oligonucleotide is single-stranded. In some embodiments, a single-stranded oligonucleotide is single-stranded RNA (ssRNA), ssDNA, or a ssRNA/DNA hybrid (e.g., a single-stranded oligonucleotide comprised of both ribonucleosides (modified or unmodified) and deoxyribonucleosides (modified or unmodified)). In some embodiments, an oligonucleotide is double-stranded (e.g., comprised of two single-stranded nucleic acids). Such double-stranded oligonucleotides comprise a first oligonucleotide having a region complementary to a target nucleic acid and a second oligonucleotide having a region complementary to the first oligonucleotide. The first and second oligonucleotides can be independently modified.

In some embodiments, an oligonucleotide is at least 2 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or more) nucleotides in length. In some embodiments, an oligonucleotide is at least 5 nucleotides in length. In some embodiments, an oligonucleotide is at least 10 nucleotides in length. In some embodiments, an oligonucleotide is at least 15 nucleotides in length. In some embodiments, an oligonucleotide is at least 16 nucleotides in length. In some embodiments, an oligonucleotide is at least 17 nucleotides in length. In some embodiments, an oligonucleotide is at least 18 nucleotides in length. In some embodiments, an oligonucleotide is at least 19 nucleotides in length. In some embodiments, an oligonucleotide is at least 20 nucleotides in length. In some embodiments, an oligonucleotide is at least 21 nucleotides in length. In some embodiments, an oligonucleotide is at least 22 nucleotides in length. In some embodiments, an oligonucleotide is at least 23 nucleotides in length. In some embodiments, an oligonucleotide is at least 24 nucleotides in length. In some embodiments, an oligonucleotide is at least 25 nucleotides in length. In some embodiments, an oligonucleotide is at least 26 nucleotides in length. In some embodiments, an oligonucleotide is at least 27 nucleotides in length. In some embodiments, an oligonucleotide is at least 28 nucleotides in length. In some embodiments, an oligonucleotide is at least 29 nucleotides in length. In some embodiments, an oligonucleotide is at least 30 nucleotides in length. In some embodiments, an oligonucleotide is at least 40 nucleotides in length. In some embodiments, an oligonucleotide is at least 50 nucleotides in length. In some embodiments, an oligonucleotide is at least 60 nucleotides in length. In some embodiments, an oligonucleotide is at least 70 nucleotides in length. In some embodiments, an oligonucleotide is at least 80 nucleotides in length. In some embodiments, an oligonucleotide is at least 90 nucleotides in length. In some embodiments, an oligonucleotide is at least 100 nucleotides in length. In some embodiments, an oligonucleotide is at least 150 nucleotides in length.

In some embodiments, an oligonucleotide is less than or equal to 150 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150) nucleotides in length. In some embodiments, an oligonucleotide is less than or equal to 150 nucleotides in length. In some embodiments, an oligonucleotide is less than or equal to 100 nucleotides in length. In some embodiments, an oligonucleotide is less than or equal to 90 nucleotides in length. In some embodiments, an oligonucleotide is less than or equal to 80 nucleotides in length. In some embodiments, an oligonucleotide is less than or equal to 70 nucleotides in length. In some embodiments, an oligonucleotide is less than or equal to 60 nucleotides in length. In some embodiments, an oligonucleotide is less than or equal to 50 nucleotides in length. In some embodiments, an oligonucleotide is less than or equal to 40 nucleotides in length. In some embodiments, an oligonucleotide is less than or equal to 30 nucleotides in length. In some embodiments, an oligonucleotide is less than or equal to 29 nucleotides in length. In some embodiments, an oligonucleotide is less than or equal to 28 nucleotides in length. In some embodiments, an oligonucleotide is less than or equal to 27 nucleotides in length. In some embodiments, an oligonucleotide is less than or equal to 26 nucleotides in length. In some embodiments, an oligonucleotide is less than or equal to 25 nucleotides in length. In some embodiments, an oligonucleotide is less than or equal to 24 nucleotides in length. In some embodiments, an oligonucleotide is less than or equal to 23 nucleotides in length. In some embodiments, an oligonucleotide is less than or equal to 22 nucleotides in length. In some embodiments, an oligonucleotide is less than or equal to 21 nucleotides in length. In some embodiments, an oligonucleotide is less than or equal to 20 nucleotides in length. In some embodiments, an oligonucleotide is less than or equal to 19 nucleotides in length. In some embodiments, an oligonucleotide is less than or equal to 18 nucleotides in length. In some embodiments, an oligonucleotide is less than or equal to 17 nucleotides in length. In some embodiments, an oligonucleotide is less than or equal to 16 nucleotides in length. In some embodiments, an oligonucleotide is less than or equal to 15 nucleotides in length. In some embodiments, an oligonucleotide is less than or equal to 10 nucleotides in length. In some embodiments, an oligonucleotide is less than or equal to 5 nucleotides in length.

In some embodiments, an oligonucleotide is about 5 nucleotides in length to about 150 nucleotides in length. In some embodiments, an oligonucleotide is about 10 nucleotides in length to about 100 nucleotides in length. In some embodiments, an oligonucleotide is about 20 nucleotides in length to about 90 nucleotides in length. In some embodiments, an oligonucleotide is about 30 nucleotides in length to about 80 nucleotides in length. In some embodiments, an oligonucleotide is about 40 nucleotides in length to about 70 nucleotides in length. In some embodiments, an oligonucleotide is about 50 nucleotides in length to about 60 nucleotides in length. In some embodiments, an oligonucleotide is about 15 nucleotides in length to about 30 nucleotides in length. In some embodiments, an oligonucleotide is about 18 nucleotides in length to about 25 nucleotides in length. In some embodiments, an oligonucleotide is about 19 nucleotides in length to about 23 nucleotides in length. In certain embodiments, the oligonucleotide is a modified oligonucleotide.

In some embodiments, an oligonucleotide is about 18 nucleotides in length to about 25 nucleotides in length.

In some embodiments, the double-stranded region of a double-stranded oligonucleotide is equal to or at least, 10, 11, 12, 13, 14, 15, 16, 17, 18, 19, 20, 21, 22, 23, 23, 24, 25, 26, 27, 28, 29, 30 or more nucleotide pairs in length.

In some embodiments, the antisense strand of a double-stranded oligonucleotide is equal to or at least 14, 15, 16, 17, 18, 19, 20, 21, 22, 23, 23, 24, 25, 26, 27, 28, 29, or 30 nucleotides in length.

In some embodiments, the sense strand of a double-stranded oligonucleotide is equal to or at least 10, 11, 12, 13, 14, 15, 16, 17, 18, 19, 20, 21, 22, 23, 23, 24, 25, 26, 27, 28, 29, or 30 nucleotides in length.

In some embodiments, an oligonucleotide is a therapeutic oligonucleotide. A therapeutic oligonucleotide may comprise, for example, without limitation, a small interfering RNA (siRNA), a microRNA (miRNA) antagonist, a miRNA mimic, an ADAR recruiting molecule, an ADAR targeting molecule, a guide RNA, an antisense oligonucleotide, a short hairpin RNA (shRNA), or combinations thereof.

In certain embodiments, a miRNA is a precursor, primary, and/or mature miRNA.

In certain embodiments, an oligonucleotide comprises or consists of an antisense oligonucleotide. In certain embodiments, an antisense oligonucleotide is complementary to an mRNA. In certain embodiments, an antisense oligonucleotide is complementary to a pre-mRNA. In certain embodiments, an antisense oligonucleotide blocks translation and promotes degradation of the mRNA transcript. In certain embodiments, an antisense oligonucleotide recruits Rnase H and promotes degradation of the mRNA transcript. In certain embodiments, an antisense oligonucleotide targets miRNA, inhibiting the miRNA from modulating mRNA expression and promoting degradation of the miRNA.

### Certain Modifications

In certain aspects, the disclosure relates to compounds that comprise oligonucleotides. In certain embodiments, oligonucleotides may be unmodified RNA or DNA or may be modified. In certain embodiments, the oligonucleotides are modified oligonucleotides. In certain embodiments, the modified oligonucleotides comprise at least one modified sugar, modified nucleobase, or modified internucleoside linkage relative to an unmodified RNA or DNA. In certain embodiments, an oligonucleotide has a modified nucleoside. A modified nucleoside may comprise a modified sugar, a modified nucleobase, or both a modified sugar and a modified nucleobase. Modified oligonucleotides may also include end modifications, e.g., 5′-end modifications and 3′-end modifications.

### Sugar Modifications and Motifs

In certain embodiments, a modified sugar is a substituted furanosyl sugar or non-bicyclic modified sugar. In certain embodiments, a modified sugar is a bicyclic or tricyclic modified sugar. In certain embodiments, a modified sugar is a sugar surrogate. A sugar surrogate may comprise one or more substitutions described herein.

In certain embodiments, a modified sugar is a substituted furanosyl or non-bicyclic modified sugar. In certain embodiments, the furanosyl sugar is a ribosyl sugar. In certain embodiments, the furanosyl sugar comprises one or more substituent groups, including, but not limited to, substituent groups at the 2′, 3′, 4′, and 5′ positions.

In certain embodiments, substituents at the 2′ position include, but are not limited to, F and OCH₃ (“OMe”, “O-methyl” or “methoxy”). In certain embodiments, substituent groups at the 2′ position suitable for non-bicyclic modified sugars include, but are not limited to, halo, allyl, amino, azido, SH, CN, OCN, CF₃, OCF₃, F, Cl, Br, SCH₃, SOCH₃, SO₂CH₃, ONO₂, NO₂, N₃, and NH₂. In certain embodiments, substituent groups at the 2′ position include, but are not limited to, O—(C₁-C₁₀) alkoxy, alkoxyalkyl, O-alkyl, S-alkyl, N-alkyl, O-alkenyl, S-alkenyl, N-alkenyl, O-alkynyl, S-alkynyl, N-alkynyl, O-alkyl-O-alkyl, alkynyl, wherein the alkyl, alkenyl and alkynyl can be substituted or unsubstituted C₁ to C₁₀ alkyl or C₂ to C₁₀ alkenyl and alkynyl. In certain embodiments, substituent groups at the 2′ position include, but are not limited to, alkaryl, aralkyl, O-alkaryl, and O-aralkyl. In certain embodiments, these 2′ substituent groups can be further substituted with one or more substituent groups independently selected from hydroxyl, alkoxy, carboxy, benzyl, phenyl, nitro (NO₂), thiol, thioalkoxy, thioalkyl, halogen, alkyl, aryl, alkenyl, and alkynyl. In certain embodiments, substituent groups at the 2′ position include, but are not limited to, O[(CH₂)ₙO]ₘCH₃, O(CH₂)ₙOCH₃, O(CH₂)ₙCH₃, O(CH₂)ₙONH₂, O(CH₂)ₙNH₂, O(CH₂)ₙSCH₃, and O(CH₂)ₙON[(CH₂)·CH₃)]₂, where n and m are independently from 1 to about 10. In certain embodiments, substituent groups at the 2′ position include, but are not limited to, OCH₂CH₂OCH₃ (“MOE”), O(CH₂)₂ON(CH₃)₂(“DMAOE”), O(CH₂)₂O(CH₂)₂N(CH₃)₂(“DMAEOE”), and OCH₂C(═O)—N(H)CH₃ (“NMA”).

In certain embodiments, substituent groups at the 4′ position suitable for non-bicyclic modified sugars include, but are not limited to, alkoxy (e.g., methoxy), alkyl, and those described in Manoharan et al., WO 2015/106128. In certain embodiments, substituent groups at the 5′ position suitable for non-bicyclic modified sugars include, but are not limited to, methyl (“Me”) (R or S), vinyl, and methoxy. In certain embodiments, the 5′ modification is a 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′adenosine cap (Appp), and any modified or unmodified nucleotide cap structure (N—O-5′(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′phosphorothiolate ((HO)₂(O)P—S-5′); any additional combination of oxygen/sulfur replaced monophosphate, diphosphate and triphosphates (e.g. 5′-alpha-thiotriphosphate, 5′-gammathiotriphosphate, etc.), 5′-phosphoramidates ((HO)₂(O)P—NH-5′, (HO)(NH₂)(O)P—O-5′), 5′alkylphosphonates (R=alkyl=methyl, ethyl, isopropyl, propyl, etc., e.g. RP(OH)(O)—O-5′-, 5′alkenylphosphonates (i.e. vinyl, substituted vinyl), (OH)₂(O)P-5′-CH₂—), 5′alkyletherphosphonates (R=alkylether=methoxymethyl (MeOCH₂—), ethoxymethyl, etc., e.g. RP(OH)(O)—O-5′-). In certain embodiments, one or more sugars comprise a 5′-vinylphosphonate modification. In certain embodiments, one or more sugars comprise a 5′-ethylenephosphonate modification. In certain embodiments the 5′ modification is at the terminus of an oligonucleotide. In certain embodiments the 5′ modification is at the terminus of an antisense oligonucleotide. In certain embodiments, substituents described herein for the 2′, 4′, and 5′ position can be added to other specific positions on the sugar. In certain embodiments, such substituents may be added to the 3′ position of the sugar on the 3′ terminal nucleoside or the 5′ position of the 5′ terminal nucleoside. In certain embodiments, a non-bicyclic modified sugar may comprise more than one non-bridging sugar substituent. In certain such embodiments, non-bicyclic modified sugars substituents include, but are not limited to, 5′-Me-2′-F, 5′-Me-2′-OMe (including both R and S isomers). In certain embodiments, modified sugar substituents include those described in Migawa et al., WO 2008/101157 and Rajeev et al., US2013/0203836.

In certain embodiments, a modified sugar is a bicyclic sugar. A bicyclic sugar is a modified sugar comprising two rings, wherein the second ring is formed via a bridge connecting two of the atoms in the first ring, thereby forming a bicyclic structure. In certain embodiments, a bicyclic sugar comprises a bridging substituent that bridges two atoms of the furanosyl ring to form a second ring. In certain embodiments, a bicyclic sugar does not comprise a furanosyl moiety. A “bicyclic nucleoside” (“BNA”) is a nucleoside having a bicyclic sugar. In certain embodiments, the bicyclic sugar comprises a bridge between the 4′ and 2′ furanose ring atoms. In certain embodiments, the bicyclic sugar comprises a bridge between the 5′ and 3′ furanose ring atoms. In certain such embodiments, the furanose ring is a ribose ring. In certain embodiments, 4′ to 2′ bridging substituents include, but are not limited to, 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (“constrained ethyl” or “cEt” when in the S configuration), 4′-CH₂O—CH₂-2′, 4′-CH₂—NI-2″ 4′-CH(CH₂OCH₃)-′-2′ (“constrained MOE” or “cMOE”) and analogs thereof (e.g., U.S. Pat. No. 7,399,845)′ 4′-C(CH₃)(CH₃)-′-2′ and analogs thereof (e.g., U.S. Pat. No. 8,278,283)′ 4′-CH₂—N(OCH₃′-2′ and analogs thereof (e.g., U.S. Pat. No. 8,278,425)′ 4′-CH₂—O—N(CH₃′-2′ (e.g., U.S. Patent Publication No. 2004/0171570)′ 4′-CH_{I}(′)—O-2′, wherein R is H, C₁-C₁₂ alkyl, or a protecting group (e.g., U.S. Pat. No. 7,427,6′2), 4′-CH₂—C(H)(CH₃)-2′ (e.g., Chattopadhyaya et al., J. Org. Chem., 2009, 74, 118-134), and 4′-CH₂—C(═CH₂)-2′ and analogs thereof (e.g., U.S. Pat. No. 8,278,426). The entire contents of each of the foregoing are hereby incorporated herein by reference. Additional representative U.S. patents and U.S. patent Publications that teach the preparation of bicyclic nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, US 2013/0190383; and WO 2013/036868, the entire contents of each of which are hereby incorporated herein by reference. Any of the foregoing bicyclic nucleosides can be prepared having one or more stereochemical sugar configurations including, for example, α-L-ribofuranose and β-D-ribofuranose (see, e.g., WO 99/14226). Specified bicyclic nucleosides herein are in the β-D configuration, unless otherwise specified.

In certain embodiments, a modified sugar is a sugar surrogate. In certain embodiments, a sugar surrogate has the oxygen atom replaced, e.g., with a sulfur, carbon or nitrogen atom. In certain such embodiments, the sugar surrogate may also comprise bridging and/or non-bridging substituents as described herein. In certain embodiments, sugar surrogates comprise rings having other than 5 atoms. In certain such embodiments, the sugar surrogate comprises a cyclobutyl moiety in place of the pentofuranosyl sugar. In certain embodiments, the sugar surrogate comprises a six membered ring in place of the pentofuranosyl sugar. In certain embodiments, the sugar surrogate comprises a tetrahydropyran (“THP”) in place of the pentofuranosyl sugar. In certain embodiments, the sugar surrogate comprises a morpholino in place of the pentofuranosyl sugar. Representative US patents that teach the preparation of such modified sugar structures include, but are not limited to, U.S. Pat. Nos. 4,981,957; 5,118,800; 5,166,315; 5,185,444; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,700,920; 7,875,733; 7,939,677, 8,088,904; 8,440,803; and 9,005,906, the entire contents of each of the foregoing are hereby incorporated herein by reference.

In some embodiments, sugar surrogates comprise acyclic moieties. In certain embodiments, the sugar surrogate is an unlocked nucleic acid (“UNA”). A UNA is unlocked acyclic nucleic acid, wherein any of the bonds of the sugar has been removed, forming an unlocked “sugar” residue. In one example, UNA also encompasses a monomer where the bonds between C1′-C4′ have been removed (i.e., the covalent carbon-oxygen-carbon bond between the C1′ and C4′ carbons). In another example, the C2′-C3′ bond (i.e., the covalent carbon-carbon bond between the C2′ and C3′ carbons) of the sugar has been removed. Representative U.S. publications that teach the preparation of UNA include, but are not limited to, U.S. Pat. No. 8,314,227; and U.S. Patent Publication Nos. 2013/0096289; 2013/0011922; and 2011/0313020, the entire contents of each of which are hereby incorporated herein by reference. In certain embodiments, sugar surrogates comprise peptide nucleic acid (“PNA”), acyclic butyl nucleic acid (BuNA) (see, e.g., Kumar et al., Org. Biomol. Chem., 2013, 11, 5853-5865), glycol nucleic acid (GNA) (see, e.g., Zhang et al, J. Am. Chem. Soc., 2005, 127 (12) 4174-4175), threoninol nucleic acid (TNA) (see, e.g., Asanuma et al., J. Am. Chem. Soc., 2010, 132 (42) 14702-14703) or analogs thereof, and nucleosides and oligonucleotides described in Manoharan et al., US2013/130378, the entire contents of which is hereby incorporated herein by reference. Many other bicyclic and tricyclic sugar and sugar surrogate ring systems are known in the art that can be used in modified nucleosides.

In certain aspects, the disclosure relates to compounds comprising at least one oligonucleotide wherein the nucleosides of such oligonucleotide comprise one or more types of modified sugars and/or unmodified sugars arranged along the oligonucleotide or region thereof in a defined pattern or “sugar motif”. In certain instances, such sugar motifs include, but are not limited to, any of the patterns of sugar modifications described herein.

In certain embodiments, an oligonucleotide comprises a gapmer sugar motif. A gapmer oligonucleotide comprises or consists of a region having two external “wing” regions and a central or internal “gap” region. The gap and wing regions form a contiguous sequence of nucleosides, wherein the majority of nucleoside sugars of each of the wings differ from the majority of nucleoside sugars of the gap. In certain embodiments, the wing regions comprise a majority of modified sugars and the gap comprises a majority of unmodified sugars. In certain embodiments, the nucleosides of the gap are deoxynucleosides. Compounds with a gapmer sugar motif are described in, for example, U.S. Pat. No. 8,790,919, the entire contents of which is hereby incorporated herein by reference.

In certain embodiments, one or both oligonucleotides of a double-stranded compound comprise a triplet sugar motif. An oligonucleotide with a triplet sugar motif comprises three identical sugar modifications on three consecutive nucleosides. In certain embodiments, the triplet is at or near the cleavage site of the oligonucleotide. In certain embodiments, an oligonucleotide of a double-stranded compound may contain more than one triplet sugar motif. In certain embodiments, the identical sugar modification of the triplet sugar motif is a 2′-F modification. Compounds with a triplet sugar motif are disclosed, for example, in U.S. Pat. No. 10,668,170, the entire contents of which is incorporated herein by reference.

In certain embodiments, one or both oligonucleotides of a double-stranded compound comprise a quadruplet sugar motif. An oligonucleotide with a quadruplet sugar motif comprises four identical sugar modifications on four consecutive nucleosides. In certain embodiments, the quadruplet is at or near the cleavage site. In certain embodiments, an oligonucleotide of a double-stranded compound may contain more than one quadruplet sugar motif. In certain embodiments, the identical sugar modification of the quadruplet sugar motif is a 2′-F modification. For a double-stranded compound having a duplex region of 19-23 nucleotides in length, the cleavage site of the antisense oligonucleotide is typically around the 10, 11, and 12 positions from the 5′-end. In certain embodiments, the quadruplet sugar motif is at the 8, 9, 10, 11 positions; the 9, 10, 11, 12 positions; the 10, 11, 12, 13 positions; the 11, 12, 13, 14 positions; or the 12, 13, 14, 15 positions of the sense oligonucleotide, counting from the first nucleoside of the 5′-end of the sense oligonucleotide, or, the count starting from the first paired nucleotide within the duplex region from the 5′-end of the sense oligonucleotide. In certain embodiments, the quadruplet sugar motif is at the 8, 9, 10, 11 positions; the 9, 10, 11, 12 positions; the 10, 11, 12, 13 positions; the 11, 12, 13, 14 positions; or the 12, 13, 14, 15 positions of the antisense oligonucleotide, counting from the first nucleoside of the 5′-end of the antisense oligonucleotide, or, the count starting from the first paired nucleotide within the duplex region from the 5′-end of the antisense oligonucleotide. The cleavage site may change according to the length of the duplex region of the double-stranded compound and may change the position of the quadruplet accordingly.

In certain embodiments, an oligonucleotide comprises an alternating sugar motif. In certain embodiments, one or both oligonucleotides of a double-stranded compound comprise an alternating sugar motif. An oligonucleotide with an alternating sugar motif comprises at least two different sugar modifications, wherein one or more consecutive nucleosides comprising a first sugar modification alternates with one or more consecutive nucleosides comprising a second sugar modification, and one or more consecutive nucleosides comprising a third sugar modification, etc. For example, if A, B, and C each represent one type of modification to the nucleoside, the alternating motif can be “ABABABABABAB . . . ,” “AABBAABBAABB . . . ,” “AABAABAABAAB “AAABAAABAAAB . . . ,” “AAABBBAAABBB . . . ,” or “ABCABCABCABC . . . ” etc. In certain embodiments, the alternating sugar motif is repeated for at least 2, 3, 4, 5, 6, 7, 8, 9, 10, 11, 12, 13, 14, 15, 16, 17, 18, 19, 20, 21, 22, or 23 contiguous nucleobases along an oligonucleotide. In certain embodiments, the alternating sugar motif is comprised of two different sugar modifications. In certain embodiments, the alternating sugar motif comprises 2′-OMe and 2′-F sugar modifications.

In certain embodiments, each nucleoside of an oligonucleotide is independently modified with one or more sugar modifications provided herein. In certain embodiments, each oligonucleotide of a double-stranded compound independently has one or more sugar motifs provided herein. In certain embodiments, an oligonucleotide containing a sugar motif is fully modified in that each nucleoside other than the nucleosides comprising the sugar motif comprises a sugar modification.

### Nucleobase Modifications and Motifs

In certain embodiments, modified oligonucleotides comprise one or more nucleosides comprising a modified nucleobase. In certain embodiments, modified oligonucleotides comprise one or more nucleosides that do not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly, 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size expanded bases, and fluorinated bases. Further modified nucleobases include tricyclic pyrimidines, such as 1,3-diazaphenoxazine-2-one, 1,3-diazaphenothiazine-2-one, and 9-(2-aminoethoxy)-1,3-diazaphenoxazine-2-one (G-clamp). Modified nucleobases may also include those in which the purine or pyrimidine base is replaced with other heterocycles, for example, 7-deaza-adenine, 7-deazaguanosine, 2-aminopyridine and 2-pyridone.

Further nucleobases include those disclosed in U.S. Pat. No. 3,687,808; Modified Nucleosides in Biochemistry, Biotechnology and Medicine, Herdewijn, P. ed. Wiley-VCH, 2008; The Concise Encyclopedia Of Polymer Science And Engineering, pages 858-859; Kroschwitz, J. L., Ed., John Wiley & Sons, 1990, 858-859; Englisch et al., Angewandte Chemie, International Edition, 1991, 30, 613; Sanghvi, Y. S., Chapter 15, dsRNA Research and Applications, pages 289-302; Antisense Research and Applications, Crooke, S. T. and Lebleu, B., Eds., CRC Press, 1993, 273-288; Antisense Drug Technology, Crooke S. T., Ed., CRC Press, 2008, 163-166 and 442-443 (Chapters 6 and 15), each of which are hereby incorporated herein by reference.

Publications that teach the preparation of certain of the above noted modified nucleobases, as well as other modified nucleobases include without limitation, U.S. Applications 2003/0158403 and 2003/0175906; U.S. Pat. Nos. 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121; 5,596,091; 5,614,617; 5,645,985; 5,681,941; 5,811,534; 5,750,692; 5,948,903; 5,587,470; 5,457,191; 5,763,588; 5,830,653; 5,808,027; 6,005,096. 6,015,886; 6,147,200; 6,166,197; 6,166,199; 6,222,025; 6,235,887; 6,380,368; 6,528,640; 6,639,062; 6,617,438; 7,045,610; 7,427,672; and 7,495,088, the entire contents of each of which are hereby incorporated herein by reference.

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments, the block is within 3 nucleosides of the 3′-end of the oligonucleotide. In certain embodiments, the block is at the 5′-end of the oligonucleotide. In certain embodiments, the block is within 3 nucleosides of the 5′5′-end of the oligonucleotide.

### Internucleoside Linkage Modifications and Motifs

A 3′ to 5′ phosphodiester linkage is the naturally occurring internucleoside linkage of RNA and DNA. In certain embodiments, an oligonucleotide has one or more modified, i.e., non-naturally occurring, internucleoside linkages. Certain non-naturally occurring internucleoside linkages may impart desirable properties such as, for example, enhanced cellular uptake, enhanced affinity for target nucleic acids, and increased stability in the presence of nucleases. Representative phosphorus-containing modified internucleoside linkages include, but are not limited to, phosphotriesters, alkylphosphonates (e.g., methylphosphonates), phosphoramidates, and phosphorothioates (“P═S”), and phosphorodithioates (“HS—P═S”). Representative non-phosphorus containing internucleoside linking groups include, but are not limited to, methylenemethylimino (—CH₂—N(CH₃)—O—CH₂), thiodiester, thionocarbamate (—O—C(═O)(NH)—S—); siloxane (—O—SiH₂—O—); and N,N′-dimethylhydrazine (—CH₂—N((CH₃)—N((CH₃)—). Methods of preparation of phosphorous-containing and non-phosphorous-containing internucleoside linkages are well known to those skilled in the art. Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See, for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, an oligonucleotide comprises at least one modified internucleoside linkage. A modified internucleoside linkage may be placed at any position of an oligonucleotide. For double-stranded compounds, a modified internucleoside linkage may be placed within the sense oligonucleotide, antisense oligonucleotide, or both oligonucleotides of the double-stranded compound.

In certain embodiments, the internucleoside linkage modification may occur on every nucleoside of an oligonucleotide. In certain embodiments, internucleoside linkage modifications may occur in an alternating pattern along an oligonucleotide. In certain embodiments, essentially each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the pattern of the internucleoside linkage modification on each oligonucleotide of a double-stranded compound is the same. In certain embodiments, the pattern of the internucleoside linkage modification on each oligonucleotide of a double-stranded compound is different. In certain embodiments, a double-stranded compound comprises 6-8 modified internucleoside linkages. In certain embodiments, the 6-8 modified internucleoside linkages are phosphorothioate internucleoside linkages or alkylphosphonate internucleoside linkages. In certain embodiments, the sense oligonucleotide comprises at least two modified internucleoside linkages at either or both the 5′-end and the 3′-end. In certain such embodiments, the modified internucleoside linkages are phosphorothioate internucleoside linkages or alkylphosphonate internucleoside linkages. In certain embodiments, the antisense oligonucleotide comprises at least two modified internucleoside linkages at either or both the 5′-end and the 3′-end. In certain such embodiments, the modified internucleoside linkages are phosphorothioate internucleoside linkages or alkylphosphonate internucleoside linkages.

In certain embodiments, a double-stranded compound comprises an overhang region. In certain embodiments, a double-stranded compound comprises a phosphorothioate or alkylphosphonate internucleoside linkage modification in the overhang region. In certain embodiments, a double-stranded compound comprises a phosphorothioate or alkylphosphonate internucleotide linkage linking the overhang nucleotide with a paired nucleotide that is next to the overhang nucleotide. For instance, there may be at least two phosphorothioate internucleoside linkages between the terminal three nucleosides, in which two of the three nucleosides are overhang nucleosides, and the third is a paired nucleoside next to the overhang nucleoside. These terminal three nucleosides may be at the 3′-end of the antisense oligonucleotide, the 3′-end of the sense oligonucleotide, the 5′-end of the antisense oligonucleotide, or the 5′end of the antisense oligonucleotide.

In certain embodiments, modified oligonucleotides comprise one or more internucleoside linkages having chiral centers. Representative chiral internucleoside linkages include, but are not limited to, alkylphosphonates and phosphorothioates. Modified oligonucleotides comprising internucleoside linkages having chiral centers can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. As is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration.

### Linkers

A “linker” refers to any chemical moiety (e.g., a combination of atoms having appropriate valency according to known chemistry principles) used to conjugate two components of the compounds provided herein (e.g., an oligonucleotide) to one another. Each of the two components may be connected to any portion of any of the linkers provided herein. In some embodiments, one component of the compounds provided herein (e.g., an oligonucleotide) is connected by a bond to one end of a linker, and the other component is connected by a bond to the other end of the linker. In some embodiments, one or both components of the compounds provided herein may be connected by a bond to an internal position within any of the linkers described herein. In some embodiments, a linker is a bond (including, e.g., phosphodiester and phosphorothioate bonds). In some embodiments, a linker is an substituted or unsubstituted alkyl linker (i.e., an alkyl chain is used to join two moieties, which may each be conjugated to opposite ends of the alkyl linker, or one or both moieties may be conjugated to an internal carbon on the alkyl linker). In some embodiments, a linker is an substituted or unsubstituted polyethylene glycol (PEG) linker (i.e., a PEG chain is used to join two moieties, which may each be conjugated to opposite ends of the PEG linker, or one or both moieties may be conjugated to an internal position on the PEG linker). In some embodiments, a linker is an substituted or unsubstituted heteroalkyl linker (i.e., a heteroalkyl chain is used to join two moieties, which may each be conjugated to opposite ends of the heteroalkyl linker, or one or both moieties may be conjugated to an internal position on the heteroalkyl linker). In some embodiments, a linker is an substituted or unsubstituted heteroaryl linker (i.e., a heteroaryl group is used to join two moieties, which may each be conjugated to any position on the heteroaryl group).

In some embodiments, a linker is of the formula

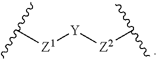

In certain embodiments, a linker is of the formula:

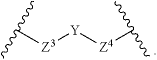

In some embodiments, a linker is a bond. In some embodiments, a linker is an substituted or unsubstituted PEG linker. In some embodiments, a linker is three or four PEG units in length. In certain embodiments, a linker comprises the structure

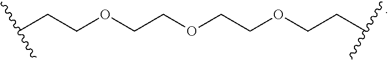 [SMILES: *CCOCCOCCOCC*]

In some embodiments, a linker is two or three PEG units in length.

In some embodiments, a linker is an substituted or unsubstituted heteroaryl linker. In some embodiments, a linker is an substituted or unsubstituted partially unsaturated heteroaryl linker. In some embodiments, a linker comprises the structure

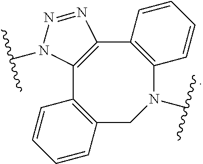 [SMILES: *N1Cc2ccccc2-c2c(nnn2*)-c2ccccc21]

In some embodiments, a linker is an substituted or unsubstituted heteroalkyl linker. In some embodiments, a linker is substituted with one or more ═O substituents. In certain embodiments, a linker comprises the structure

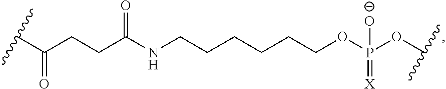

wherein X is O or S.

In some embodiments, a linker comprises the structure

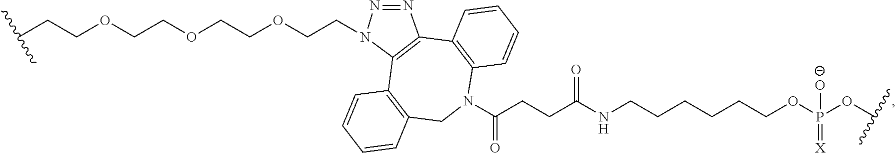

wherein X is O or S.

In some embodiments, a linker is a phosphodiester bond or a phosphorothioate bond. In certain embodiments, a linker comprises the structure

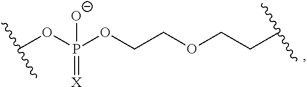

wherein X is O or S.

In certain embodiments, a linker comprises the structure

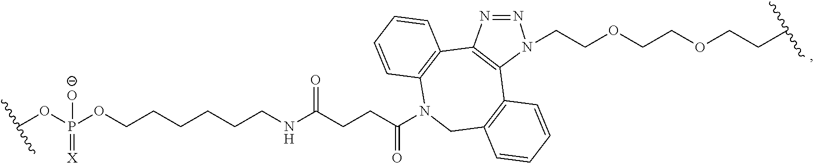

wherein X is O or S.

In some embodiments, a linker is an substituted or unsubstituted PEG linker. In some embodiments, a linker is an substituted or unsubstituted PEG linker three PEG units in length. In some embodiments, a linker is an substituted or unsubstituted heteroalkyl linker. In some embodiments, the heteroalkyl linker is substituted with one or more ═O substituents. In certain embodiments, a linker comprises the structure

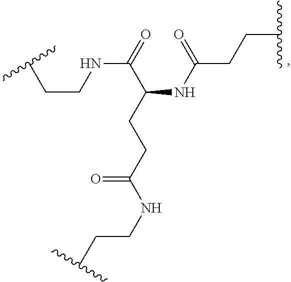 [SMILES: *CCNC(=O)CC[C@H](NC(=O)CC*)C(=O)NCC*]

In some embodiments, a linker is an substituted or unsubstituted heteroaryl linker. In some embodiments, a linker is an substituted or unsubstituted partially unsaturated heteroaryl linker. In certain embodiments, a linker comprises the structure

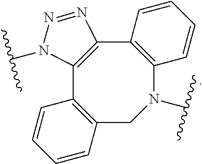 [SMILES: *N1Cc2ccccc2-c2c(nnn2*)-c2ccccc21]

In some embodiments, a linker is an substituted or unsubstituted heteroalkyl linker. In some embodiments, the heteroalkyl linker is substituted with one or more ═O substituents. In certain embodiments, a linker comprises the structure

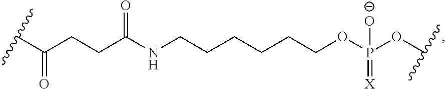

wherein X is o or S. In some embodiments, a linker comprises the structure

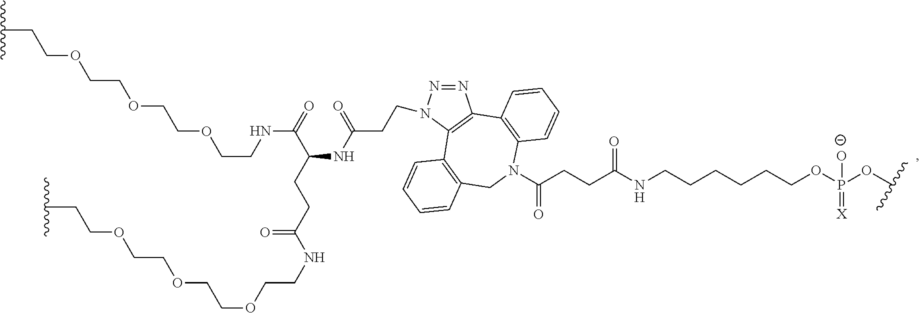

wherein X is O or S.

In some embodiments, a linker is an substituted or unsubstituted PEG linker. In certain embodiments, a linker is an substituted or unsubstituted PEG linker two or three PEG units in length. In some embodiments, a linker is an substituted or unsubstituted PEG linker. In some embodiments, a linker is an substituted or unsubstituted PEG linker three or four PEG units in length. In certain embodiments, a linker comprises the structure

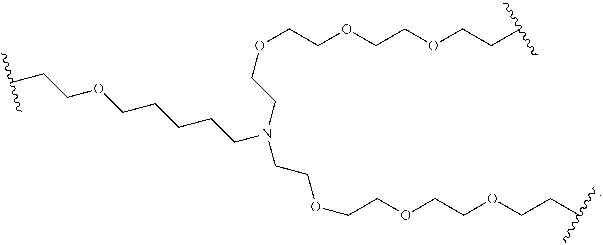 [SMILES: *CCOCCCCCN(CCOCCOCCOCC*)CCOCCOCCOCC*]

In some embodiments, a linker is an substituted or unsubstituted heteroaryl linker. In some embodiments, a linker is an substituted or unsubstituted partially unsaturated heteroaryl linker. In certain embodiments, a linker comprises the structure

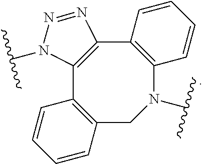 [SMILES: *N1Cc2ccccc2-c2c(nnn2*)-c2ccccc21]

In some embodiments, a linker is an substituted or unsubstituted heteroalkyl linker. In some embodiments, the heteroalkyl linker is substituted with one or more ═O substituents. In certain embodiments, a linker comprises the structure

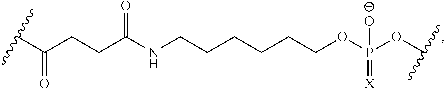

wherein X is O or S. In some embodiments, a linker comprises the structure

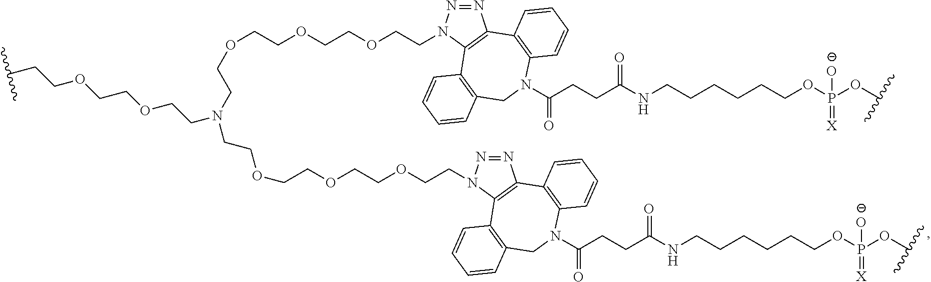

wherein X is O or S.

In some embodiments, X is O. In some embodiments, X is S.

In some embodiments, R⁴ and R⁵ comprise an oligonucleotide. In some embodiments, the oligonucleotide is attached at its 5′ end. In some embodiments, the oligonucleotide is attached at its 3′ end. In some embodiments, the oligonucleotide is attached at an internal position on the oligonucleotide. In some embodiments the internal position is at an internucleoside linkage. In certain embodiments, the oligonucleotide is a modified oligonucleotide.

In certain embodiments, the compounds disclosed herein are in salt form. In certain embodiments, the salt is a sodium salt. In certain embodiments, the salt is a potassium salt.

In certain embodiments, the compounds provided herein comprise one or more linking groups. In certain embodiments, each of L₁, L₂, L₃, and/or L₄ comprises a linking group. In certain embodiments, each of L₁, L₂, L₃, L₄, and/or L₅ comprises a linking group. In certain embodiments, each of L₁, L₂, L₃, L₄, L₅, L₆, and/or L₇ comprises a linking group. In certain embodiments, a linking group is covalently bound to an oligonucleotide. In certain embodiments, a linking group is covalently bound to a cleavable moiety. In certain embodiments, a linking group comprises a cleavable bond. In certain embodiments, a linking group does not comprise a cleavable moiety. In certain embodiments, a linking group comprises a covalent attachment to a solid support.

In certain embodiments, a linking group comprises a chain structure, such as a hydrocarbyl chain, or an oligomer of repeating units or combination of such repeating units. In certain embodiments, a linking group comprises 1 to 50 repeating units, 1 to 40 repeating units, 1 to 25 repeating units, 1 to 20 repeating units, 1 to 15 repeating units, 1 to 10 repeating units, or 1 to 5 repeating units. In certain embodiments, a linking group is 1 to 50 atoms long, 1 to 40 atoms long, 1 to 25 atoms long, 1 to 20 atoms long, 1 to 15 atoms long, 1 to 10 atoms long, or 1 to 5 atoms long.

In certain embodiments, a linking group contains carbon atoms. In certain embodiments, a linking group contains heteroatoms (e.g., nitrogen, oxygen, sulfur, etc.). In certain embodiments, a linking group forms amide linkages, ester linkages, or disulfide linkages. In certain embodiments, a linking group forms hydrazone linkages, oxime linkages, imine linkages, guanidine linkages, urea linkages, carbamate linkages, unsaturated alkyl linkages, sulfonamide linkages or 4-8 membered hetero cyclic linkages. In certain embodiments, a linking group comprises one or more groups selected from alkyl, amino, oxo, amide, disulfide, polyethylene glycol, ether, thioether, and hydroxylamino. In certain embodiments, a linking group comprises at least one phosphorus group. In certain embodiments, a linking group comprises at least one phosphate group. In certain embodiments, a linking group includes at least one neutral linking group. In certain embodiments, a linking group is substituted with various substituents including, but not limited to, hydrogen atoms, alkyl, alkenyl, alkynyl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. As would be appreciated by one of skill in this art, each of these groups may in turn be substituted.

In certain embodiments, a linking group includes, but is not limited to, substituted or unsubstituted C₁-C₁₀ alkylene, substituted or unsubstituted C₂-C₁₀ alkenylene, or substituted or unsubstituted C₂-C₁₀ alkynylene, wherein a nonlimiting list of preferred substituent groups includes hydroxyl, amino, alkoxy, carboxy, benzyl, phenyl, nitro, thiol, thioalkoxy, halogen, alkyl, aryl, alkenyl, and alkynyl. In certain embodiments, a linking group is an aliphatic or heteroaliphatic. For example, the linking group can a polyalkyl linking group. The linking group can be a polyether linking group. The linking group can be a polyethylene linking group, such as PEG.

In certain embodiments, the linking group is a short peptide chain. In certain embodiments, a linking group comprises 1 to 40 amino acids, 1 to 25 amino acids, 1 to 20 amino acids, 1 to 15 amino acids, 1 to 10 amino acids, or 1 to 5 amino acids.

In certain embodiments, a linking group comprises linker-nucleosides. In certain embodiments, a linking group comprises 1 to 40 linker-nucleosides, 1 to 25 linker-nucleosides, 1 to 20 linker-nucleosides, 1 to 15 linker-nucleosides, 1 to 10 linker-nucleosides, or 1 to 5 linker-nucleosides. In certain embodiments, such linker-nucleosides may be modified or unmodified nucleosides. It is typically desirable for linker-nucleosides to be cleaved from the compound after it reaches a target tissue. Accordingly, linker-nucleosides herein can be linked to one another and to the remainder of the compound through cleavable bonds. Herein, linker-nucleosides are not considered to be part of an oligonucleotide payload. Accordingly, in embodiments in which a compound comprises an oligonucleotide consisting of a specified number or range of linked nucleosides and/or a specified percent complementarity to a reference nucleic acid, those linker-nucleosides are not counted toward the length of the oligonucleotide and are not used in determining the percent complementarity of the oligonucleotide for the reference nucleic acid.

In certain embodiments, the linking group includes a protein binding group. In certain embodiments, the protein binding group is a lipid such as for example including but not limited to cholesterol, cholic acid, adamantane acetic acid, 1-pyrene butyric acid, dihydrotestosterone, 1,3-bis-O(hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, 03-(oleoyl)cholenic acid, dimethoxytrityl, or phenoxazine), a vitamin (e.g., folate, vitamin A, vitamin E, biotin, pyridoxal), a peptide, a carbohydrate (e.g., monosaccharide, disaccharide, trisaccharide, tetrasaccharide, oligosaccharide, polysaccharide), an endosomolytic component, a steroid (e.g., uvaol, hecigenin, diosgenin), a terpene (e.g., triterpene, e.g., sarsasapogenin, friedelin, epifriedelanol derivatized lithocholic acid), or a cationic lipid. In certain embodiments, the protein binding group is a C₁₆ to C₂₂ long chain saturated or unsaturated fatty acid, cholesterol, cholic acid, vitamin E, adamantane or 1-pentafluoropropyl.

In certain embodiments, a linking group includes, but is not limited to, pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA).

In certain embodiments, a linking group includes, without limitation, those linking groups described in the following references: U.S. Pat. Nos. 5,994,517; 6,300,319; 6,660,720; 6,906,182; 7,262,177; 7,491,805; 8,106,022; 7,723,509; 9,127,276; US 2006/0148740; US 2011/0123520; WO2013/033230; WO2012/037254, Biessen et al., J. Med. Chem. 1995, 38, 1846-1852; Lee et al., Bioorganic & Medicinal Chemistry 2011, 19, 2494-2500; Rensen et al., J. Biol. Chem. 2001, 276, 37577-37584; Rensen et al., J. Med. Chem. 2004, 47, 5798-5808; Sliedregt et al., J. Med. Chem. 1999, 42, 609-618; Valentijn et al., Tetrahedron, 1997, 53, 759-770; Lee, Carhohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycohiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromh Vase Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 7,582,744; 8,552,163; 8,137,695; 6,908,903; 6,383,812; 7,262,177; 6,525,031; 5,994,517; 6,660,720; 6,300,319; 7,723,509; 8,106,022; 7,491,805; 7,491,805; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. patent Application Publications US2011/0097264; US2011/0097265; US2013/0004427; US2003/0119724; US2011/0207799; US2012/0035115; US2012/0230938; US2005/0164235; US2006/0183886; US2012/0136042; US2012/0095075; US2013/0109817; US2006/0148740; US2008/0206869; US2012/0165393; US2012/0101148; US2013/0121954; US2011/0123520; US2003/0077829; US2008/0108801; and US2009/0203132; each of which is incorporated herein by reference in its entirety.

In certain embodiments, L₁, L₂, L₃, and L₄ (or L₁, L₂, L₃, L₄, and L₅, or L₁, L₂, L₃, L₄, L₅, L₆, and L₇) independently comprise or together comprise a structure selected from among:

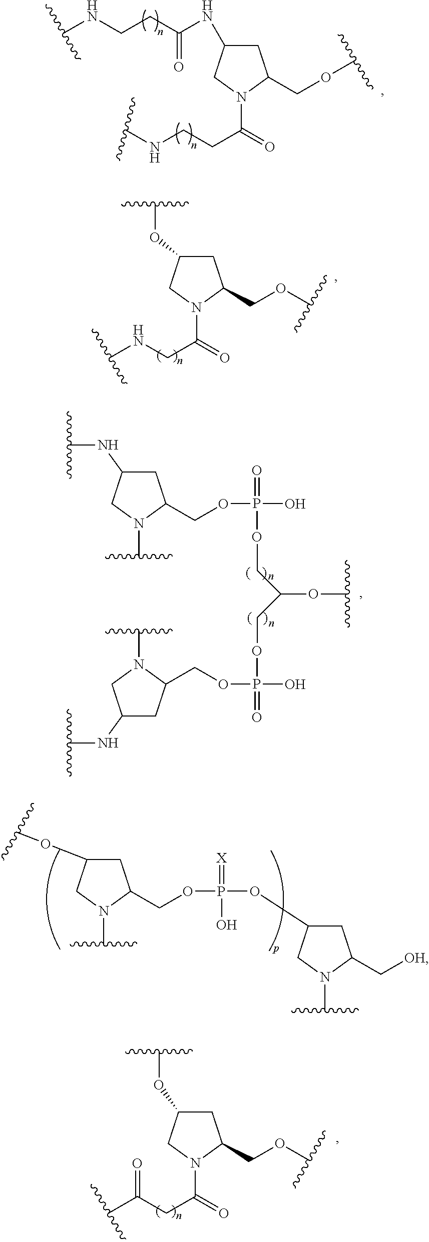

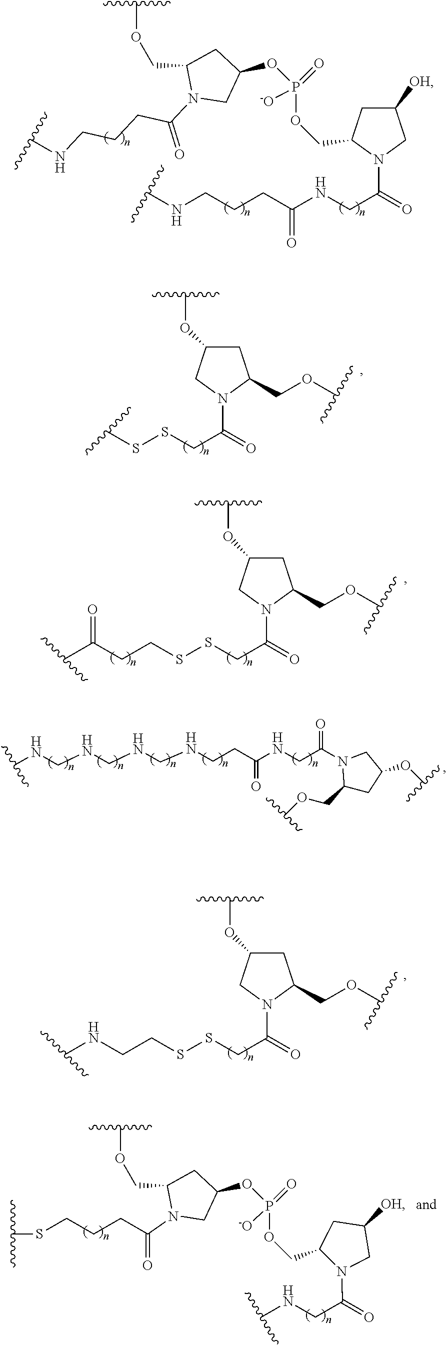

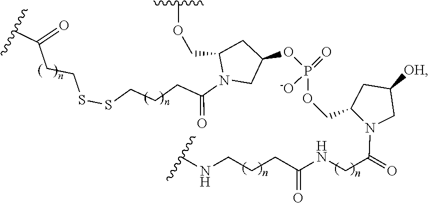

wherein each n is, independently, from 1 to 20; and p is from 1 to 6.

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among:

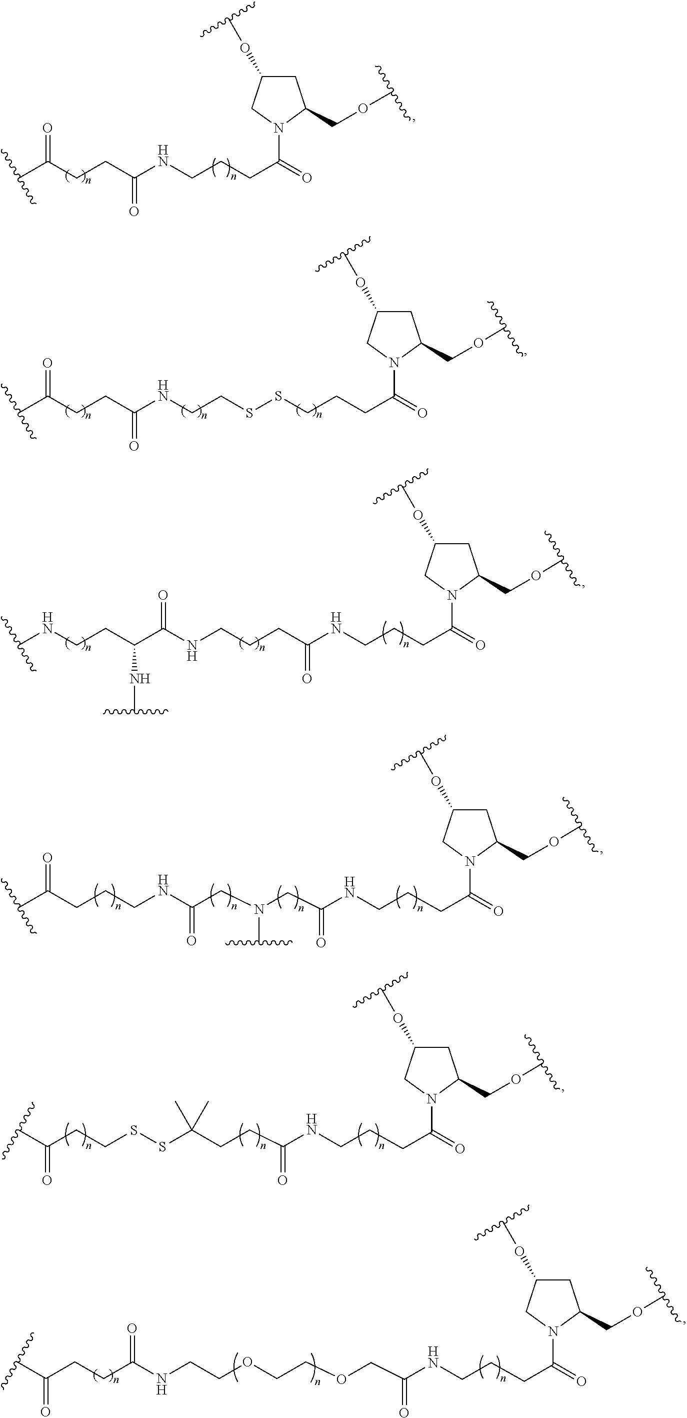

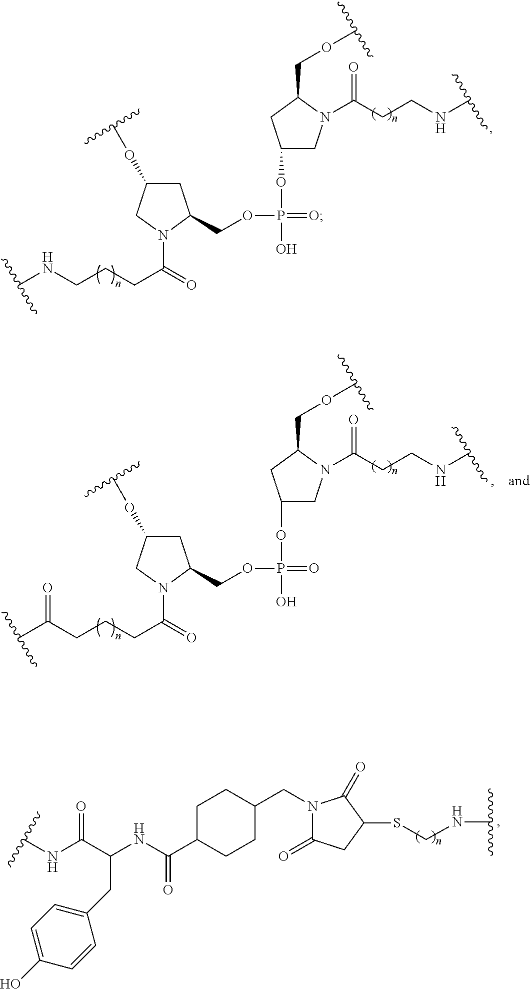

wherein each n is, independently, from 1 to 20.

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among:

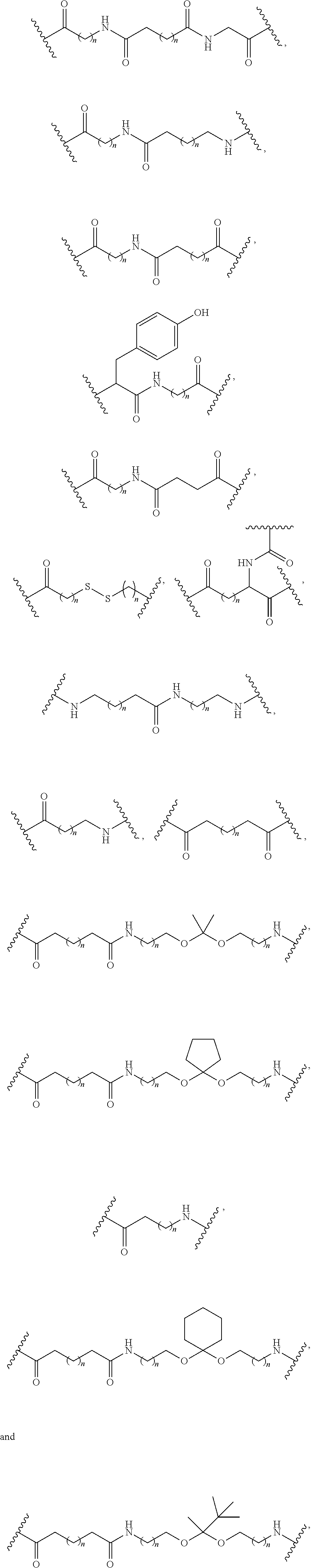

wherein each n is, independently, from 1 to 20.

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among:

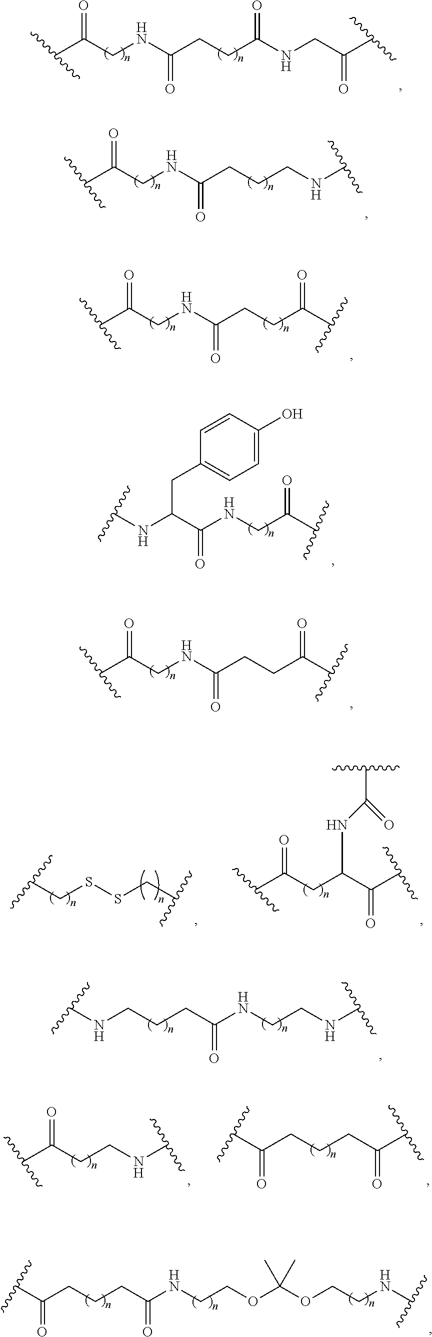

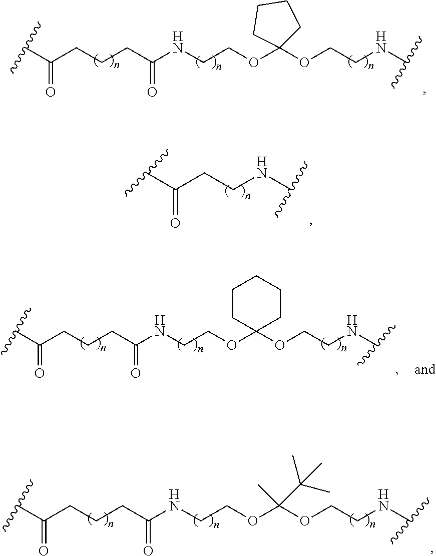

wherein each n is, independently, from 1 to 20.

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among:

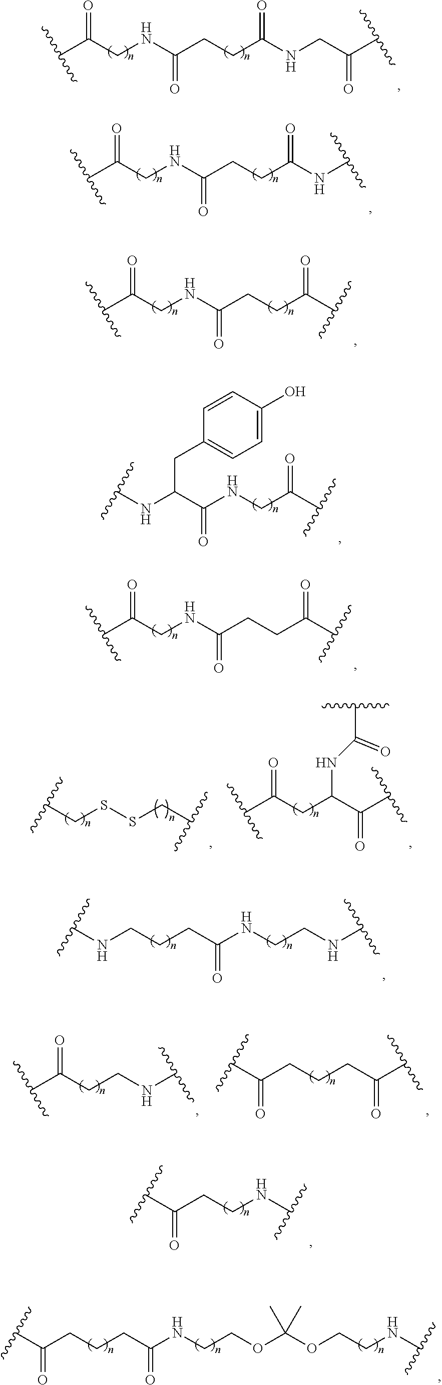

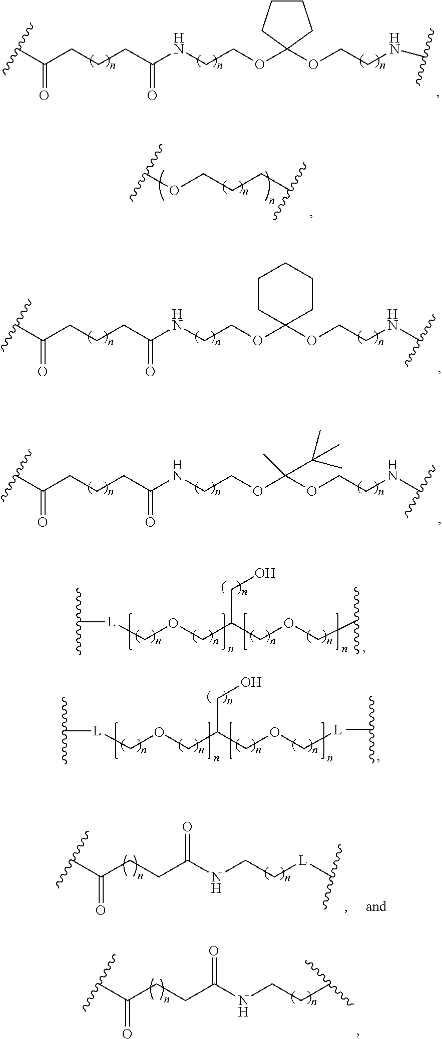

wherein each L is, independently, a phosphorous linking group; and each n is, independently, from 1 to 20.

In certain embodiments, each of L₁, L₂, L₃, L₄ L₅, L₆, and/or L₇ independently comprise or taken together comprise a structure selected from among:

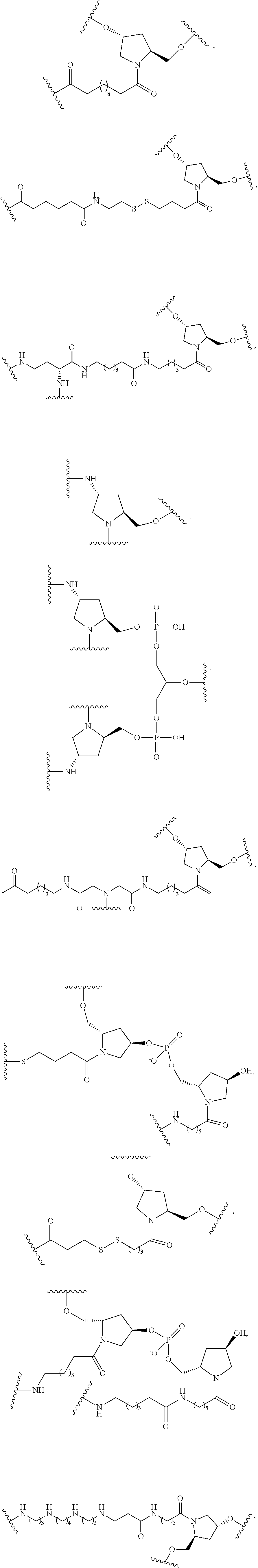

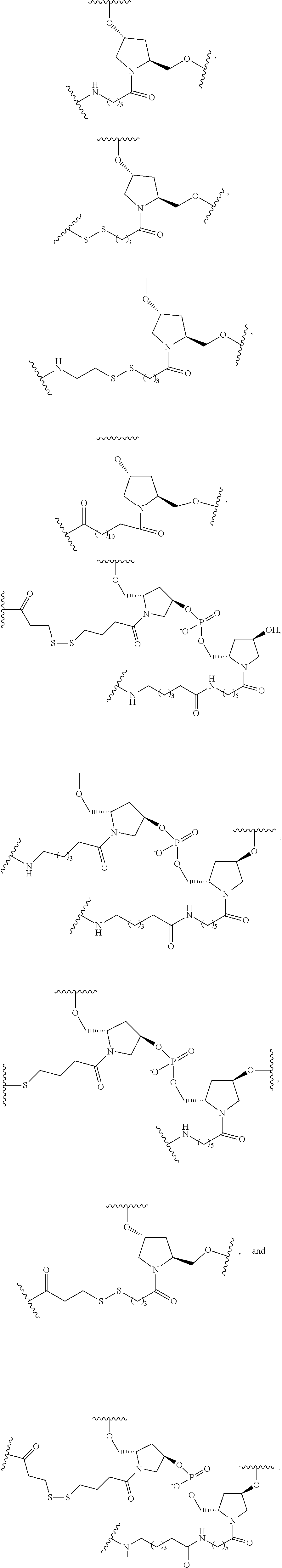

In certain embodiments, L₁, L₂, L₃, L₄, L₅, L₆, and/or L₇ independently comprise, or taken together comprise, a structure selected from among:

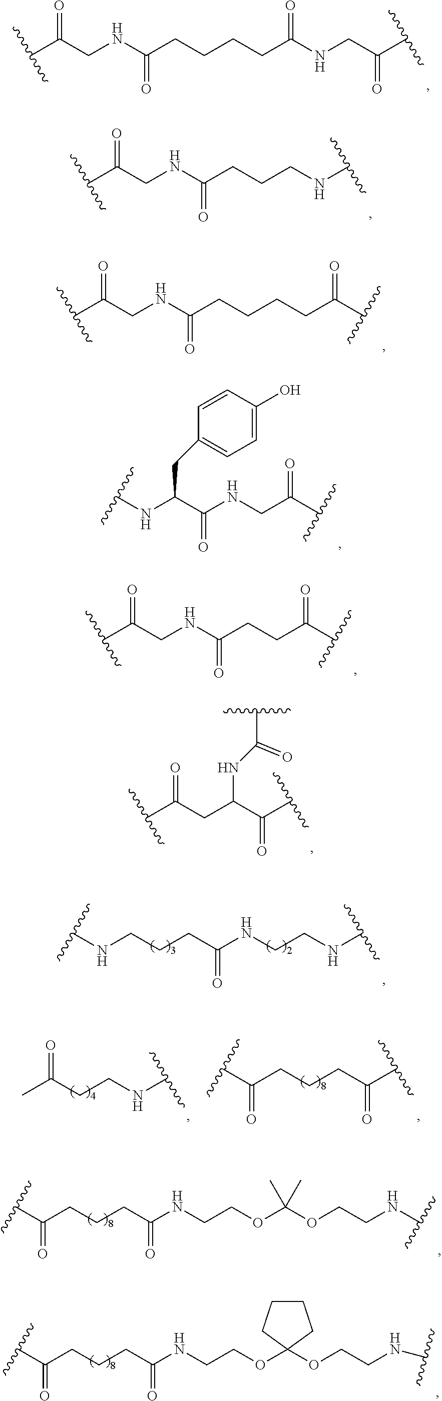

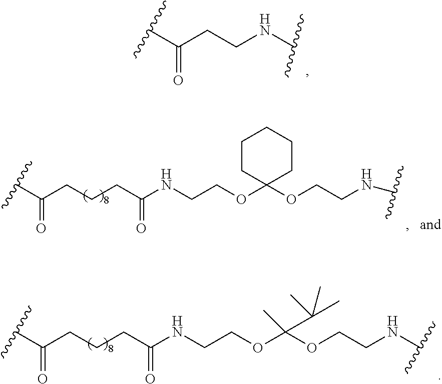

In certain embodiments, L₁, L₂, L₃, L₄, L₅, L₆, and/or L₇ independently comprise, or taken together comprise, a structure selected from among:

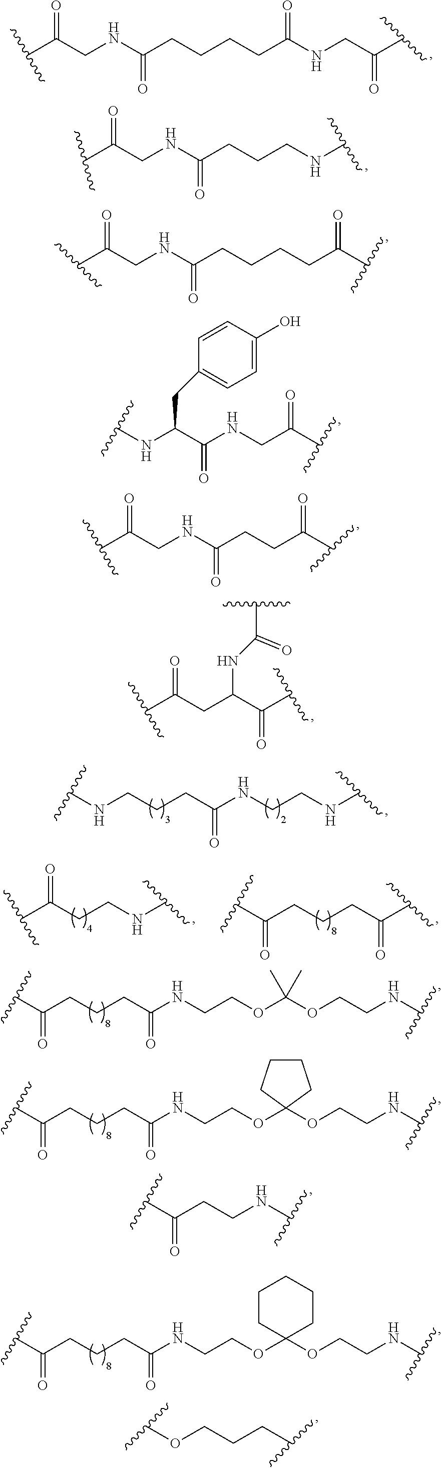

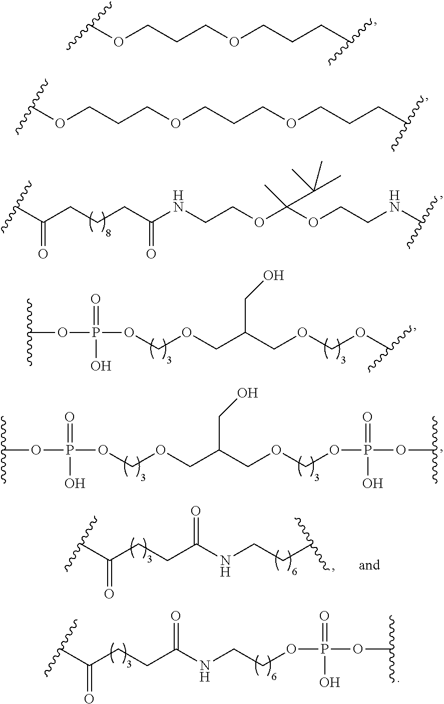

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among:

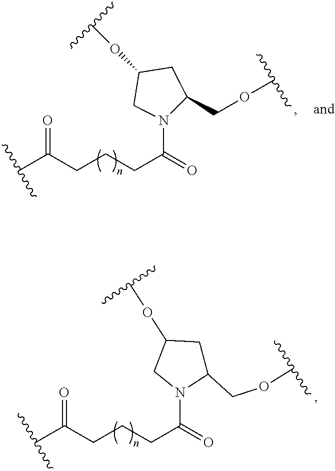

wherein n is an integer in the range from 1 to 20, inclusive.

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among;

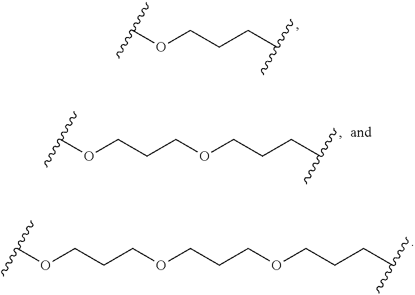 [SMILES: *CCCO* ; *CCCOCCCO* ; *CCCOCCCOCCCO*]

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among:

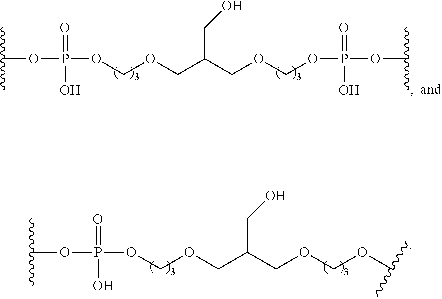

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among:

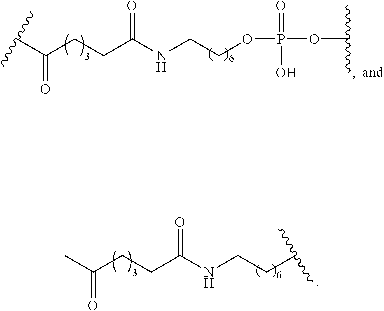

In certain embodiments, L₁, L₂, L₃, and L₄ (or L₁, L₂, L₃, L₄, and L₅, or L₁, L₂, L₃, L₄, L₅, L₆, and L₇) independently comprise or together have the structure:

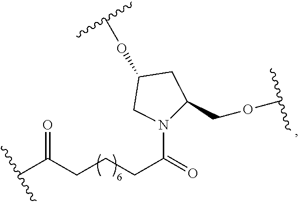

In certain embodiments, L₁, L₂, L₃, and L₄ (or L₁, L₂, L₃, L₄, and L₅, or L₁, L₂, L₃, L₄, L₅, L₆, and L₇) independently comprise or together have the structure:

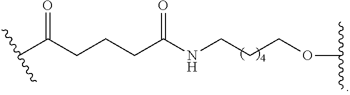

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among:

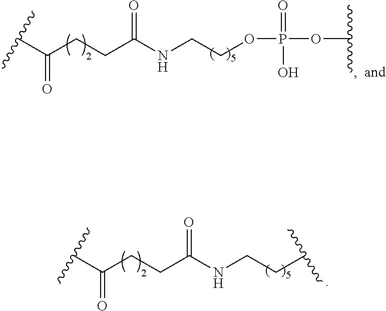

In certain embodiments, L₁, L₂, L₃, and L₄ (or L₁, L₂, L₃, L₄, and L₅, or L₁, L₂, L₃, L₄, L₅, L₆, and L₇) independently comprise or together comprise the structure selected from among:

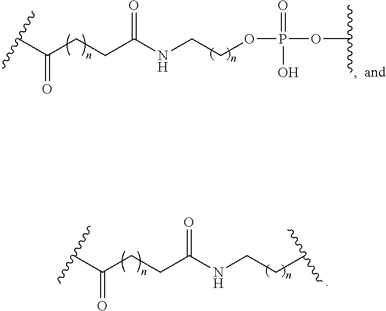

wherein each n is independently 0, 1, 2, 3, 4, 5, 6, or 7.

In some embodiments, each of L₁, L₂, L₃, and L₄ (or L₁, L₂, L₃, L₄, and L₅, or L₁, L₂, L₃, L₄, L₅, L₆, and L₇) is independently absent, a bond, an substituted or unsubstituted alkyl linker, an substituted or unsubstituted polyethylene glycol (PEG) linker, an substituted or unsubstituted heteroalkyl linker, an substituted or unsubstituted heteroaryl linker, a phosphodiester bond, or a phosphorothioate bond.

In some embodiments, L₁ is a bond.

In some embodiments, L₂ is an substituted or unsubstituted PEG linker. In some embodiments, the PEG linker is three or four PEG units in length. In certain embodiments, L₂ comprises the structure

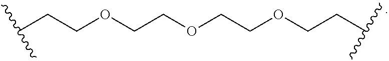 [SMILES: *CCOCCOCCOCC*]

In some embodiments, the PEG linker is two or three PEG units in length.

In some embodiments, L₃ is an substituted or unsubstituted heteroaryl linker. In some embodiments, L₃ is an substituted or unsubstituted partially unsaturated heteroaryl linker. In certain embodiments, L₃ comprises the structure

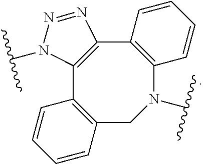 [SMILES: *N1Cc2ccccc2-c2c(nnn2*)-c2ccccc21]

In some embodiments, L₄ is an substituted or unsubstituted heteroalkyl linker. In some embodiments, the heteroalkyl linker is substituted with one or more ═O substituents. In certain embodiments, L₄ comprises the structure

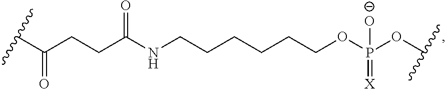

wherein X is O or S.

In some embodiments, L₁, L₂, L₃, and L₄ together comprise the structure

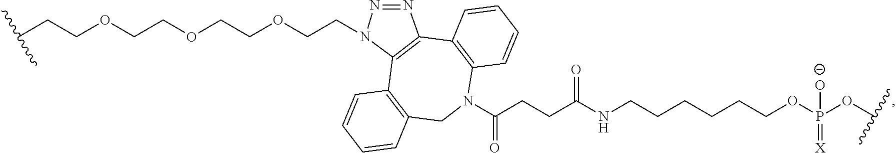

wherein X is O or S.

In some embodiments, one of L₃ and L₄ is a phosphodiester bond or a phosphorothioate bond, and the other of L₃ and L₄ is a bond. In certain embodiments, L₁, L₂, L₃, and L₄ together comprise the structure

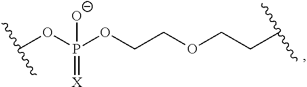

wherein X is O or S.

In certain embodiments, L₁, L₂, L₃, and L₄ together comprise the structure

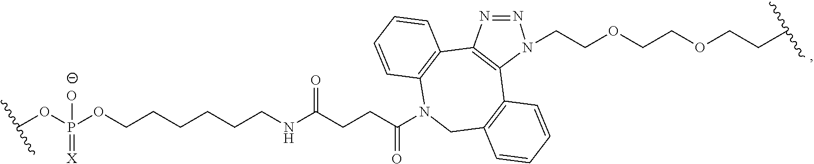

wherein X is O or S.

In some embodiments, each of L₁, L₂, L₃, L₄, and L₅ is independently absent, a bond, an substituted or unsubstituted alkyl linker, an substituted or unsubstituted polyethylene glycol (PEG) linker, an substituted or unsubstituted heteroalkyl linker, an substituted or unsubstituted heteroaryl linker, a phosphodiester bond, or a phosphorothioate bond.

In some embodiments, L₁ and L₅ are each an substituted or unsubstituted PEG linker. In some embodiments, L₁ and L₅ are each an substituted or unsubstituted PEG linker three PEG units in length.

In some embodiments, L₂ is an substituted or unsubstituted heteroalkyl linker. In some embodiments, the heteroalkyl linker is substituted with one or more ═O substituents. In certain embodiments, L₂ comprises the structure

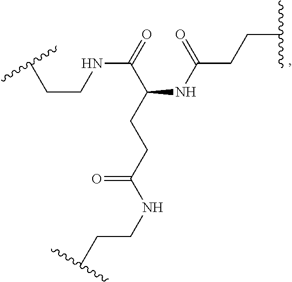 [SMILES: *CCNC(=O)CC[C@H](NC(=O)CC*)C(=O)NCC*]

In some embodiments, L₃ is an substituted or unsubstituted heteroaryl linker. In some embodiments, L₃ is an substituted or unsubstituted partially unsaturated heteroaryl linker. In certain embodiments, L₃ comprises the structure

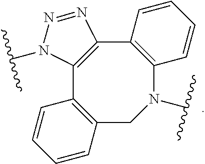 [SMILES: *N1Cc2ccccc2-c2c(nnn2*)-c2ccccc21]

In some embodiments, L₄ is an substituted or unsubstituted heteroalkyl linker. In some embodiments, the heteroalkyl linker is substituted with one or more ═O substituents. In certain embodiments, L₄ comprises the structure

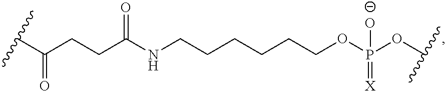

wherein X is O or S.

In some embodiments, L₁, L₂, L₃, L₄, and L₅ together comprise the structure

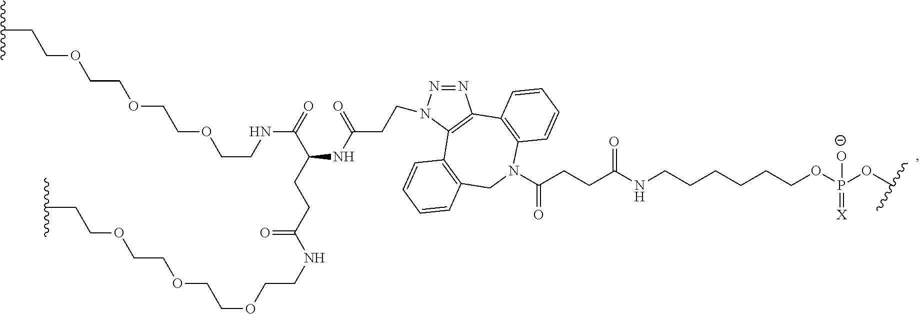

### X is O or S.

In some embodiments, each of L₁, L₂, L₃, L₄, L₅, L₆, and L₇ is independently absent, a bond, an substituted or unsubstituted alkyl linker, an substituted or unsubstituted polyethylene glycol (PEG) linker, an substituted or unsubstituted heteroalkyl linker, an substituted or unsubstituted heteroaryl linker, a phosphodiester bond, or a phosphorothioate bond.

In some embodiments, L₁ is an substituted or unsubstituted PEG linker. In certain embodiments, L₁ is an substituted or unsubstituted PEG linker two or three PEG units in length.

In some embodiments, L₂ and L₅ are each independently an substituted or unsubstituted PEG linker. In some embodiments, L₂ and L₅ are each independently an substituted or unsubstituted PEG linker three or four PEG units in length. In certain embodiments, L₁, L₂, and L₅ together comprise the structure

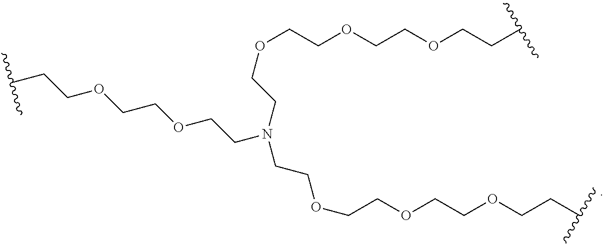 [SMILES: *CCOCCOCCOCCN(CCOCCOCC*)CCOCCOCCOCC*]

In some embodiments, L₃ and L₆ are each independently an substituted or unsubstituted heteroaryl linker. In some embodiments, L₃ and L₆ are each independently an substituted or unsubstituted partially unsaturated heteroaryl linker. In certain embodiments, L₃ and L₆ each comprise the structure

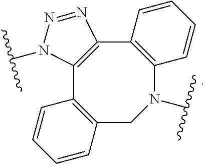 [SMILES: *N1Cc2ccccc2-c2c(nnn2*)-c2ccccc21]

In some embodiments, L₄ and L₇ are each independently an substituted or unsubstituted heteroalkyl linker. In some embodiments, the heteroalkyl linker is substituted with one or more ═O substituents. In certain embodiments, L₄ and L₇ each comprise the structure

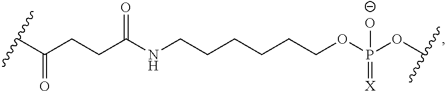

wherein X is O or S.

In some embodiments, L₁, L₂, L₃, L₄, L₅, L₆, and L₇ together comprise the structure

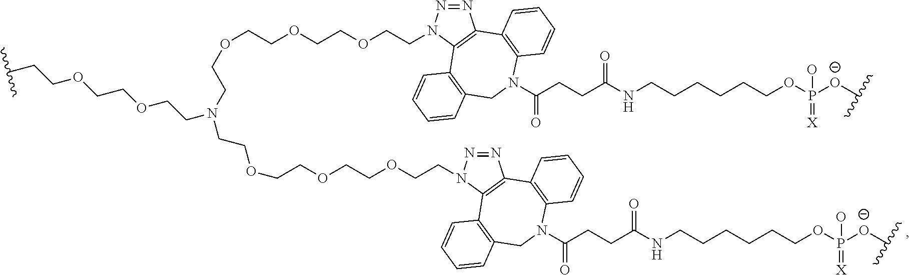

wherein X is O or S.

### Methods of Making Compounds

Compounds of the present disclosure can be made by means known in the art of organic synthesis. Methods for optimizing reaction conditions, and minimizing competing by-products, if necessary, are known in the art. Reaction optimization and scale-up may advantageously utilize high-speed parallel synthesis equipment and computer-controlled microreactors (e.g., Design And Optimization in Organic Synthesis, 2^{nd} Edition, Carlson R, Ed, 2005; Elsevier Science Ltd.; Jahnisch, K et al., Angew. Chem. Int. Ed. Engl. 2004 43: 406; and references therein). Additional reaction schemes and protocols may be determined by the skilled artisan by use of commercially available structure-searchable database software, for instance, SciFinder® (CAS division of the American Chemical Society) and CrossFire Beilstein® (Elsevier MDL), or by appropriate keyword searching using an internet search engine such as Google® or keyword databases such as the U.S. Patent and Trademark Office text database.

As can be appreciated by the skilled artisan, methods of synthesizing the compounds of the formulae herein will be evident to those of ordinary skill in the art, including in the schemes and examples herein. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired compounds of the present disclosure.

The compounds herein may also contain linkages (e.g., carbon-carbon bonds) wherein bond rotation is restricted about that particular linkage, e.g., restriction resulting from the presence of a ring or double bond. Accordingly, all cis/trans and E/Z isomers are expressly included in the present disclosure. The compounds herein may also be represented in multiple tautomeric forms; in such instances, the present disclosure expressly includes all tautomeric forms of the compounds described herein, even though only a single tautomeric form may be represented. All such isomeric forms of such compounds herein are expressly included in the present disclosure. All crystal forms and polymorphs of the compounds described herein are expressly included in the present disclosure. Also embodied are extracts and fractions comprising compounds of the present disclosure. The term “isomers” is intended to include diastereoisomers, enantiomers, regioisomers, structural isomers, rotational isomers, tautomers, and the like. For compounds which contain one or more stereogenic centers, e.g., chiral compounds, the methods of the present disclosure may be carried out with an enantiomerically enriched compound, a racemate, or a mixture of diastereomers. All isomers of compounds delineated herein are expressly included in the present disclosure.

Preferred enantiomerically enriched compounds have an enantiomeric excess of 50% or more. More preferably, the compound has an enantiomeric excess of 60%, 70%, 80%, 90%, 95%, 98%, 99%, or more. In preferred embodiments, only one enantiomer or diastereomer of a chiral compound of the present disclosure is administered to cells or a subject.

### Methods of Treatment

In one aspect, provided are methods of treating a subject suffering from or susceptible to a disorder or disease, comprising administering to the subject an effective amount of a compound or pharmaceutical composition described herein.

In other aspects, provided are methods of treating a subject suffering from or susceptible to a disorder or disease, wherein the subject has been identified as in need of modulation of the function of a protein, comprising administering to said subject in need thereof, an effective amount of a compound or pharmaceutical composition described herein, such that said subject is treated for said disorder.

In one aspect, provided are methods of delivering a therapeutic oligonucleotide to the brain of a subject, comprising contacting the subject with a compound or pharmaceutical composition described herein, in an amount and under conditions sufficient to target the brain.

In one aspect, provided are methods of modulating protein function in a subject, comprising contacting the subject with a compound of any of the formula herein (e.g., Formulae I, I′, I-VIII, II-a, and II-b), in an amount and under conditions sufficient to modulate protein function.

In one embodiment, the modulation is inhibition.

In some embodiments, provided are methods for targeting hepatic cells in a subject, comprising administering to said subject in need thereof, an effective amount of a compound, oligonucleotide, or pharmaceutical composition of any of the formula herein (e.g., Formulae I, I′, I-VIII, II-a, and II-b) in an amount and under conditions sufficient to target hepatic cells.

In certain embodiments, provided are methods of treating a disease, disorder or symptom thereof, wherein the disorder is cancer, a proliferative disease, a neurodegenerative disease, an autoimmune or inflammatory disorder, an infection, a metabolic disorder, a hematologic disorder, or a cardiovascular disease.

In certain embodiments, the disorder or disease is cancer or a proliferative disease. In certain embodiments, the cancer or proliferative disease includes a carcinoma, a leukemia, a blastoma, a lymphoma, a myeloma, or a melanoma, or a combination thereof. In certain embodiments, the disorder or disease is multiple myeloma, melanoma, breast cancer, pancreatic cancer, ovarian cancer, prostate cancer, hepatocellular cancer, renal cancer, leukemia, T-cell lymphoma, bone cancer, glioblastoma, neuroblastoma, oral squamous cell carcinoma, urothelial cancer, lung cancer, cervical cancer, colon cancer, head and neck squamous cell carcinoma, Burkitt's Lymphoma, esophageal cancer, Hodgkin's lymphoma, bladder cancer, or gastric cancer, or a combination thereof.

In certain embodiments, the disorder or disease is rheumatoid arthritis, spondylitis arthritis, psoriatic arthritis, multiple sclerosis, systemic lupus erythematosus, inflammatory bowel disease, graft versus host disease, transplant rejection, fibrotic disease, Crohn's Disease, type-1 diabetes, eczema, psoriasis, sepsis, airway hyperresponsiveness, ulcerative colitis, or a combination thereof.

In certain embodiments, wherein the disorder or disease is epilepsy, attention deficit disorder, Alzheimer's disease, Parkinson's Disease, Huntington's Disease, amyotrophic lateral sclerosis, spinal muscular atrophy, essential tremor, central nervous system trauma, multiple sclerosis, Charcot-Marie-Tooth (MCT), peripheral neuropathy, or cerebral ischemia, or a combination thereof.

In certain embodiments, the disorder or disease is an infection caused by virus, fungus, or bacteria, or a combination thereof.

In certain embodiments, the disorder or disease is metabolic syndrome, diabetes, obesity, high blood pressure, heart failure, cyst growth in autosomal dominant polycystic kidney disease (ADPKD), or a combination thereof.

In certain embodiments, the disorder or disease is cardiovascular stress, pressure overload, chronic ischemia, infarction-reperfusion injury, hypertension, atherosclerosis, peripheral artery disease, heart failure, hypertrophy, angina, arrhythmias, hypercholesterolemia, atherosclerosis, or stroke, or a combination thereof.

In certain embodiments, the disorder or disease is liver disease.

In certain embodiments, the subject is a mammal, preferably a primate or a human.

In another embodiment, provided are methods as described above, wherein the effective amount of the compound or oligonucleotide of any of the formula herein (e.g., Formulae I, I′, I-VIII, II-a, and II-b) is as described above.

In another embodiment, provided are methods as described above, wherein the compound or oligonucleotide of any of the formula herein (e.g., Formulae I, I′, I-VIII, II-a, and II-b) is administered intravenously, intramuscularly, subcutaneously, intracerebroventricularly, orally, or topically.

In other embodiments, provided are methods as described above, wherein the compound or oligonucleotide of any of the formula herein (e.g., Formulae I, I′, I-VIII, II-a, and II-b) is administered alone or in combination with one or more other therapeutics. In a further embodiment, the additional therapeutic agent is an anti-cancer agent, antifungal agent, cardiovascular agent, anti-inflammatory agent, chemotherapeutic agent, an anti-angiogenesis agent, cytotoxic agent, an anti-proliferation agent, metabolic disease agent, ophthalmologic disease agent, central nervous system (CNS) disease agent, urologic disease agent, or gastrointestinal disease agent.

Another object of the present disclosure is the use of a compound or oligonucleotide as described herein (e.g., a compound or oligonucleotide of Formulae I, I′, I-VIII, II-a, and II-b) in the manufacture of a medicament for use in the treatment of a disorder or disease. Another object of the present disclosure is the use of a compound or oligonucleotide as described herein (e.g., a compound or oligonucleotide of Formulae I, I′, I-VIII, II-a, and II-b) for use in the treatment of a disorder or disease. Another object of the present disclosure is the use of a compound or oligonucleotide as described herein (e.g., a compound or oligonucleotide of Formulae I, I′, I-VIII, II-a, and II-b) in the manufacture of an agricultural composition for use in the treatment or prevention of a disorder or disease in agricultural or agrarian settings.

In certain embodiments, provided are methods of treating a disease, disorder or symptom thereof, wherein the disease is a central nervous system (CNS) disease, disorder, or symptom thereof. In some embodiments, the disease is a neurodegenerative disease, disorder, or symptom thereof. In some embodiments, the disease is Alzheimer's disease, or a symptom thereof.

Exemplary CNS disorders include, but are not limited to, neurotoxicity and/or neurotrauma, stroke, multiple sclerosis, spinal cord injury, epilepsy, a mental disorder, a sleep condition, a movement disorder, nausea and/or emesis, amyotrophic lateral sclerosis, Alzheimer's disease, and substance abuse.

In certain embodiments, the CNS disorder is neurotoxicity and/or neurotrauma, e.g., for example, as a result of acute neuronal injury (e.g., traumatic brain injury (TBI), stroke, epilepsy) or a chronic neurodegenerative disorder (e.g., multiple sclerosis, Parkinson's disease, Huntington's disease, amyotrophic lateral sclerosis, Alzheimer's disease). In certain embodiments, the compounds of the present disclosure provide a neuroprotective effect, e.g., against an acute neuronal injury or a chronic neurodegenerative disorder.

In certain embodiments, the CNS disorder is a mental disorder, e.g., for example, depression, anxiety or anxiety-related conditions, a learning disability, or schizophrenia.

In certain embodiments, the CNS disorder is depression. “Depression” includes, but is not limited to, depressive disorders or conditions, such as, for example, major depressive disorders (e.g., unipolar depression), dysthymic disorders (e.g., chronic, mild depression), bipolar disorders (e.g., manic depression), seasonal affective disorder, and/or depression associated with substance abuse (e.g., withdrawal). The depression can be clinical or subclinical depression. The depression can be associated with or premenstrual syndrome and/or premenstrual dysphoric disorder.

In certain embodiments, the CNS disorder is anxiety. “Anxiety” includes, but is not limited to, anxiety and anxiety-related conditions, such as, for example, clinical anxiety, panic disorder, agoraphobia, generalized anxiety disorder, specific phobia, social phobia, obsessive-compulsive disorder, acute stress disorder, post-traumatic stress disorder, adjustment disorders with anxious features, anxiety disorder associated with depression, anxiety disorder due to general medical conditions, and substance-induced anxiety disorders, anxiety associated with substance abuse (e.g., withdrawal, dependence, reinstatement) and anxiety associated with nausea and/or emesis. This treatment may also be to induce or promote sleep in a subject (e.g., for example, a subject with anxiety).

In certain embodiments, the CNS disorder is a learning disorder (e.g., attention deficit disorder (ADD)).

In certain embodiments, the CNS disorder is schizophrenia.

In certain embodiments, the CNS disorder is a sleep condition. “Sleep conditions” include, but are not limited to, insomnia, narcolepsy, sleep apnea, restless legs syndrome (RLS), delayed sleep phase syndrome (DSPS), periodic limb movement disorder (PLMD), hypopnea syndrome, rapid eye movement behavior disorder (RBD), shift work sleep condition (SWSD), and sleep problems (e.g., parasomnias) such as nightmares, night terrors, sleep talking, head banging, snoring, and clenched jaw and/or grinding of teeth (bruxism).

In certain embodiments, the CNS disorder is a movement disorder, e.g., basal ganglia disorders, such as, for example, Parkinson's disease, levodopa-induced dyskinesia, Huntington's disease, Gilles de la Tourette's syndrome, tardive dyskinesia, and dystonia.

In certain embodiments, the CNS disorder is Alzheimer's disease.

In certain embodiments, the CNS disorder is amyotrophic lateral sclerosis (ALS).

In certain embodiments, the CNS disorder is nausea and/or emesis.

In certain embodiments, the CNS disorder is substance abuse disorder (SUD) (e.g., for instance, addiction to opiates, nicotine, cocaine, psychostimulants, and/or alcohol).

The term “neurological disease” (including, e.g., “neurodegenerative diseases) refers to any disease of the nervous system, including diseases that involve the central nervous system (brain, brainstem, and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle, and neuromuscular junctions. Substance abuse and mental illness (including, but not limited to, substance use disorder, bipolar disorder, eating disorders, and schizophrenia) are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

In certain embodiments, the subject is a mammal, preferably a primate or a human.

In another embodiment, provided are methods as described above, wherein the effective amount of the compounds provided herein is as described above.

In another embodiment, provided are methods as described above, wherein the compounds provided herein is administered intrathecally, intravenously, intramuscularly, subcutaneously, intracerebroventricularly, orally, or topically. In certain embodiments, the compound is administered intrathecally.

In other embodiments, provided are methods as described above, wherein the compound of any of the formulae provided herein is administered alone or in combination with one or more other therapeutics. In a further embodiment, the additional therapeutic agent is a central nervous system (CNS) disease agent.

Another object of the present disclosure is the use of a compound as described herein in the manufacture of a medicament for use in the treatment of a disorder or disease. Another object of the present disclosure is the use of a compound as described herein for use in the treatment of a disorder or disease.

### Pharmaceutical Compositions

In one aspect, provided are pharmaceutical compositions comprising any of the compounds described herein and a pharmaceutically acceptable carrier or pharmaceutically acceptable excipient.

A compound or composition, as described herein, can be administered in combination with one or more additional therapeutic agents (e.g., therapeutically and/or prophylactically active agents). The compounds or compositions can be administered in combination with additional therapeutic agents that improve their activity (e.g., activity (e.g., potency and/or efficacy) in treating a disease in a subject in need thereof, in preventing a disease in a subject in need thereof, and/or in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional therapeutic agent exhibits a synergistic effect that is absent in a pharmaceutical composition including one of the compounds described herein or the additional therapeutic agent, but not both.

The compound or composition can be administered concurrently with, prior to, or subsequent to one or more additional therapeutic agents, which may be useful as, e.g., combination therapies. Therapeutic agents include therapeutically active agents. Therapeutic agents also include prophylactically active agents. Therapeutic agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional therapeutic agent is a therapeutic agent useful for treating and/or preventing a disease (e.g., CNS disorder). Each additional therapeutic agent may be administered at a dose and/or on a time schedule determined for that therapeutic agent. The additional therapeutic agents may also be administered together with each other and/or with the compound or composition described herein in a single dose or administered separately in different doses. The particular combination to employ in a regimen will take into account compatibility of the compound described herein with the additional therapeutic agent(s) and/or the desired therapeutic and/or prophylactic effect to be achieved. In general, it is expected that the additional therapeutic agent(s) in combination be utilized at levels that do not exceed the levels at which they are utilized individually. In some embodiments, the levels utilized in combination will be lower than those utilized individually.

In one aspect, provided are kits comprising an effective amount of a compound provided herein, in unit dosage form, together with instructions for administering the compound to a subject suffering from or susceptible to a disease or disorder.

“Pharmaceutically acceptable salts” means or refers to physiologically and pharmaceutically acceptable salts of compounds, such as oligomeric compounds or oligonucleotides, i.e., salts that retain the desired biological activity of the parent compound and do not impart undesired toxicological effects thereto. As used herein, a pharmaceutically acceptable salt is any salt of a compound provided herein which retains its biological properties and which is not toxic or otherwise undesirable for pharmaceutical use. The pharmaceutically acceptable salts of the therapeutic agents disclosed herein include salts that are prepared with relatively nontoxic acids or bases, depending on the particular substituents found on the compounds or modified oligonucleotides described herein.

When compounds of the present disclosure contain relatively acidic functionalities, base addition salts can be obtained by contacting the neutral form of such compounds with a sufficient amount of the desired base, either neat or in a suitable inert solvent.

When compounds of the present disclosure contain relatively basic functionalities, acid addition salts can be obtained by contacting the neutral form of such compounds with a sufficient amount of the desired acid, either neat or in a suitable inert solvent.

Thus, the compounds of the present disclosure may exist as salts, such as with pharmaceutically acceptable acids. Such salts may be derived from a variety of organic and inorganic counter-ions well known in the art. Such salts include, but are not limited to: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia, or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like (see, for example, Berge et al., “Pharmaceutical Salts,” Journal of Pharmaceutical Science, 1977, 66, 1-19).

Pharmaceutically acceptable salts further include, by way of example only and without limitation, sodium, potassium, calcium, magnesium, ammonium, tetraalkylammonium, and the like, and when the compound contains a basic functionality, salts of non-toxic organic or inorganic acids, such as hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate, and the like. In some embodiments, the pharmaceutically acceptable salt of the compounds and modified oligonucleotides disclosed herein is a sodium or a potassium salt. In some embodiments, the pharmaceutically acceptable salt of the compounds and modified oligonucleotides disclosed herein is a sodium salt.

The neutral forms of the compounds are preferably regenerated by contacting the salt with a base or acid and isolating the parent compound in the conventional manner. The parent form of the compound may differ from the various salt forms in certain physical properties, such as solubility in polar solvents. In embodiments, compounds of the present disclosure contain both basic and acidic functionalities that allow the compounds to be converted into either base or acid addition salts. The neutral forms of the compounds may be regenerated by contacting the salt with a base or acid and isolating the parent compound in a conventional manner. The parent form of the compounds differs from the various salt forms in certain physical properties, such as solubility in polar solvents, but, unless specifically indicated, the salts disclosed herein are equivalent to the parent form of the compound for the purposes of the present disclosure.

When compounds of the present disclosure contain relatively basic functionalities, acid addition salts can be obtained by contacting the neutral form of such compounds with a sufficient amount of the desired acid, either neat or in a suitable inert solvent.

Thus, the compounds of the present disclosure may exist as salts, such as with pharmaceutically acceptable acids or bases. Such salts may be derived from a variety of organic and inorganic counter-ions well known in the art. Such salts include, but are not limited to: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia, or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Pharmaceutically acceptable salts further include, by way of example only and without limitation, sodium, potassium, calcium, magnesium, ammonium, tetraalkylammonium, and the like, and when the compound contains a basic functionality, salts of non-toxic organic or inorganic acids, such as hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate, and the like. In some embodiments, the pharmaceutically acceptable salt of the compounds and modified oligonucleotides disclosed herein is a sodium or a potassium salt. In some embodiments, the pharmaceutically acceptable salt of the compounds and modified oligonucleotides disclosed herein is a sodium salt.

The neutral forms of the compounds are preferably regenerated by contacting the salt with a base or acid and isolating the parent compound in the conventional manner. The parent form of the compound may differ from the various salt forms in certain physical properties, such as solubility in polar solvents. In embodiments, compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

In addition to salt forms, the present disclosure provides compounds which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Certain compounds of the present disclosure can exist in unsolvated forms as well as solvated forms, including hydrated forms. In general, the solvated forms are equivalent to unsolvated forms and are intended to be encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

The present disclosure also provides a pharmaceutical composition, comprising an effective amount of a compound described herein and a pharmaceutically acceptable excipient. In an embodiment, a compound of any of the formulae provided herein is administered to a subject using a pharmaceutically-acceptable formulation, e.g., a pharmaceutically-acceptable formulation that provides sustained delivery of the compound to a subject for at least 12 hours, 24 hours, 36 hours, 48 hours, one week, two weeks, three weeks, or four weeks after the pharmaceutically-acceptable formulation is administered to the subject.

Actual dosage levels and time course of administration of the active ingredients in the pharmaceutical compositions of the disclosure may be varied so as to obtain an amount of the active ingredient which is effective to achieve the desired therapeutic response for a particular subject, composition, and mode of administration, while being acceptably tolerant to the subject.

In use, at least one compound according to the present disclosure is administered in a pharmaceutically effective amount to a subject in need thereof in a pharmaceutical carrier by intravenous, intrathecal, intramuscular, subcutaneous, or intracerebroventricular injection or by oral administration or topical application. In accordance with the present disclosure, a compound of the disclosure may be administered alone or in conjunction with a second, different therapeutic. By “in conjunction with” is meant together, substantially simultaneously, or sequentially. In one embodiment, a compound of the disclosure is administered acutely. The compound of the disclosure may therefore be administered for a short course of treatment, such as for about 1 day to about 1 week. In another embodiment, the compound of the disclosure may be administered over a longer period of time to ameliorate chronic disorders, such as, for example, for about one week to several months depending upon the condition to be treated.

By “pharmaceutically effective amount,” as used herein, is meant an amount of a compound of the disclosure, high enough to significantly positively modify the condition to be treated but low enough to avoid serious side effects (at a reasonable benefit/risk ratio), within the scope of sound medical judgment. A pharmaceutically effective amount of a compound of the disclosure will vary with the particular goal to be achieved, the age and physical condition of the patient being treated, the severity of the underlying disease, the duration of treatment, the nature of concurrent therapy and the specific compound employed. For example, a therapeutically effective amount of a compound of the disclosure administered to a child or a neonate will be reduced proportionately in accordance with sound medical judgment. The effective amount of a compound of the disclosure will thus be the minimum amount which will provide the desired effect.

A decided practical advantage of the present disclosure is that the compound may be administered in a convenient manner such as by intrathecal, intravenous, intramuscular, subcutaneous, oral, or intra-cerebroventricular injection routes or by topical application, such as in creams or gels. Depending on the route of administration, the active ingredients which comprise a compound of the disclosure may be required to be coated in a material to protect the compound from the action of enzymes, acids and other natural conditions which may inactivate the compound. In order to administer a compound of the disclosure by a mode other than parenteral administration, the compound can be coated by, or administered with, a material to prevent inactivation.

The compound may be administered parenterally or intraperitoneally. Dispersions can also be prepared, for example, in glycerol, liquid polyethylene glycols, and mixtures thereof, and in oils.

Some examples of substances which can serve as pharmaceutical excipients, or pharmaceutical carriers (which terms are used interchangeably herein), are sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil, and oil of theobroma; polyols such as propylene glycol, glycerine, sorbitol, mannitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, antioxidants, and preservatives, can also be present. Solubilizing agents, including for example, cremaphore, and beta-cyclodextrins, can also be used in the pharmaceutical compositions herein.

Pharmaceutical compositions comprising the active compounds of the present disclosure (or prodrugs thereof) can be manufactured by means of conventional mixing, dissolving, granulating, dragee-making levigating, emulsifying, encapsulating, entrapping, or lyophilization processes. The compositions can be formulated in conventional manner using one or more physiologically acceptable carriers, diluents, excipients, or auxiliaries, which facilitate processing of the active compounds into preparations that can be used pharmaceutically. The compositions herein can be made by combining (e.g., contacting, mixing, dissolving, granulating, dragee-making, levigating, emulsifying, encapsulating, entrapping, or lyophilizing) a compound delineated herein with one or more suitable carriers, diluents, excipients, or auxiliaries, including those described herein (e.g., for pharmaceutical, agricultural, or veterinary use).

Pharmaceutical compositions of the present disclosure can take a form suitable for virtually any mode of administration, including, for example, intrathecal, topical, ocular, oral, buccal, systemic, nasal, injection, transdermal, rectal, vaginal, and the like, or a form suitable for administration by inhalation or insufflation.

Systemic formulations include those designed for administration by injection, e.g., subcutaneous, intravenous, intramuscular, intrathecal, or intraperitoneal injection, as well as those designed for transdermal, transmucosal, oral, or pulmonary administration.

Useful injectable preparations include sterile suspensions, solutions, or emulsions of the active compound(s) in aqueous or oily vehicles. The compositions also can contain formulating agents, such as suspending, stabilizing and/or dispersing agent. The formulations for injection can be presented in unit dosage form (e.g., in ampules or in multidose containers) and can contain added preservatives.

Alternatively, the injectable formulation can be provided in powder form for reconstitution with a suitable vehicle, including but not limited to, sterile pyrogen free water, buffer, dextrose solution, and the like, before use. To this end, the active compound(s) can be dried by any art-known technique, such as lyophilization, and reconstituted prior to use.

For prolonged delivery, the active compound(s), or prodrug(s) can be formulated as a depot preparation for administration by implantation or intramuscular injection. The active ingredient can be formulated with suitable polymeric or hydrophobic materials (e.g., as an emulsion in an acceptable oil) or ion exchange resins, or as sparingly soluble derivatives, e.g., as a sparingly soluble salt.

Alternatively, other pharmaceutical delivery systems can be employed. Liposomes and emulsions are well-known examples of delivery vehicles that can be used to deliver active compound(s), oligonucleotide(s), or prodrug(s). Certain organic solvents such as dimethylsulfoxide (DMSO) also can be employed.

The pharmaceutical compositions can, if desired, be presented in a pack or dispenser device that can contain one or more unit dosage forms containing the active compound(s). The pack can, for example, comprise metal or plastic foil, such as a blister pack. The pack or dispenser device can be accompanied by instructions for administration.

The active compound(s), or prodrug(s) of the present disclosure, or compositions thereof, will generally be used in an amount effective to achieve the intended result, for example in an amount effective to treat or prevent the particular disease being treated. The compound(s) and oligonucleotide(s) can be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient can still be afflicted with the underlying disorder. Therapeutic benefit also includes halting or slowing the progression of the disease, regardless of whether improvement is realized.

For prophylactic administration, the compound can be administered to a patient at risk of developing one of the previously described diseases. A patient at risk of developing a disease can be a patient having characteristics placing the patient in a designated group of at-risk patients, as defined by an appropriate medical professional or group. A patient at risk may also be a patient that is commonly or routinely in a setting where development of the underlying disease could occur. In other words, an at-risk patient is one who is commonly or routinely exposed to the disease or illness causing conditions or may be acutely exposed for a limited time. Alternatively, prophylactic administration can be applied to avoid the onset of symptoms in a patient diagnosed with the underlying disorder.

The amount of compound administered will depend upon a variety of factors, including, for example, the particular indication being treated, the mode of administration, whether the desired benefit is prophylactic or therapeutic, the severity of the indication being treated, the age and weight of the patient, the bioavailability of the particular active compound, and the like. Determination of an effective dosage is well within the capabilities of those skilled in the art.

Effective dosages can be estimated initially from in vitro assays. For example, an initial dosage for use in animals can be formulated to achieve a circulating blood or serum concentration of active compound that is at or above an IC₅₀ of the particular compound as measured in an in vitro assay, such as an in vitro fungal MIC or MFC, and other in vitro assays. Calculating dosages to achieve such circulating blood or serum concentrations taking into account the bioavailability of the particular compound is well within the capabilities of skilled artisans. For guidance, see “General Principles,” In: Goodman and Gilman's The Pharmaceutical Basis of Therapeutics, Chapter 1, pp. 1-112, 13th ed., McGraw-Hill, and the references cited therein, which are incorporated herein by reference.

Initial dosages also can be estimated from in vivo data, such as animal models. Animal models useful for testing the efficacy of compounds to treat or prevent the various diseases described above are well-known in the art.

Dosage amounts will typically be in the range of from about 0.0001 or 0.001 or 0.01 mg/kg/day to about 100 mg/kg/day, but can be higher or lower, depending upon, among other factors, the activity of the compound, its bioavailability, the mode of administration, and various factors discussed above. Dosage amount and interval can be adjusted individually to provide plasma levels of the compound(s) that are sufficient to maintain therapeutic or prophylactic effect. In cases of local administration or selective uptake, such as local topical administration, the effective local concentration of active compound(s) cannot be related to plasma concentration. Skilled artisans will be able to optimize effective local dosages without undue experimentation.

Preferably, the compound(s) will provide therapeutic or prophylactic benefit and will have acceptable tolerability. Tolerability of the compound(s) and oligonucleotide(s) can be determined using standard pharmaceutical procedures. The dose ratio between non-tolerable and therapeutic (or prophylactic) effect is the therapeutic index. Compounds(s) that exhibit high therapeutic indices are preferred.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### EXAMPLES

In order that the embodiments described herein may be more fully understood, the following examples are set forth. The examples described in this application are offered to illustrate the compounds, compositions, and methods provided herein and are not to be construed in any way as limiting their scope. The following examples and related sequence listing accompanying this filing may identify sequence as either “RNA” or “DNA”; however, as disclosed herein, those sequences may be modified with any combination of chemical modifications. One of skill in the art will readily appreciate that the designation of a sequence as “RNA” or “DNA” is, in certain instances, arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (methylated uracil for natural uracil of RNA). Accordingly, nucleic acid sequences provided herein, including, but not limited to, those in the sequence listing, are intended to encompass nucleic acids containing any combination of natural or modified RNA and/or DNA, including, but not limited to, such nucleic acids having modified nucleobases.

### General Experimental Procedures

Definitions of variables in the structures in schemes herein are commensurate with those of corresponding positions in the formulae delineated herein.

### Common Abbreviations

A Adenine
ACN acetonitrile
br broad
C Cytosine
CDI carbonyldiimidazole
d doublet
DCM dichloromethane
dd doublet of doublets
dba dibenzylideneacetone
DBCO Azadibenzocyclooctyne
DFAA difluoroacetic anhydride
DIPEA or DIEA diisopropylethylamine
DMF dimethylformamide
DMSO dimethyl sulfoxide
DMT or DMTr Dimethoxytrityl
dppf 1,1′-ferrocenediyl-bis(diphenylphosphine)
EDCI 1-ethyl-3-(3-dimethylaminopropyl)carbodiimide
EtOAc ethyl acetate
G Guanine
h hour(s)
I2O diiodine oxide
HRMS high resolution mass spectrometry
HPLC high performance liquid chromatography
LCMS liquid chromatography and mass spectrometry
MPLC medium pressure liquid chromatography
MSCl methane sulfonyl chloride
MS mass spectrometry
MW microwave
m multiplet
MeOH methanol
min minutes
mL milliliter(s)
MWCO molecular weight cut off
m/z mass to charge ratio
NB new backbone
NMP N-methyl-2-pyrrolidone
NMR nuclear magnetic resonance
ppm parts per million
Py pyridine
Rf retention factors
rt or RT room temperature
Rₜ retention time
s singlet
t triplet
U Uracil
TBD triazabicyclodecene
TBS or TBDMS tertiary butyl dimethylsilyl
TEA triethylamine
TFAA trifluoroacetic anhydride
TLC thin layer chromatography

### Example 1: General Synthetic Procedures

The NB strands were synthesized on solid phase by using an oligonucleotide synthesizer Oligopilot100 (Cytiva Life Sciences). Solid support (CPG, 80-90 μmol/g, 500 A) was purchased from LGC-Biosearch Technologies, Petaluma, CA, and loaded to 150-300 μmol scales. All RNA and 2′ modified RNA phosphoramidites were purchased from Hongene Biotech (Union City, CA). Specifically the 2′-O-methyl phosphoramidites contained 5′-O-(4,4′-Dimethoxytrityl)-N⁶-benzoyl-2′-O-methyl-adenosine-3′-O-[(2-cyanoethyl)-(N,N-diisopropyl)]-phosphoramidite, 5′-O-(4,4′-Dimethoxytrityl)-N⁴-acetyl-2′-O-methyl-cytidine-3′-O-[(2-cyanoethyl)-(N,N-diisopropyl)]-phosphoramidite, 5′-O-(4,4′-Dimethoxytrityl)-N²-isobutyryl-2′-O-methyl-guanosine-3′-O-[(2-cyanoethyl)-(N,N-diisopropyl)]-phosphoramidite, and 5′-O-(4,4′-Dimethoxytrityl)-2′-O-methyl-uridine-3′-O-[(2-cyanoethyl)-(N,N-diisopropyl)]-phosphoramidite. As well as the 2′-Fluoro contained 5′-O-(4,4′-Dimethoxytrityl)-N⁶-benzoyl-2′-fluoroadenosine-3′-O-[(2-cyanoethyl)-(N,N-diisopropyl)]-phosphoramidite, 5′-O-(4,4′-Dimethoxytrityl)-N⁴-acetyl-2′-fluorocytidine-3′-O-[(2-cyanoethyl)-(N,N-diisopropyl)]-phosphoramidite, 5′-O-(4,4′-Dimethoxytrityl)-N²-isobutyryl-2′-fluoroguanosine-3′-O-[(2-cyanoethyl)-(N,N-diisopropyl)]-phosphoramidite, and 5′-O-(4,4′-Dimethoxytrityl)-2′-fluorouridine-3′-O-[(2-cyanoethyl)-(N,N-diisopropyl)]-phosphoramidite. In order to create phosphorohioate linkages a 0.1M solution of 3-((Dimethylamino-methylidene)amino)-3H-1,2,4-dithiazole-3-thione (DDTT obtained from Chemgenes, Wilmington, MA) was used for 4-6 minutes and to create the phosphodiester linkage a solution of 0.05M I20 in Pyridine/Water (Sigma Aldrich, St Louis, MO) was used. Following the oxidation/sulfurization a mixture of 20% n-Methylimidazole in Acetonitrile, and 40% Acetic Anhydride in 60% Lutidine in Acetonitrile (Sigma Aldrich, St Louis, MO) were used to acetylate any unreacted chain attached to the CPG.

Phosphoramidites were dissolved in anhydrous acetonitrile (0.2M) and molecular sieves (4A) were added and set overnight (Sigma Aldrich, St. Louis, MO). For the oligonucleotide chain used 5-(Ethylthio)-1H-Tetrazole (ETT, 0.6 in acetonitrile, from Sigma Aldrich) as activator solution. The NB modification (0.15M, provided in house) of the strand was dissolved in Dichloromethane:Acetonitrile (3:1) and used 5-(Ethylthio)-1H-Tetrazole solution (ETT, 0.6M in acetonitrile, from Sigma Aldrich) for the reaction to proceed to completion. Coupling times were 60 minutes (NB) and 6 minutes (2′-O-Me/2′-Fluoro) carried out at 3.0 equivalents for each step. Prior to coupling the support bound oligonucleotide is treated with a solution of Dichloroacetic Acid in Dichloromethane (3% Deblock, Sigma Aldrich) and washed with Anhydrous Acetonitrile.

### Cleavage and Deprotection of Support Bound Oligomer.

After finalization of the solid phase synthesis the support was treated with AMA solution, a 1:1 volume solution of NH₄OH:CH₃NH₂ (Fisher Scientific, Spectrum Chemicals), for 20 minutes at 65° C. The solution was then evaporated.

Before proceeding to purification, in-process analysis is performed on analytical HPLC and LCMS to record the rough crude purity and to identify the target mass of the oligonucleotide and monitor the completion of deprotection by LCMS.

### LCMS Method

LCMS condition: The column used is a Waters XBridge Oligonucleotide BEH C18 Column, 130 Å, 2.5 μm, 2.1 mm×50 mm (P/N 186003952). Buffer solutions are 400 mM HFIP+15 mM TEA (buffer A) and 100% methanol (buffer B). Gradient was set at 5-50% Buffer B over 2 minutes at 70° C. with a flowrate of 0.5 mL/minute.

### Concentration by TFF

The crude oligos are then concentrated using Pall Minimate EVO System (Product ID: OAPMPUNV). Cassette used is the Pall Minimate TFF capsule with 3 k Omega membrane.

### Purification

Purification was performed using reverse phase HPLC. The column used is a Phenomenex Clarity 5 μm Oligo-RP AXIOS, 250×30 mm (P/N: OOG-4442-UO-AX). Buffer solution mixtures are 100 mM TEAA, 5% ACN at pH of 7.0 (buffer A) and 1:1 acetonitrile:methanol (buffer B). Gradient was set at 5-30% Buffer B over 60 minutes at 60° C. with a flowrate of 20 mL/minute.

After purification, fractions are analyzed with reverse phase UPLC. The column used is a Waters ACQUITY UPLC Oligonucleotide BEH C18 1.7 μm, 2.1×50 mm (P/N: 186003949). Buffer solution mixtures are 100 mM TEAA, 5% ACN at pH of 7.0 (buffer A) and 1:1 acetonitrile:methanol (buffer B). Gradient was set at 5-30% Buffer B over 5 minutes at 80° C. with a flowrate of 1.0 mL/minute. The minimum spec of the purified pool is 85%.

### Desalting

Once a pool has been established, the oligos are then desalted using Pall Minimate EVO System (Product ID: OAPMPUNV). Cassette used is the Pall Minimate TFF capsule with 3 k Omega membrane (Product ID: OA003C12). Retentate is collected for lyophilization or annealing directly. Off-white powder was obtained after lyophilization.

### Example 2: Synthesis of dinucleotide 18

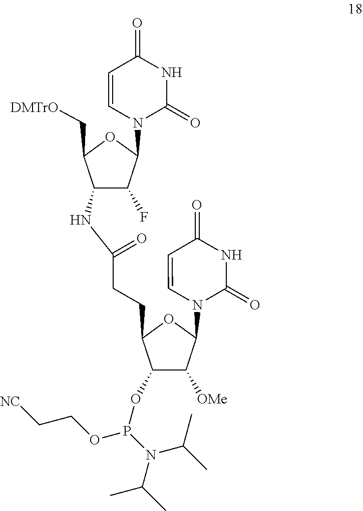

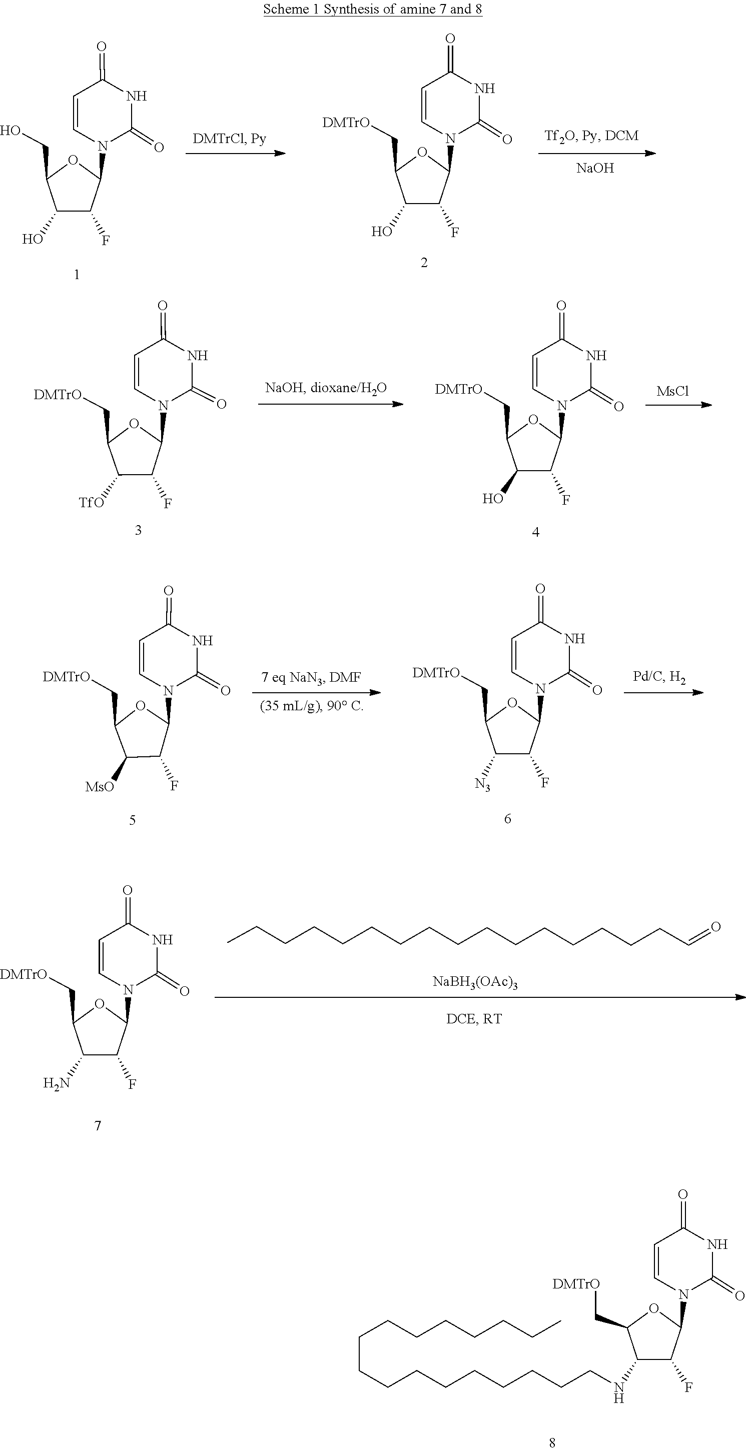

### Synthesis of 2

A solution of 1-((2R,3R,4R,5R)-3-fluoro-4-hydroxy-5-(hydroxymethyl) tetrahydrofuran-2-yl)pyrimidine-2,4(1H,3H)-dione 1 (5 g, 20.309 mmol, 1 eq) in Pyridine (50 mL) was treated with DMTr-Cl (6.54 g, 21.324 mmol, 1.05 eq) for 2 h at room temperature under nitrogen atmosphere. The resulting mixture was concentrated under reduced pressure. The crude product was purified by reverse phase flash chromatography using CH₃CN/H₂O to afford DMT ether 2 (9.5 g, 85.27%) as a light-yellow powder.

### Synthesis of 3

A solution 2 (20 g, 36.459 mmol, 1 eq) in DCM (680 mL) was treated with Pyridine (10.09 g, 127.607 mmol, 3.5 eq) for 5 min at 0° C. under nitrogen atmosphere followed by the addition of Tf₂O (17.00 g, 60.256 mmol, 1.65 eq) dropwise at 0° C. The resulting mixture was extracted with CH₂Cl₂ three times. The combined organic layers were washed with saturated CuSO₄ aqueous solution, dried over anhydrous Na₂SO₄. After filtration, the filtrate was concentrated under reduced pressure. The resulting mixture was concentrated under reduced pressure. The crude product 3 was used in the next step directly without further purification.

### Synthesis of 4

To a solution of triflate 3 (20.8 g, 30.560 mmol, 1 eq) in dioxane (450 mL) were added NaOH (17.11 mL, 171.136 mmol, 10 N, 5.60 eq) and water (83.86 mL, 4654.899 mmol, 152.32 eq). Reaction mixture was stirred overnight at room temperature under nitrogen atmosphere. The resulting mixture was extracted with EA. The combined organic layers were washed with brine, dried over anhydrous Na₂SO₄. After filtration, the filtrate was concentrated under reduced pressure. The residue was purified by silica gel column chromatography, eluted with DCM/MeOH/EA to afford product 4 (10.0 g, purity: 90%, two steps: 45%) as a light-yellow foam.

### Synthesis of 5

To a solution of 4 (2.0 g, 3.646 mmol, 1 eq) in Pyridine (33 mL) was added MsCl (1.67 g, 14.584 mmol, 4 eq) at 0° C. under nitrogen atmosphere. The reaction was stirred overnight. The resulting mixture was poured into the saturated NaHCO₃ solution and extracted with CHCl₃. The combined organic layers were washed with CuSO₄ and brine, dried over anhydrous Na₂SO₄. After filtration, the filtrate was concentrated under reduced pressure. The residue was purified by silica gel column chromatography, eluted with DCM/MeOH (50:1) to afford compound 5 (1.95 g, 85%) as a light yellow foam.

### Synthesis of 6

To a solution of 5 (2 g, 3.192 mmol, 1 eq) in DMF (68 ml, 869.373 mmol, 272.36 eq) was added NaN₃ (1.45 g, 22.344 mmol, 7 eq). The mixture was stirred for 2 days at 90° C. under nitrogen atmosphere. Then, the resulting mixture was poured into NaHCO₃ solution and extracted with DCM. The combined organic layers were washed with brine, dried over anhydrous Na₂SO₄. After filtration, the filtrate was concentrated under reduced pressure. The residue was purified by silica gel column chromatography, eluted with DCM/MeOH (200:1) to afford azide 6 (1.2 g, purity: 67%) as a light-yellow oil.

### Synthesis of 7

To a solution of azide 6 (5.65 g, 67% purity, 1 eq) in 56 ml MeOH was added Pd/C (10%, 0.57 g) under nitrogen atmosphere in a 250 mL round-bottom flask. The mixture was stirred at room temperature for 5 h under hydrogen atmosphere using a hydrogen balloon, filtered through a Celite pad, and concentrated under reduced pressure. The crude product was purified by reverse phase flash chromatography with CH₃CN/H₂O to afford amine 7 (2.74 g, 51%) as a white solid.

### Synthesis of 8

A mixture of aldehyde (0.464 g, 1.828 mmol, 1 eq), Amine (1 g, 1.828 mmol, 1 eq) and Et₃N (0.76 mL, 5.48 mmol, 3 eq) in DCE/MeOH (20/5 mL) was stirred for 30 min at RT. Sodiumtriacetoxyborohydride (581 mg, 2.74 mmol, 1.5 eq) was added and the mixture was stirred for 48 h at room temperature. LCMS showed 50-60% conversion. Reaction mixture was quenched with Aq. Saturated sodium bicarbonate 100 mL, extracted with DCM, 200 mL concentrated, and the residue was purified by column chromatography using 0-10% MeOH/DCM, to obtain inseparable mixture of product with starting material 1.16 g 80% as a white solid used as it is for next step. NMR and LCMS m/z 787 (M+1) are corresponding with product.

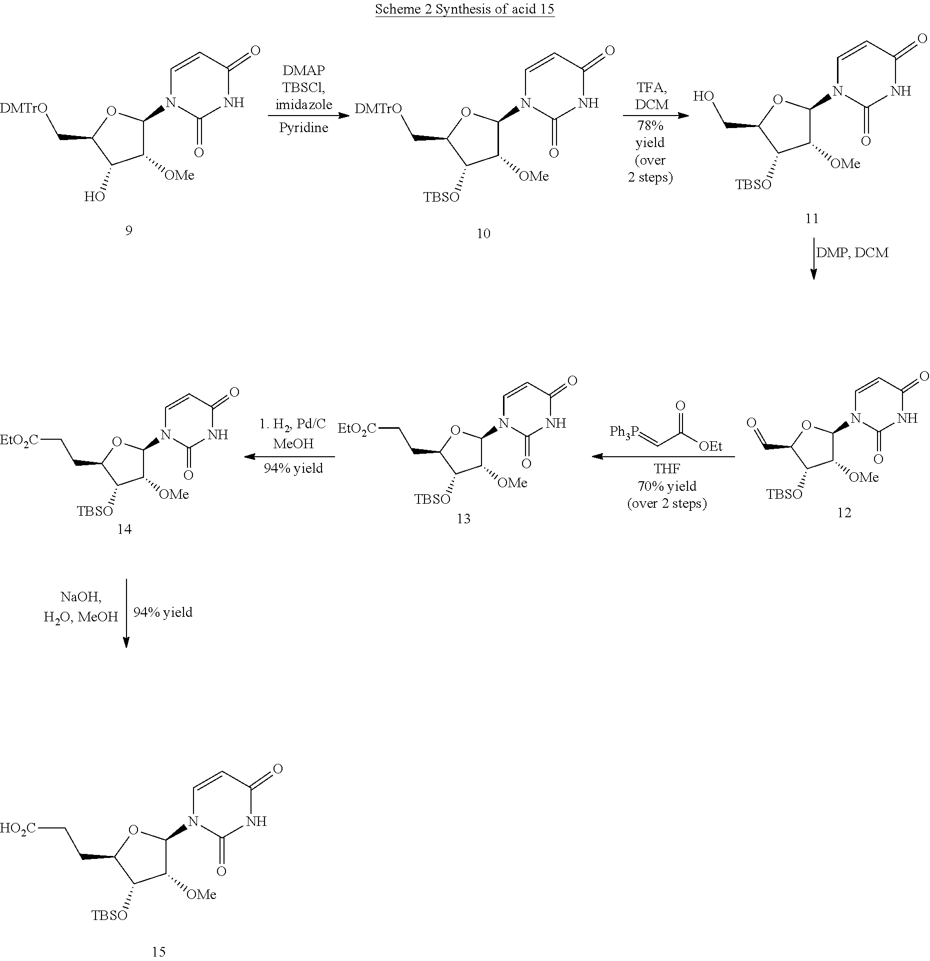

### Synthesis of 10

Imidazole (6.06 g, 89 mmol, 2.5 eq) and TBSCl (8.07 g, 54 mmol, 1.5 eq) were added as solids consecutively to a solution of alcohol 9 (20.0 g, 35.7 mmol, 1 eq) in anhydrous pyridine (200 mL) at 0° C. After 21 h, 14% SM remains, add additional TBSCl (1.77 g, 11.8 mmol, 0.33 eq) at 0° C., then stir at room temperature. After 4 h, the reaction mixture was concentrated under reduced pressure at 40° C. The resulting syrup was poured into stirring water (200 mL) and then extracted with ethyl acetate (150 mL). The aqueous layer was extracted with ethyl acetate (2×100 mL). The combined organic extracts were washed with sat'd NaCl (100 mL), dried over anhydrous sodium sulfate, filtered, and concentrated under reduced pressure to afford crude TBS ether 10 (24 g) that was used without further purification.

### Synthesis of 11

TFA (10 mL, 124 mmol, 7 eq) was added dropwise to a solution of crude DMT ether 10 (12 g, 17.9 mmol, 1 eq) in DCM (100 mL) at 0° C. The reaction mixture was then allowed to stir at room temperature. After 1 h, the reaction mixture was poured into stirring sat'd NaHCO₃ (300 mL). The aqueous layer was extracted with DCM (2×100 mL). The combined organic extracts were dried over anhydrous sodium sulfate, filtered, and concentrated under reduced pressure. The resulting residue was purified by FCC on silica gel (0460% ethyl acetate in hexanes) to afford alcohol 11 as a white solid (5.33 g). This procedure was repeated on the remaining 12 g of crude DMT ether 10, which after combination afforded alcohol 11 (10.4 g, 78% yield over two steps).

### Synthesis of 12

Sonicate to dissolve alcohol 11 (1.5 g, 4.03 mmol, 1 eq) in anhydrous DCM (40 mL). Then, Dess-Martin periodinane (2.14 g, 5.03 mmol, 1.25 eq) was added as a solid in one portion, resulting in a pink/salmon colored suspension. After 3.5 h, the reaction mixture was poured into stirring sat'd sodium thiosulfate (150 mL) and extracted with DCM (60 mL). The organic layer was washed with sat'd sodium bicarbonate (150 mL) and with sat'd NaCl (150 mL). The aqueous layers were separately extracted with DCM (2×100 mL). The combined organic extracts were dried over anhydrous sodium sulfate, filtered, and concentrated under reduced pressure to afford crude aldehyde 12 (1.57 g) as a white solid, which was used without further purification.

### Synthesis of 13

Triphenylcarbethoxymethylenephosphorane (1.76 g, 5.06 mmol, 1.25 eq) was added as a solid in one portion to a suspension of crude aldehyde 12 (1.5 g, 4.05 mmol, 1 eq) in anhydrous THF (40 mL) at room temperature. After 16 h, the reaction mixture was concentrated under reduced pressure at 30° C. to remove THF. The resulting residue was extracted with ethyl acetate (100 mL) and washed with water (50 mL) and with sat'd NaCl (50 mL). The aqueous layer was extracted with EtOAc (50 mL). The combined organic extracts were dried over anhydrous sodium sulfate, filtered, and concentrated under reduced pressure. The resulting residue was purified by FCC on silica gel (0450% ethyl acetate/hexanes) to afford ester 13 (1.24 g, 70% yield over 2 steps, 20:1 E/Z ratio. by UV).

### Synthesis of 14

A solution of alkene 13 (1.24 g, 2.81 mmol, 1 eq) in methanol (20 mL) was evacuated and backfilled with nitrogen, and charged with palladium/carbon (300 mg, 0.28 mmol, 0.1 eq). The reaction mixture was then evacuated and backfilled with hydrogen gas from a balloon. After stirring for 75 min, the reaction mixture was filtered through Celite and rinsed with methanol. The filtrate was concentrated under reduced pressure to afford ester 14 (1.17 g, 94% yield) as a white foam, which was used without further purification.

### Synthesis of 15

A solution of sodium hydroxide (528 mg, 13.2 mmol, 5 eq) in water (1 mL) was added dropwise to a solution of crude ester 14 (1.17 g, 2.64 mmol, 1 eq) in methanol (10 mL) at room temperature. After 1.5 h, the reaction mixture was concentrated under reduced pressure. The resulting residue was diluted with water (˜20 mL) resulting in a white precipitate. The reaction mixture was cooled to 0° C. with stirring, then acidified by the dropwise addition of 1 N HCl (14 mL), resulting in pH=3-4. The reaction mixture was then extracted with DCM (3×50 mL). The combined organic extracts were dried over anhydrous sodium sulfate, filtered, and concentrated under reduced pressure to afford carboxylic acid 15 (1.03 g, 94% yield) as a white foam.

### Example 3. Synthesis of dinucleotide NB-101, NB-105, NB-106, and NB-107

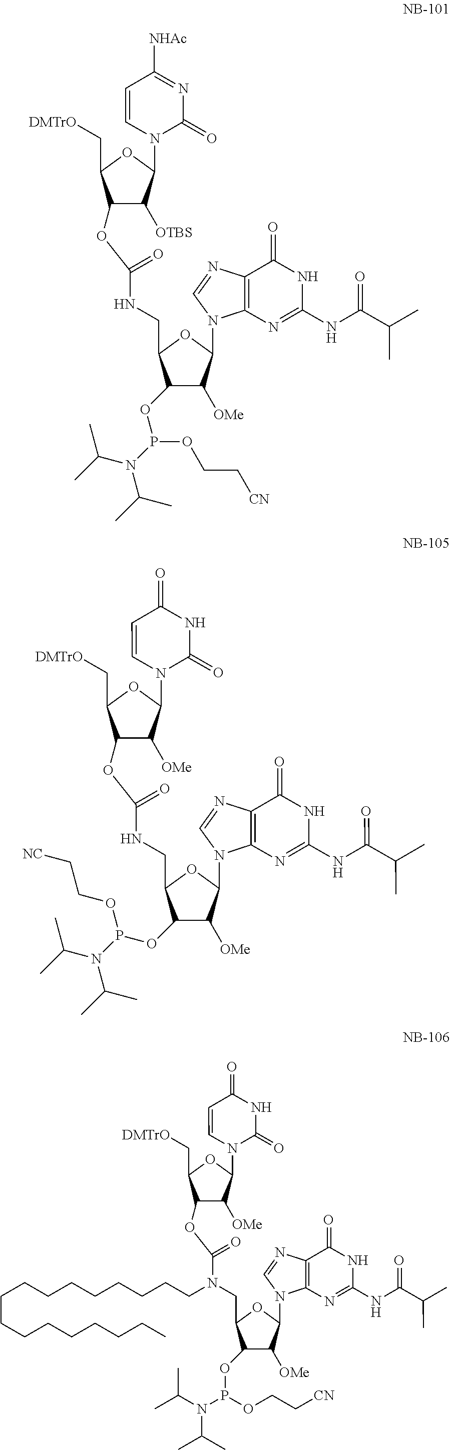

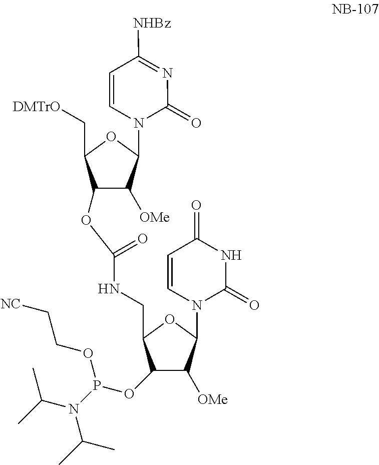

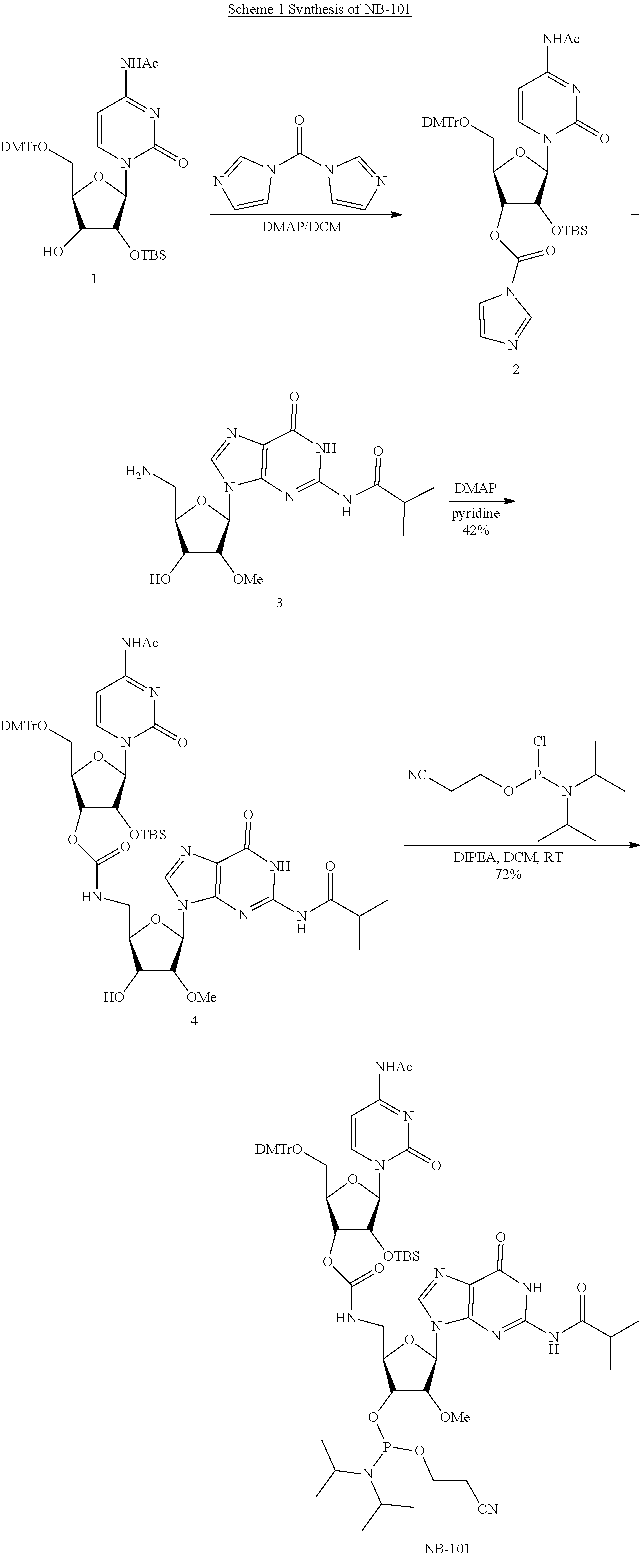

Alcohol 1 (3.0 g, 4.274 mmol, 1 eq), 1,1′ carbonyldiimidazole (1.386 g, 8.548 mmol, 2 eq) and DMAP (1.044 g, 8.548 mmol, 2 eq) were dissolved in dichloromethane (50 mL). After stirring for 14 h at room temperature, the reaction mixture was washed with saturated NH₄Cl, and extracted with DCM(2×). The combined organic layers were dried with Na₂SO₄ and concentrated to give 3.5 g crude product 2 as a white solid which was used in the next step without purification.

Compound 2 (1.2 g, 1.508 mmol, 1 eq) and amine 3 (0.663 g, 1.809 mmol, 1.2 eq) were dissolved in anhydrous pyridine (20 mL) and evaporated to dryness. The residue was redissolved in anhydrous pyridine (15 mL). DMAP (184 mg, 1.508 mmol, 1 eq) was added to the reaction. After stirring for 60 hours at room temperature, the reaction mixture was concentrated under reduced pressure. The residue was washed with saturated NaHCO₃. The aqueous phase was extracted with DCM(1×). The combined organic layers were dried with Na₂SO₄, filtered and concentrated. The residue was purified by silica gel chromatography (50 g, 20 m) using 0-10% MeOH/DCM. Pure fractions were combined, concentrated, and dried under high vacuum to give 0.7 g carbamate 4 (42%) as a white solid.

To a stirred solution of carbamate 4 (0.7 g, 0.64 mmol, 1 eq) and diisopropylethylamine (0.724 mL, 4.158 mmol, 6.5 eq) in anhydrous DCM (10 mL) was added N,N-diisopropyl chlorophosphoramidite (0.428 mL, 0.432 mmol, 3 eq) dropwise. The reaction was stirred for 2 hours at RT, LCMS shows the reaction is complete. The reaction was quenched with saturated NaHCO₃ solution and partitioned between DCM and saturated NaHCO₃. The DCM phase was collected. The aqueous phase was extracted with DCM (1×). The combined organic phases were dried over Na₂SO₄, filtered, and concentrated. The residue was loaded on to a pre-equilibrated (2% Et₃N-DCM) biotage silica gel column (25 g, m) and purified by flash chromatography using 0-10% MeOH/DCM containing 2% Et₃N as an additive. Pure fractions were combined, concentrated, and dried under high vacuum to give 610 mg (74%) phosphoramidite NB-101. MS: m/z=1316.1 [M+Na]⁺. P31-NMR and H1-NMR are corresponding with product.

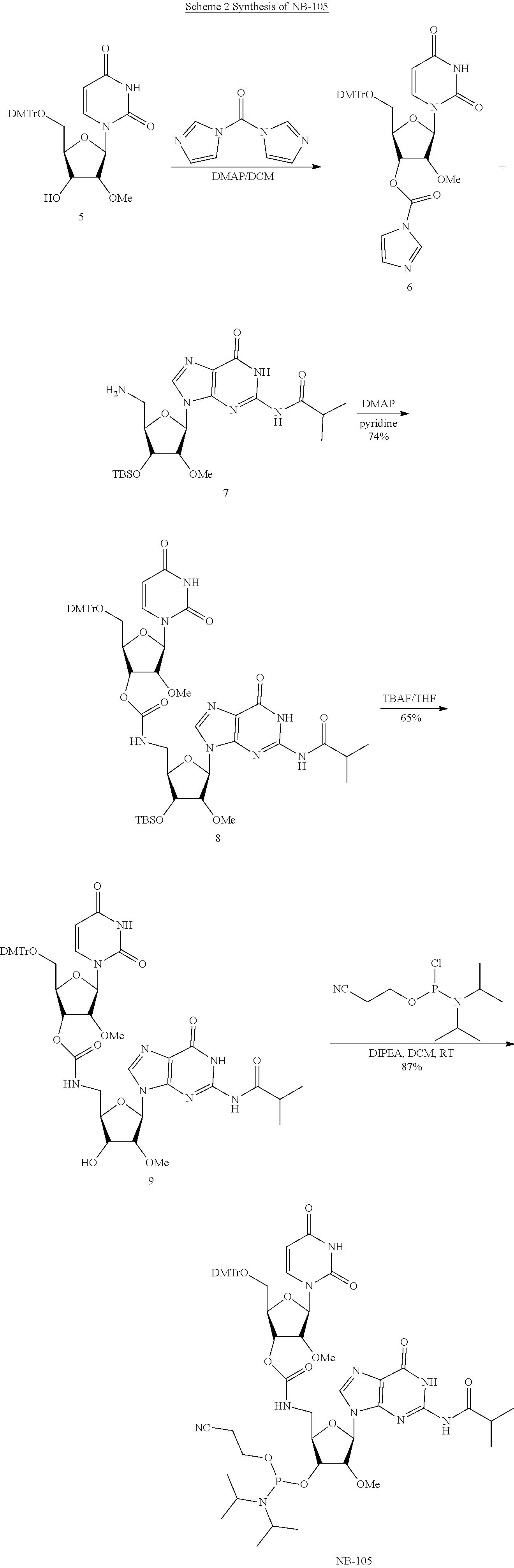

Alcohol 5 (1.0 g, 1.784 mmol, 1 eq), 1,1′ carbonyldiimidazole (0.578 mg, 3.568 mmol, 2 eq) and DMAP (0.436 mg, 3.568 mmol, 2 eq) were dissolved in dichloromethane (20 mL). After stirring overnight at room temperature, the reaction mixture was washed with saturated NH4Cl, and extracted with DCM(2×). The combined organic layers were dried with Na₂SO₄ and concentrated to give 1.2 g crude product 6 as a white solid which is used directly in the next step without purification.

Compound 6 (0.75 g, 1.146 mmol, 1 eq) and amine 7 (0.578 g, 1.203 mmol, 1.05 eq) were dissolved in anhydrous pyridine (20 mL) and evaporated to dryness. The residue was redissolved in anhydrous pyridine (15 mL). DMAP (140 mg, 1.146 mmol, 1 eq) was added to the reaction. After stirring for 18 hours at room temperature, the reaction mixture was concentrated under reduced pressure. The residue was partitioned between saturated NaHCO₃ and DCM. The aqueous phase was extracted with DCM(1×). The combined organic layers were dried with Na₂SO₄, filtered and concentrated. The residue was purified by silica gel chromatography (50 g, 20 m) using 0-10% MeOH/DCM. Pure fractions were combined, concentrated, and dried under high vacuum to give 0.91 g carbamate 8 (74%) as a white solid.

Carbamate 8 (530 mg, 0.497 mmol, 1 eq) was dissolved in THF (5 mL). TBAF (0.754 mL, 0.754 mmol, 1.5 eq) was added to the reaction. The reaction was stirred at RT for 2 hrs. LCMS shows still some SM left. Additional TBAF (0.251 mL, 0.251 mmol, 0.5 eq) was added to the reaction. The reaction was stirred for 1 hr. LCMS shows the reaction is complete. The reaction was concentrated under reduced pressure. The residue was purified by silica gel chromatography (25 g, 20 m) using 0-5-10% MeOH in DCM to give 309 mg product compound 9 (60%) as a white solid.

To a stirred solution of carbamate 9 (0.493 g, 0.517 mmol, 1 eq) and diisopropylethylamine (0.586 mL, 3.363 mmol, 6.5 eq) in anhydrous DCM (10 mL) was added 2-cyanoethyl N,N-diisopropyl chlorophosphoramidite (0.346 mL, 1.551 mmol, 3 eq) dropwise. The reaction was stirred for 1 hour at RT, LCMS shows still some carbamate 9 left. Additional 2-cyanoethyl N,N-diisopropyl chlorophosphoramidite (0.115 mL, 0.517 mmol, 1 eq) was added dropwise. The reaction was stirred at RT for 1 h. LCMS shows the reaction is complete. The reaction was quenched with saturated NaHCO₃ solution and partitioned between DCM and saturated NaHCO₃. The DCM phase was collected. The aqueous phase was extracted with DCM (2×). The combined organic phases were dried over Na₂SO₄, filtered, and concentrated. The residue was loaded on to a pre-equilibrated (1% Et₃N-DCM) biotage silica gel column (25 g, 20 m) and purified by flash chromatography using 0-10% MeOH/DCM containing 1% Et₃N as an additive. Pure fractions were combined, concentrated, and dried under high vacuum to give 520 mg (84%) phosphoramidite NB-105 (95% purity HPLC). MS: m/z=1152.9 [M]⁺. P31-NMR and H1-NMR are corresponding with product.

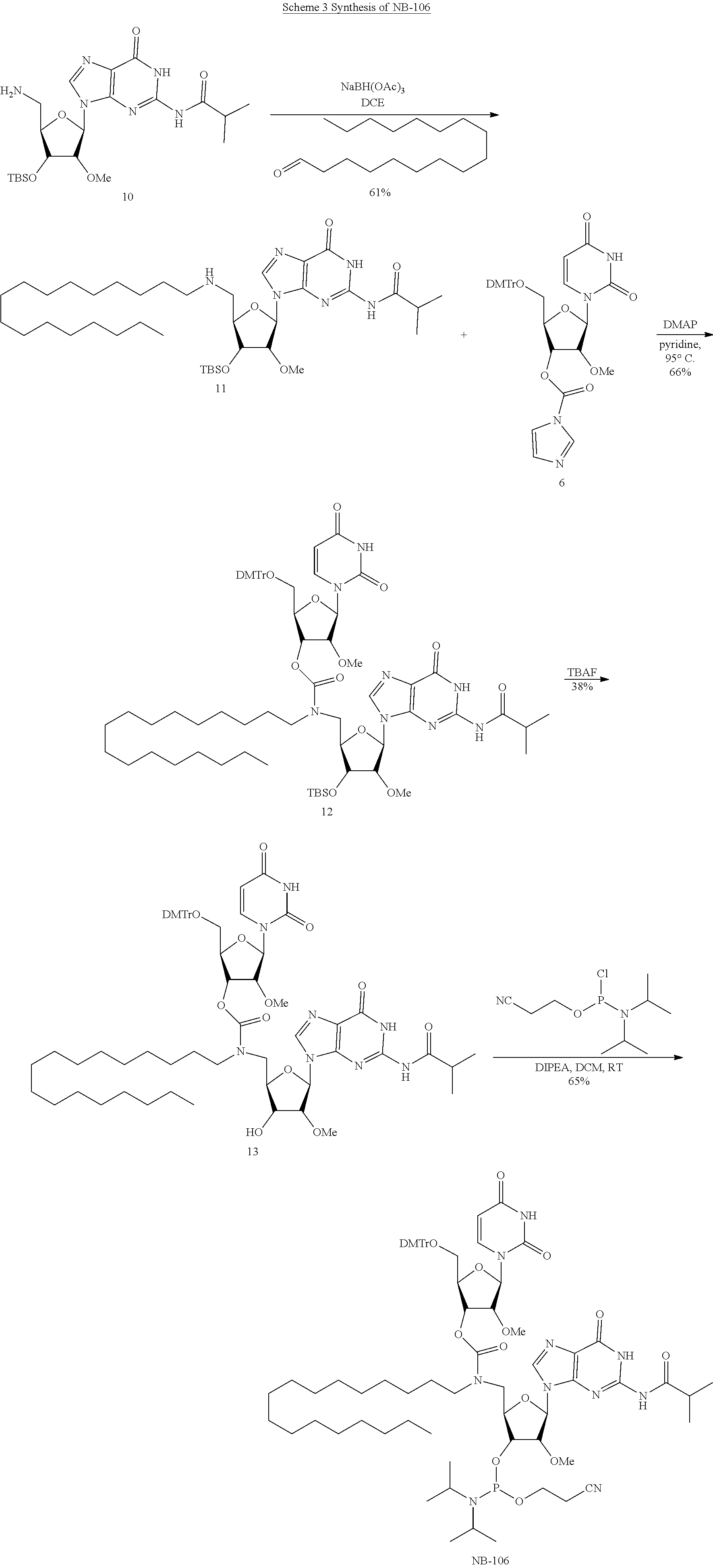

To a suspension of amine 10 (1.044 g, 2.172 mmol, 1 eq) and heptadecanal (0.608 g, 2.389 mmol, 1.1 eq) in DCE (50 mL) was added NaBH(OAc)₃ (0.691 g, 3.258 mmol, 1.5 eq) followed by 5 drops of AcOH. The reaction was stirred for 16 h at room temperature. LCMS shows small amount of SM left. 5 mL of MeOH was added to the reaction (to help with the solubility). The reaction was stirred for 3 hrs at RT. LCMS shows no change. The reaction was diluted with DCM and washed sequentially with NH₄Cl (1×) and NaHCO₃ (1×). The organic layer was dried with Na₂SO₄, filtered, and concentrated in vacuo to remove solvent. The residue was purified by silica gel chromatography (50 g, 60 m) using 0-15% MeOH/DCM (15 CV). Pure fractions were combined, concentrated, and dried under high vacuum to obtain 0.912 g 11 as a white solid.

Compound 6 (455 mg, 0.695 mmol, 1 eq) and amine 11 (400 mg, 0.556 mmol, 0.8 eq) were dissolved in anhydrous pyridine (3 mL). DMAP (85 mg, 0.695 mmol, 1 eq) was added to the reaction. The reaction was stirred at 95° C. for 40 hours. LCMS shows around 60% conversion of SM 6. The reaction mixture was concentrated under reduced pressure. The residue was partitioned between DCM and saturated NaHCO₃. The aqueous phase was extracted with DCM(1×). The combined organic layers were dried with Na₂SO₄, filtered and concentrated. The residue was purified by silica gel chromatography (25 g, 20 m) using 0-10% MeOH (w/1% Et₃N) in DCM to give 480 mg carbamate 12 (66%) as a slightly yellow solid. MS: m/z=1327.4 [M+Na]+.

Carbamate 12 (480 mg, 0.368 mmol, 1 eq) was dissolved in THF (5 mL). TBAF (0.735 mL, 0.735 mmol, 2 eq) was added to the reaction. The reaction was stirred at RT for 2 hrs. LCMS shows still some SM left. Additional TBAF (0.187 mL, 0.187 mmol, 0.5) was added to the reaction. The reaction was stirred for 1 hr. LCMS shows the reaction is complete. The reaction was concentrated under reduced pressure. The crude product was purified by silica gel chromatography (25 g, 20 m) (2×) using 0-10% MeOH in DCM to give 168 mg product 13 as a white solid. MS: m/z=1212.7 [M+Na]⁺

To a stirred solution of carbamate 13 (157 mg, 0.132 mmol, 1 eq) and diisopropylethylamine (0.149 mL, 0.857 mmol, 6.5 eq) in anhydrous DCM (5 mL) was added 2-cyanoethyl N,N-diisopropyl chlorophosphoramidite (0.088 mL, 0.395 mmol, 3 eq) dropwise. The reaction was stirred for 6 hours at RT, LCMS shows still about 6% carbamate 13 left. The reaction was stirred at RT for overnight. LCMS shows the reaction is complete (about 0.5% SM 13). The reaction was quenched with saturated NaHCO₃ solution and partitioned between DCM and saturated NaHCO₃. The DCM phase was collected. The aqueous phase was extracted with DCM (1×). The combined organic phases were dried over Na₂SO₄, filtered, and concentrated. The residue was loaded on to a pre-equilibrated (1% Et₃N-DCM) biotage silica gel column (25 g, 20 m) and purified by flash chromatography using 0-10% MeOH/DCM containing 1% Et₃N as an additive. Pure fractions were combined, concentrated, and dried under high vacuum to give 120 mg (65%) phosphoramidite NB-106. (92% purity HPLC. MS: m/z=1413.2 [M+Na]⁺. P31-NMR and H1-NMR are corresponding with product.

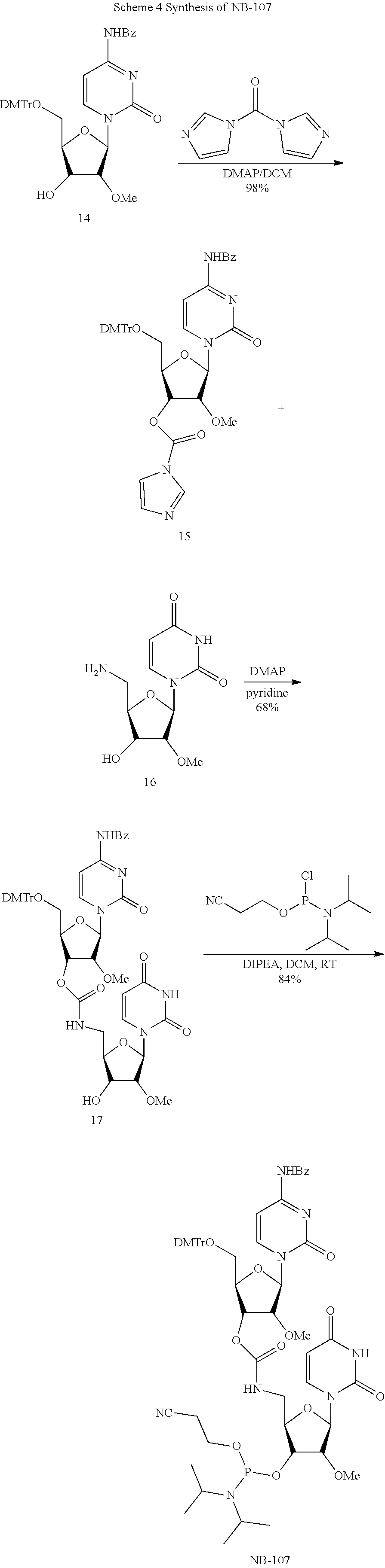

Alcohol 14 (1.0 g, 1.507 mmol, 1 eq), 1,1′ carbonyldiimidazole (0.489 g, 3.013 mmol, 2 eq) and DMAP (0.368 g, 3.013 mmol, 2 eq) were dissolved in dichloromethane (15 mL). After stirring for 14 h at room temperature, the reaction mixture was washed with saturated NH4Cl, and extracted with DCM(2×). The combined organic layers were dried with Na₂SO₄ and concentrated to give 1.2 g crude product 15 as a white solid which was used in the next step without purification.

Compound 15 (1.15 g, 1.518 mmol, 1 eq) and amine 16 (0.41 g, 1.593 mmol, 1.05 eq) were dissolved in anhydrous pyridine (12 mL). DMAP (185 mg, 1.518 mmol, 1 eq) was added to the reaction. After stirring for 20 hours, additional amine 16 (41 mg, 0.159 mmol, 0.1 eq) was added to the reaction, and the reaction was stirred for 3 hrs. The reaction mixture was concentrated under reduced pressure. The residue was partitioned between saturated NaHCO₃ and DCM. The aqueous phase was extracted with DCM(1×). The combined organic layers were dried with Na₂SO₄, filtered and concentrated. The residue was purified by silica gel chromatography (50 g, 20 m) using 0-10% MeOH/DCM. Pure fractions were combined, concentrated, and dried under high vacuum to give 0.97 g carbamate 17 (68%) as a white solid.

To a stirred solution of carbamate 17 (0.9 g, 0.95 mmol, 1 eq) and diisopropylethylamine (0.993 mL, 3.7 mmol, 6 eq) in anhydrous DCM (10 mL) was added 2-cyanoethyl N,N-diisopropyl chlorophosphoramidite (0.636 mL, 2.851 mmol, 3 eq) dropwise. The reaction was stirred for 45 min at RT, LCMS shows the reaction is complete. The reaction was quenched with saturated NaHCO₃ solution and partitioned between DCM and saturated NaHCO₃. The DCM phase was collected. The aqueous phase was extracted with DCM (2×). The combined organic phases were dried over Na₂SO₄, filtered, and concentrated. The residue was loaded on to a pre-equilibrated (1% Et₃N-DCM) biotage silica gel column (50 g, 20 m) and purified by flash chromatography using 0-10% MeOH/DCM containing 1% Et₃N as an additive. Pure fractions were combined, concentrated, and dried under high vacuum to give 920 mg (84%) phosphoramidite NB-107 (98% purity HPLC). MS: m/z=1152.9 [M]⁺. P31-NMR and H1-NMR are corresponding with product.

### Example 4: Synthesis of Triazole di-nucleotide 9

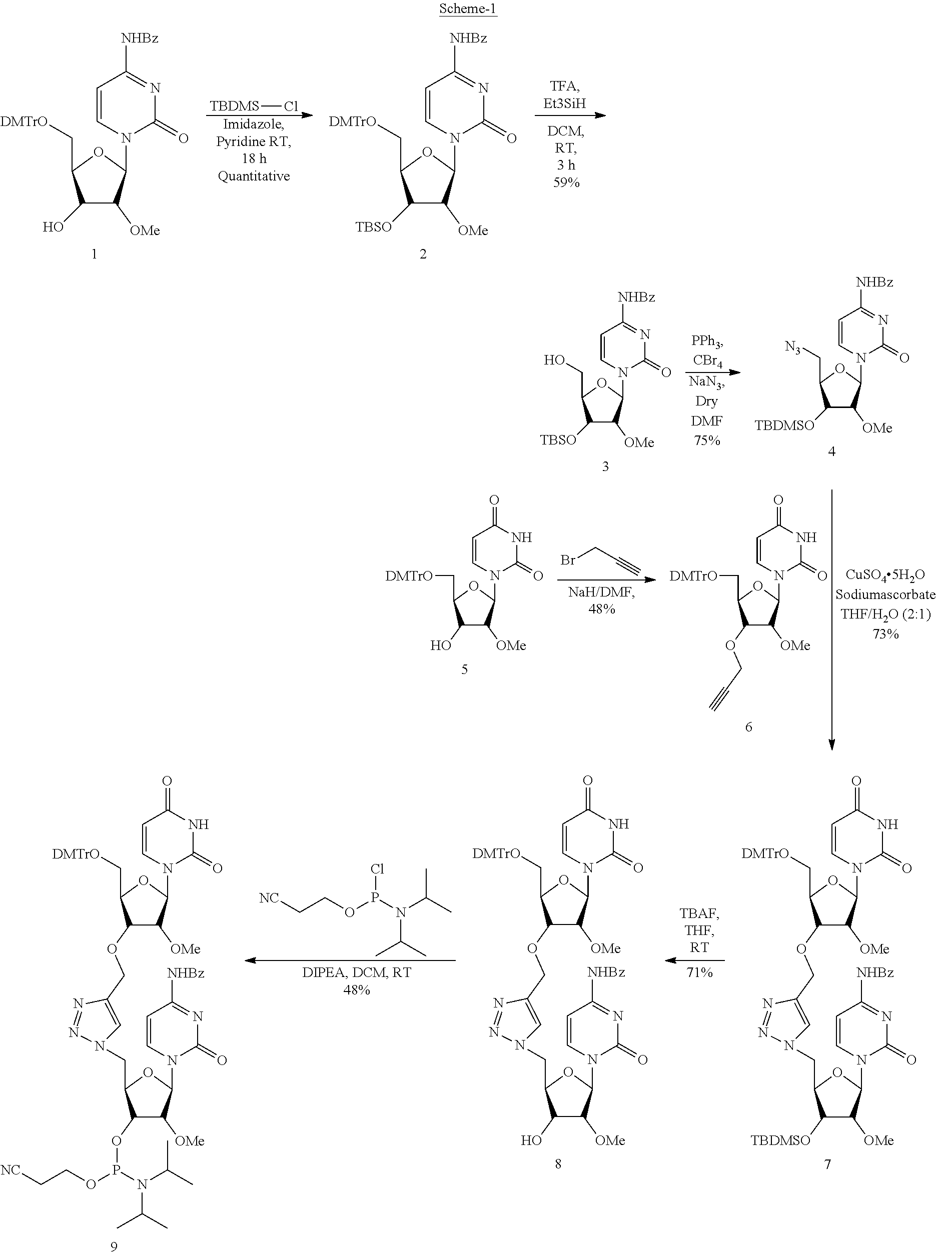

### Synthesis of 2

To a stirred solution of alcohol 1 (7.2 g, 10.86 mmol) in pyridine (25 ml.) was added Imidazole (1.9 g, 28.23 mmol, 2.6 eq) followed by TBDMS-Cl (2.12 g, 14.11 mmol 1.3 eq) the reaction mixture was stirred at RT for 12 h. Reaction mixture was diluted with DCM 100 ml washed with water 50 mL, brine solution 50 mL. Organic layer was concentrated, and the residue was co-evaporated with Toluene, dried under high vacuum to obtain TBS ether 2 as an orange solid (8.44 g, Quantitative) used as it is for next step. Product confirmed by LCMS. m/z 778 (M+1).

### Synthesis of 3

To a solution of compound 2 (8.44 g, 10.84 mmol, 1.00 eq.) in DCM (50 mL) was added TFA (1.82 mL, 23.86 mmol, 2.20 eq.). The color of the solution turned to red. Et₃SiH (1.9 mL, 11.93 mmol, 1.1 eq.) was added at 25° C. The reaction mixture was stirred at 25° C. for 5 h and the red solution became colorless. The solvent was removed under reduced pressure, and the residue was dissolved in EtOAc (100 mL). The organic phase was washed with NaHCO₃ (40 mL), brine, dried over Na₂SO₄, filtered and concentrated. The residue was purified by column chromatography on silica gel using 0-100 EtOAc/Hexane as an eluent, pure fractions were combined and concentrated to obtain alcohol 3 as a white solid (3.04 g 59%). Product confirmed by NMR and LCMS. M/z 476 (M+1).

### Synthesis of 4

Cytosine 5′-alcohol 3 (0.7 g, 1.474 mmol, 1 eq), triphenylphosphine (0.773 g, 2.947 mmol, 2 eq), and Sodium azide (0.383 g, 5.895 mmol, 4 eq) were suspended in dry DMF (20 mL) and carbon tetrabromide (0.977 g, 2.947 mmol, 2 eq) was added to the suspension. After stirring for 3 h at room temperature, the solvent was evaporated to concentrate. The residue was purified by column chromatography on silica gel with Ethyl acetate/Hexane 0-100% to give the azide 4 as a white foam (556 mg, 75%). NMR and LCMS m/z=501 (M+1) corresponding with the product.

### Synthesis of 6

To a stirred and cooled (0° C.) solution of alcohol 5 (3 g, 5.357 mmol, 1 eq) in anhydrous DMF (30 ml) was added NaH 60 w % (640 mg 16.071 mmol, 3 eq). reaction mixture was stirred at 0° C., for 30 min. Propargylbromide (0.876 mL, 5.893 mmol, 1.1 eq) was added dropwise, stirring was continued for 12 h at RT. Reaction mixture was quenched with water (50 mL), extracted with Ethyl acetate (200 mL), dried (Na₂SO₄) and concentrated, and the residue was purified by silica gel column chromatography using 0-50% Ethyl acetate/hexane as an eluent. Pure fractions were combined and concentrated to obtained alkyne 6 as a white solid (1.56 g, 48%). Product confirmed by NMR and LCMS m/z=597 (M−1).

### Synthesis of 7

A mixture of propargyl derivative 6 (658 mg, 1.1 mmol), azide 4 (550 mg, 1.1 mmol), CuSO₄-5H2O (1.099 g, 4.4 mmol), and sodium ascorbate (871 mg, 4.4 mmol) in THF/water 2:1 (v:v) (30 mL) was stirred at room temperature under an argon atmosphere for 12 h. The mixture was diluted with EtOAc (100 mL) The organic layer was washed with aqueous Saturated NaHCO₃ solution (50 ML), brine solution (50 mL), dried over Na₂SO₄ and concentrated. The crude product was purified by column chromatography, using EtOAc/Hexane 0-100% as an eluent, to obtain triazole 7 as a white solid (875 mg, 73%). Product confirmed by NMR and LCMS, m/z=1100 (M+1).

### Synthesis of 8

To attired solution of TBDMS ether 7 (0.87 g, 0.792 mmol, 1 eq) in anhydrous THF (10 mL) was added TBAF (0.275 g, 0.871 mmol, 1.1 eq). After stirring at room temperature for 48 hours, the reaction was poured into EtOAc and the organic phase was sequentially washed with H₂O, saturated NaHCO₃, brine, dried (Na₂SO₄) and concentrated under vacuum. Purification by column chromatography (SiO₂, eluting with 0-5% MeOH/EtOAc to obtain 3′-Alcohol 8 as a white solid (552 mg 71%). Product confirmed by NMR and LCMS m/z=985 (M+).

### Synthesis of 9

Alcohol 8 (519 mg, 0.477 mmol, 1 eq) was dissolved in 10 mL of methylene chloride, then diisopropylethylamine (0.2 mL, 1.144 mmol, 2.4 eq), followed by Cyanoethyl N,N-diisopropyl chlorophosphoramidite (0.12 mL 0.572 mmol, 1.2 eq) was added dropwise. The reaction mixture was stirred at room temperature for 2 hours. LCMS showed no product formation, then another portion of diisopropylethylamine (0.2 mL, 1.144 mmol, 2.4 eq), followed by Cyanoethyl N,N-diisopropyl chlorophosphoramidite (0.12 mL 0.572 mmol, 1.2 eq) was added dropwise and the mixture was stirred for 2 h at room temperature. LCMS showed complete conversion and product showed 1101 mass (de-fragmented diisopropylamine). Reaction mixture was diluted with hexane to get the precipitation, the solvent was decanted, and the residue retained in the flask was dried under high vacuum. The crude product was purified by Biotage silica gel column 10 gr 20 micron, in two batches (250 mg each) using 1% Et₃N-DCM/Ethyl acetate 0-100% (0-100% 4CV, 100% EA 8CV). Pure fractions were combined and concentrated from two batches, dried under high vacuum to obtain Phosphoramidate 9 (302 mg, 48%) as a white solid. 85% purity based on 31P NMR. 31P, H1-NMR and LCMS m/z 1101 corresponding with the product structure.

### Example 5: Synthesis of Acid Monomers Experimental

### Synthesis of mU-3′-Acid

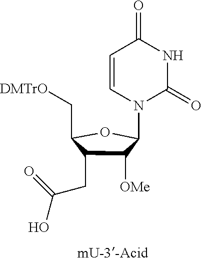

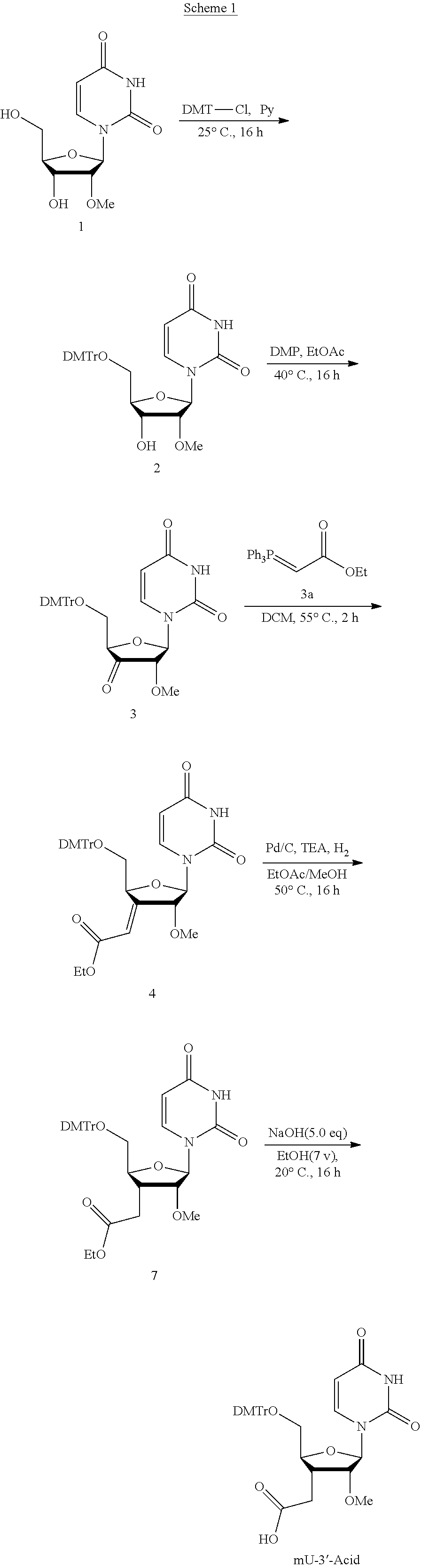

### Experimental

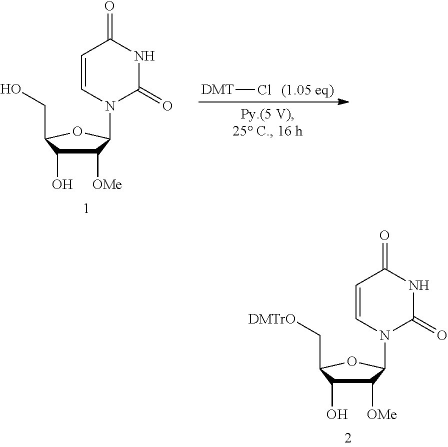

To a solution of compound 1 (300 g, 1.16 mol) and DMTr-Cl (413 g, 1.22 mol) was added Py (1.50 L) with stirring at 25° C. The mixture was refluxed at 25° C. for 16 hrs. LCMS (ET54837-4-P1L2, compound 2: Rₜ=2.52 min) showed the reaction was complete. Using the similar conditions another two batches (2×300 g) were carried in parallel, after completing the reaction the three reactions mixtures were combined and quenched by H2O (4.00 L) and extracted with DCM (3.00 L×3). The combined organic layers were washed with brine (3.00 L), dried over Na₂SO₄, filtered, and concentrated under reduced pressure. The residue was purified by column chromatography (SiO₂, Petroleum ether/Ethyl acetate=50/1 to 2/1) to obtain compound 2 (1.60 kg, 73.7% yield, 90.0% purity) as a white solid.

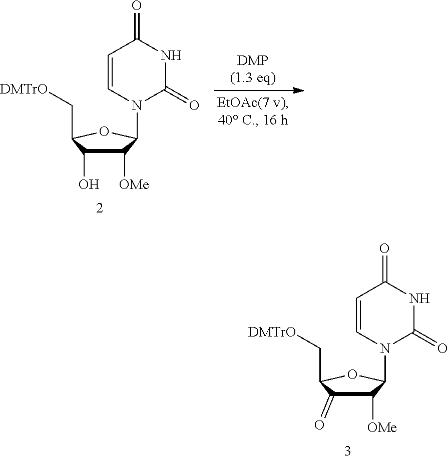

A mixture of compound 2 (200 g, 356 mmol) and DMP (196 g, 463 mmol) in EtOAc (1.40 L) was degassed and purged with N₂ for 3 times, and then the mixture was stirred at 40° C. for 16 hrs under N₂ atmosphere. TLC (Petroleum ether/Ethyl acetate=0/1, Compound 2: R_{f}=0.4, Compound 3: R_{f}=0.5) showed the reaction was complete. Using the similar conditions another 200 g batch was carried out in parallel, after completing the reaction two batches were combined and filtered, the filtrate was quenched by sat. NaHCO₃ (3.00 L) and extracted with EtOAc (2.00 L). The combined organic layers were washed with brine (3.00 L), dried over Na₂SO₄, filtered, and concentrated under reduced pressure, to give compound 3 (400 g, crude) as yellow oil and used as it is for next step.

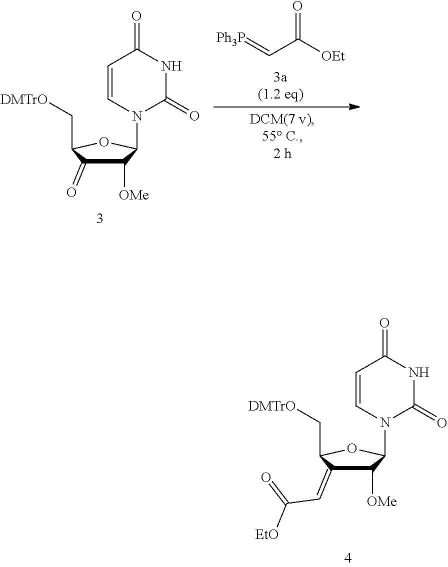

A mixture of compound 3 (200 g, 358 mmol) and compound 3a (149 g, 429 mmol) in DCM (1.40 L) was degassed and purged with N₂ for 3 times, and then the mixture was stirred at 55° C. for 2 hrs under N₂ atmosphere. LCMS (ET55113-28-P1A1, compound 4: Rₜ=0.848 min) showed the reaction was complete. Using the similar conditions another 200 g batch was carried out in parallel, after completing the reaction two batches were combined and filtered. The filtrate was concentrated under reduced pressure to give a residue. The residue was purified by prep-HPLC (neutral condition) to obtain compound 4 (270 g, 53.9% yield, 90.0% purity) as brown oil. ¹HNMR: (400 MHz, CDCl₃) δ 8.57 (s, 1H), 8.01 (d, J=8.4 Hz, 1H), 7.34-7.21 (m, 9H), 6.83-6.80 (m, 4H), 6.17-6.08 (m, 2H), 5.38-5.32 (m, 2H), 4.71-4.72 (m, 1H), 4.14-4.12 (m, 2H), 3.91 (d, J=1.2 Hz, 1H), 3.79-3.78 (m, 6H), 3.53 (s, 3H), 3.35 (d, J=2.0 Hz, 1H), 1.27-1.25 (m, 3H).

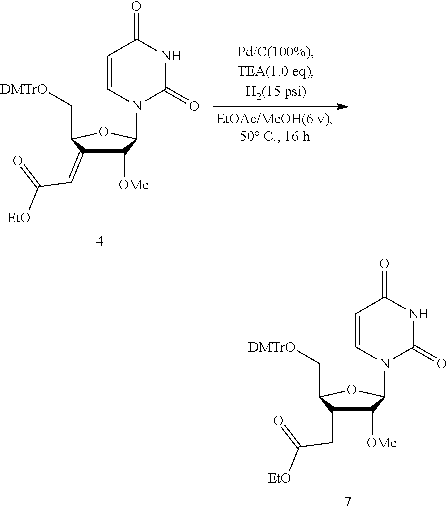

To a solution of compound 4 (30.0 g, 47.7 mmol) and TEA (4.83 g, 47.7 mmol, 6.64 mL) in EtOAc (60.0 mL) and MeOH (60.0 mL) was added Pd—C(10%, 30.0 g) under Ar. The suspension was degassed under vacuum and purged with H₂ several times. The mixture was stirred under H₂ (15 psi) at 50° C. for 16 hrs. LCMS (ET55113-32-P1A, compound 7: Rₜ=0.840 min) showed the starting material was consumed completely. Using the similar conditions another 3×30 g batches were carried out in parallel, after completing the reaction four batches were combined and filtered and the filter was concentrated. The crude product was triturated with MTBE (100 mL) at 15° C. for 30 mins. To obtain compound 7 (110 g, 91.3% yield) was obtained as a white solid. ¹H NMR: ET55113-32-P1A (400 MHz, CDCl₃), δ 8.22 (d, J=8.0 Hz, 1H), 7.40-7.27 (m, 9H), 6.86-6.83 (m, 4H), 5.94 (s, 1H), 5.33-5.30 (m, 1H), 4.12-4.10 (m, 2H), 3.94-3.93 (m, 2H), 3.84 (m, 7H), 3.80-3.79 (m, 1H), 3.53 (s, 3H), 3.28-3.25 (m, 1H), 2.54-2.51 (m, 1H), 2.50-2.47 (m, 1H), 2.00-1.96 (m, 1H), 1.26-1.24 (m, 3H).

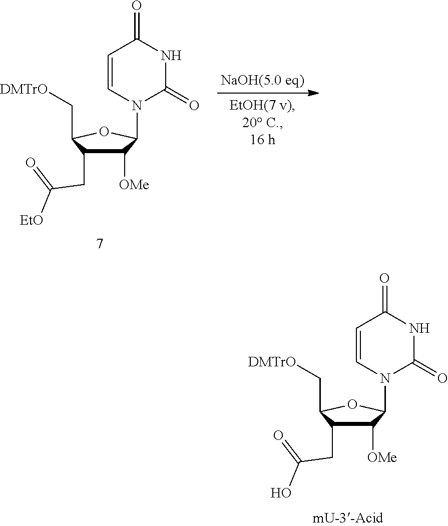

A mixture of compound 7 (120 g, 190 mmol, 1.00 eq) and NaOH (38.0 g, 951 mmol) in EtOH (840 mL) was degassed and purged with N₂ for 3 times, and then the mixture was stirred at 20° C. for 16 hrs under N₂ atmosphere. LCMS (ET55113-34-P1A, mU-3′-Acid: Rₜ=0.744 min) showed the reaction was complete. Reaction mixture was diluted with H₂O (1.00 L) and adjusted to pH=6 with aq·HCl (2.0 M, 400 mL). Precipitated white solid was filtered, the filter cake was dried under reduced pressure. The crude product was triturated with MTBE (300 mL) at 15° C. to obtain mU-3′-Acid (101.6 g, 87.1% yield, 98.3% purity) was obtained as white solid. ¹HNMR of mU-3′-Acid (400 MHz, DMSO-d₆), δ 11.39 (br, 1H), 7.93 (d, J=8.0 Hz, 1H), 7.38-7.23 (m, 9H), 6.91-6.89 (m, 4H), 5.75 (s, 1H), 5.18 (d, J=8.0 Hz, 1H), 3.89-3.88 (m, 2H), 3.74 (s, 6H), 3.44-3.38 (m, 4H), 3.28-3.23 (m, 1H), 2.34-2.27 (m, 1H), 2.43-2.31 (m, 1H), 2.00-1.98 (m, 1H).

### Synthesis of ADAR-015

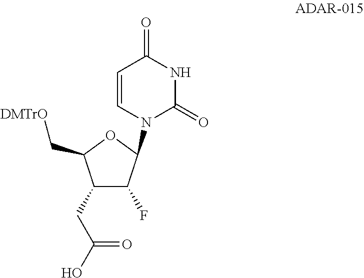

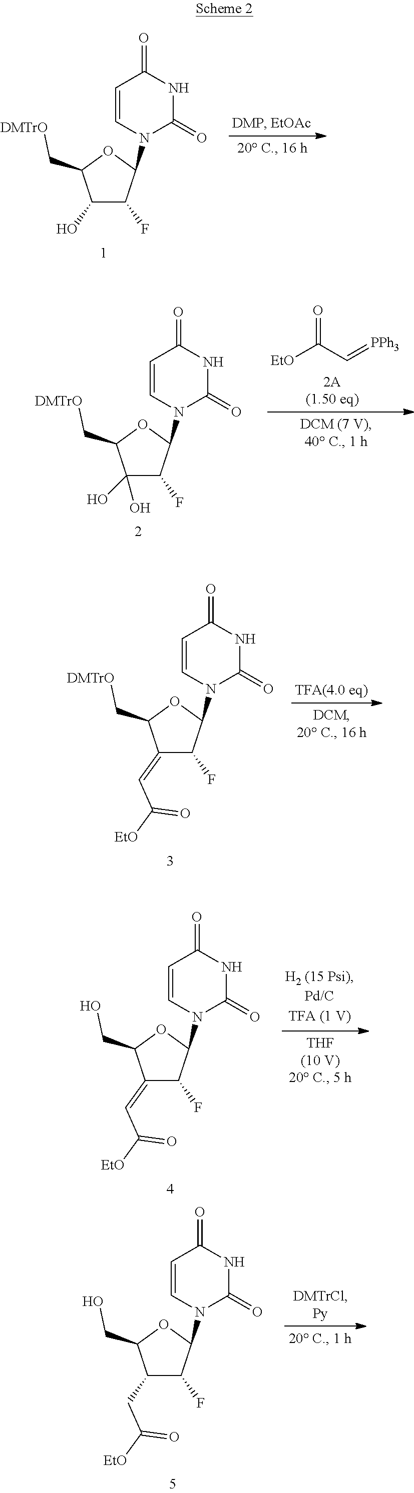

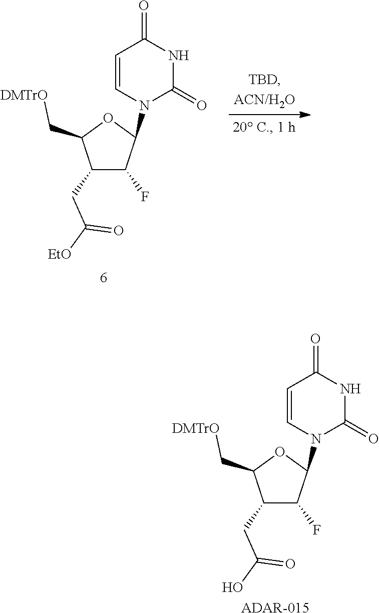

### Experimental

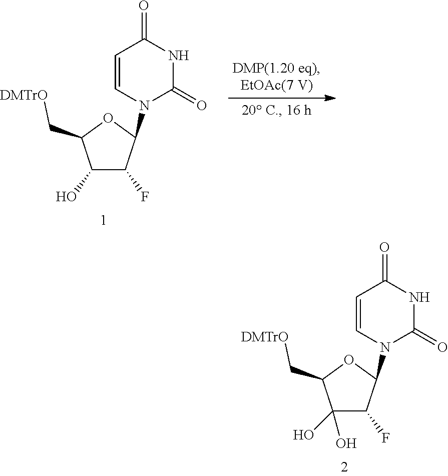

To a solution of compound 1 (200 g, 364 mmol, 1.00 eq) in EtOAc (1.40 L) was added DMP (185 g, 437 mmol, 135 mL, 1.20 eq). The mixture was stirred at 20° C. for 16 h. LCMS (ET60022-65-P1L1, Cpd. 2: Rₜ=0.897 min) showed Cpd. 2 formed. Using the similar conditions another 200 g batch was carried out in parallel, after completing the reaction two batches were combined and treated with sat. NaHCO₃ (4000 mL). Stirred at 20° C. for 2 hrs. Then the mixture was filtered, filtrate was extracted with EtOAc (1000 mL×3) and dried over anhydrous Na₂SO₄, concentrate in vacuum to obtain compound 2 (400 g, crude) as yellow oil.

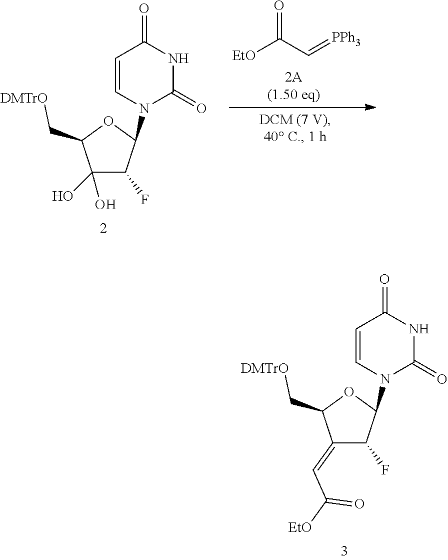

To a solution of compound 2 (200 g, 354 mmol, 1.00 eq) in DCM (1400 mL), was added compound 2A (185 g, 531 mmol, 1.50 eq) the mixture was stirred at 40° C. for 1 h. LCMS (ET60022-67-P1L2, Cpd. 3: Rₜ=2.286 min) showed Cpd. 3 formed. TLC (Petroleum ether/Ethyl acetate=1/1, R_{f}=0.80) showed Cpd. 3 formed. Using the similar conditions another 200 g batch was carried out in parallel, after completing the reaction two batches were combined, filtered, and the filtrate was concentrated in vacuum. Purified by prep-MPLC (acetonitrile+H₂O) to obtain compound 3 (260 g, 398 mmol, 56.3% yield, 94.6% purity) as yellow oil.

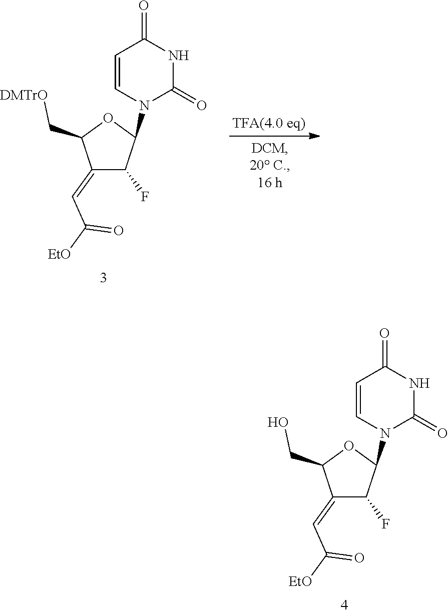

To a solution of compound 3 (130 g, 210 mmol, 1.00 eq) in DCM (910 mL), was added TFA (96.1 g, 843 mmol, 62.4 mL, 4.00 eq). The mixture was stirred at 20° C. for 16 h. TLC (Petroleum ether:Ethyl acetate=1:1) shows Cpd. 4 formed. Sat·NaHCO₃ (1000 mL) was added to the mixture. The organic layer was separated, aqueous layer was extracted with THF (500 mL×5). Combined organic layers were concentrated in vacuum, resulting crude product was purified by chromatography on a silica gel eluted with petroleum ether:ethyl acetate (from 100/1 to 0/1) to obtain compound 4 (60.0 g, 190 mmol, 90.5% yield) as a white solid.

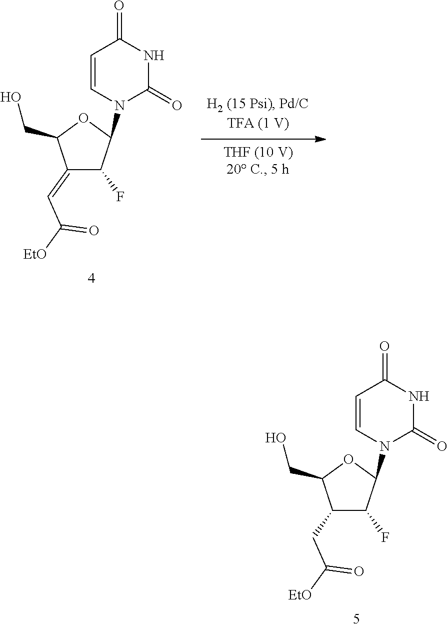 [SMILES: CCOC(=O)/C=C1\[C@@H](F)[C@H](n2ccc(=O)[nH]c2=O)O[C@@H]1CO ; CCOC(=O)C[C@H]1[C@@H](F)[C@H](n2ccc(=O)[nH]c2=O)O[C@@H]1CO]

To a solution of Pd/C (60.0 g, 10% purity) in THF (1.20 L) was added compound 4 (120 g, 381 mmol, 73.6% purity) and TFA (184 g, 1.62 mol, 120 mL) at 20° C. The suspension was degassed under vacuum and purged with H₂ three times, the mixture was stirred at 20° C. for 5 hours under H₂ (15 psi). LCMS (ET60022-82-P1LC, compound 6: Rₜ=1.117 min) showed compound 5 was consumed and compound 6 was formed. The mixture was filtered, and then sat. NaHCO₃ (2.00 L) and NaCl solid (10 g) were added to the filtrate and the resulting mixture was extracted with THF (500 mL×3). The organic layers were combined, dried with anhydrous Na₂SO₄ and concentrated in vacuum to have white solid precipitated, then the mixture was filtered and the filter cake was collected and dried to give compound 5 (45.0 g, 37.2% yield) as white solid.

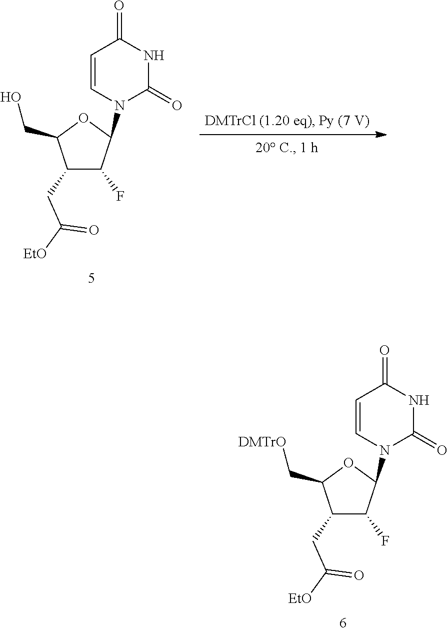

Compound 5 (45.0 g, 142 mmol, 1.00 eq) was added to Py (315 mL). Then DMTrCl (57.8 g, 170 mmol, 1.20 eq) was added. The mixture was stirred at 20° C. for 1 h. LCMS (ET60022-84-P1L1, Cpd. 4: RT=2.263 min) showed Cpd. 4 was formed. The mixture was concentrated in vacuum. Then water (1000 mL) and EtOAc (200 ml) was added to the mixture. Then extracted with EtOAc (200 mL×3). The organic layer was dried with anhydrous Na₂SO₄. The mixture was concentrated in vacuum. The combined crude product was purified by chromatography on a silica gel eluted with petroleum ether:ethyl acetate (from 100/1 to 0/1) to obtain compound 6 (70.4 g, 80%, 98.2% purity) as yellow oil.

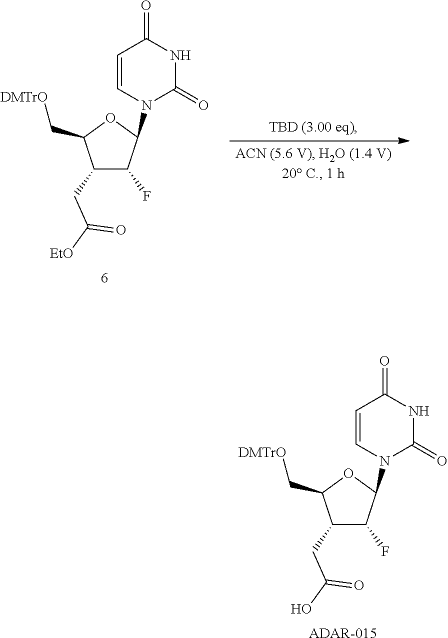

To a mixture of compound 6 (43.7 g, 70.6 mmol, 1.00 eq) in ACN (240 mL) and H₂O (60 mL) was added TBD (29.5 g, 211 mmol, 3.00 eq). The mixture was stirred at 20° C. for 1 h. LCMS (ET60022-76-P1L1, ADARX-015: RT=1.605 min) showed ADAR-015 formed. Water (300 mL) and DCM (300 mL) was added to the mixture. Then citric acid (10%) was added to the mixture to adjust the pH is 5-6. Then the mixture was extracted with DCM (100 mL×3). The organic layer was separated and concentrated in vacuum to obtain ADAR-015 (18.0 g, 44%, 98.5% purity) as a white solid. ¹HNMR: ET60022-76-P1NJ (400 MHz, DMSO-d₆), δ 11.4 (s, 1H), 9.71 (s, 1H), 7.86 (d, J=8.0 Hz, 1H), 7.40-7.33 (m, 1H), 7.39-7.30 (m, 2H), 7.27-7.25 (m, 4H), 6.91-6.88 (m, 4H), 5.85 (d, J=19.2 Hz, 1H), 5.23-5.21 (m, 1H), 3.98-3.74 (m, 1H), 3.41-3.25 (m, 6H), 3.24 (s, 3H), 3.11-3.10 (m, 1H), 2.26-2.23 (m, 1H), 2.07-2.03 (m, 1H), 1.87-1.83 (m, 2H).

### Synthesis of mC-3′-Acid

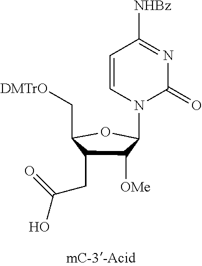

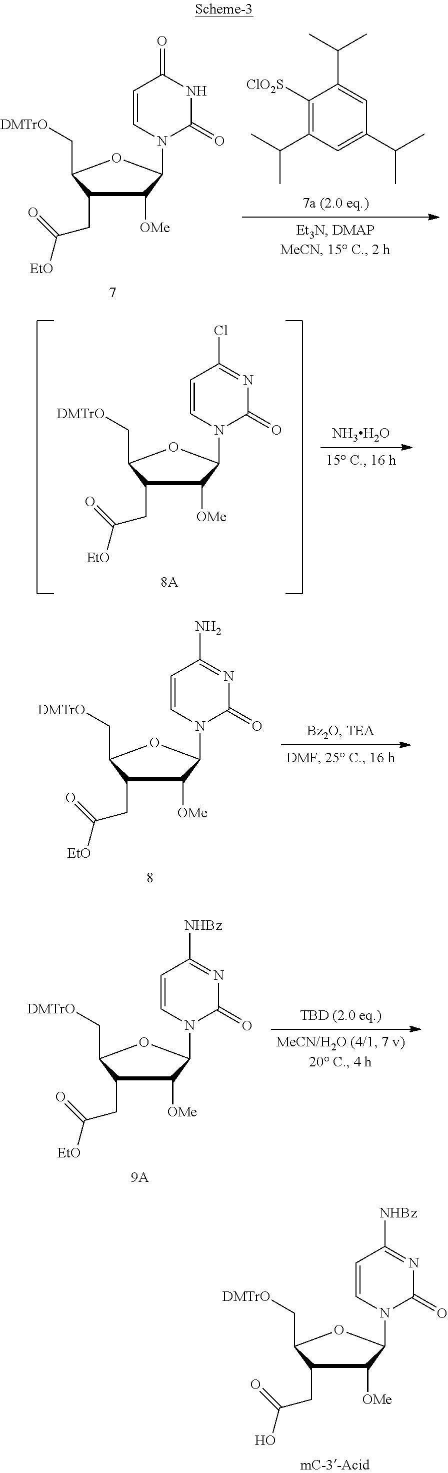

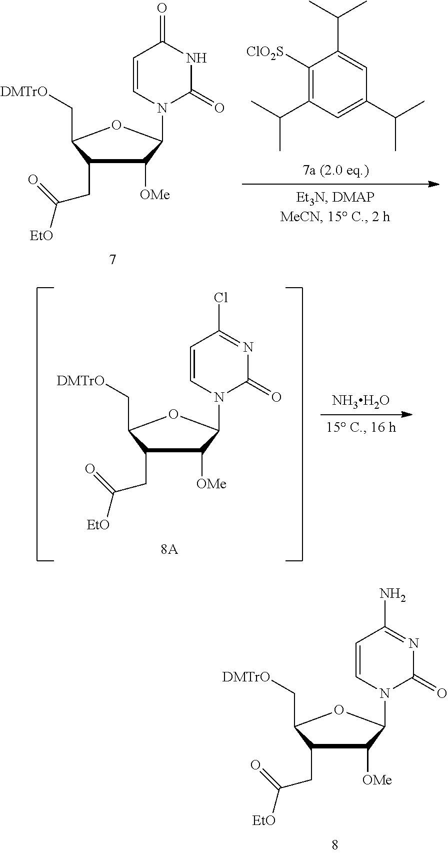

To a solution of compound 7 (100 g, 158 mmol) in MeCN (700 mL) was added TEA (32.0 g, 317 mmol, 44.1 mL), DMAP (38.7 g, 317 mmol) and compound 7a (96.0 g, 317 mmol), Then the mixture was stirred at 15° C. for 2 hrs under N₂ atmosphere. TLC (Petroleum ether/Ethyl acetate=0/1, Compound 7: R_{f}=0.4, Compound 8A: R_{f}=0.0) showed the formation of Chloro-derivative 8A. To the above solution of compound 8A (100 g, 158 mmol) was added NH₃·H₂O (455 g, 3.64 mol, 500 mL, 28.0% purity, 23.6 eq), Then the mixture was stirred at 15° C. for 16 hrs under N₂ atmosphere. LCMS (ET55113-59-P1A, compound 8: Rₜ=0.916 min) showed the reaction was complete. The reaction mixture was concentrated under reduce pressure to remove MeCN. The residue was quenched by addition sat. NH₄Cl (300 mL) at 15° C., and then diluted with DCM (200 mL) and extracted with DCM (300 mL×2). The combined organic layers were washed with brine 300 mL, dried over Na₂SO₄, filtered, and concentrated under reduced pressure to give a residue. The crude product was purified by prep-HPLC (neutral condition). To obtain compound 8 (75.0 g, 77.3% yield) as white solid. ¹HNMR: ET55113-59-P1B (400 MHz, DMSO-d₆), δ 8.05 (d, J=7.6 Hz, 1H), 7.39-7.23 (m, 11H), 6.91-6.89 (m, 4H), 5.78 (s, 1H), 5.53 (d, J=7.2 Hz, 1H), 4.03-4.00 (m, 2H), 3.99-3.96 (m, 1H), 3.91-3.77 (m, 7H), 3.48-3.46 (m, 1H), 3.42 (s, 3H), 3.21-3.20 (m, 1H), 2.56-2.54 (m, 1H), 2.38-2.36 (m, 1H), 1.96-1.93 (m, 1H), 1.14-1.10 (m, 3H).

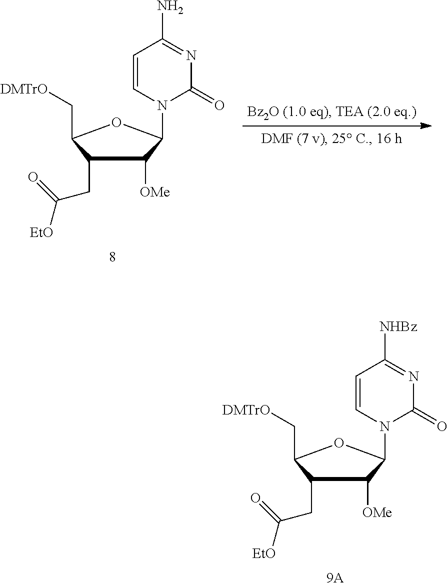

A mixture of compound 8 (105 g, 166 mmol), TEA (33.7 g, 333 mmol, 46.4 mL) and Bz₂O (37.7 g, 166 mmol, 31.4 mL) in DMF (735 mL) was degassed and purged with N₂ for 3 times, and then the mixture was stirred at 30° C. for 16 hrs under N₂ atmosphere. LCMS (ET55113-73-P1C, compound 9A: Rₜ=0.913 min) showed the reaction was complete. Reaction mixture was quenched by sat·NaHCO₃ (2.00 L) and extracted with EtOAc (800 mL×2). The combined organic layers were washed with brine (1.00 L), dried over Na₂SO₄, filtered and concentrated under reduced pressure. The residue was purified by column chromatography (SiO₂, Petroleum ether/Ethyl acetate=I/O to 0/1). To obtain compound 9A (110 g, 89.9% yield) as yellow oil. ¹HNMR: ET55113-73-P1A (400 MHz, DMSO-d₆), δ 8.59 (d, J=7.6 Hz, 1H), 8.01 (d, J=7.2 Hz, 1H), 7.95 (s, 1H), 7.71-7.29 (m, 13H), 6.94-6.92 (m, 4H), 5.87 (s, 1H), 4.03-4.01 (m, 3H), 3.99-3.91 (m, 1H), 3.75 (s, 6H), 3.55-3.49 (m, 4H), 3.34-3.32 (m, 1H), 3.64-2.61 (m, 1H), 2.45-2.40 (m, 1H), 1.98-1.96 (m, 1H), 1.16-1.13 (m, 3H).

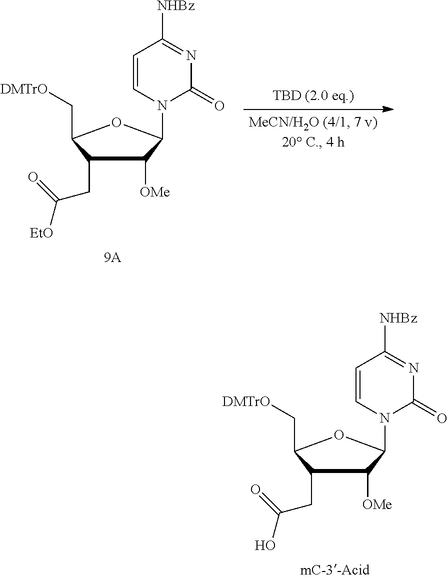

To a mixture of compound 9A (18.0 g, 24.5 mmol, 1 eq) in MeCN (100 mL) and H₂O (26.0 mL) was added (Triazabicyclodecene) TBD (6.83 g, 49.0 mmol) at 20° C. The mixture was stirred at 20° C. for 4 hrs. LCMS (ET55113-76-P1A1, mC-3′-Acid: Rₜ=0.612 min) showed the reaction was complete. The mixture was dilute with DCM (500 mL) and adjusted to pH=6 with aq·citric (10%, 200 mL). The mixture was separated, and the organic layer was washed with brine (200 mL), and dried over Na₂SO₄, filtered and concentrated under reduced pressure to give a residue. The residue was purified by triturated with MTBE (200 mL) at 15° C. for 30 mins to give mC-3′-Acid (10.8 g, 62% yield, 96.1% purity) as a white solid. LCMS: m/z 705 (M′), ¹H NMR: ET55113-76-P1L1 (400 MHz, DMSO-d₆), δ 12.32 (br, 1H), 11.32 (br, 1H), 8.58 (d, J=8.0 Hz, 1H), 7.98 (d, J=8.4 Hz, 2H), 7.61-7.26 (m, 13H), 6.92-6.90 (m, 4H), 5.84 (s, 1H), 4.00-4.96 (m, 1H), 3.89-3.88 (m, 1H), 3.74 (s, 6H), 3.51-3.48 (m, 4H), 3.28-3.25 (m, 1H), 2.50-2.48 (m, 1H), 2.32-2.30 (m, 1H), 1.89-1.85 (m, 1H).

### Synthesis of ADAR-018

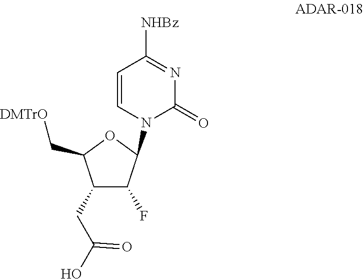

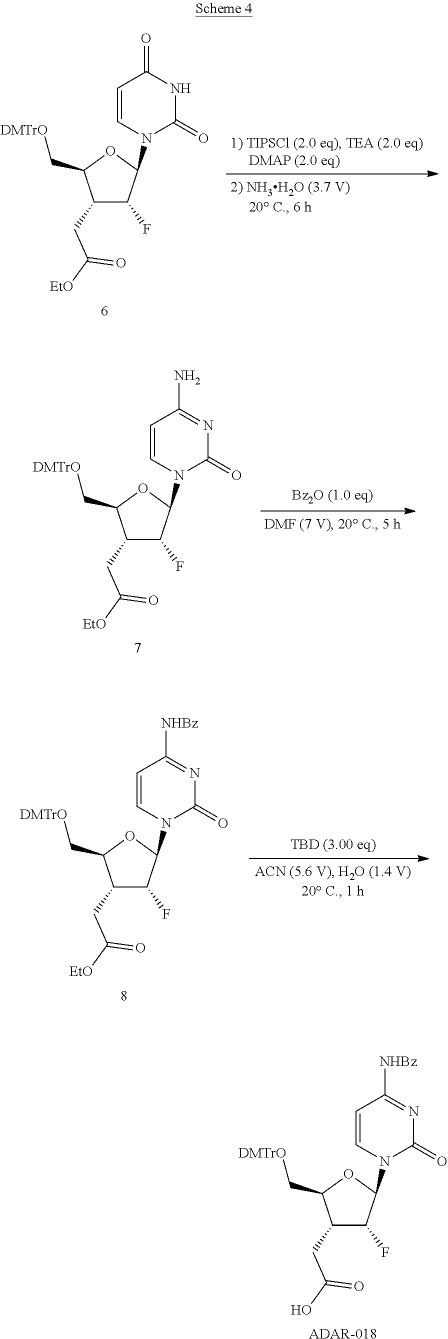

### Experimental

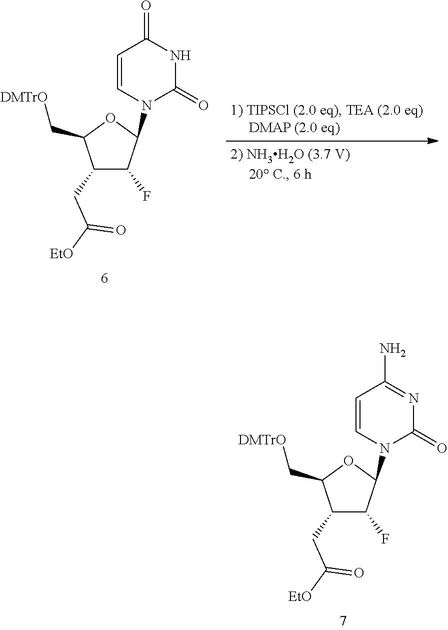

To a mixture of compound 6 (115 g, 185 mmol, 1.00 eq) in ACN (700 mL). was added TEA (37.6 g, 371 mmol, 51.7 mL, 2.00 eq). TIPSCl (112 g, 371 mmol, 2.00 eq) and DMAP (45.4 g, 371 mmol, 2.00 eq) then the mixture was stirred at 20° C. for 3 h. Then NH₃·H₂O (387 g, 3.20 mol, 425 mL, 29% purity, 17.2 eq) was added to the mixture and stirring was continued at 20° C. for 3 h. LCMS (ET60022-95-P1L2, Cpd. 7: Rₜ=2.173 min) shows Cpd. 7 formed. Reaction mixture was quenched with brine (500 mL), extracted with DCM (500 mL×3). The organic layer was separated and concentrated in vacuum, purified by prep-MPLC (ACN+H₂O). Then concentrated in vacuum to remove acetonitrile. Then aqueous phase was extracted with DCM (1000 mL×3), and the organic layer was concentrated in vacuum to obtain compound 7 (103 g, 158 mmol, 85.4% yield) as white solid.

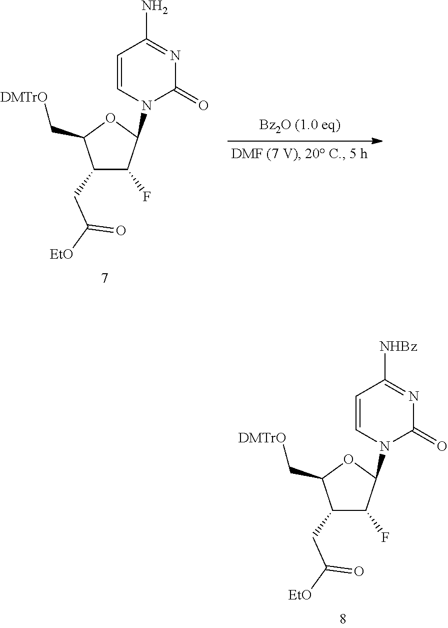

To a solution of compound 7 (103 g, 166 mmol, 1.00 eq) in DMF (721 mL) was added benzoic anhydride (37.7 g, 166 mmol, 31.4 mL, 1.00 eq), the mixture was stirred at 20° C. for 5 h. LCMS (ET60022-97-P1L1, Cpd. 8: Rₜ=2.488 min) showed Cpd. 8 formed. Brine (1500 mL) was added to the mixture and extracted with EtOAc (500 mL×2). The organic layer washed with sat. NaHCO₃ (400 ml×2). dried with anhydrous Na₂SO₄, and concentrated in vacuum to obtain compound 8 (120 g, 156 mmol, 93.7% yield).

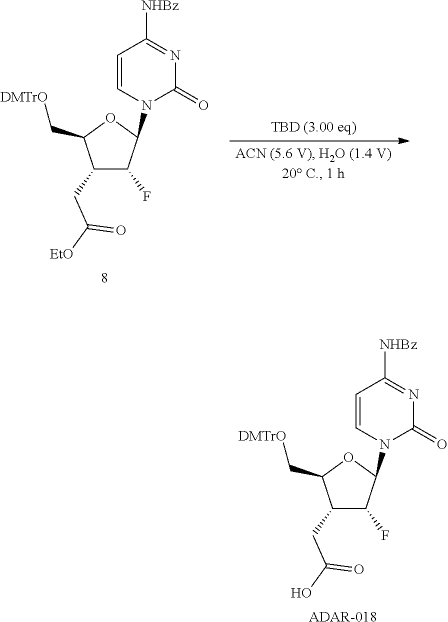

To a solution of compound 8 (120 g, 166 mmol, 1.00 eq) in ACN (672 mL) and H₂O (168 mL). was added TBD (69.4 g, 498 mmol, 3.00 eq). The mixture was stirred at 20° C. for 1 h. TLC (Petroleum ether:Ethyl acetate=1:1, ADAR-018: R_{f}=0.10) showed Cpd. 8 consumed. Citric acid (10%) was added to the mixture to adjust the pH 5-6. Precipated solids were filtered the filter cake was washed with deionized water (500 mL×3). The solid was concentrated in vacuum, and freeze-dried to obtain ADAR-018 (85.6 g, 122 mmol, 73.4% yield, 99.0% purity) as a white solid. LCMS: m/z, 693 (M′), ¹H NMR: (400 MHz, DMSO-d₆), δ 12.5 (s, 1H), 11.3 (s, 1H), 8.44 (s, 1H), 7.98 (d, J=8.4 Hz, 2H), 7.54-6.93 (m, 13H), 6.92-6.90 (m, 4H), 5.99 (s, 1H), 5.33-5.20 (m, 1H), 4.12 (m, 1H), 3.55 (s, 6H), 3.25 (s, 1H), 2.76 (s, 1H), 2.35-2.32 (m, 1H), 2.32-2.01 (m, 1H).

### Synthesis of ADARx-013

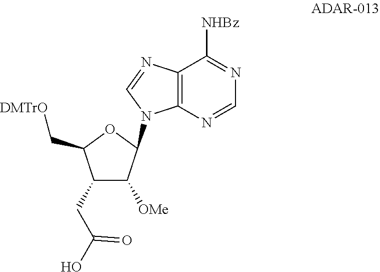

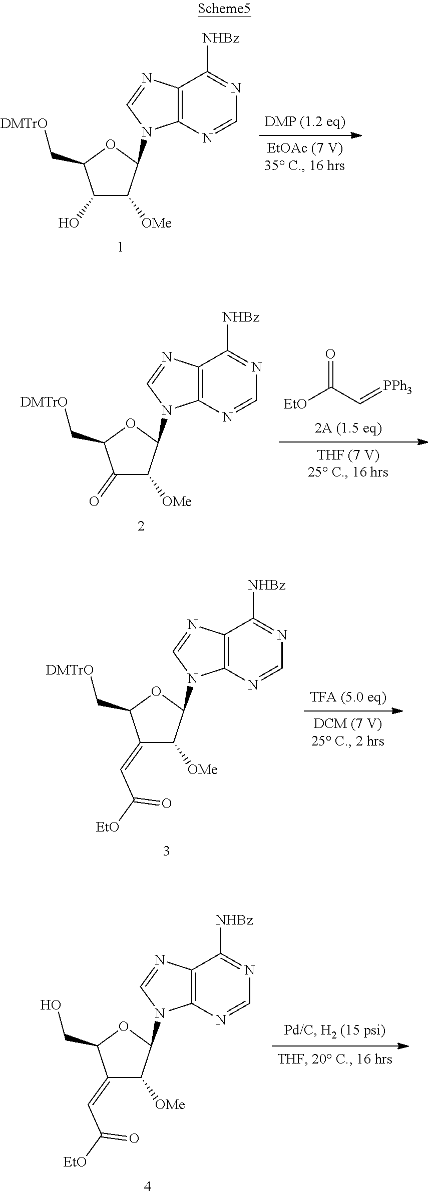

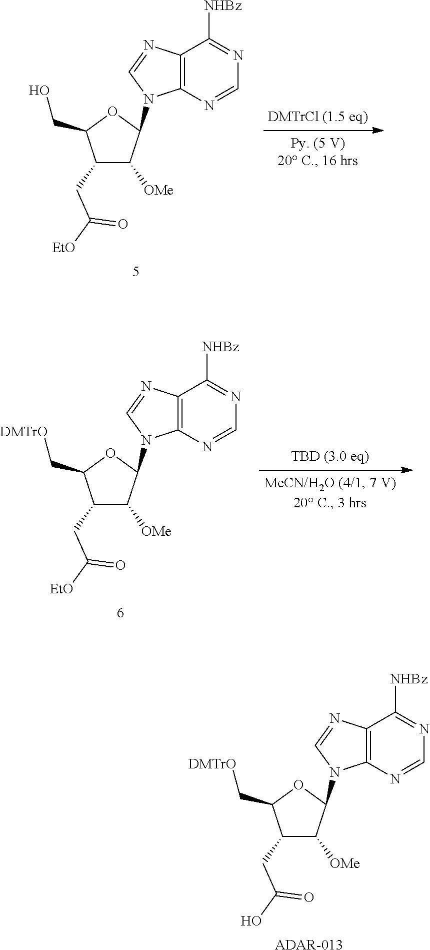

### Experimental

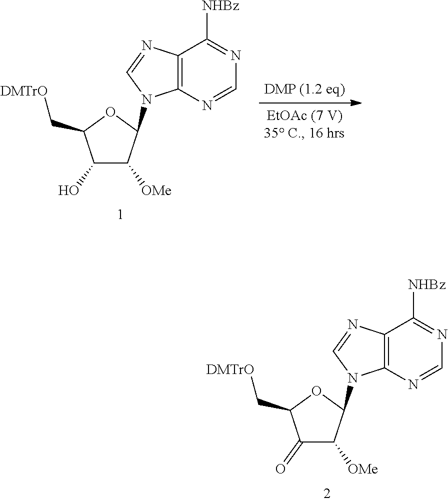

To a solution of compound 1 (200 g, 290 mmol) in EtOAc (1.40 L) was added DMP (148 g, 348 mmol). The mixture was stirred at 35° C. for 16 h. LCMS (ET55793-103-P1A) showed compound 1 was consumed, compound 2 was detected. Using the similar conditions another 2×200 g batches were carried out in parallel, after completing the reaction three batches were combined and filtered and the filter was concentrated under reduced pressure. The mixture was filtered through a flash column. The filtrate was concentrated 'under reduced pressure, to obtain compound 2 (600 g, crude) as yellow solid.

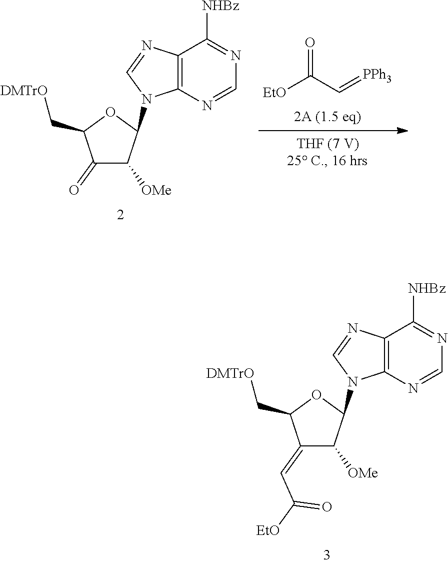

To a solution of compound 2 (200 g, 291 mmol) in THF (1.40 L) was added compound 2A (152 g, 437 mmol). The mixture was stirred at 25° C. for 16 h. LCMS (ET55793-104-P1A3) showed compound 2 was consumed, compound 3 was detected. Using the similar conditions another 2×200 g batches were carried out in parallel, after completing the reaction three batches were combined and concentrate under reduced pressure. The crude was purified by prep-HPLC (MeCN/H₂O) to obtain compound 3 (250 g, 330 mmol, 37.8% yield) as yellow solid.

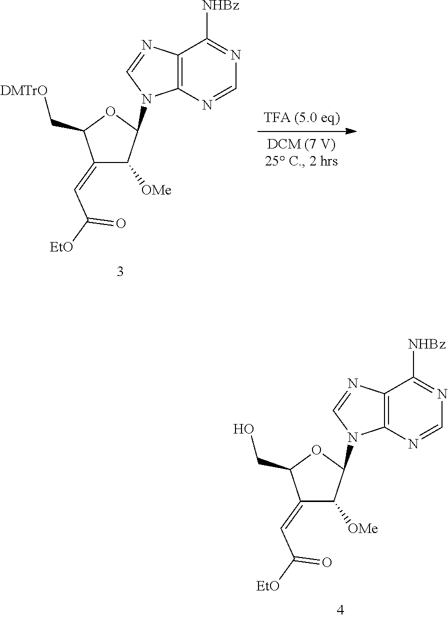

To a solution of compound 3 (310 g, 410 mmol) in DCM (2.17 L) was added TFA (233 g, 2.05 mol, 152 mL) to stir at 25° C. for 2 h. TLC (Petroleum ether:Ethyl acetate=1/1, Petroleum ether/Ethyl acetate=0/1, compound 3 R_{f}=0.70, compound 4 R_{f}=0.50) showed compound 3 was consumed, compound 4 was detected. The reaction was quenched with sat·NaHCO₃ (1.00 L) and the organic layer was washed with brine, dried over Na₂SO₄ and concentrated under reduced pressure. The crude was triturated with MeOH (500 mL) and filtered to give compound 4 (145 g, 319 mmol, 77.9% yield) as a white solid.

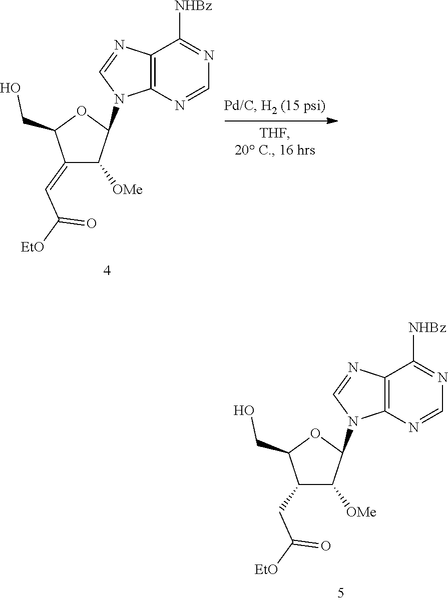

To a round bottom flask added Pd/C (10.0 g, 10% purity) under Argon then charged with THF (350 mL) and added compound 4 (20.0 g, 44.1 mmol). The solution was stirred at 20° C. for 16 h under H₂ (15 psi). LCMS (ET68766-5-P1W1) showed compound 4 was consumed, compound 5 was detected. Using the similar conditions another 4×20 g batches were carried out in parallel, after completing the reaction five batches were combined and filtered and the filter cake was washed with THF (1.00 L). The filtrate was concentrated under reduced pressure to obtain compound 5 (100 g, crude) as a white solid.

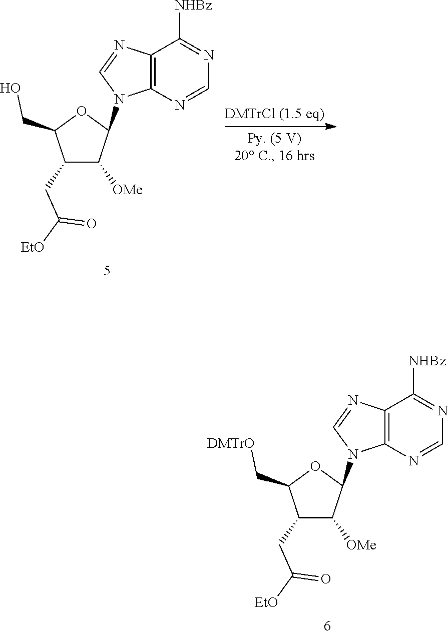

The solution of compound 5 (100 g, 219 mmol) in Py. (500 mL) was added DMTrCl (111 g, 329 mmol) to stir at 20° C. for 16 h. TLC (Petroleum ether:Ethyl acetate=0/1, Petroleum ether/Ethyl acetate=0/1, compound 5 R_{f}=0.30, compound 6 R_{f}=0.60) showed compound 5 was consumed, compound 6 was detected. The reaction was quenched with H₂O (1.50 L) and extracted with EtOAc (1.00 L×2). The combined organic layer was washed with brine, dried over Na₂SO₄ and concentrate under reduced pressure. The crude was purified by column (SiO₂, Petroleum ether/Ethyl acetate=I/O to 0/1) to obtain compound 6 (110 g, 145 mmol, 66.1% yield) as yellow solid.

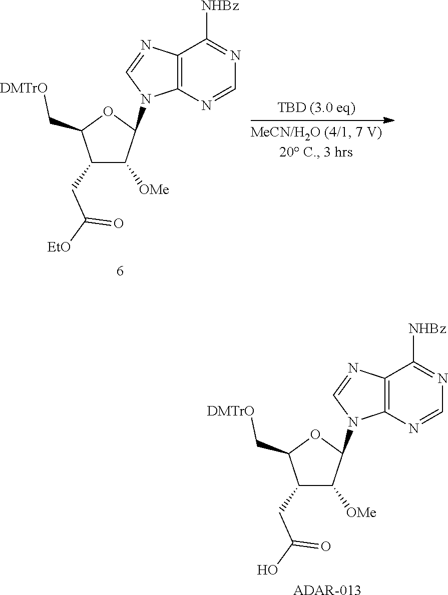

To the solution of compound 6 (200 g, 263 mmol) in MeCN (1.12 L) and H₂O (280 mL) was added TBD (110 g, 791 mmol) and stirred at 20° C. for 3 h. LCMS (ET68766-11-P1A1) showed compound 6 was consumed, ADAR-013 was detected. The reaction was diluted with H₂O (1.50 L) and adjusted to pH<5 with citric acid (10%, 1.00 L). The mixture was extracted with DCM (1.00 L×2). The combined organic layer was washed with brine, dried over Na₂SO₄ and concentrate under reduced pressure. The crude was purified by prep-HPLC to obtain ADAR-013 (102 g, 132 mmol, 50.2% yield, 94.9% purity) as a white solid.

LCMS: m/z, 730 (M⁺+1), ¹H NMR: ET68766-11-P1QW (400 MHz, DMSO-d₆), δ 12.28 (br, 1H), 11.25 (s, 1H), 8.74 (s, 1H), 8.57 (s, 1H), 8.06-8.05 (m, 2H), 7.62-7.55 (m, 3H), 7.37-7.21 (m, 9H), 6.86-6.82 (m, 4H), 6.3 (s, 1H), 4.46-4.45 (d, J=5.6 Hz, 1H), 4.03-4.01 (m, 1H), 3.72 (s, 6H), 3.38 (s, 3H), 3.30-3.25 (m, 2H), 3.01-2.91 (m, 1H), 2.49-2.47 (m, 1H), 2.14-2.07 (m, 1H).

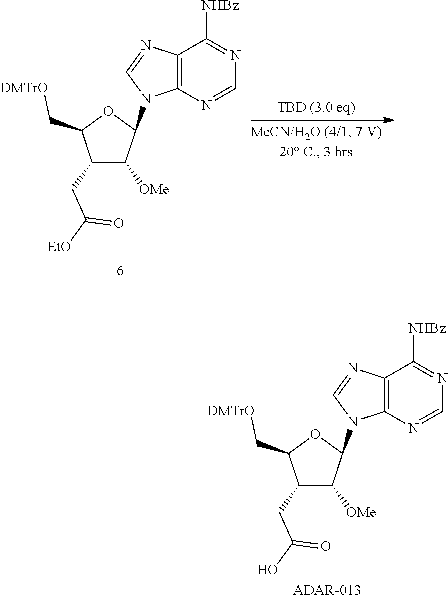

To a solution of compound 1 (240 g, 642 mmol) in Py. (1.20 L) was added DMTrCl (228 g, 674 mmol). The mixture was stirred at 25° C. for 16 hrs. TLC (petroleum ether:ethyl acetate=0:1, compound 2 R_{f}=0.40) showed the reaction was complete. The reaction was diluted with water (1.50 L) and extracted with DCM (800 mL×2). Washed the organic layer with brine (1.00 L). The organic layer was dried with Na₂SO₄ and concentrate in vacuum. Using the similar conditions another 244 g batch wase carried out in parallel, resulting residue from two batches was purified by column chromatography (SiO₂, Petroleum ether/Ethyl acetate=10/1 to 0/1), dried in vacuum to give compound 2 (600 g, 816 mmol, 63.5% yield, 92.0% purity) as white solid.

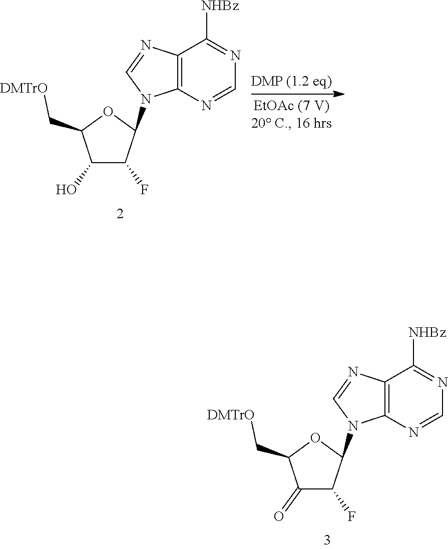

DMP (150 g, 355 mmol, 109 mL) was added to a solution of compound 2 (200 g, 295 mmol) in EtOAc (1400 mL). The mixture was stirred at 20° C. for 16 hrs. TLC (petroleum ether:ethyl acetate=0:1, compound 3 R_{f}=0.45) showed the reaction was complete. Added sat. NaHCO₃ (1000 mL) to the mixture and stirred at 20° C. for 1 hr, filtered and separated the organic layer, and extracted the aq. layer with EtOAc (500×2 mL), washed with brine (1000 mL). Using the similar conditions another 150 g batch was carried out in parallel. Combined the two batches, dried over Na₂SO₄, and concentrate in vacuum to give compound 3 (400 g, crude) as yellow oil.

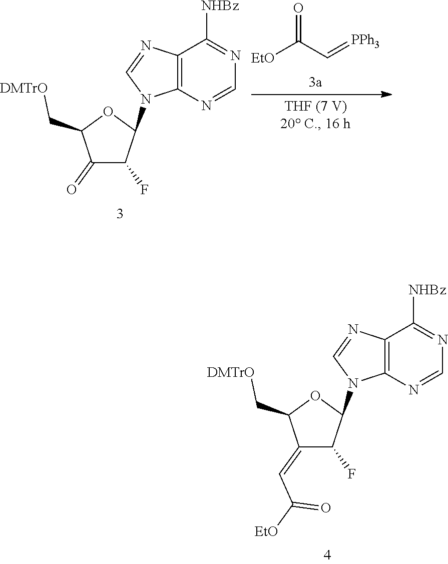

Compound 3a (155 g, 445 mmol) was added to a solution of compound 3 (200 g, 296 mmol) in THF (1.40 L). The mixture was stirred at 20° C. for 16 hrs. LCMS (ET60061-129-P1C₁, product RT=2.415 min) showed the reaction was completed. Using the similar conditions another 200 g batch was carried out in parallel, after completing the reaction two batches were combined and concentrate under reduced pressure. The crude product was purified by reverse-phase HPLC (neutral condition) to give compound 4 (310 g, 325 mmol, 54.7% yield) as yellow oil.

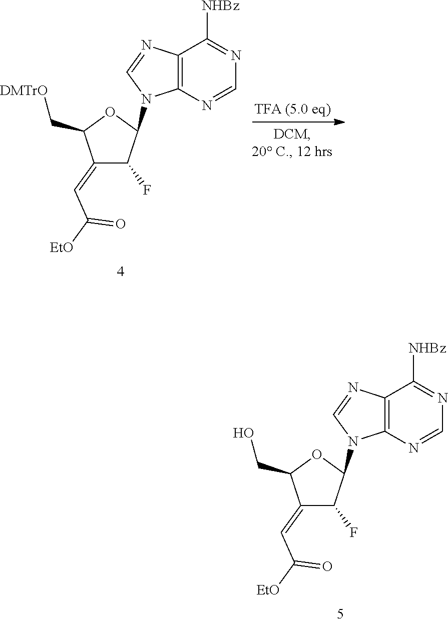

A mixture of compound 4 (310 g, 416 mmol,) and TFA (237 g, 2.08 mol, 154 mL) in DCM (1.50 L) was stirred at 20° C. for 12 hrs. TLC (petroleum ether:ethyl acetate=0:1, compound 5 R_{f}=0.20) showed the reaction was complete. The mixture was concentrated under reduced pressure The residue was purified by column chromatography on silica gel (100-200 mesh silica gel) eluted with petroleum ether:ethyl acetate (100: 1-0:1) to give compound 5 (110 g, 196 mmol, 47.2% yield) as a yellow oil.

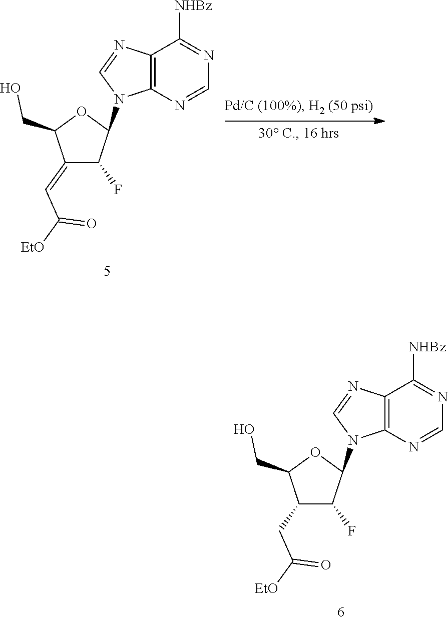

Compound 5 (10.0 g, 22.6 mmol) was added to a solution of Pd/C (10.0 g, 10% purity) in THF (500 mL). The mixture was stirred at 30° C. for 16 hrs under H₂ atmosphere (50 psi). LCMS (ET60061-161-P1B2, product RT=0.599 min) showed the reaction was completed. Using the similar conditions another 4×10 g batches were carried out in parallel, after completing the reaction five batches were combined, filter and concentrate in vacuum. The residue was purified by column chromatography (SiO₂, dichloromethane:methanol=100/1 to 0/1) to give compound 6 (36.0 g, 76.3 mmol, 67.3% yield) as yellow oil.

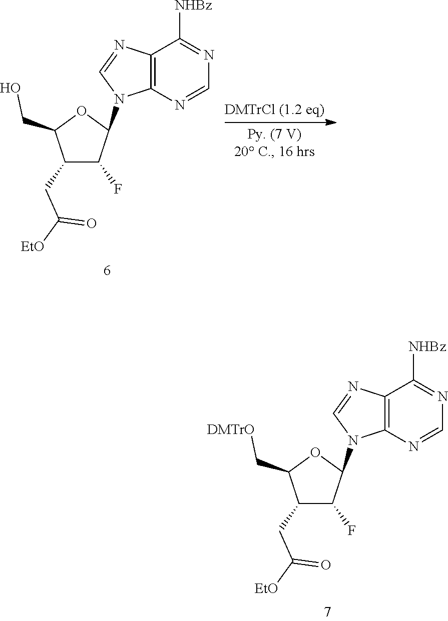

A mixture of compound 6 (80.0 g, 180 mmol) and DMTCl (73.3 g, 216 mmol) in Pyridine. (400 mL) was degassed and purged with N₂ for 3 times, and then the mixture was stirred at 20° C. for 16 hrs under N₂ atmosphere. TLC (petroleum ether:ethyl acetate=0:1, compound 7 R_{f}=0.40) showed the reaction was complete. The reaction was quenched with H2O (1.50 L) and extracted with EtOAc (1.00 L×2). The combined organic layer was washed with brine, dried over Na₂SO₄, and concentrate under reduced pressure. The residue was purified by column chromatography (SiO₂, Petroleum ether/Ethyl acetate=100/1 to 0/1) to give compound 7 (80.0 g, 100 mmol, 55.8% yield, 94.9% purity) as yellow solid.

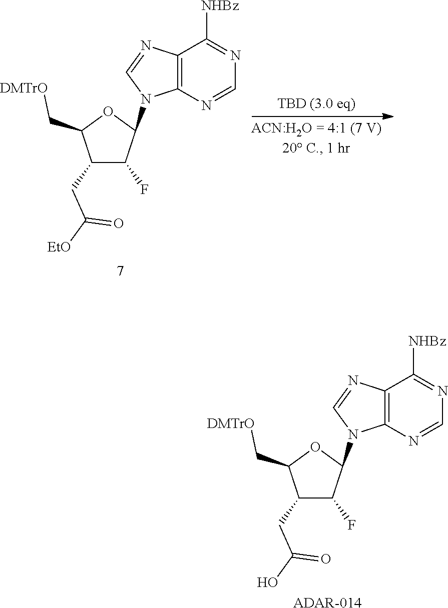

A solution of compound 7 (80.0 g, 107 mmol) in ACN (448 mL) and H₂O (112 mL) was added TBD (44.7 g, 321 mmol). The mixture was stirred at 20° C. for 1 hr. TLC (petroleum ether:ethyl acetate=0:1, ADAR-014 R_{f}=0.20) showed the reaction was completed. The mixture was diluted with DCM (600 mL) and washed with citric acid (10%, pH=6). The organic layer was dried over Na₂SO₄ and concentrated under reduced pressure to give a residue. The crude product was purified by reversed-phase HPLC (neutral condition) to give ADAR-014 (58.0 g, 75.3% yield) as yellow solid. LCMS: m/z, 718 (M⁺+1), ¹H NMR: (400 MHz, DMSO-d₆), δ 12.44 (brs, 1H), 11.25 (s, 1H), 8.71 (s, 1H), 8.56 (s, 1H), 8.04-8.06 (m, 2H), 7.53-7.66 (m, 3H), 7.16-7.33 (m, 7H), 6.79-6.85 (m, 4H), 6.43 (d, J=20.8 Hz, 1H), 5.77-5.91 (m, 1H), 4.12-4.15 (m, 1H), 3.72 (s, 6H), 3.27-3.41 (m, 3H), 2.23-2.28 (m, 1H).

### Synthesis of ADAR-016

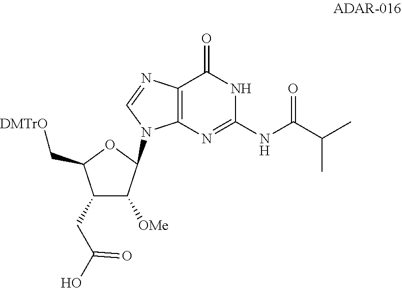

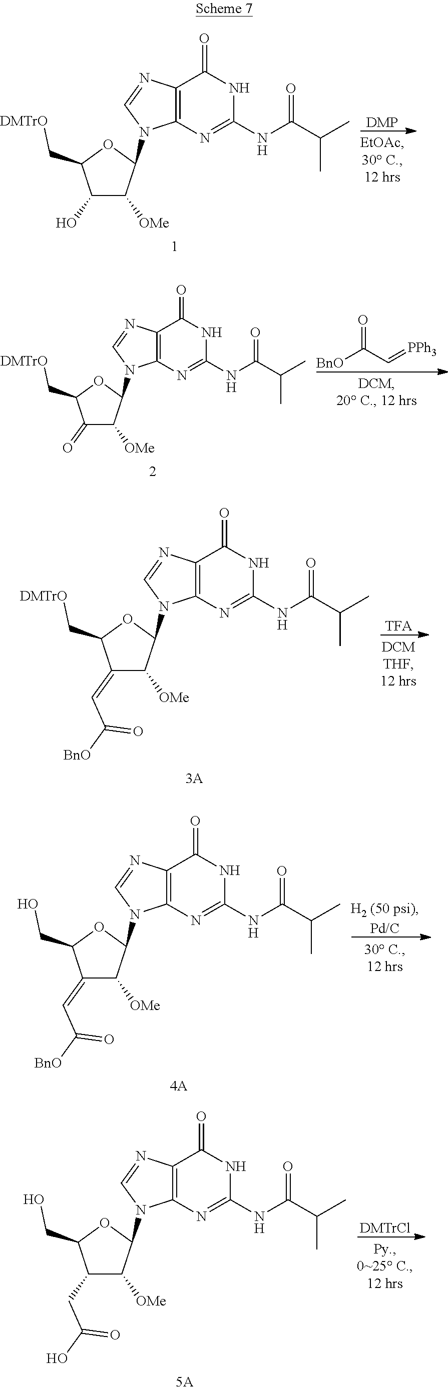

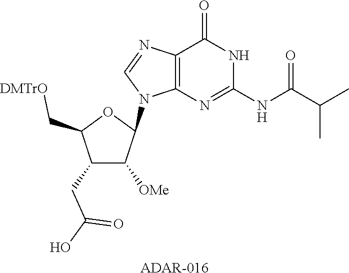

### Experimental

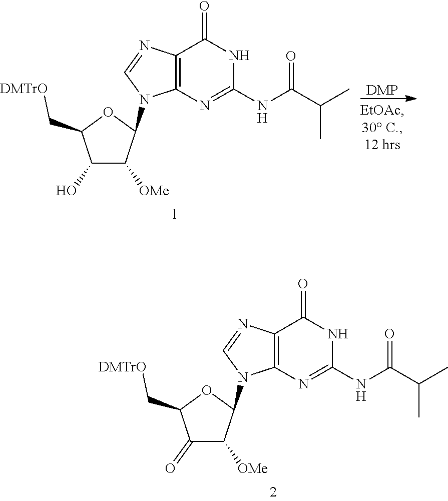

To a stirred suspension of compound 1 (100 g, 149 mmol, 1.00 eq) in EtOAc (2000 mL) was added DMP (95.0 g, 223 mmol, 69.3 mL, 1.50 eq). The mixture was stirred at 30° C. for 12 hrs. TLC (petroleum ether/ethyl acetate=5/1, R_{f}=0.25) showed the reaction was complete. Using the similar conditions another 3×100 g batches were carried out in parallel, after completing the reaction four batches were combined and filtered and the filter was concentrated under reduced pressure resulting residue was purified by column directly (SiO₂, using Petroleum ether/Ethyl acetate=2/1 to 0/1) to obtain compound 2 (400 g, crude) as a white solid, used directly for next step.

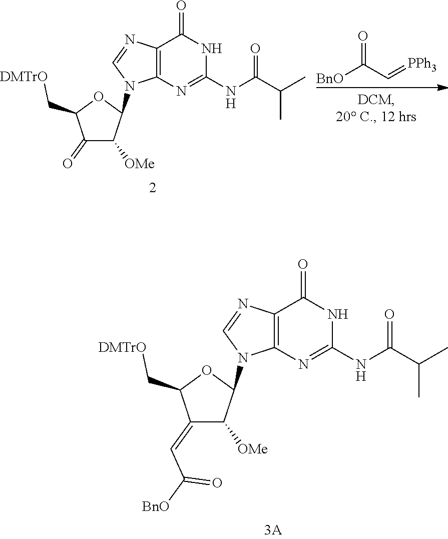

To a solution of compound 2 (50.0 g, 74.8 mmol, 1.00 eq) in DCM (1300 mL) was added dropwise a solution of benzyl 2-(triphenyl-phosphanylidene)acetate (46.1 g, 112 mmol, 1.50 eq) in DCM (200 mL) at 0° C. The mixture was stirred at 25° C. for 12 hrs. LCMS (ET58200-139-P1L1, product: RT=2.452 min,) showed the reaction was completed. Using the similar conditions another 3×50 g batches were carried out in parallel, after completing the reaction four batches were combined and filtered and the filtrate was concentrated under reduced pressure to give a residue. Then it was purified by prep-HPLC to obtain compound 3A (75.0 g, 93.7 mmol, 31.3% yield) as an orange solid with 92.9% LCMS purity. ¹H NMR: 400 MHz, CD₃OD. δ 8.16 (s, 1H), 7.31-7.17 (m, 15H), 6.78-6.76 (m, 4H), 6.10-6.09 (m, 1H), 5.91 (d, J=8.0 Hz, 1H), 5.42-5.41 (m, 1H), 5.30-5.29 (m, 1H), 5.11-4.99 (m, 2H), 3.72 (s, 6H), 3.36 (s, 3H), 3.31-3.30 (m, 2H), 3.26-3.23 (m, 1H), 2.61-2.58 (m, 1H), 1.18-1.15 (m, 6H).

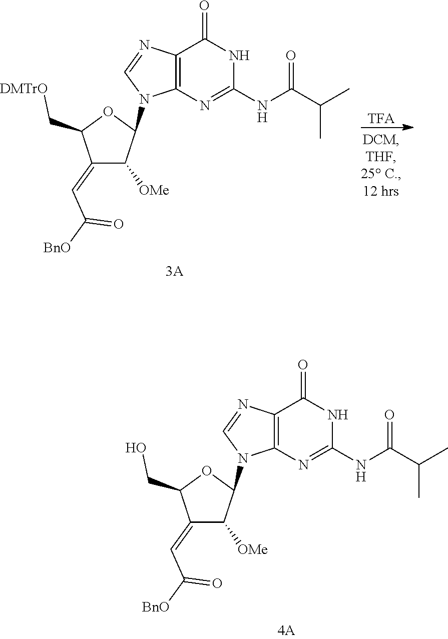

To a solution of compound 3A (100 g, 125 mmol, 1.00 eq) in DCM (700 mL) was added TFA (107 g, 942 mmol, 70.0 mL, 7.54 eq) at 25° C. After addition, the resulting mixture was stirred at 25° C. for 12 hrs. LCMS (ET58200-142-P1L1, product: Rₜ=1.766 min,) showed the reaction was completed. Using the similar conditions another 100 g batch was carried out in parallel, after completing the reaction two batches were combined and concentrate in vacuum resulting residue was purified by column (SiO₂, using Dichloromethane/Methanol=50/1 to 2/1). Obtain compound 4A (124 g, 88.9% yield, 89.2% purity) as orange solid.

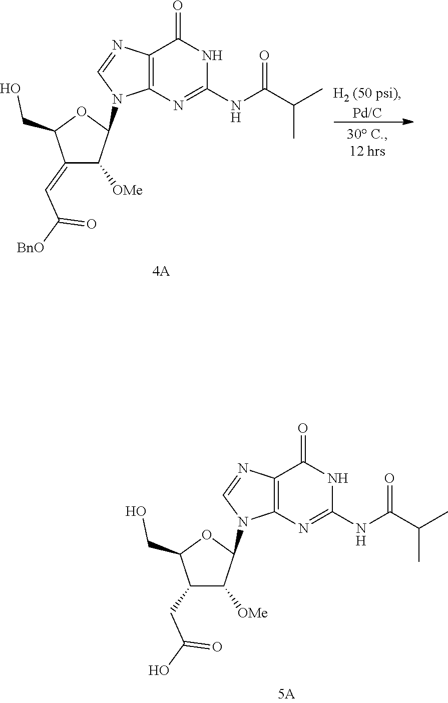 [SMILES: CO[C@@H]1/C(=C\C(=O)OCc2ccccc2)[C@@H](CO)O[C@H]1n1cnc2c(=O)[nH]c(NC(=O)C(C)C)nc21 ; "H2 (50 psi),Pd/C" ; CO[C@@H]1[C@H](CC(=O)O)[C@@H](CO)O[C@H]1n1cnc2c(=O)[nH]c(NC(=O)C(C)C)nc21]

To a solution of compound 4A (31.0 g, 62.3 mmol, 1.00 eq) in THF (210 mL) was added Pd/C (31.0 g, 10% purity) under N₂ atmosphere. The suspension was degassed and purged with H₂ for 3 times. The mixture was stirred under H₂ (50 Psi) at 30° C. for 12 hrs. LCMS (ET58200-143-P1L1) showed the reaction was completed. Using the similar conditions another 3×31 g batches were carried out in parallel, after completing the reaction four batches were combined and filtered, the filter cake was washed with THF (200 mL×3). The filtrate was concentrated under reduced pressure to give crude product. Then the crude product was triturated with MTBE (1.0 L) at 20° C. for 2 hrs, then filtered, to obtain compound 5A (95.0 g, 84.7% yield, 91.7% purity) as orange solid.

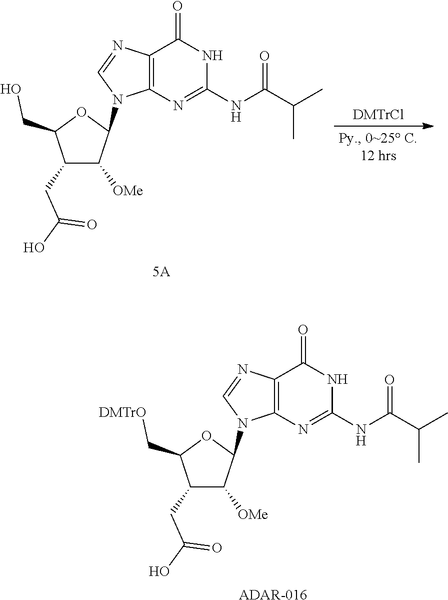

To a solution of compound 5A (95.0 g, 208 mmol, 1.00 eq) in pyridine (350 mL) was added DMTrCl (106 g, 313 mmol, 1.50 eq) at 0° C. under the N₂ protection. The reaction was stirred at 25° C. for 12 hrs. TLC (Dichloromethane:Methanol=5:1) indicated starting material was consumed completely and one new spot formed (R_{f}=0.5). The reaction was clean according to TLC. Reaction mixture was concentrate in vacuum to remove pyridine, the crude was purified by prep-MPLC, to obtain ADAR-016 (106 g, 148 mmol, 64% yield, 99.7% purity) as a white solid with 99.7%. LCMS: m/z, 712 (M⁺+1). ¹H NMR: (400 MHz, DMSO-d₆), δ 12.27 (brs, 1H), 12.13 (s, 1H), 11.50 (s, 1H), 8.06 (s, 1H), 7.34-7.26 (m, 2H), 7.24-7.20 (m, 7H), 6.85-6.81 (m, 4H), 6.01 (s, 1H), 4.23 (d, J=6.0 Hz, 1H), 4.00-3.98 (m, 1H), 3.72 (s, 6H), 3.33 (s, 3H), 3.27-3.25 (m, 2H), 2.82-2.78 (m, 2H), 2.49-2.42 (m, 1H), 2.10-2.09 (m, 1H), 2.09-2.07 (m, 1H), 1.14-1.11 (m, 6H).

### Synthesis of ADARx-017

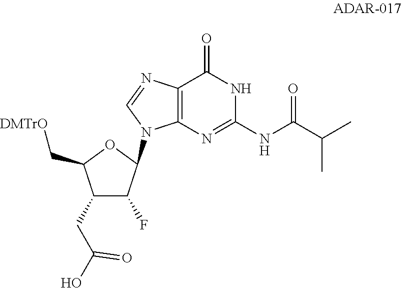

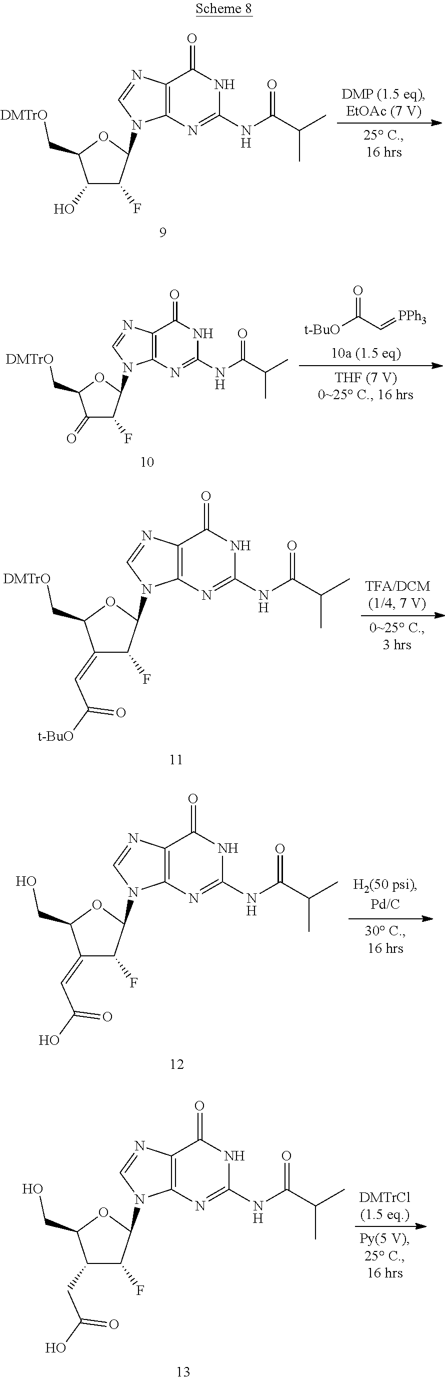

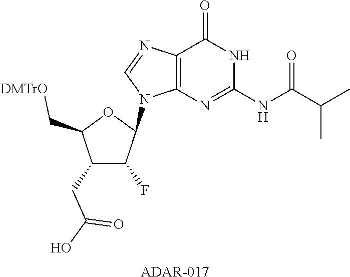

### Experimental

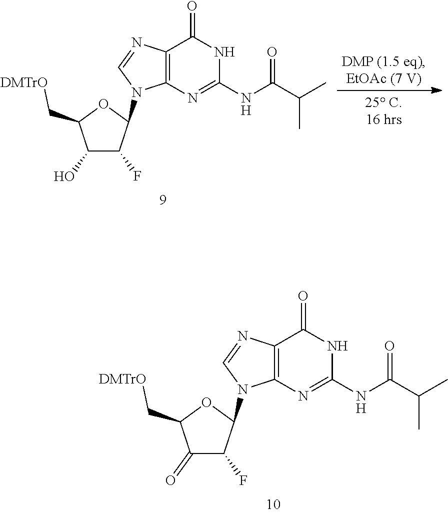

A mixture of compound 9 (200 g, 304 mmol), DMP (193 g, 456 mmol) in EtOAc (1.40 L) was degassed and purged with N₂ for 3 times, and then the mixture was stirred at 25° C. for 16 hrs under N₂ atmosphere. LCMS (ET61738-29-P1A) showed compound 9 was consumed, compound 10 was detected. Using the similar conditions another 200 g batch was carried out in parallel, after completing the reaction two batches were combined and quenched by sat. NaHCO₃ (1.00 L) and filtered. The aqueous phase was extracted with EtOAc (1.5 L×2). The combined organic layer was washed with brine, dried over Na₂SO₄, and concentrate under reduced pressure, to give compound 10 (400 g, crude) as yellow solid.

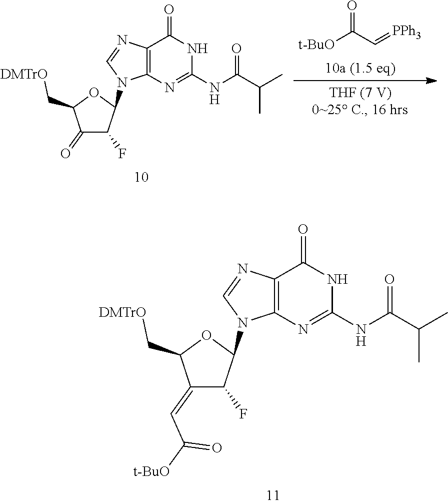

To a solution of compound 10 (200 g, 305 mmol) in THF (1.40 L) was added compound 10a (172 g, 457 mmol) in portions under 0° C. The mixture was stirred at 25° C. for 16 hrs. LCMS (ET61738-32-P1A) showed compound 10 was consumed, compound 11 was detected. Using the similar conditions another 200 g batch was carried out in parallel, after completing the reaction two batches were combine, filtered and the filter cake was washed with EtOAc (800 mL). The filtrate was concentrate under reduced pressure. The crude was purified by prep-HPLC (H₂O/MeCN) to obtain compound 11 (157 g, 208 mmol, 34.1% yield) as yellow solid.

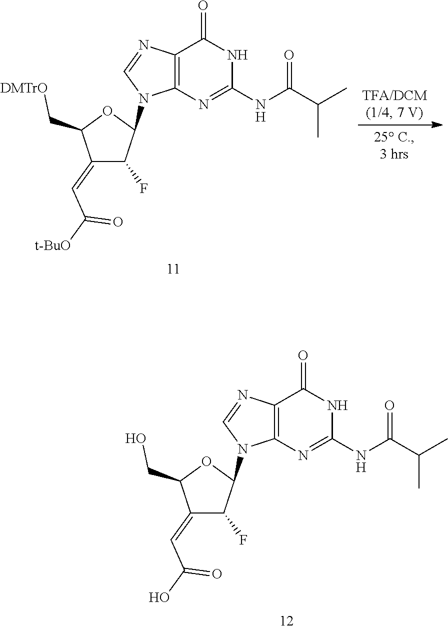

A mixture of compound 11 (157 g, 208 mmol), TFA (220 mL) in DCM (880 mL) was degassed and purged with N₂ for 3 times, and then the mixture was stirred at 25° C. for 3 hrs under N₂ atmosphere. LCMS (ET61738-34-P1A) showed compound 11 was consumed, compound 12 was detected. The reaction was concentrated under reduced pressure. The crude was purified by column chromatography (SiO₂, Petroleum ether/Ethyl acetate=I/O to 0/1) to obtain compound 12 (48.0 g, 121 mmol, 58.3% yield) as yellow solid.

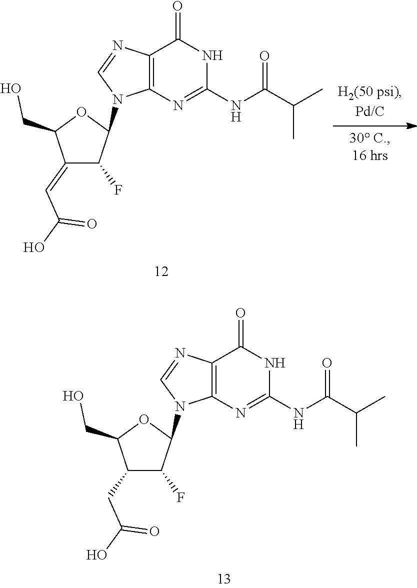 [SMILES: CC(C)C(=O)Nc1nc2c(ncn2[C@@H]2O[C@H](CO)/C(=C/C(=O)O)[C@H]2F)c(=O)[nH]1 ; "H2(50 psi),Pd/C" ; CC(C)C(=O)Nc1nc2c(ncn2[C@@H]2O[C@H](CO)[C@@H](CC(=O)O)[C@H]2F)c(=O)[nH]1]

To a solution of compound 12 (10.0 g, 25.2 mmol,) in THF (70.0 mL) was added Pd/C (10.0 g, 25.2 mmol, 10% purity) under N₂ atmosphere. The suspension was degassed and purged with H₂ for 3 times. The mixture was stirred under H₂ (50 psi) at 30° C. for 16 hrs. LCMS (ET61738-41-P1A6) showed compound 12 was consumed, compound 13 was detected. Using the similar conditions another 4×10 g batches were carried out in parallel, after completing the reaction five batches were combined and filtered, and the filter cake was washed with THF (1.00 L). The filtrate was concentrated under reduced pressure. To obtain compound 13 (50.0 g, 125 mmol, 82.9% yield) as a white solid. LCMS: m/z, 398 (M⁺+1).

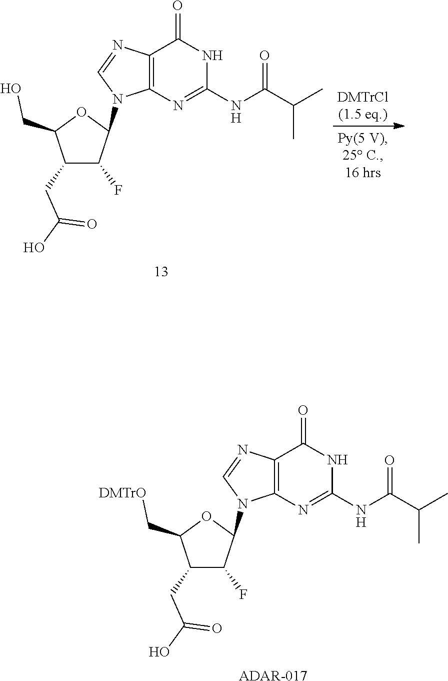

To a solution of compound 13 (50.0 g, 125 mmol) in Pyridine. (250 mL) was added DMTrCl (63.9 g, 188 mmol). The mixture was stirred at 25° C. for 16 hrs. TLC (petroleum ether:ethyl acetate=0:1, ADAR-017 R_{f}=0.20) showed compound 13 was consumed, ADAR-017 was detected. The reaction was quenched with H₂O (1.00 L) and extracted with EtOAc (800 mL×2). The combined organic layer was washed with brine (500 mL), dried with Na₂SO₄ and concentrate under reduced pressure. The crude was purified by prep-HPLC (neutral condition, column: Welch Xtimate C18 250*100 mm #10 um; mobile phase: [water (NH₄HCO₃)-ACN]; B %: 25%-45%, 20 min) to obtain ADAR-017 (62.9 g, 95.2% purity) as a white solid. LCMS: m/z, 700 (M⁺+1). ¹H NMR: (400 MHz, DMSO-d₆), δ 8.04 (s, 1H), 7.35-7.21 (m, 9H), 6.85-6.81 (m, 4H), 6.21-6.17 (d, J=18.0 Hz, 1H), 5.63-5.49 (d, J=55.6 Hz, 1H), 4.11-4.09 (m, 1H), 3.72 (s, 6H), 3.41-3.29 (m, 2H), 3.02-2.93 (m, 1H), 2.51-2.39 (m, 1H), 2.81-2.77 (m, 1H), 2.27-2.19 (m, 1H), 1.13-1.11 (d, J=6.8 Hz, 6H).

### Example 6: Synthesis of Amine Monomers Experimental

### Synthesis of ADARx-1a

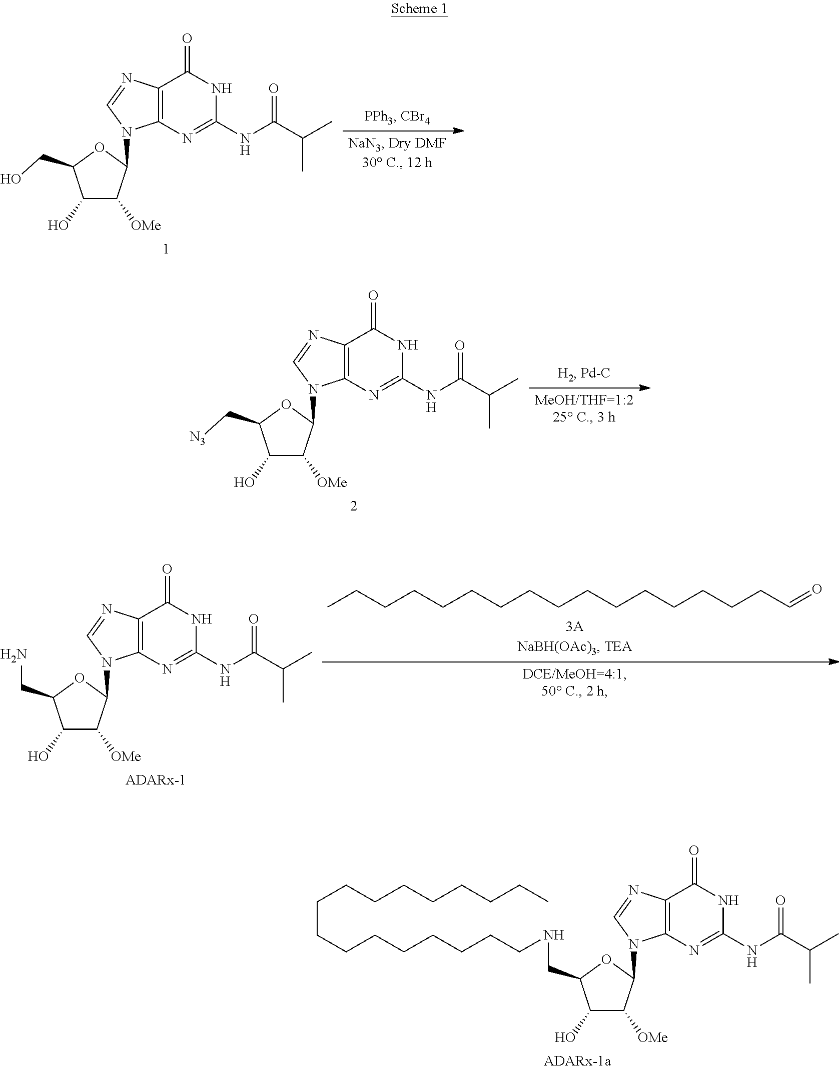

### N-(9-((2R,3R,4R,5R)-5-(azidomethyl)-4-hydroxy-3-methoxytetrahydrofuran-2-yl)-6-oxo-6,9-dihydro-1H-purin-2-yl)isobutyramide (2)

To a solution of N-[9-[(2R,3R,4R,5R)-4-hydroxy-5-(hydroxymethyl)-3-methoxy-tetrahydrofuran-2-yl]-6-oxo-1H-purin-2-yl]-2-methyl-propanamide (15 g, 40.83 mmol, 1 eq) in DMF (300 mL) was added PPh₃ (21.42 g, 81.66 mmol, 2 eq) and NaN₃ (15.93 g, 244.99 mmol, 6 eq). Then CBr₄ (27.08 g, 81.66 mmol, 2 eq) was added in portions at 0° C. The mixture was stirred at 30° C. for 12 hours. The reaction mixture was diluted with ethyl acetate (300 mL) and filtered to give a filtrate, then concentrated under reduced pressure to give a residue. The residue was purified by flash silica gel chromatography (ISCO®; 220 g SepaFlash® Silica Flash Column, Eluent of 0-10% MeOH/DCM gradient @100 mL/min) to afford N-[9-[(2R,3R,4R,5R)-5-(azidomethyl)-4-hydroxy-3-methoxy-tetrahydrofuran-2-yl]-6-oxo-1H-purin-2-yl]-2-methyl-propanamide (2) (16 g, 40.78 mmol, 99.87% yield) as a white solid. MS ES⁺:393.1, ¹H NMR (400 MHz, DMSO-d₆) δ 12.10 (br s, 1H), 11.62 (br s, 1H), 8.29 (s, 1H), 5.93 (d, J=6.1 Hz, 1H), 5.46 (d, J=5.1 Hz, 1H), 4.39 (t, J=5.5 Hz, 1H), 4.33-4.25 (m, 1H), 4.07-4.01 (m, 1H), 3.71-3.62 (m, 1H), 3.61-3.52 (m, 1H), 3.35 (s, 3H), 2.84-2.69 (m, 1H), 1.13 (d, J=6.8 Hz, 6H).

### N-(9-((2R,3R,4R,5R)-5-(aminomethyl)-4-hydroxy-3-methoxytetrahydrofuran-2-yl)-6-oxo-6,9-dihydro-1H-purin-2-yl)isobutyramide (ADARx-1)

To a solution of N-[9-[(2R,3R,4R,5R)-5-(azidomethyl)-4-hydroxy-3-methoxy-tetrahydrofuran-2-yl]-6-oxo-1H-purin-2-yl]-2-methyl-propanamide (16 g, 40.78 mmol, 1 eq) in MeOH:THF=1:2 (300 mL) was added Pd/C (3.4 g, 40.78 mmol, 10% purity, 1 eq) under N₂. The suspension was degassed under vacuum and purged with H₂ several times. The mixture was stirred under H₂ (82.20 mg, 40.78 mmol, 1 eq) (15 psi) at 25° C. for 3 hours. The reaction mixture was filtered and washed with MeOH, then concentrated under reduced pressure. The crude product was triturated with EA (300 mL) at 25° C. for 12 hours to afford N-[9-[(2R,3R,4R,5R)-5-(aminomethyl)-4-hydroxy-3-methoxy-tetrahydrofuran-2-yl]-6-oxo-1H-purin-2-yl]-2-methyl-propanamide (ADARx-1) (12 g, 32.75 mmol, 80.32% yield) as a off-white solid. MS ES⁺:367.1, ¹H NMR (400 MHz, DMSO-d₆) δ 8.32 (s, 1H), 5.89 (d, J=6.3 Hz, 1H), 4.35-4.30 (m, 1H), 4.29-4.25 (m, 1H), 3.89-3.83 (m, 1H), 3.39-3.25 (m, 3H), 2.91-2.68 (m, 3H), 1.12 (d, J=6.9 Hz, 6H)

### N-(9-((2R,3R,4R,5R)-5-((heptadecylamino)methyl)-4-hydroxy-3-methoxytetrahydrofuran-2-yl)-6-oxo-6,9-dihydro-1H-purin-2-yl)isobutyramide (ADARx-1a)

To a solution of N-[9-[(2R,3R,4R,5R)-5-(aminomethyl)-4-hydroxy-3-methoxy-tetrahydrofuran-2-yl]-6-oxo-1H-purin-2-yl]-2-methyl-propanamide (3 g, 8.19 mmol, 1 eq) and heptadecanal (2.08 g, 8.19 mmol, 1 eq) in DCE:MeOH=4:1 (60 mL) was added TEA (1.66 g, 16.38 mmol, 2.28 mL, 2 eq) and 4A MS (1.2 g). After stirred at 50° C. for 30 min, NaBH(OAc)₃ (6.94 g, 32.75 mmol, 4 eq) was added in portions. The mixture was stirred at 50° C. for 2 hours. The reaction mixture was filtered and concentrated under reduced pressure. The residue was diluted with sat. NaHCO₃ (aq.) (500 mL) and extracted with DCM 500 mL (100 mL*5). The combined organic layers were concentrated under reduced pressure to give a residue. The residue was purified by flash silica gel chromatography (ISCO®; 40 g SepaFlash® Silica Flash Column, Eluent of 0-5% Ethyl acetate/Petroleum ethergradient @100 mL/min) to afford N-[9-[(2R,3R,4R,5R)-5-[(heptadecylamino)methyl]-4-hydroxy-3-methoxy-tetrahydrofuran-2-yl]-6-oxo-1H-purin-2-yl]-2-methyl-propanamide(ADARx-1a) (2.2 g, 3.47 mmol, 42.40% yield, 95.455% purity) as a yellow solid. MS ES+: 605.5, ¹H NMR (400 MHz, DMSO-d₆) δ 8.28 (s, 1H), 5.88 (d, J=5.5 Hz, 1H), 5.48-5.03 (m, 1H), 4.32-4.27 (m, 2H), 3.98-3.92 (m, 1H), 3.33 (s, 3H), 2.82-2.71 (m, 3H), 2.53 (br s, 2H), 1.46-1.33 (m, 2H), 1.22 (br d, J=3.6 Hz, 28H), 1.12 (d, J=6.9 Hz, 6H), 0.90-0.80 (m, 3H).

### Synthesis of ADARx-2 and ADARx-2a

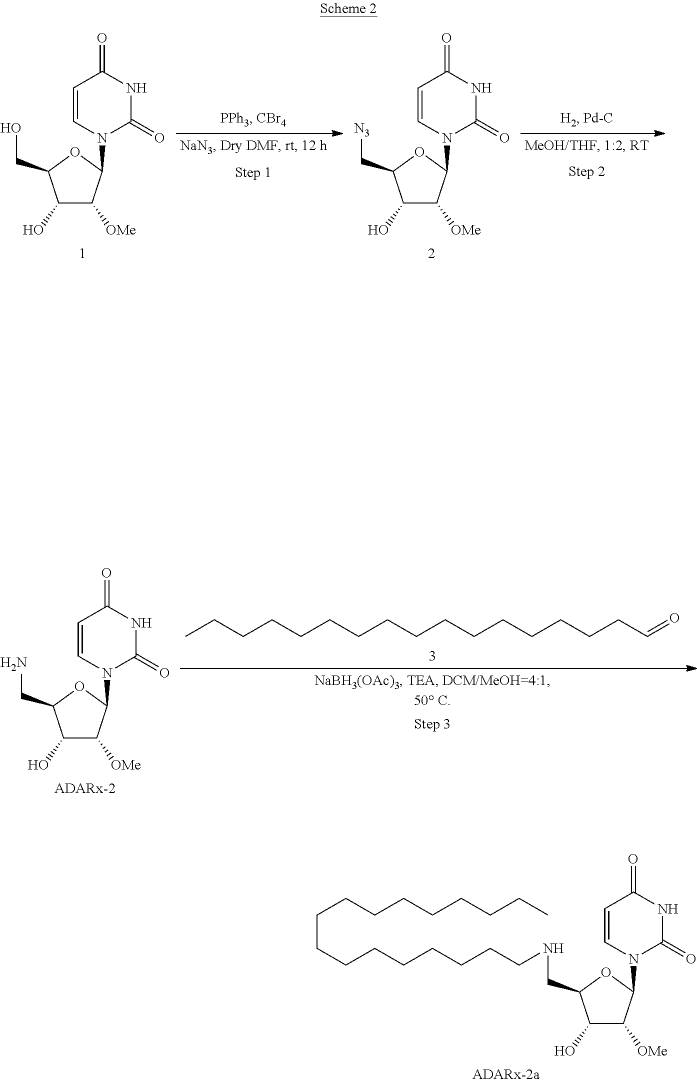

Step 1: To a solution of 1-[(2R,3R,4R,5R)-4-hydroxy-5-(hydroxymethyl)-3-methoxy-tetrahydrofuran-2-yl]pyrimidine-2,4-dione 1 (15 g, 58.09 mmol, 1 eq) in DMF (300 mL) was added PPh₃ (30.47 g, 116.18 mmol, 2 eq), NaN₃ (22.66 g, 348.53 mmol, 6 eq) and CBr₄ (38.53 g, 116.18 mmol, 2 eq) sequently. The mixture was stirred at 30° C. for 12 hours. LC-MS showed the start material was consumed completely and one main peak with desired m/z was detected. The reaction mixture was diluted with EtOAc (200 mL) and filtered to give a filtrate, then concentrated under reduced pressure to give a residue. The residue was purified by flash silica gel chromatography (ISCO®; 330 g SepaFlash® Silica Flash Column, Eluent of Ethyl acetate/Petroleum ethergradient 0-50% 30 min, 50-50% 180 min @100 mL/min) to give the title compound 2 (19.47 g, crude) as a pale-yellow foam. MS ES⁺: 284.0, ¹H NMR (400 MHz, DMSO-d₆) δ=11.41 (br s, 1H), 7.70 (d, J=8.1 Hz, 1H), 5.83 (d, J=4.8 Hz, 1H), 5.68 (dd, J=2.1, 8.0 Hz, 1H), 5.36 (d, J=6.3 Hz, 1H), 3.96-3.83 (m, 2H), 3.60 (d, J=4.8 Hz, 2H), 3.36 (s, 3H).

Step 2: A mixture of 1-[(2R,3R,4R,5R)-5-(azidomethyl)-4-hydroxy-3-methoxy-tetrahydrofuran-2-yl]pyrimidine-2,4-dione 2 (19.47 g, 68.74 mmol, 1 eq), Pd/C (4 g, 10% purity) in MeOH (200 mL) and THF (400 mL) was degassed and purged with H₂ for 3 times, and then the mixture was stirred at 25° C. for 2 hours under H₂ (15PSI). LCMS showed the desired compound was formed. The combined organic layers were filtered and concentrated under reduced pressure to give a residue. The residue was purified by flash silica gel chromatography (ISCO®; 120 g SepaFlash® Silica Flash Column, Eluent of 0-40% MeOH/DCM @100 mL/min) to give 1-((2R,3R,4R,5R)-5-(aminomethyl)-4-hydroxy-3-methoxytetrahydrofuran-2-yl)pyrimidine-2,4(1H,3H)-dione ADARx-2 (12.3 g, 47.81 mmol, 69.56% yield) as a white solid. MS ES⁺: 258.0, ¹H NMR (400 MHz, DMSO-d₆) δ=7.95 (d, J=8.1 Hz, 1H), 5.82 (d, J=5.1 Hz, 1H), 5.63 (d, J=8.1 Hz, 1H), 5.24-4.96 (m, 1H), 4.07 (br s, 1H), 3.82 (br t, J=5.2 Hz, 1H), 3.75 (q, J=4.3 Hz, 1H), 3.34-3.33 (m, 3H), 2.84-2.69 (m, 2H).

Step 3: To a solution of 1-[(2R,3R,4R,5R)-5-(aminomethyl)-4-hydroxy-3-methoxy-tetrahydrofuran-2-yl]pyrimidine-2,4-dione ADARx-2 (4.9 g, 19.05 mmol, 1 eq) and heptadecanal (4.85 g, 19.05 mmol, 1 eq) in DCE:MeOH=4:1 (100 mL) was added 4A MS (2 g, 19.05 mmol, 1 eq) and TEA (3.85 g, 38.10 mmol, 5.30 mL, 2 eq), after stirring at 50° C. for 1 hour, NaBH(OAc)₃ (16.15 g, 76.19 mmol, 4 eq) was added. The mixture was stirred at 50° C. for 12 hours. LC-MS and TLC (DCM:MeOH=10:1, R_{f}=0.30) showed the start material was consumed completely and one main peak with desired m/z was detected. The reaction mixture was filtered with MeOH and concentrated under reduced pressure to give a residue. The residue was diluted with aq. NaHCO₃ (800 ml) and extracted with CHCl₃ (500 mL×3). The combined organic layers were dried over Na₂SO₄, filtered and concentrated under reduced pressure to give a residue which was purified by flash silica gel chromatography (ISCO®; 120 g SepaFlash® Silica Flash Column, Eluent of 0-6% MeOH/DCM @100 mL/min) to give the title compound ADARx-2a (3.1 g, 6.25 mmol, 32.83% yield) as a yellow solid. MS ES⁺: 496.6, ¹H NMR (400 MHz, DMSO-d₆) δ=7.92 (d, J=8.1 Hz, 1H), 5.80 (d, J=4.9 Hz, 1H), 5.60 (d, J=8.1 Hz, 1H), 5.25-5.02 (m, 1H), 4.09-3.98 (m, 1H), 3.87-3.78 (m, 2H), 3.35 (s, 3H), 2.78-2.66 (m, 2H), 2.52-2.51 (m, 2H), 1.42-1.35 (m, 2H), 1.23 (s, 28H), 0.91-0.78 (m, 3H).

### Synthesis of ADARx-3 and ADARx-3a

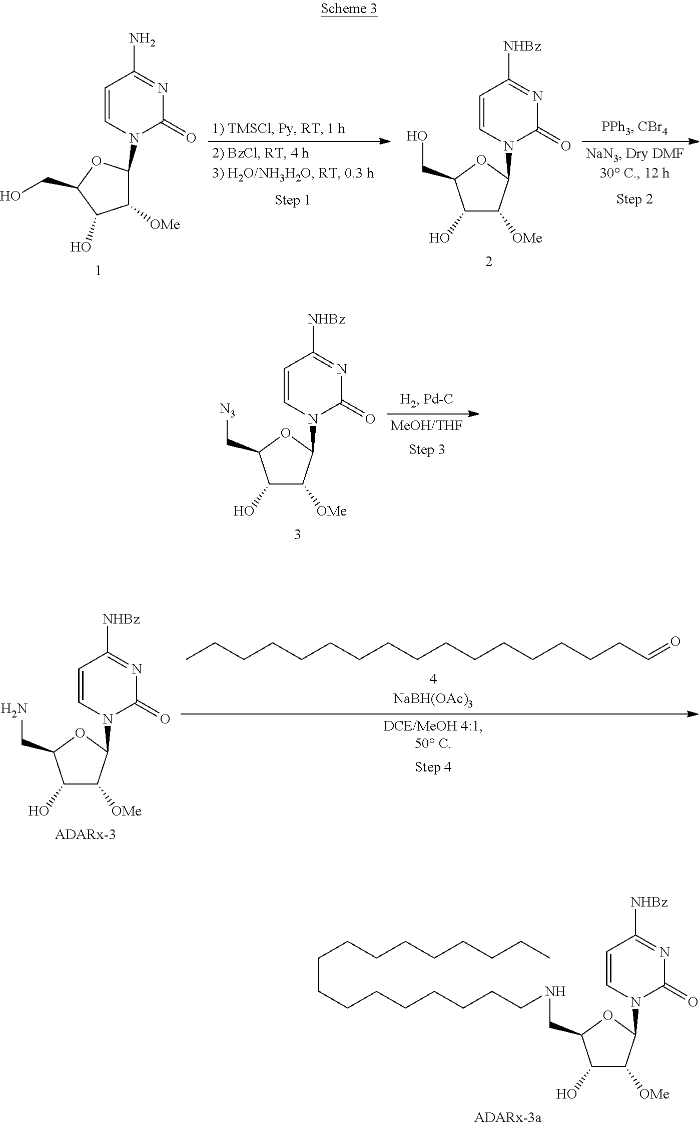

Step 1: To a mixture of 4-amino-1-[(2R,3R,4R,5R)-4-hydroxy-5-(hydroxymethyl)-3-methoxy-tetrahydrofuran-2-yl]pyrimidin-2-one 1 (5 g, 19.44 mmol, 1. q) in Py, (90 mL) was added TMSCl (7.60 g, 69.97 mmol, 8.88 mL, 3.6 eq) at 25° C. After stirring for 1 hour, the reaction mixture was cooled to 0° C., and BzCl (3.28 g, 23.32 mmol, 2.71 mL, 1.2 eq) was added dropwise by syringe. The ice-bath was then removed and the reaction mixture stirred at 25° C. for 4 hours. The reaction was quenched by the addition of H₂O (22 mL), after stirring for 5 min at 25° C., concentrated NH₃·H₂O (45 mL) was added and the mixture was stirred at 25° C. for 15 min. LC-MS showed the starting material was consumed completely and one main peak with desired m/z was detected. The resulting mixture was then evaporated to dryness under reduced pressure. The residue was stirred with water (110 mL) and filtered. After the solid product was washed with cold water (2×30 mL) and EtOH (2×30 mL), dried to give the title compound 2 (5 g, 13.84 mmol, 71.19% yield) as a white solid which was used into the next step without further purification. MS ES⁺: 361.9, ¹H NMR (400 MHz, DMSO-d₆) δ=11.70-10.79 (m, 1H), 8.55 (br d, J=7.4 Hz, 1H), 8.06-7.91 (m, 2H), 7.66-7.59 (m, 1H), 7.55-7.46 (m, 2H), 7.34 (br d, J=6.1 Hz, 1H), 5.89 (d, J=2.5 Hz, 1H), 5.25 (t, J=5.0 Hz, 1H), 5.14 (d, J=6.8 Hz, 1H), 4.15-4.03 (m, 1H), 3.96-3.85 (m, 1H), 3.84-3.70 (m, 2H), 3.62 (ddd, J=3.1, 4.8, 12.4 Hz, 1H), 3.48 (s, 3H).

Step 2: To a solution of N-[l-[(2R,3R,4R,5R)-4-hydroxy-5-(hydroxymethyl)-3-methoxy-tetrahydrofuran-2-yl]-2-oxo-pyrimidin-4-yl]benzamide 2 (5 g, 13.84 mmol, 1 eq) in DMF (100 mL) was added PPh₃ (7.26 g, 27.67 mmol, 2 eq), NaN₃ (5.40 g, 83.02 mmol, 6 eq) and CBr₄ (9.18 g, 27.67 mmol, 2 eq) sequently. The mixture was stirred at 30° C. for 12 hours. LC-MS showed the starting material was consumed completely and one main peak with desired m/z was detected. The reaction mixture was diluted with EtOAc (200 mL) and filtered to give a filtrate, then concentrated under reduced pressure to give a residue. The residue was purified by flash silica gel chromatography (ISCO®; 120 g SepaFlash® Silica Flash Column, Eluent of Ethyl acetate/Petroleum ethergradient 0-50% 30 min, 50-50% 180 min, 50%-100% 20 min, 100%-100% 90 min@100 mL/min) to give the title compound 3 (4.28 g, 11.08 mmol, 80.06% yield) as a white solid. MS ES⁺: 387.0, ¹H NMR (400 MHz, DMSO-d₆) δ=11.32 (s, 1H), 8.20 (br d, J=7.4 Hz, 1H), 8.05-7.95 (m, 2H), 7.67-7.60 (m, 1H), 7.55-7.48 (m, 2H), 7.42 (br d, J=7.4 Hz, 1H), 5.91 (d, J=2.9 Hz, 1H), 5.37 (br s, 1H), 4.08-3.96 (m, 2H), 3.87 (br dd, J=3.0, 4.1 Hz, 1H), 3.80-3.63 (m, 2H), 3.46 (s, 3H).

Step 3: A mixture of N-[1-[(2R,3R,4R,5R)-5-(azidomethyl)-4-hydroxy-3-methoxy-tetrahydrofuran-2-yl]-2-oxo-pyrimidin-4-yl]benzamide 3 (4.28 g, 11.08 mmol, 1 eq), Pd/C (0.8 g, 10% purity) in MeOH (40 mL) and THF (100 mL) was degassed and purged with H₂ for 3 times, and then the mixture was stirred at 25° C. for 2 hours under H₂ (15 Psi). LC-MS showed the starting material was consumed completely and one main peak with desired m/z was detected. The combined organic layers were filtered and concentrated under reduced pressure to give a residue. The residue was purified by flash silica gel chromatography (ISCO®; 80 g SepaFlash® Silica Flash Column, Eluent of 0-10% MeOH/DCM @80 mL/min) to give the title compound ADARx-3 (3.1 g, 8.60 mmol, 77.66% yield) as a white solid. MS ES⁺: 361.0, ¹H NMR (400 MHz, DMSO-d₆) δ=8.59 (d, J=7.5 Hz, 1H), 8.07-7.92 (m, 2H), 7.68-7.58 (m, 1H), 7.55-7.47 (m, 2H), 7.35 (d, J=7.5 Hz, 1H), 5.88 (d, J=2.9 Hz, 1H), 5.18-5.00 (m, 1H), 4.15-3.95 (m, 1H), 3.91-3.69 (m, 2H), 3.49-3.41 (m, 3H), 2.96-2.77 (m, 2H).

Step 4: To a solution of N-[1-[(2R,3R,4R,5R)-5-(aminomethyl)-4-hydroxy-3-methoxy-tetrahydrofuran-2-yl]-2-oxo-pyrimidin-4-yl]benzamide ADARx-3 (2.5 g, 6.94 mmol, 1 eq) and heptadecanal (1.77 g, 6.94 mmol, 1 eq) in DCE:MeOH=4:1 (40 mL) was added 4A MS (800 mg, 6.94 mmol, 1 eq) and TEA (1.40 g, 13.87 mmol, 1.93 mL, 2 eq), after stirring at 50° C. for 1 hour, NaBH(OAc)₃ (5.88 g, 27.75 mmol, 4 eq) was added. The mixture was stirred at 50° C. for 2 hour. LC-MS and TLC (DCM:MeOH=10:1, Rf=0.35) showed the start material was consumed completely and one main peak with desired m/z was detected. The reaction mixture was filtered and concentrated under reduced pressure to remove most of solvent. The residue was diluted with sat. NaHCO₃ (aq.) (500 mL) and extracted with CHCl₃/MeOH (10/1) (300 mL×3). The combined organic layers were dried over Na₂SO₄, filtered and concentrated under reduced pressure to give a residue. The residue was purified by flash silica gel chromatography (ISCO®; 80 g SepaFlash® Silica Flash Column, Eluent of 0-5% MeOH/DCM @80 mL/min) to give the title compound ADARx-3a (2 g, 3.34 mmol, 48.14% yield) as a yellow solid. MS ES⁺: 599.5, ¹H NMR (400 MHz, DMSO-d₆) δ=8.63 (d, J=7.6 Hz, 1H), 8.01 (d, J=7.4 Hz, 2H), 7.66-7.58 (m, 1H), 7.55-7.44 (m, 2H), 7.37 (d, J=7.5 Hz, 1H), 5.86 (d, J=2.3 Hz, 1H), 5.35-4.87 (m, 1H), 4.04-3.96 (m, 1H), 3.95-3.86 (m, 1H), 3.75 (dd, J=2.4, 4.7 Hz, 1H), 3.47 (s, 3H), 2.94-2.76 (m, 2H), 2.65-2.53 (m, 2H), 1.42 (br d, J=6.8 Hz, 2H), 1.27-1.13 (m, 28H), 0.84 (br t, J=6.8 Hz, 3H).

### Synthesis of ADARx-4 and ADARx-4a

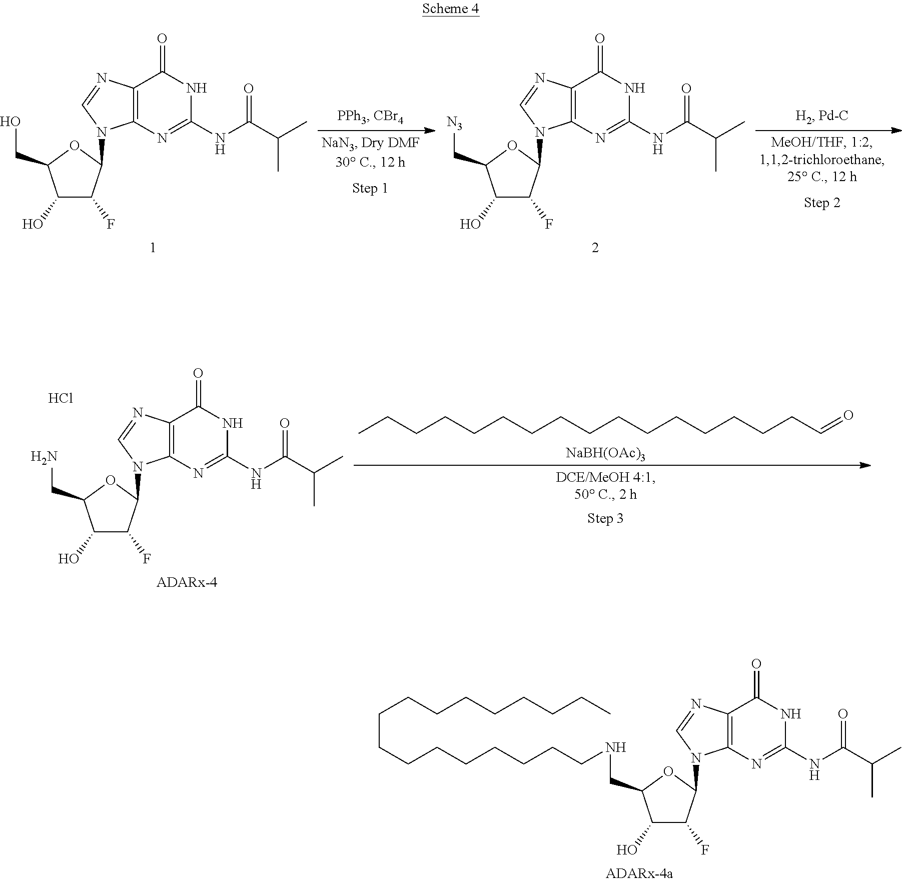

Step 1: N-(9-((2R,3R,4R,5R)-5-(azidomethyl)-3-fluoro-4-hydroxytetrahydrofuran-2-yl)-6-oxo-6,9-dihydro-1H-purin-2-yl)isobutyramide 2. To a solution of N-[9-[(2R,3R,4R,5R)-3-fluoro-4-hydroxy-5-(hydroxymethyl)tetrahydrofuran-2-yl]-6-oxo-1H-purin-2-yl]-2-methyl-propanamide 1 (15 g, 42.22 mmol, 1 eq) in DMF (300 mL) was added PPh₃ (22.14 g, 84.43 mmol, 2 eq) and NaN₃ (16.47 g, 253.29 mmol, 6 eq). Then CBr₄ (28.00 g, 84.43 mmol, 2 eq) was added in portions at 0° C. The mixture was stirred at 30° C. for 12 hours under N₂. The reaction mixture was diluted with ethyl acetate (700 mL) and filtered to give a filtrate, then concentrated under reduced pressure to give a residue. The residue was purified by flash silica gel chromatography (ISCO®; 40 g SepaFlash® Silica Flash Column, Eluent of 0-10% MeOH/DCM gradient @100 mL/min) to afford N-[9-[(2R,3R,4R,5R)-5-(azidomethyl)-3-fluoro-4-hydroxy-tetrahydrofuran-2-yl]-6-oxo-1H-purin-2-yl]-2-methyl-propanamide 2 (16 g, 42.07 mmol, 99.65% yield) as a white solid. MS ES⁺: 381.0, ¹H NMR (400 MHz, DMSO-d₆) δ 12.13 (br s, 1H), 11.65 (br s, 1H), 8.19 (s, 1H), 6.17 (dd, J=2.3, 18.6 Hz, 1H), 5.88 (d, J=6.4 Hz, 1H), 5.57-5.34 (m, 1H), 4.58-4.39 (m, 1H), 4.10-4.05 (m, 1H), 3.76-3.67 (m, 1H), 3.67-3.58 (m, 1H), 2.85-2.72 (m, 1H), 1.13 (d, J=6.9 Hz, 6H).

Step 2: N-(9-((2R,3R,4R,5R)-5-(aminomethyl)-3-fluoro-4-hydroxytetrahydrofuran-2-yl)-6-oxo-6,9-dihydro-1H-purin-2-yl)isobutyramide hydrochloride ADARx-4. To a solution of N-[9-[(2R,3R,4R,5R)-5-(azidomethyl)-3-fluoro-4-hydroxy-tetrahydrofuran-2-yl]-6-oxo-1H-purin-2-yl]-2-methyl-propanamide 2 (15.3 g, 40.23 mmol, 1 eq) in MeOH/THF=1:2 (500 mL) was added Pd/C (5 g, 4.02 mmol, 10% purity, 0.1 eq) and 1,1,2-trichloroethane (8.05 g, 60.34 mmol, 5.59 mL, 1.5 eq) under N₂. The suspension was degassed under vacuum and purged with H₂ several times. The mixture was stirred under H₂ (15 psi) at 25° C. for 12 hours. The reaction mixture was diluted with DCM (2 L) and MeOH (1 L), then filtered and concentrated under reduced pressure. The crude product was triturated with EtOAc/MeOH=3:1 at 25° C. for 60 min to afford N-[9-[(2R,3R,4R,5R)-5-(aminomethyl)-3-fluoro-4-hydroxy-tetrahydrofuran-2-yl]-6-oxo-1H-purin-2-yl]-2-methyl-propanamide hydrochloride ADARx-4 (11.7 g, 29.94 mmol, 74.42% yield) as a white solid, MS ES⁺: 355.0, ¹H NMR (400 MHz, DMSO-d₆) δ 8.23 (s, 1H), 6.37-6.13 (m, 1H), 5.92 (br d, J=4.8 Hz, 1H), 5.55-5.30 (m, 1H), 4.93-4.72 (m, 1H), 4.14 (dt, J=2.9, 7.6 Hz, 1H), 3.27 (br dd, J=3.1, 13.6 Hz, 1H), 3.22-3.17 (m, 1H), 2.88 (td, J=6.8, 13.6 Hz, 1H), 1.14 (dd, J=1.5, 6.8 Hz, 6H).

Step 3 N-(9-((2R,3R,4R,5R)-3-fluoro-5-((heptadecylamino)methyl)-4-hydroxytetrahydrofuran-2-yl)-6-oxo-6,9-dihydro-1H-purin-2-yl)isobutyramide ADARx-4a. To a solution of N-[9-[(2R,3R,4R,5R)-5-(aminomethyl)-3-fluoro-4-hydroxy-tetrahydrofuran-2-yl]-6-oxo-1H-purin-2-yl]-2-methyl-propanamide hydrochloride ADARx-4 (11 g, 28.15 mmol, 1 eq) and heptadecanal (7.16 g, 28.15 mmol, 1 eq) in MeOH:DCE=1:4 (160 mL) was added 4A MS (3 g, 28.15 mmol, 1 eq) and TEA (7.12 g, 70.37 mmol, 9.79 mL, 2.5 eq). The mixture was stirred at 50° C. for 0.5 hour. Then NaBH(OAc)₃ (17.90 g, 84.44 mmol, 3 eq) was added in portions, then The mixture was stirred at 50° C. for 2 hours. The reaction mixture was filtered and concentrated under reduced pressure. The residue was diluted with sat. NaHCO₃ aq. 1000 mL and extracted with DCM 700 mL (100 mL×7). The combined organic layers were concentrated under reduced pressure to give a residue. The residue was purified by flash silica gel chromatography (ISCO®; 120 g SepaFlash® Silica Flash Column, Eluent of 0-4% MeOH/DCM gradient @100 mL/min) to afford N-[9-[(2R,3R,4R,5R)-3-fluoro-5-[(heptadecylamino)methyl]-4-hydroxy-tetrahydrofuran-2-yl]-6-oxo-1H-purin-2-yl]-2-methyl-propanamide ADARx-4a (6 g, 9.69 mmol, 34.43% yield, 95.71% purity) as a white solid, MS ES⁺: 593.5, ¹H NMR (400 MHz, METHANOL-d₄) δ 8.09 (s, 1H), 6.28-6.14 (m, 1H), 5.49-5.25 (m, 1H), 4.83-4.74 (m, 1H), 4.25-4.07 (m, 1H), 3.15-2.95 (m, 2H), 2.81-2.61 (m, 3H), 1.57-1.47 (m, 2H), 1.33-1.23 (m, 34H), 0.97-0.85 (m, 3H).

### Synthesis of ADARx-5 and ADARx-5a

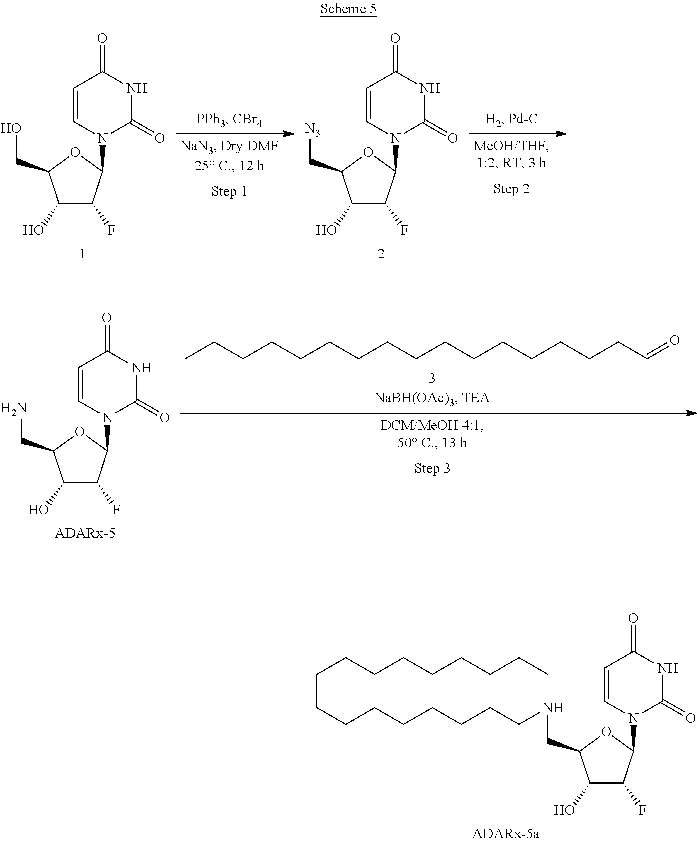 [SMILES: O=c1ccn([C@@H]2O[C@H](CO)[C@@H](O)[C@H]2F)c(=O)[nH]1 ; [N-]=[N+]=NC[C@H]1O[C@@H](n2ccc(=O)[nH]c2=O)[C@H](F)[C@@H]1O ; NC[C@H]1O[C@@H](n2ccc(=O)[nH]c2=O)[C@H](F)[C@@H]1O ; CCCCCCCCCCCCCCCCC=O ; CCCCCCCCCCCCCCCCCNC[C@H]1O[C@@H](n2ccc(=O)[nH]c2=O)[C@H](F)[C@@H]1O]

Step 1: To a solution of 1-[(2R,3R,4R,5R)-3-fluoro-4-hydroxy-5-(hydroxymethyl)tetrahydrofuran-2-yl]pyrimidine-2,4-dione 1 (15 g, 60.93 mmol, 1 eq) in DMF (300 mL) was added PPh₃ (31.96 g, 121.86 mmol, 2 eq), NaN₃ (23.77 g, 365.57 mmol, 6 eq) and CBr₄ (40.41 g, 121.86 mmol, 2 eq) sequently. The mixture was stirred at 25° C. for 12 hours. LC-MS and TLC (DCM:MeOH=20:1, R_{f}=0.25) showed the starting material was consumed completely and one main peak with desired m/z was detected. The reaction mixture was diluted with EA (600 mL) and filtered to give a filtrate, then concentrated under reduced pressure to give a residue. The residue was purified by flash silica gel chromatography (ISCO®; 330 g SepaFlash® Silica Flash Column, Eluent of 0-50-100% Ethyl acetate/Petroleum ethergradient @100 mL/min) to give the title compound 2 (16 g, crude) as a pale-yellow oil. MS ES⁺: 271.9, ¹H NMR (400 MHz, DMSO-d₆) δ=11.46 (d, J=1.3 Hz, 1H), 7.68 (d, J=8.1 Hz, 1H), 5.93-5.84 (m, 1H), 5.83-5.69 (m, 1H), 5.67 (dd, J=2.2, 8.1 Hz, 1H), 5.31-5.11 (m, 1H), 4.32-4.18 (m, 1H), 3.95 (ddd, J=2.8, 5.5, 8.1 Hz, 1H), 3.74 (dd, J=2.8, 13.7 Hz, 1H), 3.53 (dd, J=5.6, 13.7 Hz, 1H).

Step 2: To a solution of 1-[(2R,3R,4R,5R)-5-(azidomethyl)-3-fluoro-4-hydroxy-tetrahydrofuran-2-yl]pyrimidine-2,4-dione 2 (8 g, 29.50 mmol, 1 eq) in MeOH/THF=1/2 (250 mL) was added Pd/C (10% purity, 2 g) under N₂ atmosphere. The suspension was degassed and purged with H₂ for 3 times. The mixture was stirred under H₂ (15 Psi) at 25° C. for 3 hour. LC-MS showed the starting material was consumed completely and one main peak with desired m/z was detected. Combined two batches (8 g×2). The reaction mixture was filtered and concentrated under reduced pressure to give a residue. Then the residue was triturated by MeOH/EA=1/2 to give 1-((2R,3R,4R,5R)-5-(aminomethyl)-3-fluoro-4-hydroxytetrahydrofuran-2-yl)pyrimidine-2,4(1H,3H)-dione ADARx-5 (11 g, 44.86 mmol, 76.04% yield) as a white solid which was used into the next step without further purification. MS ES⁺: 246.0, ¹H NMR (400 MHz, DMSO-d₆) δ=7.95 (d, J=8.0 Hz, 1H), 5.87 (dd, J=1.9, 19.1 Hz, 1H), 5.61 (d, J=8.0 Hz, 1H), 5.15-4.99 (m, 1H), 4.18-4.07 (m, 1H), 3.77 (td, J=3.8, 7.5 Hz, 1H), 2.95-2.72 (m, 2H).

Step 3: To a solution of 1-[(2R,3R,4R,5R)-5-(aminomethyl)-3-fluoro-4-hydroxy-tetrahydrofuran-2-yl]pyrimidine-2,4-dione ADARx-5 (5 g, 20.39 mmol, 1 eq) in DCE/MeOH=4/1 (100 mL) was added heptadecanal (5.19 g, 20.39 mmol, 1 eq), 4A MS (1 g) and TEA (4.13 g, 40.78 mmol, 5.68 mL, 2 eq), after stirring at 50° C. for 1 hour, NaBH(OAc)₃ (17.29 g, 81.56 mmol, 4 eq) was added. The mixture was stirred at 50° C. for 12 hours. LC-MS and TLC (DCM:MeOH=10:1, R_{f}=0.20) showed the starting material was consumed completely and one main peak with desired m/z was detected. The reaction mixture was filtered and concentrated under reduced pressure to remove most of solvent. The residue was diluted with sat. NaHCO₃ (aq.) (1000 mL) and extracted with CHCl₃/MeOH (10/1) (300 mL×3). The combined organic layers were concentrated under reduced pressure to give a residue. The residue was purified by flash silica gel chromatography (ISCO®; 120 g SepaFlash® Silica Flash Column, Eluent of 0-5-12% MeOH/DCMgradient @60 mL/min) to give the title compound ADARx-5a (4.7 g, 9.72 mmol, 47.66% yield) as a white solid. MS ES⁺: 484.3 ¹H NMR (400 MHz, DMSO-d₆) δ=7.90 (d, J=8.0 Hz, 1H), 5.84 (dd, J=1.4, 19.5 Hz, 1H), 5.58 (d, J=8.1 Hz, 1H), 5.19-4.95 (m, 1H), 4.15-4.02 (m, 1H), 3.86 (br s, 1H), 2.91-2.72 (m, 2H), 2.56-2.52 (m, 2H), 1.38 (br d, J=6.3 Hz, 2H), 1.23 (s, 28H), 0.85 (br t, J=6.8 Hz, 3H).

### Synthesis of ADARx-6 and ADARx-6a

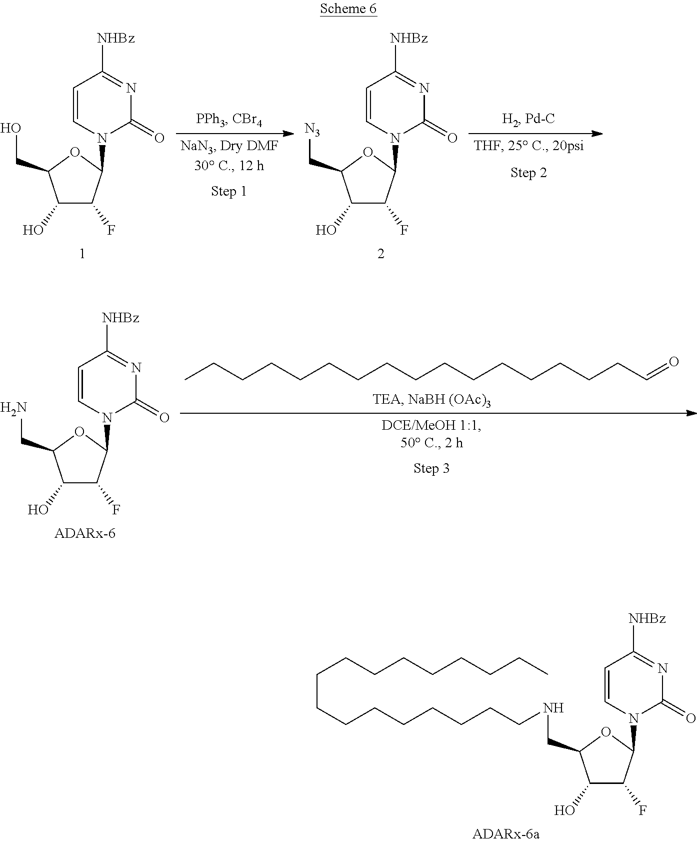

Step 1: To a solution of N-[1-[(2R,3R,4R,5R)-3-fluoro-4-hydroxy-5-(hydroxymethyl)tetrahydrofuran-2-yl]-2-oxo-pyrimidin-4-yl]benzamide 1 (15 g, 42.94 mmol, 1 eq) in DMF (100 mL) was added PPh₃ (22.53 g, 85.88 mmol, 2 eq), NaN₃ (16.75 g, 257.65 mmol, 6 eq) and CBr₄ (28.48 g, 85.88 mmol, 2 eq) at 0° C. The mixture was stirred at 30° C. for 12 hours. The reaction mixture was diluted with ethyl acetate (600 mL) and filtered to give a filtrate, then concentrated under reduced pressure to give a residue. The crude product was triturated with EtOH (300 mL) at 25° C. for 12 hours to afford the title compound 2 (15 g, 40.07 mmol, 93.32% yield) as a white solid. MS ES⁺:375.0

Step 2: To a solution of N-[1-[(2R,3R,4R,5R)-5-(azidomethyl)-3-fluoro-4-hydroxy-tetrahydrofuran-2-yl]-2-oxo-pyrimidin-4-yl]benzamide 2 (14 g, 33.66 mmol, 90% purity, 1 eq) in THF (400 mL) was added Pd/C (4.77 g, 10% purity) under N₂. The suspension was degassed under vacuum and purged with H₂ several times. The mixture was stirred under H₂ (20 psi) at 25° C. for 24 hours. The reaction mixture was filtered and concentrated under reduced pressure. TLC (DCM:MeOH=5:1) showed a new spot (R_{f}=0.42) was formed. The reaction mixture was filtered and washed with MeOH (4 L). The filter liquor was concentrated under reduced pressure. The residue was purified by flash silica gel chromatography (ISCO®; 40 g SepaFlash® Silica Flash Column, Eluent of 0-9% DCM/MeOH gradient @20 mL/min) to give the title compound ADARx-6 (8.2 g, 23.54 mmol, 69.94% yield) as a white solid. MS ES⁺:348.9, ¹H NMR (400 MHz, DMSO-d₆) δ 8.57 (d, J=7.6 Hz, 1H), 8.07-7.95 (m, 2H), 7.68-7.60 (m, 1H), 7.57-7.47 (m, 2H), 7.36 (d, J=7.5 Hz, 1H), 5.94 (d, J=18.4 Hz, 1H), 5.68-5.40 (m, 1H), 5.15-4.92 (m, 1H), 4.23-4.04 (m, 1H), 3.94-3.82 (m, 1H), 3.06-2.96 (m, 1H), 2.87 (dd, J=4.6, 14.0 Hz, 1H).

Step 3: To a solution of N-[1-[(2R,3R,4R,5R)-5-(aminomethyl)-3-fluoro-4-hydroxy-tetrahydrofuran-2-yl]-2-oxo-pyrimidin-4-yl]benzamide ADARx-6 (5 g, 14.35 mmol, 1 eq) and heptadecanal (3.65 g, 14.35 mmol, 1 eq) in DCE:MeOH=1:1 (100 mL) was added TEA (2.91 g, 28.71 mmol, 4.00 mL, 2 eq) and 4A MS (2 g, 14.35 mmol, 1 eq). After stirring for 1 hour, NaBH(OAc)₃ (12.17 g, 57.42 mmol, 4 eq) was added in portions. The mixture was stirred at 50° C. for 2 hours. TLC(DCM:MeOH=10:1) showed the desired compound (R_{f}=0.32) was formed. The reaction mixture was quenched by addition MeOH (50 mL) at 25° C., and then filtered and concentrated under reduced pressure to give a residue. The residue was diluted with sat. NaHCO₃ (aq.) (300 mL) and extracted with CHCl₃ (300 mL×3). The combined organic layers were concentrated under reduced pressure to give a residue. The residue was purified by flash silica gel chromatography (ISCO®; 20 g SepaFlash® Silica Flash Column, Eluent of 0-3% Ethyl acetate/Petroleum ether gradient @70 mL/min) to give the title compound ADARx-6 a (3 g, 5.11 mmol, 35.62% yield) as a light yellow solid. MS ES⁺: 587.3, ¹H NMR (400 MHz, DMSO-d₆) δ 8.59 (d, J=7.5 Hz, 1H), 8.05-7.96 (m, 2H), 7.68-7.58 (m, 1H), 7.56-7.46 (m, 2H), 7.37 (d, J=7.5 Hz, 1H), 5.93 (d, J=18.3 Hz, 1H), 5.56 (br d, J=4.3 Hz, 1H), 5.15-4.82 (m, 1H), 4.17-4.01 (m, 1H), 3.99-3.89 (m, 1H), 2.99-2.88 (m, 1H), 2.86-2.76 (m, 1H), 2.62-2.53 (m, 2H), 1.49-1.37 (m, 2H), 1.34-1.17 (m, 28H), 0.91-0.77 (m, 3H).

### Synthesis ADARx-7 and ADARx-7a

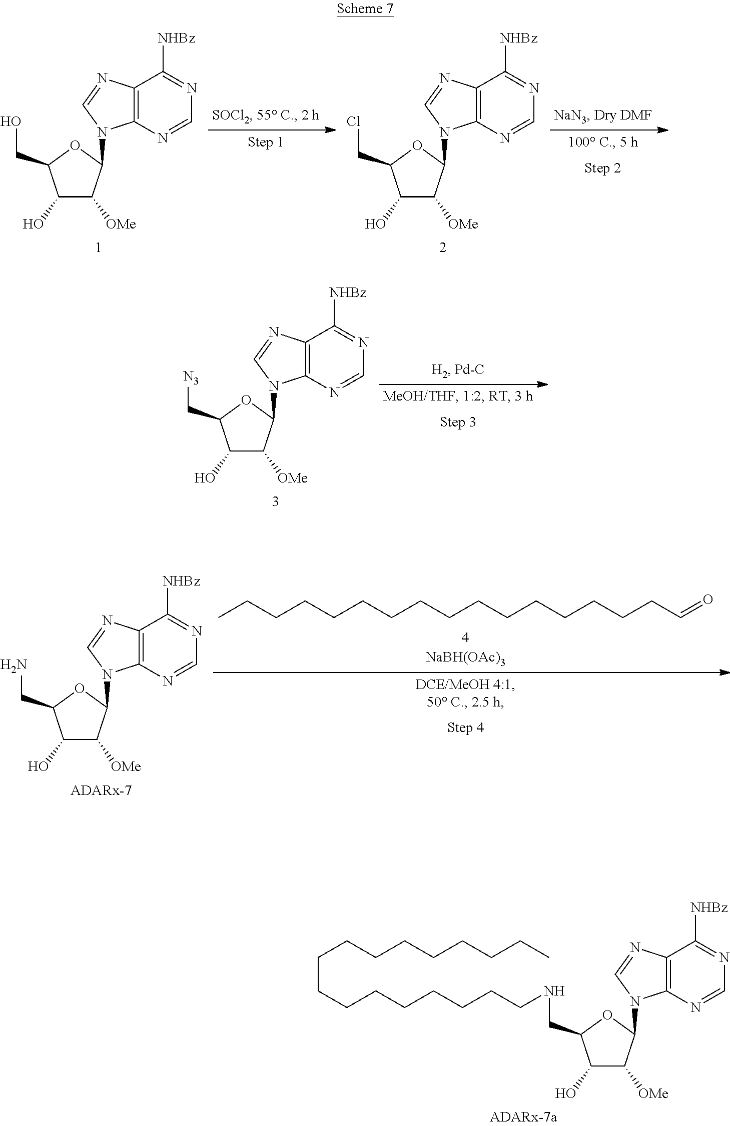

Step 1: N-[9-[(2R,3R,4R,5R)-4-hydroxy-5-(hydroxymethyl)-3-methoxy-tetrahydrofuran-2-yl]purin-6-yl]benzamide 1 (10 g, 25.95 mmol, 1 eq) was added portionwise to SOCl₂ (46.31 g, 389.23 mmol, 28.24 mL, 15 eq) over 5 min. Then the mixture was stirred at 55° C. for 2 hours. LC-MS showed the starting material was consumed completely and one main peak with desired m/z was detected. The reaction mixture was cooled to room temperature, then dropwise added sat. sodium bicarbonate aqueous (1000 mL) at 0° C. After warming to room temperature, the pH was confirmed as basic, then solids were collected by filtration. The crude product 2 (8.3 g, 20.55 mmol, 79.21% yield) was obtained as a white solid which was used into the next step without further purification. MS ES⁺: 404.0

Step 2: To a solution of N-[9-[(2R,3R,4S,5S)-5-(chloromethyl)-4-hydroxy-3-methoxy-tetrahydrofuran-2-yl]purin-6-yl]benzamide 2 (8.3 g, 20.55 mmol, 1 eq) in DMF (80 mL) was added NaN₃ (6.68 g, 102.77 mmol, 5 eq). The mixture was stirred at 100° C. for 5 hours. LC-MS showed the starting material was consumed completely and one main peak with desired m/z was detected. The reaction mixture was diluted with EtOAc (160 mL) and filtered to give a filtrate, then concentrated under reduced pressure to give a residue. The crude product 3 (8.8 g, crude) was obtained as a pale yellow solid which was used into the next step without further purification. MS ES⁺: 411.0

Step 3: To a solution of N-[9-[(2R,3R,4R,5R)-5-(azidomethyl)-4-hydroxy-3-methoxy-tetrahydrofuran-2-yl]purin-6-yl]benzamide 3 (8.8 g, 21.44 mmol, 1 eq) in MeOH/THF=1/2 (300 mL) was added Pd/C (10% purity, 1.8 g) under N₂ atmosphere. The suspension was degassed and purged with H₂ for 3 times. The mixture was stirred under H₂ (15 Psi) at 25° C. for 3 hours. LC-MS and TLC (DCM:MeOH=5:1, R_{f}=0.10) showed the starting material was consumed completely and one main peak with desired m/z was detected. The reaction mixture was filtered and concentrated under reduced pressure to give a residue. The residue was purified by flash silica gel chromatography (ISCO®; 40 g SepaFlash® Silica Flash Column, Eluent of 0-20% MeOH/DCM gradient @100 mL/min) to give the title compound ADARx-7 (6 g, 15.61 mmol, 72.79% yield) as an off white solid. MS ES⁺: 385.0, ¹H NMR (400 MHz, DMSO-d₆) δ=8.71 (d, J=8.9 Hz, 2H), 8.06 (br d, J=7.3 Hz, 2H), 7.65-7.57 (m, 1H), 7.57-7.49 (m, 2H), 6.10 (d, J=6.1 Hz, 1H), 4.52 (t, J=5.5 Hz, 1H), 4.42-4.33 (m, 1H), 3.96-3.86 (m, 1H), 3.34 (s, 3H), 2.91-2.73 (m, 2H).

To a solution of N-[9-[(2R,3R,4R,5R)-5-(aminomethyl)-4-hydroxy-3-methoxy-tetrahydrofuran-2-yl]purin-6-yl]benzamide ADARx-7 (6 g, 15.61 mmol, 1 eq) in DCE/MeOH=4/1 (300 mL) was added heptadecanal (3.97 g, 15.61 mmol, 1 eq), 4A MS (1 g) and TEA (3.16 g, 31.22 mmol, 4.35 mL, 2 eq), after stirring at 50° C. for 0.5 hour, NaBH(OAc)₃ (13.23 g, 62.44 mmol, 4 eq) was added in portions. Then the mixture was stirred at 50° C. for 2 hours. LC-MS and TLC (DCM:MeOH=10:1, R_{f}=0.20) showed the starting material was consumed completely and one main peak with desired m/z was detected. The reaction mixture was filtered and concentrated under reduced pressure to remove most of solvent. The residue was diluted with sat. NaHCO₃ (aq.) (1000 mL) and extracted with CHCl₃/MeOH (10/1) (300 mL×3). The combined organic layers were concentrated under reduced pressure to dryness which was purified by flash silica gel chromatography (ISCO®; 120 g SepaFlash® Silica Flash Column, Eluent of 0-5% MeOH/DCM gradient @100 mL/min) to give the title compound ADARx-7a (5 g, 8.03 mmol, 51.43% yield) as a white solid. MS ES⁺: 623.3, ¹H NMR (400 MHz, DMSO-d₆) δ=8.75 (d, J=2.4 Hz, 2H), 8.08-8.01 (m, 2H), 7.69-7.60 (m, 1H), 7.58-7.52 (m, 2H), 6.12 (d, J=6.0 Hz, 1H), 4.54 (t, J=5.5 Hz, 1H), 4.43-4.33 (m, 1H), 4.09-3.98 (m, 1H), 3.34 (s, 3H), 2.82 (dq, J=5.4, 12.6 Hz, 2H), 2.57-2.51 (m, 2H), 1.39 (d, J=6.5 Hz, 2H), 1.24-1.19 (m, 28H), 0.87-0.81 (m, 3H).

### Synthesis of NB-100: and ADARx-8a

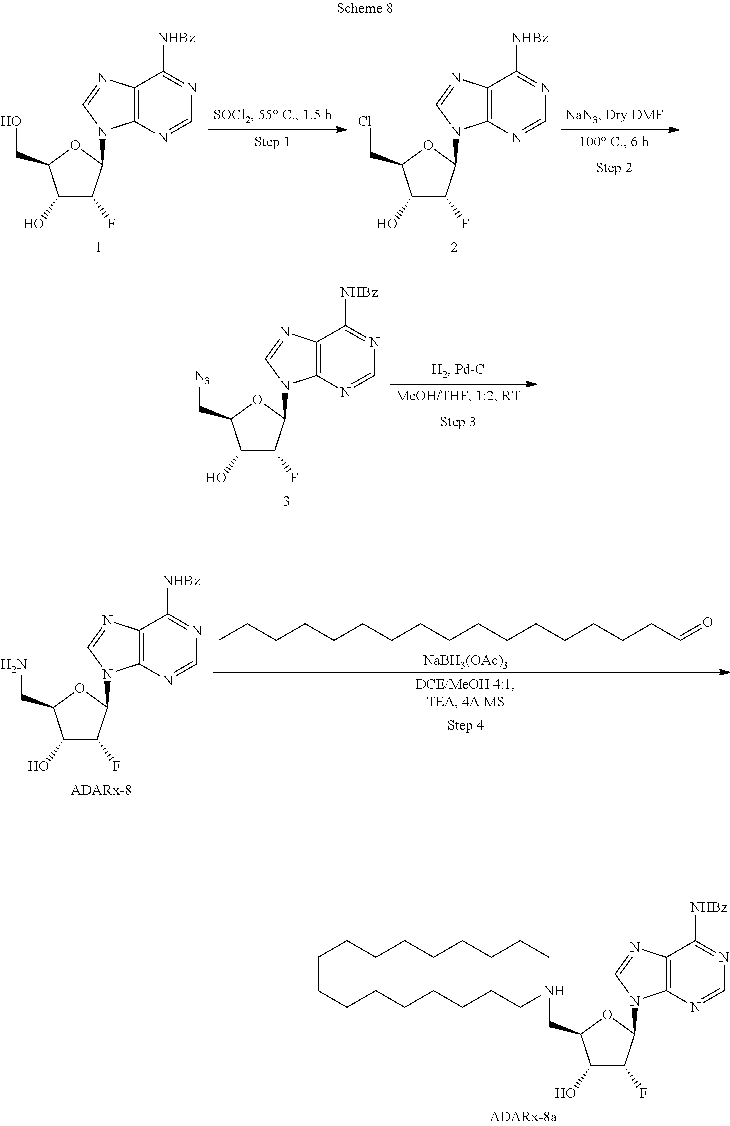

Step 1: To SOCl₂ (23.90 g, 200.89 mmol, 14.57 mL, 15 eq) was added N-[9-[(2R,3R,4R,5R)-3-fluoro-4-hydroxy-5-(hydroxymethyl)tetrahydrofuran-2-yl]purin-6-yl]benzamide 1 (5 g, 13.39 mmol, 1 eq) in portions. The mixture was stirred at 55° C. for 1.5 hours. The reaction mixture was cooled to room temperature, then added dropwise by pipette to stirring sat'd aqueous sodium bicarbonate (2500 mL) at 0° C. After warming to room temperature, the pH was confirmed as basic, then solids were collected by filtration. The filter cake was rinsed with water (200 mL), then dried under reduced pressure to give the title compound 2 (5.2 g, 13.27 mmol, 99.10% yield) as a white solid. MS ES⁺:391.9, ¹H NMR (400 MHz, DMSO-d₆) δ 11.25 (s, 1H), 8.78 (s, 1H), 8.63 (s, 1H), 8.09-7.98 (m, 2H), 7.69-7.61 (m, 1H), 7.60-7.50 (m, 2H), 6.43 (dd, J=1.9, 19.6 Hz, 1H), 6.01 (d, J=6.5 Hz, 1H), 5.79-5.56 (m, 1H), 4.80-4.63 (m, 1H), 4.20 (dt, J=3.3, 6.6 Hz, 1H), 4.07-3.98 (m, 1H), 3.94-3.85 (m, 1H).

Step 2: To a solution of N-[9-[(2R,3R,4R,5S)-5-(chloromethyl)-3-fluoro-4-hydroxy-tetrahydrofuran-2-yl]purin-6-yl]benzamide 2 (5.2 g, 13.27 mmol, 1 eq) in DMF (50 mL) was added NaN₃ (4.31 g, 66.36 mmol, 5 eq). The mixture was stirred at 100° C. for 6 hours. LCMS showed the desired compound was formed. The reaction mixture was diluted with ethyl acetate (300 mL) and filtered, the filter liquor were concentrated under reduced pressure to give the title compound 3 (5.2 g, 13.05 mmol, 98.35% yield) as a yellow solid, MS ES⁺: 399.0, ¹H NMR (400 MHz, DMSO-d₆) δ 11.25 (br s, 1H), 8.78 (s, 1H), 8.65 (s, 1H), 8.11-8.00 (m, 2H), 7.69-7.61 (m, 1H), 7.58-7.50 (m, 2H), 6.42 (dd, J=1.8, 19.9 Hz, 1H), 5.95 (br d, J=5.9 Hz, 1H), 5.76-5.53 (m, 1H), 4.86-4.64 (m, 1H), 4.17-4.03 (m, 1H), 3.81-3.69 (m, 1H), 3.58 (dd, J=5.7, 13.6 Hz, 1H).

Step 3: To a solution of N-[9-[(2R,3R,4R,5R)-5-(azidomethyl)-3-fluoro-4-hydroxy-tetrahydrofuran-2-yl]purin-6-yl]benzamide 3 (5.2 g, 13.05 mmol, 1 eq) in THF/MeOH=2:1 (300 mL) was added Pd/C (1.02 g, 3.92 mmol, 10% purity) under N₂. The suspension was degassed under vacuum and purged with H₂ several times. The mixture was stirred under H₂ (15 psi) at 25° C. for 1 hour. TLC (DCM:MeOH=5:1) showed a new spot (R_{f}=0.28) was formed. The reaction mixture was concentrated under reduced pressure. The residue was purified by flash silica gel chromatography (ISCO®; 40 g SepaFlash® Silica Flash Column, Eluent of 0-18% MeOH/DCM gradient @80 mL/min) to give the title compound ADARx-8 (2.5 g, 6.71 mmol, 51.43% yield) as a white solid. MS ES⁺: 373.0, ¹H NMR (400 MHz, DMSO-d₆) δ 8.76 (d, J=8.8 Hz, 2H), 8.09-8.00 (m, 2H), 7.68-7.62 (m, 1H), 7.59-7.52 (m, 2H), 6.37 (dd, J=2.7, 18.1 Hz, 1H), 5.90-5.70 (m, 1H), 5.69-5.50 (m, 1H), 4.68-4.49 (m, 1H), 4.04-3.90 (m, 1H), 3.03-2.95 (m, 1H), 2.86 (dd, J=5.8, 13.8 Hz, 1H).

Step 4: To a solution of N-[9-[(2R,3R,4R,5R)-5-(aminomethyl)-3-fluoro-4-hydroxy-tetrahydrofuran-2-yl]purin-6-yl]benzamide ADARx-8 (2 g, 5.37 mmol, 1 eq) and heptadecanal (1.37 g, 5.37 mmol, 1 eq) in MeOH:DCE=1:4 (40 mL) was added TEA (1.09 g, 10.74 mmol, 1.50 mL, 2 eq) and 4A MS (800 mg, 5.37 mmol, 1 eq), after stirring for 1 hr, NaBH(OAc)₃ (4.55 g, 21.48 mmol, 4 eq) was added. The mixture was stirred at 50° C. for 2 hours. TLC(DCM:MeOH=10:1, R_{f}=0.30) showed the desired compound was formed. The reaction mixture was concentrated under reduced pressure. The residue was diluted with sat. NaHCO₃ (aq.) (100 mL) and extracted with CHCl₃ (100 mL×3). The combined organic layers were concentrated under reduced pressure to give a residue. The residue was purified by flash silica gel chromatography (ISCO®; 20 g SepaFlash® Silica Flash Column, Eluent of 0-5% Ethyl acetate/Petroleum ether gradient @80 mL/min) to afford the title compound ADARx-8a (1.35 g, 2.21 mmol, 41.15% yield) as a white solid. MS ES+: 611.3, ¹H NMR (400 MHz, DMSO-d₆) δ=8.73 (d, J=17.6 Hz, 2H), 8.11-8.00 (m, 2H), 7.65 (s, 1H), 7.58-7.51 (m, 2H), 6.35 (dd, J=2.4, 18.3 Hz, 1H), 5.75 (br d, J=2.1 Hz, 1H), 5.68-5.39 (m, 1H), 4.68-4.48 (m, 1H), 4.10-3.96 (m, 1H), 2.95-2.84 (m, 1H), 2.78 (dd, J=5.9, 13.1 Hz, 1H), 2.52 (br s, 2H), 1.36 (br d, J=5.8 Hz, 2H), 1.26-1.15 (m, 28H), 0.88-0.80 (m, 3H).

### Example 8: NB-108

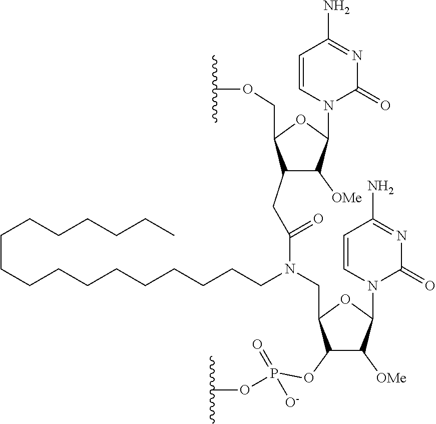

### Synthesis of NB-108

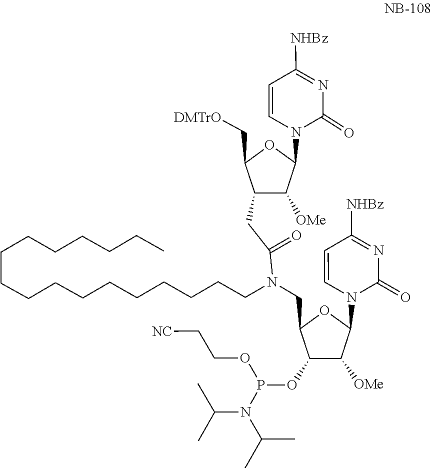

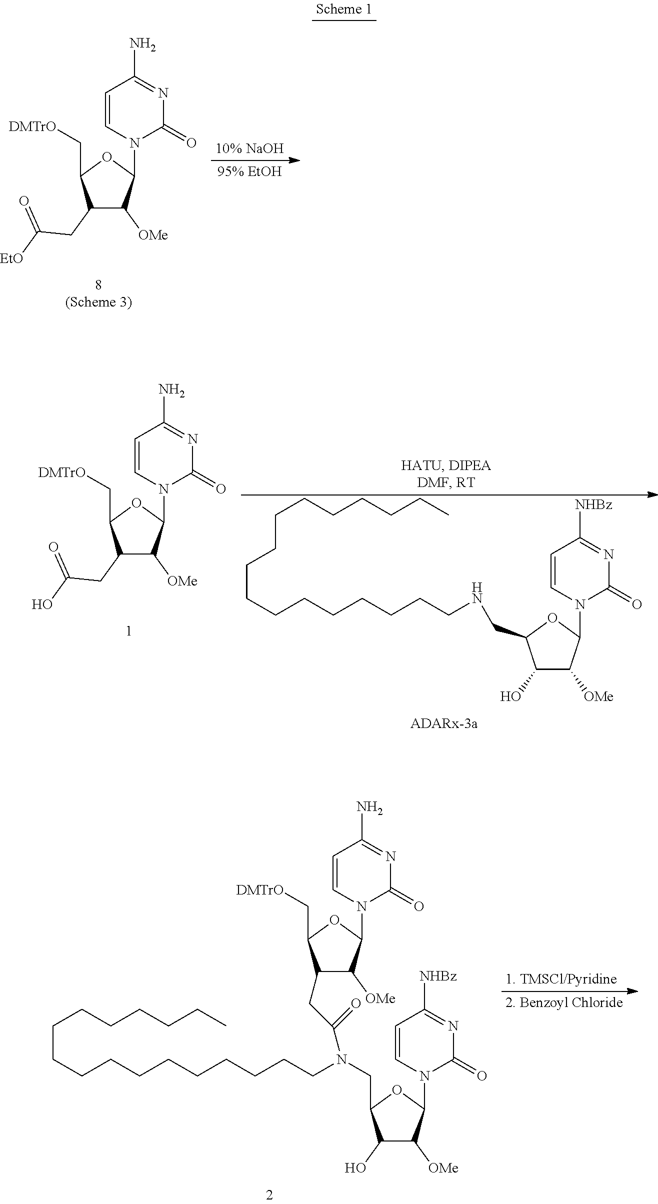

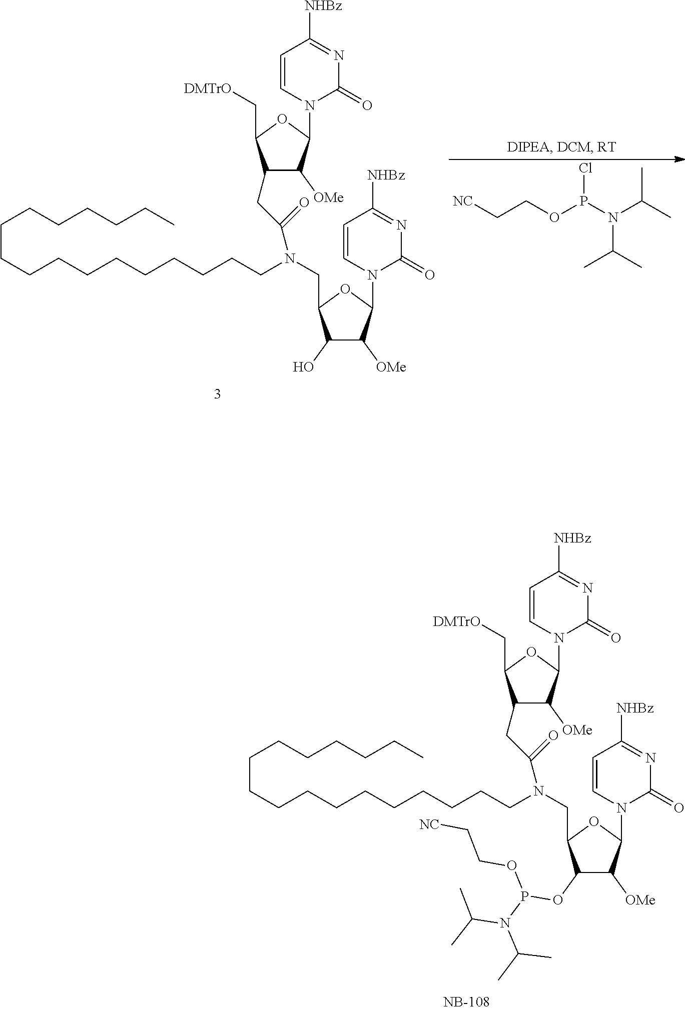

10% aqueous NaOH solution (0.6 mL) was added to a solution of ester (1.9 g, 3.021 mmol) in 95% ethanol (10 mL) and the resulting mixture was stirred for 1 h at 40° C. LCMS showed complete hydrolysis, Ethanol was evaporated the residue was diluted with water 5 mL, acidified by the careful addition of a 2N aqueous HCl solution until the pH 6.5. resulting solids were filtered and dried under high vacuum to obtain acid 1 (1.8 g, 99%) as a white solid. LCMS: m/z=601 (M+)

A solution of acid 1 (720 mg, 1.198 mmol, 1 eq), DIPEA (0.625 mL, 3.594 mmol, 3 eq) and HATU (683 mg, 1.797 mmol, 1.5 eq) in DMF (10 mL) was stirred for 15 min at room temperature, then amine (716 mg, 1.198 mmol, 1 eq) was added and the mixture was stirred for 12 h at room temperature, reaction mixture was diluted with water (20 ml), extracted with Ethyl acetate 2×100 mL, combined organics were washed with Aq. Sat. NaHCO3, and brine solution, dried over Na₂SO₄, and concentrated, the crude residue was purified by Ethyl acetate/Hex, 0-100% as an eluent, pure fractions were combined and concentrated to obtain amide (1.23 g, 85%) as a brown solid. LCMS: m/z 1205 (M+Na).

To a solution of amine 2 (1.23 g, 1.041 mmol, 1 eq) in anhydrous pyridine (10 mL) at RT was added chlorotrimethylsilane (0.264 mL, 2.081 mmol, 2 eq) dropwise. The mixture was stirred for 2 hours, then benzoyl chloride (0.242 mL, 2.081 mmol, 2 eq) was added dropwise to the reaction mixture. The reaction was stirred overnight at RT. H20 (2 mL) was added to the reaction, and the reaction was stirred for 3 hours. The solvent was removed by rotavapor. The crude mixture was partitioned between H2O and EtOAc. The aqueous phase was extracted with EtOAc (200 mL). The combined organic layers were washed with brine, dried with Na₂SO₄, filtered, and concentrated. The residue was purified by column chromatography using 0-100% EtOAc in Hexanes to give product 3 (600 mg, 45%) as a white solid. LCMS: 1308 (M+Na).

To a stirred solution of alcohol 3 (0.560 g, 0.435 mmol, 1 eq) and diisopropylethylamine (0.454 mL, 2.613 mmol, 6 eq), in DCM (10 mL), was added N, N-diisopropyl chlorophosphoramidite (0.291 mL 1.306 mmol, 3 eq) dropwise. The reaction mixture was stirred at room temperature for 1 h. Reaction mixture was quenched with Aq. Saturated NaHCO₃ solution (20 mL), extracted with DCM (2×50) ml, washed with brine (50 mL), dried over Na₂SO₄, and evaporated, the crude product was loaded on to (pre-equilibrated two times with 1% Et₃N-DCM) Biotage silica gel column (25 g 20 m), and purified by flash chromatography using 0-10% MeOH/DCM containing 1% Et₃N as an additive. Pure fractions were combined and concentrated, dried under high vacuum to obtain Phosphoramidite NB-108 (180 mg, 27%) as a beige solid. 92% purity by HPLC, Mass (m/z 1508 M+Na), ³¹P NMR (202 MHz, DMSO-d₆) δ 149.43, 149.16, 149.09, 148.81.

Oligonucleotide Comprising Dinucleotide NB-108 has been Synthesized Using the General Procedure Described in Example 1.

### Example 9: NB-113

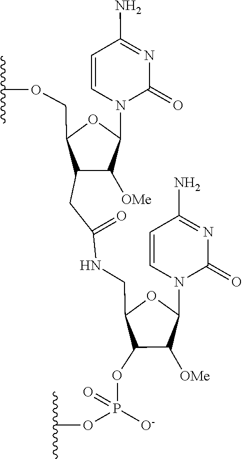

### Synthesis of Compound NB-113

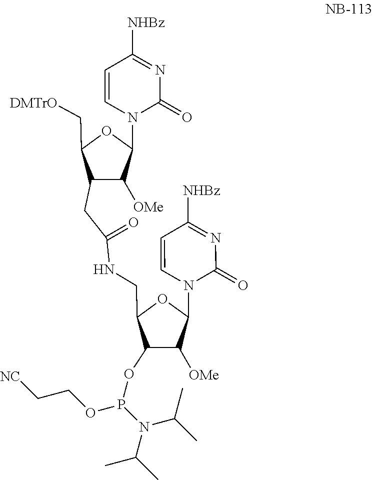

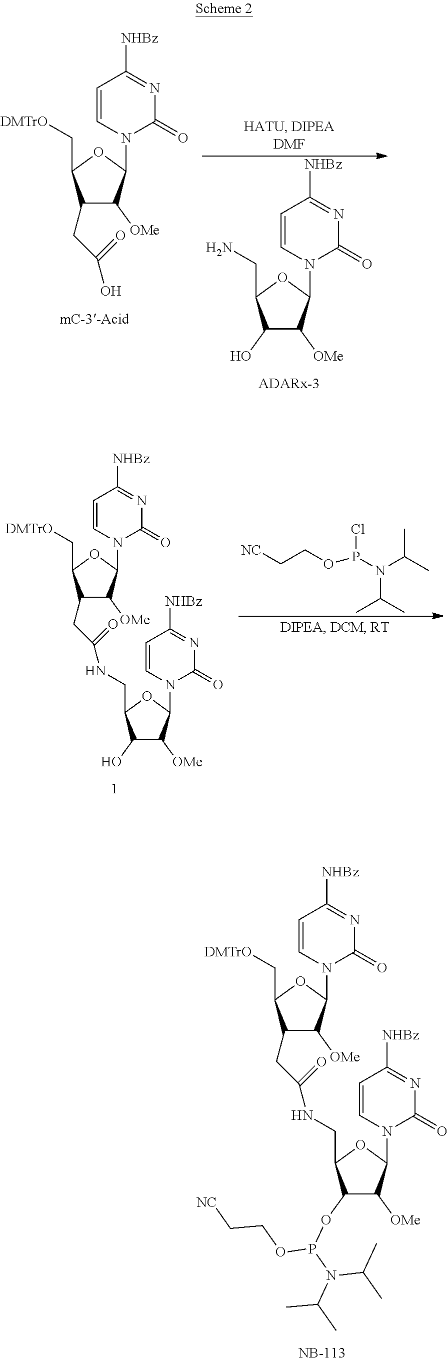

Add HATU (1.54 g, 4.04 mmol, 1.5 equiv) to a suspension of mC Acid (1.9 g, 2.7 mmol, 1.0 equiv), mC amine (1.06 g, 2.96 mmol, 1.1 equiv), and DIPEA (1.4 mL, 8.1 mmol, 3.0 equiv) in anhydrous DMF (25 mL) at room temperature. The white suspension turned yellow and dissolved within 30 seconds. After 30 min, full conversion observed with PR M+H=1048.9 observed, very clean, no ester observed. After 2.5 h, the reaction mixture was added dropwise to stirring 50% sat'd NaHCO₃ (250 mL). The resulting solids were collected by filtration in a 150 mL fritted funnel. The solids were washed with water (40 mL) and dried with open vacuum overnight. The resulting white solids (2.9 g) were added to stirring EtOAc (100 mL)—not dissolving. The mixture was diluted with methanol and DCM (˜100 mL), not all dissolves but >90%, dried with anhydrous sodium sulfate. The crude reaction mixture was filtered through Celite and concentrated under reduced pressure. The crude residue was purified by FCC on silica gel (80 g gold, 0470% EtOAc-EtOH (3:1)/Heptane; 0%[1], 0→70% [5, f-12], 70%[6,13-31]). Collected f18-26 to afford amide 1 (2.17 g, 77% yield) as a white solid. HPLC purity 97%. NMR clean and consistent.

Add CE-DIP-Cl (1.2 mL, 5.2 mmol, 2.5 equiv) to a solution of alcohol 1 (2.17 g, 2.07 mmol, 1 equiv) and DIPEA (1.8 mL, 10 mmol, 5.0 equiv) in DCM (12 mL) at 0° C., then stir at RT. After 2 h, full conversion observed PR M+H-DIPA+OH]+=1165.7 with ˜5% oxidation, the reaction mixture was diluted with 50% sat'd NaHCO₃ (20 mL) and extracted with DCM (100 mL). The organic layer was dried over anhydrous sodium sulfate, filtered, and concentrated under reduced pressure. The resulting residue was redissolved in DCM and concentrated. Then, redissolve in minimal DCM and triturate with hexanes to precipitate product. The supernatant was decanted. The residual solvent was concentrated, then the reaction mixture was dissolved in DCM and purified by FCC on silica gel (120 g, (1) 0-100% [3CV] EtOAc/Heptane+1% NEt3; (2) 0-2% MeOH/EtOAc+1% NEt₃; 0%[2, f1-10], 0-2%[5, f11-40]). TLC shows two spots by LCMS shows very clean single peak for fr12, 25, 30, and 38. Collected f12-38 to afford amidite, 96% HPLC purity. By MS lots of 219 mass for reagent. The collected fractions were concentrated under reduced pressure and triturated with DCM/hexanes as above (2×)—LCMS still shows lots of 219 peak, not very effective. The volatiles were removed and the amidite was suspended in diethyl ether (60 mL) and sonicated. Diluted with hexanes (20 mL) to remove some PR observed in supernatant, then decanted and concentrated under reduced pressure. Triturated with DCM/hexane to remove ether to afford amidite NB-113 (1.94 g, 77% yield; HPLC purity 96%, Mass, M+Na=1270.9 and M−H=1246.4, ³¹P NMR (202 MHz, DMSO-d₆) δ 149.90, 149.82.

Oligonucleotide Comprising Dinucleotide NB-113 has been Synthesized Using the General Procedure Described in Example 1.

### Example 10: NB-118

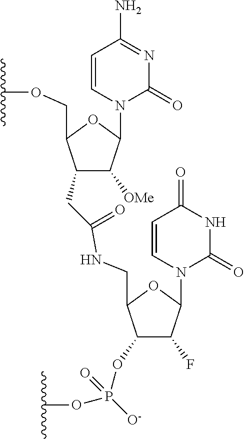 [SMILES: *OCC1OC(n2ccc(N)nc2=O)[C@H](OC)[C@@H]1CC(=O)NCC1OC(n2ccc(=O)[nH]c2=O)[C@H](F)[C@@H]1OP(=O)([O-])O*]

### Synthesis of Compound NB-118

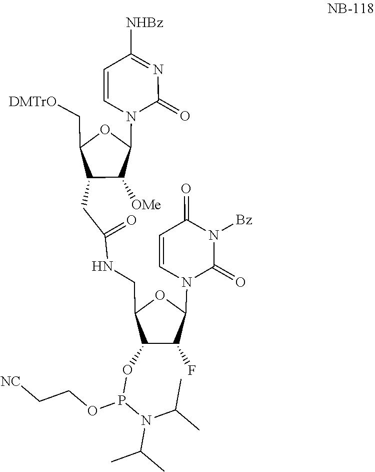

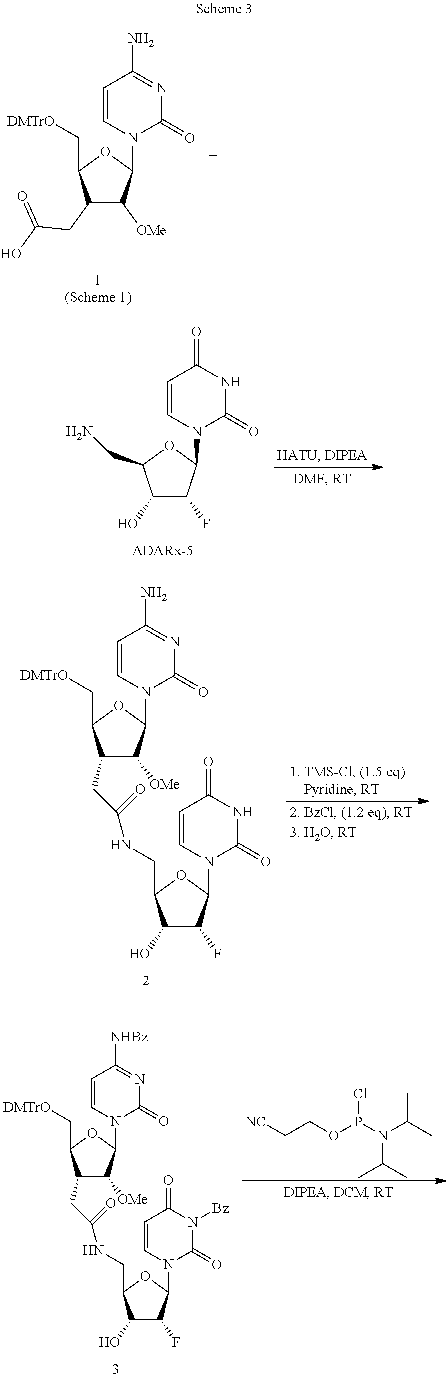

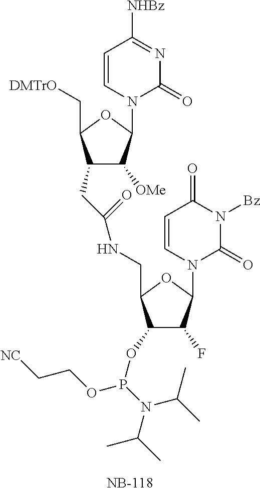

A solution of acid 1 (1 g, 1.664 mmol, 1 eq), DIPEA (0.868 mL, 4.992 mmol, 3 eq) and HATU (0.948 g, 2.496 mmol, 1.5 eq) in DMF (10 mL) was stirred for 15 min at room temperature, then amine ADARx-5 (0.428 g, 1.747 mmol, 1.05 eq) was added and the mixture was stirred for 3 h at room temperature, reaction mixture was diluted with water (50 ml), precipitated solids were filtered and washed with water, the solids were dried and re dissolved in DCM then purified by Ethyl acetate/MeOH, 0-5% as an eluent, pure fractions were combined and concentrated to obtain amide 2 (0.9 g, 65%) as a brown solid. NMR and LCMS m/z 851 (M+Na).

To a mixture of amine 2 (870 mg, 1.051 mmol, 1 eq) in pyridine (10 mL) was added TMSCl (0.2 mL, 1.576 mmol, 1.5 eq) after stirring for 2 h, at room temperature LCMS showed formation TMS protection, then benzoyl chloride (0.427 mL, 3.678 mmol, 3.5 eq) was added and the reaction mixture was stirred at RT for 12 h. LCMS showed Bz-protection. The reaction was quenched by the addition of water (1 mL), stirred for 12 h at room temperature. LCMS showed TMS deprotection. Reaction mixture was diluted with water 50 mL, extracted with DCM, 2×100 mL, washed with brine 50 mL, dried over Na₂SO₄, evaporated, the crude residue was purified by column chromatography using 0-100% Ethyl acetate/hexane as eluent pure fractions were combined and concentrated to obtain product 3 (300 mg, 27%) as a white solid. LCMS m/z=1035 (M−2).

To a stirred solution of alcohol 3 (0.260 g, 0.251 mmol, 1 eq) and diisopropylethylamine (0.262 mL, 1.504 mmol, 6 eq), in DCM (10 mL), was added N, N-diisopropyl chlorophosphoramidite (0.484 mL 2.177 mmol, 3 eq) dropwise. The reaction mixture was stirred at room temperature for 2 h. Reaction mixture was quenched with Aq. Saturated NaHCO₃ solution (30 mL), extracted with DCM (50) ml, washed with brine (50 mL), dried over Na₂SO₄, and evaporated the crude product was loaded on to (pre-equilibrated two times with 1% Et₃N-DCM) Biotage silica gel column (25 g 20 m), and purified by flash chromatography using 0-10% MeOH/DCM containing 1% Et₃N as an additive. Pure fractions were combined and concentrated, dried under high vacuum to obtain Phosphoramidite NB-118 (220 mg, 69%) product as a white solid. 96% purity by HPLC, Mass (m/z 1259 M+Na), ³¹P-NMR (202, MHz, DMSO-d6) δ 150.72, 150.69, 150.47, 150.41.

A solution of acid ADAR-15 (0.900 g, 1.525 mmol, 1 eq), DIPEA (0.796 mL, 4.576 mmol, 3 eq) and HATU (0.869 g, 2.288 mmol, 1.5 eq) in DMF (10 mL) was stirred for 15 min at room temperature, then amine ADARx-1 (0.614 g, 1.678 mmol, 1.1 eq) was added and the mixture was stirred for 3 h at room temperature, reaction mixture was diluted with water (50 ml), precipitated solids were filtered and washed with water, the solids were dried and re dissolved in DCM then purified by Ethyl acetate/MeOH, 0-5% as an eluent, pure fractions were combined and concentrated to obtain amide 1 (0.89 g, 56%) as a brown solid. LCMS m/z 961 (M+Na).

Oligonucleotide Comprising Dinucleotide NB-118 has been Synthesized Using the General Procedure Described in Example 1.

### Example 11: NB-120

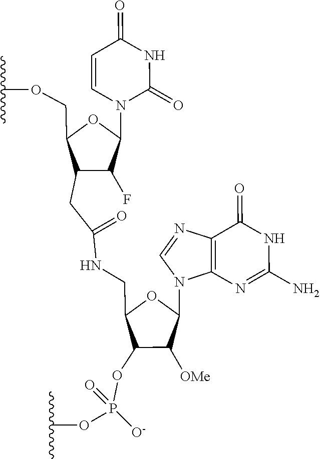

### Synthesis of NB-120

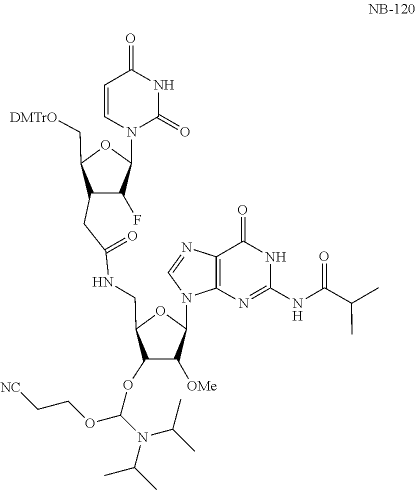

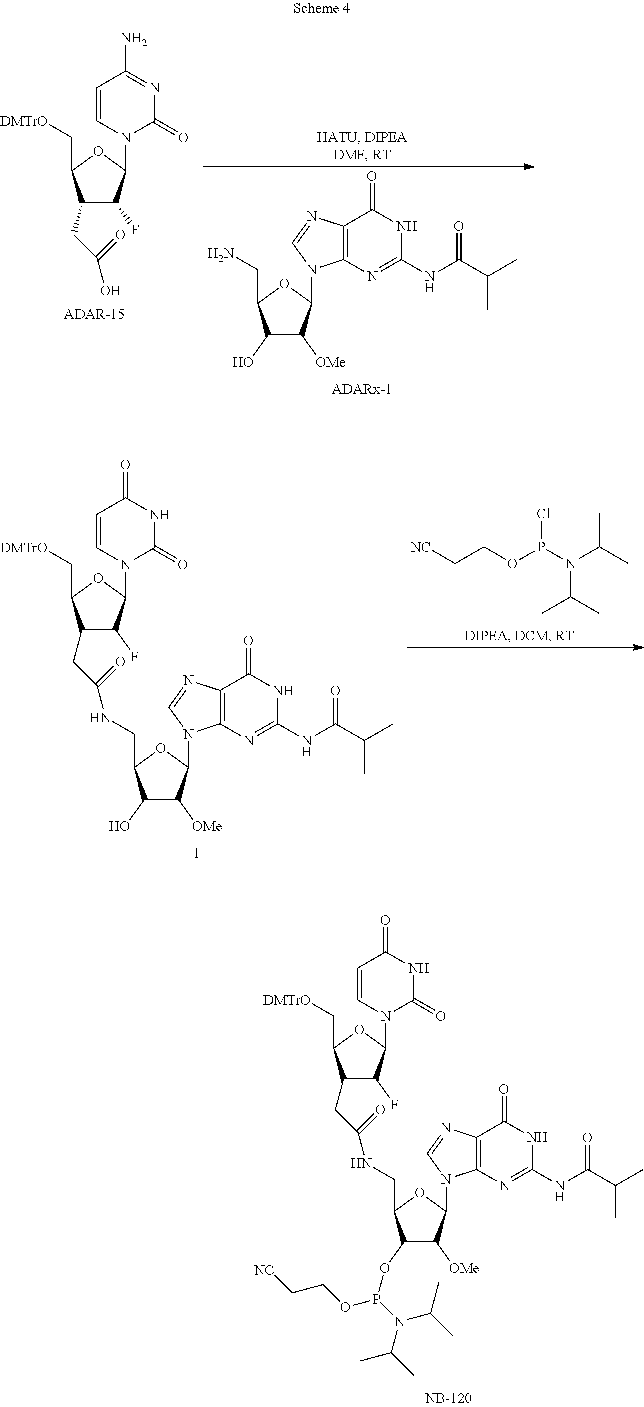

To a stirred solution of alcohol 1 (0.880 g, 0.938 mmol, 1 eq) and diisopropylethylamine (0.979 mL, 5.629 mmol, 6 eq), in DCM (10 mL), was added N, N-diisopropyl chlorophosphoramidite (0.626 mL 2.814 mmol, 3 eq) dropwise. The reaction mixture was stirred at room temperature for 2 h. Reaction mixture was quenched with Aq. Saturated NaHCO₃ solution (30 mL), extracted with DCM (50) ml, washed with brine (50 mL), dried over Na₂SO₄, and evaporated the crude product was loaded on to (pre-equilibrated two times with 1% Et₃N-DCM) Biotage silica gel column (25 g 20 m), and purified by flash chromatography using 0-10% MeOH/DCM containing 1% Et₃N as an additive. Pure fractions were combined and concentrated, dried under high vacuum to obtain Phosphoramidite NB-120 (750 mg, 70%) product as a white solid. 96% purity by HPLC, Mass (m/z 1140 M+1). ³¹P NMR (202, MHz, DMSO-d6) δ 150.58, 150.17.

Oligonucleotide Comprising Dinucleotide NB-120 has been Synthesized Using the General Procedure Described in Example 1.

### Example 12: NB-121

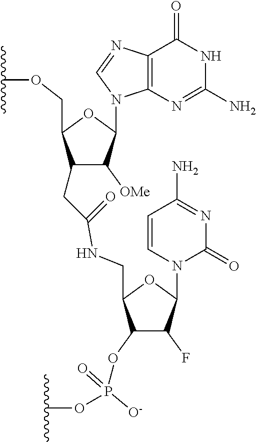

### Synthesis of NB-121

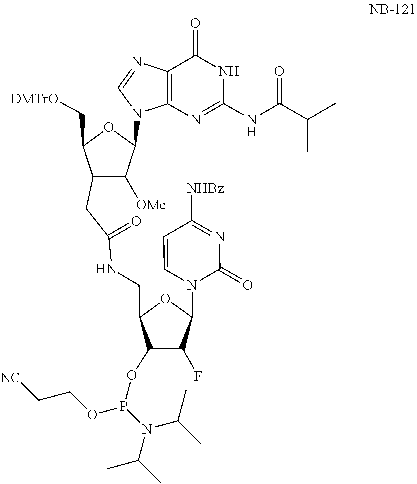

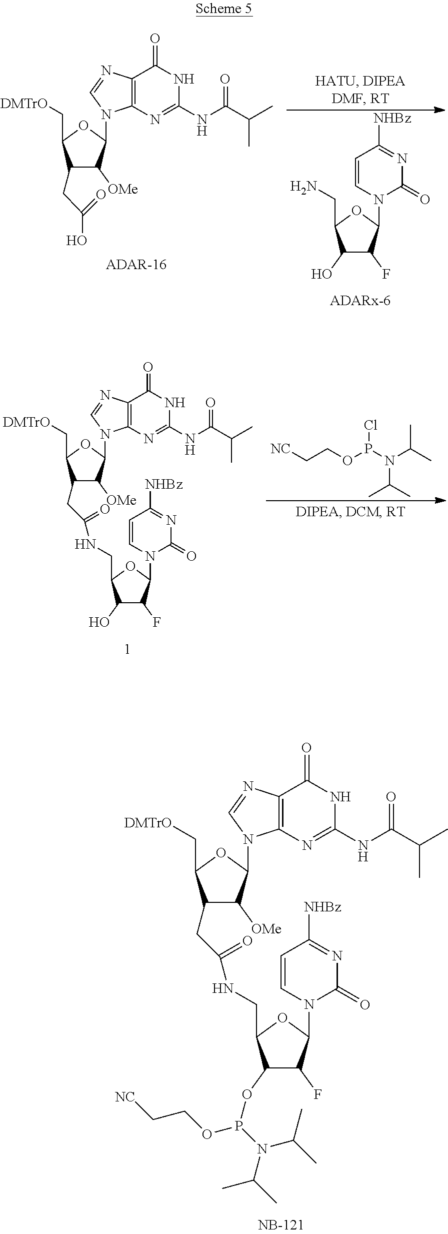

A solution of acid ADAR-16 (0.740 g, 1.041 mmol, 1 eq), DIPEA (0.724 mL, 4.163 mmol, 4 eq) and HATU (0.592 g, 1.561 mmol, 1.5 eq) in DMF (10 mL) was stirred for 15 min at room temperature, then amine ADARx-6 (0.480 g, 1.249 mmol, 1.2 eq) was added and the mixture was stirred for 3 h at room temperature, reaction mixture was diluted with water (50 ml), precipitated solids were filtered and washed with water, the solids were dried and re dissolved in DCM then purified by Ethyl acetate/MeOH, 0-5% as an eluent, pure fractions were combined and concentrated to obtain amide 1 (0.72 g, 55%) as a brown solid. LCMS m/z 1065 (M+Na).

To a stirred solution of alcohol 1 (0.705 g, 0.677 mmol, 1 eq) and diisopropylethylamine (0.706 mL, 4.060 mmol, 6 eq), in DCM (10 mL), was added N, N-diisopropyl chlorophosphoramidite (0.45 mL 2.030 mmol, 3 eq) dropwise. The reaction mixture was stirred at room temperature for 2 h. Reaction mixture was quenched with Aq. Saturated NaHCO₃ solution (30 mL), extracted with DCM (50) ml, washed with brine (50 mL), dried over Na₂SO₄, and evaporated the crude product was loaded on to (pre-equilibrated two times with 1% Et₃N-DCM) Biotage silica gel column (25 g 20 m), and purified by flash chromatography using 0-10% MeOH/DCM containing 1% Et₃N as an additive. Pure fractions were combined and concentrated, dried under high vacuum to obtain Phosphoramidite NB-121 (680 mg, 81%) product as a white solid. 96% purity by HPLC, Mass (m/z 1264 M+Na), ³¹P NMR (202, MHz, DMSO-d6) δ 150.64, 150.62, 150.53.

Oligonucleotide Comprising Dinucleotide NB-121 has been Synthesized Using the General Procedure Described in Example 1.

### Example 13: NB-122

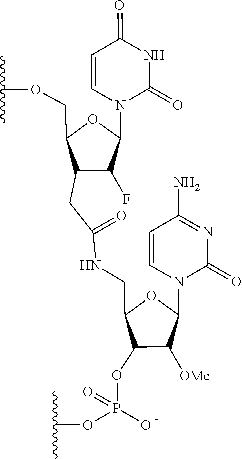

### Synthesis of NB-122

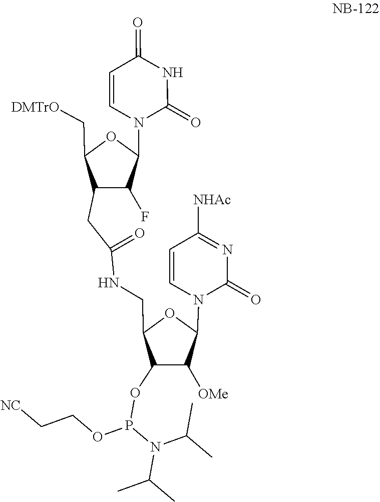

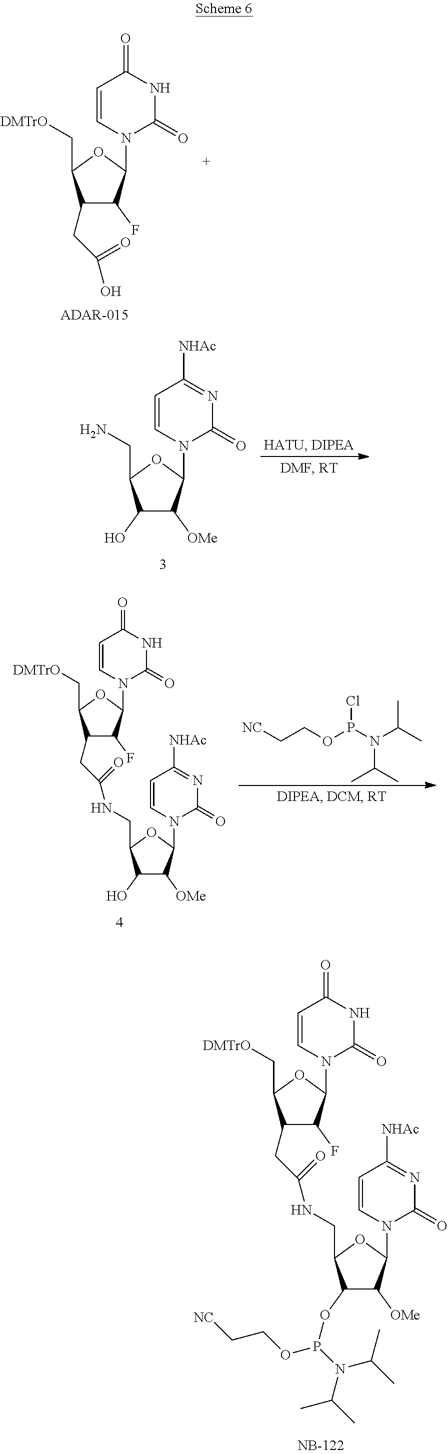

A solution of acid ADAR-015 (0.9 g, 1.525 mmol, 1 eq), DIPEA (0.796 mL, 4.576 mmol, 3 eq) and HATU (0.869 g, 2.288 mmol, 1.5 eq) in DMF (10 mL) was stirred for 15 min at room temperature, then amine 3 (0.500 g, 1.678 mmol, 1.1 eq) was added and the mixture was stirred for 3 h at room temperature, reaction mixture was diluted with water (50 ml), precipitated solids were filtered and washed with water, the solids were dried and re dissolved in DCM then purified by Ethyl acetate/MeOH, 0-5% as an eluent, pure fractions were combined and concentrated to obtain amide 4 (0.75 g, 51%) as a brown solid. LCMS m/z 892 (M+Na).

To a stirred solution of alcohol 4 (0.711 g, 0.817 mmol, 1 eq) and diisopropylethylamine (0.852 mL, 4.903 mmol, 6 eq), in DCM (10 mL), was added N, N-diisopropyl chlorophosphoramidite (0.545 mL 2.452 mmol, 3 eq) dropwise. The reaction mixture was stirred at room temperature for 2 h. Reaction mixture was quenched with Aq. Saturated NaHCO₃ solution (30 mL), extracted with DCM (50) ml, washed with brine (50 mL), dried over Na₂SO₄, and evaporated the crude product was loaded on to (pre-equilibrated two times with 1% Et₃N-DCM) Biotage silica gel column (25 g 20 μm), and purified by flash chromatography using 0-10% MeOH/DCM containing 1% Et₃N as an additive. Pure fractions were combined and concentrated, dried under high vacuum to obtain Phosphoramidite NB-122 (820 mg, 94%) as an off white solid. 98% purity by HPLC, Mass (m/z 1093 M+Na), ³¹P NMR (202, MHz, DMSO-d6) δ 149.99, 149-81.

Oligonucleotide Comprising Dinucleotide NB-122 has been Synthesized Using the General Procedure Described in Example 1.

### Example 14: NB-124

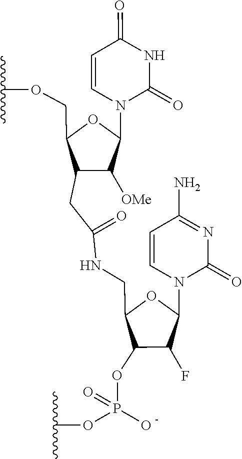

### Synthesis of NB-124

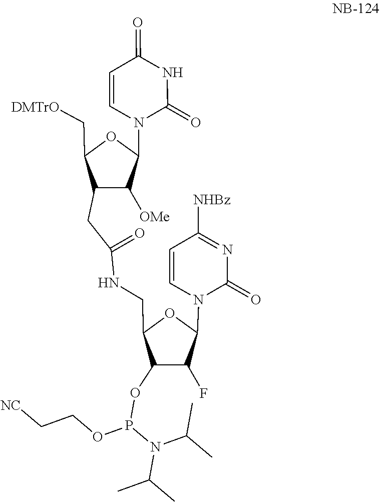

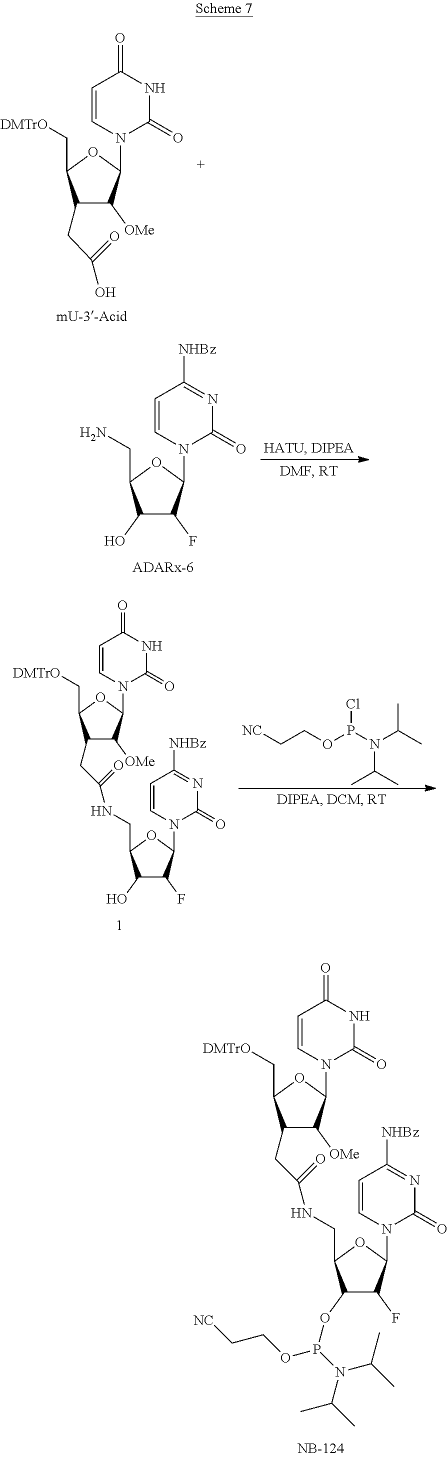

A solution of mU-3′-acid (1 g, 1.661 mmol, 1 eq), DIPEA (0.866 mL, 4.983 mmol, 3 eq) and HATU (0.947 g, 2.49 mmol, 1.5 eq) in DMF (20 mL) was stirred for 15 min at room temperature, then amine ADARx-6 (0.822 g, 1.66 mmol, 1 eq) was added and the mixture was stirred for 3 h at room temperature, reaction mixture was diluted with water (50 ml), precipitated solids were filtered and washed with water, the solids were dried and re dissolved in DCM then purified by Ethyl acetate/MeOH, 0-5% as an eluent, pure fractions were combined and concentrated to obtain amide 1 (0.95 g, 61%) as a brown solid. LCMS m/z 933 (M+1).

To a stirred solution of alcohol 1 (0.910 g, 0.976 mmol, 1 eq) and diisopropylethylamine (1.019 mL, 5.858 mmol, 6 eq), in DCM (10 mL), was added N, N-diisopropyl chlorophosphoramidite (0.65 mL 2.929 mmol, 3 eq) dropwise. The reaction mixture was stirred at room temperature for 2 h. Reaction mixture was quenched with Aq. Saturated NaHCO₃ solution (30 mL), extracted with DCM (50) ml, washed with brine (50 mL), dried over Na₂SO₄, and evaporated the crude product was loaded on to (pre-equilibrated two times with 1% Et₃N-DCM) Biotage silica gel column (25 g 20 μm), and purified by flash chromatography using 0-10% MeOH/DCM containing 1% Et₃N as an additive. Pure fractions were combined and concentrated, dried under high vacuum to obtain Phosphoramidite NB-124 (710 mg, 64%) as an off-white solid. 93% purity by HPLC, Mass (m/z 1155 M+Na), ³¹P NMR (202, MHz, DMSO-d6) δ 150.62.

Oligonucleotide Comprising Dinucleotide NB-124 has been Synthesized Using the General Procedure Described in Example 1.

### Example 15: NB-125

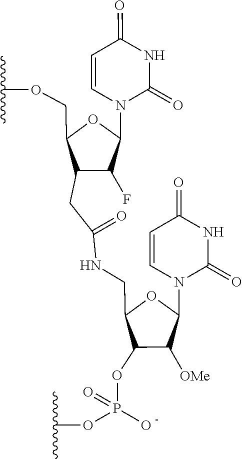

### Synthesis of NB-125

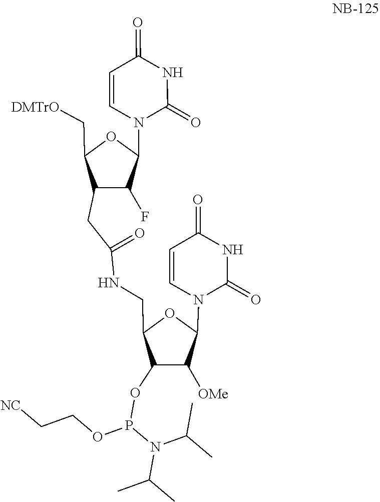

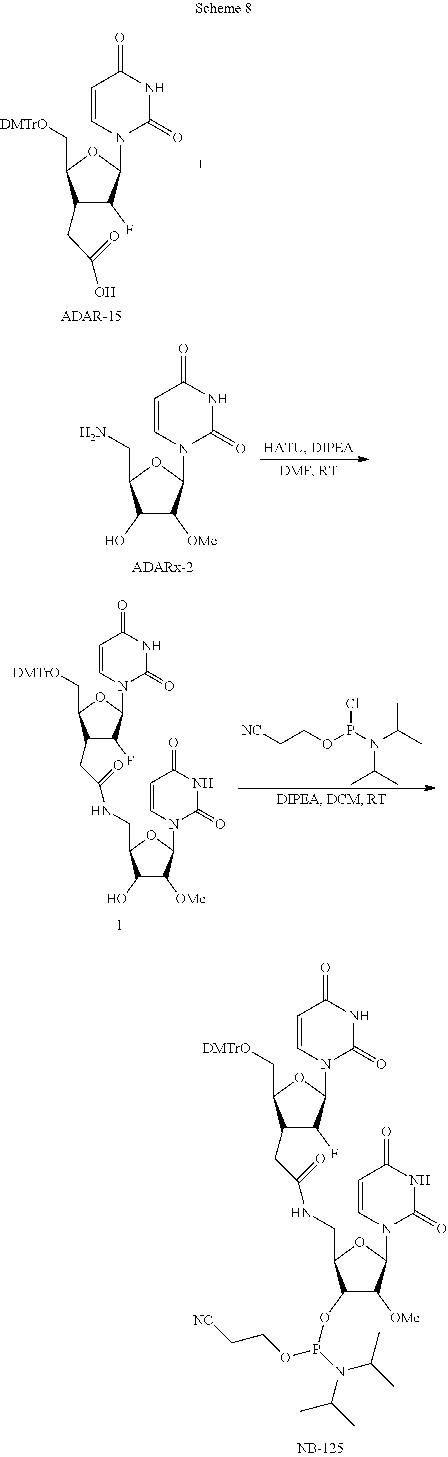

A solution of acid ADAR-15 (1 g, 1.695 mmol, 1 eq), DIPEA (0.884 mL, 5.085 mmol, 3 eq) and HATU (0.966 g, 2.54 mmol, 1.5 eq) in DMF (10 mL) was stirred for 15 min at room temperature, then amine ADARx-2 (0.479 g, 1.864 mmol, 1.1 eq) was added and the mixture was stirred for 3 h at room temperature, reaction mixture was diluted with water (50 ml), precipitated solids were filtered and washed with water, the solids were dried and re dissolved in DCM then purified by Ethyl acetate/MeOH, 0-5% as an eluent, pure fractions were combined and concentrated to obtain amide (1.1 g, 56%) as a brown solid. LCMS m/z 830 (M+1).

To a stirred solution of alcohol 1 (1.090 g, 01.315 mmol, 1 eq) and diisopropylethylamine (1.37 mL, 7.889 mmol, 6 eq), in DCM (10 mL), was added N, N-diisopropyl chlorophosphoramidite (0.87 mL 3.945 mmol, 3 eq) dropwise. The reaction mixture was stirred at room temperature for 2 h. Reaction mixture was quenched with Aq. Saturated NaHCO₃ solution (30 mL), extracted with DCM (50) ml, washed with brine (50 mL), dried over Na₂SO₄, and evaporated the crude product was loaded on to (pre-equilibrated two times with 1% Et₃N-DCM) Biotage silica gel column (25 g 20 μm), and purified by flash chromatography using 0-10% MeOH/DCM containing 1% Et₃N as an additive. Pure fractions were combined and concentrated, dried under high vacuum to obtain Phosphoramidite NB-125 (950 mg, 70%) product as a beige solid. 98% purity by HPLC, Mass (m/z 1052 M+Na), ³¹P NMR (202, MHz, DMSO-d6) δ 150.08, 149.63.

Oligonucleotide Comprising Dinucleotide NB-125 has been Synthesized Using the General Procedure Described in Example 1.

### Example 16: NB-126

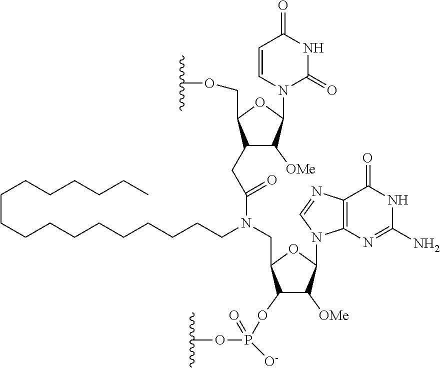

### Synthesis of NB-126

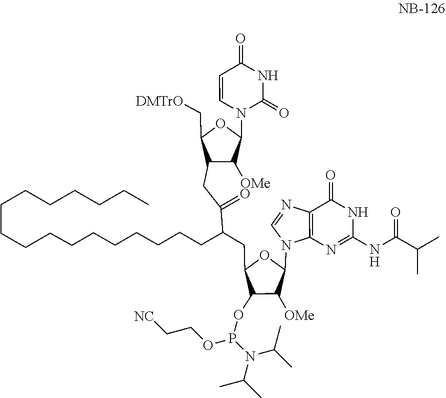

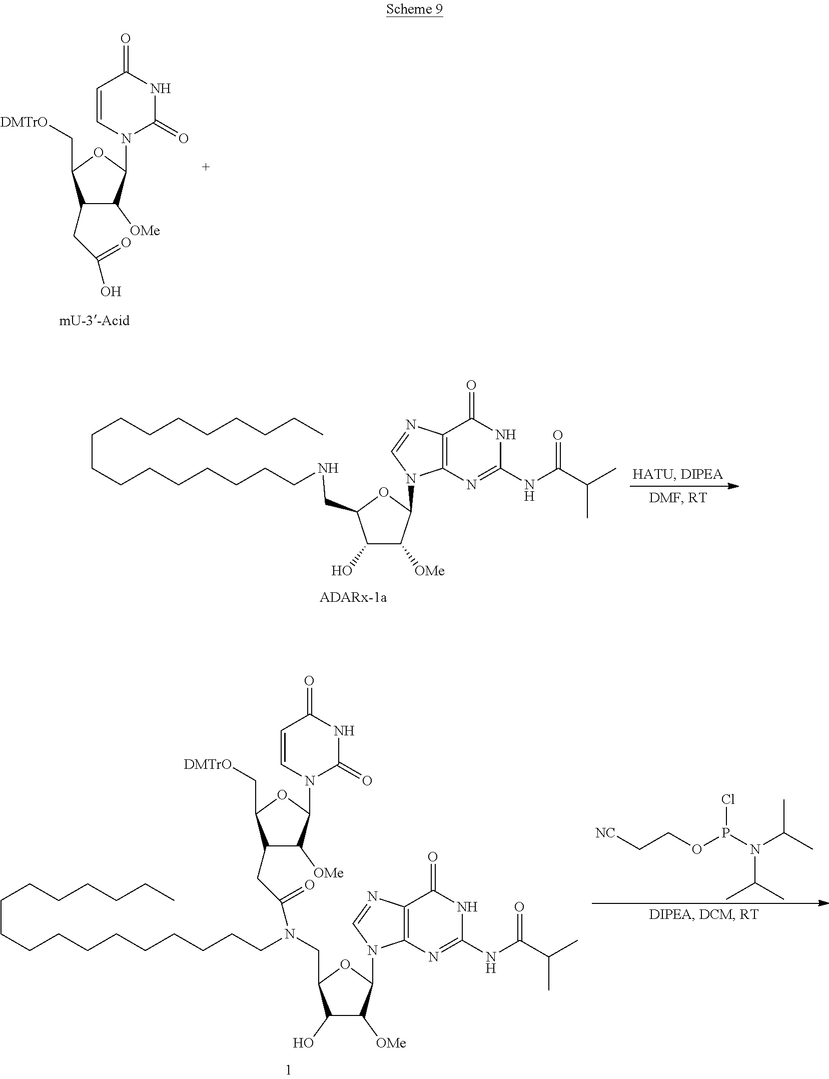

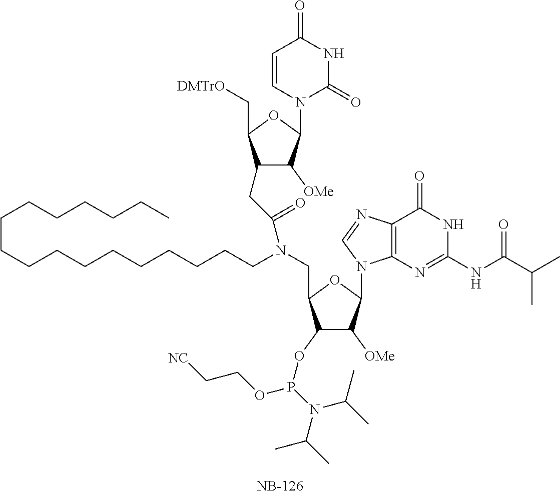

A solution of acid mU-3′-Acid (0.65 g, 1.080 mmol, 1 eq), DIPEA (0.563 mL, 3.239 mmol, 3 eq) and HATU (0.615 g, 1.62 mmol, 1.5 eq) in DMF (20 mL) was stirred for 15 min at room temperature, then amine ADARx-1a (0.652 g, 1.08 mmol, 1 eq) was added and the mixture was stirred for 3 h at room temperature, reaction mixture was diluted with water (50 ml), precipitated solids were filtered and washed with water, the solids were dried and re dissolved in DCM then purified by Ethyl acetate/MeOH, 0-10% as an eluent, pure fractions were combined and concentrated to obtain amide 1 (0.94 g, 73%) as a brown solid. LCMS m/z 1190 (M+1).

To a stirred solution of alcohol 1 (0.93 g, 0.782 mmol, 1 eq) and diisopropylethylamine (0.816 mL, 4.693 mmol, 6 eq), in DCM (10 mL), was added N, N-diisopropyl chlorophosphoramidite (0.522 mL, 2.347 mmol, 3 eq) dropwise. The reaction mixture was stirred at room temperature for 5 h. Reaction mixture was quenched with Aq. Saturated NaHCO₃ solution (30 mL), extracted with DCM (50) ml, washed with brine (50 mL), dried over Na₂SO₄, and evaporated the crude product was loaded on to (pre-equilibrated two times with 1% Et₃N-DCM) Biotage silica gel column (25 g 20 μm), and purified by flash chromatography using 0-10% MeOH/DCM containing 1% Et₃N as an additive. Pure fractions were combined and concentrated, dried under high vacuum to obtain Phosphoramidite NB-126 (910 mg, 84%) as a beige solid. 98% purity by HPLC, Mass (m/z 1413 M+Na). ³¹p NMR (202, MHz, DMSO-d6) δ 150.46, 150.31, 150.00.

Oligonucleotide Comprising Dinucleotide NB-126 has been Synthesized Using the General Procedure Described in Example 1.

### Example 17: NB-127

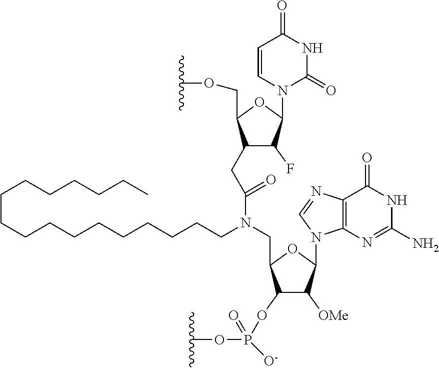

### Synthesis of NB-127

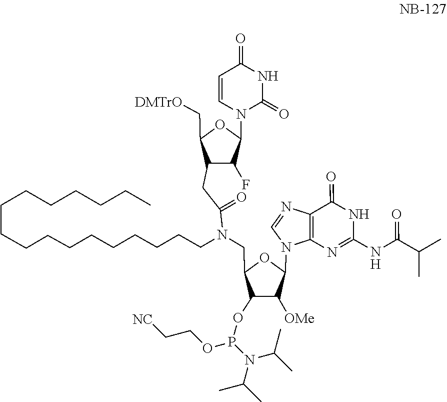

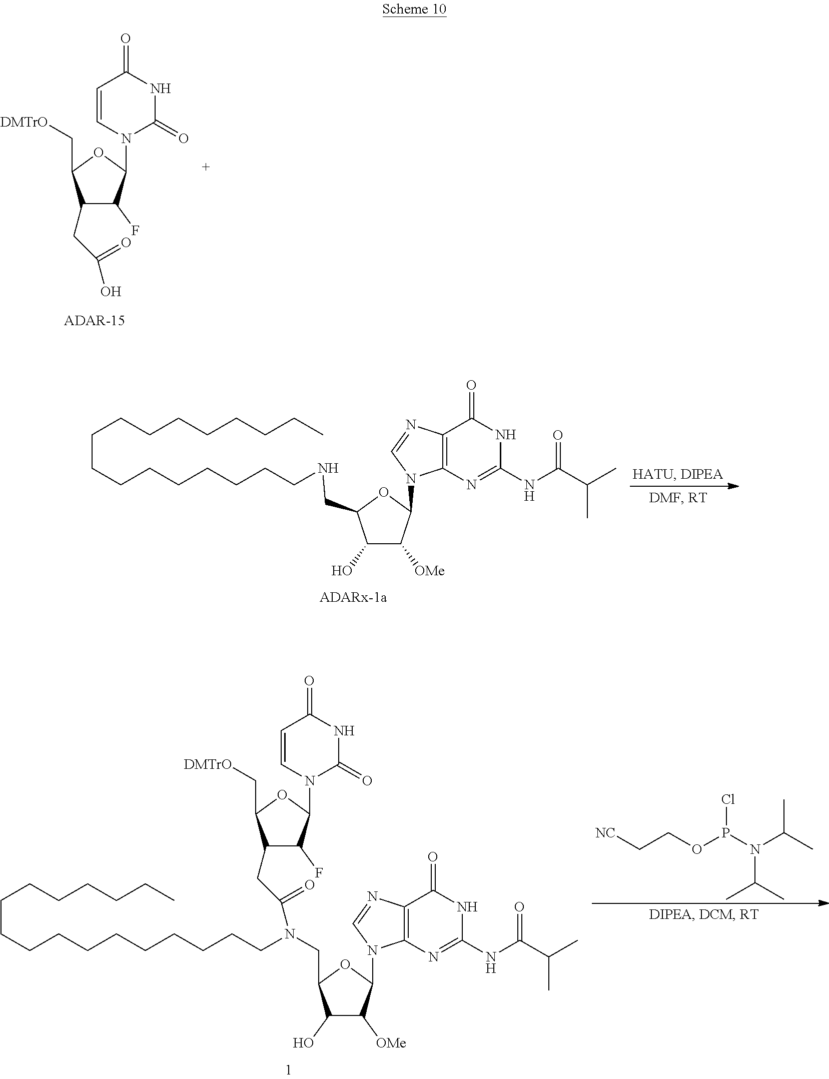

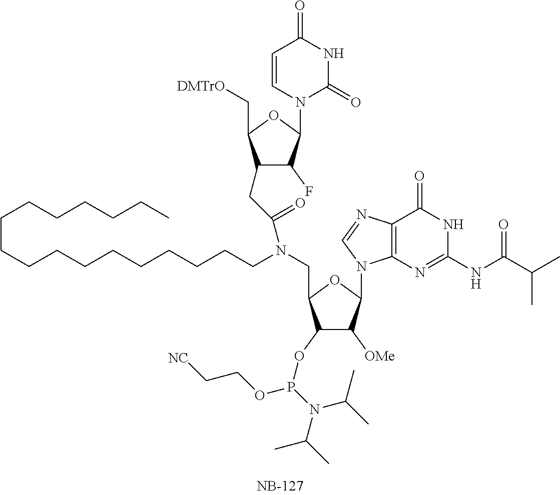

A solution of acid ADAR-15 (0.65 g, 1.102 mmol, 1 eq), DIPEA (0.575 mL, 3.305 mmol, 3 eq) and HATU (0.628 g, 1.65 mmol, 1.5 eq) in DMF (10 mL) was stirred for 15 min at room temperature, then amine ADARx-1a (0.665 g, 1.102 mmol, 1.0 eq) was added and the mixture was stirred for 3 h at room temperature, reaction mixture was diluted with water (50 ml), precipitated solids were filtered and washed with water, the solids were dried and re dissolved in DCM then purified by Ethyl acetate/MeOH, 0-5% as an eluent, pure fractions were combined and concentrated to obtain amide 1 (0.93 g, 72%) as a brown solid. LCMS m/z 1199 (M+Na).

To a stirred solution of alcohol 1 (0.91 g, 0.773 mmol, 1 eq) and diisopropylethylamine (0.806 mL, 4.639 mmol, 6 eq), in DCM (10 mL), was added N, N-diisopropyl chlorophosphoramidite (0.516 mL 2.319 mmol, 3 eq) dropwise. The reaction mixture was stirred at room temperature for 2 h. Reaction mixture was quenched with Aq. Saturated NaHCO₃ solution (30 mL), extracted with DCM (50) ml, washed with brine (50 mL), dried over Na₂SO₄, and evaporated the crude product was loaded on to (pre-equilibrated two times with 1% Et₃N-DCM) Biotage silica gel column (25 g 20 μm), and purified by flash chromatography using 0-10% MeOH/DCM containing 1% Et₃N as an additive. Pure fractions were combined and concentrated, dried under high vacuum to obtain Phosphoramidite NB-127 (920 mg, 86%) as a beige solid. 98% purity by HPLC, Mass (m/z 1399 M+Na), ³¹P NMR (202, MHz, DMSO-d6) δ 150.54, 150.50, 150.36, 149.97.

Oligonucleotide Comprising Dinucleotide NB-127 has been Synthesized Using the General Procedure Described in Example 1.

### Example 18: NB-128

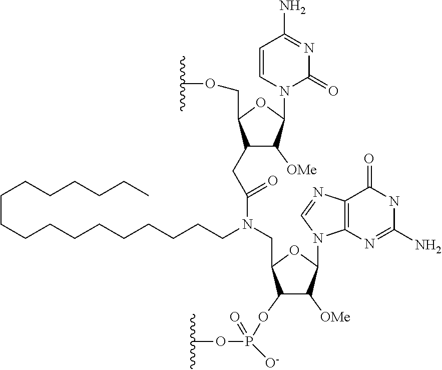

### Synthesis of NB-128

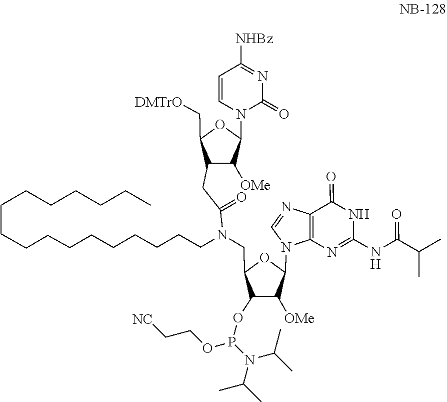

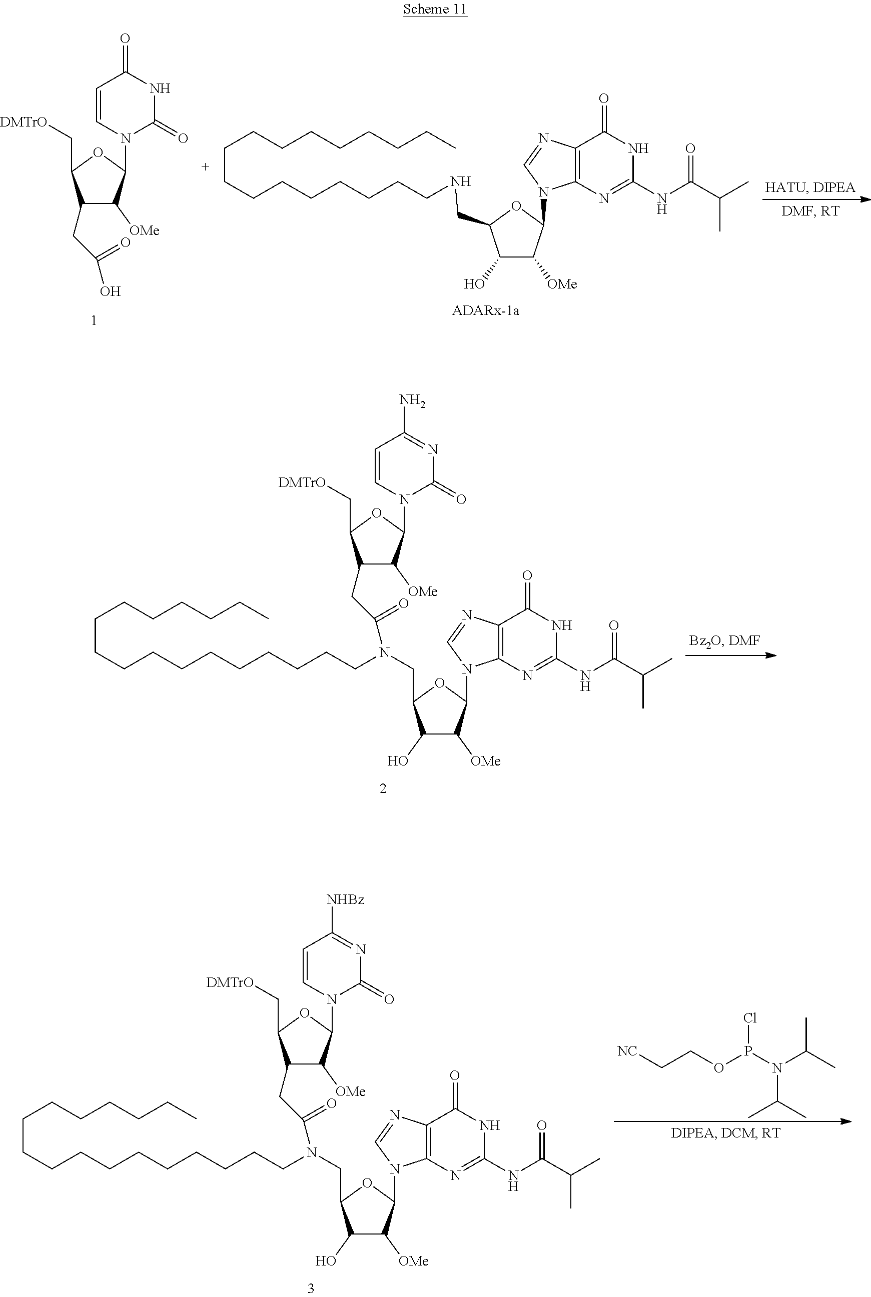

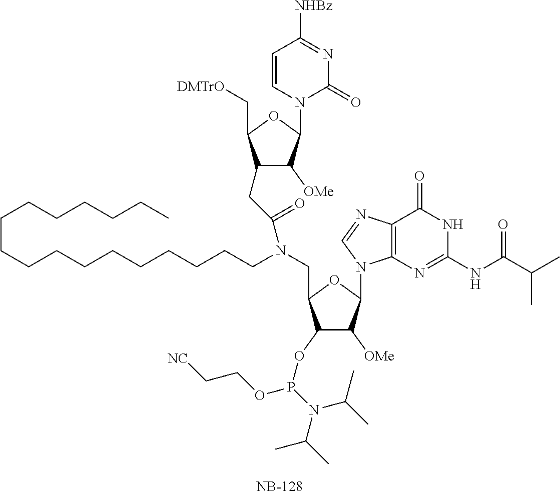

A solution of acid 1 (0.4 g, 0.666 mmol, 1 eq), DIPEA (0.347 mL, 1.997 mmol, 3 eq) and HATU (0.379 g, 0.998 mmol, 1.5 eq) in DMF (10 mL) was stirred for 15 min at room temperature, then amine ADARx-1a (0.402 g, 0.666 mmol, 1.0 eq) was added and the mixture was stirred for 3 h at room temperature, reaction mixture was diluted with water (50 ml), precipitated solids were filtered and washed with water, the solids were dried and re dissolved in DCM then purified by Ethyl acetate/MeOH, 0-5% as an eluent, pure fractions were combined and concentrated to obtain amide 2 (0.51 g, 64%) as a brown solid. LCMS m/z 1189 (M+1).

A mixture of amine 2 (0.5 g, 0.421 mmol, 1 eq) and Benzoic anhydride (105 mg, 0.463 mmol, 1.1 eq) in DMF (10 mL) was stirred overnight at RT. The reaction mixture was diluted with DCM 100 mL, the organic phase washed with aq. NaCl solution 50 mL, dried over Na₂SO₄, and concentrated in vacuo. The residue was purified by flash chromatography on silica gel. using MeOH/EtOAc. 0-10% as an fluent. Pure fractions were combined and concentrated to obtain amide 3 (490 mg, 90%) as a white solid. LCMS m/z 1314 (M+Na).

To a stirred solution of alcohol 3 (0.485 g, 0.375 mmol, 1 eq) and diisopropylethylamine (0.392 mL, 2.252 mmol, 6 eq), in DCM (10 mL), was added N, N-diisopropyl chlorophosphoramidite (0.25 mL 1.126 mmol, 3 eq) dropwise. The reaction mixture was stirred at room temperature for 4 h. Reaction mixture was quenched with Aq. Saturated NaHCO₃ solution (30 mL), extracted with DCM (50) ml, washed with brine (50 mL), dried over Na₂SO₄, and evaporated the crude product was loaded on to (pre-equilibrated two times with 1% Et₃N-DCM) Biotage silica gel column (25 g 20 μm), and purified by flash chromatography using 0-10% MeOH/DCM containing 1% Et₃N as an additive. Pure fractions were combined and concentrated, dried under high vacuum to obtain Phosphoramidite NB-128 (250 mg, 44%) as a beige solid. 98% purity by HPLC, Mass (m/z 1514 M+Na), ³¹P NMR (202, MHz, DMSO-d6) δ 150.52, 150.31, 150.12.

Oligonucleotide Comprising Dinucleotide NB-128 has been Synthesized Using the General Procedure Described in Example 1.

### Example 19: NB-129

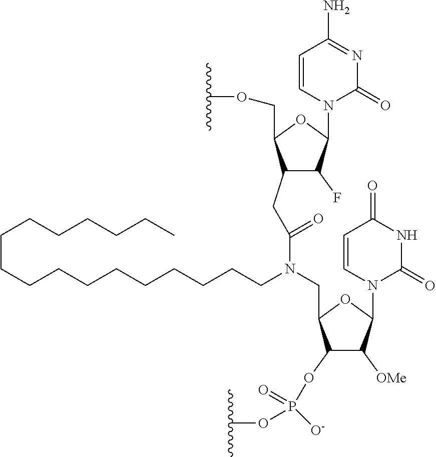

### Synthesis of NB-129

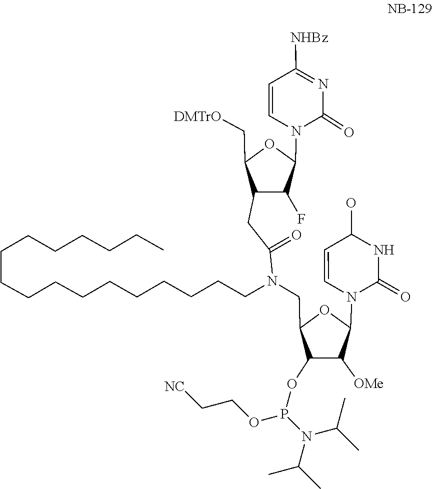

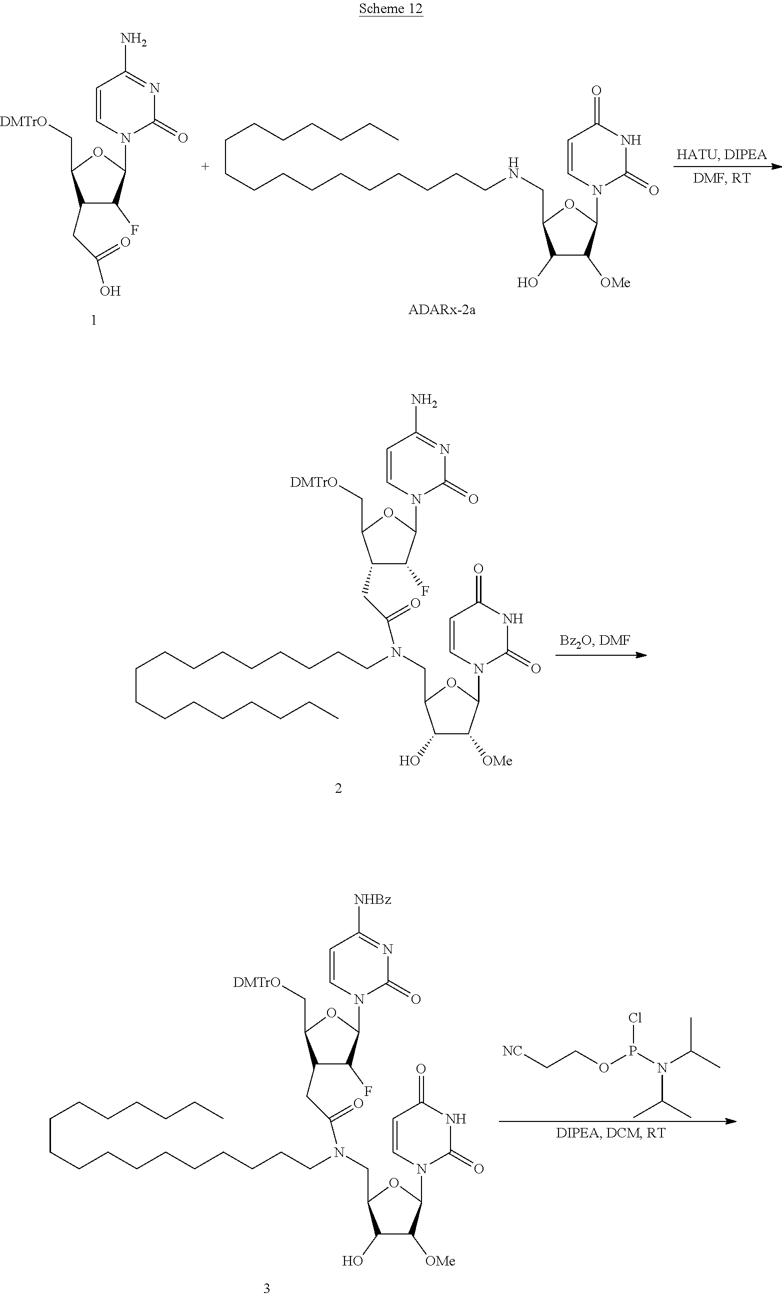

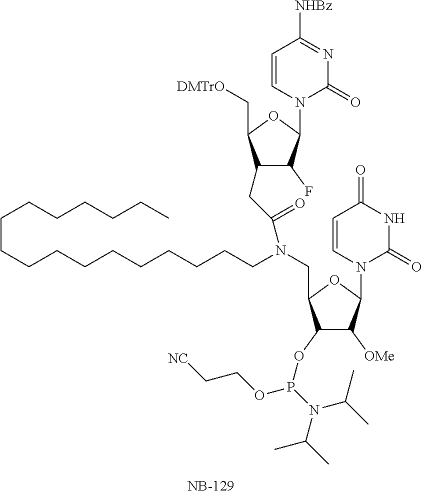

A solution of acid 1 (0.900 g, 1.528 mmol, 1 eq), DIPEA (0.8 mL, 4.6 mmol, 3 eq) and HATU (0.871 g, 2.292 mmol, 1.5 eq) in DMF (20 mL) was stirred for 15 min at room temperature, then amine ADARx-2a (0.756 g, 1.528 mmol, 1.0 eq) was added and the mixture was stirred for 12 h at room temperature, reaction mixture was diluted with water (50 ml), precipitated solids were filtered and washed with water, the solids were dried and re dissolved in DCM then purified by Ethyl acetate/MeOH, 0-10% as an eluent, pure fractions were combined and concentrated to obtain amide 2 (0.98 g, 60%) as a white solid. LCMS m/z 1089 (M+Na).

A mixture of amine 2 (0.970 g, 0.909 mmol, 1 eq) and Benzoic anhydride (226 mg, 1 mmol, 1.1 eq) in DMF (10 mL) was stirred overnight at RT. The reaction mixture was diluted with DCM 100 mL, the organic phase washed with aq. NaCl solution 50 mL, dried over Na₂SO₄, and concentrated in vacuo. The residue was purified by flash chromatography on silica gel. using MeOH/EtOAc. 0-10% as an eluent. Pure fractions were combined and concentrated to obtain amide 3 (850 mg, 80%) as a white solid. LCMS m/z 1172 (M+1).

To a stirred solution of alcohol 3 (0.6 g, 0.512 mmol, 1 eq) and diisopropylethylamine (0.534 mL, 3.074 mmol, 6 eq), in DCM (10 mL), was added N, N-diisopropyl chlorophosphoramidite (0.342 mL 3.945 mmol, 3 eq) dropwise. The reaction mixture was stirred at room temperature for 3 h. Reaction mixture was quenched with Aq. Saturated NaHCO₃ solution (30 mL), extracted with DCM (50) ml, washed with brine (50 mL), dried over Na₂SO₄, and evaporated the crude product was loaded on to (pre-equilibrated with 1% Et₃N-DCM) Biotage silica gel column (25 g 20 μm), and purified by flash chromatography using 0-10% MeOH/DCM containing 1% Et₃N as an additive. Pure fractions were combined and concentrated, dried under high vacuum to obtain Phosphoramidite NB-129 (250 mg, 35%) as a beige solid. 95% purity by HPLC. Mass (m/z 1394 M+Na) corresponds with product. ³¹P NMR (202, MHz, DMSO-d6) δ 149.80, 149.74, 149.55, 149.50.

Oligonucleotide Comprising Dinucleotide NB-129 has been Synthesized Using the General Procedure Described in Example 1.

### Example 20: NB-130

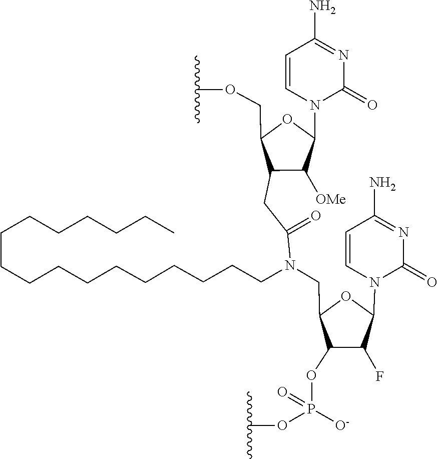

### Synthesis of NB-130

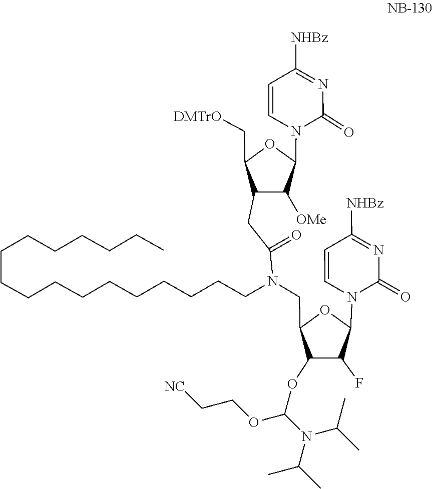

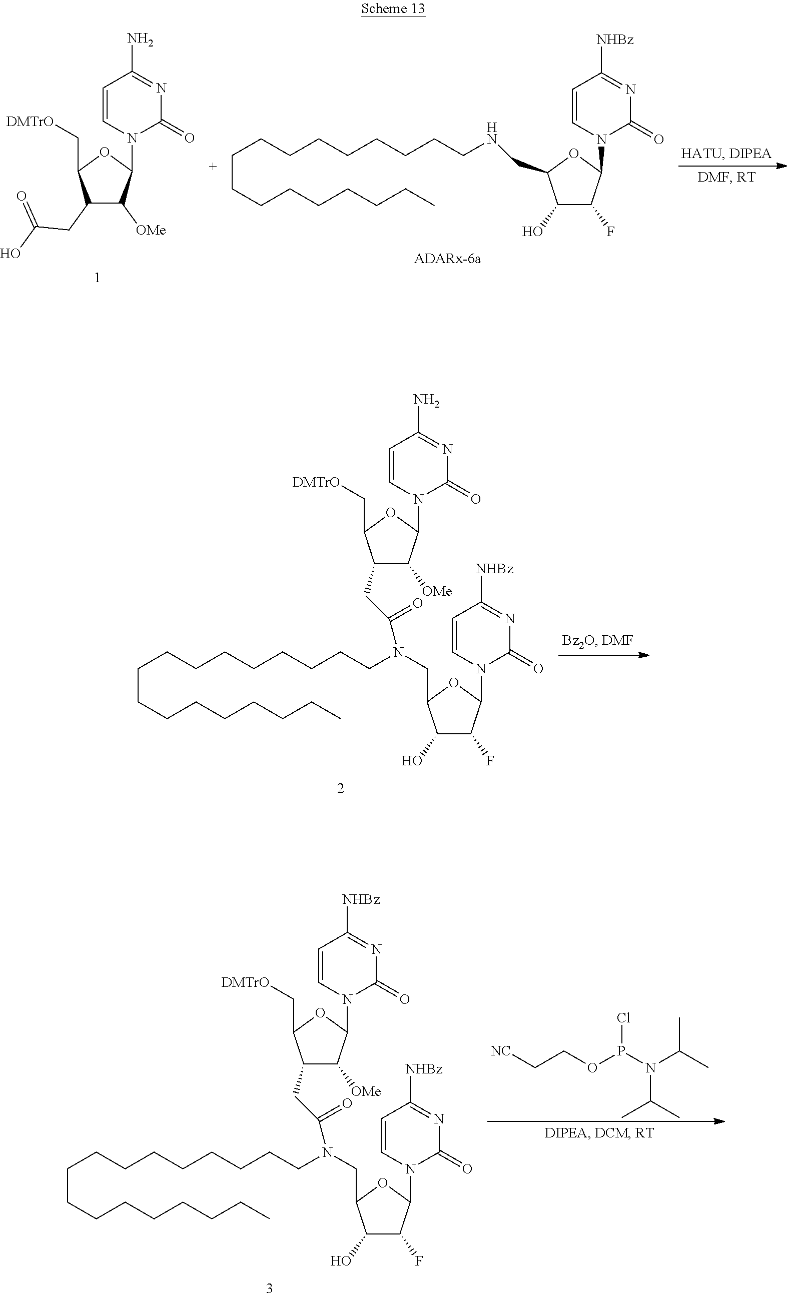

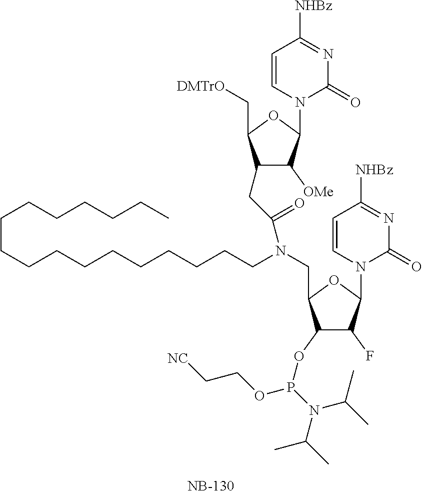

A solution of acid 1 (1.090 g, 1.814 mmol, 1 eq), DIPEA (0.946 mL, 5.441 mmol, 3 eq) and HATU (1.034 g, 2.720 mmol, 1.5 eq) in DMF (20 mL) was stirred for 15 min at room temperature, then amine ADARx-6a (1.063 g, 1.814 mmol, 1.0 eq) was added and the mixture was stirred for 12 h at room temperature, reaction mixture was diluted with water (50 ml), precipitated solids were filtered and washed with water, the solids were dried and re dissolved in DCM then purified by Ethyl acetate/MeOH, 0-10% as an eluent, pure fractions were combined and concentrated to obtain amide 2 (1.5 g, 70%) as a white solid. NMR and LCMS m/z 1192 (M+Na).

A mixture of amine 2 (1.5 g, 1.282 mmol, 1 eq) and Benzoic anhydride (435 mg, 1.923 mmol, 1.5 eq) in DMF (20 mL) was stirred overnight at RT. The reaction mixture was diluted with DCM 100 mL, the organic phase washed with aq. NaCl solution 50 mL, dried over Na₂SO₄, and concentrated in vacuo. The residue was purified by flash chromatography on silica gel. using MeOH/EtOAc. 0-10% as an eluent. Pure fractions were combined and concentrated to obtain amide 3 (840 mg, 51%) as a white solid. LCMS m/z 1275 (M+1).

To a stirred solution of alcohol 3 (0.810 g, 0.636 mmol, 1 eq) and diisopropylethylamine (0.663 mL, 3.815 mmol, 6 eq), in DCM (10 mL), was added N, N-diisopropyl chlorophosphoramidite (0.424 mL 1.907 mmol, 3 eq) dropwise. The reaction mixture was stirred at room temperature for 12 h. Reaction mixture was quenched with Aq. Saturated NaHCO₃ solution (50 mL), extracted with DCM (100) ml, washed with brine (100 mL), dried over Na₂SO₄, and evaporated the crude product was loaded on to (pre-equilibrated with 1% Et₃N-DCM) Biotage silica gel column (25 g 20 μm), and purified using 0-10% MeOH/DCM containing 1% Et₃N as an additive. Pure fractions were combined and concentrated, dried under high vacuum to obtain Phosphoramidite NB-130 (650 mg, 69%) as a beige solid. 90% purity by HPLC. Mass (m/z 1496 M+Na). ³¹P NMR (202, MHz, DMSO-d6) δ 150.70, 150.52, 150.27, 149.97.

Oligonucleotide Comprising Dinucleotide NB-130 has been Synthesized Using the General Procedure Described in Example 1.

### Example 21: NB-131

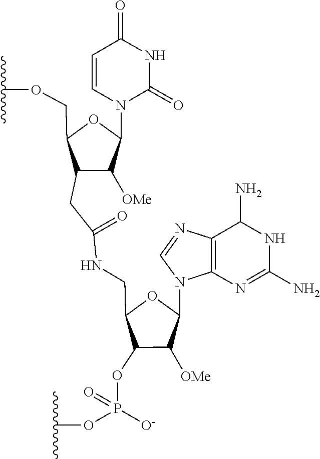

### Synthesis of NB-131

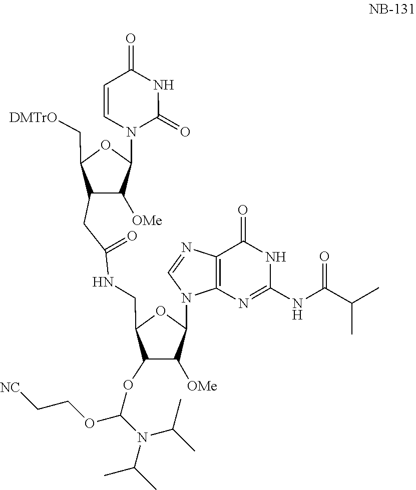

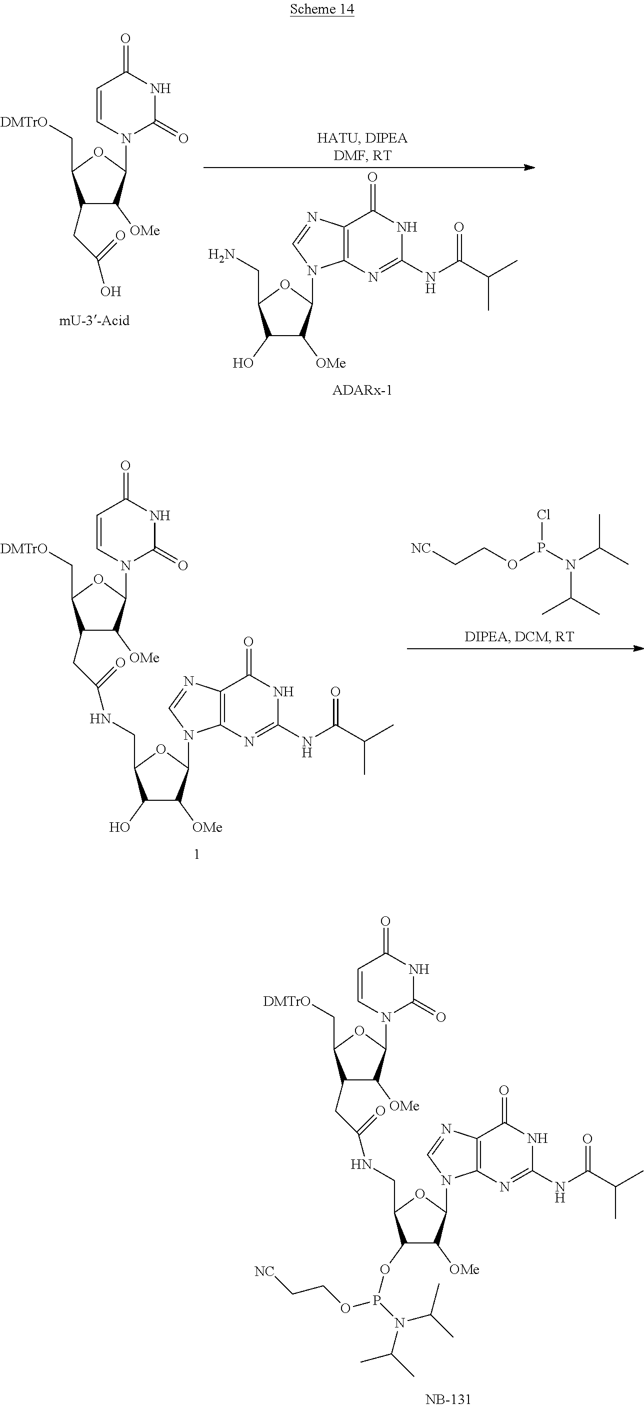

A solution of mU-3′-Acid (1.5 g, 2.49 mmol, 1 eq), DIPEA (1.3 mL, 7.475 mmol, 3 eq) HATU (1.42 g, 3.738 mmol, 1.5 eq) and amine ADARx-1 (0.912 g, 2.49 mmol, 1 eq) in DMF (20 mL) was stirred for 3 h at room temperature, reaction mixture was diluted with saturated NaCl solution (50 ml), precipitated solids were filtered and washed with water, the solids were dried and re dissolved in DCM then purified by MeOH/DCM 0-20% as an eluent, pure fractions were combined and concentrated to obtain amide 1 (1.39 g, 58%) as a brown solid. LCMS m/z 972 (M+Na).

To a stirred solution of alcohol (1.305 g, 1.378 mmol, 1 eq) and diisopropylethylamine (1.43 mL, 8.233 mmol, 6 eq), in DCM (30 mL), was added N, N-diisopropyl chlorophosphoramidite (0.916 mL 4.117 mmol, 3 eq) dropwise. The reaction mixture was stirred at room temperature for 6 h. Reaction mixture was quenched with Aq. Saturated NaHCO₃ solution (100 mL), extracted with DCM (200) ml, washed with brine (100 mL), dried over Na₂SO₄, and evaporated the crude product was loaded on to (pre-equilibrated with 1% Et₃N-EtOAc) Biotage silica gel column (100 g 20 μm), and purified using 0-20% MeOH/EtOAc containing 1% Et₃N as an additive. Pure fractions were combined and concentrated, dried under high vacuum to obtain Phosphoramidite NB-131 (1.44 g, 91%) product as an off-white solid. 95% purity by LCMS, Mass (m/z 1173 M+Na). ³¹P NMR (202, MHz, DMSO-d6) δ 150.46, 150.20.

Oligonucleotide Comprising Dinucleotide NB-131 has been Synthesized Using the General Procedure Described in Example 1.

### Example 22: NB-132

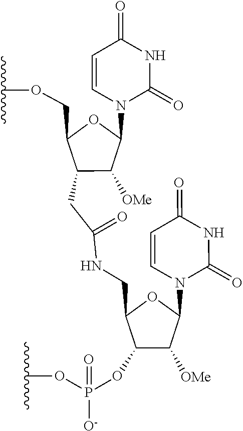 [SMILES: *OC[C@H]1O[C@@H](n2ccc(=O)[nH]c2=O)[C@H](OC)[C@@H]1CC(=O)NC[C@H]1O[C@@H](n2ccc(=O)[nH]c2=O)[C@H](OC)[C@@H]1OP(=O)([O-])O*]

### Synthesis of NB-132: (2R,3R,4R,5R)-2-((2-((2S,3R,4R,5R)-2-((bis(4methoxyphenyl)(phenyl)methoxy)methyl)-5-(2,4-dioxo-3,4-dihydropyrimidin-1(2H)-yl)-4-methoxytetrahydrofuran-3-yl)acetamido) methyl)-5-(2,4-dioxo-3,4-dihydropyrimidin-1(2H)-yl)-4-methoxytetrahydrofuran-3-yl(2-cyanoethyl) diisopropylphosphoramidite

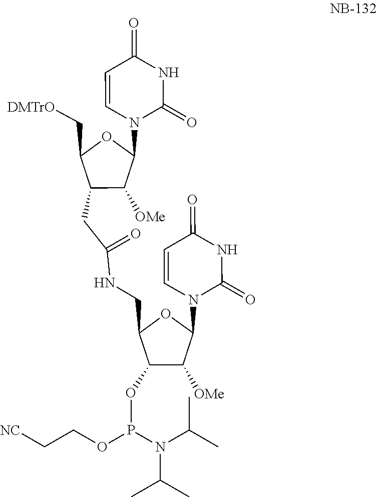

Dinucleotide NB-132 has been synthesized following the procedure described for compound NB-131 (example 21). Mass, 1064.4 [M+Na]⁺, ³¹P NMR (202 MHz, DMSO-d₆) δ 150.01, 149.63.

Oligonucleotide Comprising Dinucleotide NB-132 has been Synthesized Using the General Procedure Described in Example 1.

### Example 23: NB-133

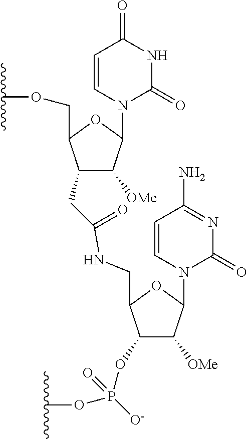 [SMILES: *OCC1OC(n2ccc(=O)[nH]c2=O)[C@H](OC)[C@@H]1CC(=O)NCC1OC(n2ccc(N)nc2=O)[C@H](OC)[C@@H]1OP(=O)([O-])O*]

### Synthesis of NB-133

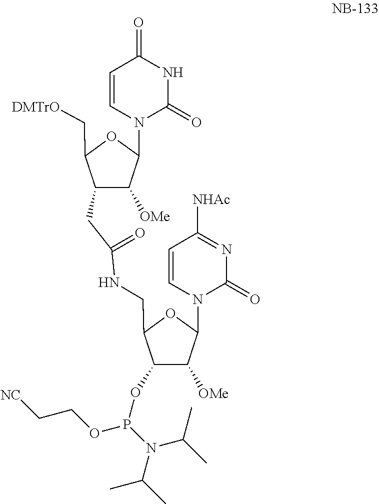

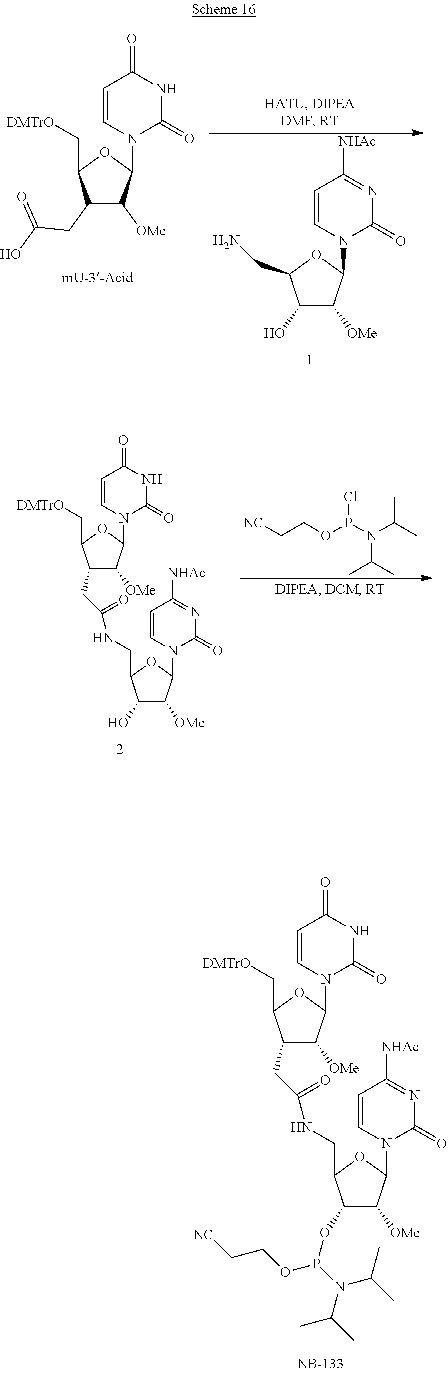

A solution of mU-3′-Acid (0.6 g, 0.997 mmol, 1 eq), DIPEA (0.52 mL, 2.99 mmol, 3 eq), HATU (0.568 g, 1.495 mmol, 1.5 eq) and amine 1 (0.297 g, 0.997 mmol, 1.0 eq) in DMF (10 mL) was stirred for 3 h at room temperature, reaction mixture was diluted with Aq. Sat. NaHCO₃ (50 ml), precipitated solids were filtered and washed with water, the solids were dried and re dissolved in DCM then purified by Ethyl acetate/MeOH, 0-10% as an eluent, pure fractions were combined and concentrated to obtain amide 2 (0.42 g, 48%) as a white solid. LCMS m/z 905 (M+Na).

Synthesis of compound NB-133: To a stirred solution of alcohol 2 (0.395 g, 0.448 mmol, 1 eq) and diisopropylethylamine (0.467 mL, 2.687 mmol, 6 eq), in DCM (10 mL), was added N, N-diisopropyl chlorophosphoramidite (0.3 mL 1.34 mmol, 3 eq) dropwise. The reaction mixture was stirred at room temperature for 2 h. Reaction mixture was quenched with Aq. Saturated NaHCO₃ solution (30 mL), extracted with DCM (50) ml, washed with brine (50 mL), dried over Na₂SO₄, and evaporated the crude product was loaded on to (pre-equilibrated with 1% Et₃N-DCM) Biotage silica gel column (50 g 20 μm), and purified by flash chromatography using MeOH/EtOAc 0-10% 5 CV then 10-10% 10 CV containing 1% Et₃N as an additive. Pure fractions were combined and concentrated, dried under high vacuum to obtain Phosphoramidite NB-133 (430 mg, 87%) as a white solid. 95% purity by HPLC, Mass (m/z 1104 M+Na), ³¹P NMR (202, MHz, DMSO-d6) δ 149.92, 149.79.

Oligonucleotide Comprising Dinucleotide NB-133 has been Synthesized Using the General Procedure Described in Example 1.

### Example 24: NB-134

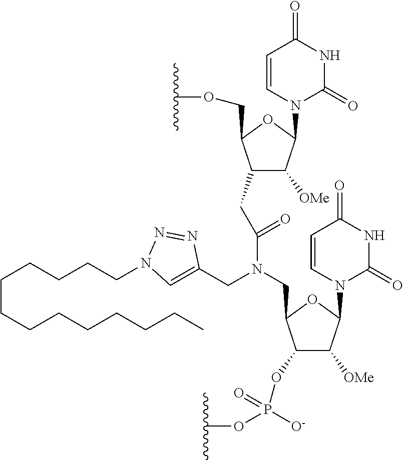

### Synthesis of NB-134

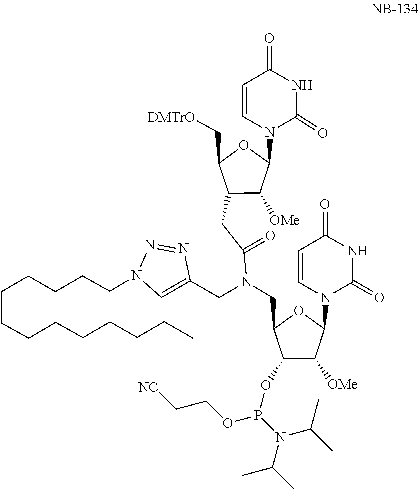

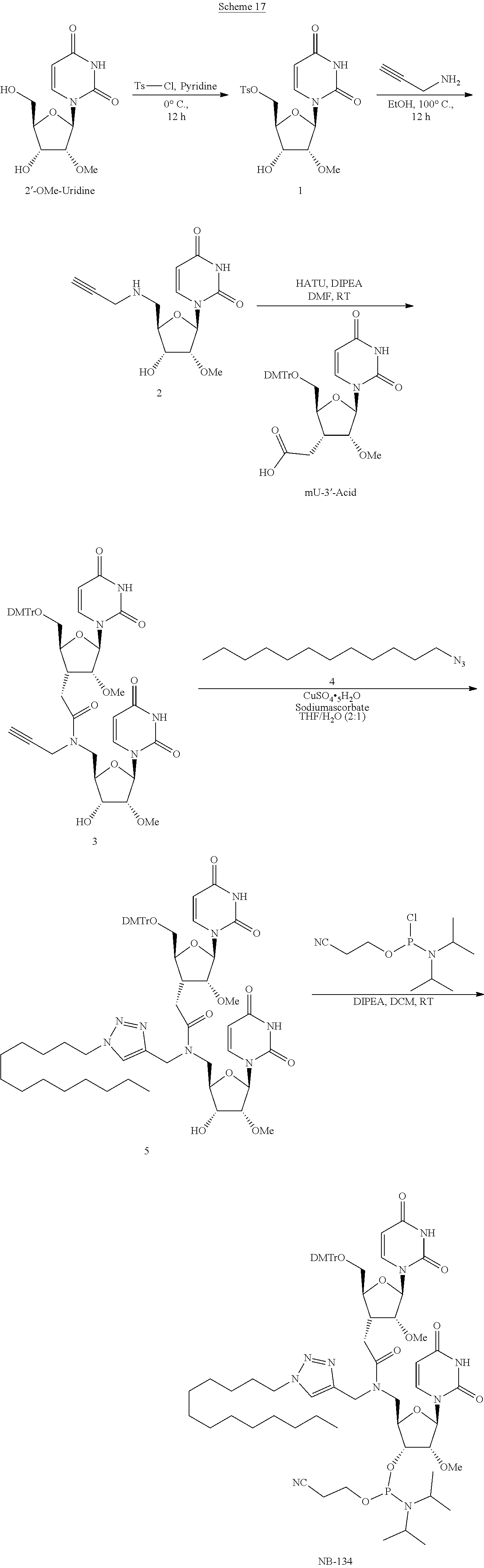

To a solution of diol (5 g, 19.380 mmol, 1 eq) in Pyridine 30 ml. was added p-tolylsulfonyl chloride (4 g, 21.318 mmol, 1.1 eq) in 20 ml. of dry pyridine dropwise. The reaction mixture was stirred at RT for 12 hr, LCMS showed mono and di-Tosyl. Reaction mixture was diluted with saturated aqueous sodium bicarbonate, 100 ml. Extracted with Ethyl acetate 200 mL, dried and concentrated, the crude residue was purified by column chromatography using 0-100% ethyl acetate/hexane as an eluent to obtain mixture of mono tosyl along with some di-tosyl (3.7 g 46%). LCMS (m/z=413 M+1) used as it is for next step.

A mixture of Tosyl 1 (3.7 g, 8.98 mmol, 1 eq) and Propargyl amine in EtOH (30 ml) was stirred for 12 h at 100° C. Reaction mixture was concentrated, the crude residue was purified by column chromatography using 0-10% MeOH/EtOAc as an eluent to obtain amine 2 (900 mg 30%) LCMS (m/z=296 M+1) as a beige solid.

A solution of acid mU-3′-Acid (3.1 g, 5.153 mmol, 1 eq), DIPEA (2.68 mL, 15.45 mmol, 3 eq), HATU (2.93 g, 7.729 mmol, 1.5 eq) and amine 2 (1.52 g, 5.153 mmol, 1.0 eq) in DMF (20 mL) was stirred for 3 h at room temperature, reaction mixture was diluted with water (50 ml), precipitated solids were filtered and washed with water, the solids were dried and re dissolved in DCM then purified by Ethyl acetate/MeOH, 0-10% as an eluent, pure fractions were combined and concentrated to obtain amide 3 (1.5 g, 33%) as a white solid. LCMS m/z 880 (M+1).

To a solution of alkyne 3 (1.4 g, 1.56 mmol, 1 eq) and azide 4 (495 mg, 2.07 mmol, 1.3 eq) in THF (15 mL) at RT was added a solution of CuSO₄·5H₂O (200 mg, 0.8 mmol, 0.5 eq) in water (2 mL) followed by a solution of sodium ascorbate (237 mg, 1.19 mmol, 0.75 eq) in water (2 mL). The reaction was stirred for 2 hrs at RT. Then the reaction was diluted with EtOAc 200 mL and washed with NaHCO₃ (100 mL), dried over Na₂SO₄, filtered, and concentrated. The residue was purified by column chromatography using 0-100% EtOAc/Hexane to give 1.43 g of triazole 5 (81%) as a beige solid, mass m/z=1106 (M+1).

To a stirred solution of alcohol 5 (1.4 g, 1.285 mmol, 1 eq) and diisopropylethylamine (1.43 mL, 7.71 mmol, 6 eq), in DCM (10 mL), was added N, N-diisopropyl chlorophosphoramidite (0.86 mL 3.86 mmol, 3 eq) dropwise. The reaction mixture was stirred at room temperature for 2 h. Reaction mixture was quenched with Aq. Saturated NaHCO³ solution (30 mL), extracted with DCM (100) ml, washed with brine (50 mL), dried over Na₂SO₄, and evaporated the crude product was loaded on to (pre-equilibrated with 1% Et₃N-DCM) Biotage silica gel column (100 g 20 μm), and purified by flash chromatography using MeOH/EtOAc 0-10% 5 CV then 10-10% 10 CV containing 1% Et₃N as an additive. Pure fractions were combined and concentrated, dried under high vacuum to obtain Phosphoramidite NB-134 (1.35 g, 80%) as a white solid. 95% purity by HPLC, mass (m/z 1327 M+Na), ³¹P NMR (202, MHz, DMSO-d6) δ 149.87, 149.69, 149.58.

Oligonucleotide Comprising Dinucleotide NB-134 has been Synthesized Using the General Procedure Described in Example 1.

### Example 25: NB-135

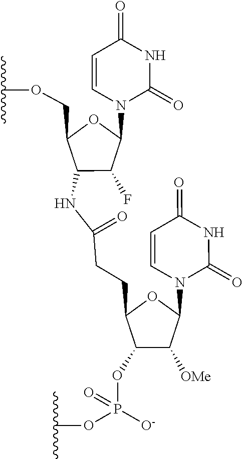

### Synthesis of NB-135

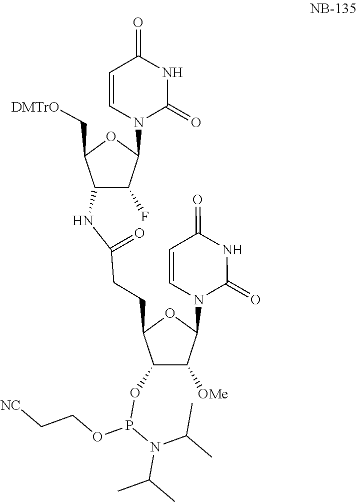

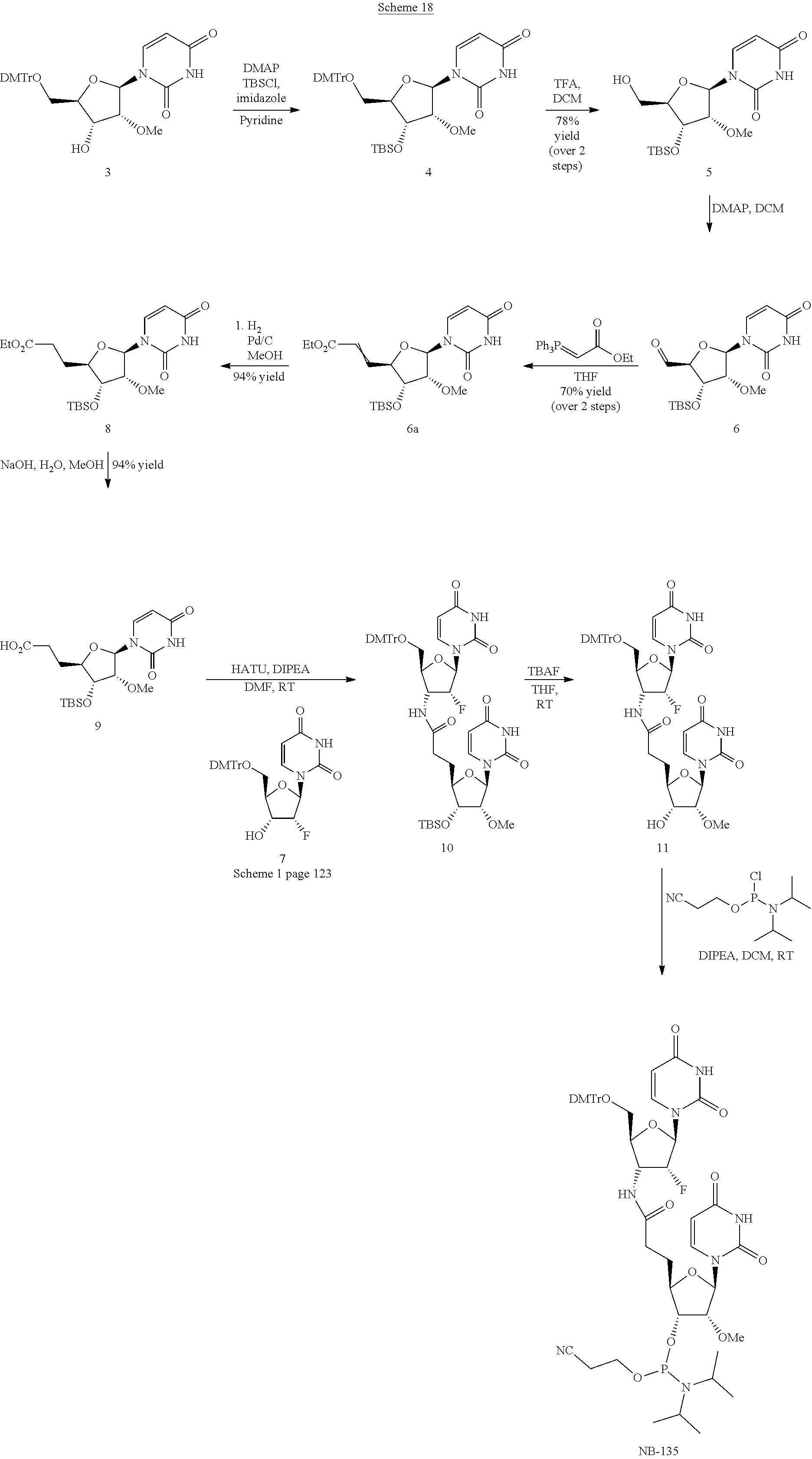

Imidazole (6.06 g, 89 mmol, 2.5 equiv) and TBSCl (8.07 g, 54 mmol, 1.5 equiv) were added as solids consecutively to a solution of alcohol 3 (20.0 g, 35.7 mmol, 1 equiv) in anhydrous pyridine (200 mL) at 0° C. After 21 h, 14% SM remains, add additional TBSCl (1.77 g, 11.8 mmol, 0.33 equiv) at 0° C., then stir at room temperature. After 4 h, the reaction mixture was concentrated under reduced pressure at 40° C. The resulting syrup was poured into stirring water (200 mL) and then extracted with ethyl acetate (150 mL). The aqueous layer was extracted with ethyl acetate (2×100 mL). The combined organic extracts were washed with sat'd NaCl (100 mL), dried over anhydrous sodium sulfate, filtered, and concentrated under reduced pressure to afford crude TBS ether 4 (24 g) that was used without further purification.

TFA (10 mL, 124 mmol, 7 equiv) was added dropwise to a solution of crude DMT ether 4 (12 g, 17.9 mmol, 1 equiv) in DCM (100 mL) at 0° C. The reaction mixture was then allowed to stir at room temperature. After 1 h, the reaction mixture was poured into stirring sat'd NaHCO₃ (300 mL). The aqueous layer was extracted with DCM (2×100 mL). The combined organic extracts were dried over anhydrous sodium sulfate, filtered, and concentrated under reduced pressure. The resulting residue was purified by FCC on silica gel (0□60% ethyl acetate in hexanes) to afford alcohol 5 as a white solid (5.33 g). This procedure was repeated on the remaining 12 g of crude DMT ether 4, which after combination afforded alcohol 5 (10.4 g, 78% yield over two steps).

Sonicate to dissolve alcohol 5 (1.5 g, 4.03 mmol, 1 equiv) in anhydrous DCM (40 mL). Then, Dess-Martin periodinane (2.14 g, 5.03 mmol, 1.25 equiv) was added as a solid in one portion, resulting in a pink/salmon colored suspension. After 3.5 h, the reaction mixture was poured into stirring sat'd sodium thiosulfate (150 mL) and extracted with DCM (60 mL). The organic layer was washed with sat'd sodium carbonate (150 mL) and with sat'd NaCl (150 mL). The aqueous layers were separately extracted with DCM (2×100 mL). The combined organic extracts were dried over anhydrous sodium sulfate, filtered, and concentrated under reduced pressure to afford crude aldehyde 6a (1.57 g) as a white solid, which was used without further purification.

Triphenylcarbethoxymethylenephosphorane (1.76 g, 5.06 mmol, 1.25 equiv) was added as a solid in one portion to a suspension of crude aldehyde 6a (1.5 g, 4.05 mmol, 1 equiv) in anhydrous THF (40 mL) at room temperature. After 16 h, the reaction mixture was concentrated under reduced pressure at 30° C. to remove THF. The resulting residue was extracted with ethyl acetate (100 mL) and washed with water (50 mL) and with sat'd NaCl (50 mL). The aqueous layer was extracted with EtOAc (50 mL). The combined organic extracts were dried over anhydrous sodium sulfate, filtered, and concentrated under reduced pressure. The resulting residue was purified by FCC on silica gel (0□50% ethyl acetate/hexanes) to afford ester 7 (1.24 g, 70% yield over 2 steps, 20:1 d.r. by UV).

A solution of alkene 7 (1.24 g, 2.81 mmol, 1 equiv) in methanol (20 mL) was evacuated and backfilled with nitrogen, and charged with palladium/carbon (300 mg, 0.28 mmol, 0.1 equiv). The reaction mixture was then evacuated and backfilled with hydrogen gas from a balloon. After 75 min, the reaction mixture was filtered through Celite and rinsed with methanol. The filtrate was concentrated under reduced pressure to afford ester 8 (1.17 g, 94% yield) as a white foam, which was used without further purification.

A solution of sodium hydroxide (528 mg, 13.2 mmol, 5 equiv) in water (1 mL) was added dropwise to a solution of crude ester 8 (1.17 g, 2.64 mmol, 1 equiv) in methanol (10 mL) at room temperature. After 1.5 h, the reaction mixture was concentrated under reduced pressure. The resulting residue was diluted with water (˜20 mL) resulting in a white precipitate. The reaction mixture was cooled to 0° C. with stirring, then acidified by the dropwise addition of 1 N HCl (14 mL), resulting in pH=3-4. The reaction mixture was then extracted with DCM (3×50 mL). The combined organic extracts were dried over anhydrous sodium sulfate, filtered, and concentrated under reduced pressure to afford carboxylic acid 9 (1.03 g, 94% yield) as a white foam.

A solution of acid 9 (0.611 g, 1.476 mmol, 1 eq), DIPEA (0.77 mL, 4.427 mmol, 3 eq), HATU (0.841 g, 2.214 mmol, 1.5 eq) and mixture of amine 7 (1.16 g, 1.476 mmol, 1.0 eq) in DMF (10 mL) was stirred for 3 h at room temperature, reaction mixture was diluted with water (50 ml), precipitated solids were filtered and washed with water, the solids were dried and re dissolved in DCM then purified by Ethyl acetate/MeOH, 0-10% as an eluent, pure fractions were combined and concentrated to obtain amide 10 (0.9 g, 64%) as a white solid, LCMS m/z 945 (M+1).

To a stirred solution of TBDMS ether 10 (0.800 g, 0.847 mmol, 1 eq) in anhydrous THF (10 mL) was added TBAF (0.8 mL, 2.542 mmol, 3 eq), the mixture was stirred at room temperature for 12 h hours. LCMS showed complete deprotection, the reaction was diluted with EtOAc (200 mL) and the organic phase was sequentially washed with, saturated NaHC0₃, brine, dried (Na₂SO₄) and concentrated under vacuum. The residue was purified by column chromatography using 0-10% MeOH/DCM as eluent pure fractions were combined and concentrated to obtain alcohol 11 (650 mg 92%) as a beige solid, (M/z 852 M+Na).

To a stirred solution of alcohol 11 (0.677 g, 0.817 mmol, 1 eq) and diisopropylethylamine (0.85 mL, 4.9 mmol, 6 eq), in DCM (10 mL), was added N, N-diisopropyl chlorophosphoramidite (0.54 mL 2.45 mmol, 3 eq) dropwise. The reaction mixture was stirred at room temperature for 2 h. Reaction mixture was quenched with Aq. Saturated NaHCO₃ solution (30 mL), extracted with DCM (50) ml, washed with brine (50 mL), dried over Na₂SO₄, and evaporated the crude product was loaded on to (pre-equilibrated with 1% Et₃N-DCM) Biotage silica gel column (50 g 20 μm), and purified by flash chromatography using MeOH/EtOAc 0-10% 5 CV then 10-10% 10 CV containing 1% Et₃N as an additive. Pure fractions were combined and concentrated, dried under high vacuum to obtain Phosphoramidite NB-135 (540 mg, 64%) as a white solid. 95% purity by HPLC, Mass (m/z 1052 M+Na), ³¹P NMR (202, MHz, DMSO-d6) δ 149.56.

Oligonucleotide Comprising Dinucleotide NB-135 has been Synthesized Using the General Procedure Described in Example 1.

### Example 26: NB-136

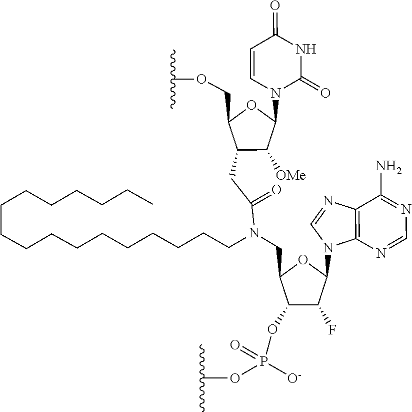 [SMILES: *OC[C@H]1O[C@@H](n2ccc(=O)[nH]c2=O)[C@H](OC)[C@@H]1CC(=O)N(CCCCCCCCCCCCCCCCC)C[C@H]1O[C@@H](n2cnc3c(N)ncnc32)[C@H](F)[C@@H]1OP(=O)([O-])O*]

### Synthesis of NB-136: (2R,3R,4R,5R)-5-(6-benzamido-9H-purin-9-yl)-2-((2-((2S,3R,4R,5R)-2-((bis(4-methoxyphenyl)(phenyl)methoxy)methyl)-5-(2,4-dioxo-3,4-dihydropyrimidin-1(2H)-yl)-4-methoxytetrahydrofuran-3-yl)-N-heptadecylacetamido)methyl)-4-fluoro tetrahydrofuran-3-yl(2-cyanoethyl) diisopropylphosphoramidite

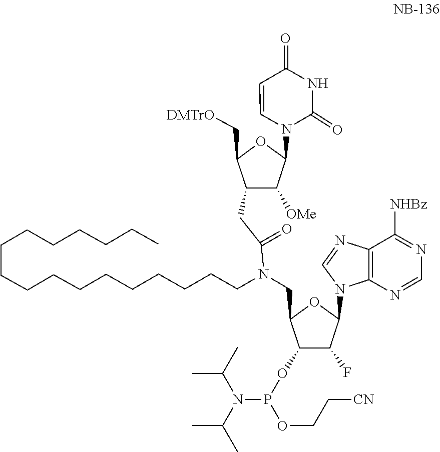

Dinucleotide NB-136 has been synthesized following the procedure described for compound NB-130 (example 20). Mass, 1395 [M+H]⁺.

Oligonucleotide Comprising Dinucleotide NB-136 has been Synthesized Using the General Procedure Described in Example 1.

### Example 27: NB-137

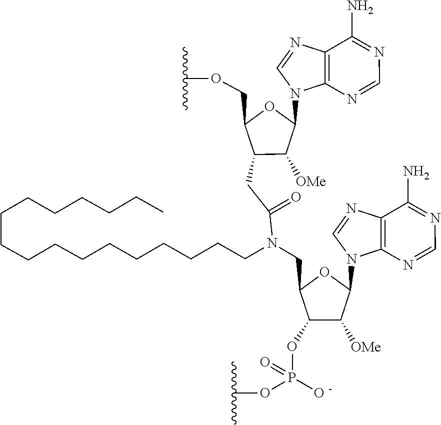 [SMILES: *OC[C@H]1O[C@@H](n2cnc3c(N)ncnc32)[C@H](OC)[C@@H]1CC(=O)N(CCCCCCCCCCCCCCCCC)C[C@H]1O[C@@H](n2cnc3c(N)ncnc32)[C@H](OC)[C@@H]1OP(=O)(O)O*]

### Synthesis of NB-137

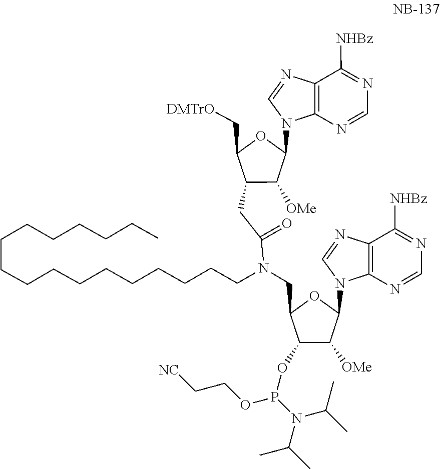

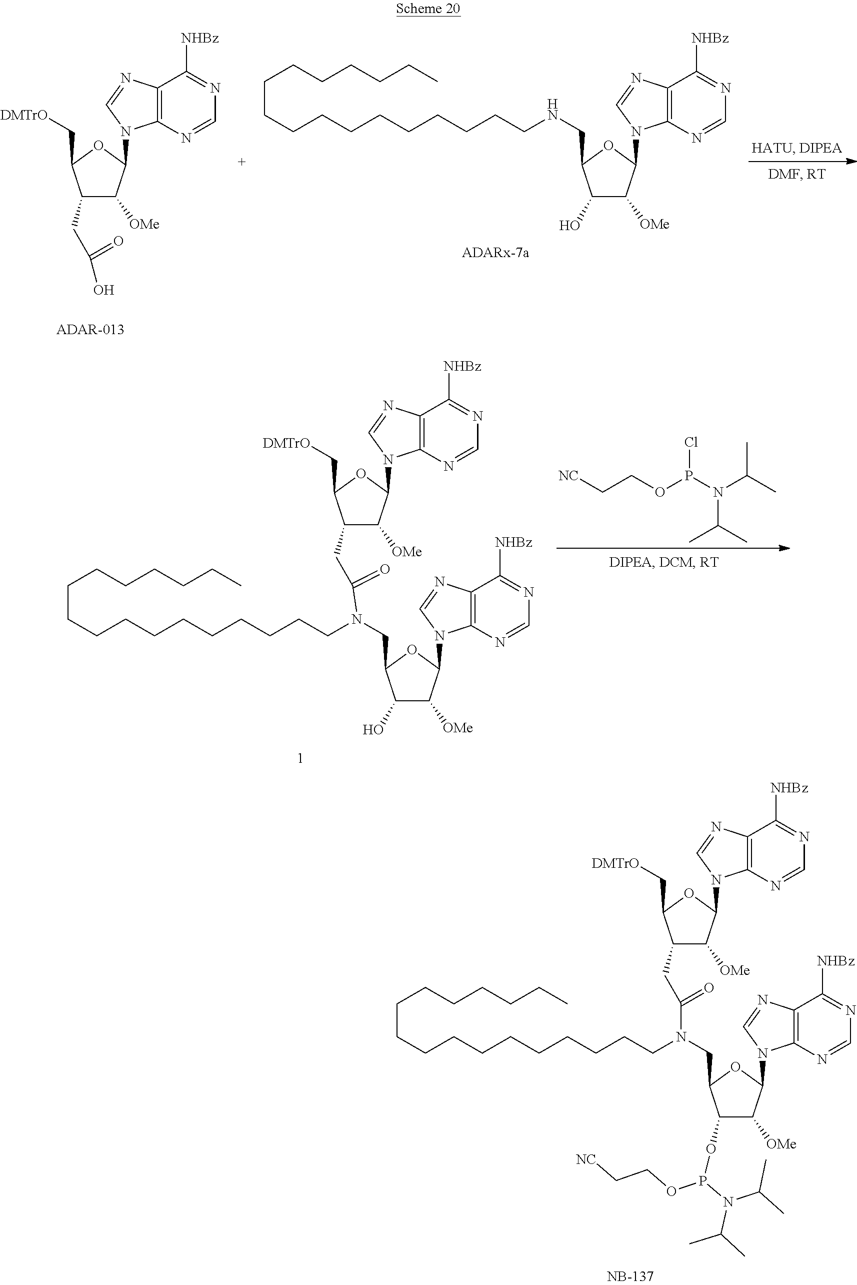

To a stirred solution of acid ADAR-013 (0.604 g, 0.829 mmol, 1 eq), amine ADARx-7a (0.515 g, 0.829 mmol, 1 eq) and HATU (0.472 g, 1.24 mmol, 1.5 eq) in DMF (10 mL) was added DIPEA (0.43 mL, 2.48 mmol, 3 eq) and the mixture was stirred for 3 h at room temperature, reaction mixture was diluted with water (50 ml), precipitated solids were filtered and washed with water, the solids were dried and re dissolved in DCM then purified by Ethyl acetate/MeOH, 0-5% as an eluent, pure fractions were combined and concentrated to obtain amide 1 (1 g, 91%) as a beige solid. LCMS m/z 1356 (M+Na).

To a stirred solution of alcohol 1 (1 g, 0.753 mmol, 1 eq) and diisopropylethylamine (0.785 mL, 4.5 mmol, 6 eq), in DCM (10 mL), was added N, N-diisopropyl chlorophosphoramidite (0.5 mL 2.25 mmol, 3 eq) dropwise. The reaction mixture was stirred at room temperature for 2 h. Reaction mixture was quenched with Aq. Saturated NaHCO₃ solution (30 mL), extracted with DCM (50) ml, washed with brine (50 mL), dried over Na₂SO₄, and evaporated the crude product was loaded on to (pre-equilibrated with 1% Et₃N-DCM) Biotage silica gel column (100 g 20 μm), and purified by flash chromatography using MeOH/EtOAc 0-10% 5 CV then 10-10% 10 CV containing 1% Et₃N as an additive. Pure fractions were combined and concentrated, dried under high vacuum to obtain Phosphoramidite NB-137 (600 mg, 52%) product as a white solid. >95% purity by HPLC, Mass (m/z 1556 M+Na), ³¹P NMR (202, MHz, DMSO-d6) δ 150.22, 149.78, 149.63.

Oligonucleotide Comprising Dinucleotide NB-137 has been Synthesized Using the General Procedure Described in Example 1.

### Example 28: NB-138

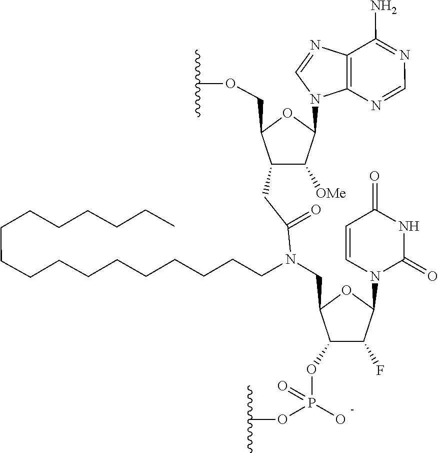 [SMILES: *OC[C@H]1O[C@@H](n2cnc3c(N)ncnc32)[C@H](OC)[C@@H]1CC(=O)N(CCCCCCCCCCCCCCCCC)C[C@H]1O[C@@H](n2ccc(=O)[nH]c2=O)[C@H](F)[C@@H]1OP(=O)(O)O*]

### Synthesis of NB-138

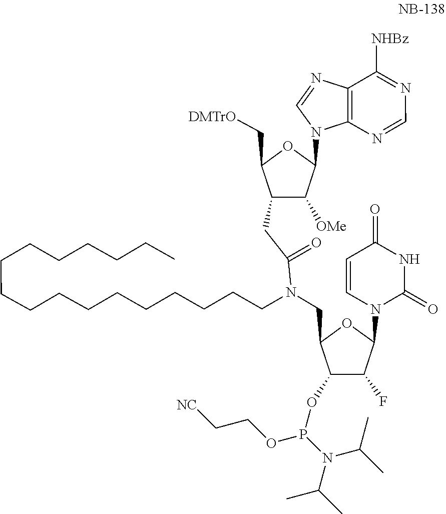

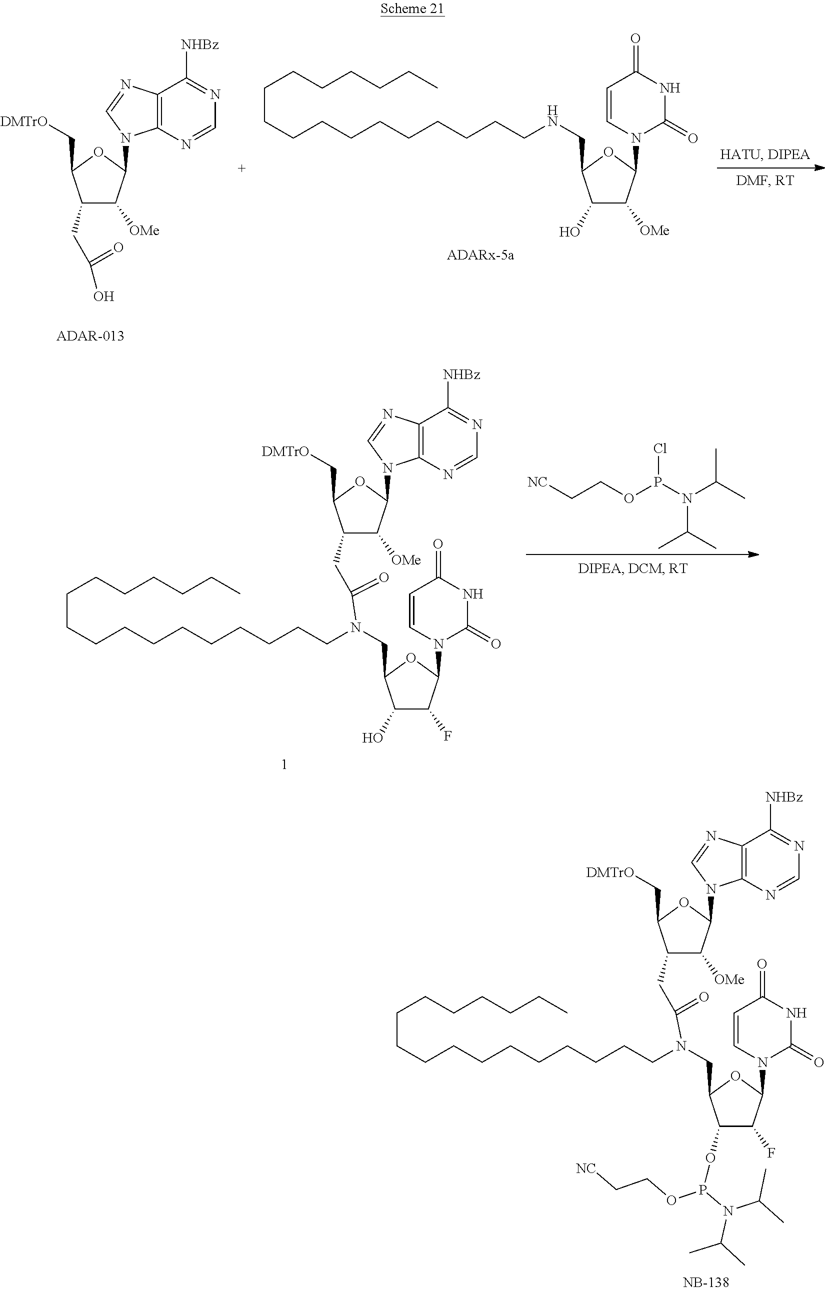

To a stirred solution of acid ADAR-013 (1 g, 1.37 mmol, 1 eq), amine ADARx-5a (0.729 g, 1.51 mmol, 1.1 eq) and HATU (0.782 g, 2.06 mmol, 1.5 eq) in DMF (20 mL) was added DIPEA (0.715 mL, 4.11 mmol, 3 eq) and the mixture was stirred for 3 h at room temperature, reaction mixture was diluted with water (50 ml), precipitated solids were filtered and washed with water, the solids were dried and re dissolved in DCM then purified by Ethyl acetate/MeOH, 0-5% as an eluent, pure fractions were combined and concentrated to obtain amide 1 (1.15 g, 64%) as a beige solid. LCMS m/z 1196 (M+1).

To a stirred solution of alcohol 1 (1.136 g, 0.951 mmol, 1 eq) and diisopropylethylamine (1 mL, 5.7 mmol, 6 eq), in DCM (10 mL), was added N, N-diisopropyl chlorophosphoramidite (0.63 mL 2.85 mmol, 3 eq) dropwise. The reaction mixture was stirred at room temperature for 2 h. Reaction mixture was quenched with Aq. Saturated NaHCO₃ solution (30 mL), extracted with DCM (50) ml, washed with brine (50 mL), dried over Na₂SO₄, and evaporated the crude product was loaded on to (pre-equilibrated with 1% Et₃N-DCM) Biotage silica gel column (100 g 20 μm), and purified by flash chromatography using MeOH/EtOAc 0-10% 5 CV then 10-10% 10 CV containing 1% Et₃N as an additive. Pure fractions were combined and concentrated, dried under high vacuum to obtain NB-138 (900 mg, 68%) as an off-white solid. 93% purity by HPLC, Mass (m/z 1418 M+Na), ³¹P NMR (202, MHz, DMSO-d6) δ 150.23, 149.72.

Oligonucleotide Comprising Dinucleotide NB-138 has been Synthesized Using the General Procedure Described in Example 1.

### Example 29: NB-139

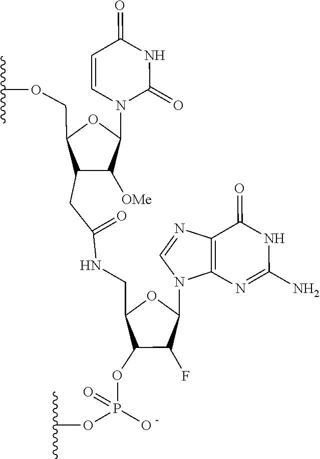

### Synthesis of NB-139

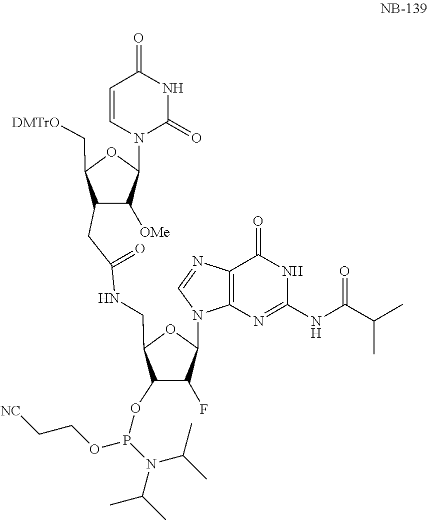

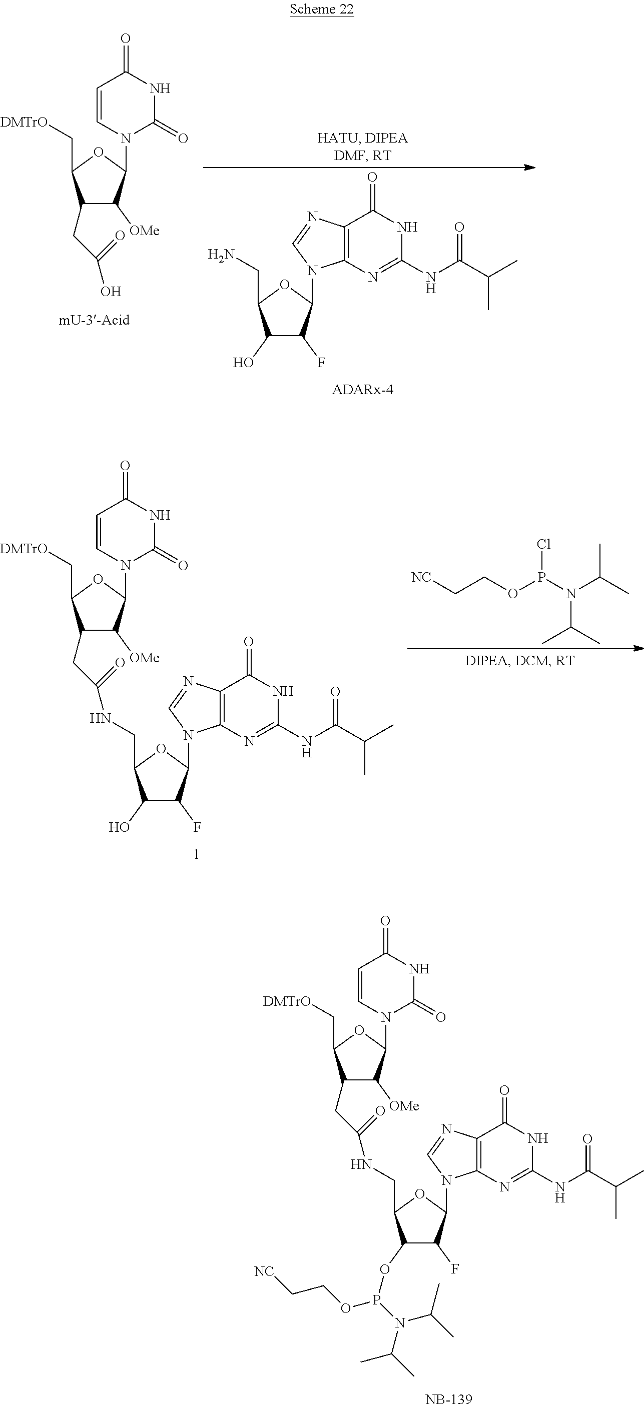

To a stirred solution of mU-3′-acid (1.5 g, 2.49 mmol, 1 eq), amine ADARx-4 (0.970 g, 2.74 mmol, 1.1 eq) and HATU (1.42 g, 3.738 mmol, 1.5 eq) in DMF (10 mL) was added DIPEA (1.3 mL, 7.47 mmol, 3 eq) and the mixture was stirred for 3 h at room temperature, reaction mixture was diluted with saturated NaHCO₃ (50 ml), precipitated solids were filtered and washed with water, the solids were dried and re dissolved in DCM then purified by DCM/MeOH, 0-10% as an eluent, pure fractions were combined and concentrated to obtain amide 1 (1.61 g, 63%) as a beige solid. LCMS m/z 961 (M+Na).

To a stirred solution of alcohol 1 (1.6 g, 1.716 mmol, 1 eq) and diisopropylethylamine (1.8 mL, 10.3 mmol, 6 eq), in DCM (20 mL), was added N, N-diisopropyl chlorophosphoramidite (1.15 mL 5.15 mmol, 3 eq) dropwise. The reaction mixture was stirred at room temperature for 2 h. Reaction mixture was quenched with Aq. Saturated NaHCO₃ solution (10 mL), extracted with DCM (100) ml, washed with brine (50 mL), dried over Na₂SO₄, and evaporated the crude product was loaded on to (pre-equilibrated with 1% Et₃N-DCM) Biotage silica gel column (100 g 20 μm), and purified by flash chromatography using MeOH/DCMO-5% 5 CV then 10-10% 10 CV containing 1% Et₃N as an additive. Pure fractions were combined and concentrated, dried under high vacuum to obtain Phosphoramidite NB-139 (1.67 g, 85%) as a white solid. 95% purity by HPLC, Mass (m/z 1137 M+−1). ³¹P NMR (202, MHz, DMSO-d6) δ 150.76, 150.70, 150.36, 150.33.

Oligonucleotide Comprising Dinucleotide NB-139 has been Synthesized Using the General Procedure Described in Example 1.

### Example 30: NB-140

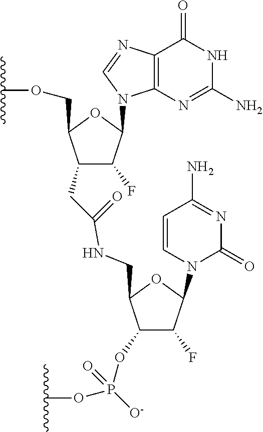 [SMILES: *OC[C@H]1O[C@@H](n2cnc3c(=O)[nH]c(N)nc32)[C@H](F)[C@@H]1CC(=O)NC[C@H]1O[C@@H](n2ccc(N)nc2=O)[C@H](F)[C@@H]1OP(=O)([O-])O*]

### Synthesis of NB-140

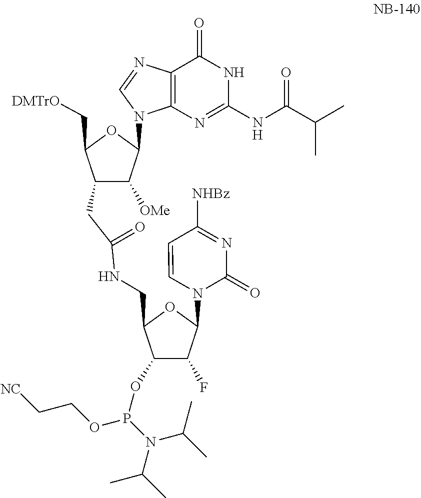

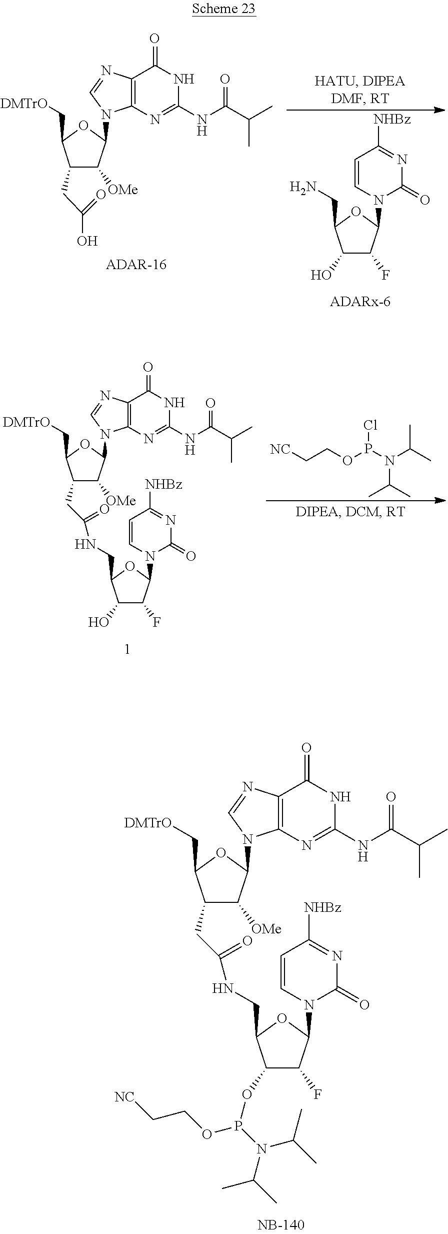

To a stirred solution of acid ADAR-16 (1.5 g, 2.11 mmol, 1 eq), amine ADARx-6 (0.947 g, 2.32 mmol, 1.1 eq) and HATU (1.2 g, 3.16 mmol, 1.5 eq) in DMF (10 mL) was added DIPEA (1.1 mL, 6.33 mmol, 3 eq) and the mixture was stirred for 3 h at room temperature, reaction mixture was diluted with saturated NaHCO₃ (50 ml), precipitated solids were filtered and washed with water, the solids were dried and re dissolved in DCM then purified by DCM/MeOH, 0-10% as an eluent, pure fractions were combined and concentrated to obtain amide 1 (0.7 g, 29%) as a beige solid. LCMS m/z 1042 (M+).

To a stirred solution of alcohol 1 (0.667 g, 0.64 mmol, 1 eq) and diisopropylethylamine (0.67 mL, 3.84 mmol, 6 eq), in DCM (10 mL), was added N, N-diisopropyl chlorophosphoramidite (0.42 mL 1.92 mmol, 3 eq) dropwise. The reaction mixture was stirred at room temperature for 2 h. Reaction mixture was quenched with Aq. Saturated NaHCO₃ solution (30 mL), extracted with DCM (50) ml, washed with brine (50 mL), dried over Na₂SO₄, and evaporated the crude product was loaded on to (pre-equilibrated two times with 1% Et₃N-DCM) Biotage silica gel column (50 g 20 μm), and purified by flash chromatography using MeOH/EtOAc 0-10% 5 CV then 10-10% 10 CV containing 1% Et₃N as an additive. Pure fractions were combined and concentrated, dried under high vacuum to obtain Phosphoramidite NB-140 (0.66 mg, 83%) as a white solid. 92% purity by HPLC, Mass (m/z 1264 M+Na), ³¹P NMR (202, MHz, DMSO-d6) δ 150.61, 150.52.

Oligonucleotide Comprising Dinucleotide NB-140 has been Synthesized Using the General Procedure Described in Example 1.

### Example 31: NB-141

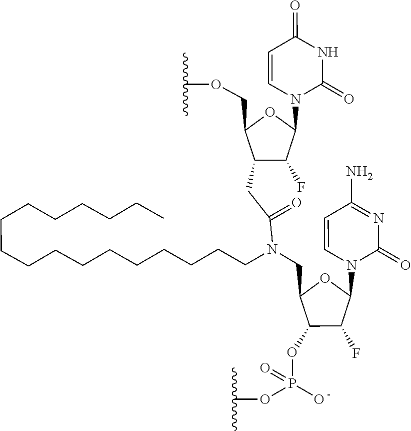 [SMILES: *OC[C@H]1O[C@@H](n2ccc(=O)[nH]c2=O)[C@H](F)[C@@H]1CC(=O)N(CCCCCCCCCCCCCCCCC)C[C@H]1O[C@@H](n2ccc(N)nc2=O)[C@H](F)[C@@H]1OP(=O)(O)O*]

### Synthesis of NB-141

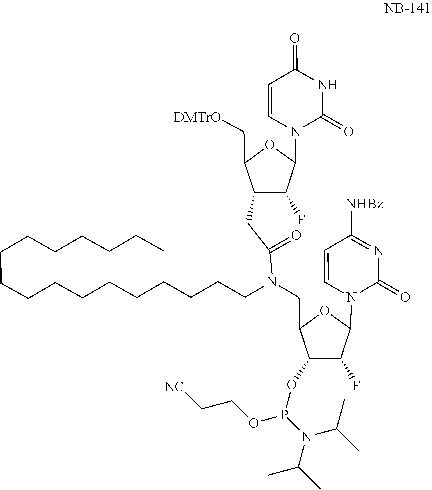

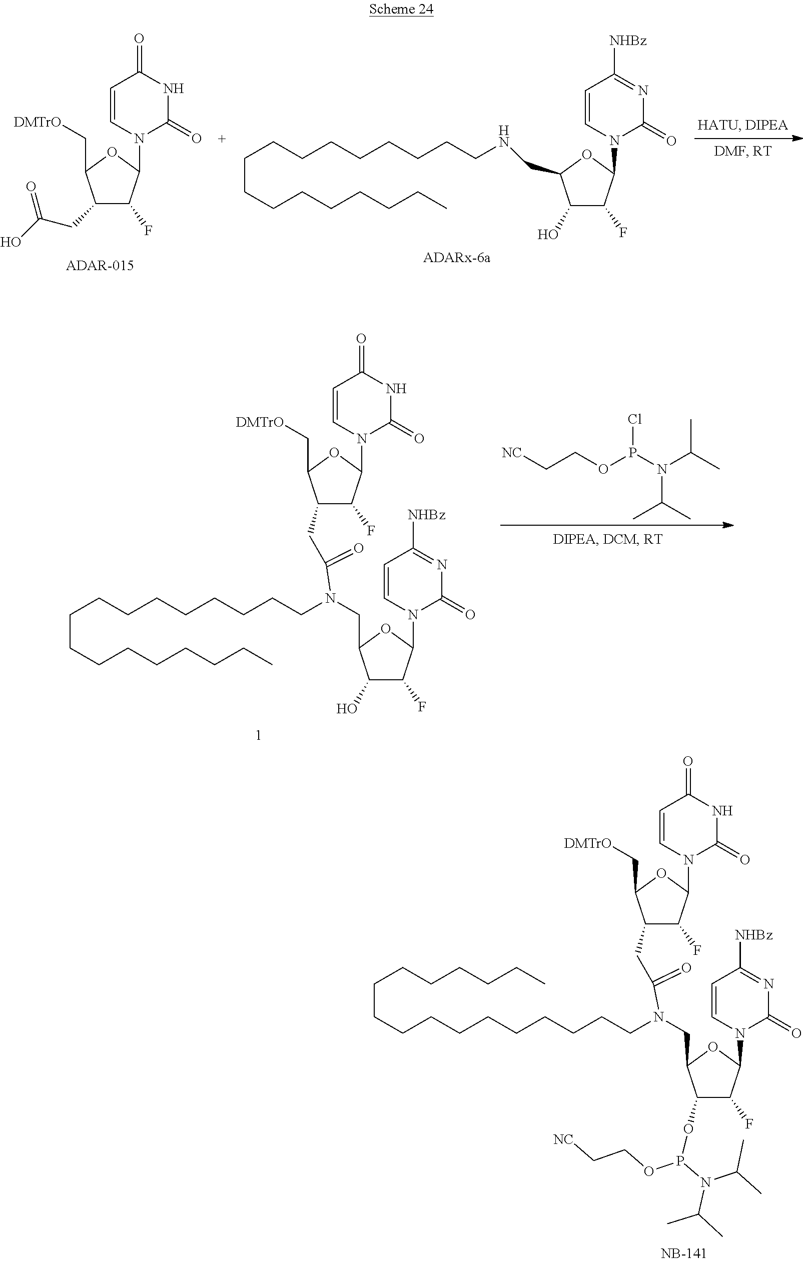

A solution of acid ADAR-15 (1.45 g, 2.46 mmol, 1 eq), DIPEA (1.28 mL, 7.4 mmol, 3 eq), HATU (1.4 g, 3.7 mmol, 1.5 eq) and amine ADARx-6a (1.44 g, 2.4 mmol, 1.0 eq) in DMF (10 mL) was stirred for 3 h at room temperature, reaction mixture was diluted aq. Saturated sodium bicarbonate (50 mL), extracted with DCM (2×100 mL), dried over Na₂SO₄ and concentrated the crude product was purified by Ethyl acetate/MeOH, 0-10% as an eluent, pure fractions were combined and concentrated to obtain amide 1 (1.65 g, 57.6%) as a beige solid. Mass m/z 1181 (M+Na).

To a stirred solution of alcohol 1 (1.63 g, 1.406 mmol, 1 eq) and diisopropylethylamine (1.46 mL, 8.43 mmol, 6 eq), in DCM (20 mL), was added N, N-diisopropyl chlorophosphoramidite (0.93 mL 4.22 mmol, 3 eq) dropwise. The reaction mixture was stirred at room temperature for 2 h. Reaction mixture was quenched with Aq. Saturated NaHCO₃ solution (10 mL), extracted with DCM (100) ml, washed with brine (50 mL), dried over Na₂SO₄, and evaporated the crude product was loaded on to (pre-equilibrated with 1% Et₃N-DCM) Biotage silica gel column (100 g 20 μm), and purified by flash chromatography using MeOH/DCM0-5% 5 CV then 10-10% 10 CV containing 1% Et₃N as an additive. Pure fractions were combined and concentrated, dried under high vacuum to obtain Phosphoramidite NB-141 (1.48 g, 77%) as a beige solid. 93% purity by HPLC, Mass (m/z 1359 M+), ³¹P NMR (202, MHz, DMSO-d6) δ 150.75, 150.31, 150.05.

Oligonucleotide Comprising Dinucleotide NB-141 has been Synthesized Using the General Procedure Described in Example 1.

### Example 32: NB-142

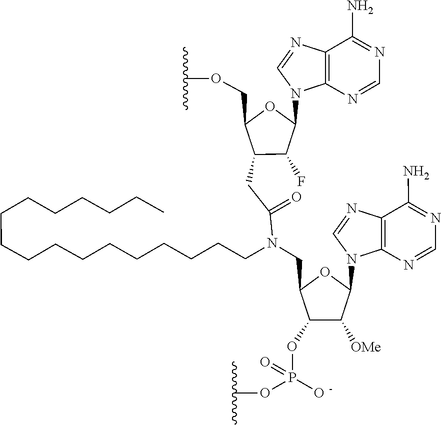 [SMILES: *OC[C@H]1O[C@@H](n2cnc3c(N)ncnc32)[C@H](F)[C@@H]1CC(=O)N(CCCCCCCCCCCCCCCCC)C[C@H]1O[C@@H](n2cnc3c(N)ncnc32)[C@H](OC)[C@@H]1OP(=O)(O)O*]

### Synthesis of NB-142

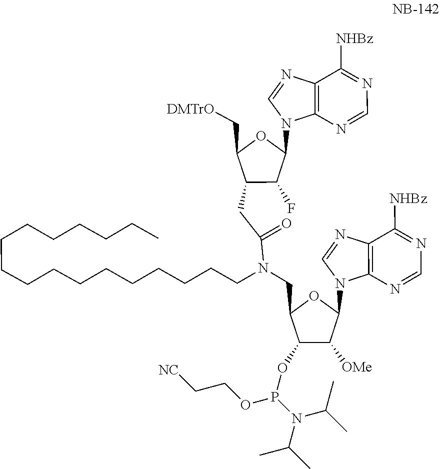

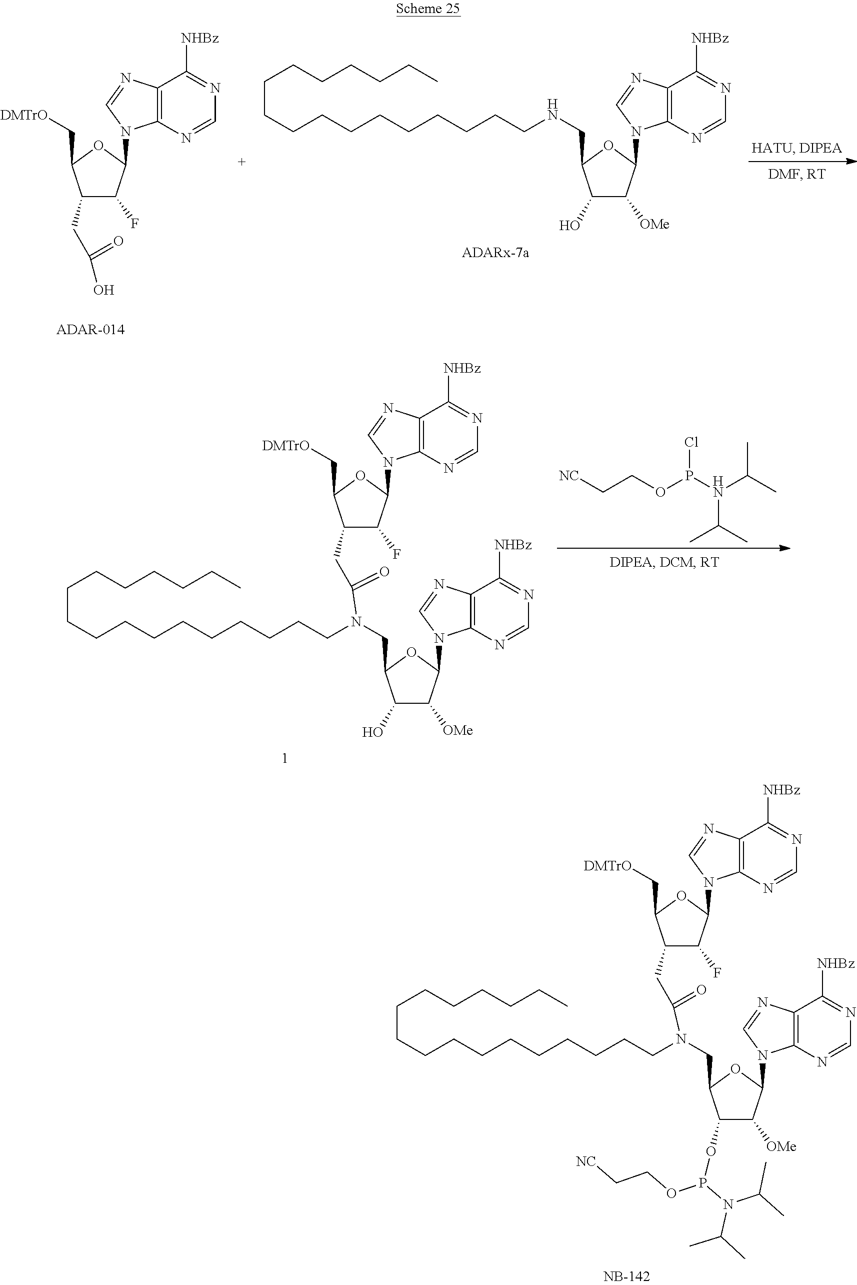

To a stirred solution of acid ADAR-14 (0.900 g, 1.255 mmol, 1 eq), amine ADARx-7a (0.781 g, 1.255 mmol, 1 eq) and HATU (0.715 g, 1.88 mmol, 1.5 eq) in DMF (10 mL) was added DIPEA (0.65 mL, 3.76 mmol, 3 eq) and the mixture was stirred for 3 h at room temperature, reaction mixture was added dropwise to a vigorously stirring water (50 ml), precipitated solids were filtered and washed with water, the solids were dried and re dissolved in DCM then purified by Ethyl acetate/MeOH, 0-5% as an eluent, pure fractions were combined and concentrated to obtain amide 1 (0.87 g, 52%) as a beige solid. LCMS m/z 1344 (M+Na).

To a stirred solution of alcohol 1 (0.86 g, 0.65 mmol, 1 eq) and diisopropylethylamine (0.68 mL, 3.9 mmol, 6 eq), in DCM (20 mL), was added N, N-diisopropyl chlorophosphoramidite (0.43 mL 1.95 mmol, 3 eq) dropwise. The reaction mixture was stirred at room temperature for 2 h. Reaction mixture was quenched with Aq. Saturated NaHCO₃ solution (10 mL), extracted with DCM (100) ml, washed with brine (50 mL), dried over Na₂SO₄, and evaporated the crude product was loaded on to (pre-equilibrated with 1% Et₃N-DCM) Biotage silica gel column (100 g 20 μm), and purified by flash chromatography using MeOH/DCM 0-5% 5 CV then 10-10% 10 CV containing 1% Et₃N as an additive. Pure fractions were combined and concentrated, dried under high vacuum to obtain Phosphoramidite NB-142 (0.75 g, 76%) as a pale-yellow solid. 95% purity by HPLC, Mass (m/z 1544 M+Na). ³¹P NMR (202, MHz, DMSO-d6) δ 150.22, 149.74, 149.59.

Oligonucleotide Comprising Dinucleotide NB-142 has been Synthesized Using the General Procedure Described in Example 1.

### Example 33: NB-143

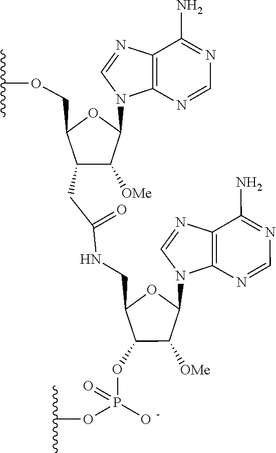 [SMILES: *OC[C@H]1O[C@@H](n2cnc3c(N)ncnc32)[C@H](OC)[C@@H]1CC(=O)NC[C@H]1O[C@@H](n2cnc3c(N)ncnc32)[C@H](OC)[C@@H]1OP(=O)(O)O*]

### Synthesis of NB-143

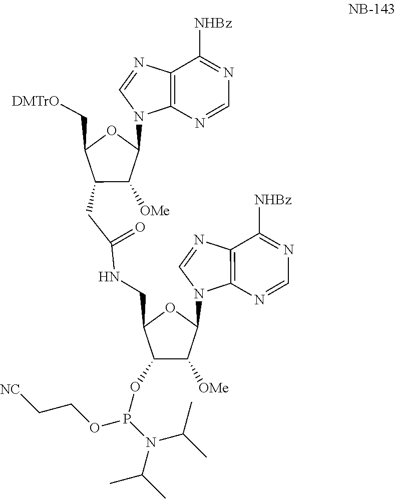

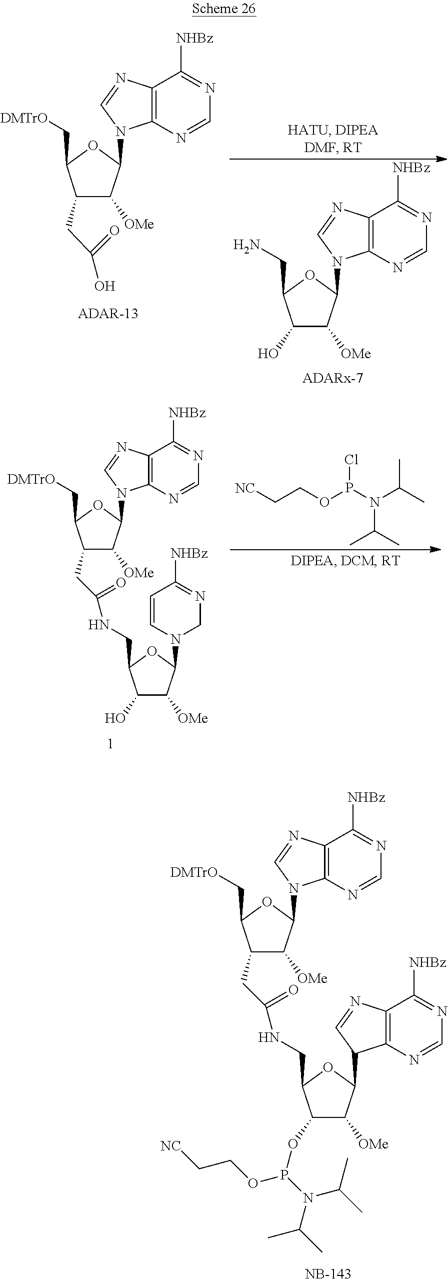

To a stirred solution of acid ADAR-13 (1.3 g, 1.78 mmol, 1 eq), amine ADARx-7 (0.685 g, 1.78 mmol, 1 eq) and HATU (1 g, 2.6 mmol, 1.5 eq) in DMF (10 mL) was added DIPEA (0.93 mL, 5.35 mmol, 3 eq) and the mixture was stirred for 3 h at room temperature, reaction mixture was added dropwise to a vigorously stirring water (50 ml), precipitated solids were filtered and washed with water, the solids were dried and re dissolved in DCM then purified by Ethyl acetate/MeOH, 0-5% as an eluent, pure fractions were combined and concentrated to obtain amide 1 (1.04 g, 53%) as a beige solid. LCMS m/z 1097 (M+1).

To a stirred solution of alcohol 1 (1.03 g, 0.94 mmol, 1 eq) and diisopropylethylamine (0.98 mL, 5.64 mmol, 6 eq), in DCM (20 mL), was added N, N-diisopropyl chlorophosphoramidite (0.62 mL 2.8 mmol, 3 eq) dropwise. The reaction mixture was stirred at room temperature for 2 h. Reaction mixture was quenched with Aq. Saturated NaHCO₃ solution (10 mL), extracted with DCM (100) ml, washed with brine (50 mL), dried over Na₂SO₄, and evaporated the crude product was loaded on to (pre-equilibrated with 1% Et₃N-DCM) Biotage silica gel column (100 g 20 μm), and purified by flash chromatography using MeOH/DCM0-5% 5 CV then 10-10% 10 CV containing 1% Et₃N as an additive. Pure fractions were combined and concentrated, dried under high vacuum to obtain

Phosphoramidite NB-143 (0.91 g, 75%) as a beige solid. 95% purity by HPLC, Mass (m/z 1318 M+Na), ³¹P NMR (202, MHz, DMSO-d6) δ 150.33, 149.66.

Oligonucleotide Comprising Dinucleotide NB-143 has been Synthesized Using the General Procedure Described in Example 1.

### Example 34: NB-144

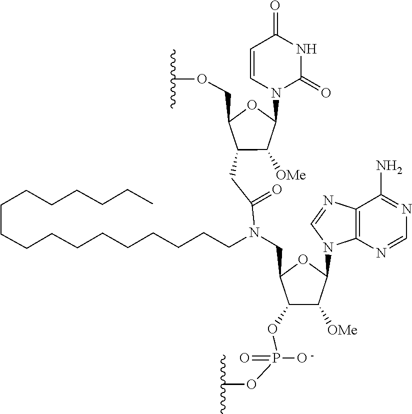 [SMILES: *OC[C@H]1O[C@@H](n2ccc(=O)[nH]c2=O)[C@H](OC)[C@@H]1CC(=O)N(CCCCCCCCCCCCCCCCC)C[C@H]1O[C@@H](n2cnc3c(N)ncnc32)[C@H](OC)[C@@H]1OP(=O)(O)O*]

### Synthesis of NB-144: (2R,3R,4R,5R)-5-(6-benzamido-9H-purin-9-yl)-2-((2-((2S,3R,4R,5R)-2-((bis(4-methoxyphenyl)(phenyl)methoxy)methyl)-5-(2,4-dioxo-3,4-dihydropyrimidin-1(2H)-yl)-4-methoxytetrahydrofuran-3-yl)-N-heptadecylacetamido)methyl)-4-methoxytetrahydrofuran-3-yl (2-cyanoethyl) diisopropylphosphoramidite

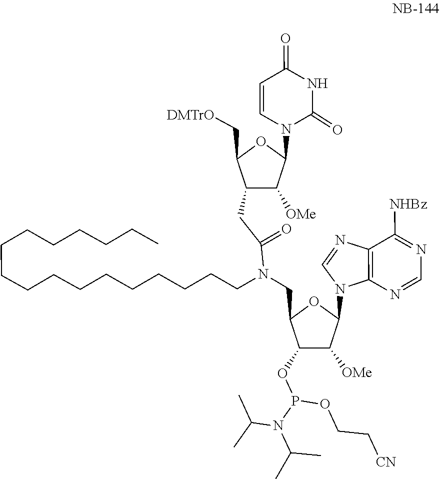

Dinucleotide NB-144 has been synthesized following the procedure described for compound NB-130 (example 20). Mass (m/z 1407, M+H), ³¹P NMR (202, MHz, DMSO-d6) δ 150.49, 150.42, 150.21, 150.03, 149.83, 147.74, 149.60.

Oligonucleotide Comprising Dinucleotide NB-144 has been Synthesized Using the General Procedure Described in Example 1.

### Example 35: NB-145

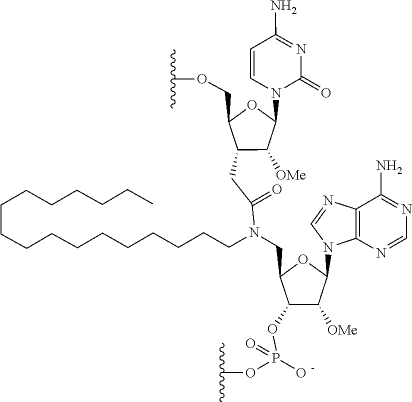 [SMILES: *OC[C@H]1O[C@@H](n2ccc(N)nc2=O)[C@H](OC)[C@@H]1CC(=O)N(CCCCCCCCCCCCCCCCC)C[C@H]1O[C@@H](n2cnc3c(N)ncnc32)[C@H](OC)[C@@H]1OP(=O)(O)O*]

### Synthesis of NB-145:(2R,3R,4R,5R)-2-((2-((2S,3R,4R,5R)-5-(4-benzamido-2-oxopyrimidin-1(2H)-yl)-2-((bis(4-methoxyphenyl)(phenyl)methoxy) methyl)-4-methoxytetrahydrofuran-3-yl)-N-heptadecylacetamido)methyl)-5-(6-benzamido-9H-purin-9-yl)-4-methoxytetrahydrofuran-3-yl(2-cyanoethyl) diisopropylphosphoramidite

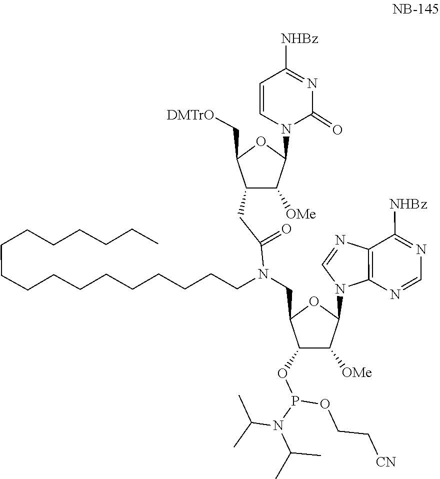

Dinucleotide NB-145 has been synthesized following the procedure described for compound NB-130 (example 20). Mass (m/z 1532, M+Na), ³¹P NMR (202, MHz, DMSO-d6) δ 150.14, 149.85, 149.78, 147.94.

Oligonucleotide Comprising Dinucleotide NB-145 has been Synthesized Using the General Procedure Described in Example 1.

### Example 36: NB-146

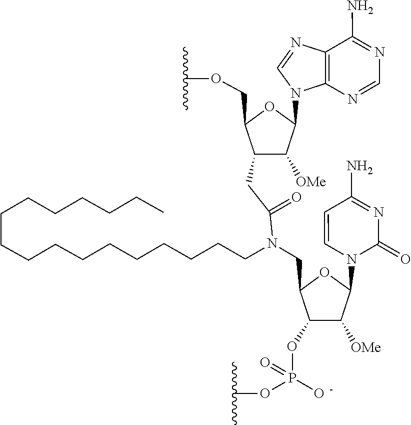 [SMILES: *OC[C@H]1O[C@@H](n2cnc3c(N)ncnc32)[C@H](OC)[C@@H]1CC(=O)N(CCCCCCCCCCCCCCCCC)C[C@H]1O[C@@H](n2ccc(N)nc2=O)[C@H](OC)[C@@H]1OP(=O)(O)O*]

### Synthesis of NB-146: (2R,3R,4R,5R)-5-(4-benzamido-2-oxopyrimidin-1(2H)-yl)-2-((2-((2S,3R,4R,5R)-5-(6-benzamido-9H-purin-9-yl)-2-((bis(4-methoxyphenyl)(phenyl)methoxy)methyl)-4-methoxytetrahydrofuran-3-yl)-N-heptadecylacetamido)methyl)-4-methoxytetrahydrofuran-3-yl (2-cyanoethyl) diisopropylphosphoramidite

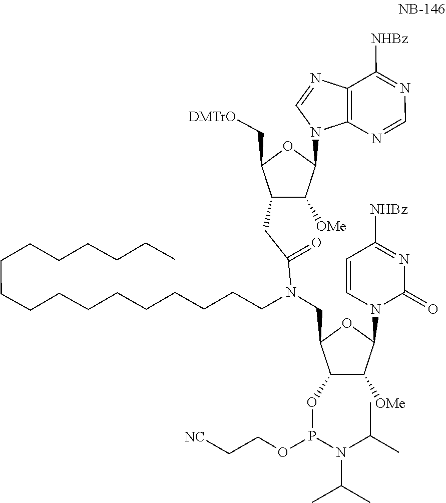

Dinucleotide NB-146 has been synthesized following the procedure described for compound NB-130 (example 20). Mass (m/z 1532, M+Na), ³¹P NMR (202, MHz, DMSO-d6) δ 149.98, 149.73, 149.68, 149.22.

Oligonucleotide Comprising Dinucleotide NB-146 has been Synthesized Using the General Procedure Described in Example 1.

### Example 37: NB-147

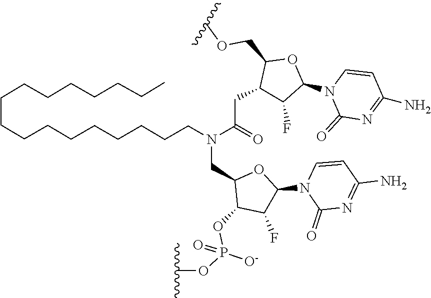 [SMILES: *OC[C@H]1O[C@@H](n2ccc(N)nc2=O)[C@H](F)[C@@H]1CC(=O)N(CCCCCCCCCCCCCCCCC)C[C@H]1O[C@@H](n2ccc(N)nc2=O)[C@H](F)[C@@H]1OP(=O)([O-])O*]

### Synthesis of NB-147: (2R,3R,4R,5R)-2-((2-((2S,3R,4R,5R)-5-(4-benzamido-2-oxopyrimidin-1(2H)-yl)-2-((bis(4-methoxyphenyl)(phenyl)methoxy) methyl)-4-methoxytetrahydrofuran-3-yl)-N-heptadecylacetamido)methyl)-5-(6-benzamido-9H-purin-9-yl)-4-methoxytetrahydrofuran-3-yl(2-cyanoethyl) diisopropylphosphoramidite

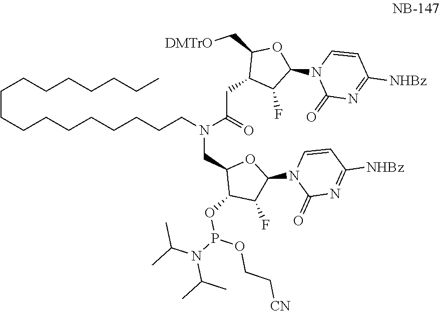

Dinucleotide NB-147 has been synthesized following the procedure described for compound NB-130 (example 20). Mass (m/z 1485, M+Na), ³¹P NMR (202, MHz, DMSO-d6) δ 150.74, 150.35, 150.19, 150.07.

Oligonucleotide Comprising Dinucleotide NB-147 has been Synthesized Using the General Procedure Described in Example 1.

### Example 38: NB-148

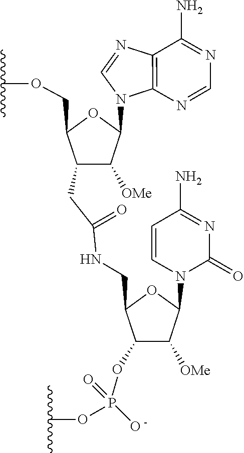 [SMILES: *OC[C@H]1O[C@@H](n2cnc3c(N)ncnc32)[C@H](OC)[C@@H]1CC(=O)NC[C@H]1O[C@@H](n2ccc(N)nc2=O)[C@H](OC)[C@@H]1OP(=O)(O)O*]

### Synthesis of NB-148

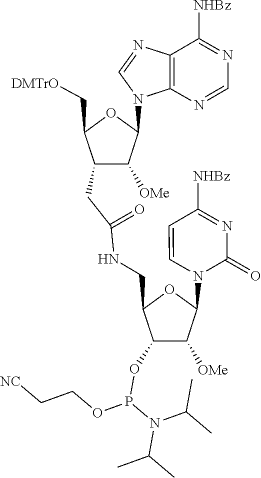

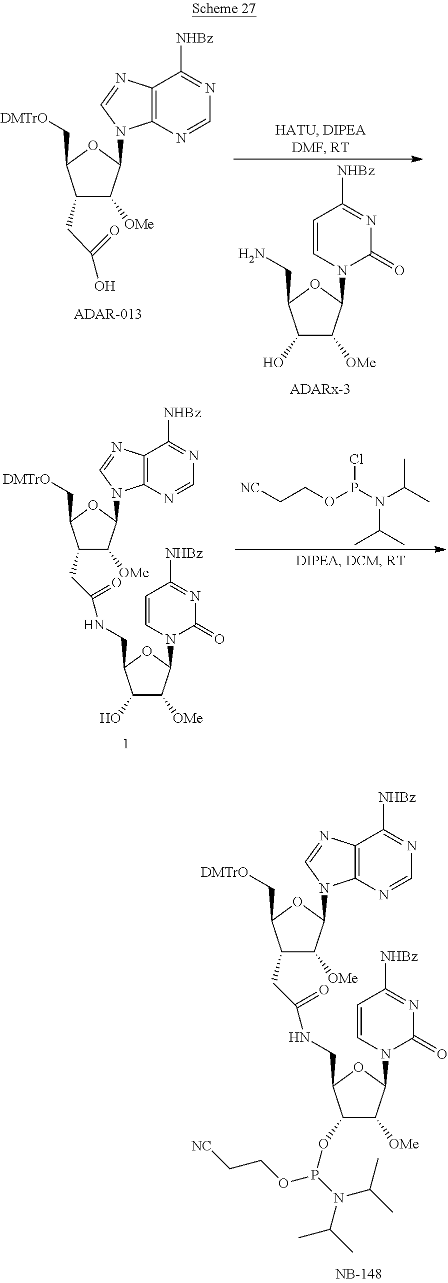

To a stirred solution of acid ADAR-13 (1 g, 1.37 mmol, 1 eq), amine ADARx-3 (0.543 g, 1.5 mmol, 1.1 eq) and HATU (0.782 g, 2 mmol, 1.5 eq) in DMF (10 mL) was added DIPEA (0.7 mL, 4.1 mmol, 3 eq) and the mixture was stirred for 3 h at room temperature, reaction mixture was diluted with saturated NaHCO₃ (50 ml), precipitated solids were filtered and washed with water, the solids were dried and re dissolved in DCM then purified by DCM/MeOH, 0-10% as an eluent, pure fractions were combined and concentrated to obtain amide 1 (1.3 g, 85%) as a beige solid. LCMS m/z 1073 (M+1).

To a stirred solution of alcohol 1 (1.37 g, 1.3 mmol, 1 eq) and diisopropylethylamine (1.36 mL, 7.8 mmol, 6 eq), in DCM (10 mL), was added N, N-diisopropyl chlorophosphoramidite (0.8 mL 3.9 mmol, 3 eq) dropwise. The reaction mixture was stirred at room temperature for 3 h. Reaction mixture was quenched with Aq. Saturated NaHCO₃ solution (50 mL), extracted with DCM (100) ml, washed with brine (50 mL), dried over Na₂SO₄, and evaporated the crude product was loaded on to (pre-equilibrated with 1% Et₃N-EtOAc) Biotage silica gel column (100 g 20 μm), and purified by flash chromatography using EtOAc/MeOH 0-10% 5 CV then 10% 10 CV containing 1% Et₃N as an additive. Pure fractions were combined and concentrated, dried under high vacuum to obtain Phosphoramidite NB-148 (1.2 g, 73%) product as a pale-yellow solid. 95% purity by HPLC, Mass (m/z 1294 M++Na). ³¹P NMR (202, MHz, DMSO-d6) δ 149.97, 149.78.

Oligonucleotide Comprising Dinucleotide NB-148 has been Synthesized Using the General Procedure Described in Example 1.

### Example 39: NB-149

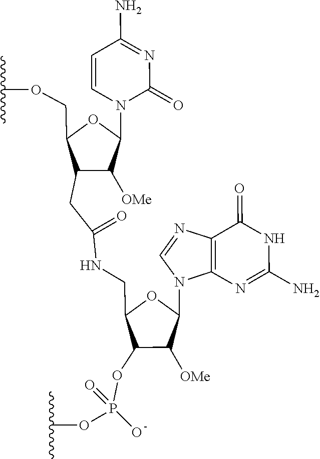

### Synthesis of NB-149

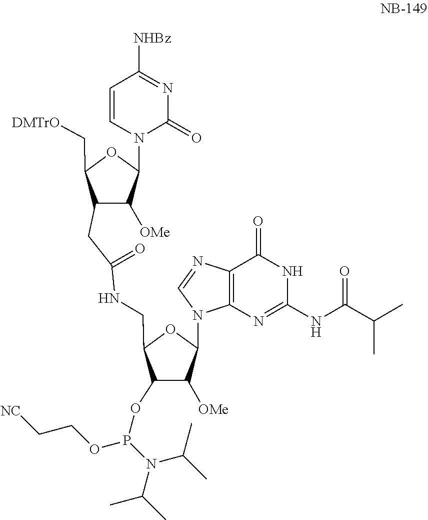

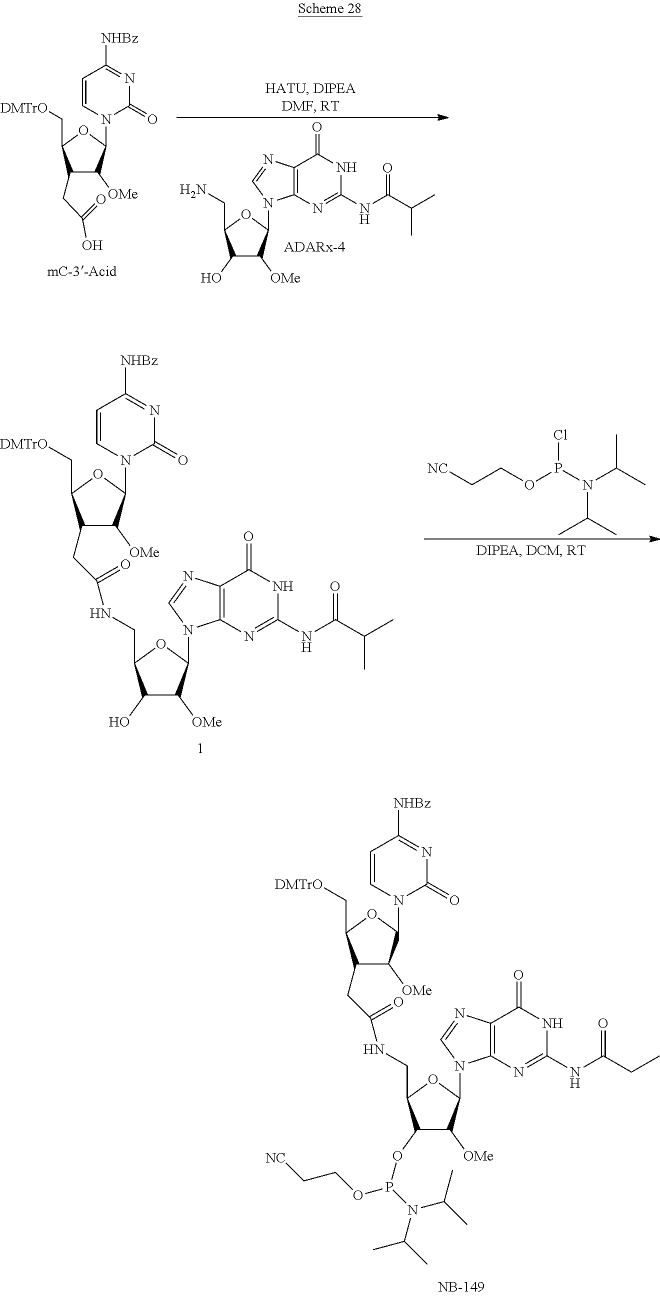

To a stirred solution of mC-3′-acid (1.3 g, 1.844 mmol, 1 eq), amine ADARx-4 (0.776 g, 2.121 mmol, 1.15 eq) and HATU (1 g, 2.7 mmol, 1.5 eq) in DMF (10 mL) was added DIPEA (0.96 mL, 5.53 mmol, 3 eq) and the mixture was stirred for 3 h at room temperature, reaction mixture was added dropwise to a vigorously stirring water (50 ml), precipitated solids were filtered and washed with water, the solids were dried and re dissolved in DCM then purified by DCM/MeOH, 0-5% as an eluent, pure fractions were combined and concentrated to obtain amide 1 (1.4 g, 62%) as a beige solid. LCMS m/z 1055 (M+1).

To a stirred solution of alcohol 1 (1.325 g, 1.257 mmol, 1 eq) and diisopropylethylamine (1.3 mL, 7.54 mmol, 6 eq), in DCM (10 mL), was added N, N-diisopropyl chlorophosphoramidite (0.8 mL 3.7 mmol, 3 eq) dropwise. The reaction mixture was stirred at room temperature for 3 h. Reaction mixture was quenched with Aq. Saturated NaHCO₃ solution (50 mL), extracted with DCM (100) ml, washed with brine (50 mL), dried over Na₂SO₄, and evaporated the crude product was loaded on to (pre-equilibrated with 1% Et₃N-EtOAc) Biotage silica gel column (100 g 20 μm), and purified by flash chromatography using EtOAc/MeOH 0-5% 5 CV then 5% 10 CV containing 1% Et₃N as an additive. Pure fractions were combined and concentrated, dried under high vacuum to obtain Phosphoramidite NB-149 (1.1 g, 69%) as a pale-yellow solid. 95% purity by HPLC, Mass (m/z 1276 M++Na), ³¹P NMR (202, MHz, DMSO-d6) δ 150.38, 150.12, 148.60.

Oligonucleotide Comprising Dinucleotide NB-149 has been Synthesized Using the General Procedure Described in Example 1.

### Example 40: NB-150

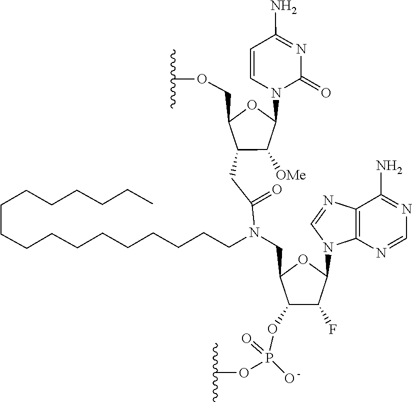 [SMILES: *OC[C@H]1O[C@@H](n2ccc(N)nc2=O)[C@H](OC)[C@@H]1CC(=O)N(CCCCCCCCCCCCCCCCC)C[C@H]1O[C@@H](n2cnc3c(N)ncnc32)[C@H](F)[C@@H]1OP(=O)(O)O*]

### Synthesis of NB-150

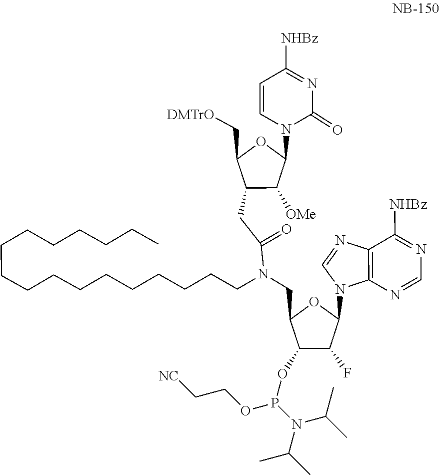

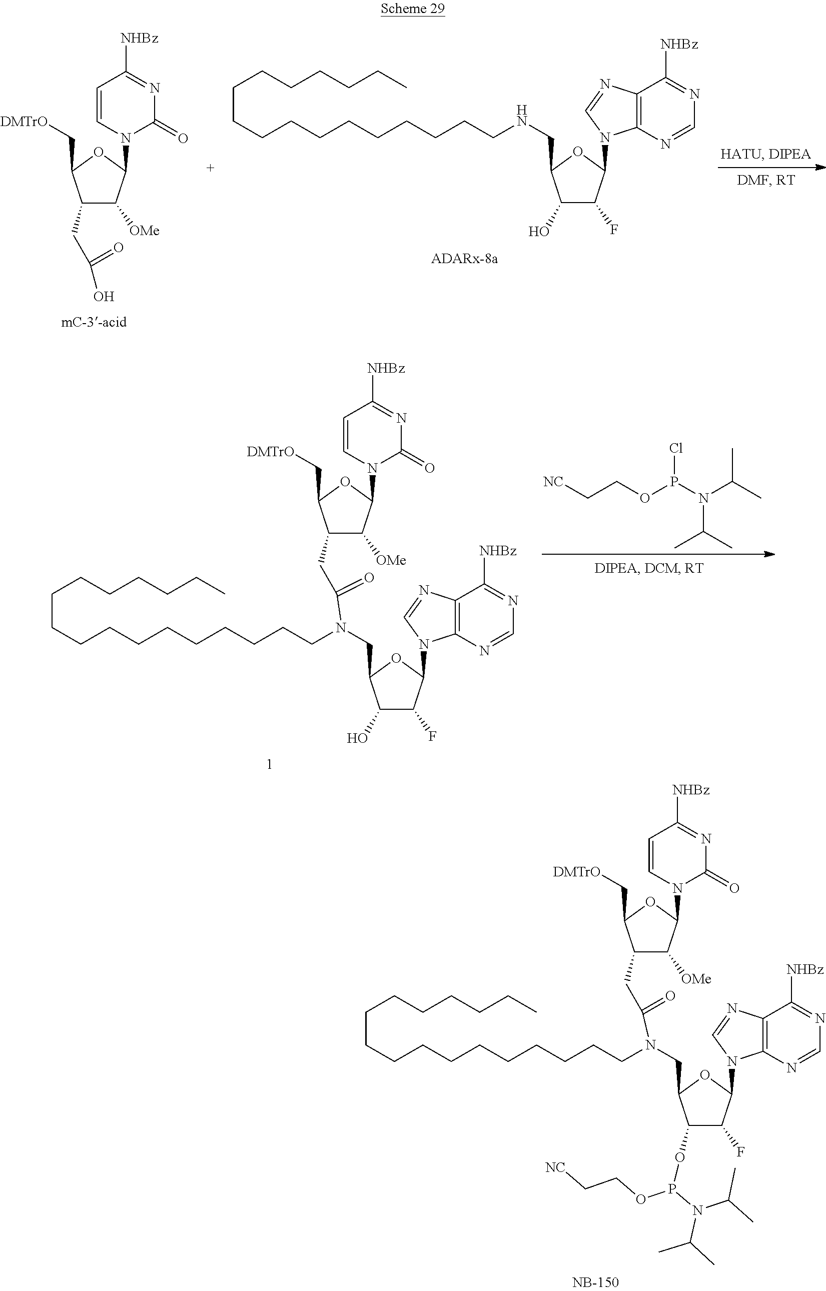

To a stirred solution of mC-3′-acid (1.5 g, 2.128 mmol, 1 eq), amine ADARx-8a (1.493 g, 2.447 mmol, 1.15 eq) and HATU (1.2 g, 3.19 mmol, 1.5 eq) in DMF (20 mL) was added DIPEA (1.11 mL, 6.383 mmol, 3 eq) and the mixture was stirred for 1 h at room temperature, reaction mixture was added dropwise to a vigorously stirring water (100 ml), precipitated solids were filtered and washed with water, the solids were dried and re dissolved in DCM then purified by Ethyl acetate/MeOH, 0-5% as an eluent, pure fractions were combined and concentrated to obtain amide 1 (2.4 g, 75%) as a beige solid. LCMS m/z 1320 (M+Na).

To a stirred solution of alcohol 1 (2.4 g, 1.85 mmol, 1 eq) and diisopropylethylamine (1.93 mL, 11.1 mmol, 6 eq), in DCM (20 mL), was added N, N-diisopropyl chlorophosphoramidite (1.23 mL 5.54 mmol, 3 eq) dropwise. The reaction mixture was stirred at room temperature for 3 h. Reaction mixture was quenched with Aq. Saturated NaHCO₃ solution (50 mL), extracted with DCM (100) ml, washed with brine (50 mL), dried over Na₂SO₄, and evaporated the crude product was loaded on to (pre-equilibrated with 1% Et₃N-EtOAc) Biotage silica gel column (100 g 20 μm), and purified by flash chromatography using EtOAc/MeOH 0-3% 5 CV then 3% 10 CV containing 1% Et₃N as an additive. Pure fractions were combined and concentrated, dried under high vacuum to obtain Phosphoramidite NB-150 (2.1 g, 76%) as an off-white solid. 95% purity by HPLC, 89% purity by P31-NMR. Mass (m/z 1521 M++Na). ³¹P NMR (202, MHz, DMSO-d6) δ 150.16, 150.13, 149.98, 149.94, 149.78, 149.75.

Oligonucleotide Comprising Dinucleotide NB-150 has been Synthesized Using the General Procedure Described in Example 1.

### Example 41: NB-151

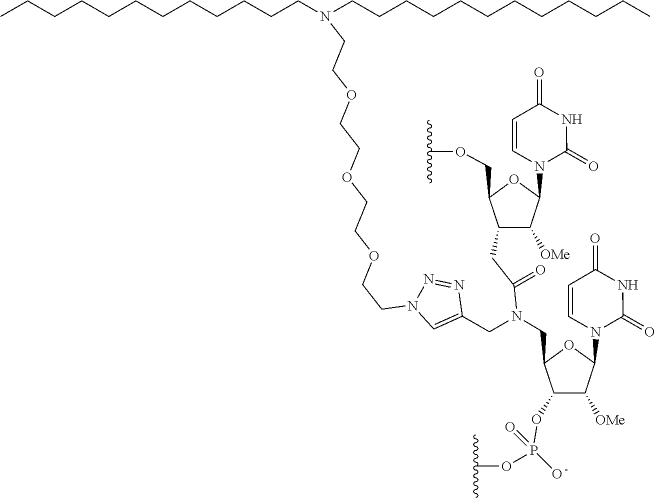 [SMILES: *OC[C@H]1O[C@@H](n2ccc(=O)[nH]c2=O)[C@H](OC)[C@@H]1CC(=O)N(Cc1cn(CCOCCOCCOCCN(CCCCCCCCCCCC)CCCCCCCCCCCC)nn1)C[C@H]1O[C@@H](n2ccc(=O)[nH]c2=O)[C@H](OC)[C@@H]1OP(=O)(O)O*]

### Synthesis of NB-151

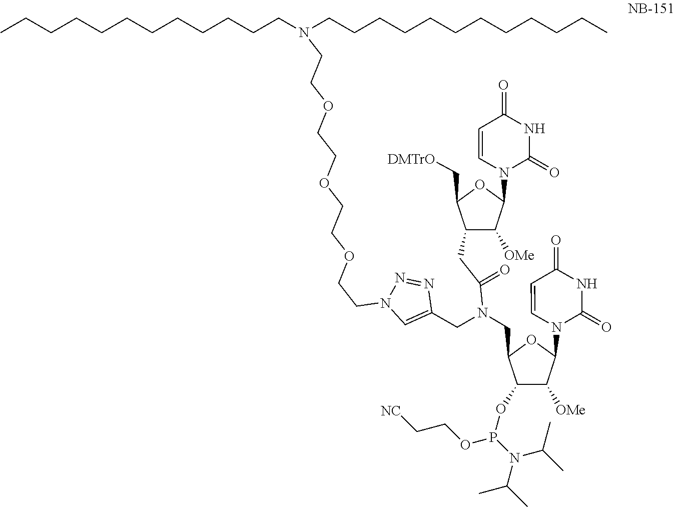

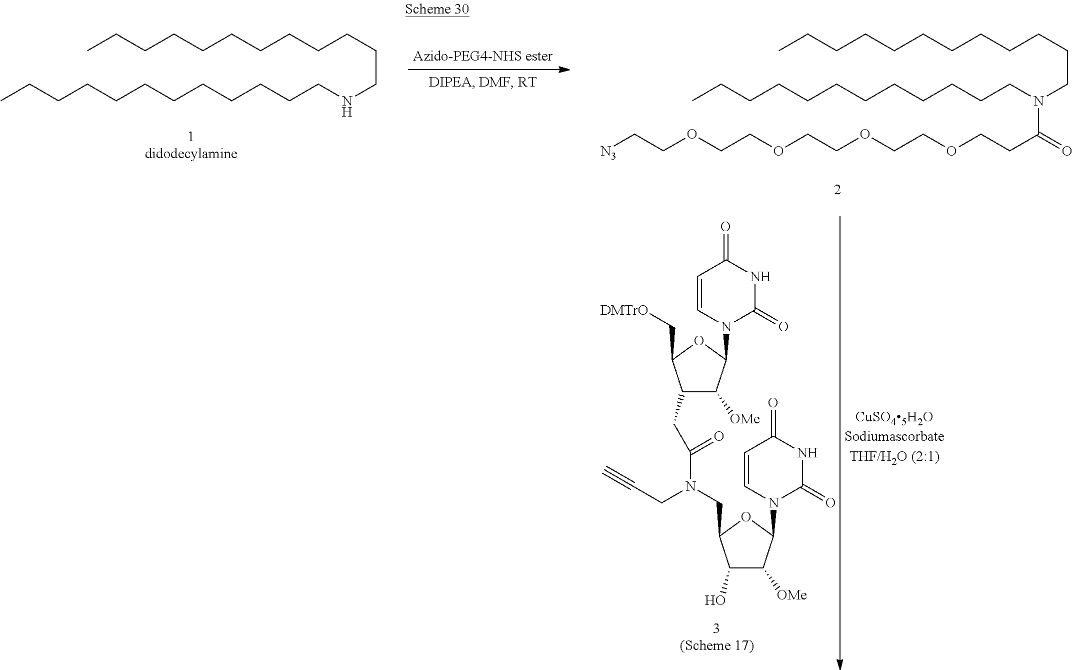

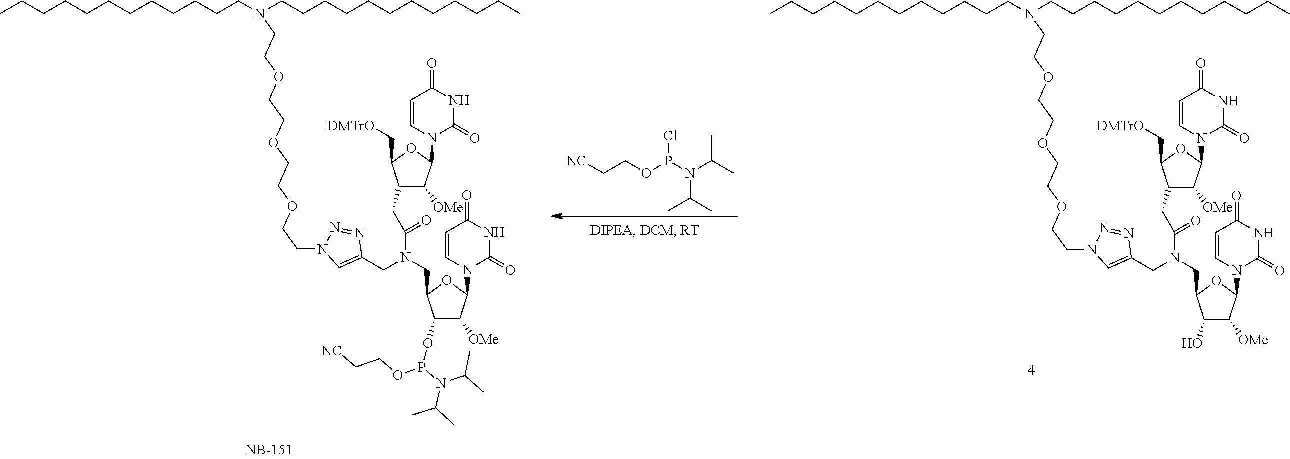

To a stirred solution of didodecylamine (500 mg, 1.416 mmol, 1 eq), and DIPEA (0.49 mL, 2.83 mmol, 2 eq) in DMF (2 mL) was added Azido-PEG4-NHS ester (605 mg, 1.558 mmol, 1.1 eq). The resulting mixture was stirred for 12 h. at RT. LCMS showed amide formation. Reaction mixture was concentrated, the crude was purified by DCM/MeOH, 0-10% as an eluent, pure fractions were combined and concentrated to obtain amide (305 mg, 34%) as a beige solid. LCMS m/z 627 (M+1).

To a solution of alkyne 3 (0.43 g, 0.489 mmol, 1 eq) and azide (298 mg, 0.538 mmol, 1 eq) in THF (10 mL) at RT was added a solution of CuSO₄·5H₂O (147 mg, 0.587 mmol, 1.2 eq) in water (1 mL) followed by a solution of sodium ascorbate (145 mg, 0.171 mmol, 1.5 eq) in water (1 mL). The reaction was stirred for 12 hrs at RT. Then the reaction was diluted with EtOAc 50 mL and washed with NaHCO₃20 mL), dried over Na₂SO₄, filtered, and concentrated. The residue was purified by 0-10% EtOAc/MeOH to give 0.400 g of triazole 4 (81%) as a beige solid, mass m/z=1434 (M+).

To a stirred solution of alcohol 4 (0.425 g, 0.296 mmol, 1 eq) and diisopropylethylamine (0.31 mL, 1.77 mmol, 6 eq), in DCM (10 mL), was added N, N-diisopropyl chlorophosphoramidite (0.2 mL 0.9 mmol, 3 eq) dropwise. The reaction mixture was stirred at room temperature for 3 h. Reaction mixture was quenched with Aq. Saturated NaHCO₃ solution (50 mL), extracted with DCM (50) ml, washed with brine (50 mL), dried over Na₂SO₄, and evaporated the crude product was loaded on to (pre-equilibrated with 1% Et₃N-EtOAc) Biotage silica gel column (25 g 20 μm), and purified by flash chromatography using EtOAc/MeOH 0-5% 5 CV then 5% 10 CV containing 1% Et₃N as an additive. Pure fractions were combined and concentrated, dried under high vacuum to obtain Phosphoramidite NB-151 (0.4 g, 82%) as a white solid. 95% purity by HPLC, Mass (m/z 1635 M+). ³¹P NMR (202, MHz, DMSO-d6) δ 149.92, 149.72, 149.98, 149.58, 149.56, 147.98, 147.77, 147.71.

Oligonucleotide Comprising Dinucleotide NB-151 has been Synthesized Using the General Procedure Described in Example 1.

### Example 42: NB-152

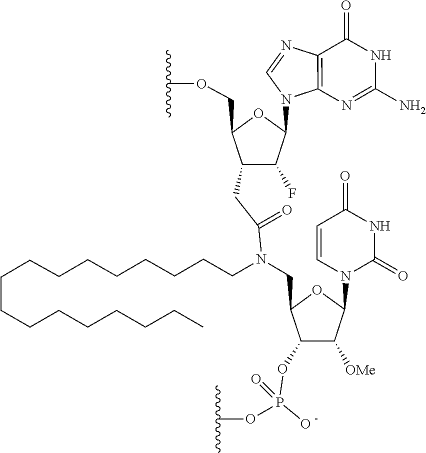 [SMILES: *OC[C@H]1O[C@@H](n2cnc3c(=O)[nH]c(N)nc32)[C@H](F)[C@@H]1CC(=O)N(CCCCCCCCCCCCCCCCC)C[C@H]1O[C@@H](n2ccc(=O)[nH]c2=O)[C@H](OC)[C@@H]1OP(=O)(O)O*]

### Synthesis of NB-152

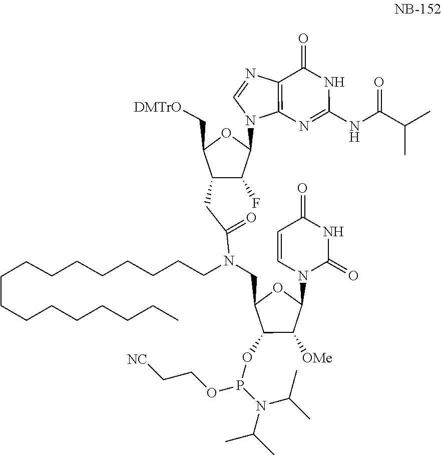

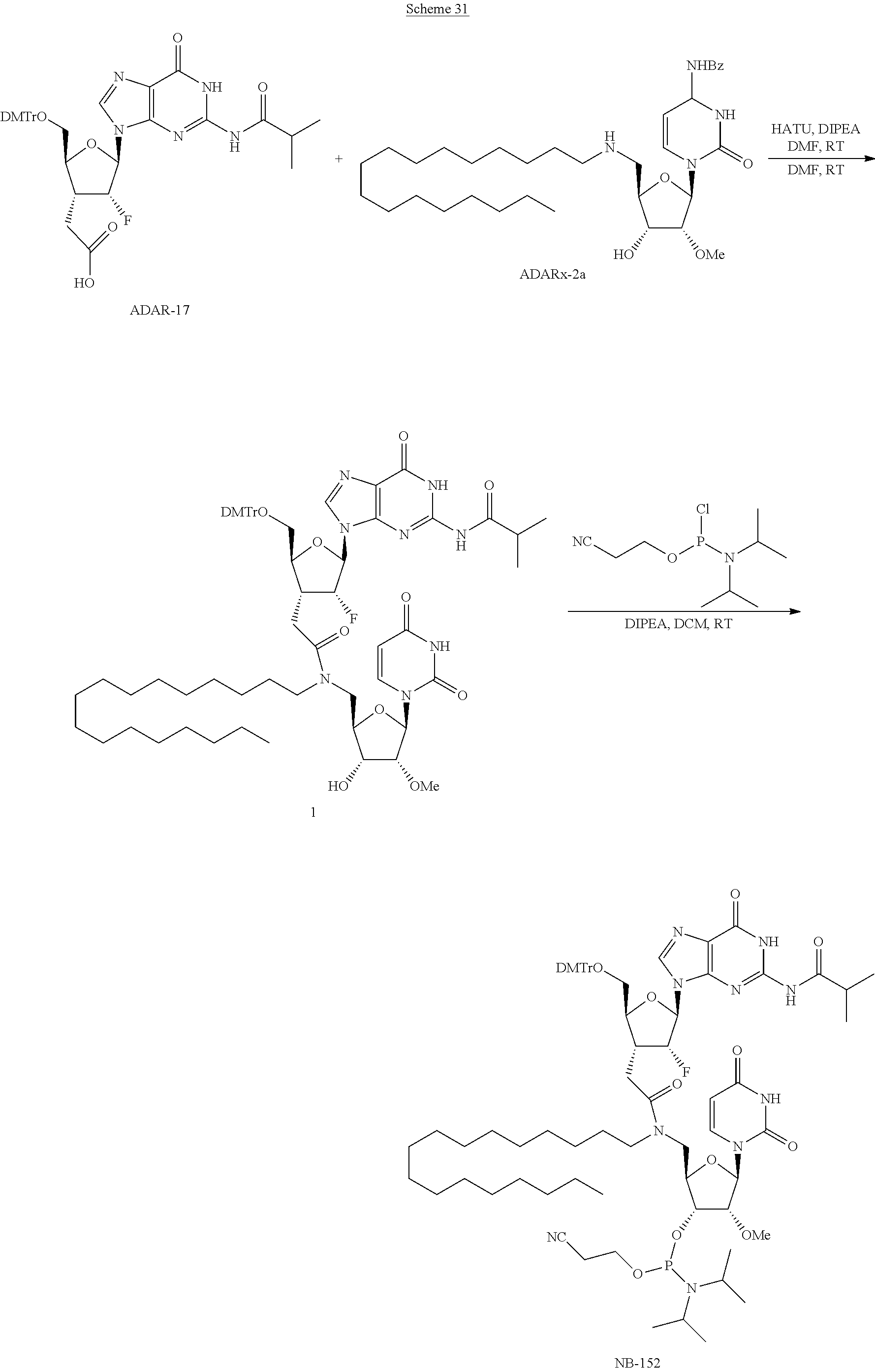

To a stirred solution of acid ADAR-17 (3.5 g, 5.0 mmol, 1 eq), amine ADARx-2a (2.85 g, 5.75 mmol, 1.15 eq) and HATU 2.85 g, 7.51 mmol, 1.5 eq) in DMF (25 mL) was added DIPEA (2.6 mL, 15.0 mmol, 3 eq) and the mixture was stirred for 2 h at room temperature, reaction mixture was added dropwise to a vigorously stirring solution 1:1 water/Aq. saturated NaHCO₃ (200 ml), precipitated solids were filtered and washed with water, the solids were dried and re dissolved in EtOAc then purified by flash chromatography (100 g 20 micron Biotage column) using MeOH/EtOAc, 0-5% 5 CV then 5-5% 10 CV as an eluent, pure fractions were combined and concentrated to obtain amide (4.3 g, 63%) as a beige solid. NMR and LCMS m/z 1178 (M+1) are corresponding with the product. To a stirred solution of alcohol (4.2 g, 0.3.56 mmol, 1 eq) and diisopropylethylamine (3.72 mL, 21.41 mmol, 6 eq), in DCM (25 mL), was added N, N-diisopropyl chlorophosphoramidite (2.38 mL 10.7 mmol, 3 eq) dropwise. The reaction mixture was stirred at room temperature for 3 h. Reaction mixture was quenched with Aq. Saturated NaHCO₃ solution (100 mL), extracted with DCM (100) ml, washed with brine (100 mL), dried over Na₂SO₄, and evaporated the crude product was loaded on to (pre-equilibrated with 1% Et₃N-EtOAc) Biotage silica gel column (100 g 20 μm), and purified by flash chromatography using EtOAc/MeOH 0-0 1 CV, 0-5% 5 CV then 5% 10 CV containing 1% Et₃N as an additive. Pure fractions were combined and concentrated, dried under high vacuum to obtain Phosphoramidite product Batch-1 (2 g), also obtained 4 g of mixture with reagent, the mixture was further purified by flash chromatography using EtOAc/MeOH 0-0 1 CV, 0-1% 5 CV then 1% 10 CV containing 1% Et₃N as an additive. Pure fractions were combined and concentrated, dried under high vacuum to obtain Phosphoramidite NB-152 (1.65 g) both batches were combined to get total 3.65 g, 74% as a white solid. 95% purity by HPLC, Mass (m/z 1399 M++Na), ³¹P NMR (202, MHz, DMSO-d6) δ 149.86, 149.68, 149.48, 149.29.

Oligonucleotide Comprising Dinucleotide NB-152 has been Synthesized Using the General Procedure Described in Example 1.

### Example 43: NB-153

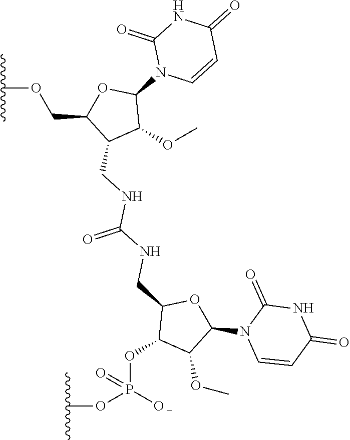 [SMILES: *OC[C@H]1O[C@@H](n2ccc(=O)[nH]c2=O)[C@H](OC)[C@@H]1CNC(=O)NC[C@H]1O[C@@H](n2ccc(=O)[nH]c2=O)[C@H](OC)[C@@H]1OP(=O)(O)O*]

### Synthesis of NB-153: (2R,3R,4R,5R)-2-((3-(((2S,3R,4R,5R)-2-((bis(4-methoxyphenyl)(phenyl)methoxy)methyl)-5-(2,4-dioxo-3,4-dihydropyrimidin-1(2H)-yl)-4-methoxytetrahydrofuran-3-yl)methyl)ureido)methyl)-5-(2,4-dioxo-3,4-dihydropyrimidin-1(2H)-yl)-4-methoxytetrahydrofuran-3-yl (2-cyanoethyl) diisopropylphosphoramidite

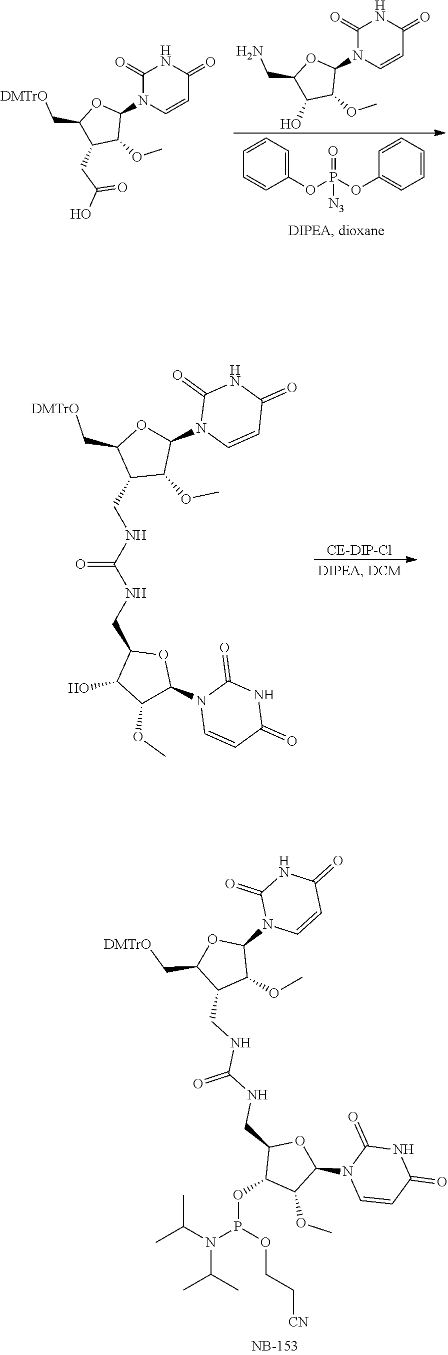

### Step 1: 1-(((2S,3R,4R,5R)-2-((bis(4-methoxyphenyl)(phenyl)methoxy)methyl)-5-(2,4-dioxo-3,4-dihydropyrimidin-1(2H)-yl)-4-methoxytetrahydrofuran-3-yl)methyl)-3-(((2R,3R,4R,5R)-5-(2,4-dioxo-3,4-dihydropyrimidin-1(2H)-yl)-3-hydroxy-4-methoxytetrahydrofuran-2-yl)methyl)urea

The acid, 2-((2S,3R,4R,5R)-2-((bis(4-methoxyphenyl)(phenyl)methoxy)methyl)-5-(2,4-dioxo-3,4-dihydropyrimidin-1(2H)-yl)-4-methoxytetrahydrofuran-3-yl)acetic acid (0.25 g, 0.25 mmol) was suspended in dioxane (3 mL), treated with DIPEA (0.11 mL, 0.6 mmol) and diphenylphosphoryl azide (0.12 mL, 0.5 mmol). The mixture was stirred at 50° C. for 3 hours. The mixture was cooled to 20° C. and the amine, 1-((2R,3R,4R,5R)-5-(aminomethyl)-4-hydroxy-3-methoxytetrahydrofuran-2-yl)pyrimidine-2,4(1H,3H)-dione (0.13 g, 0.5 mmol) was added in one portion followed by another aliquot of DIPEA (0.11 mL, 0.6 mmol). The mixture was stirred at 50° C. until the mixture became homogenous. The reaction mixture was concentrated to dryness. The residue was purified by silica gel chromatography (40 to 100% ethyl acetate in hexanes, followed 0 to 25% methanol in ethyl acetate; Biotage 25 g column) to afford the desired product, followed by titration with DCM/hexane to afford the titled compound (334 mg, 94% yield) as a white solid. MS (ESI, negative mode) m/z=901.5[M+HCO₂]⁻, 855.6 [M−H]⁻.

### Step 2: (2R,3R,4R,5R)-2-((3-(((2S,3R,4R,5R)-2-((bis(4-methoxyphenyl)(phenyl) methoxy)methyl)-5-(2,4-dioxo-3,4-dihydropyrimidin-1(2H)-yl)-4-methoxytetrahydrofuran-3-yl)methyl)ureido)methyl)-5-(2,4-dioxo-3,4-dihydropyrimidin-1(2H)-yl)-4-methoxytetrahydrofuran-3-yl (2-cyanoethyl) diisopropylphosphoramidite

To a stirred solution of alcohol 1-(((2S,3R,4R,5R)-2-((bis(4-methoxyphenyl)(phenyl) methoxy)methyl)-5-(2,4-dioxo-3,4-dihydropyrimidin-1(2H)-yl)-4-methoxytetrahydrofuran-3-yl)methyl)-3-(((2R,3R,4R,5R)-5-(2,4-dioxo-3,4-dihydropyrimidin-1(2H)-yl)-3-hydroxy-4-methoxytetrahydrofuran-2-yl)methyl)urea (0.74 g, 0.86 mmol) and diisopropylethylamine (0.9 mL, 5.2 mmol) in DCM (15 mL), under an atmosphere of argon, was added N,N-diisopropyl chlorophosphoramidite (0.58 mL, 2.6 mmol) dropwise. The reaction mixture was stirred at room temperature overnight. The reaction mixture was quenched with saturated NaHCO₃ solution (30 mL), extracted with DCM (2×100 mL), washed with brine (50 mL), dried over Na₂SO₄, and concentrated. The crude was loaded onto a 25 g Biotage silica gel column (pre-equilibrated 1% Et₃N in heptane) using DCM (0-100% ethyl acetate/heptane w/1% each of Et₃N, followed by 0-20% methanol in 1% Et₃N in ethyl acetate) to obtain the titled compound NB-153 (475 mg, 52% yield). ³¹P NMR (202, MHz, DMSO-d₆) δ 150.11, 149.64. MS (ESI): m/z=1079.1 [M+Na]⁺.

Oligonucleotide Comprising Dinucleotide NB-153 has been Synthesized Using the General Procedure Described in Example 1.

### Example 44: NB-154

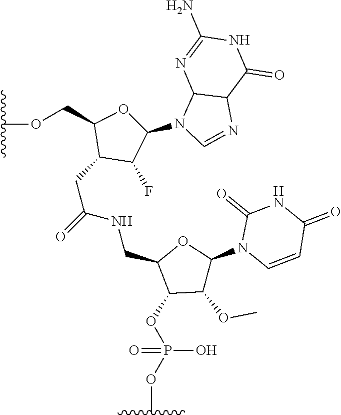 [SMILES: *OC[C@H]1O[C@@H](N2C=NC3C(=O)NC(N)=NC32)[C@H](F)[C@@H]1CC(=O)NC[C@H]1O[C@@H](n2ccc(=O)[nH]c2=O)[C@H](OC)[C@@H]1OP(=O)(O)O*]

### Synthesis of NB-154: (2R,3R,4R,5R)-2-((2-((2S,3R,4R,5R)-2-((bis(4-methoxyphenyl)(phenyl)methoxy)methyl)-4-fluoro-5-(2-isobutyramido-6-oxo-1,6-dihydro-9H-purin-9-yl)tetrahydrofuran-3-yl)acetamido) methyl)-5-(2,4-dioxo-3,4-dihydropyrimidin-1(2H)-yl)-4-methoxytetrahydrofuran-3-yl(2-cyanoethyl) diisopropylphosphoramidite

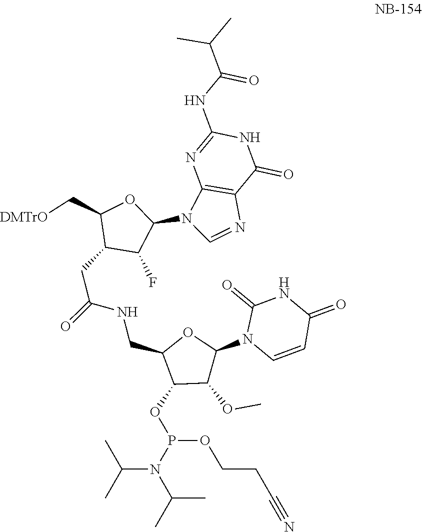

Dinucleotide NB-154 has been synthesized following the procedure described for compound NB-131 (example 21). Mass, 1138.9 [M+H]⁺, ³¹P NMR (202 MHz, DMSO-d₆) δ 149.97, 149.55.

Oligonucleotide Comprising Dinucleotide NB-154 has been Synthesized Using the General Procedure Described in Example 1.

### Example 45: NB-155

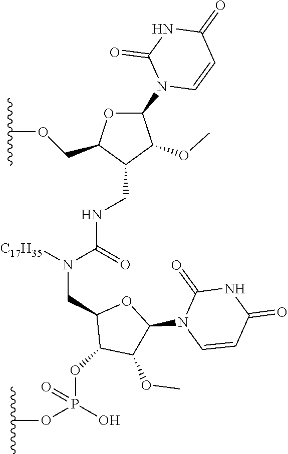

### Synthesis of NB-155: (2R,3R,4R,5R)-2-((3-(((2S,3R,4R,5R)-2-((bis(4-methoxyphenyl)(phenyl)methoxy)methyl)-5-(2,4-dioxo-3,4-dihydropyrimidin-1(2H)-yl)-4-methoxytetrahydrofuran-3-yl)methyl)-1-heptadecylureido) methyl)-5-(2,4-dioxo-3,4-dihydropyrimidin-1(2H)-yl)-4-methoxytetrahydrofuran-3-yl (2-cyanoethyl) diisopropylphosphoramidite

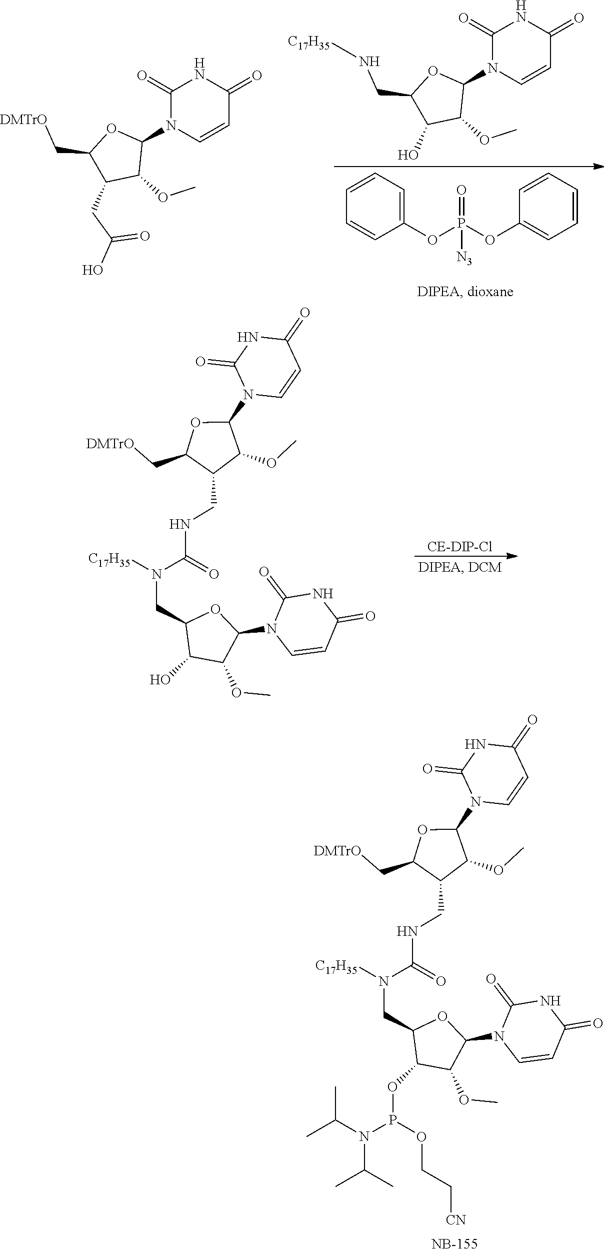

### Step 1: 3-(((2S,3R,4R,5R)-2-((bis(4-methoxyphenyl)(phenyl)methoxy) methyl)-5-(2,4-dioxo-3,4-dihydropyrimidin-1(2H)-yl)-4-methoxytetrahydrofuran-3-yl)methyl)-1-(((2R,3R,4R,5R)-5-(2,4-dioxo-3,4-dihydropyrimidin-1(2H)-yl)-3-hydroxy-4-methoxytetrahydrofuran-2-yl)methyl)-1-heptadecylurea

The acid, 2-((2S,3R,4R,5R)-2-((bis(4-methoxyphenyl)(phenyl)methoxy) methyl)-5-(2,4-dioxo-3,4-dihydropyrimidin-1(2H)-yl)-4-methoxytetrahydrofuran-3-yl)acetic acid (0.15 g, 0.25 mmol) was suspended in dioxane (3 mL) and treated with DIPEA (0.065 mL, 0.37 mmol) and diphenylphosphoryl azide (0.07 mL, 0.3 mmol). The mixture was stirred at 50° C. for 3 h. The mixture was cooled to 20° C. and the amine, 1-((2R,3R,4R,5R)-5-((heptadecylamino) methyl)-4-hydroxy-3-methoxytetrahydrofuran-2-yl)pyrimidine-2,4(1H,3H)-dione (0.14 g, 0.3 mmol) was added followed by another aliquot of DIPEA (0.065 mL, 0.37 mmol). The mixture was stirred at 50° C. until the mixture became homogenous. The mixture was concentrated to dryness. The residue was purified with silica gel chromatography (40 to 100% ethyl acetate in hexanes, followed 0 to 20% methanol in ethyl acetate; Biotage 25 g column) to afford the titled compound (224 mg, 82% yield) as a white solid. MS (ESI): m/z=1117.9 [M+Na]⁺.

### Step 2: (2R,3R,4R,5R)-2-((3-(((2S,3R,4R,5R)-2-((bis(4-methoxyphenyl) (phenyl)methoxy)methyl)-5-(2,4-dioxo-3,4-dihydropyrimidin-1(2H)-yl)-4-methoxytetrahydrofuran-3-yl)methyl)-1-heptadecylureido) methyl)-5-(2,4-dioxo-3,4-dihydropyrimidin-1(2H)-yl)-4-methoxytetrahydrofuran-3-yl (2-cyanoethyl) diisopropylphosphoramidite

To a stirred solution of alcohol, 3-(((2S,3R,4R,5R)-2-((bis(4-methoxyphenyl)(phenyl)methoxy)methyl)-5-(2,4-dioxo-3,4-dihydropyrimidin-1(2H)-yl)-4-methoxytetrahydrofuran-3-yl)methyl)-1-(((2R,3R,4R,5R)-5-(2,4-dioxo-3,4-dihydropyrimidin-1(2H)-yl)-3-hydroxy-4-methoxytetrahydrofuran-2-yl)methyl)-1-heptadecylurea (1.78 g, 1.47 mmol) and diisopropylethylamine (1.5 mL, 8.8 mmol) in DCM (15 mL), under an atmosphere of argon, was added N,N-diisopropyl chlorophosphoramidite (1.0 mL, 8.3 mmol) dropwise. The reaction mixture was stirred at room temperature for 1 h. The reaction mixture was washed with sat. NaHCO₃ (20 mL), dried over Na₂SO₄ and concentrated (to <5 mL). It was treated with hexanes (70 mL) to obtain a white semi-solid. The supernatant was decanted off and the solid were dissolved in DCM and loaded onto a Biotage 25 g silica column (pre-equilibrated with 1% Et₃N-hexanes) and purified using an ABC gradient (A=1% Et₃N in hexanes; B=1% Et₃N in ethyl acetate; C=MeOH; 0-100% A in B; 0-10%-35% C in B) to obtain the crude material. It was redissolved in DCM (20 mL) and reconcentrated twice to remove residual methanol. It was dissolved in DCM (5-10 mL). Hexanes were added slowly, in portions, with swirling, until a white solid was obtained. The mixture was sonicated and swirled to precipitate the product. The turbid supernatant was decanted off. The solid was washed with more hexanes and dried under vacuum overnight to afford the titled compound NB-155 (828 mg, 78% yield) as a white solid. ³¹P NMR (202 MHz, DMSO-d₆) δ 149.89, 149.73. MS(ESI): m/z=1317.3 [M+Na]⁺.

Oligonucleotide Comprising Dinucleotide NB-155 has been Synthesized Using the General Procedure Described in Example 1.

### Example 46: NB-156

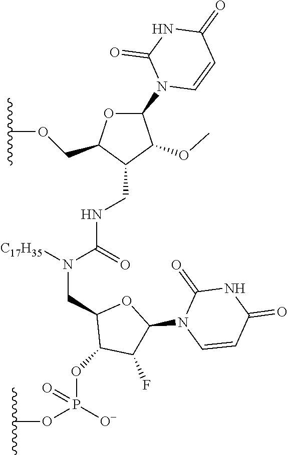

### Synthesis: NB-156: (2R,3R,4R,5R)-2-((3-(((2S,3R,4R,5R)-2-((bis(4-methoxyphenyl)(phenyl)methoxy)methyl)-5-(2,4-dioxo-3,4-dihydropyrimidin-1(2H)-yl)-4-methoxytetrahydrofuran-3-yl)methyl)-1-heptadecylureido) methyl)-5-(2,4-dioxo-3,4-dihydropyrimidin-1(2H)-yl)-4-fluorotetrahydrofuran-3-yl (2-cyanoethyl) diisopropylphosphoramidite

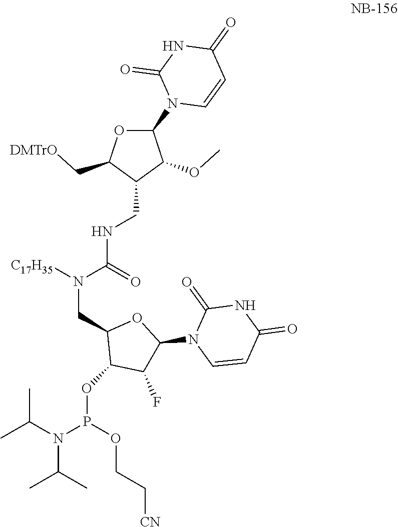

NB-156 was made using the same method as in NB-155 by replacing the amine with 1-((2R,3R,4R,5R)-3-fluoro-5-((heptadecylamino)methyl)-4-hydroxytetrahydrofuran-2-yl)pyrimidine-2,4(1H,3H)-dione in step 1 to afford the titled compound NB-156 as a white solid. ³¹P NMR (202 MHz, DMSO-d₆) δ 150.15, 150.09, 150.04, 150.00. MS(ESI): m/z=1305.2 [M+Na]⁺.

Oligonucleotide Comprising Dinucleotide NB-156 has been Synthesized Using the General Procedure Described in Example 1.

### Example 47: NB-157

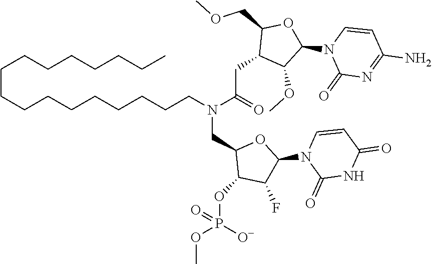 [SMILES: CCCCCCCCCCCCCCCCCN(C[C@H]1O[C@@H](n2ccc(=O)[nH]c2=O)[C@H](F)[C@@H]1OP(=O)([O-])OC)C(=O)C[C@H]1[C@@H](OC)[C@H](n2ccc(N)nc2=O)O[C@@H]1COC]

### Synthesis of NB-157: (2R,3R,4R,5R)-2-((2-((2S,3R,4R,5R)-5-(4-benzamido-2-oxopyrimidin-1(2H)-yl)-2-((bis(4-methoxyphenyl)(phenyl)methoxy) methyl)-4-methoxytetrahydrofuran-3-yl)-N-heptadecylacetamido)methyl)-5-(2,4-dioxo-3,4-dihydropyrimidin-1(2H)-yl)-4-fluorotetrahydrofuran-3-yl (2-cyanoethyl) diisopropylphosphoramidite

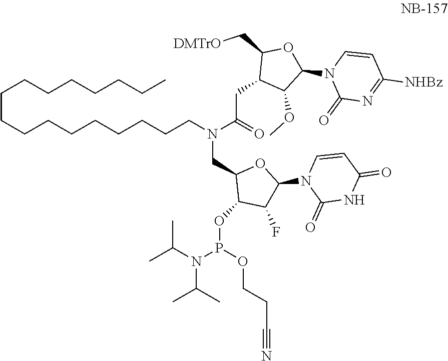

Dinucleotide NB-157 has been synthesized following the procedure described for compound NB-130 (example 20). Mass, 1393.4 [M+Na]⁺, ³¹P NMR (202 MHz, DMSO-d₆) δ 150.19, 150.14, 150.10, 150.06, 149.77, 149.73.

Oligonucleotide Comprising Dinucleotide NB-157 has been Synthesized Using the General Procedure Described in Example 1.

### Example 48: NB-158

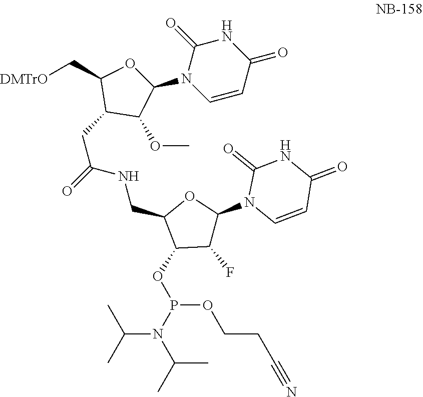

### Synthesis of NB-158: (2R,3R,4R,5R)-2-((2-((2S,3R,4R,5R)-2-((bis(4-methoxyphenyl)(phenyl)methoxy)methyl)-5-(2,4-dioxo-3,4-dihydropyrimidin-1(2H)-yl)-4-methoxytetrahydrofuran-3-yl)acetamido) methyl)-5-(2,4-dioxo-3,4-dihydropyrimidin-1(2H)-yl)-4-fluorotetrahydrofuran-3-yl(2-cyanoethyl) diisopropylphosphoramidite

Dinucleotide NB-158 has been synthesized following the procedure described for compound NB-131 (example 21). Mass, 1052 [M+Na]⁺, ³¹P NMR (202 MHz, DMSO-d₆) δ 150.45, 150.42, 150.04, 149.97.

Oligonucleotide Comprising Dinucleotide NB-158 has been Synthesized Using the General Procedure Described in Example 1.

### Example 49: NB-159

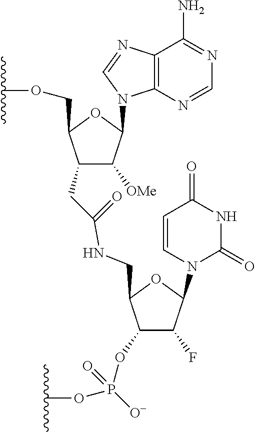 [SMILES: *OC[C@H]1O[C@@H](n2cnc3c(N)ncnc32)[C@H](OC)[C@@H]1CC(=O)NC[C@H]1O[C@@H](n2ccc(=O)[nH]c2=O)[C@H](F)[C@@H]1OP(=O)([O-])O*]

### Synthesis of NB-159: (2R,3R,4R,5R)-2-((2-((2S,3R,4R,5R)-5-(6-benzamido-9H-purin-9-yl)-2-((bis(4-methoxy phenyl)(phenyl)methoxy)methyl)-4-methoxy tetrahydrofuran-3-yl)acetamido)methyl)-5-(2,4-dioxo-3,4-dihydropyrimidin-1(2H)-yl)-4-fluorotetrahydrofuran-3-yl(2-cyanoethyl) diisopropylphosphoramidite

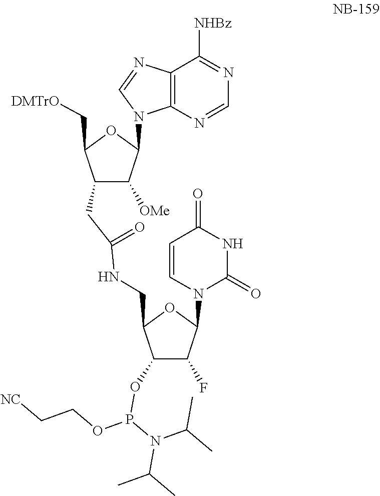

Dinucleotide NB-159 has been synthesized following the procedure described for compound NB-131 (example 21). Mass, 1179.1 [M+Na]⁺, ³¹P NMR (202 MHz, DMSO-d₆) δ 150.47, 150.45, 150.00, 149.93.

Oligonucleotide Comprising Dinucleotide NB-159 has been Synthesized Using the General Procedure Described in Example 1.

### Example 50: NB-160

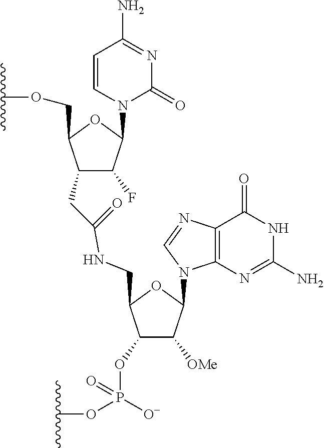 [SMILES: *OC[C@H]1O[C@@H](n2ccc(N)nc2=O)[C@H](F)[C@@H]1CC(=O)NC[C@H]1O[C@@H](n2cnc3c(=O)[nH]c(N)nc32)[C@H](OC)[C@@H]1OP(=O)([O-])O*]

### Synthesis of NB-160: (2R,3R,4R,5R)-2-((2-((2S,3R,4R,5R)-5-(4-benzamido-2-oxopyrimidin-1(2H)-yl)-2-((bis(4-methoxyphenyl)(phenyl)methoxy)methyl)-4-fluorotetrahydrofuran-3-yl)acetamido)methyl)-5-(2-isobutyramido-6-oxo-1,6-dihydro-9H-purin-9-yl)-4-methoxytetrahydrofuran-3-yl(2-cyanoethyl) diisopropylphosphoramidite

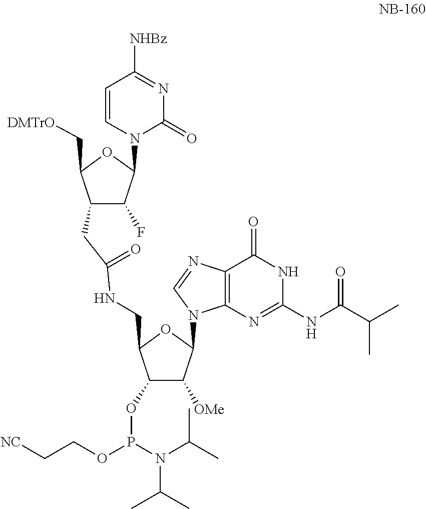

Dinucleotide NB-160 has been synthesized following the procedure described for compound NB-131 (example 21). Mass, 1264.1 [M+Na]⁺, ³¹P NMR (202 MHz, DMSO-d₆) δ 150.56, 150.17.

Oligonucleotide Comprising Dinucleotide NB-160 has been Synthesized Using the General Procedure Described in Example 1.

### Example 51: NB-161

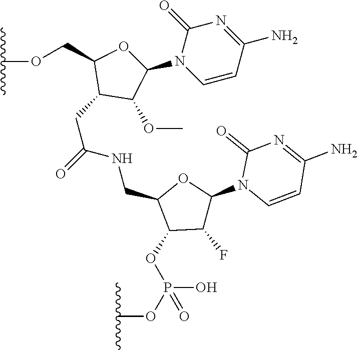 [SMILES: *OC[C@H]1O[C@@H](n2ccc(N)nc2=O)[C@H](OC)[C@@H]1CC(=O)NC[C@H]1O[C@@H](n2ccc(N)nc2=O)[C@H](F)[C@@H]1OP(=O)(O)O*]

### Synthesis of NB-161: (2R,3R,4R,5R)-5-(4-benzamido-2-oxopyrimidin-1(2H)-yl)-2-((2-((2S,3R,4R,5R)-5-(4-benzamido-2-oxopyrimidin-1(2H)-yl)-2-((bis(4-methoxy phenyl)(phenyl)methoxy)methyl)-4-methoxy tetrahydrofuran-3-yl)acetamido)methyl)-4-fluorotetrahydrofuran-3-yl(2-cyanoethyl) diisopropylphosphoramidite

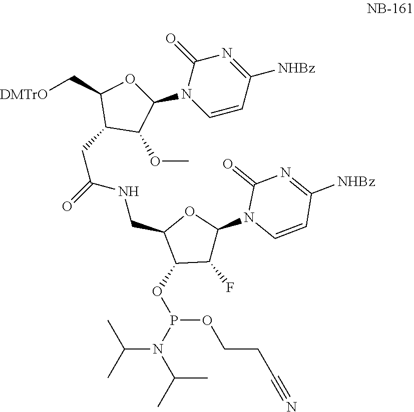

Dinucleotide NB-161 has been synthesized following the procedure described for compound NB-131 (example 21). Mass, 1234.6 [M−H]⁻, ³¹P NMR (202 MHz, DMSO-d₆) δ 150.61, 150.56.

Oligonucleotide Comprising Dinucleotide NB-161 has been Synthesized Using the General Procedure Described in Example 1.

### Example 52: NB-162

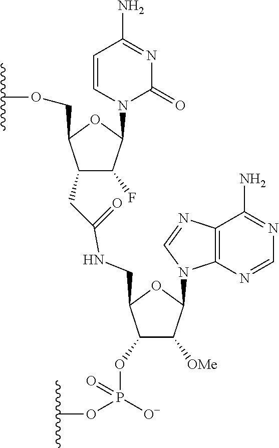 [SMILES: *OC[C@H]1O[C@@H](n2ccc(N)nc2=O)[C@H](F)[C@@H]1CC(=O)NC[C@H]1O[C@@H](n2cnc3c(N)ncnc32)[C@H](OC)[C@@H]1OP(=O)([O-])O*]

### Synthesis of NB-162: (2R,3R,4R,5R)-2-((2-((2S,3R,4R,5R)-5-(4-benzamido-2-oxopyrimidin-1(2H)-yl)-2-((bis(4-methoxyphenyl)(phenyl)methoxy)methyl)-4-fluorotetrahydrofuran-3-yl)acetamido)methyl)-5-(6-benzamido-9H-purin-9-yl)-4-methoxytetra hydrofuran-3-yl(2-cyanoethyl) diisopropylphosphoramidite

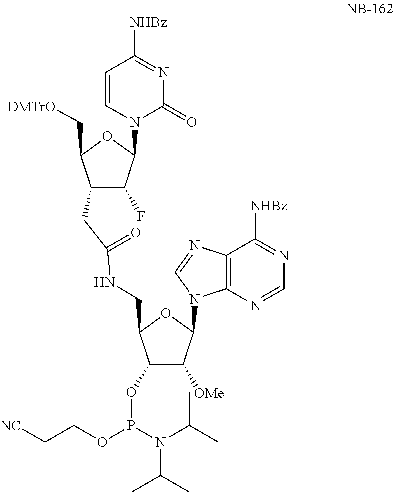

Dinucleotide NB-162 has been synthesized following the procedure described for compound NB-131 (example 21). Mass, 1282 [M+Na]⁺, ³¹P NMR (202 MHz, DMSO-d₆) δ 150.42, 149.60.

Oligonucleotide Comprising Dinucleotide NB-162 has been Synthesized Using the General Procedure Described in Example 1.

### Example 53: NB-163

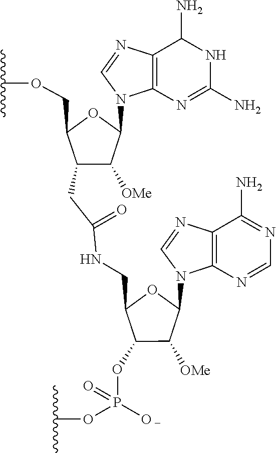 [SMILES: *OC[C@H]1O[C@@H](n2cnc3c2N=C(N)NC3N)[C@H](OC)[C@@H]1CC(=O)NC[C@H]1O[C@@H](n2cnc3c(N)ncnc32)[C@H](OC)[C@@H]1OP(=O)(O)O*]

### Synthesis of NB-163: (2R,3R,4R,5R)-5-(6-benzamido-9H-purin-9-yl)-2-((2-((2S,3R,4R,5R)-2-((bis(4-methoxyphenyl) (phenyl)methoxy)methyl)-5-(2-isobutyramido-6-oxo-1,6-dihydro-9H-purin-9-yl)-4-methoxytetra hydrofuran-3-yl)acetamido)methyl)-4-methoxy tetrahydrofuran-3-yl(2-cyanoethyl) diisopropylphosphoramidite

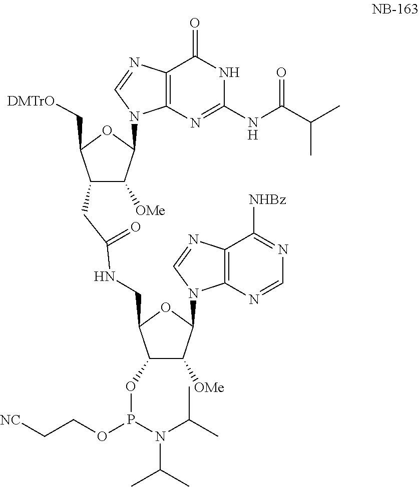

Dinucleotide NB-163 has been synthesized following the procedure described for compound NB-131 (example 21). Mass, 1278.4 [M+Na]⁺, ³¹P NMR (202 MHz, DMSO-d₆) δ 150.33, 149.66.

Oligonucleotide Comprising Dinucleotide NB-163 has been Synthesized Using the General Procedure Described in Example 1.

### Example 54: NB-164

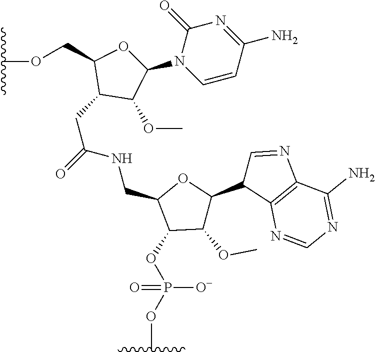 [SMILES: *OC[C@H]1O[C@@H](n2ccc(N)nc2=O)[C@H](OC)[C@@H]1CC(=O)NC[C@H]1O[C@@H](C2C=Nc3c(N)ncnc32)[C@H](OC)[C@@H]1OP(=O)([O-])O*]

### Synthesis of NB-164: (2R,3R,4R,5R)-2-((2-((2S,3R,4R,5R)-5-(4-benzamido-2-oxopyrimidin-1(2H)-yl)-2-((bis(4-methoxyphenyl)(phenyl)methoxy)methyl)-4-methoxytetrahydrofuran-3-yl)acetamido) methyl)-5-(6-benzamido-9H-purin-9-yl)-4-methoxytetrahydrofuran-3-yl(2-cyanoethyl) diisopropylphosphoramidite

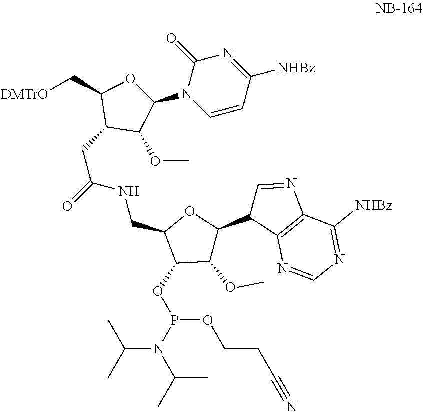

Dinucleotide NB-164 has been synthesized following the procedure described for compound NB-131 (example 21). Mass, 1293.8 [M+Na]⁺, ³¹P NMR (202 MHz, DMSO-d₆) δ 150.33, 149.65.

Oligonucleotide Comprising Dinucleotide NB-164 has been Synthesized Using the General Procedure Described in Example 1.

### Example 55: NB-165

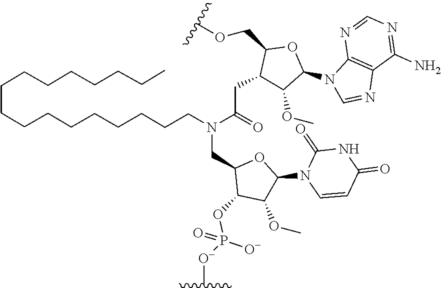

### Synthesis of NB-165: (2R,3R,4R,5R)-2-((2-((2S,3R,4R,5R)-5-(6-benzamido-9H-purin-9-yl)-2-((bis(4-methoxyphenyl)(phenyl)methoxy)methyl)-4-methoxytetrahydrofuran-3-yl)-N-heptadecylacetamido)methyl)-5-(2,4-dioxo-3,4-dihydropyrimidin-1(2H)-yl)-4-methoxytetrahydrofuran-3-yl(2-cyanoethyl) diisopropylphosphoramidite

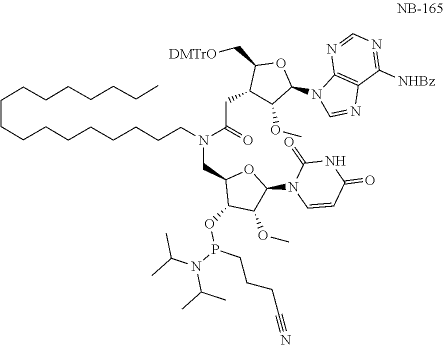

Dinucleotide NB-165 has been synthesized following the procedure described for compound NB-130 (example 20). Mass, 1429.1 [M+Na]⁺, ³¹P NMR (202 MHz, DMSO-d₆) δ 149.82, 149.71, 149.48, 149.28.

Oligonucleotide Comprising Dinucleotide NB-165 has been Synthesized Using the General Procedure Described in Example 1.

### Example 56: NB-166

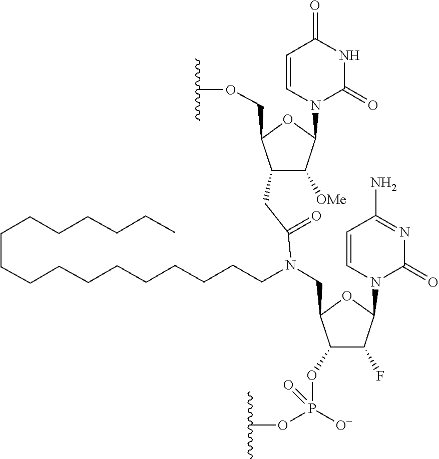 [SMILES: *OC[C@H]1O[C@@H](n2ccc(=O)[nH]c2=O)[C@H](OC)[C@@H]1CC(=O)N(CCCCCCCCCCCCCCCCC)C[C@H]1O[C@@H](n2ccc(N)nc2=O)[C@H](F)[C@@H]1OP(=O)([O-])O*]

### Synthesis of NB-166: (2R,3R,4R,5R)-5-(4-benzamido-2-oxopyrimidin-1(2H)-yl)-2-((2-((2S,3R,4R,5R)-2-((bis(4-methoxyphenyl)(phenyl)methoxy) methyl)-5-(2,4-dioxo-3,4-dihydropyrimidin-1(2H)-yl)-4-methoxytetrahydrofuran-3-yl)-N-heptadecylacetamido)methyl)-4-fluorotetrahydrofuran-3-yl(2-cyanoethyl) diisopropylphosphoramidite

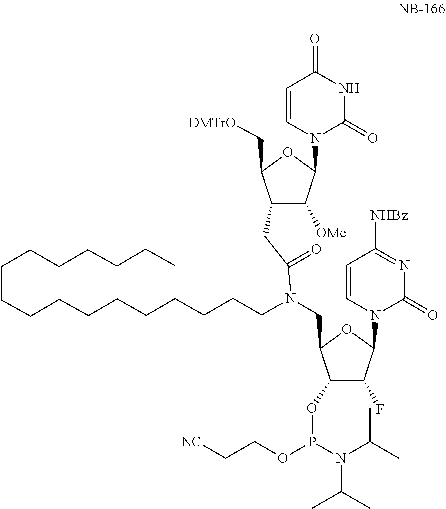

Dinucleotide NB-166 has been synthesized following the procedure described for compound NB-130 (example 20). Mass, 1393.6 [M+Na]⁺, ³¹P NMR (202 MHz, DMSO-d₆) δ 150.69, 150.66, 150.30, 150.27, 149.90, 149.88.

Oligonucleotide Comprising Dinucleotide NB-166 has been Synthesized Using the General Procedure Described in Example 1.

### Example 57: NB-167

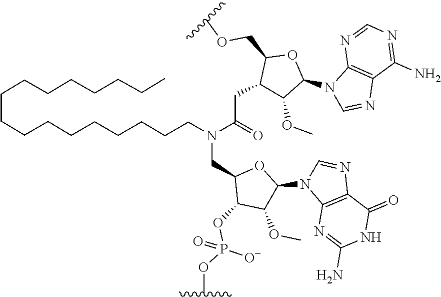 [SMILES: *OC[C@H]1O[C@@H](n2cnc3c(N)ncnc32)[C@H](OC)[C@@H]1CC(=O)N(CCCCCCCCCCCCCCCCC)C[C@H]1O[C@@H](n2cnc3c(=O)[nH]c(N)nc32)[C@H](OC)[C@@H]1OP(=O)([O-])O*]

### Synthesis of NB-167

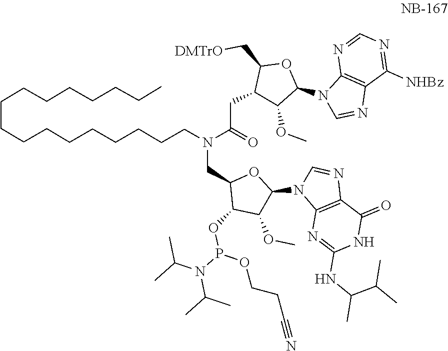

Dinucleotide NB-167 has been synthesized following the procedure described for compound NB-130 (example 20). ³¹P NMR (202 MHz, DMSO-d₆) δ 150.45, 150.40, 150.20, 149.98.

Oligonucleotide Comprising Dinucleotide NB-167 has been Synthesized Using the General Procedure Described in Example 1.

### Example 58: NB-168

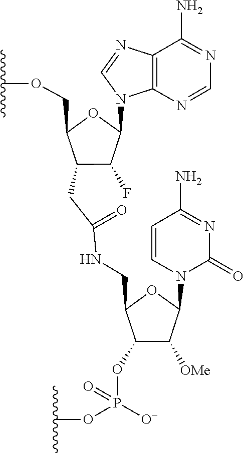 [SMILES: *OC[C@H]1O[C@@H](n2cnc3c(N)ncnc32)[C@H](F)[C@@H]1CC(=O)NC[C@H]1O[C@@H](n2ccc(N)nc2=O)[C@H](OC)[C@@H]1OP(=O)([O-])O*]

### Synthesis of NB-168:_(2R,3R,4R,5R)-5-(4-benzamido-2-oxopyrimidin-1(2H)-yl)-2-((2-((2S,3R,4R,5R)-5-(6-benzamido-9H-purin-9-yl)-2-((bis(4-methoxyphenyl)(phenyl) methoxy)methyl)-4-fluorotetrahydrofuran-3-yl)acetamido)methyl)-4-methoxytetrahydrofuran-3-yl(2-cyanoethyl)_diisopropylphosphoramidite

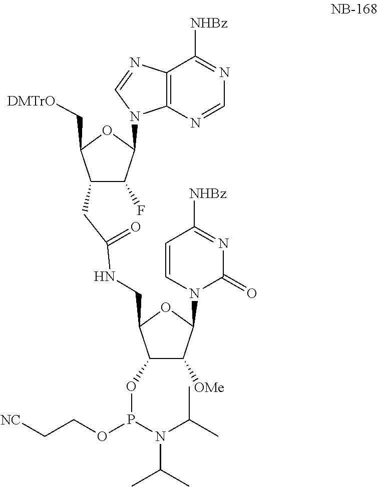

Dinucleotide NB-168 has been synthesized following the procedure described for compound NB-131 (example 21). Mass, 1281.8 [M+Na]⁺, ³¹P NMR (202 MHz, DMSO-d₆) δ 150.04, 149.73.

Oligonucleotide Comprising Dinucleotide NB-168 has been Synthesized Using the General Procedure Described in Example 1.

### Example 59: NB-169

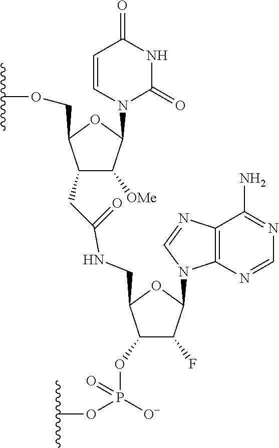 [SMILES: *OC[C@H]1O[C@@H](n2ccc(=O)[nH]c2=O)[C@H](OC)[C@@H]1CC(=O)NC[C@H]1O[C@@H](n2cnc3c(N)ncnc32)[C@H](F)[C@@H]1OP(=O)([O-])O*]

### Synthesis of NB-169: (2R,3R,4R,5R)-5-(6-benzamido-9H-purin-9-yl)-2-((2-((2S,3R,4R,5R)-2-((bis(4-methoxyphenyl) (phenyl)methoxy)methyl)-5-(2,4-dioxo-3,4-dihydropyrimidin-1(2H)-yl)-4-methoxytetra hydrofuran-3-yl)acetamido)methyl)-4-fluoro tetrahydrofuran-3-yl(2-cyanoethyl) diisopropylphosphoramidite

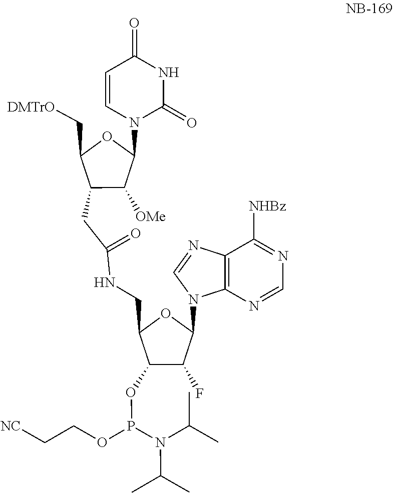

Dinucleotide NB-169 has been synthesized following the procedure described for compound NB-131 (example 21). Mass, 1179.2 [M+Na]⁺, ³¹P NMR (202 MHz, DMSO-d₆) δ 150.56, 150.54, 150.03, 149.97.

Oligonucleotide Comprising Dinucleotide NB-169 has been Synthesized Using the General Procedure Described in Example 1.

### Example 60: NB-170

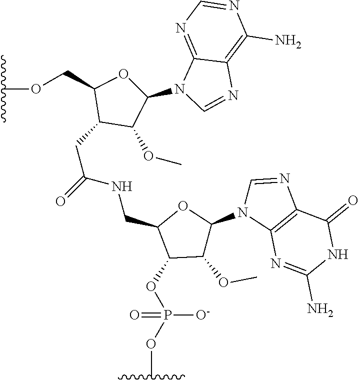 [SMILES: *OC[C@H]1O[C@@H](n2cnc3c(N)ncnc32)[C@H](OC)[C@@H]1CC(=O)NC[C@H]1O[C@@H](n2cnc3c(=O)[nH]c(N)nc32)[C@H](OC)[C@@H]1OP(=O)([O-])O*]

### Synthesis of NB-170: (2R,3R,4R,5R)-2-((2-((2S,3R,4R,5R)-5-(6-benzamido-9H-purin-9-yl)-2-((bis(4-methoxy phenyl)(phenyl)methoxy)methyl)-4-methoxy tetrahydrofuran-3-yl)acetamido)methyl)-5-(2-isobutyramido-6-oxo-1,6-dihydro-9H-purin-9-yl)-4-methoxytetrahydrofuran-3-yl(2-cyanoethyl) diisopropylphosphoramidite

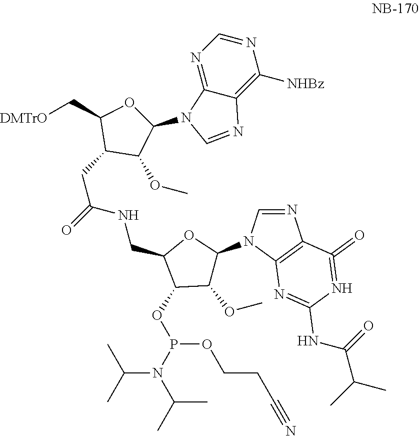

Dinucleotide NB-170 has been synthesized following the procedure described for compound NB-131 (example 21). Mass, 1300.04 [M+Na]⁺, ³¹P NMR (202 MHz, DMSO-d₆) δ 150.42, 150.15.

Oligonucleotide Comprising Dinucleotide NB-168 has been Synthesized Using the General Procedure Described in Example 1.

### Example 61: NB-171

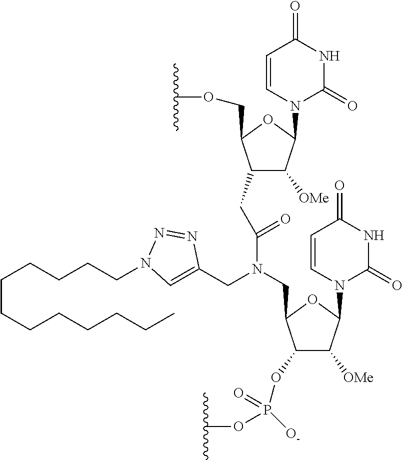

### Synthesis of NB-171

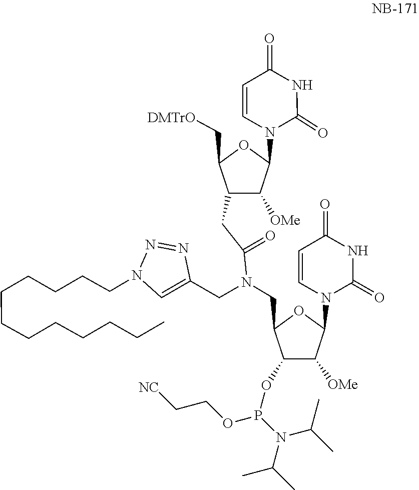

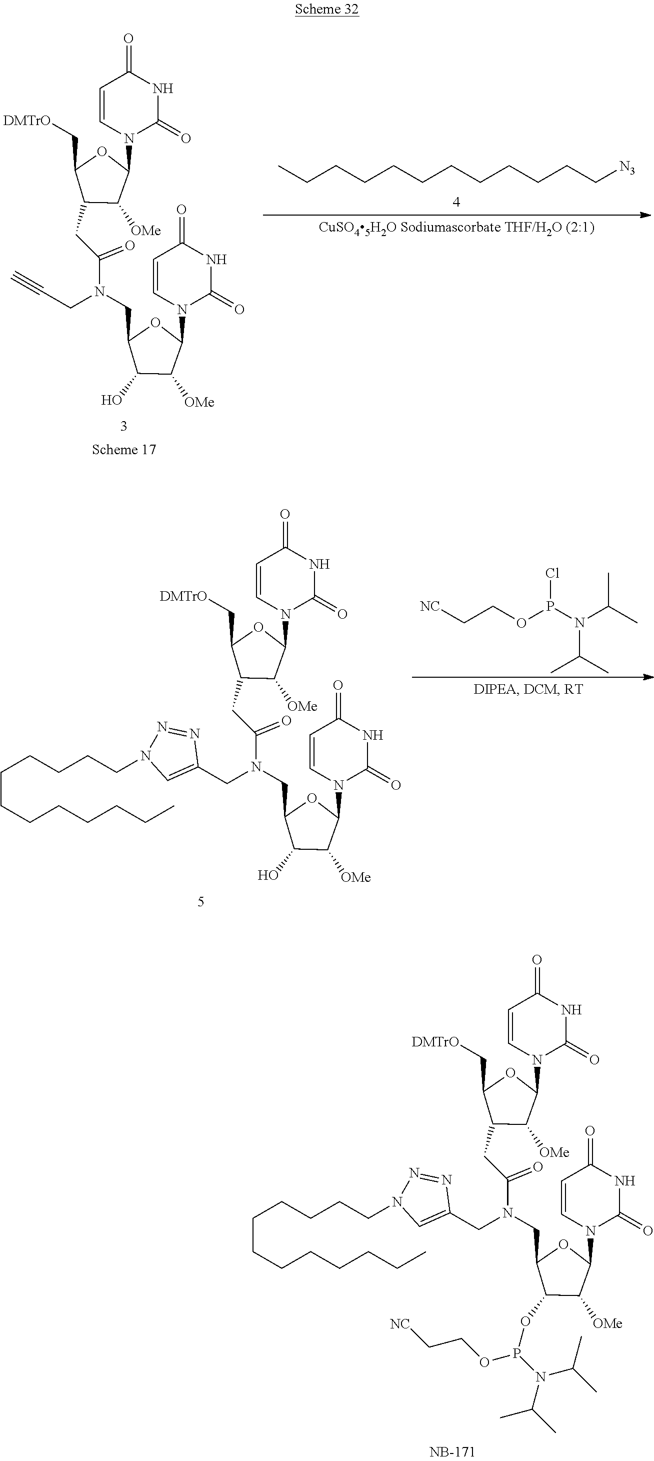

To a solution of alkyne 3 (1 g, 1.138 mmol, 1 eq) and azide 4 (288 mg, 1.365 mmol, 1 eq) in THF (10 mL) at RT was added a solution of CuSO₄˜5H₂O (142 mg, 0.114 mmol, 0.5 eq) in water (1 mL) followed by a solution of sodium ascorbate (170 mg, 0.8 mmol, 0.75 eq) in water (1 mL). The reaction was stirred for 12 hrs at RT. Then the reaction was diluted with EtOAc 50 mL and washed with NaHCO₃20 mL), dried over Na₂SO₄, filtered, and concentrated. The residue was purified by 0-5% EtOAc/MeOH to give 0.860 g of triazole (69%) as a beige solid, mass m/z=1114 (M+Na).

To a stirred solution of alcohol 5 (0.84 g, 0.77 mmol, 1 eq) and diisopropylethylamine (0.8 mL, 4.62 mmol, 6 eq), in DCM (10 mL), was added N, N-diisopropyl chlorophosphoramidite (0.51 mL 2.3 mmol, 3 eq) dropwise. The reaction mixture was stirred at room temperature for 1 h. Reaction mixture was quenched with Aq. Saturated NaHCO₃ solution (50 mL), extracted with DCM (100) ml, washed with brine (50 mL), dried over Na₂SO₄, and evaporated the crude product was loaded on to (pre-equilibrated with 1% Et₃N-EtOAc) Biotage silica gel column (25 g 20 μm), and purified by flash chromatography using MeOH/EtOAc 0-0% 1 CV, 0-3% 3-3% 10 CV, 3-5% 2.3 CV, 5% 1.4 CV, 5-10% 4.5 CV, 10%5 CV containing 1% Et₃N as an additive, 70% pure fractions and 30% of mixture with reagent. The mixture was concentrated and re dissolved in DCM 50 mL then 20 ml of Hexane was added slowly to get the precipitation, then the hexane was decanted, and repeated this process another time then the precipitate was dissolved in DCM and combined with the pure fractions, concentrated, and dried under high vacuum to obtain Phosphoramidite NB-171 (880 mg, 88%) as a white solid. 96% purity by HPLC and 90% purity by P31, Mass (m/z 1313 (M++Na). ³¹P NMR (202, MHz, DMSO-d6) δ 149.87, 149.68, 149.57.

Oligonucleotide Comprising Dinucleotide NB-171 has been Synthesized Using the General Procedure Described in Example 1.

### Example 62: NB-172

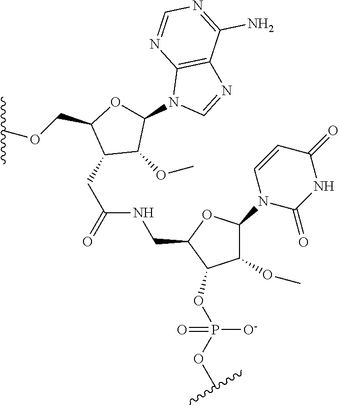 [SMILES: *OC[C@H]1O[C@@H](n2cnc3c(N)ncnc32)[C@H](OC)[C@@H]1CC(=O)NC[C@H]1O[C@@H](n2ccc(=O)[nH]c2=O)[C@H](OC)[C@@H]1OP(=O)([O-])O*]

### Synthesis of NB-172: (2R,3R,4R,5R)-2-((2-((2S,3R,4R,5R)-5-(6-benzamido-9H-purin-9-yl)-2-((bis(4-methoxy phenyl)(phenyl)methoxy)methyl)-4-methoxy tetrahydrofuran-3yl)acetamido)methyl)-5-(2,4-dioxo-3,4-dihydropyrimidin-1(2H)-yl)-4-methoxy tetrahydrofuran-3-yl(2-cyanoethyl) diisopropylphosphoramidite

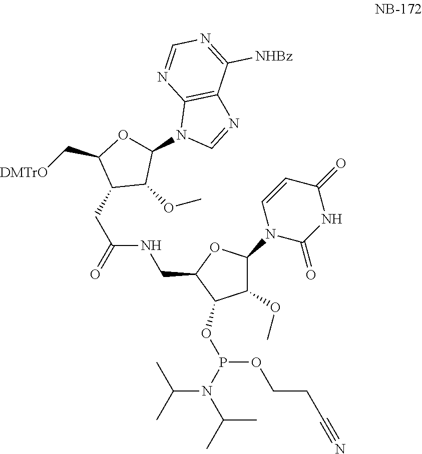

Dinucleotide NB-172 has been synthesized following the procedure described for compound NB-131 (example 21). Mass, 1191.1 [M+Na]⁺, ³¹P NMR (202 MHz, DMSO-d₆) δ 150.04, 149.61.

Oligonucleotide Comprising Dinucleotide NB-172 has been Synthesized Using the General Procedure Described in Example 1.

### Example 63: NB-173

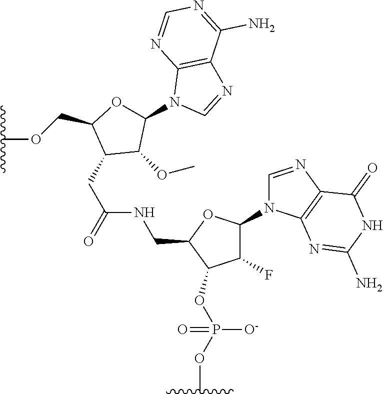 [SMILES: *OC[C@H]1O[C@@H](n2cnc3c(N)ncnc32)[C@H](OC)[C@@H]1CC(=O)NC[C@H]1O[C@@H](n2cnc3c(=O)[nH]c(N)nc32)[C@H](F)[C@@H]1OP(=O)([O-])O*]

### Synthesis of NB-173: (2R,3R,4R,5R)-2-((2-((2S,3R,4R,5R)-5-(6-benzamido-9H-purin-9-yl)-2-((bis(4-methoxy phenyl)(phenyl)methoxy)methyl)-4-methoxy tetrahydrofuran-3-yl)acetamido)methyl)-4-fluoro-5-(2-isobutyramido-6-oxo-1,6-dihydro-9H-purin-9-yl)tetrahydrofuran-3-yl(2-cyanoethyl) diisopropylphosphoramidite

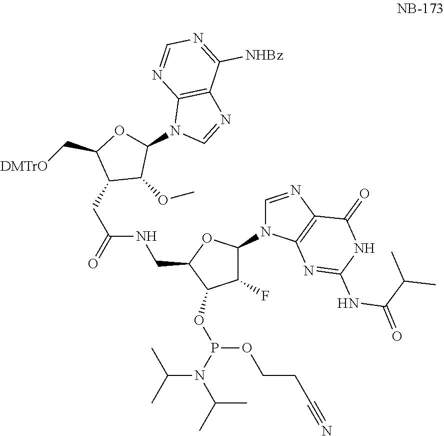

Dinucleotide NB-173 has been synthesized following the procedure described for compound NB-131 (example 21). Mass, 1288.3 [M+Na]⁺, ³¹P NMR (202 MHz, DMSO-d₆) δ 150.68, 150.62, 150.37, 150.34.

Oligonucleotide Comprising Dinucleotide NB-173 has been Synthesized Using the General Procedure Described in Example 1.

### Example 64: NB-174

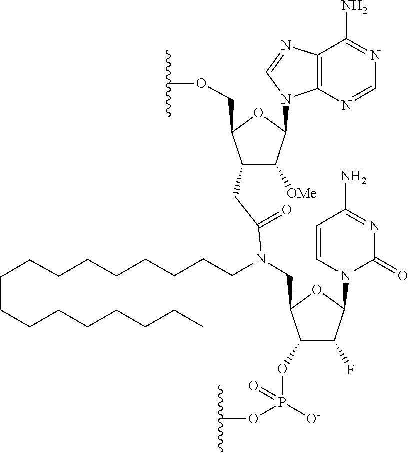 [SMILES: *OC[C@H]1O[C@@H](n2cnc3c(N)ncnc32)[C@H](OC)[C@@H]1CC(=O)N(CCCCCCCCCCCCCCCCC)C[C@H]1O[C@@H](n2ccc(N)nc2=O)[C@H](F)[C@@H]1OP(=O)([O-])O*]

### Synthesis of NB-174

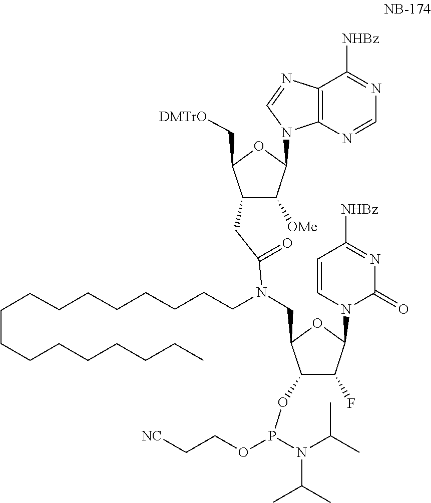

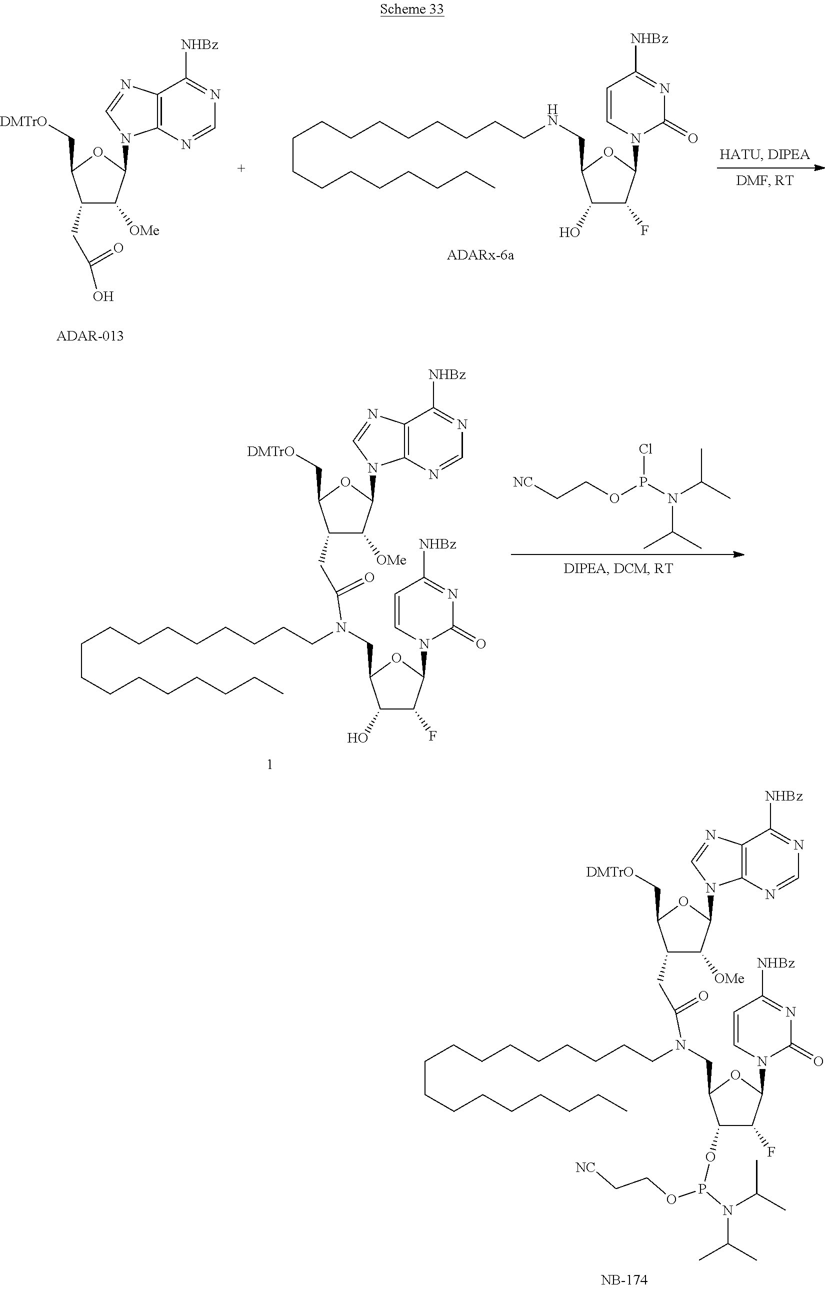

Add DMF (34 mL) to a flask with ADAR-013 (5.0 g, 6.85 mmol, 1 equiv) and amine ADARx-6a (4.42 g, 7.54 mmol, 1.1 equiv) and HATU (3.91 g, 10.3 mmol, 1.5 equiv), dissolves over 5 min. Add DIPEA (3.6 mL, 21 mmol, 3.0 equiv) dropwise over 45 seconds. After 90 min, the reaction mixture was added dropwise to 50% sat'd NaCl (200 mL). The resulting solids were collected by filtration with vacuum, then rinsed with water (50 mL). After drying with vacuum overnight, the resulting solids were dissolved in DCM (200 mL) and washed with sat'd NaCl (50 mL). The aqueous layer was extracted with DCM (100 mL). The combined organic extracts were dried with anhydrous sodium sulfate, were filtered through Celite, and were concentrated under reduced pressure. The resulting residue was purified by FCC on silica gel (100 g HC, (1) 50% EtOAc/Hexanes, [2 CV], (2) 0-20% MeOH/EtOAc; 0%[1, f3-8], 0-5%[5, A9-B9], 5% [1, B10-15], 5-20%[4, fB16-C11]). Pure fractions A26-B20 were collected to afford amide (3.84 g, 43% yield) as a white solid and mixed fractions B21-C3 afforded amide (3.43 g). Mixed fractions were repurified by FCC on silica gel (50 g HC, 0-60% acetone/DCM; 0-10%[1], 10-24%[3.3], 24%[6.5, f1-18], 25-40%[5, f19-B5], 40%[1, fB5-9], 40-60%[3, B10-19], 60%[3, B20-28]). Collected fA28-B28 to afford amide (2.82 g, 31% yield—single peak by LCMS) for combined 6.66 g (75% yield) of amide 1.

Evacuate and backfill a flask containing alcohol 1 (2.82 g, 2.17 mmol, 1 equiv). Then, dissolve in DCM (11 mL), add DIPEA (1.52 mL, 8.7 mmol, 4.0 equiv), cool to 0° C., and then add CE-DIP-Cl (1.21 mL, 5.4 mmol, 2.5 equiv) then stir at RT. After 2 h, additional CE-DIP-Cl (0.24 mL, 1.1 mmol, 0.5 equiv) was added. After an additional 2 h, add additional DIPEA (0.38 mL, 2.2 mmol, 1.0 equiv) and CE-DIP-Cl (0.24 mL, 1.1 mmol, 0.5 equiv). After 30 min, add methanol (2 mL) and stir for 15 min before transferring to smaller flask with DCM and concentrating. The reaction mixture was purified by FCC on silica gel (100 g HC, 0-35% EtOAc-EtOH (3:1)/Heptane+1% NEt3 additive; 0%[1], 0-35%[10 CV, f1-7 only 220 nM observed; f9 @27%; 254 nM only f9-21]). Based on TLC and LCMS collect f10-19, concentrate under reduced pressure. The resulting residue was dissolved in DCM and triturated with hexanes (not lots of solids), concentrated, redissolved in DCM and triturated with hexanes, diluted further with Et₂O and partially concentrated until solids precipitated, diluted further with hexanes, and then the supernatant was decanted (lots of 219 peak observed). The resulting solids were dissolved in DCM and concentrated under reduced pressure to afford amidite NB-174 (2.334 g) as a faint yellow foam. Mass observed M+Na=1520.1. NMR shows ˜20% reagent remaining. Redissolve in EtOAc and triturate with hexanes, decant supernatant, repeat 2×. HPLC purity 97%. Net 2.123 g (65%). ³¹P NMR (202, MHz, DMSO-d6) δ 150.73, 150.70, 150.55, 150.50, 150.35, 150.33, 149.83, 149.81.

Oligonucleotide Comprising Dinucleotide NB-174 has been Synthesized Using the General Procedure Described in Example 1.

### Example 65: NB-175

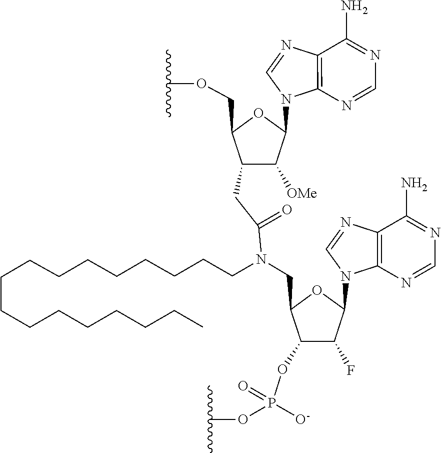 [SMILES: *OC[C@H]1O[C@@H](n2cnc3c(N)ncnc32)[C@H](OC)[C@@H]1CC(=O)N(CCCCCCCCCCCCCCCCC)C[C@H]1O[C@@H](n2cnc3c(N)ncnc32)[C@H](F)[C@@H]1OP(=O)([O-])O*]

### Synthesis of NB-175

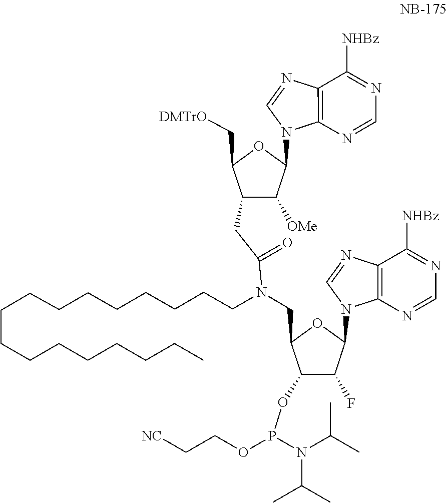

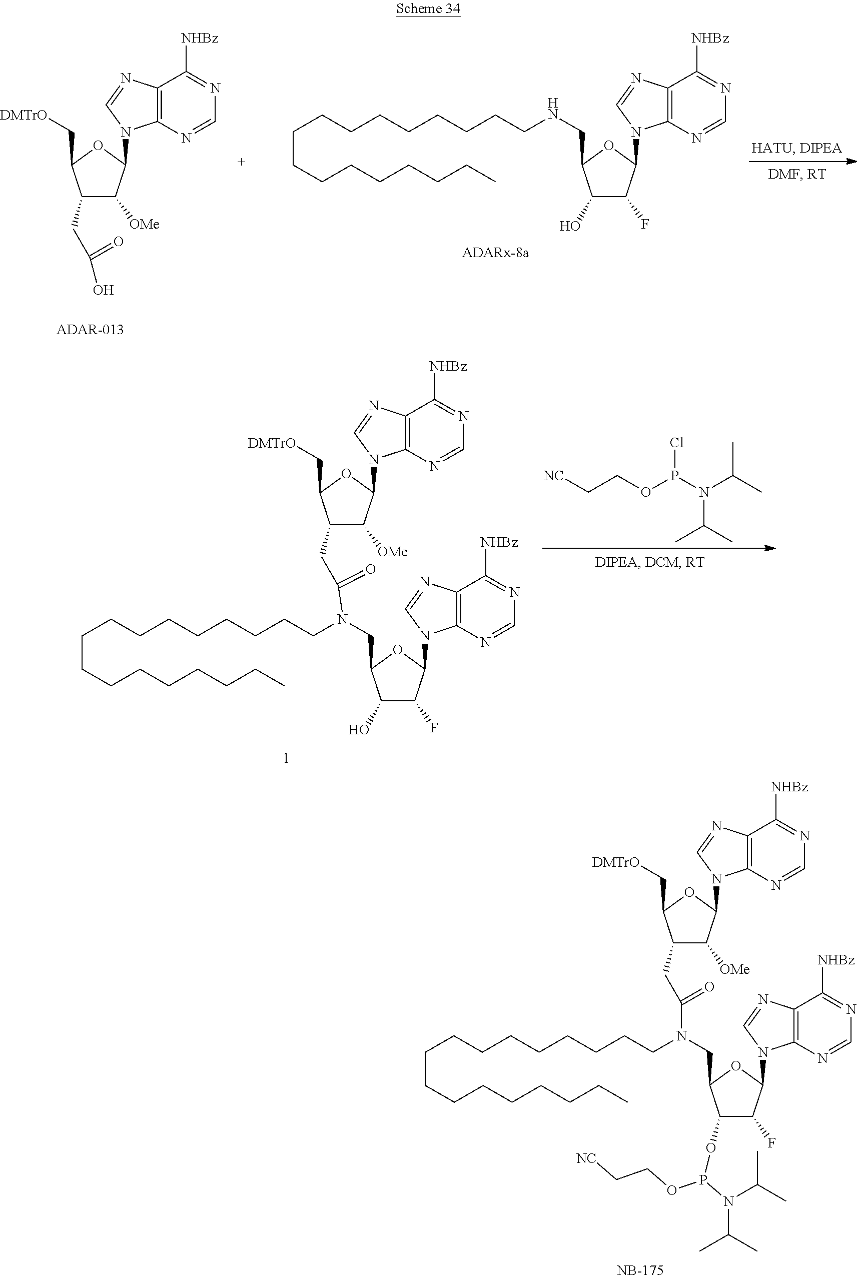

Add DMF (22 mL) to acid ADAR-013 (4.00 g, 5.5 mmol, 1 equiv), lipid amine ADARx-8a (3.68 g, 6.03 mmol, 1.1 equiv), and HATU (3.13 g, 8.22 mmol, 1.5 equiv). Sonicate for 1 min to dissolve >95%. Then, add DIPEA (2.87 mL, 16.4 mmol, 3.0 equiv) dropwise. After 2.5 h, the reaction mixture was added dropwise to 50% sat'd NaCl (500 mL). The resulting solids were collected by filtration and were rinsed with H₂O (50 mL). The resulting crude solids (9.7 g) were redissolved in DCM (˜200 mL) and dried with anhydrous sodium sulfate, filtered through Celite, and concentrated under reduced pressure. The resulting crude residue was purified by FCC on silica gel (100 g HC, 0-60% acetone/DCM; 0%[1], 0-10%[0.5], 10-15%[1], 15%[2], 15-30%[3], 30%[4, f2-21], 30□40%[2, f22-B5], 40%[5, B6-C5], 40-60%[2, C6-28]). Based on TLC and LCMS, collected fB2-C14 to afford amide (4.25 g, 59% yield) as a white solid. Single peak by LCMS.

Sparge a solution of alcohol 1 (4.25 g, 3.213 mmol). +DIPEA (2.245 mL, 12.854 mmol) in DCM (16 mL) at 0 for 5 min, add CE-DIP-Cl, (1.78 mL, 8.033 mmol) then stir at RT. After 3 h, add MeOH (2 mL), partially concentrate, then purify by FCC on silica gel (25-100% EtOAc/Heptane+1% NEt₃ [4, CV]. Collect f1-6 and f7-15 separately, concentrate, triturate with hexanes from DCM (1×) obtained 4.53 g (92%) of NB-175. Mass (m/z 523 (M++1). ³¹P NMR (202, MHz, DMSO-d6) δ 150.25, 150.22, 150.04, 150.00, 149.93, 149.86, 149.69, 149.67.

Oligonucleotide Comprising Dinucleotide NB-175 has been Synthesized Using the General Procedure Described in Example 1.

### Example 66: NB-176

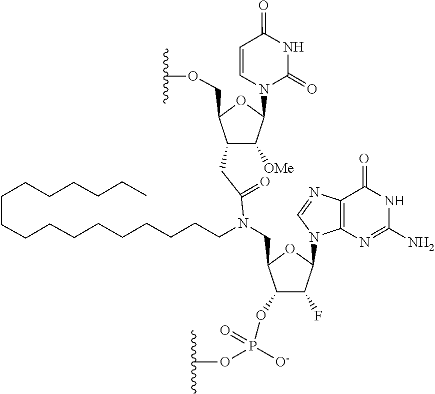 [SMILES: *OC[C@H]1O[C@@H](n2ccc(=O)[nH]c2=O)[C@H](OC)[C@@H]1CC(=O)N(CCCCCCCCCCCCCCCCC)C[C@H]1O[C@@H](n2cnc3c(=O)[nH]c(N)nc32)[C@H](F)[C@@H]1OP(=O)([O-])O*]

### Synthesis of NB-176: (2R,3R,4R,5R)-2-((2-((2S,3R,4R,5R)-2-((bis(4-methoxyphenyl)(phenyl)methoxy) methyl)-5-(2,4-dioxo-3,4-dihydropyrimidin-1(2H)-yl)-4-methoxytetrahydrofuran-3-yl)-N-heptadecylacetamido)methyl)-4-fluoro-5-(2-isobutyramido-6-oxo-1,6-dihydro-9H-purin-9-yl)tetrahydrofuran-3-yl (2-cyanoethyl) diisopropylphosphoramidite

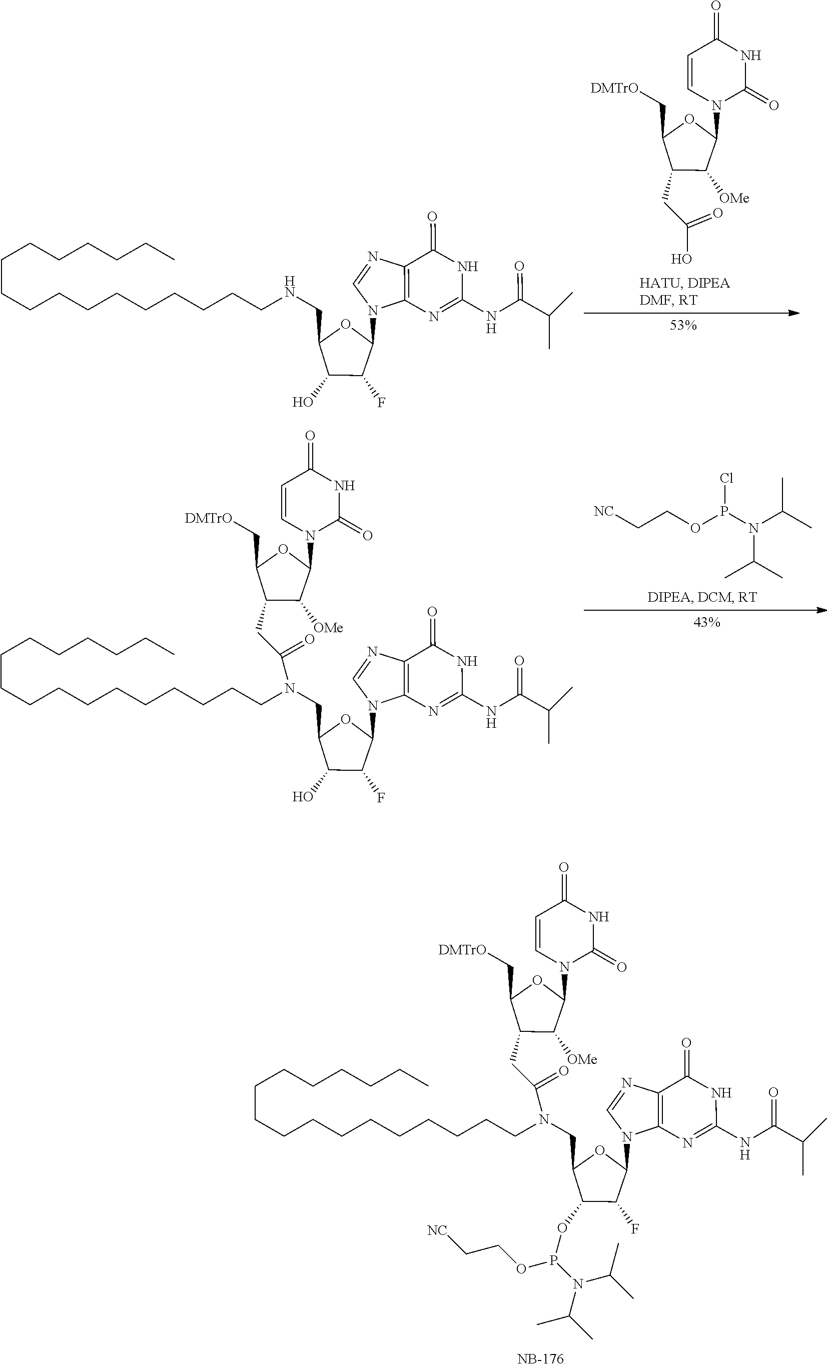

### Step 1: N-(9-((2R,3R,4R,5R)-5-((2-((2S,3R,4R,5R)-2-((bis(4-methoxyphenyl)(phenyl)methoxy) methyl)-5-(2,4-dioxo-3,4-dihydropyrimidin-1(2H)-yl)-4-methoxytetrahydrofuran-3-yl)-N-heptadecylacetamido)methyl)-3-fluoro-4-hydroxytetrahydrofuran-2-yl)-6-oxo-6,9-dihydro-1H-purin-2-yl)isobutyramide

To a solution of 2-((2S,3R,4R,5R)-2-((bis(4-methoxyphenyl)(phenyl)methoxy)methyl)-5-(2,4-dioxo-3,4-dihydropyrimidin-1(2H)-yl)-4-methoxytetrahydrofuran-3-yl)acetic acid (4 g, 6.64 mmol) in anhydrous DMF (30 ml), N-(9-((2R,3R,4R,5R)-3-fluoro-5-((heptadecylamino)methyl)-4-hydroxytetrahydrofuran-2-yl)-6-oxo-6,9-dihydro-1H-purin-2-yl)isobutyramide (4.32 g, 7.3 mmol), HATU (3.78 g, 9.95 mmol) and DIPEA (3.46 ml, 19.91 mmol) were added. The reaction mixture was stirred at room temperature under an inert atmosphere for 18 hours. Reaction mixture was diluted with water (150 ml). The resulting precipitates were filtered off and washed with water (50 ml). The solids were dried under vacuum, redissolved in DCM, and purified by silica gel column chromatography using a gradient 0-5% MeOH in EtOAc to afford the titled compound (4.15 g, 53%) as an off-white solid. MS (ESI) m/z 1177.6 [M+1]⁺.

### Step 2: (2R,3R,4R,5R)-2-((2-((2S,3R,4R,5R)-2-((bis(4-methoxyphenyl)(phenyl)methoxy) methyl)-5-(2,4-dioxo-3,4-dihydropyrimidin-1(2H)-yl)-4-methoxytetrahydrofuran-3-yl)-N-heptadecylacetamido)methyl)-4-fluoro-5-(2-isobutyramido-6-oxo-1,6-dihydro-9H-purin-9-yl)tetrahydrofuran-3-yl (2-cyanoethyl) diisopropylphosphoramidite

To a stirred solution of N-(9-((2R,3R,4R,5R)-5-((2-((2S,3R,4R,5R)-2-((bis(4-methoxyphenyl)(phenyl)methoxy) methyl)-5-(2,4-dioxo-3,4-dihydropyrimidin-1(2H)-yl)-4-methoxytetrahydrofuran-3-yl)-N-heptadecylacetamido)methyl)-3-fluoro-4-hydroxytetrahydro furan-2-yl)-6-oxo-6,9-dihydro-1H-purin-2-yl)isobutyramide (2.1 g, 1.78 mmol) and DIPEA (1.86 ml, 10.70 mmol) in DCM (26 ml), N,N-diisopropyl chlorophosphoramidite (0.83 ml, 3.74 mmol) was added dropwise. The reaction mixture was stirred at room temperature under inert atmosphere for 16 hours. The reaction mixture was quenched with aq. saturated NaHCO₃ solution (40 ml) and extracted with DCM (3×50 ml). The combined organic extracts were washed with brine (25 ml), dried over anhydrous Na₂SO₄ and concentrated under reduced pressure. The crude product was purified by flash chromatography using 20-100% EtOAc/hexane and later 0-20% MeOH/EtOAc containing 1% Et₃N as an additive for all solvents. Pure fractions were combined and concentrated, dried under high vacuum to obtain the titled phosphoramidite NB-176 as a white solid (1.05 g, 43%). ³¹P NMR (202, MHz, DMSO-d₆) δ 150.79, 150.20.

Oligonucleotide Comprising Dinucleotide NB-176 has been Synthesized Using the General Procedure Described in Example 1.

### Example 67: NB-177

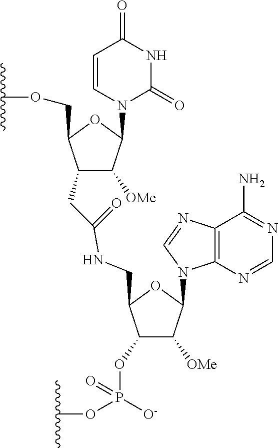 [SMILES: *OC[C@H]1O[C@@H](n2ccc(=O)[nH]c2=O)[C@H](OC)[C@@H]1CC(=O)NC[C@H]1O[C@@H](n2cnc3c(N)ncnc32)[C@H](OC)[C@@H]1OP(=O)([O-])O*]

### Synthesis of NB-177: (2R,3R,4R,5R)-5-(6-benzamido-9H-purin-9-yl)-2-((2-((2S,3R,4R,5R)-2-((bis(4-methoxyphenyl)(phenyl)methoxy)methyl)-5-(2,4-dioxo-3,4-dihydropyrimidin-1(2H)-yl)-4-methoxytetrahydrofuran-3-yl)acetamido)methyl)-4-methoxytetrahydrofuran-3-yl (2-cyanoethyl) diisopropylphosphoramidite

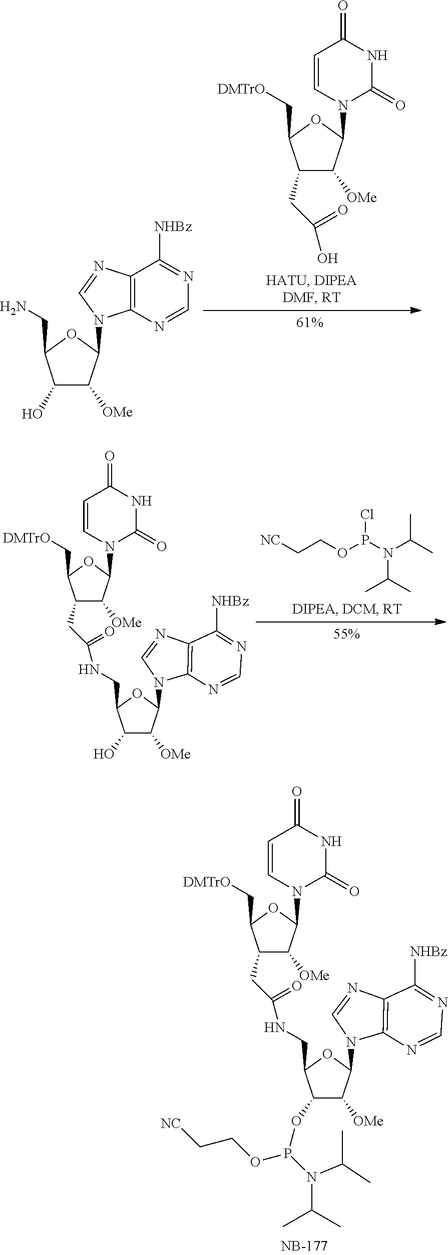

Step 1: N-(9-((2R,3R,4R,5R)-5-((2-((2S,3R,4R,5R)-2-((bis(4-methoxyphenyl)(phenyl)methoxy) methyl)-5-(2,4-dioxo-3,4-dihydropyrimidin-1(2H)-yl)-4-methoxytetrahydrofuran-3-yl)acetamido)methyl)-4-hydroxy-3-methoxytetrahydrofuran-2-yl)-9H-purin-6-yl)benzamide

To a solution of 2-((2S,3R,4R,5R)-2-((bis(4-methoxyphenyl)(phenyl)methoxy)methyl)-5-(2,4-dioxo-3,4-dihydropyrimidin-1(2H)-yl)-4-methoxytetrahydrofuran-3-yl)acetic acid (4 g, 6.64 mmol) in anhydrous DMF (30 ml), N-(9-((2R,3R,4R,5R)-5-(aminomethyl)-4-hydroxy-3-methoxytetrahydrofuran-2-yl)-9H-purin-6-yl)benzamide (2.81 g, 7.3 mmol), HATU (3.78 g, 9.95 mmol) and DIPEA (3.46 ml, 19.91 mmol) were added. The reaction mixture was stirred at room temperature under inert atmosphere for 15 hours. Reaction mixture was diluted with water (150 ml). Resulting precipitates were filtered off and washed with water (50 ml). The solids were dried under vacuum, redissolved in DCM, and purified by silica gel column chromatography using a gradient 0-5% MeOH in EtOAc to afford the titled compound (3.9 g, 61%) as an off-white solid. MS (ESI) m/z 969.9 [M+1]⁺.

### Step 2: (2R,3R,4R,5R)-5-(6-benzamido-9H-purin-9-yl)-2-((2-((2S,3R,4R,5R)-2-((bis(4-methoxyphenyl)(phenyl)methoxy)methyl)-5-(2,4-dioxo-3,4-dihydropyrimidin-1(2H)-yl)-4-methoxytetrahydrofuran-3-yl)acetamido)methyl)-4-methoxytetrahydrofuran-3-yl (2-cyanoethyl) diisopropylphosphoramidite

To a stirred solution of N-(9-((2R,3R,4R,5R)-5-((2-((2S,3R,4R,5R)-2-((bis(4-methoxyphenyl)(phenyl)methoxy)methyl)-5-(2,4-dioxo-3,4-dihydropyrimidin-1(2H)-yl)-4-methoxytetrahydrofuran-3-yl)acetamido)methyl)-4-hydroxy-3-methoxytetrahydrofuran-2-yl)-9H-purin-6-yl)benzamide (1.87 g, 1.93 mmol) and DIPEA (2 ml, 11.58 mmol) in DCM (25 ml), N,N-diisopropyl chlorophosphoramidite (1.29 ml, 5.79 mmol) was added dropwise. The reaction mixture was stirred at room temperature under inert atmosphere for 16 hours. Reaction mixture was quenched with aq. saturated NaHCO₃ solution (40 ml) and extracted with DCM (3×50 ml). The combined organic extracts were washed with brine (25 ml), dried over anhydrous Na₂SO₄ and concentrated under reduced pressure. The crude product was purified by flash chromatography using 20-100% EtOAc/hexane and later 0-20% MeOH/EtOAc containing 1% Et₃N as an additive for all solvents. Pure fractions were combined and concentrated, dried under high vacuum to obtain the titled phosphoramidite NB-177 as a white solid (1.25 g, 55%). ³¹P NMR (202, MHz, DMSO-d₆) δ 150.34, 149.68.

Oligonucleotide Comprising Dinucleotide NB-177 has been Synthesized Using the General Procedure Described in Example 1.

### Example 68: NB-178

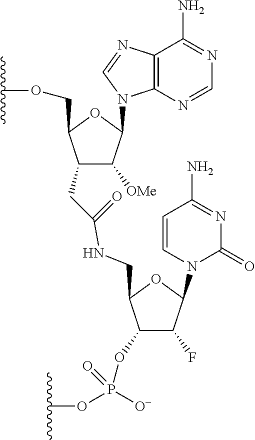 [SMILES: *OC[C@H]1O[C@@H](n2cnc3c(N)ncnc32)[C@H](OC)[C@@H]1CC(=O)NC[C@H]1O[C@@H](n2ccc(N)nc2=O)[C@H](F)[C@@H]1OP(=O)([O-])O*]

### Synthesis of NB-178

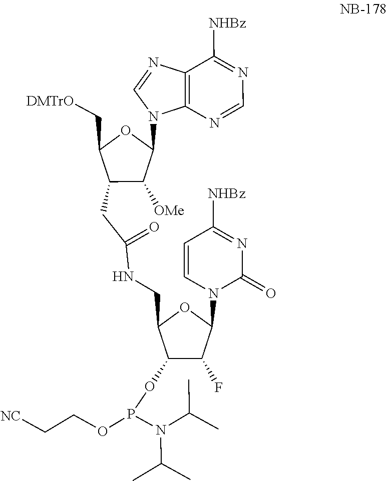

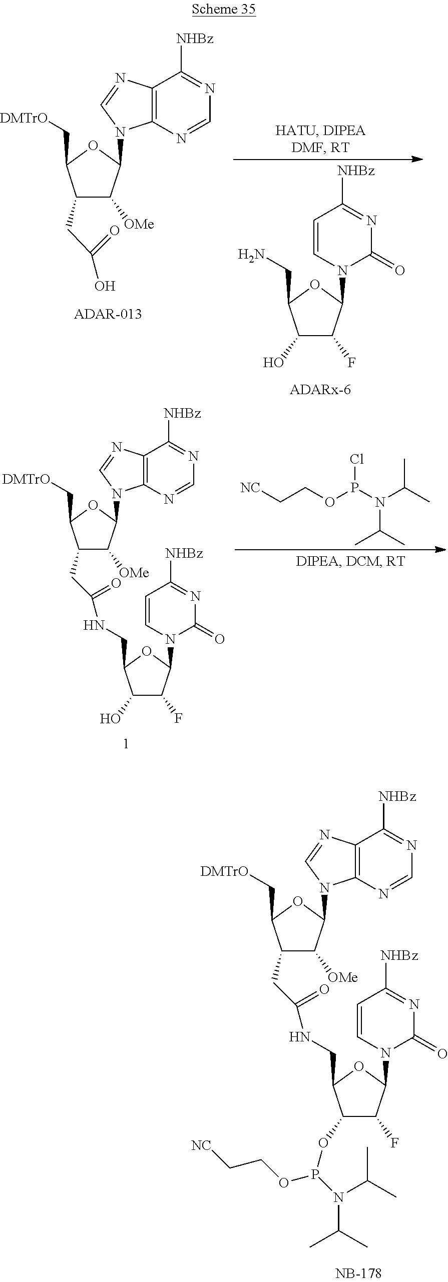

To a stirred solution of acid ADAR-013 (4 g, 5.487 mmol, 1 eq), amine ADARx-6 (2.1 g, 6.036 mmol, 1.1 eq) and HATU (3.12 g, 8.23 mmol, 1.5 eq) in DMF (30 mL) was added DIPEA (2.86 mL, 16.46 mmol, 3 eq) and the mixture was stirred for 3 h at room temperature, reaction mixture was added dropwise to a vigorously stirring water (250 ml), precipitated solids were filtered and washed with water, the solids were dried and re dissolved in DCM dried over Na₂SO₄ and concentrated, the residue was dissolved in EtOAc then purified by column chromatography using Ethyl acetate/MeOH, 0-10% as an eluent, pure fractions were combined and concentrated to obtain amide 1 (3.52 g, 55%) as a beige solid. LCMS m/z 1082 (M+Na).

To a solution of alcohol 1 (3.5 g, 3.302 mmol, 1 eq) and diisopropylethylamine (3.44 mL, 19.8 mmol, 6 eq), in DCM (30 mL), was added N, N-diisopropyl chlorophosphoramidite (2.2 mL, 9.9 mmol, 3 eq) dropwise, and the mixture was stirred at room temperature for 5 h. Reaction mixture was quenched with Aq. Saturated NaHCO₃ solution (100 mL), extracted with DCM (100) ml, washed with brine (100 mL), dried over Na₂SO₄, and evaporated the crude product was loaded on to (pre-equilibrated with 1% Et₃N-EtOAc) Biotage silica gel column (100 g 20 μm), and purified by flash chromatography using MeOH/EtOAc 0-10% 10 CV, 10% 5 CV, containing 1% Et₃N as an additive, pure fractions were combined and concentrated to obtain Phosphoramidite NB-178, 2.3 g, 55% as a beige solid. 90% purity by LCMS and P31, Mass (m/z 1282 (M++Na) H1-NMR and P31-NMR are corresponding with product. ³¹P NMR (202, MHz, DMSO-d6) δ 150.68, 150.66, 150.56, 150.50.

Oligonucleotide Comprising Dinucleotide NB-178 has been Synthesized Using the General Procedure Described in Example 1.

### Example 69: NB-179

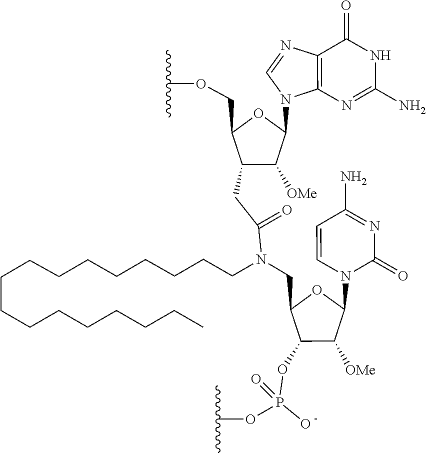 [SMILES: *OC[C@H]1O[C@@H](n2cnc3c(=O)[nH]c(N)nc32)[C@H](OC)[C@@H]1CC(=O)N(CCCCCCCCCCCCCCCCC)C[C@H]1O[C@@H](n2ccc(N)nc2=O)[C@H](OC)[C@@H]1OP(=O)(O)O*]

### Synthesis of NB-179

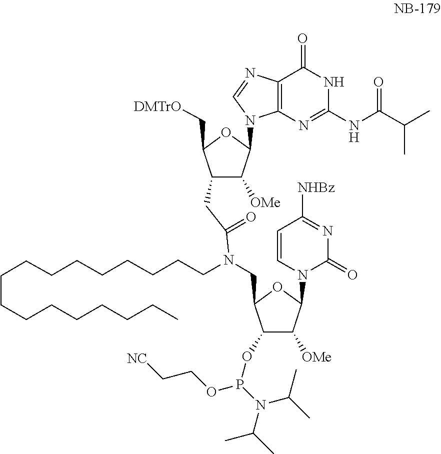

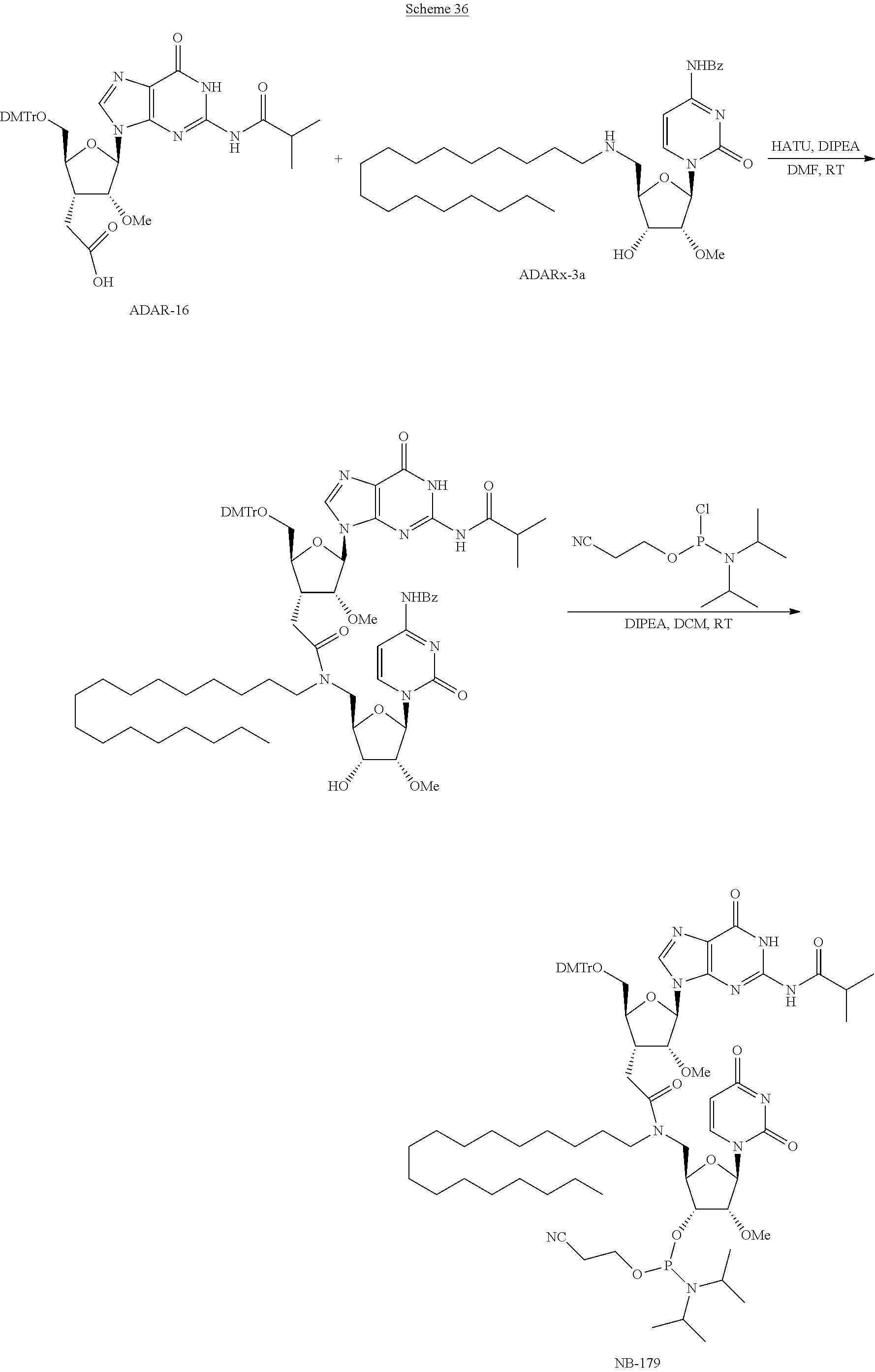

To a stirred solution of acid ADAR-16 (4 g, 5.626 mmol, 1 eq), amine ADARx-3a (3.95 g, 6.610 mmol, 1.1 eq) and HATU (3.2 g, 8.4 mmol, 1.5 eq) in DMF (30 mL) was added DIPEA (2.93 mL, 16.878 mmol, 3 eq) and the mixture was stirred for 3 h at room temperature, reaction mixture was added dropwise to a vigorously stirring water (250 ml), precipitated solids were filtered and washed with water, the solids were dried and re dissolved in DCM dried over Na₂SO₄ and concentrated, the residue was dissolved in EtOAc then purified by column chromatography using Ethyl acetate/MeOH, 0-5% as an eluent, pure fractions were combined and concentrated to obtain amide 1 (6 g, 70%) as a beige solid. LCMS m/z 1314 (M+Na).

To a stirred solution of alcohol 1 (6 g, 4.644 mmol, 1 eq) and diisopropylethylamine (4.8 mL, 27.86 mmol, 6 eq), in DCM (80 mL), was added N, N-diisopropyl chlorophosphoramidite (3.1 mL 14 mmol, 3 eq) dropwise. The reaction mixture was stirred at room temperature for 3 h. Reaction mixture was quenched with Aq. Saturated NaHCO₃ solution (100 mL), extracted with DCM (200) ml, washed with brine (200 mL), dried over Na₂SO₄, and evaporated the crude product was loaded on to (pre-equilibrated with 1% Et₃N-EtOAc) Biotage silica gel column (100 g 20 μm), and purified by flash chromatography using MeOH/EtOAc 2-10% 5 CV, 10% 5 CV, containing 1% Et₃N as an additive, gave 2 g of pure product and 3 g of mixture with reagent. The mixture was concentrated and re dissolved in DCM 50 mL then 30 ml of Hexane was added slowly to get the precipitation, then triturated and hexane was decanted, and repeated this process another time then the precipitate was dissolved in DCM and combined with 2 g of the pure compound, concentrated and dried under high vacuum to obtain Phosphoramidite NB-179, 4.1 g, 59% as an off white solid. 98% purity by, HPLC and 93% purity by P31, Mass (m/z 1514 (M++Na), ³¹P NMR (202, MHz, DMSO-d6) δ 150.00, 149.77, 149.20.

Oligonucleotide Comprising Dinucleotide NB-179 has been Synthesized Using the General Procedure Described in Example 1.

### Example 70: NB-180

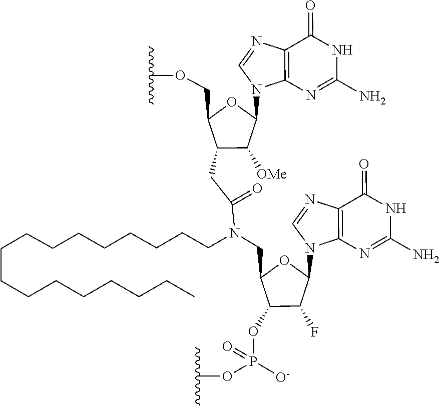 [SMILES: *OC[C@H]1O[C@@H](n2cnc3c(=O)[nH]c(N)nc32)[C@H](OC)[C@@H]1CC(=O)N(CCCCCCCCCCCCCCCCC)C[C@H]1O[C@@H](n2cnc3c(=O)[nH]c(N)nc32)[C@H](F)[C@@H]1OP(=O)([O-])O*]

### Synthesis of NB-180

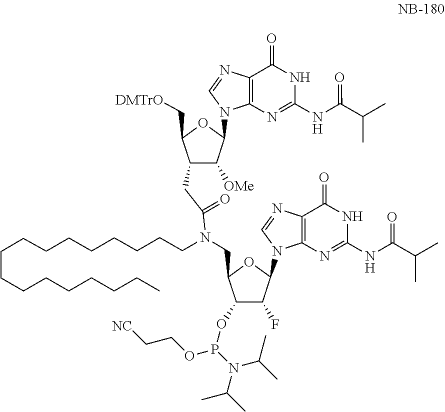

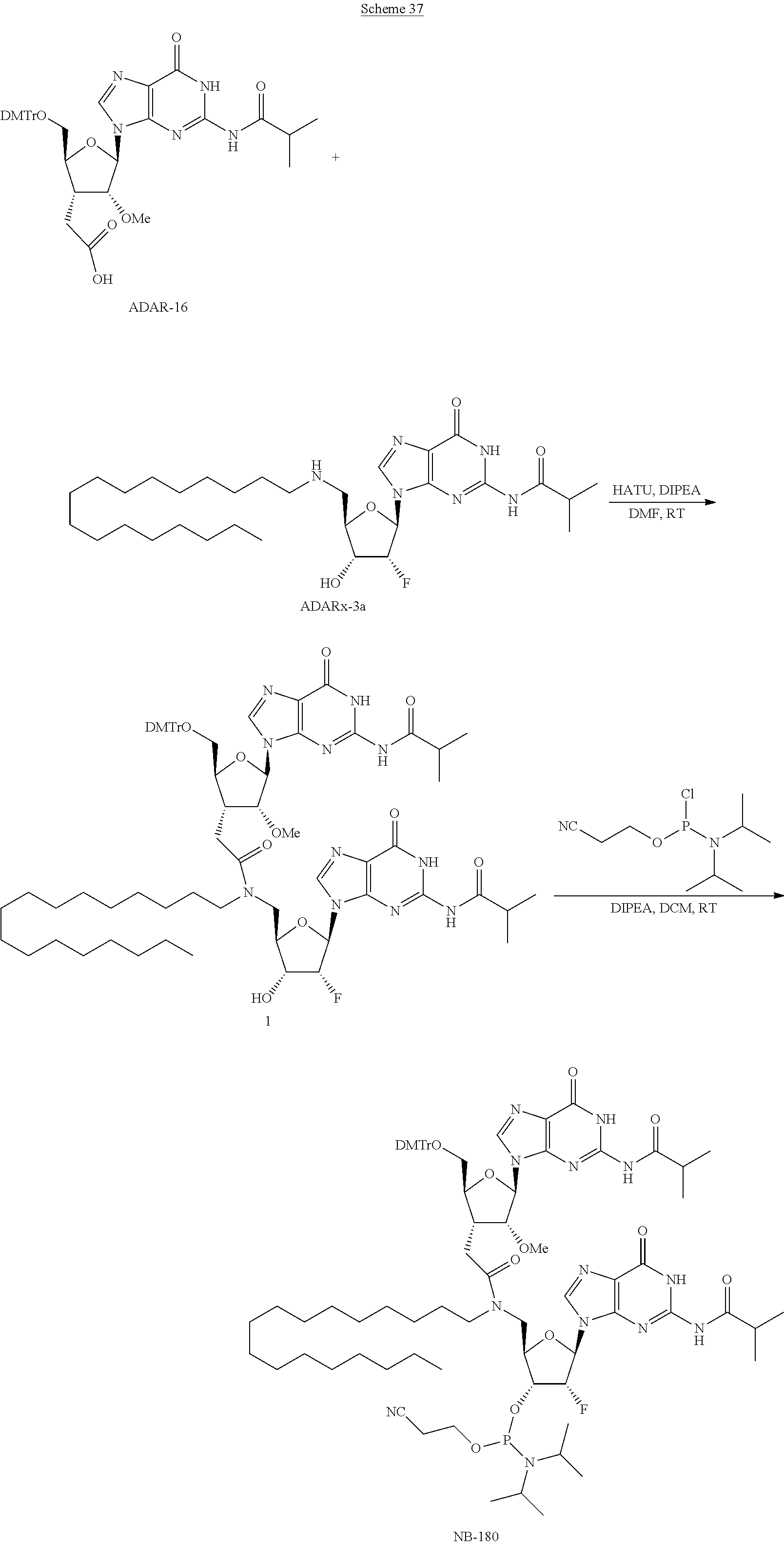

To a stirred solution of acid ADAR-016 (4 g, 5.626 mmol, 1 eq), amine ADARx-4a (3.66 g, 6.188 mmol, 1.1 eq) and HATU (3.2 g, 8.439 mmol, 1.5 eq) in DMF (30 mL) was added DIPEA (2.93 mL, 16.87 mmol, 3 eq) and the mixture was stirred for 3 h at room temperature, reaction mixture was added dropwise to a vigorously stirring water (250 ml), precipitated solids were filtered and washed with water, the solids were dried and re dissolved in DCM dried over Na₂SO₄ and concentrated, the residue was dissolved in EtOAc then purified by column chromatography using Ethyl acetate/MeOH, 0-5% as an eluent, pure fractions were combined and concentrated to obtain amide 1 (6.7 g, 84%) as a beige solid. Mass m/z 1285 (M+−1).

To a stirred solution of alcohol 1 (6.5 g, 5.05 mmol, 1 eq) and diisopropylethylamine (5.27 mL, 30.32 mmol, 6 eq), in DCM (80 mL), was added N, N-diisopropyl chlorophosphoramidite (3.3 mL 15.16 mmol, 3 eq) dropwise. The reaction mixture was stirred at room temperature for 30 min. Reaction mixture was quenched with Aq. Saturated NaHCO₃ solution (100 mL), extracted with DCM (100) ml, washed with brine (100 mL), dried over Na₂SO₄, and evaporated the crude product was loaded on to (pre-equilibrated with 1% Et₃N-EtOAc) Biotage silica gel column (100 g 20 μm), and purified by flash chromatography using MeOH/EtOAc 0-10% 10 CV, 10% 5 CV, containing 1% Et₃N as an additive, gave 2 g of pure product and 3 g of mixture with reagent. The mixture was concentrated and re dissolved in DCM 50 mL then 20 ml of Hexane was added slowly to get the precipitation, then the hexane was decanted, and repeated this process another time then the precipate was dissolved in DCM and combined with 2 g of the pure compound, concentrated and dried under high vacuum to obtain Phosphoramidite NB-180, 4.7 g, 62% as an off white solid. 94% purity by HPLC and 95% by P31, Mass (m/z 1486 (M+). ³¹P NMR (202, MHz, DMSO-d6) δ 150.72, 150.58, 150.51, 150.19, 150.00.

Oligonucleotide Comprising Dinucleotide NB-180 has been Synthesized Using the General Procedure Described in Example 1.

### Example 71: NB-181

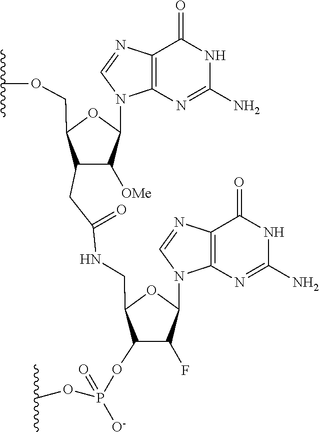

### Synthesis of NB-181

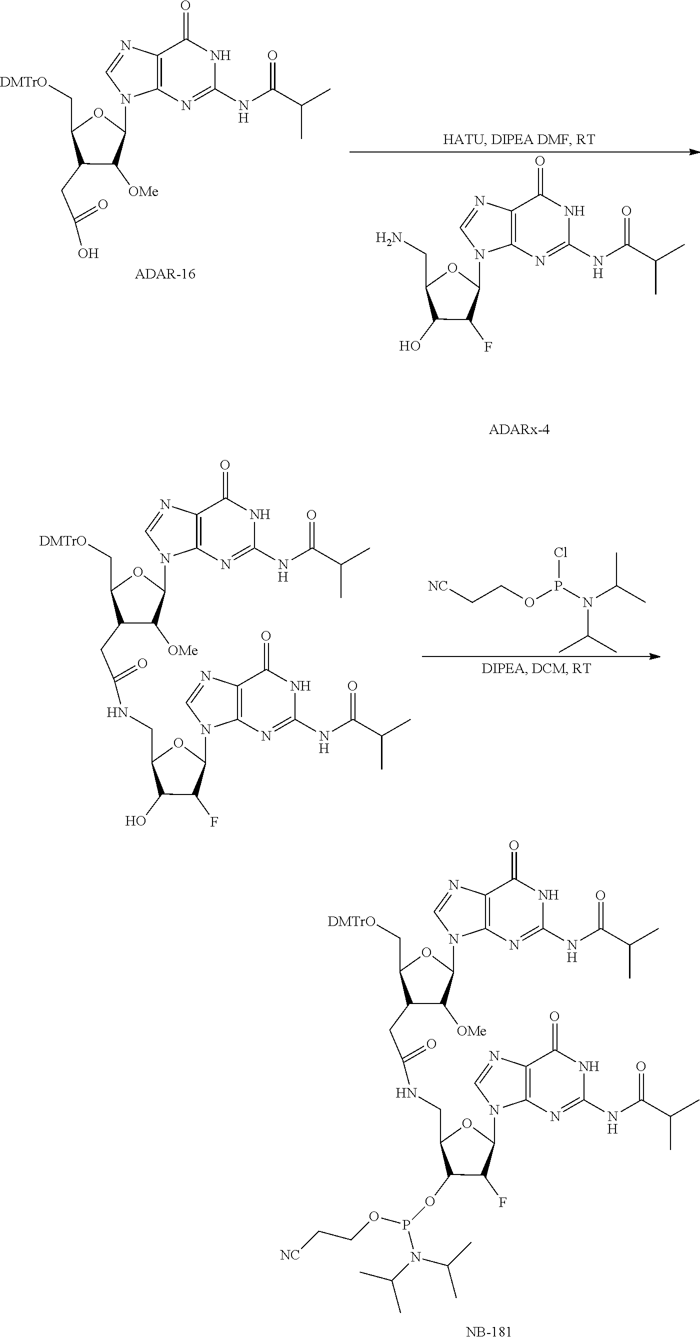

N-(9-((2R,3R,4R,5S)-5-((bis(4-methoxyphenyl)(phenyl)methoxy)methyl)-4-(2-((((2R,3R,4R,5R)-4-fluoro-3-hydroxy-5-(2-isobutyramido-6-oxo-1,6-dihydro-9H-purin-9-yl)tetrahydrofuran-2-yl)methyl)amino)-2-oxoethyl)-3-methoxytetrahydrofuran-2-yl)-6-oxo-6,9-dihydro-1H-purin-2-yl)isobutyramide. A solution of acid ADAR-016 (1 g, 1.405 mmol, 1 eq) amine ADARx-4 (0.548 g, 1.545 mmol, 1.1 eq) and HATU (0.801 g, 2.107 mmol, 1.5 eq) in DMF (40 mL) was added DIPEA (0.733 mL, 4.215 mmol, 3 eq), and the mixture was stirred for 1 hr at room temperature. Reaction mixture was added dropwise to a vigorously stirring solution of Aq. saturated NaHCO₃ (60 ml), precipitated solids were filtered and washed with water dried in a high vacuum overnight. Purified by flash chromatography (25 g 20 micron Biotage column) using MeOH/EtOAc, 0-20% 5 CV then 20% 10 CV as an eluent. Obtained amide (1.19 g, 80.68%) as a beige solid. LCMS m/z 1047 (M+−1) are corresponding with the product.

(2R,3R,4R,5R)-2-((2-((2S,3R,4R,5R)-2-((bis(4-methoxyphenyl)(phenyl) methoxy)methyl)-5-(2-isobutyramido-6-oxo-1,6-dihydro-9H-purin-9-yl)-4-methoxytetrahydrofuran-3-yl)acetamido)methyl)-4-fluoro-5-(2-isobutyramido-6-oxo-1,6-dihydro-9H-purin-9-yl)tetrahydrofuran-3-yl (2-cyanoethyl) diisopropylphosphoramidite. To a stirred solution of alcohol (1.188 g, 1.13 mmol, 1 eq) and diisopropylethylamine (1.182 mL, 6.801 mmol, 6 eq), in DCM (9 mL), was added N, N-diisopropyl chlorophosphoramidite (0.756 mL 3.4 mmol, 3 eq) dropwise. The reaction mixture was stirred at room temperature for 3 h. After 8 hr, additional 1 eq N, N-diisopropyl chlorophosphoramidite was added and stirred overnight. After overnight at RT, reaction mixture was quenched diluted with Aq. Saturated NaHCO₃ solution (100 mL), extracted with DCM (100) ml, washed with brine (100 mL), dried over Na₂SO₄, and evaporated the crude product was loaded on to (pre-equilibrated with 1% Et₃N-EtOAc) Biotage silica gel column (25 g 20 μm), and purified by flash chromatography using MeOH/EtOAc 0-10% 10 CV, 10% 5 CV, containing 1% Et₃N as an additive, gave 1.18 g (83.4%) of NB-181. ¹H NMR (500 MHz, d-DMSO): δ 12.1 (br, 3H), 8.1 (s, 1H), 8.0 (s, 1H), 7.4-7.2 (m, 9H), 6.8 (m, 4H), 6.2 (m, 1H), 6.0 (s, 1H), 5.6-5.5 (m, 1H), 4.5 (m, 1H), 4.2-4.0 (m, 4H), 3.8-3.6 (m, 11H), 3.6-3.2 (m, 26H), 2.8-2.6 (m, 6H), 2.6-2.5 (m, 3H), 2.0 (m, 2H), 1.2-1.8 (m, 32H). ³¹P NMR (202, MHz, DMSO-d6) δ 150.66, 150.59, 150.35, 150.32. Mass (ESI) m/z: [M+H]⁺ Cacld for 1248.53, found: 1248.3.

Oligonucleotide Comprising Dinucleotide NB-181 has been Synthesized Using the General Procedure Described in Example 1.

### Example 72: NB-182

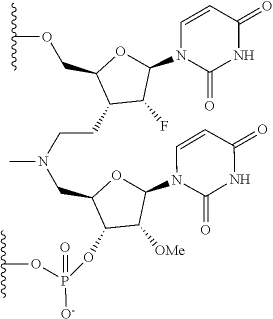 [SMILES: *OC[C@H]1O[C@@H](n2ccc(=O)[nH]c2=O)[C@H](F)[C@@H]1CCN(C)C[C@H]1O[C@@H](n2ccc(=O)[nH]c2=O)[C@H](OC)[C@@H]1OP(=O)([O-])O*]

### Synthesis of NB-182: (2R,3R,4R,5R)-2-(((2-((2S,3R,4R,5R)-2-((bis(4-methoxyphenyl)(phenyl)methoxy)methyl)-5-(2,4-dioxo-3,4-dihydropyrimidin-1(2H)-yl)-4-fluorotetrahydrofuran-3-yl)ethyl)(methyl)amino) methyl)-5-(2,4-dioxo-3,4-dihydropyrimidin-1(2H)-yl)-4-methoxytetrahydrofuran-3-yl (2-cyanoethyl)diisopropylphosphoramidite

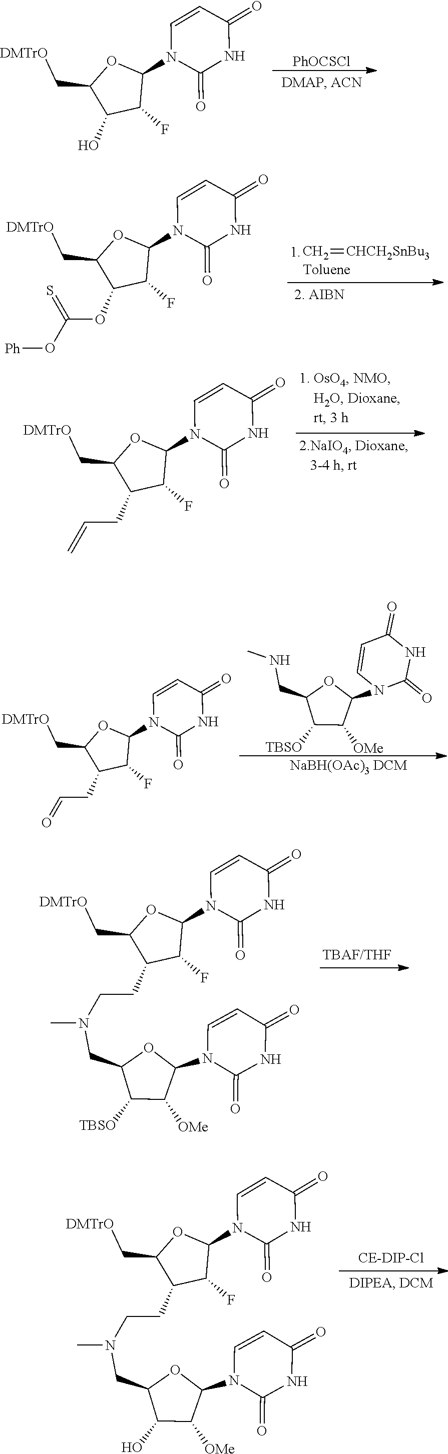

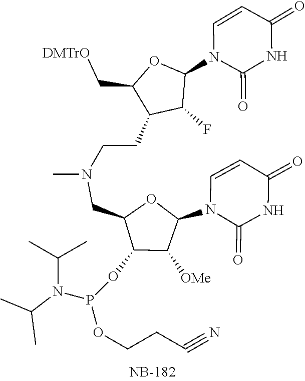

### Step 1: 0-((2R,3R,4R,5R)-2-((bis(4-methoxyphenyl)(phenyl)methoxy)methyl)-5-(2,4-dioxo-3,4-dihydro pyrimidin-1(2H)-yl)-4-fluorotetrahydrofuran-3-yl)O-phenyl carbonothioate

A solution of 2′-deoxy-5′-O-DMT-2′-fluorouridine (50 g, 91 mmol) in ACN (500 mL) was treated with DMAP (22 g, 182 mmol) followed by the addition of phenyl chlorothionoformate (23.6 g, 136 mmol) in portions at room temperature. The resulting mixture was stirred for 2 hours at room temperature under argon atmosphere. The reaction was quenched with sat. NaHCO₃ (aq., 50 mL) at 0° C. The resulting mixture was extracted with EtOAc (3×500 mL). The combined organic layers were washed with brine (3×300 mL), dried over anhydrous Na₂SO₄. After filtration, the filtrate was concentrated under reduced pressure. The residue was purified by silica gel column chromatography, eluted with PE/EA (1:1) to afford the titled compound (48 g, 77%) as a yellow solid. MS (ESI, negative mode) m/z=683.1 [M−H]⁻.

### Step 2: 1-((2R,3R,4R,5S)-4-allyl-5-((bis(4-methoxyphenyl)(phenyl)methoxy)methyl)-3-fluorotetra hydrofuran-2-yl)pyrimidine-2,4(1H,3H)-dione

To a stirred mixture of O-((2R,3R,4R,5R)-2-((bis(4-methoxyphenyl)(phenyl)methoxy)methyl)-5-(2,4-dioxo-3,4-dihydropyrimidin-1(2H)-yl)-4-fluorotetrahydrofuran-3-yl)O-phenyl carbonothioate (35 g, 51 mmol) and tributyl(prop-2-en-1-yl)stannane (67.7 g, 204 mmol) in toluene (350 mL) was added AIBN (6.71 g, 40.9 mmol) in portions at room temperature under argon atmosphere. The resulting mixture was stirred for 4 h at 80° C. under argon atmosphere. The resulting mixture was concentrated under vacuum. The residue was purified by silica gel column chromatography, eluted with 0.5% TEA in PE/EA (1:1, v/v) to afford the titled compound (11.0 g, 37%) as a white solid. MS (ESI, negative mode): m/z=571.5 [M−H]⁻.

### Step 3: 2-((2S,3R,4R,5R)— 2-((bis(4-methoxyphenyl)(phenyl) methoxy)methyl)-5-(2,4-dioxo-3,4-dihydropyrimidin-1(2H)-yl)-4-fluorotetrahydrofuran-3-yl)acetaldehyde

To a stirred solution of 1-((2R,3R,4R,5S)-4-allyl-5-((bis(4-methoxyphenyl)(phenyl)methoxy) methyl)-3-fluorotetrahydrofuran-2-yl)pyrimidine-2,4(1H,3H)-dione (4.2 g, 7.3 mmol) in dioxane (84 mL) were added OsO₄ (373 mg, 1.47 mmol) and NMO (1.03 g, 8.80 mmol) in portions at RT under argon atmosphere. The resulting mixture was stirred for 2 hours at room temperature under argon atmosphere with light-protection using a sheet of Al foil. The reaction was quenched with aqueous NaHCO₃ (sat, 20 mL) at room temperature. The aqueous layer was extracted with DCM (1 L). The combined organic layers were concentrated under reduced pressure. The residue was dissolved in dioxane (84 mL). To the above mixture was added NaIO₄ (1.88 g, 8.80 mmol) in H₂O (4.20 mL) dropwise. The resulting mixture was stirred for additional 1.5 hour at room temperature. The resulting mixture was diluted with DCM (500 mL). The reaction was quenched with aqueous NaHCO₃ (sat, 100 mL) at 0° C. The resulting mixture was extracted with DCM (3×200 mL), dried over anhydrous Na₂SO₄. After filtration, the filtrate was concentrated under reduced pressure to afford the titled compound (4.1 g, 95% yield) as an off-white solid which was used in the next step without purification. MS (ESI, negative mode) m/z=573.2 [M−H]⁻.

### Step 4: 1-((2R,3R,4R,5S)-5-((bis(4-methoxyphenyl)(phenyl)methoxy)methyl)-4-(2-((((2R,3R,4R,5R)-3-((tert-butyldimethylsilyl)oxy)-5-(2,4-dioxo-3,4-dihydropyrimidin-1(2H)-yl)-4-methoxytetra hydrofuran-2-yl)methyl)(methyl)amino)ethyl)-3-fluorotetrahydrofuran-2-yl)pyrimidine-2,4(1H,3H)-dione

A solution of 2-((2S,3R,4R,5R)-2-((bis(4-methoxyphenyl)(phenyl) methoxy)methyl)-5-(2,4-dioxo-3,4-dihydropyrimidin-1(2H)-yl)-4-fluorotetrahydrofuran-3-yl)acetaldehyde (3.00 g, 3.63 mmol, crude) and 1-((2R,3R,4R,5R)-4-((tert-butyldimethylsilyl)oxy)-3-methoxy-5-((methylamino)-methyl)tetrahydrofuran-2-yl)pyrimidine-2,4(1H,3H)-dione (1.44 g, 2.57 mmol) in DCM (30 mL), and was added NaBH(OAc)₃ (990 mg, 3.11 mmol) batchwise. The resulting solution was stirred for 1 hour at room temperature under nitrogen atmosphere. The reaction was quenched by addition of water (20 mL). The resulting mixture was extracted with EA (3×500 mL). The combined organic layers were washed with brine (50 mL), dried over anhydrous Na₂SO₄. After filtration, the filtrate was concentrated under reduced pressure. The residue was purified by silica gel column chromatography, eluted with MeOH/DCM (13%) to afford the titled compound (2.0 g, 59% yield) as a white solid. MS (ESI): m/z=944.7 [M+H]⁺.

Step 5: 1-((2R,3R,4R,5S)-5-((bis(4-methoxyphenyl)(phenyl)methoxy)methyl)-4-(2-((((2R,3R,4R,5R)-5-(2,4-dioxo-3,4-dihydropyrimidin-1(2H)-yl)-3-hydroxy-4-methoxytetrahydrofuran-2-yl)methyl) (methyl)amino)ethyl)-3-fluorotetrahydrofuran-2-yl)pyrimidine-2,4(1H,3H)-dione

A solution of 1-((2R,3R,4R,5S)-5-((bis(4-methoxyphenyl)(phenyl)methoxy)methyl)-4-(2-((((2R,3R,4R,5R)-3-((tert-butyldimethylsilyl)oxy)-5-(2,4-dioxo-3,4-dihydropyrimidin-1(2H)-yl)-4-methoxytetrahydrofuran-2-yl)methyl)(methyl)amino)ethyl)-3-fluorotetrahydrofuran-2-yl) pyrimidine-2,4(1H,3H)-dione (3.0 g, 3.2 mmol) and TBAF (1M in THF, 4.8 mL, 4.8 mmol) in THF (30 mL) was stirred for 1 hour at room temperature under nitrogen atmosphere. The resulting mixture was extracted with EA (2×500 mL). The combined organic layers were washed with water (50 mL) and dried over anhydrous Na₂SO₄. After filtration, the filtrate was concentrated under reduced pressure. The residue was purified by reversed phase chromatography (column, C18; mobile phase, ACN in water, 10 to 90% gradient in 30 min; detector, UV 254 nm) to afford the titled compound (2.44 g, 86.5% yield) as an off-white solid. MS (ESI): m/z=830.3 [M+H]⁺.

### Step 6: (2R,3R,4R,5R)-2-(((2-((2S,3R,4R,5R)-2-((bis(4-methoxyphenyl)(phenyl)methoxy) methyl)-5-(2,4-dioxo-3,4-dihydropyrimidin-1(2H)-yl)-4-fluorotetrahydrofuran-3-yl)ethyl) (methyl)amino) methyl)-5-(2,4-dioxo-3,4-dihydropyrimidin-1(2H)-yl)-4-methoxytetrahydrofuran-3-yl(2-cyanoethyl) diisopropylphosphoramidite

Cyanoethyl N,N-diisopropylchlorophosphoramidite (1.5 mL, 6.5 mmol) was added to a solution of 1-((2R,3R,4R,5S)-5-((bis(4-methoxyphenyl)(phenyl)methoxy)methyl)-4-(2-((((2R,3R,4R,5R)-5-(2,4-dioxo-3,4-dihydropyrimidin-1(2H)-yl)-3-hydroxy-4-methoxytetrahydrofuran-2-yl)methyl) (methyl)amino)ethyl)-3-fluorotetrahydrofuran-2-yl)pyrimidine-2,4(1H,3H)-dione (1.8 g, 2.2 mmol) and DIPEA (2.4 mL, 13 mmol) in DCM (15 mL) dropwisely at room temperature. The reaction was stirred for 3 hours. The reaction was quenched by pouring the mixture to an aqueous solution of NaHCO₃ (10 mL) and extracted with DCM (2×500 mL). The combined organic layer was separated, dried with Na₂SO₄, filtered and concentrated. The crude was purified by flash column on silica gel column (column with pretreated with 1% Et₃N with hexane) with 0 to 100% EA/H to obtain the product with impurity related to the phosphorous reagent. Titiration with hexane and DCM to afford the titled compound NB-182 (1.5 g, 71% yield) as a white solid. ³¹P NMR (202 MHz, CDCl₃) δ 149.73, 149.49. MS (ESI): 1030.1[M+H]⁺, 1052.0[M+Na]⁺.

Oligonucleotide Comprising Dinucleotide NB-182 has been Synthesized Using the General Procedure Described in Example 1.

### Example 73: NB-183

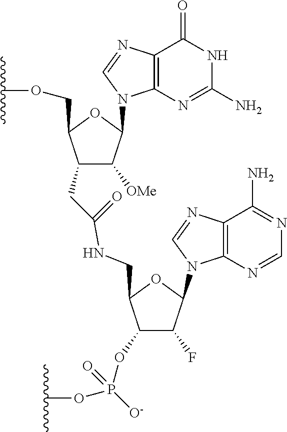 [SMILES: *OC[C@H]1O[C@@H](n2cnc3c(=O)[nH]c(N)nc32)[C@H](OC)[C@@H]1CC(=O)NC[C@H]1O[C@@H](n2cnc3c(N)ncnc32)[C@H](F)[C@@H]1OP(=O)([O-])O*]

### Synthesis of NB-183

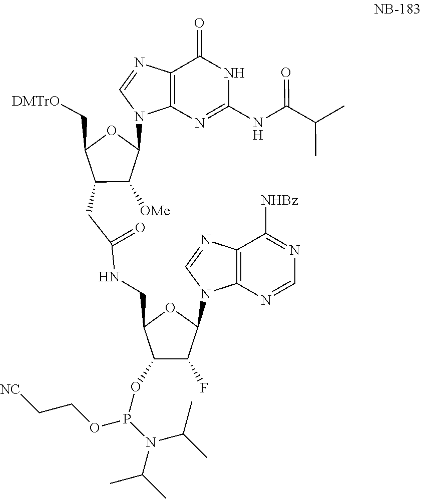

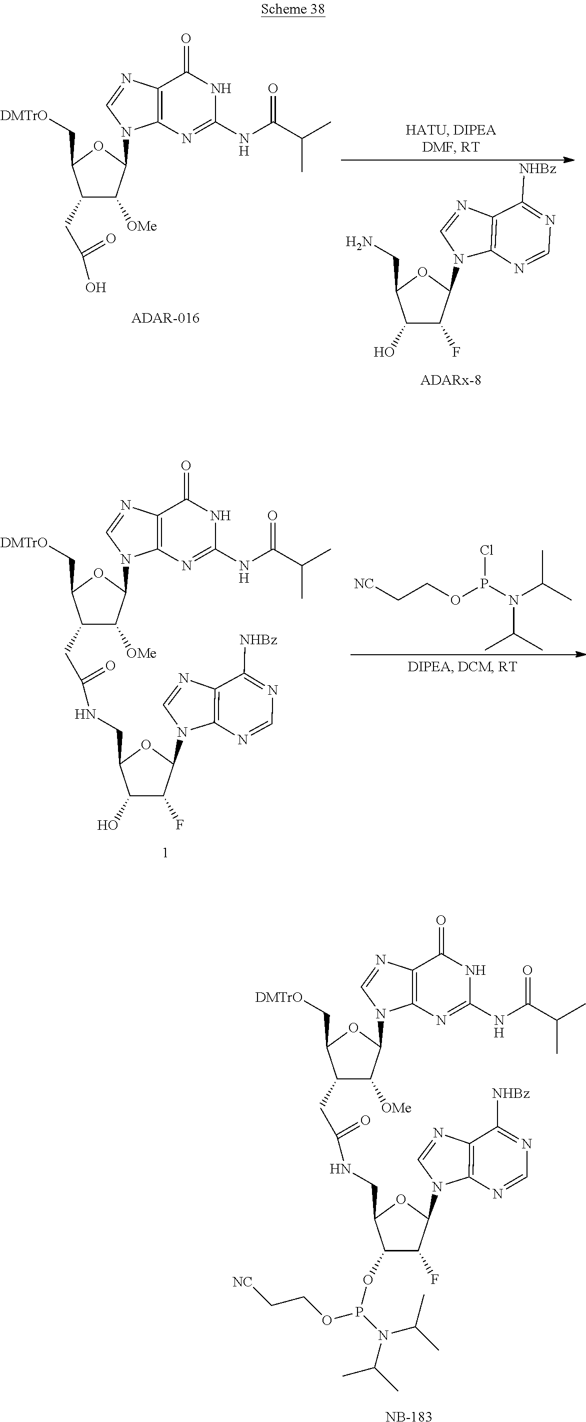

To a stirred solution of acid ADAR-016 (5 g, 7.032 mmol, 1 eq), amine ADARx-8 (2.878 g, 7.736 mmol, 1.1 eq) and HATU (4 g, 10.5 mmol, 1.5 eq) in DMF (30 mL) was added DIPEA (3.6 mL, 21 mmol, 3 eq) and the mixture was stirred for 1 h at room temperature, reaction mixture was added dropwise to a vigorously stirring brine solution (250 ml), precipitated solids were filtered and washed with water, the solids were dried and re dissolved in DCM dried over Na₂SO₄ and concentrated, the residue was dissolved in DCM then purified by column chromatography using Ethyl acetate/MeOH, 0-20% as an eluent, pure fractions were combined and concentrated to obtain amide 1 (6.32 g, 76%) as a beige solid (>90% purity). LCMS m/z 1088 (M+Na).

To a stirred solution of alcohol 1 (6.28 g, 5.891 mmol, 1 eq) and diisopropylethylamine (6.15 mL, 35.34 mmol, 6 eq), in DCM (80 mL), was added N, N-diisopropyl chlorophosphoramidite (3.9 mL 17.6 mmol, 3 eq) dropwise. The reaction mixture was stirred at room temperature for 5 h min. Reaction mixture was quenched with Aq. Saturated NaHCO₃ solution (100 mL), extracted with DCM (200) ml, washed with brine (100 mL), dried over Na₂SO₄, and evaporated the crude product was loaded on to (pre-equilibrated with 1% Et₃N-EtOAc) Biotage silica gel column (100 g 20 μm), and purified by flash chromatography using MeOH/EtOAc 2-10% 3 CV, 10% 10 CV, containing 1% Et₃N as an additive, gave 4.1 g of pure product and 4 g of mixture with reagent. The mixture was concentrated and re dissolved in DCM 50 mL then 50 ml of Hexane was added slowly to get the precipitation, then the hexane was decanted, and repeated this process another time then the precipate was dissolved in DCM and combined with 4.1 g of the pure compound, concentrated and dried under high vacuum to obtain Phosphoramidite NB-183, 6.38 g, 85% as an off white solid. 94% purity by LCMS, Mass (m/z 1288 (M++Na). ³¹P NMR (202, MHz, DMSO-d6) δ 150.52, 150.49, 149.97, 149.90.

Oligonucleotide Comprising Dinucleotide NB-183 has been Synthesized Using the General Procedure Described in Example 1.

### Example 74: NB-184

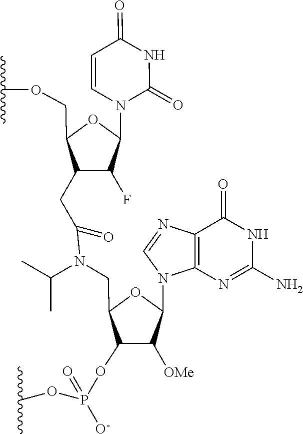

### Synthesis of NB-184

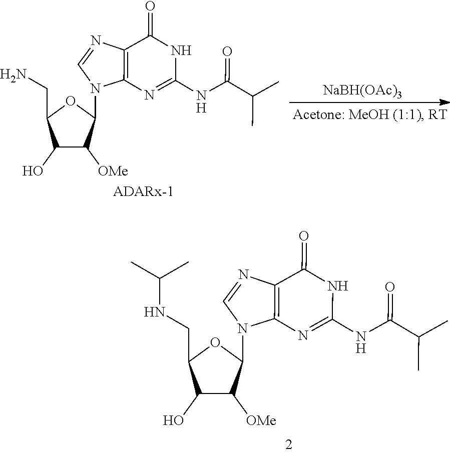

N-(9-((2R,3R,4R,5R)-4-hydroxy-5-((isopropylamino)methyl)-3-methoxytetrahydrofuran-2-yl)-6-oxo-6,9-dihydro-1H-purin-2-yl)isobutyramide. To a solution of amine (5.0 g, 13.6 mmol, 1 eq) in Acetone:MeOH (68 mL:68 mL) was stirred at RT for 30 min. Then NaBH(OAc)₃ added (3.472 g, 16.376 mmol, 1.2 eq) stirred at RT for 1 hr. Then the reaction mixture was filtered, washed with MeOH, concentrated. Then Mixture was diluted with water, extracted with DCM:MeOH (1:1) 200 mL five times, dried over Na₂SO₄ filtered concentrated, obtained 3.34 g 60% of desired product as white solid. LCMS m/z M-+1 corresponds to the desired product.

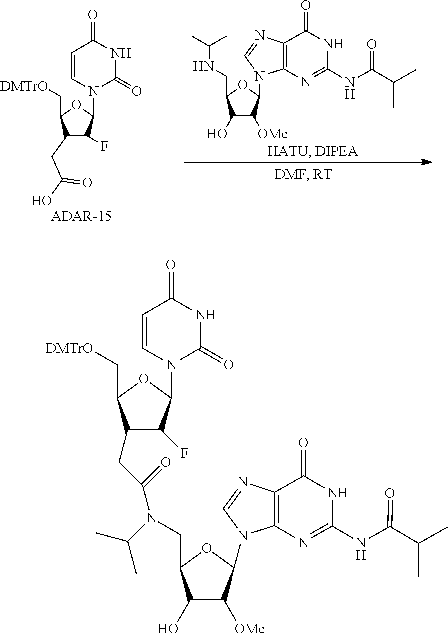

N-(9-((2R,3R,4R,5R)-5-((2-((2S,3R,4R,5R)-2-((bis(4-methoxyphenyl)(phenyl)methoxy)methyl)-5-(2,4-dioxo-3,4-dihydropyrimidin-1(2H)-yl)-4-fluorotetrahydrofuran-3-yl)-N-isopropylacetamido)methyl)-4-hydroxy-3-methoxytetrahydrofuran-2-yl)-6-oxo-6,9-dihydro-1H-purin-2-yl)isobutyramide A solution of acid (2 g, 3.39 mmol, 1 eq), SM-125-5 (1.52 g, 3.73 mmol, 1.1 eq) and HATU (1.93 g, 5.09 mmol, 1.5 eq) in DMF (16.5 mL) was added DIPEA (1.77 mL, 10.2 mmol, 3 eq), and the mixture was stirred for 1 hr 30 min at room temperature. Reaction mixture was added to a stirring solution of sat. NaHCO₃, precipitated solids were filtered and washed with water and dried under high vac overnight (3.08 g crude). Purified by flash chromatography (25 g 20 micron Biotage column) using MeOH/EtOAc, 0-20% 5 CV then 20% 10 CV as an eluent. Obtained amide (1.81 g, 54.4%) as a beige solid. LCMS m/z 980.05 (M+−1) are corresponding with the product.

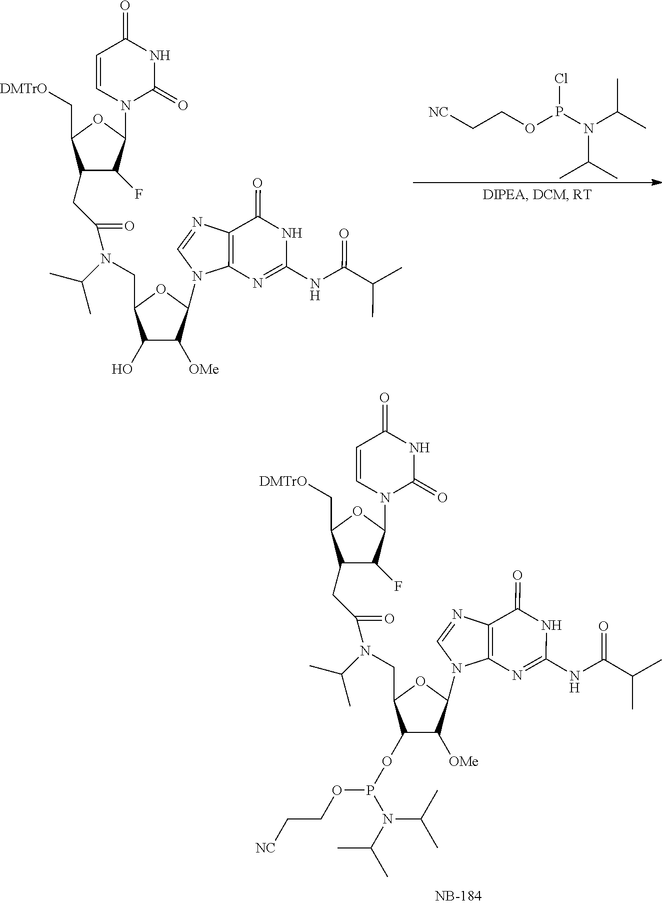

(2R,3R,4R,5R)-2-((2-((2S,3R,4R,5R)-2-((bis(4-methoxyphenyl)(phenyl)methoxy)methyl)-5-(2,4-dioxo-3,4-dihydropyrimidin-1(2H)-yl)-4-fluorotetrahydrofuran-3-yl)-N-isopropylacetamido)methyl)-5-(2-isobutyramido-6-oxo-1,6-dihydro-9H-purin-9-yl)-4-methoxytetrahydrofuran-3-yl (2-cyanoethyl) diisopropylphosphoramidite. To a stirred solution of alcohol (1.328 g, 1.354 mmol, 1 eq) and diisopropylethylamine (1.412 mL, 8.122 mmol, 6 eq), in DCM (10.7 mL), was added N, N-diisopropyl chlorophosphoramidite (0.903 mL, 4.061 mmol, 3 eq) dropwise. The reaction mixture was stirred at room temperature for 2 hr. Quench with Sat NaHCO₃, extracted with DCM, Dried over Na₂SO₄ and concentrated. Biotage silica gel column (25 g 20 μm), and purified by flash chromatography using MeOH/EtOAc 0-10% 10 CV, 10% 5 CV, containing 1% Et₃N as an additive, gave 1.18 g product. Triturated with DCM/Hexane 3× afforded 960 mg (60%) of NB-184. ¹H NMR (500 MHz, d-DMSO): δ 11.5 (br, 3H), 8.4-8.2 (m, 1H), 7.8 (m, 1H), 7.4-7.0 (m, 10H), 6.6-6.4 (m, 4H), 5.8 (m, 2H), 5.4-5.0 (m, 2H), 4.8-4.5 (m, 2H), 4.5 (m, 1H), 4.3-3.8 (m, 4H), 3.8-3.5 (m, 11H), 3.4-3.0 (m, 30H), 2.8-2.5 (m, 5H), 2.6-2.5 (m, 3H), 2.0 (m, 2H), 1.0 (m, 30H). ³¹P NMR (202, MHz, DMSO-d6) δ 150.46, 150.16, 150.02. Mass (ESI) m/z: [M+H]⁺ Cacld for 1282.53, found: 1282.3.

Oligonucleotide Comprising Dinucleotide NB-184 has been Synthesized Using the General Procedure Described in Example 1.

### Example 75: NB-185

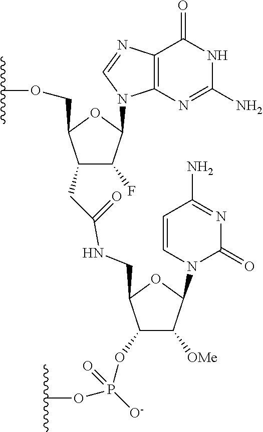 [SMILES: *OC[C@H]1O[C@@H](n2cnc3c(=O)[nH]c(N)nc32)[C@H](F)[C@@H]1CC(=O)NC[C@H]1O[C@@H](n2ccc(N)nc2=O)[C@H](OC)[C@@H]1OP(=O)([O-])O*]

### Synthesis of NB-185

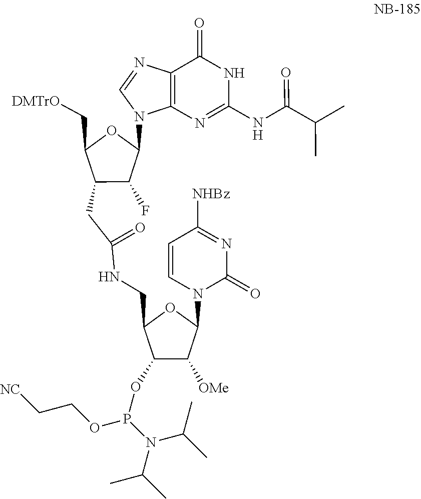

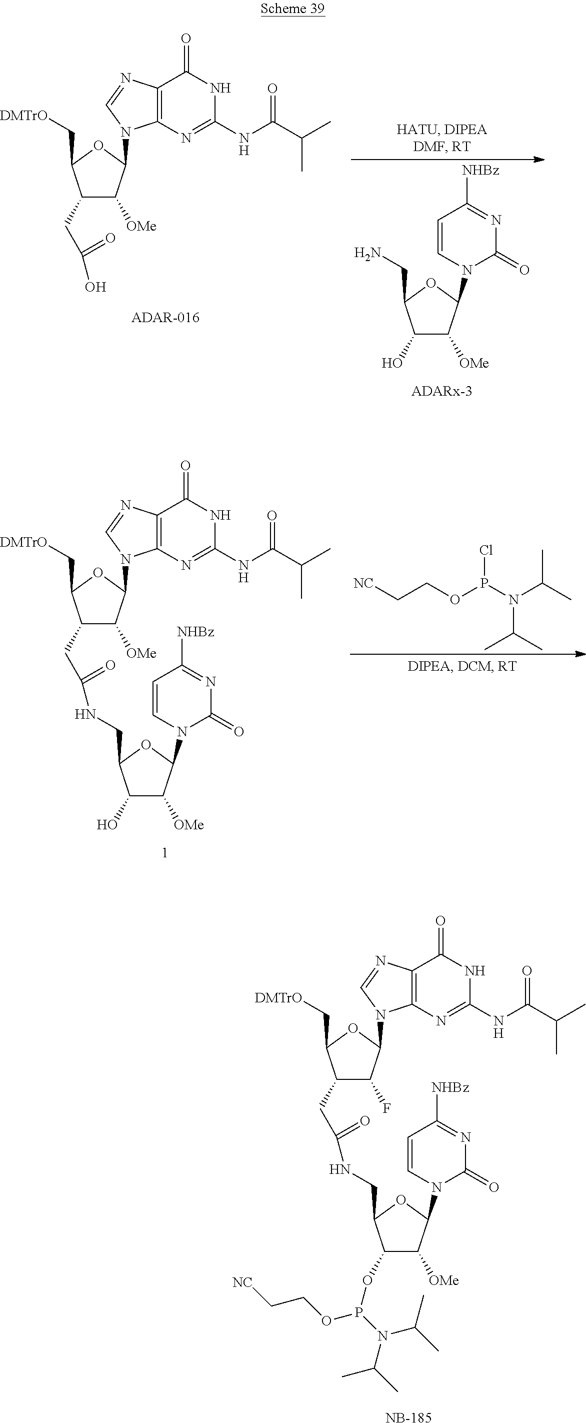

To a stirred solution of acid ADAR-016 (5 g, 7.153 mmol, 1 eq), amine ADARx-3 (2.83 g, 7.86 mmol, 1.1 eq) and HATU (4.07 g, 10.73 mmol, 1.5 eq) in DMF (30 mL) was added DIPEA (3.73 mL, 21.45 mmol, 3 eq) and the mixture was stirred for 3 h at room temperature, reaction mixture was added dropwise to a vigorously stirring brine (250 ml), precipitated solids were filtered and washed with water, the solids were dried and re dissolved in DCM dried over Na₂SO₄ and concentrated, the residue was dissolved in DCM then purified by column chromatography using DCM/MeOH, 0-20% as an eluent, pure fractions were combined and concentrated to obtain amide 1 (6.25 g, 76%) as a beige solid. LCMS m/z 1043 (M+1).

To a stirred solution of alcohol 1 (6.23 g, 5.979 mmol, 1 eq) and diisopropylethylamine (6.23 mL, 35.87 mmol, 6 eq), in DCM (80 mL), was added N, N-diisopropyl chlorophosphoramidite (4 mL 17.94 mmol, 3 eq) dropwise. The reaction mixture was stirred at room temperature for 5 h. Reaction mixture was quenched with Aq. Saturated NaHCO₃ solution (100 mL), extracted with DCM (200) ml, washed with brine (100 mL), dried over Na₂SO₄, and evaporated the crude product was loaded on to (pre-equilibrated with 1% Et₃N-EtOAc) Biotage silica gel column (100 g 20 μm), and purified by flash chromatography using MeOH/EtOAc 2-10% 3 CV, 10% 10 CV, containing 1% Et₃N as an additive, gave mixture with reagent. The mixture was concentrated and re dissolved in DCM 50 mL then 50 ml of Hexane was added slowly to get the precipitation, then the hexane was decanted, resulting gum was concentrated and dried under high vacuum to obtain Phosphoramidite NB-185, 5.20 g, 70% as an off white solid. 90% purity by HPLC, Mass (m/z 1264 (M++Na). ³¹P NMR (202, MHz, DMSO-d6) δ 149.95, 149.75.

Oligonucleotide Comprising Dinucleotide NB-185 has been Synthesized Using the General Procedure Described in Example 1.

### Example 76: NB-186

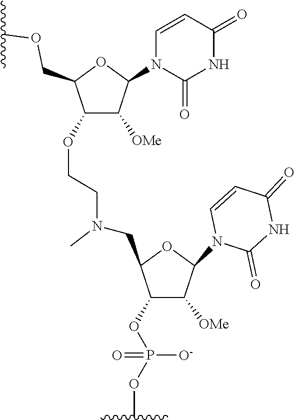 [SMILES: *OC[C@H]1O[C@@H](n2ccc(=O)[nH]c2=O)[C@H](OC)[C@@H]1OCCN(C)C[C@H]1O[C@@H](n2ccc(=O)[nH]c2=O)[C@H](OC)[C@@H]1OP(=O)([O-])O*]

### Synthesis of NB-186: (2R,3R,4R,5R)-2-(((2-(((2R,3R,4R,5R)-2-((bis(4-methoxyphenyl)(phenyl)methoxy)methyl)-5-(2,4-dioxo-3,4-dihydropyrimidin-1(2H)-yl)-4-methoxytetrahydrofuran-3-yl)oxy)ethyl)(methyl)amino)methyl)-5-(2,4-dioxo-3,4-dihydropyrimidin-1(2H)-yl)-4-methoxytetrahydrofuran-3-yl (2-cyanoethyl) diisopropylphosphoramidite

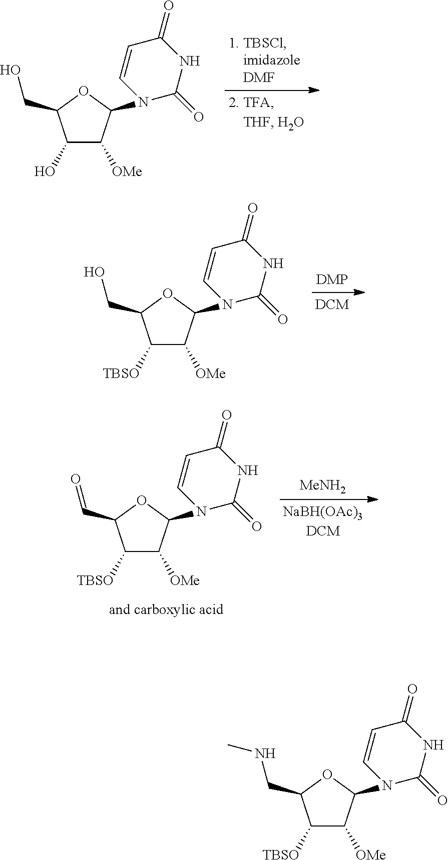

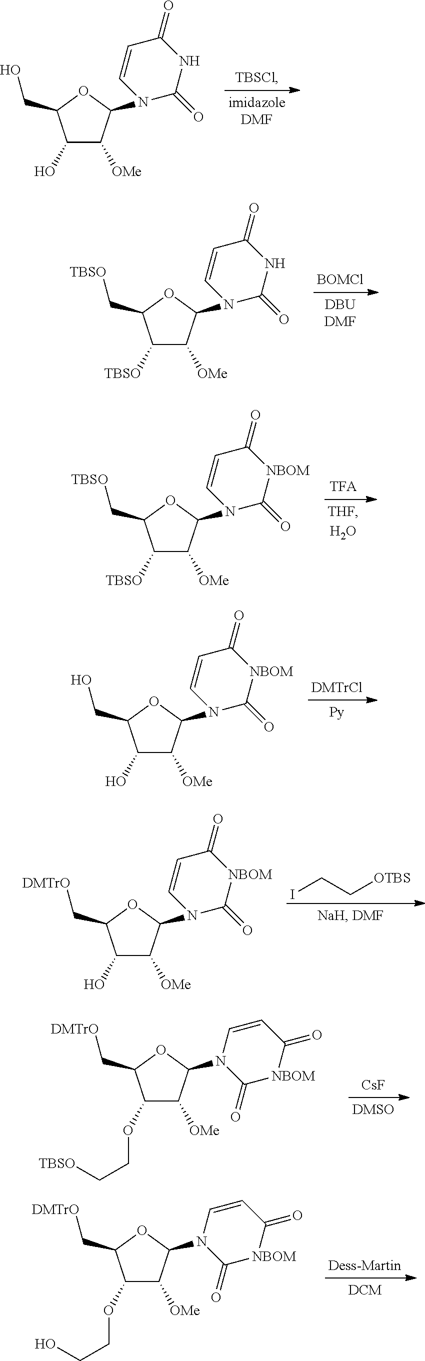

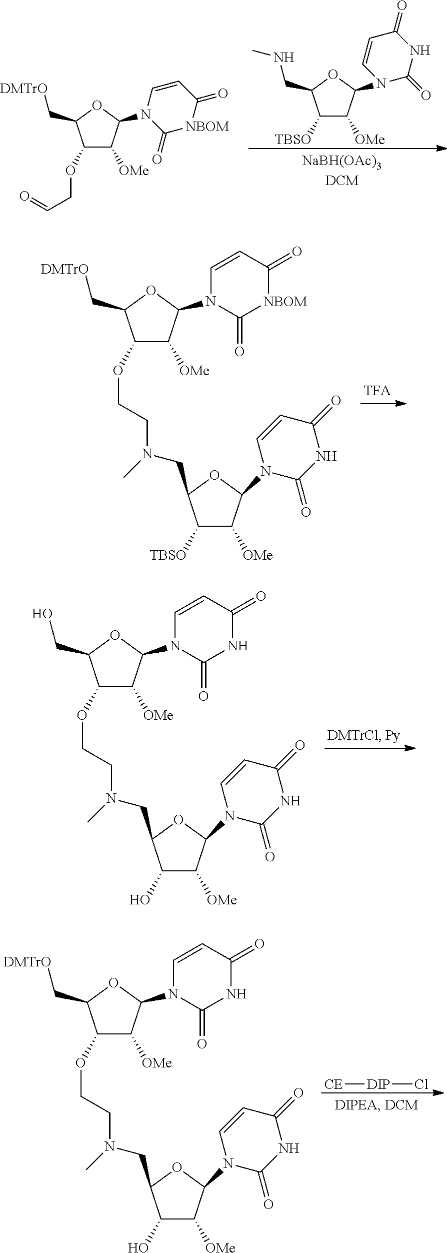

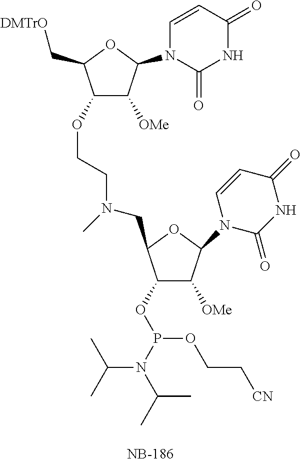

### Step 1: 1-((2R,3R,4R,5R)-4-((tert-butyldimethylsilyl)oxy)-5-(hydroxymethyl)-3-methoxy-tetrahydrofuran-2-yl)pyrimidine-2,4(1H,3H)-dione

A solution of 2′-O-methyluridine (80.0 g, 310 mmol) was dissolved in DMF (400 mL), followed by the addition of imidazole (84.0 g, 1230 mmol) and TBSCI (280 g, 1858 mmol) in portions at 0° C. The resulting mixture was stirred for 12 hours at room temperature under Ar atmosphere. The reaction was quenched by the addition of water (100 mL) at 0° C. The resulting mixture was extracted with EtOAc (3×200 mL). The combined organic layers were washed with brine (2×100 mL), dried over anhydrous Na₂SO₄. After filtration, the filtrate was concentrated under reduced pressure to afford the crude, bis-TBS product (280 g, quantitative yield) which was used in the next step without purification. To a flask containing bis-TBS product (280 g, 310 mmol) was added THF (1400 mL) followed by the addition of H₂O (700 mL) and TFA (700 mL) at 0° C. The mixture was stirred at 0° C. for 1 hour. The pH pf the mixture was adjusted to 7 with ammonium hydroxide solution. The resulting mixture was extracted with EtOAc (3×1 L). The organic layer was separated, the combined organic layers were washed with brine (3×700 mL), dried over anhydrous Na₂SO₄. After filtration, the filtrate was concentrated under reduced pressure. The residue was purified by silica gel column chromatography, eluted with PE/EA (0 to 100%) to afford the titled compound (42.0 g, 34.6% yield) as a white solid. MS(ESI) m/z=373.1 [M+H]⁺; 395.2 [M+Na]⁺.

### Step 2: (2S,3S,4R,5R)-3-((tert-butyldimethylsilyl)oxy)-5-(2,4-dioxo-3,4-dihydropyrimidin-1(2H)-yl)-4-methoxytetrahydrofuran-2-carbaldehyde

To a solution of 1-((2R,3R,4R,5R)-4-((tert-butyldimethylsilyl)oxy)-5-(hydroxymethyl)-3-methoxy-tetrahydrofuran-2-yl)pyrimidine-2,4(1H,3H)-dione (42.0 g, 113 mmol) in DCM (840 mL) were added Dess-Martin periodinane (55.00 g, 129.7 mmol) at 0° C. and the reaction was stirred at RT for overnight. The reaction was diluted with EA (4 L). The organic layer was separated and the aqueous phase was set aside. The organic phase was washed with saturated NaHCO₃ solution (500 mL×3). This organic layer was washed with saturated Na₂S₂O₃ solution (500 mL×3), brine (500 mL×3) and dried over anhydrous Na₂SO₄. After filtration, the filtrate was concentrated in vacuo to afford the title compound (42 g, 80% purity, 80% yield) as a white solid. MS(ESI): m/z=371.3 [M+H]⁺. For the aqueous phase that was set aside, the pH of this layer after the was adjusted to 5-6 with 1N HCl and was extracted with EA (3×1 L). The EA layer was dried by Na₂SO₄, filtered and concentrated to afford the crude product as an over-oxidized product (2S,3S,4R,5R)-3-((tert-butyldimethylsilyl)oxy)-5-(2,4-dioxo-3,4-dihydro-pyrimidin-1(2H)-yl)-4-methoxytetrahydrofuran-2-carboxylic acid (7.0 g, 18 mmol, 16% yield) as a white solid. MS(ESI): m/z=387.2 [M+H]⁺.

### Step 3: 1-((2R,3R,4R,5R)-4-((tert-butyldimethylsilyl)oxy)-3-methoxy-5-((methylamino)methyl)-tetrahydrofuran-2-yl)pyrimidine-2,4(1H,3H)-dione

To a stirred solution of (2S,3S,4R,5R)-3-[(tert-butyldimethylsilyl)oxy]-5-(2,4-dioxo-3H-pyrimidin-1-yl)-4-methoxyoxolane-2-carbaldehyde (15.0 g, 40.5 mmol) in MeOH (150 mL) were added methylamine (140 mL, 1.21 mol, 30% in EtOH) and NaBH(OAc)₃ (85.8 g, 405 mmol) in portions at room temperature. The resulting mixture was stirred for 3 hours at room temperature under nitrogen atmosphere. The resulting mixture was diluted with ethyl acetate (3 L). The combined organic layers were washed with water (3×200 mL), sat. NaHCO₃ (aq., 2×200 mL), brine (200 mL), dried over anhydrous Na₂SO₄, filtered and concentrated. The residue was purified by silica gel column chromatography, eluted with CH₂Cl₂/MeOH (10:1) to afford the titled compound (4.0 g, 26% yield) as a yellow solid. MS(ESI): m/z=386.1 [M+H]⁺.

### Step 4: 1-((2R,3R,4R,5R)-4-((tert-butyldimethylsilyl)oxy)-5-(((tert-butyldimethylsilyl)oxy)methyl)-3-methoxytetrahydrofuran-2-yl)pyrimidine-2,4(1H,3H)-dione

A solution of 2′-O-methyluridine (20.0 g, 77.4 mmol) in DMF was treated with imidazole (21.09 g, 309.8 mmol) at room temperature under nitrogen atmosphere followed by the addition of TBSCl (70.0 g, 464 mmol) in portions at 0° C. The resulting mixture was stirred for 2 hours at room temperature under argon atmosphere. The reaction was quenched with water (100 mL) at room temperature. The aqueous layer was extracted with EtOAc (3×500 mL). The combined organic layers were washed with water (100 mL) and brine (100 mL). The organic layer was dried Na₂SO₄. The resulting mixture was concentrated under vacuum to afford the titled compound (38 g, 100% yield). The crude product was used in the next step directly without further purification. MS(ESI): m/z=487.2 [M+H]⁺.

### Step 5: 3-((benzyloxy)methyl)-1-((2R,3R,4R,5R)-4-((tert-butyldimethylsilyl)oxy)-5-(((tert-butyldi-methylsilyl)oxy)methyl)-3-methoxytetrahydrofuran-2-yl)pyrimidine-2,4(1H,3H)-dione

A solution of 1-((2R,3R,4R,5R)-4-((tert-butyldimethylsilyl)oxy)-5-(((tert-butyldimethylsilyl)oxy) methyl)-3-methoxytetrahydrofuran-2-yl)pyrimidine-2,4(1H,3H)-dione (38.0 g, 78.1 mmol) in DMF (100 mL) was treated with DBU (23.8 g, 156 mmol). Benzyl chloromethyl ether (18.3 g, 117 mmol) was added dropwise at room temperature under nitrogen atmosphere. The resulting mixture was stirred for 2 hours at room temperature under argon atmosphere. The reaction was quenched by NaHCO₃ (sat., 100 mL) at room temperature. The resulting mixture was extracted with EtOAc (200 mL×2). The combined organic layers were washed with H₂O (100 mL) and brine (100 mL), dried over anhydrous Na₂SO₄, filtered and concentrated to afford the titled compound (70 g, 100% yield). MS(ESI): m/z=629.4[M+Na]⁺.

### Step 6: 3-((benzyloxy)methyl)-1-((2R,3R,4R,5R)-4-hydroxy-5-(hydroxymethyl)-3-methoxytetra hydrofuran-2-yl)pyrimidine-2,4(1H,3H)-dione

A solution of 3-((benzyloxy)methyl)-1-((2R,3R,4R,5R)-4-((tert-butyldimethylsilyl)oxy)-5-(((tert-butyldi-methylsilyl)oxy)methyl)-3-methoxytetrahydrofuran-2-yl)pyrimidine-2,4(1H,3H)-dione (70 g, 116 mmol) in THF was treated with trifluoracetic acid (168 mL) and H₂O (168 mL, 9.33 mol) for 2 hours at room temperature. The mixture was neutralized to pH>7 with ammonium hydroxide. The resulting mixture was extracted with EtOAc (500 mL×2). The combined organic layers were washed with H₂O (200 mL), brine (200 mL), dried over anhydrous Na₂SO₄, filtered and concentrated. The residue was purified by silica gel column chromatography, eluted with CH₂Cl₂ and ethyl acetate (1:1, v/v) to afford the titled compound (16.0 g, 36.7% yield) as a white solid. MS(ESI): m/z=401.2 [M+Na]⁺.

### Step 7: 3-((benzyloxy)methyl)-1-((2R,3R,4R,5R)-5-((bis(4-methoxyphenyl)(phenyl)methoxy) methyl)-4-hydroxy-3-methoxytetrahydrofuran-2-yl)pyrimidine-2,4(1H,3H)-dione

A solution of 3-((benzyloxy)methyl)-1-((2R,3R,4R,5R)-4-hydroxy-5-(hydroxymethyl)-3-methoxy-tetrahydrofuran-2-yl)pyrimidine-2,4(1H,3H)-dione (10.0 g, 26.4 mmol) in pyridine (100 mL) was treated with 4,4′-dimethoxytrityl chloride (10.75 g, 31.71 mmol) for overnight at room temperature under nitrogen atmosphere. The reaction was quenched with MeOH (10 mL) at room temperature. The resulting mixture was concentrated under vacuum. The resulting mixture was diluted with EtOAc (800 mL). The resulting mixture was washed with water (100 mL). The organic layer was separated, dried with Na₂SO₄, filtered and concentrated. The residue was purified by silica gel column chromatography, eluted with 0.5% TEA in PE/EA (1:1) to afford the titled compound (17.0 g, 94.5% yield) as a light-yellow solid. MS (ESI): m/z=703.3 [M+Na]⁺.

### Step 8: 3-((benzyloxy)methyl)-1-((2R,3R,4R,5R)-5-((bis(4-methoxyphenyl)(phenyl) methoxy)-methyl)-4-(2-((tert-butyldimethylsilyl)oxy)ethoxy)-3-methoxytetrahydrofuran-2-yl)pyrimidine-2,4(1H,3H)-dione

To a solution of 3-((benzyloxy)methyl)-1-((2R,3R,4R,5R)-5-((bis(4-methoxyphenyl)(phenyl)-methoxy)methyl)-4-hydroxy-3-methoxytetrahydrofuran-2-yl)pyrimidine-2,4(1H,3H)-dione (16.5 g, 24.2 mmol) in DMF (100 mL) was added sodium hydride (60% in oil, 1.93 g, 48.4 mmol) at 0° C. The mixture was stirred for 30 minutes. tert-Butyl(2-iodoethoxy)dimethylsilane (10.4 g, 36.4 mmol) was added and the mixture was allowed to warm to room temperature and stirred for 3 hours. The reaction was quenched with sat. NH₄Cl (aq., 20 mL) at 0° C. The resulting mixture was extracted with ethyl acetate (100 mL×2). The combined organic layers were washed with brine (50 mL), dried over anhydrous Na₂SO₄, filtered and concentrated to afford the titled compound (16 g, 80% yield) as an off-white crude solid. MS(ESI) m/z=861.6 [M+Na]⁺.

### Step 9: 3-((benzyloxy)methyl)-1-((2R,3R,4R,5R)-5-((bis(4-methoxyphenyl)(phenyl)methoxy)-methyl)-4-(2-hydroxyethoxy)-3-methoxytetrahydrofuran-2-yl)pyrimidine-2,4(1H,3H)-dione

A mixture of 3-((benzyloxy)methyl)-1-((2R,3R,4R,5R)-5-((bis(4-methoxyphenyl)(phenyl)-methoxy)-methyl)-4-(2-((tert-butyldimethylsilyl)oxy)ethoxy)-3-methoxytetrahydrofuran-2-yl) pyrimidine-2,4(1H,3H)-dione (16.0 g, 19.1 mmol) and CsF (5.79 g, 38.1 mmol) in DMSO (160 mL) was stirred for overnight at room temperature under air atmosphere. The reaction was quenched with sat. NaHCO₃ (aq., 100 mL) at 0° C. The resulting mixture was extracted with EtOAc (3×300 mL). The combined organic layers were washed with water (100 mL), brine (100 mL), dried over anhydrous Na₂SO₄, filtered and concentrated under reduced pressure. The residue was purified by silica gel column chromatography, eluted with 0.5% TEA in PE/EA (1:2, v/v) to afford the titled compound (4.7 g, 34% yield) as a white solid.

### Step 10: 2-(((2R,3R,4R,5R)-5-(3-((benzyloxy)methyl)-2,4-dioxo-3,4-dihydropyrimidin-1(2H)-yl)-2-((bis(4-methoxyphenyl)(phenyl)methoxy)methyl)-4-methoxytetrahydrofuran-3-yl)oxy)-acetaldehyde

To a stirred solution of 3-((benzyloxy)methyl)-1-((2R,3R,4R,5R)-5-((bis(4-methoxyphenyl) (phenyl)methoxy)-methyl)-4-(2-hydroxyethoxy)-3-methoxytetrahydrofuran-2-yl)pyrimidine-2,4(1H,3H)-dione (4.0 g, 5.5 mmol) in DCM (80 mL) was added Dess-Martin periodinane (2.57 g, 6.07 mmol) at 0° C. The resulting mixture was stirred overnight at room temperature under nitrogen atmosphere. The resulting mixture was diluted with ethyl acetate (400 mL). The combined organic layers were washed with sat. NaHCO₃ (aq., 3×200 mL), brine (2×200 mL), dried over anhydrous Na₂SO₄, filtered and concentrated under reduced pressure to afford the titled compound (4.5 g, 100% yield) as a yellow solid. The crude product was used in the next step directly without further purification. MS(ESI): m/z=763.3 [M+MeCN]⁺.

### Step 11: 3-((benzyloxy)methyl)-1-((2R,3R,4R,5R)-5-((bis(4-methoxyphenyl)(phenyl)methoxy)methyl)-4-(2-((((2R,3R,4R,5R)-3-((tert-butyldimethylsilyl)oxy)-5-(2,4-dioxo-3,4-dihydropyrimidin-1(2H)-yl)-4-methoxy-tetrahydrofuran-2-yl)methyl)(methyl)amino)ethoxy)-3-methoxy-tetrahydrofuran-2-yl)pyrimidine-2,4(1H,3H)-dione

A solution of 2-(((2R,3R,4R,5R)-5-(3-((benzyloxy)methyl)-2,4-dioxo-3,4-dihydropyrimidin-1(2H)-yl)-2-((bis(4-methoxyphenyl)(phenyl)methoxy)methyl)-4-methoxytetrahydrofuran-3-yl)oxy)acetaldehyde (4.0 g, 5.534 mmol) in DCM (40 mL) was treated with 1-((2R,3R,4R,5R)-4-((tert-butyldimethylsilyl)oxy)-3-methoxy-5-((methylamino)methyl)tetrahydrofuran-2-yl)pyrimidine-2,4(1H,3H)-dione (2.13 g, 5.53 mmol) and NaBH(OAc)₃ (2.35 g, 11.1 mmol) for 2 hours at room temperature under nitrogen atmosphere. The reaction was quenched with water (50 mL) at room temperature. The aqueous layer was extracted with EtOAc (3×200 mL). The combined organic layer was concentrated under vacuum. The residue was purified by reversed-phase flash chromatography with the following conditions: column, C18 silica gel; mobile phase, MeCN in water, 5 to 95% gradient in 30 min; detector, UV 254 nm to obtain the titled compound (3.7 g, 61% yield) as a light-yellow solid. MS(ESI): m/z=1092.7 [M+H]⁺.

### Step 12: 1-((2R,3R,4R,5R)-5-(((2-(((2R,3R,4R,5R)-5-(2,4-dioxo-3,4-dihydropyrimidin-1(2H)-yl)-2-(hydroxymethyl)-4-methoxytetrahydrofuran-3-yl)oxy)ethyl)(methyl)amino)methyl)-4-hydroxy-3-methoxytetrahydrofuran-2-yl)pyrimidine-2,4(1H,3H)-dione

Suspended 3-((benzyloxy)methyl)-1-((2R,3R,4R,5R)-5-((bis(4-methoxyphenyl)(phenyl)methoxy) methyl)-4-(2-((((2R,3R,4R,5R)-3-((tert-butyldimethylsilyl)oxy)-5-(2,4-dioxo-3,4-dihydro-pyrimidin-1(2H)-yl)-4-methoxytetrahydrofuran-2-yl)methyl)(methyl)amino)ethoxy)-3-methoxy-tetrahydrofuran-2-yl) pyrimidine-2,4(1H,3H)-dione (3.7 g, 3.4 mmol) in TFA (37 ml) and was heated for 1 hour at 80° C. The resulting mixture was concentrated under vacuum. The residue was dissolved in minimum amount of NaHCO₃ and the pH was adjusted to slightly basic (pH=8). The crude mixture was purified by reversed phase flash chromatography with the following conditions: column, C18 silica gel; mobile phase, MeCN in Water, 0% hold 8 min, then 0 to 95% gradient in 25 min; detector, UV 254 nm. to afford the titled compound (1.5 g, 80% yield) as a light-yellow solid. The product was dried in vacuum oven overnight at room temperature before used in the next step.

### Step 13: 1-((2R,3R,4R,5R)-5-((bis(4-methoxyphenyl)(phenyl) methoxy)methyl)-4-(2-((((2R,3R, 4R,5R)-5-(2,4-dioxo-3,4-dihydropyrimidin-1(2H)-yl)-3-hydroxy-4-methoxytetrahydrofuran-2-yl)methyl)(methyl) amino)ethoxy)-3-methoxytetrahydrofuran-2-yl)pyrimidine-2,4(1H,3H)-dione

To a stirred solution of 1-((2R,3R,4R,5R)-5-(((2-(((2R,3R,4R,5R)-5-(2,4-dioxo-3,4-dihydropyrimidin-1(2H)-yl)-2-(hydroxymethyl)-4-methoxytetrahydrofuran-3-yl)oxy)ethyl)-(methyl)amino)methyl)-4-hydroxy-3-methoxytetrahydrofuran-2-yl)pyrimidine-2,4(1H,3H)-dione (1.7 g, 3.1 mmol) in pyridine (17 mL) were added TEA (0.46 g, 4.590 mmol) and 4,4′-dimethoxytrityl chloride (1.35 g, 3.978 mmol) batchwise at 0° C. The resulting mixture was stirred for 3 hours at room temperature under argon atmosphere. The reaction was quenched by the addition of MeOH (5 mL) at room temperature. The resulting mixture was concentrated under reduced pressure. The residue was purified by reversed-phase flash chromatography with the following conditions: column, C18 silica gel; mobile phase, MeCN in Water, 5 to 95% gradient in 20 min; detector, UV 254 nm to afford the product (2.40 g, 90.5% yield) as an off-white solid. MS(ESI) m/z=858.3 [M+H]⁺.

### Step 14: (2R,3R,4R,5R)-2-(((2-(((2R,3R,4R,5R)-2-((bis(4-methoxyphenyl)(phenyl)methoxy) methyl)-5-(2,4-dioxo-3,4-dihydropyrimidin-1(2H)-yl)-4-methoxytetrahydrofuran-3-yl)oxy)ethyl) (methyl)amino)methyl)-5-(2,4-dioxo-3,4-dihydropyrimidin-1(2H)-yl)-4-methoxytetrahydrofuran-3-yl (2-cyanoethyl) diisopropylphosphoramidite

Cyanoethyl N,N-diisopropylchlorophosphoramidite (1.54 mL, 6.5 mmol) was added to a solution of 1-((2R,3R,4R,5R)-5-((bis(4-methoxyphenyl)(phenyl)methoxy)methyl)-4-(2-((((2R,3R, 4R,5R)-5-(2,4-dioxo-3,4-dihydropyrimidin-1(2H)-yl)-3-hydroxy-4-methoxytetrahydrofuran-2-yl)methyl) (methyl)amino) ethoxy)-3-methoxytetrahydrofuran-2-yl)pyrimidine-2,4(1H,3H)-dione (1.8 g, 2.17 mmol) and DIPEA (2.4 mL, 13 mmol) in DCM (15 mL) dropwise at room temperature. The reaction was stirred for 3 hours. The reaction was quenched by pouring the mixture into an aqueous solution of NaHCO₃ (10 mL) and extracted with DCM (500 mL). The organic layer was separated, dried with Na₂SO₄, filtered and concentrated. The crude was purified by flash column on silica gel column (column with pretreated with 1% Et₃N with hexane) with 0 to 100% EtOAc/Hexane to obtain the product contains the impurities related to the phosphorous reagent. Dissolved the solid with DCM. Added hexane and precipitate was crushed out. The solid was filtered, washed with hexane and ether, and dried in high vacuum to afford the titled compound NB-186 (970 mg, 46% yield). 31P NMR (202 MHz, CDCl₃) δ 150.44, 150.07. MS (ESI): 1058.0 [M+H]⁺, 1080.1 [M+Na]⁺.

Oligonucleotide Comprising Dinucleotide NB-186 has been Synthesized Using the General Procedure Described in Example 1.

### Example 77: NB-187

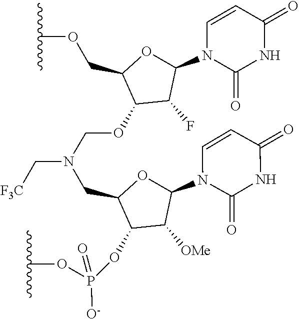 [SMILES: *OC[C@H]1O[C@@H](n2ccc(=O)[nH]c2=O)[C@H](F)[C@@H]1OCN(C[C@H]1O[C@@H](n2ccc(=O)[nH]c2=O)[C@H](OC)[C@@H]1OP(=O)([O-])O*)CC(F)(F)F]

### Synthesis of NB-187: (2R,3R,4R,5R)-2-(((2-((2S,3R,4R,5R)-2-((bis(4-methoxyphenyl)(phenyl)methoxy)methyl)-5-(2,4-dioxo-3,4-dihydropyrimidin-1(2H)-yl)-4-fluorotetrahydrofuran-3-yl)ethyl)(2,2,2-trifluoro-ethyl)amino)methyl)-5-(2,4-dioxo-3,4-dihydropyrimidin-1(2H)-yl)-4-methoxytetrahydrofuran-3-yl (2-cyanoethyl) diisopropylphosphoramidite

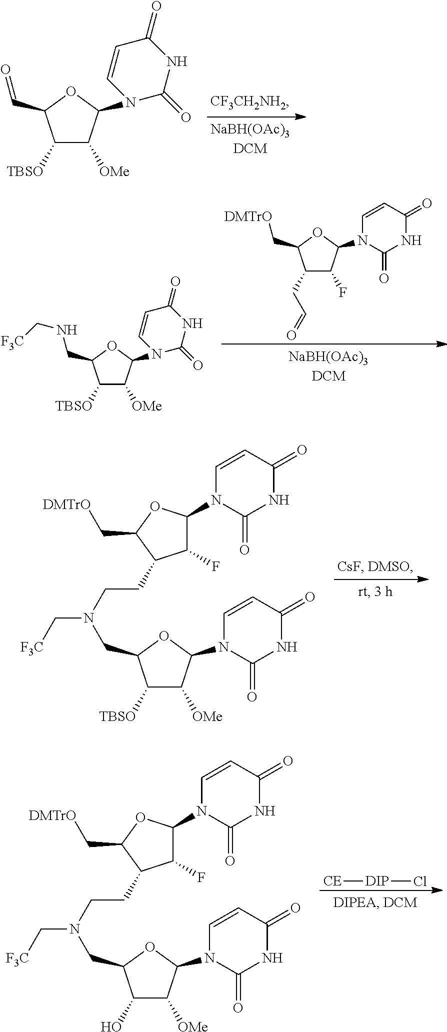

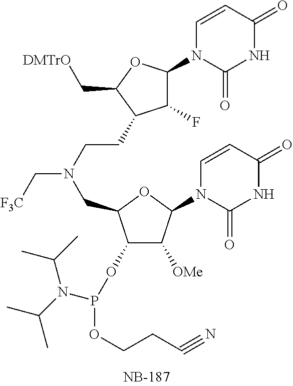

### Step 1: 1-((2R,3R,4R,5R)-4-((tert-butyldimethylsilyl)oxy)-3-methoxy-5-(((2,2,2-trifluoroethyl) amino)methyl)tetrahydrofuran-2-yl)pyrimidine-2,4(1H,3H)-dione

A solution of (2S,3S,4R,5R)-3-[(tert-butyldimethylsilyl)oxy]-5-(2,4-dioxo-3H-pyrimidin-1-yl)-4-methoxy oxolane-2-carbaldehyde (2.4 g, 6.5 mmol) in DCM (240 mL) was treated with 2,2,2-trifluoroethan-1-amine (2.57 g, 25.9 mmol) and NaBH(OAc)₃ (2.75 g, 13.0 mmol), stirred for 2 hours at RT under nitrogen atmosphere. The reaction was quenched with water (10 mL) at room temperature. The aqueous layer was extracted with EtOAc (3×200 mL). The combined organic layers were concentrated under vacuum. The residue was purified by silica gel column chromatography, eluted with PE/EA (1:1) to afford titled compound (1.6 g, 54% yield) as a light yellow oil. MS(ESI): m/z=454.1 [M+H]⁺.

### Step 2: 1-((2R,3R,4R,5S)-5-((bis(4-methoxyphenyl)(phenyl)methoxy)methyl)-4-(2-((((2R,3R,4R,5R)-3-((tert-butyldimethylsilyl)oxy)-5-(2,4-dioxo-3,4-dihydropyrimidin-1(2H)-yl)-4-methoxy tetrahydrofuran-2-yl)methyl)(2,2,2-trifluoroethyl)amino)ethyl)-3-fluorotetrahydro-furan-2-yl)pyrimidine-2,4(1H,3H)-dione

A solution of 1-((2R,3R,4R,5R)-4-((tert-butyldimethylsilyl)oxy)-3-methoxy-5-(((2,2,2-trifluoroethyl) amino) methyl)tetrahydrofuran-2-yl)pyrimidine-2,4(1H,3H)-dione (1.6 g, 3.5 mmol) DCM (16 mL) was added 2-[(2S,3R,4R,5R)-2-([bis(4-methoxyphenyl)(phenyl)methoxy]methyl-5-(2,4-dioxo-3H-pyrimidin-1-yl)-4-fluorooxolan-3-yl]acetaldehyde (4.05 g, 7.06 mmol) and NaBH(OAc)₃ (2.24 g, 10.6 mmol) at RT under nitrogen atmosphere. The resulting mixture was stirred overnight at RT. The resulting mixture was extracted with EA (2×500 mL). The combined organic layers were washed with brine (100 mL), dried over anhydrous Na₂SO₄. After filtration, the filtrate was concentrated under reduced pressure. The residue was purified by silica gel column chromatography, eluted with EA/DCM (contained 0.5% TEA) to afford the titled compound (1.2 g, 34% yield) as a white solid: MS(ESI): m/z=1012.5 [M+H]⁺, and the titled compound with TBS deprotected (1.0 g, 24% yield): MS (ESI) m/z=898.3 [M+H]⁺.

### Step 3: 1-((2R,3R,4R,5S)-5-((bis(4-methoxyphenyl)(phenyl)methoxy)methyl)-4-(2-((((2R,3R,4R,5R)-5-(2,4-dioxo-3,4-dihydropyrimidin-1(2H)-yl)-3-hydroxy-4-methoxytetrahydrofuran-2-yl)methyl) (2,2,2-trifluoroethyl)amino)ethyl)-3-fluorotetrahydrofuran-2-yl)pyrimidine-2,4(1H,3H)-dione

A solution of 1-[(2R,3R,4R,5S)-5-([bis(4-methoxyphenyl)(phenyl)methoxy]methyl-4-[2-(([(2R,3R,4R,5R)-3-[(tert-butyldimethylsilyl)oxy]-5-(2,4-dioxo-3H-pyrimidin-1-yl)-4-methoxy oxolan-2-yl]methyl(2,2,2-trifluoroethyl)amino)ethyl]-3-fluorooxolan-2-yl]-3H-pyrimidine-2,4-dione (1.2 g, 1.2 mmol) and CsF (360 mg, 2.37 mmol) in DMSO (12 mL) was stirred for 1 hour at RT under nitrogen atmosphere. The resulting mixture was extracted with EA (500 mL). The combined organic layers were washed with brine (50 mL), dried over anhydrous Na₂SO₄. After filtration, the filtrate was concentrated under reduced pressure. The residue was purified by flash chromatography with the following conditions: column, C18; mobile phase, ACN in H₂O, 10 to 95% gradient in 30 min; UV 254 nm. to afford the titled compound (784 mg, 71.7% yield) as a white solid. MS (ESI) m/z=898.3 [M+H]⁺.

### Step 4: (2R,3R,4R,5R)-2-(((2-((2S,3R,4R,5R)-2-((bis(4-methoxyphenyl) (phenyl)methoxy)methyl)-5-(2,4-dioxo-3,4-dihydropyrimidin-1(2H)-yl)-4-fluorotetrahydrofuran-3-yl)ethyl)(2,2,2-trifluoroethyl)amino)methyl)-5-(2,4-dioxo-3,4-dihydropyrimidin-1(2H)-yl)-4-methoxytetrahydrofuran-3-yl (2-cyanoethyl) diisopropylphosphoramidite

2-Cyanoethyl N,N-diisopropylchlorophosphoramidite (0.6 mL, 2.54 mmol) was added to a solution of 1-((2R,3R,4R,5S)-5-((bis(4-methoxyphenyl) (phenyl)methoxy)methyl)-4-(2-((((2R,3R,4R,5R)-5-(2,4-dioxo-3,4-dihydropyrimidin-1(2H)-yl)-3-hydroxy-4-methoxytetrahydro furan-2-yl)methyl)(2,2,2-trifluoroethyl) amino)ethyl)-3-fluorotetrahydrofuran-2-yl)pyrimidine-2,4(1H,3H)-dione (720 mg, 0.80 mmol) and DIPEA (1.0 mL, 5.4 mmol) in DCM (15 mL). The reaction was allowed to stir for 3 hours. Poured the reaction mixture into an aqueous solution of NaHCO₃ (10 mL) and extracted with DCM (2×500 mL). The combined organic layers were dried with Na₂SO₄, filtered and concentrated. The crude was purified by flash column on 40 g silica gel column (column with pretreated with 1% Et₃N with hexane) with 0 to 100% EA/H to afford a solid. Titrated the solid with DCM/hexane to obtain the titled compound NB-187 (535 mg, 52% yield) as a white power. 31P NMR (202 MHz, CDCl₃) δ 150.34, 149.81. MS (ESI): m/z=1013.4, [M−N(iPr)₂+OH—H]⁺.

Oligonucleotide Comprising Dinucleotide NB-187 has been Synthesized Using the General Procedure Described in Example 1.

### Example 78: NB-188

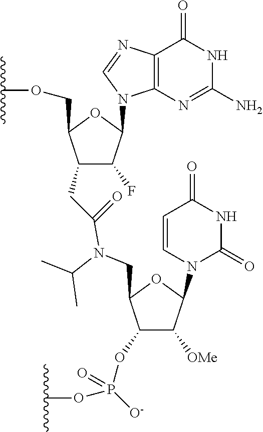 [SMILES: *OC[C@H]1O[C@@H](n2cnc3c(=O)[nH]c(N)nc32)[C@H](F)[C@@H]1CC(=O)N(C[C@H]1O[C@@H](n2ccc(=O)[nH]c2=O)[C@H](OC)[C@@H]1OP(=O)([O-])O*)C(C)C]

### Synthesis of 188

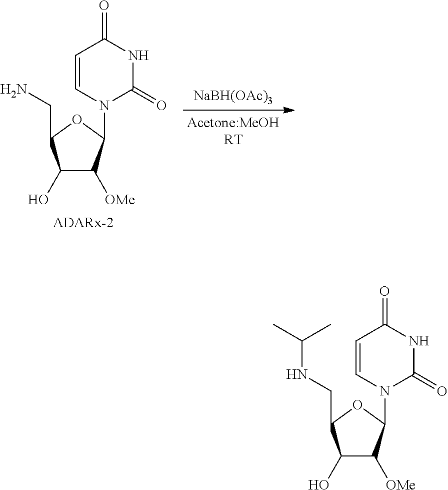

1-((2S,3S,4S,5S)-4-hydroxy-5-((isopropylamino)methyl)-3-methoxytetrahydrofuran-2-yl)pyrimidine-2,4(1H,3H)-dione. To a stirred solution of Amine (3 g, 11.7 mmol, 1 eq) in Acetone:MeOH (80 mL), stirred at RT for 30 min. Then NaBH(OAc)₃ added at RT. After 1 hr. the reaction went to completion and mixture was concentrated. The residue was purified by column chromatography (Biotage 25 g, 20 micron) using 0-30% MeOH/DCM. Then Pure compound was diluted with Sat NaHCO₃, extracted with 3× (10% DCM in MeOH) 300 mL and dried over Na₂SO₄ and concentrated to obtain isopropyl amine 1.83 g 52.3% as white solid. ¹H NMR (500 MHz, d-DMSO): δ 8.0 (d, J=5 Hz, 1H), 5.8 (d, J=5 Hz, 1H), 5.6 (J=5 Hz, 1H), 5.1 (br, 1H), 4.1 (br, 1H), 3.8 (t, J=5 Hz, 1H), 5.6-5.5 (m, H), 3.3 (s, 3H), 2.7 (m, 3H), 1.0 (m, 6H). Mass (ESI) m/z: [M+H]⁺ Cacld for 300.33, found: 300.23.

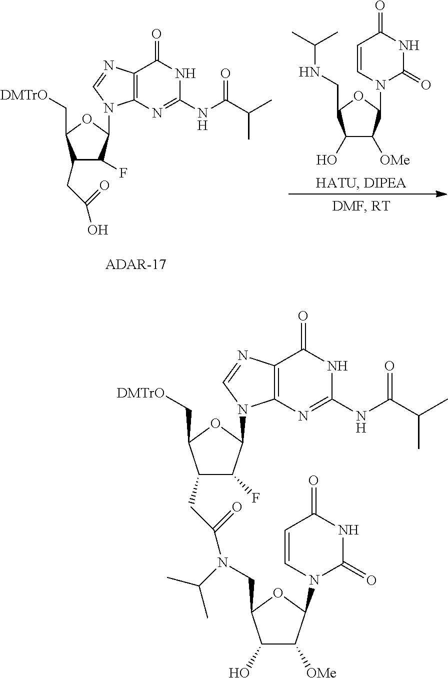

N-(9-((2R,3R,4R,5S)-5-((bis(4-methoxyphenyl)(phenyl)methoxy)methyl)-4-(2-((((2R,3R,4R,5R)-5-(2,4-dioxo-3,4-dihydropyrimidin-1(2H)-yl)-3-hydroxy-4-methoxytetrahydrofuran-2-yl)methyl)(isopropyl)amino)-2-oxoethyl)-3-fluorotetrahydrofuran-2-yl)-6-oxo-6,9-dihydro-1H-purin-2-yl)isobutyramide A solution of acid (3.43 g, 4.9 mmol, 1 eq), amine (1.7 g, 5.88 mmol, 1.2 eq) and HATU (2.92 g, 7.7 mmol, 1.5 eq) in DMF (20 mL) was added DIPEA (2.73 mL, 15.39 mmol, 3 eq), and the mixture was stirred for 1 hr. Then additional 0.2 eq amine (0.3 g, 0.2 eq) added and reaction was stirred overnight. Reaction mixture was added dropwise to a vigorously stirring solution of Aq. saturated NaHCO₃ (60 ml), precipitated solids were filtered and washed with NaCl. purified by flash chromatography (100 g 20 micron Biotage column) using MeOH/EtOAc, 0-20% 5 CV then 20% 10 CV as an eluent, to obtain amide (2.19 g, 45%) as a white solid. ¹H NMR (500 MHz, d-DMSO): δ 12.4-11.4 (br, 3H), 8.0 (d, 1H, J=20 Hz), 7.8 (d, 1H, J=5 Hz), 7.4 (d, 1H, J=5 Hz), 7.4-7.2 (m, 7H), 6.2 (m, 1H), 5.8-5.3 (m, 8H), 4.3-3.8 (m, 2H), 4.5 (m, 1H), 4.3-3.8 (m, 7H), 3.7 (m, 20H), 3.5-3.1 (m, 14H), 2.8-2.5 (m, 14H), 2.6-2.5 (m, 3H), 1.2-1.1 (m, 30H). Mass (ESI) m/z: [M+Na]⁺ Cacld for 1003.4, found: 1003.2.

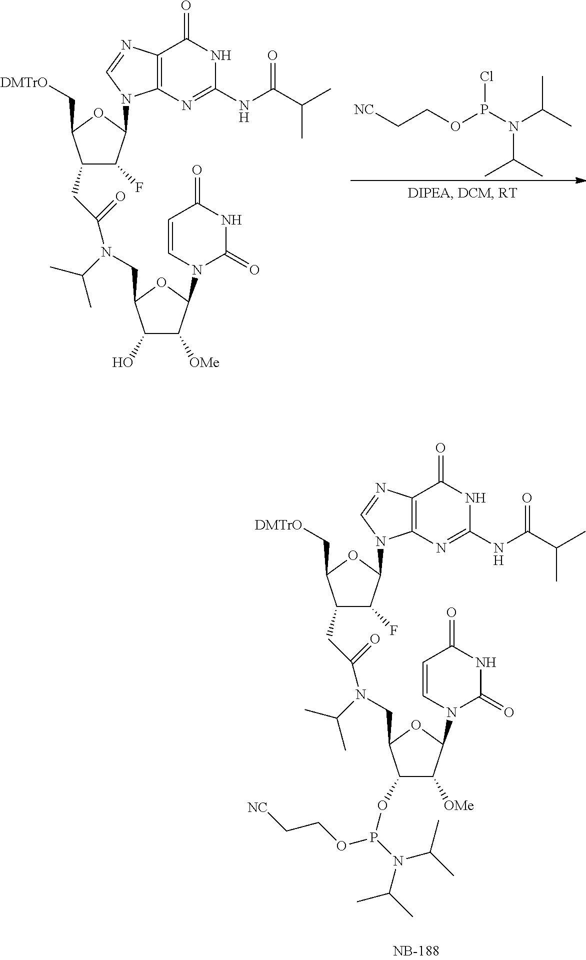

(2R,3R,4R,5R)-2-((2-((2S,3R,4R,5R)-2-((bis(4-methoxyphenyl)(phenyl) methoxy)methyl)-4-fluoro-5-(2-isobutyramido-6-oxo-1,6-dihydro-9H-purin-9-yl)tetrahydrofuran-3-yl)-N-isopropylacetamido)methyl)-5-(2,4-dioxo-3,4-dihydropyrimidin-1(2H)-yl)-4-methoxytetrahydrofuran-3-yl (2-cyanoethyl) diisopropylphosphoramidite. To a stirred solution of alcohol (2.19, 2.230 mmol, 1 eq) and diisopropylethylamine (2.32 mL, 13.4 mmol, 6 eq), in DCM (15 mL), was added N, N-diisopropyl chlorophosphoramidite (1.57 mL 6.7 mmol, 3 eq) dropwise. The reaction mixture was stirred at room temperature for 3 h. reaction mixture was quenched diluted with Aq. Saturated NaHCO₃ solution (100 mL), extracted with DCM (100) ml, washed with brine (100 mL), dried over Na₂SO₄, and evaporated the crude product was loaded on to (pre-equilibrated with 1% Et₃N-EtOAc) Biotage silica gel column (100 g 20 μm), and purified by flash chromatography using MeOH/EtOAc 0-10% 10 CV, 10% 5 CV, containing 1% Et₃N as an additive, gave NB-188, 1.74 g (66%) of pure product. ¹H NMR (500 MHz, d-DMSO): δ 12.4-11.2 (br, 3H), 8.2 (m, 3H), 7.8 (m, 2H), 7.6 (m, 1H), 7.4-7.2 (m, 10H), 6.7 (m, 5H), 6.3 (m, 2H), 5.8-5.4 (m, 4H), 4.5 (m, 1H), 4.3-3.8 (m, 7H), 3.7 (m, 20H), 3.5-3.1 (m, 14H), 2.8-2.5 (m, 14H), 2.6-2.5 (m, 3H), 1.2-1.1 (m, 30H). ³¹P NMR (202, MHz, DMSO-d6) δ 149.50, 149.34, 149.22, 149.18. Mass (ESI) m/z: [M+Na]⁺ Cacld for 1204.27, found: 1204.5

Oligonucleotide Comprising Dinucleotide NB-188 has been Synthesized Using the General Procedure Described in Example 1.

### Example 79: NB-189

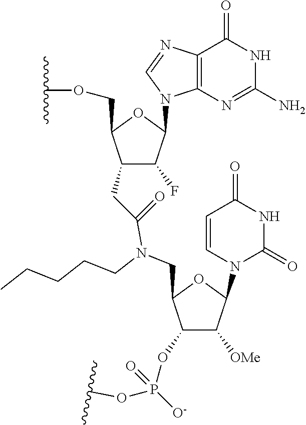 [SMILES: *OC[C@H]1O[C@@H](n2cnc3c(=O)[nH]c(N)nc32)[C@H](F)[C@@H]1CC(=O)N(CCCCC)C[C@H]1O[C@@H](n2ccc(=O)[nH]c2=O)[C@H](OC)[C@@H]1OP(=O)([O-])O*]

### Synthesis of 189

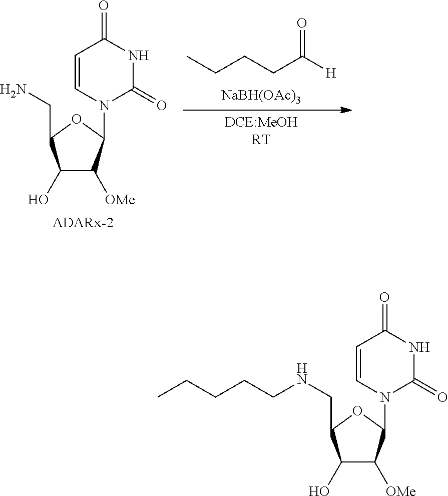

1-((2S,3S,4S,5S)-4-hydroxy-3-methoxy-5-((pentylamino) methyl) tetrahydrofuran-2-yl)pyrimidine-2,4(1H,3H)-dione. A mixture of aldehyde (1.1 g, 11.7 mmol, 1.0 eq), Amine (3.0 g, 11.7, 1 eq) in DCE/MeOH (50 mL/10 mL) was stirred for 30 min at RT. Then NaBH(OAc)₃ added at RT (3.4 g, 16.4 mmol, 1.4 eq) was added and the mixture was stirred for 12 hr at room temperature. Reaction mixture was concentrated. Residue was purified by column chromatography (Biotage 25 g, 20 micron) using 0-30% MeOH/DCM 8 CV, obtained desired product as white solid 1.48 g (45%). ¹H NMR (500 MHz, d-DMSO): δ 7.8 (d, J=5 Hz, 1H), 6.7 (d, J=5 Hz, 1H), 5.6 (J=5 Hz, 1H), 3.8 (t, J=5 Hz, 1H), 3.7 (m, 2H), 3.3 (s, 3H), 2.7 (m, 2H), 2.6 (m, 2H), 2.5 (m, 2H) 1.4 (m, 2H) 1.3 (m, 3H), (0.7, m, 3H). Mass (ESI) m/z: [M+H]⁺ Cacld for 328.38, found: 328.42.

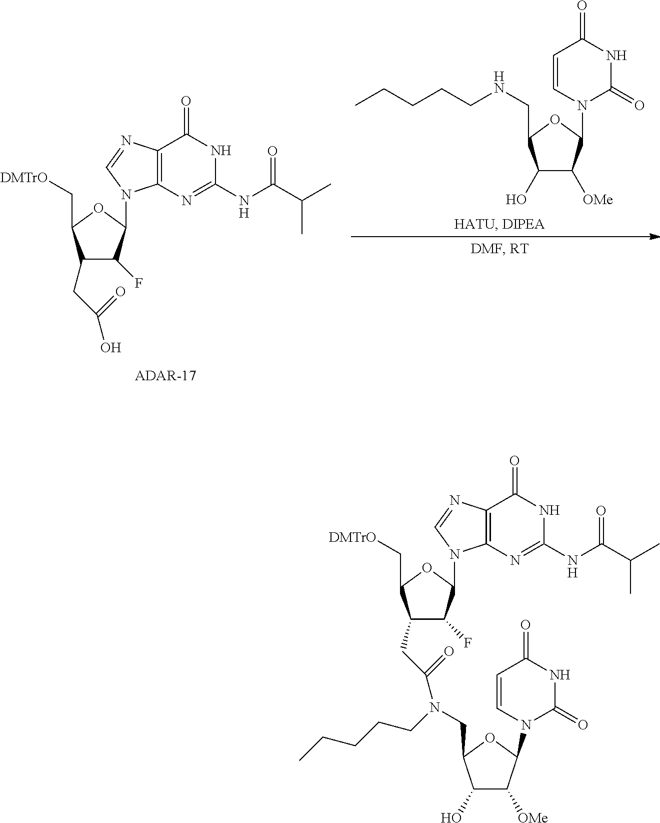

N-(9-((2S,3R,4R,5R)-5-((bis(4-methoxyphenyl)(phenyl)methoxy)methyl)-4-(2-((((2R,3R,4R,5R)-5-(2,4-dioxo-3,4-dihydropyrimidin-1(2H)-yl)-3-hydroxy-4-methoxytetrahydrofuran-2-yl)methyl)(pentyl)amino)-2-oxoethyl)-3-fluorotetrahydrofuran-2-yl)-6-oxo-6,9-dihydro-1H-purin-2-yl)isobutyramide A solution of acid (3.5 g, 5.1 mmol, 1 eq), amine (2 g, 6.11 mmol, 1.3 eq) and HATU (2.9 g, 7.7 mmol, 1.5 eq) in DMF (23 mL) was added DIPEA (2.7, 15.3 mmol, 3 eq) at RT), and the mixture was stirred for 1 hr 30 min at room temperature. Reaction mixture was added to a stirring solution of sat. NaHCO₃, precipitated solids were filtered and washed with water and dried under high vac overnight Purified by flash chromatography (25 g 20 micron Biotage column) using MeOH/EtOAc, 0-20% 5 CV then 20% 10 CV as an eluent, to obtain amide (3.01 g, 58.4%) as a beige solid. LCMS m/z 980.05 (M⁺−1) are corresponding with the product.

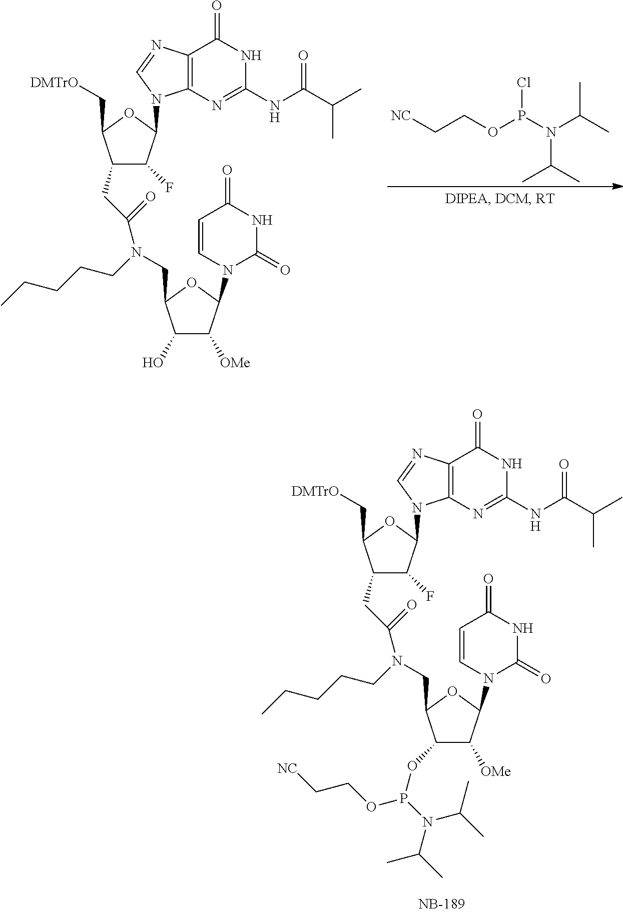

(2R,3R,4R,5R)-2-((2-((2S,3R,4R,5R)-2-((bis(4-methoxyphenyl) (phenyl)methoxy)methyl)-4-fluoro-5-(2-isobutyramido-6-oxo-1,6-dihydro-9H-purin-9-yl)tetrahydrofuran-3-yl)-N-pentylacetamido)methyl)-5-(2,4-dioxo-3,4-dihydropyrimidin-1(2H)-yl)-4-methoxytetrahydrofuran-3-yl (2-cyanoethyl) diisopropylphosphoramidite. To a stirred solution of alcohol (2.8 g, 2.77 mmol, 1 eq) and diisopropylethylamine (2.9 mL, 16.6 mmol, 6 eq), in DCM (18 mL), was added N, N-diisopropyl chlorophosphoramidite (2 mL 8.32 mmol, 3 eq) dropwise. The reaction mixture was stirred at room temperature for 3 h. reaction mixture was quenched diluted with Aq. Saturated NaHCO₃ solution (100 mL), extracted with DCM (100) ml, washed with brine (100 mL), dried over Na₂SO₄, and evaporated the crude product was loaded on to (pre-equilibrated with 1% Et₃N-EtOAc) Biotage silica gel column (100 g 20 μm), and purified by flash chromatography using MeOH/EtOAc 0-10% 10 CV, 10% 5 CV, containing 1% Et₃N as an additive, gave 1.74 g (53%) of NB-189. ¹H NMR (500 MHz, d-DMSO): δ 12.4-11.0 (br, 3H), 8.0 (m, 2H), 7.8-7.6 (m, 2H), 7.4-7.1 (m, 9H), 7.4-7.2 (m, 10H), 6.8 (m, 4H), 6.3 (m, 1H), 5.8-5.4 (m, 3H), 4.5 (m, 1H), 4.3-4.0 (m, 5H), 3.9-3.5 (m, 15H), 3.5-3.1 (m, 10H), 2.8-2.5 (m, 14H), 2.6-2.5 (m, 2H), 1.2-1.1 (m, 20H). ³¹P NMR (202, MHz, DMSO-d6) δ 149.90, 149.66, 149.46, 149.31. Mass (ESI) m/z: [M+Na]⁺ Cacld for 1232.27, found: 1232.1

Oligonucleotide Comprising Dinucleotide NB-189 has been Synthesized Using the General Procedure Described in Example 1.

### Example 80: NB-190

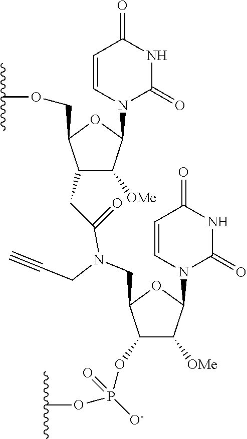

### Synthesis of NB-190

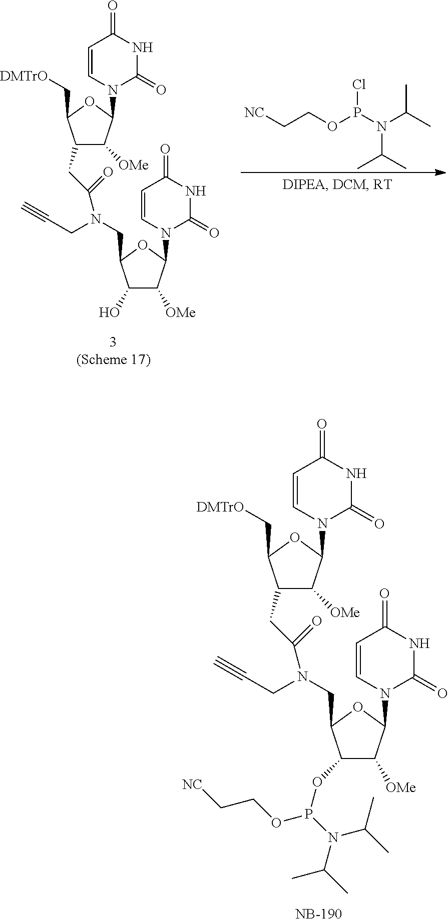

To a stirred solution of alcohol 3 (2 g, 2.275 mmol, 1 eq) and diisopropylethylamine (2.37 mL, 13.65 mmol, 6 eq), in DCM (20 mL), was added N, N-diisopropyl chlorophosphoramidite (1.5 mL 6.8 mmol, 3 eq) dropwise. The reaction mixture was stirred at room temperature for 5 h. Reaction mixture was quenched with Aq. Saturated NaHCO₃ solution (100 mL), extracted with DCM (200) ml, washed with brine (100 mL), dried over Na₂SO₄, and evaporated the crude product was loaded on to (pre-equilibrated with 1% Et₃N-EtOAc) Biotage silica gel column (100 g 20 μm), and purified by flash chromatography using MeOH/EtOAc 0-5% 5 CV, 5% 10 CV, containing 1% Et₃N as an additive, gave about 1 g pure product and 2 g mixture with reagent. The mixture was concentrated and re dissolved in DCM 50 mL then 50 ml of Hexane was added slowly to get the precipitation, then the hexane was decanted, resulting gum was dissolved in DCM and combined with pure product and dried under high vacuum to obtain Phosphoramidite NB-190, 1.83 g, 73% as a white solid. 95% purity by LCMS, HPLC and P31, Mass (m/z 1102 (M++Na). ³¹P NMR (202, MHz, DMSO-d6) δ 149.92, 149.68, 149.54.

### Oligonucleotide Comprising Dinucleotide NB-190 to be Synthesized Using the General Procedure Described in Example 1.

### Example 81: NB-192

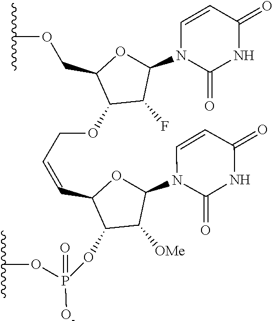

### Synthesis of NB-192: (2R,3R,4R,5R)-2-((Z)-3-(((2R,3R,4R,5R)-2-((bis(4-methoxyphenyl)(phenyl)methoxy)methyl)-5-(2,4-dioxo-3,4-dihydropyrimidin-1(2H)-yl)-4-fluorotetrahydrofuran-3-yl)oxy)prop-1-en-1-yl)-5-(2,4-dioxo-3,4-dihydropyrimidin-1(2H)-yl)-4-methoxytetrahydrofuran-3-yl(2-cyano-ethyl) diisopropylphosphoramidite

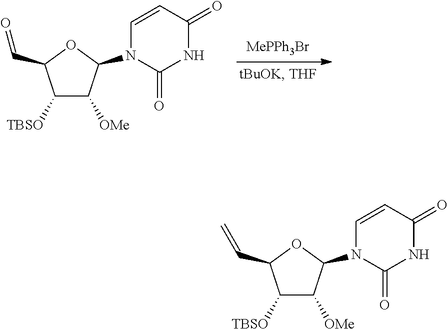

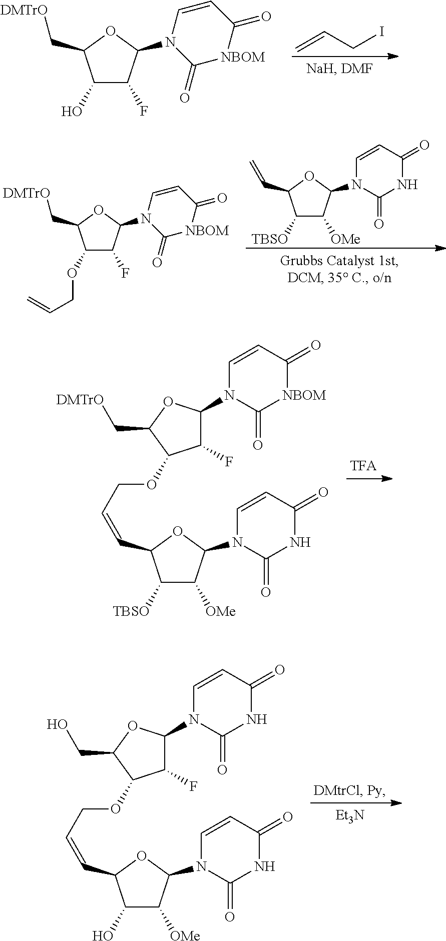

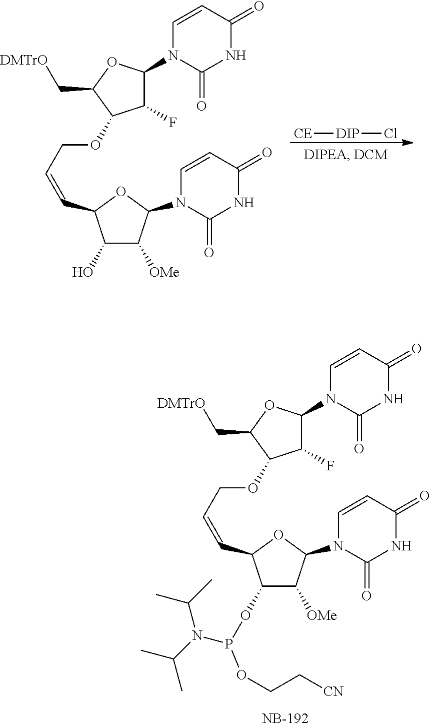

### Step 1: 1-((2R,3R,4R,5R)-4-((tert-butyldimethylsilyl)oxy)-3-methoxy-5-vinyltetrahydrofuran-2-yl)pyrimi-dine-2,4(1H,3H)-dione

To a stirred solution of methyltriphenylphosphonium bromide (90.93 g, 254.5 mmol) in THF (450 mL) were added potassium tert-butoxide (1.8 M in THF, 138 mL, 248 mmol) dropwise at 0° C. under Ar atmosphere. The resulting mixture was stirred for 1 hour at 0° C. To the above mixture was added (2S,3S,4R,5R)-3-((tert-butyldimethylsilyl)oxy)-5-(2,4-dioxo-3,4-dihydropyrimidin-1(2H)-yl)-4-methoxy-tetrahydrofuran-2-carbaldehyde (46.0 g, 124 mmol) in THF (150 mL) dropwise at 0° C. The resulting mixture was stirred for 3 hours at room temperature. The reaction was quenched by saturated NH4Cl solution (500 mL). The reaction mixture was diluted with EA (3 L). The organic layer was washed with saturated NH4Cl solution (2×300 mL), brine (2×300 mL) and dried by Na₂SO₄. After filtration, the filtrate was concentrated under reduced pressure. The residue was purified by silica gel column chromatography, eluted with PE:EA (1:1) to afford the titled compound (30 g, 66% yield) as an off-white solid. MS(ESI): m/z=369.2[M+H]⁺.

### Step 2: 1-((2R,3R,4R,5R)-4-(allyloxy)-5-((bis(4-methoxyphenyl)(phenyl)methoxy)methyl)-3-fluorotetra-hydrofuran-2-yl)-3-((benzyloxy)methyl)pyrimidine-2,4(1H,3H)-dione

To a stirred solution of 3-((benzyloxy)methyl)-1-((2R,3R,4R,5R)-5-((bis(4-methoxyphenyl) (phenyl) methoxy)methyl)-3-fluoro-4-hydroxytetrahydrofuran-2-yl)pyrimidine-2,4(1H,3H)-dione (20.0 g, 29.9 mmol) in DMF (100 mL) were added NaH (2.39 g, 59.8 mmol, 60% in mineral oil) portionwise at 0° C. under N₂ atmosphere. The resulting mixture was stirred for 5 minutes at 0° C. To the above mixture was added 3-iodoprop-1-ene (7.54 g, 44.9 mmol) in portions at 0° C. The resulting mixture was stirred for an additional 1 hour at RT. The reaction was quenched by cold water (500 mL) at room temperature. The resulting mixture was extracted with EA (3×500 mL). The combined organic layers were washed with water (2×500 mL) and brine (100 mL), dried by Na₂SO₄.

After filtration, the filtrate was concentrated under reduced pressure. The crude material was purified by silica gel column and eluted with 1:1 PE:EA) to afford the titled compound (19.9 g, 93.9% yield) as a yellow oil. MS(ESI): m/z=731.2 [M+Na]⁺.

### Step 3: 3-((benzyloxy)methyl)-1-((2R,3R,4R,5R)-5-((bis(4-methoxyphenyl)(phenyl)methoxy) methyl)-4-(((Z)-3-((2R,3R,4R,5R)-3-((tert-butyldimethylsilyl)oxy)-5-(2,4-dioxo-3,4-dihydro pyrimidin-1(2H)-yl)-4-methoxytetrahydrofuran-2-yl)allyl)oxy)-3-fluorotetrahydrofuran-2-yl)pyrimidine-2,4(1H,3H)-dione

To a stirred solution of 1-((2R,3R,4R,5R)-4-(allyloxy)-5-((bis(4-methoxyphenyl)(phenyl)methoxy) methyl)-3-fluorotetrahydrofuran-2-yl)-3-((benzyloxy)methyl) pyrimidine-2,4(1H,3H)-dione (10.0 g, 14.1 mmol) and 1-((2R,3R,4R,5R)-4-((tert-butyl dimethyl silyl)oxy)-3-methoxy-5-vinyltetrahydro furan-2-yl)pyrimidine-2,4(1H,3H)-dione (5.20 g, 14.1 mmol) in DCM (50 mL) were added benzylidene-bis(tricyclohexyl-phosphine)dichlororuthenium (2.95 g, 3.53 mmol) in portions at RT under N2 atmosphere. The resulting mixture was stirred overnight at 35° C. under N2 atmosphere. After filtration, the filtrate was concentrated under reduced pressure. The residue was purified by silica gel column chromatography (eluted with PE:EA (1:1)) to afford the titled compound (4.5 g, 30% yield) as a green solid. MS (ESI): m/z=1071.3 [M+Na]⁺.

### Step 4: 1-((2R,3R,4R,5R)-4-(((Z)-3-((2R,3R,4R,5R)-5-(2,4-dioxo-3,4-dihydropyrimidin-1(2H)-yl)-3-hydroxy-4-methoxytetrahydrofuran-2-yl)allyl)oxy)-3-fluoro-5-(hydroxymethyl)tetrahydrofuran-2-yl)pyrimidine-2,4(1H,3H)-dione

To a stirred solution of 3-((benzyloxy)methyl)-1-((2R,3R,4R,5R)-5-((bis(4-methoxyphenyl)(phenyl) methoxy)methyl)-4-(((Z)-3-((2R,3R,4R,5R)-3-((tert-butyldimethylsilyl)oxy)-5-(2,4-dioxo-3,4-dihydro pyrimidin-1(2H)-yl)-4-methoxytetrahydrofuran-2-yl)allyl)oxy)-3-fluorotetrahydro-furan-2-yl)pyrimidine-2,4(1H,3H)-dione (2.0 g, 1.9 mmol) in TFA (12 mL) at room temperature under N2 atmosphere. The resulting mixture was stirred for 30 minutes at 80° C. under N2 atmosphere. The resulting mixture was concentrated under reduced pressure. The reaction was quenched by the aqueous saturated NaHCO₃ solution (50 mL) at room temperature and adjusted the pH of the solution to 8. The mixture was extracted with DCM (200 mL×2), dried with Na₂SO₄, filtered and concentrated. The residue was purified with reversed-phase flash chromatography with the following conditions: column, C18 silica; mobile phase, CH3CN in water, 10 to 90% gradient in 20 min; detector, UV 254 nm, to afford the titled compound (500 mg, 50% yield) as a light-yellow solid. MS (ESI): m/z=513.2 [M+H]⁺, 535.0 [M+Na]⁺.

### Step 5: 1-((2R,3R,4R,5R)-5-((bis(4-methoxyphenyl)(phenyl)methoxy)methyl)-4-(((Z)-3-((2R,3R,4R,5R)-5-(2,4-dioxo-3,4-dihydropyrimidin-1(2H)-yl)-3-hydroxy-4-methoxytetrahydro furan-2-yl)allyl)oxy)-3-fluorotetrahydrofuran-2-yl)pyrimidine-2,4(1H,3H)-dione

To a stirred solution of 1-((2R,3R,4R,5R)-4-(((Z)-3-((2R,3R,4R,5R)-5-(2,4-dioxo-3,4-dihydropyrimidin-1(2H)-yl)-3-hydroxy-4-methoxytetrahydrofuran-2-yl)allyl)oxy)-3-fluoro-5-(hydroxymethyl)tetrahydrofuran-2-yl)pyrimidine-2,4(1H,3H)-dione (500 mg, 0.976 mmol, pre-dried by vacuum oven) in pyridine (5 mL) were added 4,4′-dimethoxytriphenylmethyl chloride (430 mg, 1.27 mmol) and triethylamine (0.20 mL, 1.5 mmol) dropwise at room temperature at N2 atmosphere. The resulting mixture was stirred overnight at room temperature under N2 atmosphere. The reaction was quenched by water (100 mL) at room temperature. The resulting mixture was extracted with EA (3×100 mL). The combined organic layers were washed with water (2×50 mL) and brine (50 mL), dried by Na₂SO₄. After filtration, the filtrate was concentrated under reduced pressure. The residue was purified by reversed-phase flash chromatography with the following conditions: column, C18 silica gel; mobile phase, ACN in water, 10% to 90% gradient in 20 min; detector, UV 254 nm to afford the titled compound (521 mg, 65% yield) as a brown solid. MS(ESI, negative mode): m/z=813.20 [M−H]+.

Step 6: (2R,3R,4R,5R)-2-((Z)-3-(((2R,3R,4R,5R)-2-((bis(4-methoxyphenyl)(phenyl)methoxy)methyl)-5-(2,4-dioxo-3,4-dihydropyrimidin-1(2H)-yl)-4-fluorotetrahydrofuran-3-yl)oxy)prop-1-en-1-yl)-5-(2,4-dioxo-3,4-dihydropyrimidin-1(2H)-yl)-4-methoxytetrahydrofuran-3-yl (2-cyanoethyl) diisopropylphosphoramidite 2-Cyanoethyl N,N-diisopropylchlorophosphoramidite (0.5 mL, 2.10 mmol) was added to a solution of 1-((2R,3R,4R,5R)-5-((bis(4-methoxyphenyl)(phenyl)methoxy)methyl)-4-(((Z)-3-((2R, 3R,4R,5R)-5-(2,4-dioxo-3,4-dihydropyrimidin-1(2H)-yl)-3-hydroxy-4-methoxytetrahydrofuran-2-yl)allyl)oxy)-3-fluorotetrahydro furan-2-yl)pyrimidine-2,4(1H,3H)-dione (500 mg, 0.61 mmol) and DIPEA (1 mL, 5.54 mmol) in DCM (5 mL). The reaction was allowed to stir for 2 hours. The reaction was poured into aqueous NaHCO₃ (sat., 50 mL) solution and extracted with DCM (3×100 mL). The combined organic layer was separated, dried with Na₂SO₄, filtered and concentrated. The crude was purified by flash column on 40 g silica gel column (column with pretreated with 1% Et₃N with hexane) with 0 to 100% EA/H to afford the crude product which was titrated with DCM/Et₂O/Hexane to afford the titled compound NB-192 (200 mg, 31% yield) as a white solid. 31P NMR (202 MHz, CDCl3) δ 151.03, 150.34. MS (ESI): m/z=1037.3 [M+Na]⁺.

Oligonucleotide Comprising Dinucleotide NB-192 has been Synthesized Using the General Procedure Described in Example 1.

### Example 82: NB-193

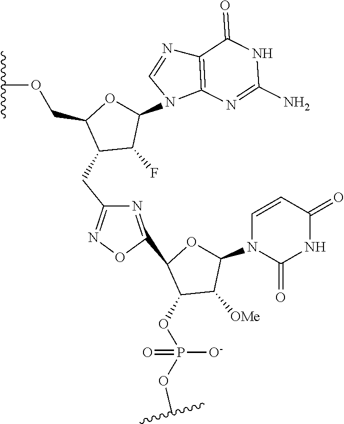 [SMILES: *OC[C@H]1O[C@@H](n2cnc3c(=O)[nH]c(N)nc32)[C@H](F)[C@@H]1Cc1noc([C@H]2O[C@@H](n3ccc(=O)[nH]c3=O)[C@H](OC)[C@@H]2OP(=O)([O-])O*)n1]

### Synthesis of NB-193: (2S,3S,4R,5R)-2-(3-(((2S,3R,4R,5R)-2-((bis(4-methoxyphenyl)(phenyl)methoxy)methyl)-4-fluoro-5-(2-isobutyramido-6-oxo-1,6-dihydro-9H-purin-9-yl)tetrahydrofuran-3-yl)methyl)-1,2,4-oxadiazol-5-yl)-5-(2,4-dioxo-3,4-dihydropyrimidin-1(2H)-yl)-4-methoxytetrahydrofuran-3-yl(2-cyanoethyl) diisopropylphosphoramidite

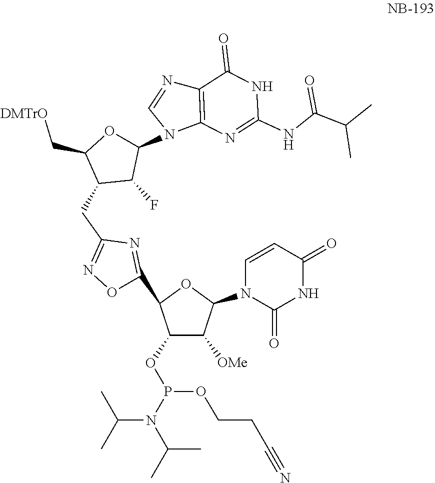

NB-193 was made using the same method as in NB-201 by replacing the acid with 2-((2S,3R,4R,5R)-2-((bis(4-methoxyphenyl)(phenyl)methoxy)methyl)-4-fluoro-5-(2-isobutyramido-6-oxo-1,6-dihydro-9H-purin-9-yl)tetrahydrofuran-3-yl)acetic acid in step 1 to afford the titled compound NB-193 as a white solid. 31P NMR (202 MHz, CDCl₃) δ 152.07, 151.40. MS (ESI): m/z=1172.2 [M+Na]⁺.

Oligonucleotide Comprising Dinucleotide NB-193 has been Synthesized Using the General Procedure Described in Example 1.

### Example 83: NB-194

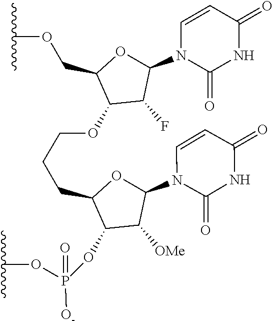

### Synthesis of NB-194: (2R,3R,4R,5R)-2-(3-(((2R,3R,4R,5R)-2-((bis(4-methoxyphenyl)(phenyl) methoxy)methyl)-5-(2,4-dioxo-3,4-dihydropyrimidin-1(2H)-yl)-4-fluorotetrahydrofuran-3-yl)oxy)propyl)-5-(2,4-dioxo-3,4-dihydropyrimidin-1(2H)-yl)-4-methoxytetrahydrofuran-3-yl(2-cyanoethyl) diisopropylphosphoramidite

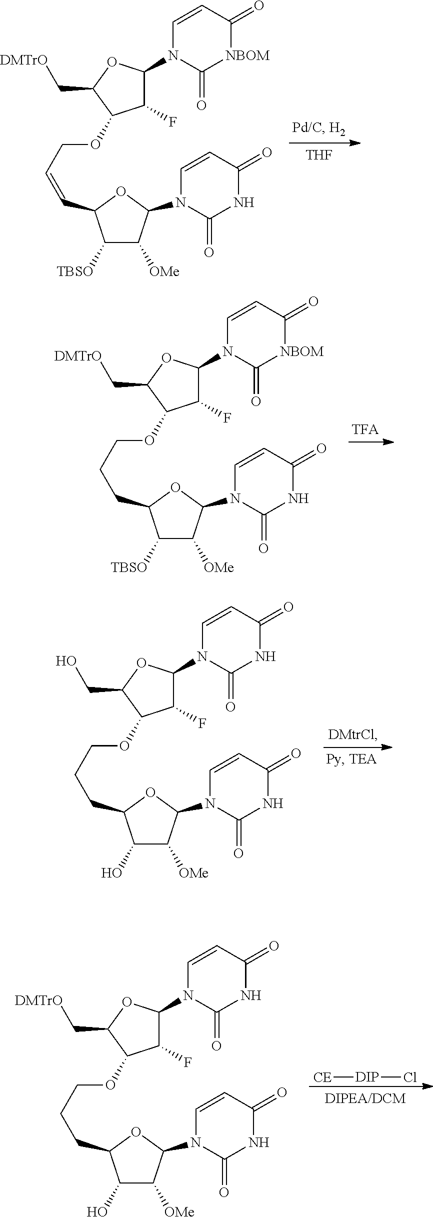

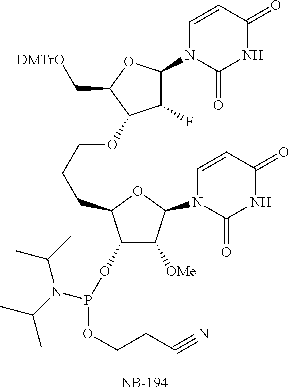

### Step 1: 3-((benzyloxy)methyl)-1-((2R,3R,4R,5R)-5-((bis(4-methoxyphenyl)(phenyl) methoxy) methyl)-4-(3-((2R,3R,4R,5R)-3-((tert-butyldimethylsilyl)oxy)-5-(2,4-dioxo-3,4-dihydropyrimidin-1(2H)-yl)-4-methoxytetrahydrofuran-2-yl)propoxy)-3-fluorotetrahydrofuran-2-yl)pyrimidine-2,4(1H,3H)-dione

To a stirred solution of 3-((benzyloxy)methyl)-1-((2R,3R,4R,5R)-5-((bis(4-methoxyphenyl) (phenyl) methoxy)methyl)-4-(((Z)-3-((2R,3R,4R,5R)-3-((tert-butyldimethylsilyl)oxy)-5-(2,4-dioxo-3,4dihydro pyrimidin-1(2H)-yl)-4-methoxytetrahydrofuran-2-yl)allyl)oxy)-3-fluorotetrahydro-furan-2-yl)pyrimidine-2,4(1H,3H)-dione (2.2 g, 2.1 mmol) in THF (40 mL) were added Pd/C (0.44 g, 4.14 mmol, 20%). The resulting mixture was stirred overnight at 25° C. under H₂ (balloon pressure) atmosphere. The reaction mixture was filtered. The filter cake was washed with MeOH (200 mL) and then CH₂Cl₂ (200 mL). After filtration, the filtrate was concentrated under reduced pressure to obtain the titled compound (2.0 g, 90% yield) as a black solid which was used in the next step without purification. MS (ESI): m/z=1073.0 [M+Na]⁺.

### Step 2: 1-((2R,3R,4R,5R)-4-(3-((2R,3R,4R,5R)-5-(2,4-dioxo-3,4-dihydropyrimidin-1(2H)-yl)-3-hydroxy-4-methoxytetrahydrofuran-2-yl)propoxy)-3-fluoro-5-(hydroxymethyl)tetrahydrofuran-2-yl)pyrimidine-2,4(1H,3H)-dione

Added TFA (10 mL) in portions to 3-((benzyloxy)methyl)-1-((2R,3R,4R,5R)-5-((bis(4-methoxy phenyl)(phenyl)methoxy)methyl)-4-(3-((2R,3R,4R,5R)-3-((tert-butyldimethylsilyl)oxy)-5-(2,4-dioxo-3,4-dihydropyrimidin-1(2H)-yl)-4-methoxytetrahydrofuran-2-yl)propoxy)-3-fluorotetrahydrofuran-2-yl) pyrimidine-2,4(1H,3H)-dione (2.0 g, 1.9 mmol) at room temperature at N2 atmosphere. The resulting mixture was stirred for 30 min at 80° C. at N2 atmosphere. The residue was quenched with the addition of saturated NaHCO₃ solution at room temperature until pH>7. The aqueous layers was purified by reversed-phase flash chromatography with the following conditions: column, C18 silica gel; mobile phase, ACN in water, 10 to 90% gradient in 20 min; detector, UV 254/220 nm to afford the titled compound (0.66 g, 67% yield) as a light yellow solid. MS (ESI): m/z=537.3 [M+Na]⁺.

### Step 3: 1-((2R,3R,4R,5R)-5-((bis(4-methoxyphenyl)(phenyl)methoxy)methyl)-4-(3-((2R,3R,4R,5R)-5-(2,4-dioxo-3,4-dihydropyrimidin-1(2H)-yl)-3-hydroxy-4-methoxytetrahydrofuran-2-yl)propoxy)-3-fluorotetrahydrofuran-2-yl)pyrimidine-2,4(1H,3H)-dione

To a stirred solution of 1-((2R,3R,4R,5R)-4-(3-((2R,3R,4R,5R)-5-(2,4-dioxo-3,4-dihydropyrimidin-1(2H)-yl)-3-hydroxy-4-methoxytetrahydrofuran-2-yl)propoxy)-3-fluoro-5-(hydroxymethyl)tetrahydrofuran-2-yl)pyrimidine-2,4(1H,3H)-dione (1.04 g, 2.022 mmol) in pyridine (10 mL) were added TEA (0.6 mL, 4.0 mmol) and 1-[chloro(4-methoxyphenyl)phenylmethyl]-4-methoxybenzene (1.03 g, 3.033 mmol) in portions at 0° C. at N2 atmosphere. The resulting mixture was stirred for 3 hours at RT under N2 atmosphere. The reaction was quenched with several drops of methanol. The resulting solution was concentrated under reduced pressure. The reaction was further quenched by the addition of water (10 mL) at room temperature. The resulting mixture was extracted with EA (3×200 mL). The combined organic layers were washed with brine, dried by Na₂SO₄. After filtration, the filtrate was concentrated under reduced pressure. The residue was purified by C18 column, eluted with CH₃CN/H₂O to afford the titled compound (1.04 g, 1.27 mmol, 63% yield) as a yellow solid. MS (ESI, negative mode): m/z=815.4 [M−H]⁻.

### Step 4: (2R,3R,4R,5R)-2-(3-(((2R,3R,4R,5R)-2-((bis(4-methoxyphenyl)(phenyl) methoxy)methyl)-5-(2,4-dioxo-3,4-dihydropyrimidin-1(2H)-yl)-4-fluorotetrahydrofuran-3-yl)oxy)propyl)-5-(2,4-dioxo-3,4-dihydropyrimidin-1(2H)-yl)-4-methoxytetrahydrofuran-3-yl (2-cyanoethyl) diisopropylphosphoramidite

Added N,N-diisopropyl chlorophosphoramidite (0.9 mL, 3.7 mmol) to a solution of 1-((2R,3R,4R,5R)-5-((bis(4-methoxyphenyl)(phenyl)methoxy)methyl)-4-(3-((2R,3R,4R,5R)-5-(2,4-dioxo-3,4-dihydropyrimidin-1(2H)-yl)-3-hydroxy-4-methoxytetrahydrofuran-2-yl)propoxy)-3-fluorotetrahydrofuran-2-yl)pyrimidine-2,4(1H,3H)-dione (1.0 g, 1.22 mol) and DIPEA (2 mL, 11 mmol) in DCM (12 mL) at room temperature. The reaction was allowed to stir for 2 hours. The reaction was quenched by pouring the reaction into saturated aqueous NaHCO₃ (20 mL) and extracted with DCM (3×300 mL). The organic layer was separated, dried with Na₂SO₄, filtered and concentrated. The crude was purified by flash column on 40 g silica gel column (column with pretreated with 1% Et₃N with hexane) with 0 to 100% EA/H to afford the titled compound NB-194 (0.70 g, 56% yield) as a white solid. ³¹P NMR (202 MHz, CDCl₃) δ 150.44, 150.2 ppm. MS (ESI): m/z=1038.9 [M+Na]⁺.

Oligonucleotide Comprising Dinucleotide NB-194 has been Synthesized Using the General Procedure Described in Example 1.

### Example 84: NB-195

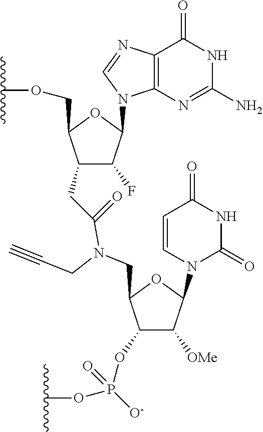 [SMILES: *OC[C@H]1O[C@@H](n2cnc3c(=O)[nH]c(N)nc32)[C@H](F)[C@@H]1CC(=O)N(CC#C)C[C@H]1O[C@@H](n2ccc(=O)[nH]c2=O)[C@H](OC)[C@@H]1OP(=O)([O-])O*]

### Synthesis of NB-195

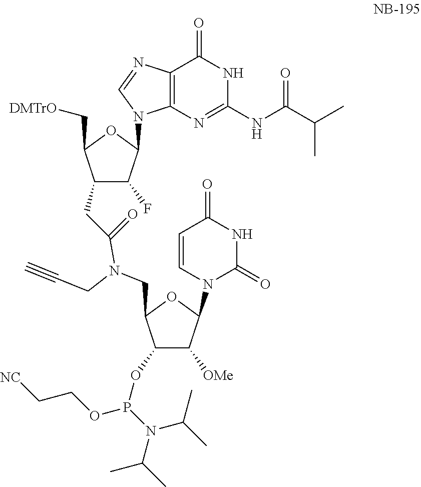

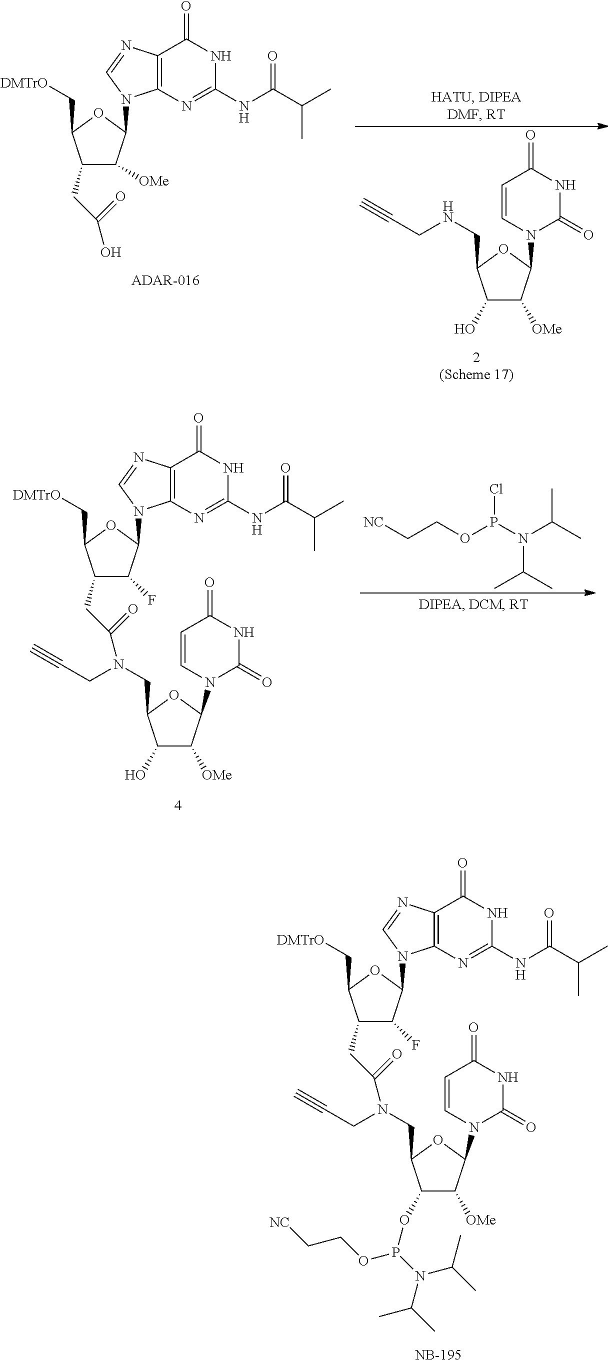

A solution of acid ADAR-016 (5 g, 7.153 mmol, 1 eq), DIPEA (3.7 mL, 21.45 mmol, 3 eq), HATU (4.07 g, 10.73 mmol, 1.5 eq) and amine 2 (2.32 g, 7.86 mmol, 1.1 eq) in DMF (50 mL) was stirred for 3 h at room temperature, reaction mixture was added dropwise to a vigorously stirring water (250 ml), precipitated solids were filtered and washed with water, the solids were dried and re dissolved in DCM dried over Na₂SO₄ and concentrated, the residue was dissolved in EtOAc then purified by column chromatography using Ethyl acetate/MeOH, 0-20% as an eluent, pure fractions were combined and concentrated to obtain amide 1 (4.5 g, 62%) as an off white solid. LCMS m/z 978 (M+1).

To a stirred solution of alcohol 4 (2.8 g, 2.866 mmol, 1 eq) and diisopropylethylamine (3 mL, 17.2 mmol, 6 eq), in DCM 30 mL), was added N, N-diisopropyl chlorophosphoramidite (1.9 mL 8.5 mmol, 3 eq) dropwise. The reaction mixture was stirred at room temperature for 3 h. Reaction mixture was quenched with Aq. Saturated NaHCO₃ solution (50 mL), extracted with DCM (200) ml, washed with brine (100 mL), dried over Na₂SO₄, and evaporated the crude product was dissolved in DCM, loaded on to (pre-equilibrated with 1% Et₃N-Hexane) Biotage silica gel column (100 g 20 μm), and purified by flash chromatography using Hexane/EtOAc/MeOH 0-0% 1 CV-Hex, 0-100% 3 CV-Hex/EA, then 0-10% 10 CV-EA-MeOH containing 1% Et₃N as an additive. Pure fractions were combined and concentrated, dried under high vacuum to obtain Phosphoramidite NB-195, (1.71 g, 50%) as a white solid. 92% purity by HPLC, 100% pure by P31-NMR, Mass (m/z 1177 M+), ³¹P NMR (202, MHz, DMSO-d6) δ 149.97, 149.73, 149.53, 149.49.

### Oligonucleotide Comprising Dinucleotide NB-195 to be Synthesized Using the General Procedure Described in Example 1.

### Example 85: NB-196

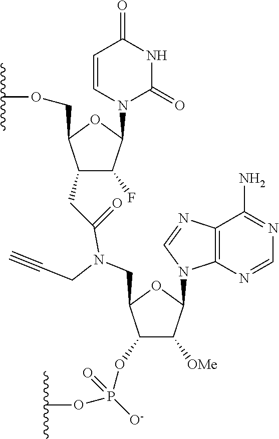 [SMILES: *OC[C@H]1O[C@@H](n2ccc(=O)[nH]c2=O)[C@H](F)[C@@H]1CC(=O)N(CC#C)C[C@H]1O[C@@H](n2cnc3c(N)ncnc32)[C@H](OC)[C@@H]1OP(=O)([O-])O*]

### Synthesis of NB-196

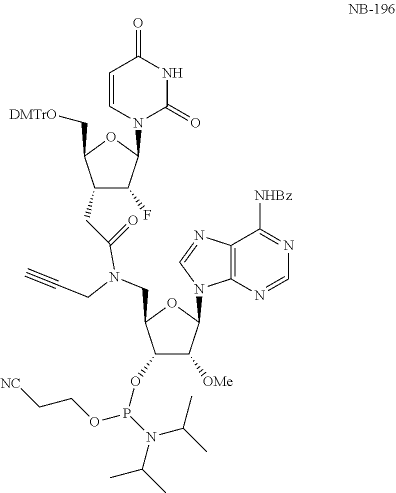

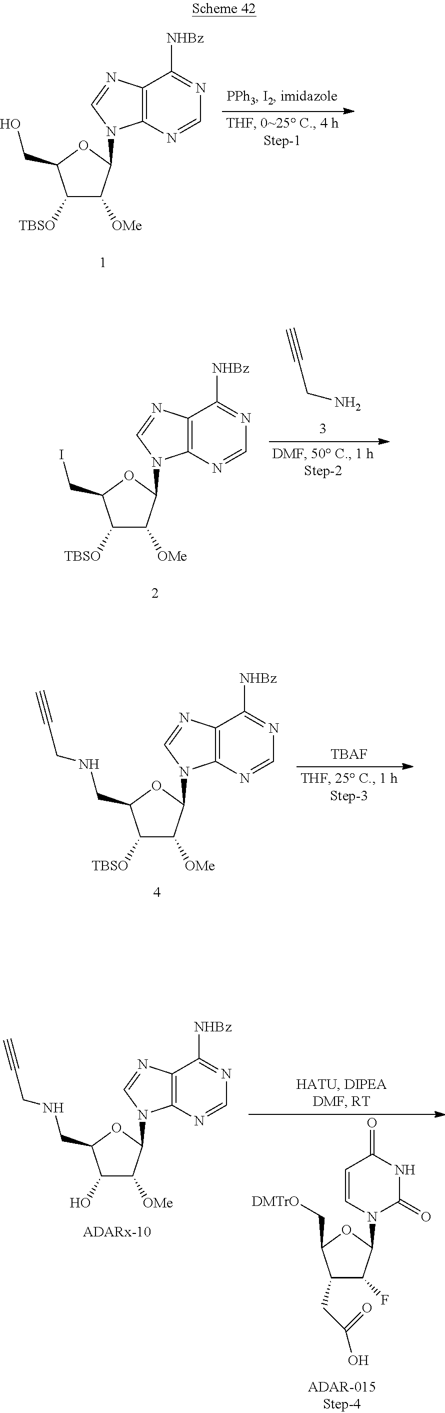

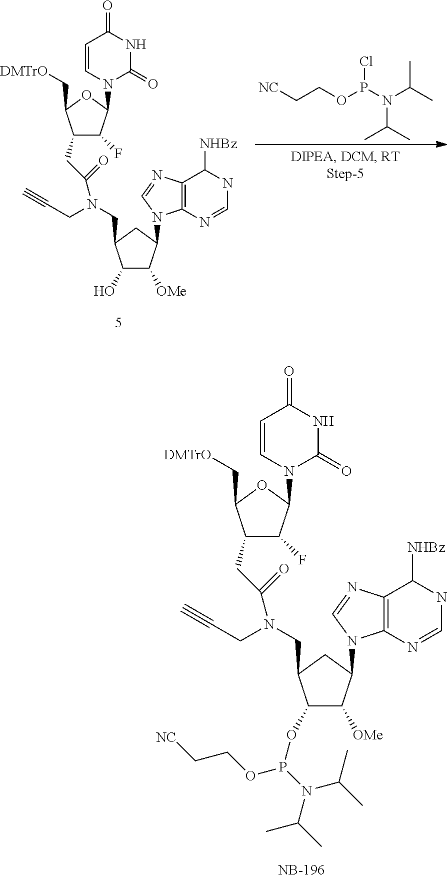

Step 1: To a solution of I₂ (3.05 g, 12.01 mmol, 2.42 mL, 2 eq) in THF (15 mL) was added dropwise to a mixture of N-[9-[(2R,3R,4R,5R)-4-[tert-butyl(dimethyl)silyl]oxy-5-(hydroxymethyl)-3-methoxy-tetrahydrofuran-2-yl]purin-6-yl]benzamide 1 (3 g, 6.00 mmol, 1 eq), PPh₃ (3.15 g, 12.01 mmol, 2 eq) and imidazole (1.23 g, 18.01 mmol, 3 eq) in THF (30 mL) at 0° C. The mixture was degassed and purged with N2 for 3 times, and then was stirred at 25° C. for 4 hr under N2 atmosphere. LCMS showed the desired compound was formed.

The reaction mixture was concentrated under reduced pressure to give a residue. The residue was purified by flash silica gel chromatography (ISCO®; 20 g SepaFlash® Silica Flash Column, Eluent of 0-35% Ethyl acetate/Petroleum ethergradient @60 mL/min) to give the Iodo compound 2 (4.7 g, crude) as a yellow solid. MS ES⁺: 610.4

Step 2: To a solution of N-[9-[(2R,3R,4S,5S)-4-[tert-butyl(dimethyl)silyl]oxy-5-(iodomethyl)-3-methoxy-tetrahydrofuran-2-yl]purin-6-yl]benzamide 2 (3.3 g, 5.41 mmol, 1 eq), prop-2-yn-1-amine 3 (1.49 g, 27.07 mmol, 1.73 mL, 5 eq) was added DMF (30 mL). The mixture was stirred at 50° C. for 1 hr. LCMS showed the desired compound was formed. The reaction mixture was concentrated under reduced pressure to give a residue. The residue was purified by flash silica gel chromatography (ISCO®; 120 g SepaFlash® Silica Flash Column, Eluent of 0˜85% Ethyl acetate/Petroleum ethergradient @45 mL/min) to give the Alkyne 3 (220 mg, 0.409 mmol, 7.57% yield) as a yellow solid. MS ES⁺: 537.5, ¹H NMR (400 MHz, DMSO-d₆) δ=11.26 (br s, 1H), 8.78 (br d, J=7.4 Hz, 2H), 8.05 (br d, J=7.4 Hz, 2H), 7.69-7.61 (m, 1H), 7.56 (br t, J=7.5 Hz, 2H), 6.21-6.14 (m, 1H), 4.81-4.63 (m, 2H), 3.85-3.55 (m, 2H), 3.32 (s, 3H), 3.30 (br s, 1H), 3.27-3.08 (m, 2H), 0.94 (s, 9H), 0.16 (d, J=4.9 Hz, 6H).

Step 3: To a solution of N-[9-[(2R,3R,4R,5R)-4-[tert-butyl(dimethyl)silyl]oxy-3-methoxy-5-[(prop-2-ynylamino)methyl]tetrahydrofuran-2-yl]purin-6-yl]benzamide 4 (1.4 g, 2.61 mmol, 1 eq), TBAF (1 M, 3.39 mL, 1.3 eq) in THF (15 mL). The mixture was stirred at 25° C. for 1 hr. LCMS showed the desired compound was formed. The reaction mixture was concentrated under reduced pressure to give a residue. The residue was purified by flash silica gel chromatography (ISCO®; 20 g SepaFlash® Silica Flash Column, Eluent of 0-3% MeOH/DCM @40 mL/min) to give ADARx-10 (0.9 g, 2.00 mmol, 76.77% yield, 94% purity) as a yellow solid. MS ES⁺: 423.3 ¹H NMR (400 MHz, DMSO-d₆) δ=11.23 (br s, 1H), 8.75 (d, J=13.3 Hz, 2H), 8.05 (d, J=7.5 Hz, 2H), 7.70-7.61 (m, 1H), 7.60-7.51 (m, 2H), 6.11 (d, J=6.1 Hz, 1H), 5.34 (d, J=5.4 Hz, 1H), 4.53 (t, J=5.6 Hz, 1H), 4.41-4.33 (m, 1H), 4.11-3.98 (m, 1H), 3.39-3.36 (m, 3H), 3.31 (br s, 2H), 3.07 (t, J=2.2 Hz, 1H), 2.94-2.78 (m, 2H).

Step 3: A solution of acid ADAR-015 (1.2 g, 2.034 mmol, 1 eq), DIPEA (1 mL, 6.1 mmol, 3 eq), HATU (1.16 g, 3.05 mmol, 1.5 eq) and amine ADARx-10 (0.9 g, 2.13 mmol, 1.05 eq) in DMF (10 mL) was stirred for 3 h at room temperature, reaction mixture was added dropwise to a vigorously stirring brine solution (100 ml), precipitated solids were filtered and washed with water, the solids were dried and re dissolved in DCM dried over Na₂SO₄ and concentrated, the residue was dissolved in EtOAc then purified by column chromatography using Ethyl acetate/MeOH, 0-20% as an eluent, pure fractions were combined and concentrated to obtain amide 5 (1.1 g, 52%) as an off white solid. LCMS m/z 996 (M+1).

Step 4: To a stirred solution of alcohol 5 (2.8 g, 2.866 mmol, 1 eq) and diisopropylethylamine (3 mL, 17.2 mmol, 6 eq), in DCM 30 mL), was added N, N-diisopropyl chlorophosphoramidite (1.9 mL 8.5 mmol, 3 eq) dropwise. The reaction mixture was stirred at room temperature for 3 h. Reaction mixture was quenched with Aq. Saturated NaHCO₃ solution (50 mL), extracted with DCM (200) ml, washed with brine (100 mL), dried over Na₂SO₄, and evaporated the crude product was dissolved in DCM, loaded on to (pre-equilibrated with 1% Et₃N-Hexane) Biotage silica gel column (100 g 20 μm), and purified by flash chromatography using Hexane/EtOAc/MeOH 0-0% 1 CV-Hex, 0-100% 3 CV-Hex/EA, then 0-10% 10 CV-EA-MeOH containing 1% Et₃N as an additive. Pure fractions were combined and concentrated, dried under high vacuum to obtain Phosphoramidite NB-196, 1.11 g, 91% as aa off white solid. 95% purity by HPLC, 90% purity by P31-NMR, Mass (m/z 1195 M+), ³¹P NMR (202, MHz, DMSO-d6) δ 15.028, 150.01 149.72, 149.63.

### Oligonucleotide Comprising Dinucleotide NB-196 to be Synthesized Using the General Procedure Described in Example 1.

### Example 86: NB-197

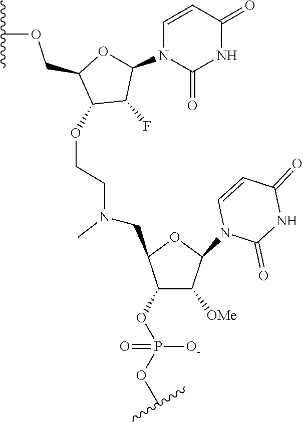

### Synthesis of NB-197: (2R,3R,4R,5R)-2-(((2-(((2R,3R,4R,5R)-2-((bis(4-methoxyphenyl)(phenyl)methoxy) methyl)-5-(2,4-dioxo-3,4-dihydropyrimidin-1(2H)-yl)-4-fluorotetrahydrofuran-3-yl)oxy)ethyl)(methyl)-amino)methyl)-5-(2,4-dioxo-3,4-dihydropyrimidin-1(2H)-yl)-4-methoxytetrahydrofuran-3-yl(2-cyanoethyl) diisopropylphosphoramidite

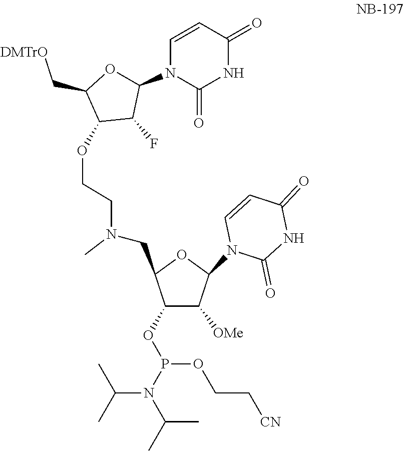

The synthesis of NB-197 followed the same procedure as in NB-186 by replacing 2′-O-methyluridine with 2′-fluoro-2′-deoxyuridine in step 4 to obtain the titled compound NB-197 as a white solid. ³¹P NMR (202 MHz, CDCl₃) δ 150.28, 150.13. MS (ESI): 1046.7 [M+H]⁺, 1068.6 [M+Na]⁺.

Oligonucleotide Comprising Dinucleotide NB-197 has been Synthesized Using the General procedure described in example 1.

### Example 87: NB-198

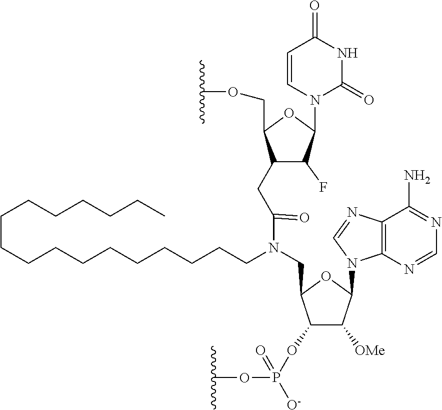

### Synthesis of NB-198

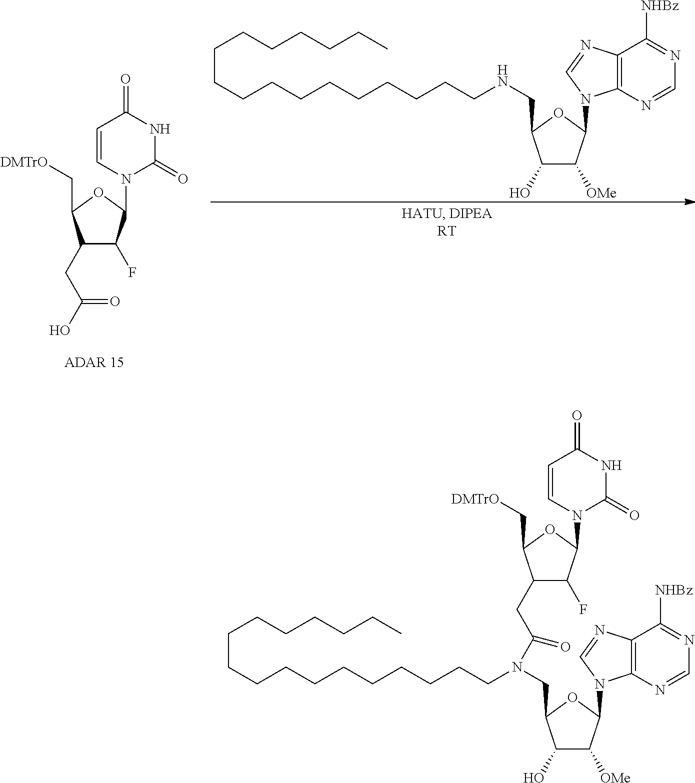

N-(9-((2S,3R,4R,5R)-5-((2-((2R,3S,4S,5S)-2-((bis(4-methoxyphenyl)(phenyl) methoxy)methyl)-5-(2,4-dioxo-3,4-dihydropyrimidin-1(2H)-yl)-4-fluorotetrahydrofuran-3-yl)-N-heptadecylacetamido)methyl)-4-hydroxy-3-methoxytetrahydrofuran-2-yl)-9H-purin-6-yl)benzamide. A solution of acid (8 g, 13.6 mmol, 1 eq), amine (6.5 g, 14.9 mmol, 1.3 eq) and HATU (7.7 g, 20.3 mmol, 1.5 eq) in DMF (40 mL) was added DIPEA (8 mL, 40.7 mmol, 3 eq) at RT and the mixture was stirred for 1 hr 30 min at room temperature. Reaction mixture was added to a stirring solution of sat. NaHCO₃, precipitated solids were filtered and washed with water and dried under high vac overnight. Purified by flash chromatography (100 g 20 micron Biotage column) using MeOH/EtOAc, 0-20% 5 CV then 20% 10 CV as an eluent, to obtain amide (7.36 g, 48.4%) as a beige solid.

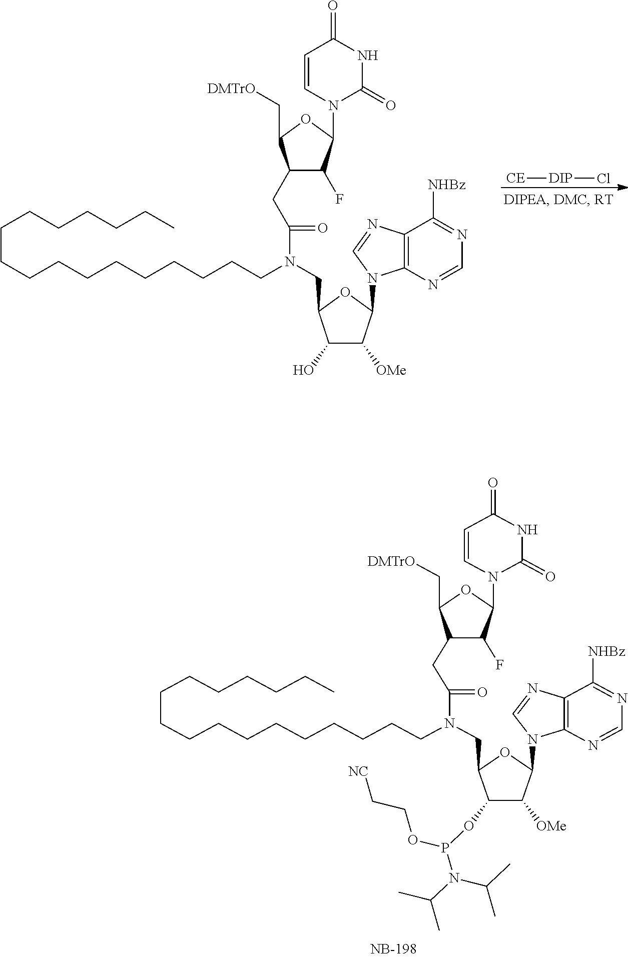

(2R,3R,4R,5S)-5-(6-benzamido-9H-purin-9-yl)-2-((2-((2S,3R,4R,5R)-2-((bis(4-methoxyphenyl)(phenyl)methoxy)methyl)-5-(2,4-dioxo-3,4-dihydropyrimidin-1(2H)-yl)-4-fluorotetrahydrofuran-3-yl)-N-heptadecylacetamido)methyl)-4-methoxytetrahydrofuran-3-yl (2-cyanoethyl) diisopropylphosphoramidite. To a stirred solution of alcohol (7.36 g, 6.16 mmol, 1 eq) and diisopropylethylamine (6.4 mL, 37.0 mmol, 6 eq), in DCM (45 mL), was added N, N-diisopropyl chlorophosphoramidite (4.3 mL 18.5 mmol, 3 eq) dropwise. The reaction mixture was stirred at room temperature for 3 h. reaction mixture was quenched diluted with Aq. Saturated NaHCO₃ solution (100 mL), extracted with DCM (100) ml, washed with brine (100 mL), dried over Na₂SO₄, and evaporated the crude product was loaded on to (pre-equilibrated with 1% Et₃N-EtOAc) Biotage silica gel column (100 g 20 μm), and purified by flash chromatography using MeOH/EtOAc 0-1% 10 CV, 1-50% 2 CV, 50% 2 CV containing 1% Et₃N as an additive, gave 6.32 g (73%) of NB-198. ¹H NMR (500 MHz, d-DMSO): δ 11.5-11.0 (br, 3H), 9.0-8.0 (m, 4H), 7.8-7.6 (m, 3H), 7.4-7.1 (m, 9H), 7.4-7.2 (m, 10H), 6.8 (m, 4H), 6.3 (m, 1H), 5.8-5.4 (m, 3H), 4.5 (m, 1H), 4.3-4.0 (m, 5H), 3.9-3.5 (m, 15H), 3.5-3.1 (m, 10H), 2.8-2.5 (m, 14H), 2.6-2.5 (m, 2H), 1.2-1.1 (m, 20H). ³¹P NMR (202, MHz, DMSO-d6) δ 150.19, 149.86, 149.72, 149.57. Mass (ESI) m/z: [M+Na]⁺ Cacld for 1418.27, found: 1418.32

Oligonucleotide Comprising Dinucleotide NB-198 has been Synthesized Using the General Procedure Described in Example 1.

### Example 88: NB-199

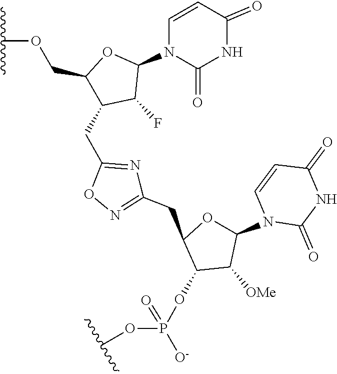 [SMILES: *OC[C@H]1O[C@@H](n2ccc(=O)[nH]c2=O)[C@H](F)[C@@H]1Cc1nc(C[C@H]2O[C@@H](n3ccc(=O)[nH]c3=O)[C@H](OC)[C@@H]2OP(=O)([O-])O*)no1]

### Synthesis of NB-199: (2R,3R,4R,5R)-2-((5-(((2S,3R,4R,5R)-2-((bis(4-methoxyphenyl)(phenyl)methoxy)methyl)-5-(2,4-dioxo-3,4-dihydropyrimidin-1(2H)-yl)-4-fluorotetrahydrofuran-3-yl)methyl)-1,2,4-oxadiazol-3-yl)methyl)-5-(2,4-dioxo-3,4-dihydropyrimidin-1(2H)-yl)-4-methoxytetrahydrofuran-3-yl (2-cyanoethyl) diisopropylphosphoramidite

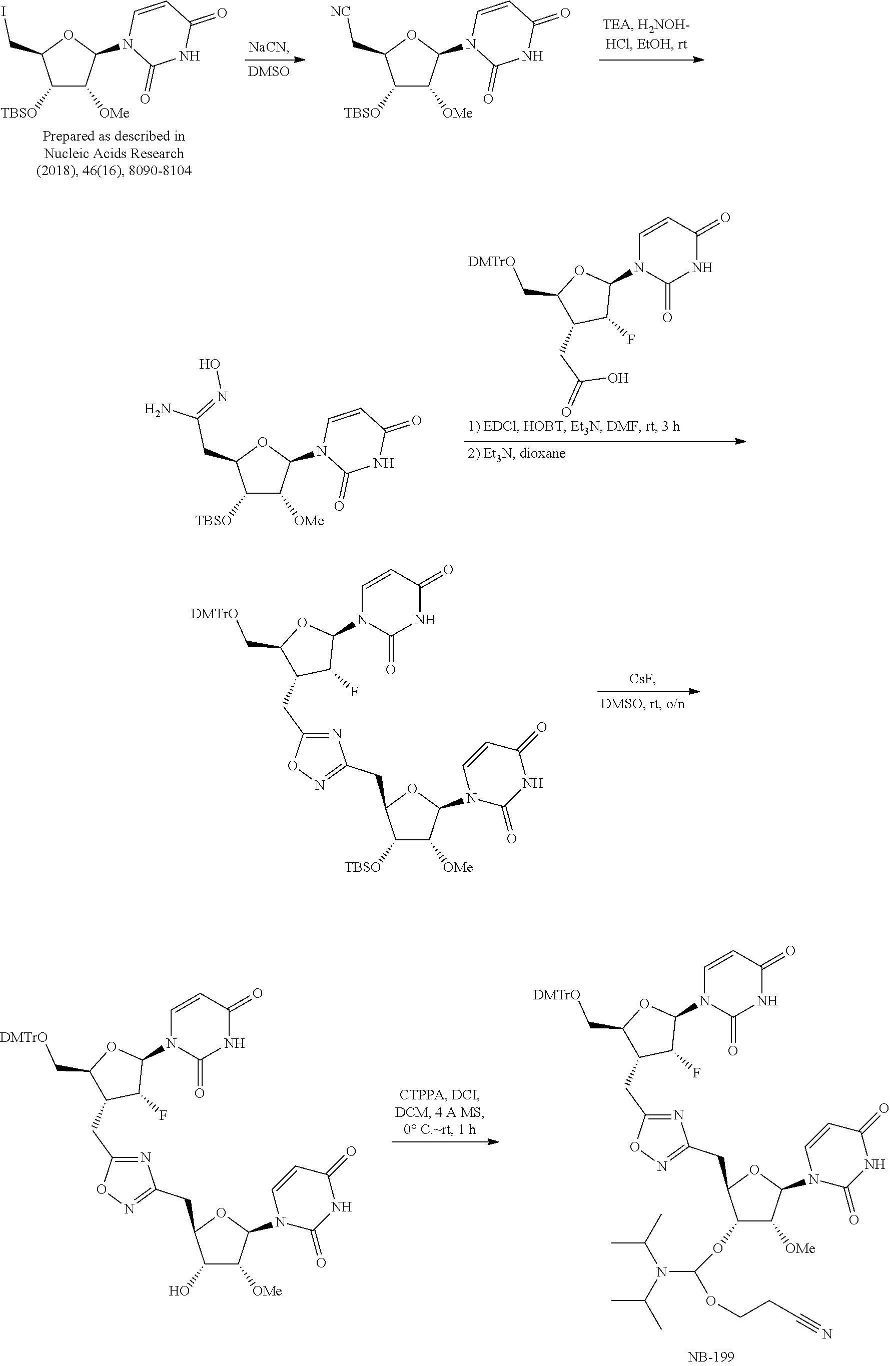

### Step 1: 2-((2R,3R,4R,5R)-3-((tert-butyldimethylsilyl)oxy)-5-(2,4-dioxo-3,4-dihydropyrimidin-1(2H)-yl)-4-methoxytetrahydrofuran-2-yl)acetonitrile

To a solution of 1-((2R,3R,4S,5S)-4-((tert-butyldimethylsilyl)oxy)-5-(iodomethyl)-3-methoxy tetrahydrofuran-2-yl)pyrimidine-2,4(1H,3H)-dione (45 g, 93 mmol) in DMSO (450 mL) followed by addition of NaCN (9.14 g, 187 mmol). The mixture was stirred at RT for 4 hours. The reaction was quenched by addition of saturated aqueous FeSO₄ (100 mL). The aqueous layer was extracted with ethyl acetate (3×400 mL). Combined the ethyl acetate layers, washed with water (2×100 mL) and brine (100 mL). The combined organic layers were dried over anhydrous Na₂SO₄. After filtration, the filtrate was concentrated under reduced pressure. The residue was subjected to silica column purification and eluted with (EA:PE=4:1) to afford the titled compound (7.9 g, 41% yield) as a white solid. MS(ESI) m/z=382.2[M+H]⁺.

### Step 2: 2-((2R,3R,4R,5R)-3-((tert-butyldimethylsilyl)oxy)-5-(2,4-dioxo-3,4-dihydropyrimidin-1(2H)-yl)-4-methoxytetrahydrofuran-2-yl)-N′-hydroxyacetimidamide

To a solution of 2-((2R,3R,4R,5R)-3-((tert-butyldimethylsilyl)oxy)-5-(2,4-dioxo-3,4-dihydro-pyrimidin-1(2H)-yl)-4-methoxytetrahydrofuran-2-yl)acetonitrile (2.0 g, 5.2 mmol) in EtOH (20 mL), followed by the addition of TEA (1.0 g, 9.9 mmol), hydroxylamine hydrochloride (0.54 g, 7.8 mmol). The reaction mixture was stirred at 50° C. under argon atmosphere for 2 days. EtOH was removed under reduced pressure to afford a solid and DCM (10 mL) was added to get more solid out of the reaction. The mixture was filtered, and the filter cake was washed with tert-butyl methyl ether (50 mL). The filtrate was concentrated to provide the title compound (1.5 g, 66% yield) as a white solid. MS(ESI) m/z=415.2 [M+H]⁺.

### Step 3: 1-((2R,3R,4R,5S)-5-((bis(4-methoxyphenyl)(phenyl)methoxy)methyl)-4-((3-(((2R,3R,4R,5R)-3-((tert-butyldimethylsilyl)oxy)-5-(2,4-dioxo-3,4-dihydropyrimidin-1(2H)-yl)-4-methoxytetrahydrofuran-2-yl)methyl)-1,2,4-oxadiazol-5-yl)methyl)-3-fluorotetrahydrofuran-2-yl)pyrimidine-2,4(1H,3H)-dione

To a stirred solution of 2-((2S,3R,4R,5R)-2-((bis(4-methoxyphenyl)(phenyl) methoxy)methyl)-5-(2,4-dioxo-3,4-dihydropyrimidin-1(2H)-yl)-4-fluorotetrahydrofuran-3-yl)acetic acid (6.42 g, 10.9 mmol) in DMF (90 mL) were added EDCI (4.17 g, 21.7 mmol), HOBT (2.93 g, 21.7 mmol) at room temperature. The resulting mixture was stirred for 30 minutes at room temperature under nitrogen atmosphere. To the above mixture was added TEA (2.20 g, 21.7 mmol) and 2-((2R,3R,4R,5R)-3-((tert-butyldimethylsilyl)oxy)-5-(2,4-dioxo-3,4-dihydropyrimidin-1(2H)-yl)-4-methoxytetrahydrofuran-2-yl)-N′-hydroxyacetimidamide (4.5 g, 11 mmol) at room temperature. The resulting mixture was stirred for 3 hours at room temperature. The reaction was extracted with EA (3×100 mL). The combined organic layers were washed with brine (50 mL) and dried over sodium sulfate. The mixture was filtered and concentrated. The residue was re-dissolved in dioxane (90 mL) and TEA (2.20 g, 21.7 mmol). The resulting mixture was stirred for 8 hours at 100° C. under nitrogen atmosphere. The reaction was cooled and extracted with EA (2×500 mL). The combined organic layers were washed brine (100 mL) and dried over sodium sulfate. The mixture was filtered and concentrated. The residue was purified by column C18 (ACN/H₂O, from 10 to 100%) to afford the titled compound (5.0 g, 43% yield) as a yellow solid, MS (ESI, negative mode): m/z=967.5 [M−H]⁻.

### Step 4: 1-((2R,3R,4R,5S)-5-((bis(4-methoxyphenyl)(phenyl)methoxy)methyl)-4-((3-(((2R,3R,4R,5R)-5-(2,4-dioxo-3,4-dihydropyrimidin-1(2H)-yl)-3-hydroxy-4-methoxytetrahydrofuran-2-yl)methyl)-1,2,4-oxadiazol-5-yl)methyl)-3-fluorotetrahydrofuran-2-yl)pyrimidine-2,4(1H,3H)-dione

To a stirred solution of 1-((2R,3R,4R,5S)-5-((bis(4-methoxyphenyl)(phenyl) methoxy)methyl)-4-((3-(((2R,3R,4R,5R)-3-((tert-butyldimethylsilyl)oxy)-5-(2,4-dioxo-3,4-dihydropyrimidin-1(2H)-yl)-4-methoxy tetrahydrofuran-2-yl)methyl)-1,2,4-oxadiazol-5-yl)methyl)-3-fluorotetrahydrofuran-2-yl)pyrimidine-2,4(1H,3H)-dione (4.5 g, 4.6 mmol) in DMSO (45 mL) were added CsF (1.41 g, 9.3 mmol) at room temperature. The resulting mixture was stirred overnight at room temperature under nitrogen atmosphere. The reaction mixture was quenched with H₂O (100 mL) and extracted with EA (3×100 mL). The combined organic layers were washed with brine (100 mL) and dried by anhydrous Na₂SO₄.

After filtration, the filtrate was concentrated under reduced pressure. The residue was purified by C18 (ACN/H₂O) to afford the titled compound (3.6 g, 81% yield) as a white solid. MS(ESI, negative mode): m/z=853.4 [M−H]⁻.

### Step 5: (2R,3R,4R,5R)-2-((5-(((2S,3R,4R,5R)-2-((bis(4-methoxyphenyl)(phenyl) methoxy)methyl)-5-(2,4-dioxo-3,4-dihydropyrimidin-1(2H)-yl)-4-fluorotetrahydrofuran-3-yl)methyl)-1,2,4-oxa-diazol-3-yl)methyl)-5-(2,4-dioxo-3,4-dihydropyrimidin-1(2H)-yl)-4-methoxytetrahydrofuran-3-yl (2-cyanoethyl) diisopropylphosphoramidite

To a solution of 2,4-dicyanoimidazole (0.64 g, 5.44 mmol), 4 A MS in DCM (15 mL) were added 2-cyanoethyl N,N,N′,N′-tetraisopropylphosphorodiamidite (1.64 g, 5.44 mmol), the reaction was stirred at room temperature for 10 min under argon atmosphere. 1-((2R,3R,4R,5S)-5-((bis(4-methoxyphenyl) (phenyl)methoxy)methyl)-4-((3-(((2R,3R,4R,5R)-5-(2,4-dioxo-3,4-dihydro-pyrimidin-1(2H)-yl)-3-hydroxy-4-methoxytetrahydrofuran-2-yl)methyl)-1,2,4-oxadiazol-5-yl)methyl)-3-fluorotetrahydrofuran-2-yl) pyrimidine-2,4(1H,3H)-dione (3.1 g, 3.63 mmol) in DCM (16 mL) was added dropwise at RT into the reaction mixture and the reaction was stirred at room temperature for 1 hour under argon atmosphere. The reaction was diluted with DCM (500 mL, contains 0.5% TEA) and filtered.

The organic layer was washed with NaHCO₃ (sat, 200 mL) and dried over magnesium sulfate. The mixture was filtered and concentrated. The residue was purified with silica gel (1% TEA in DCM/MeOH) for 2 times and followed by combi-Flash with the following conditions: Column, C18 gel (330 g); mobile phase, water (5 mmol/L NH₄HCO₃) and acetonitrile (50% to 100% acetonitrile in 15 min, hold 100% 5 mins to afford the titled compound NB-199 (2.48 g, 64.9% yield) as a white solid. ³¹P NMR (202 MHz, DMSO-d₆) δ 150.28, 149.78. MS(ESI): m/z=1077.3 [M+Na]⁺.

### Oligonucleotide Comprising Dinucleotide NB-199 to be Synthesized Using the General Procedure Described in Example 1.

### Example 89: NB-200

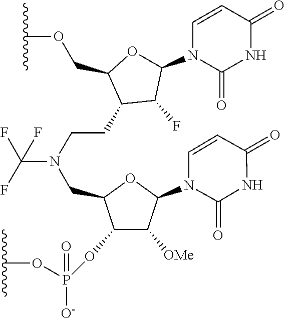 [SMILES: *OC[C@H]1O[C@@H](n2ccc(=O)[nH]c2=O)[C@H](F)[C@@H]1CCN(C[C@H]1O[C@@H](n2ccc(=O)[nH]c2=O)[C@H](OC)[C@@H]1OP(=O)([O-])O*)C(F)(F)F]

### Synthesis of NB-200: (2R,3R,4R,5R)-2-(((2-((2S,3R,4R,5R)-2-((bis(4-methoxyphenyl)(phenyl)methoxy)methyl)-5-(2,4-dioxo-3,4-dihydropyrimidin-1(2H)-yl)-4-fluorotetrahydrofuran-3-yl)ethyl)(trifluoromethyl) amino)methyl)-5-(2,4-dioxo-3,4-dihydropyrimidin-1(2H)-yl)-4-methoxytetrahydrofuran-3-yl (2-cyanoethyl) diisopropylphosphoramidite

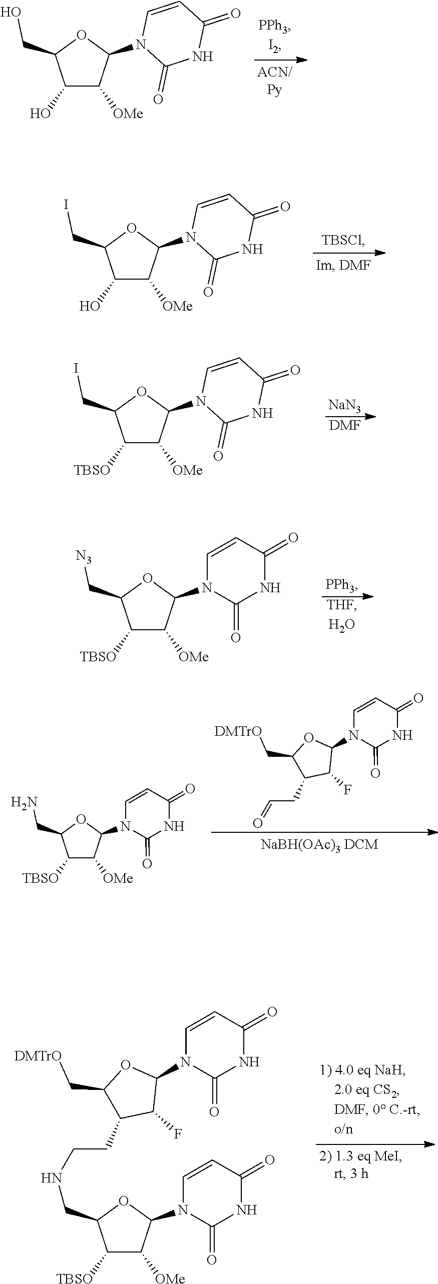

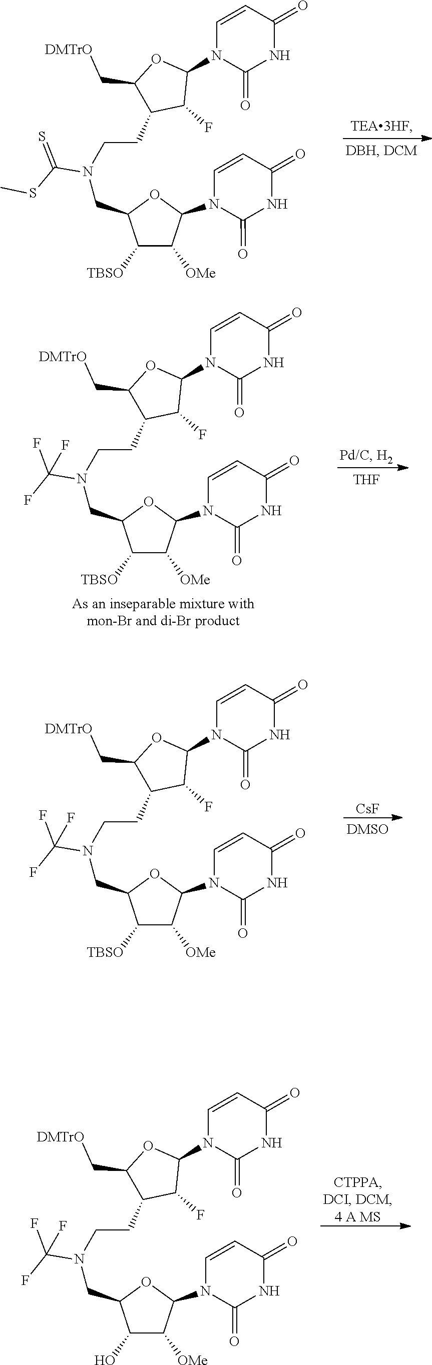

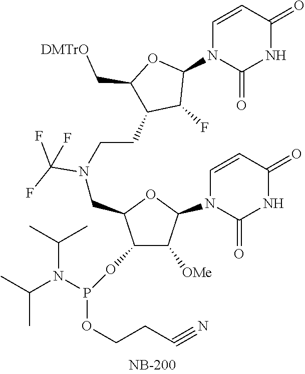

### Step 1: 1-((2R,3R,4S,5S)-4-hydroxy-5-(iodomethyl)-3-methoxytetrahydrofuran-2-yl)pyrimidine-2,4(1H,3H)-dione

To a solution of 2′-O-methyluridine (56.0 g, 217 mmol) in CH₃CN (110 mL) and pyridine (450 mL), PPh₃ (73.94 g, 281.9 mmol), was added iodine (82.56 g, 325.3 mmol) in portions with a cold water bath. The reaction mixture was stirred at 25° C. under argon atmosphere for overnight. The reaction mixture was quenched by addition of saturated aqueous Na₂S₂O₃ (100 mL). The aqueous layer was extracted with EA (5×400 mL). The combined organic layers were dried over anhydrous Na₂SO₄. After filtration, the filtrate was concentrated under reduced pressure to afford title compound (132 g, 50% purity with 50% OPPh₃, 82% yield) as an off-white solid. The crude solid was used directly in the next step without further purification. MS(ESI): m/z=369.0 [M+H]⁺.

### Step 2: 1-((2R,3R,4S,5S)-4-((tert-butyldimethylsilyl)oxy)-5-(iodomethyl)-3-methoxytetrahydro furan-2-yl)pyrimidine-2,4(1H,3H)-dione

To a solution of 1-[(2R,3R,4S,5S)-4-hydroxy-5-(iodomethyl)-3-methoxytetrahydro-2-furyl]-1,2,3,4-tetra-hydropyrimidine-2,4-dione (130 g, 184 mmol) in DMF (500 mL) followed by the addition of imidazole (48.08 g, 706.2 mmol), DMAP (8.63 g, 70.6 mmol) and TBSCI (79.84 g, 530 mmol) in several portions. The reaction was stirred at room temperature for 3 hours. The reaction mixture was quenched by addition of cold water (50 mL). The aqueous layer was extracted with ethyl acetate (1×2 L) and washed with brine (500 mL). The combined organic layers were dried over anhydrous Na₂SO₄. After filtration, the filtrate was concentrated under reduced pressure. The crude product was precipitated from hexane. After filtration, crude white solid was collected to afford the titled compound (94 g, 50% purity with 50% OPPh₃, 90% yield) as an off-white solid. MS(ESI): m/z=483.1 [M+H]⁺.

### Step 3: 1-((2R,3R,4R,5R)-5-(azidomethyl)-4-((tert-butyldimethylsilyl)oxy)-3-methoxytetrahydro furan-2-yl)pyrimidine-2,4(1H,3H)-dione

To a solution of 1-((2R,3R,4S,5S)-4-((tert-butyldimethylsilyl)oxy)-5-(iodomethyl)-3-methoxy-tetrahydrofuran-2-yl)pyrimidine-2,4(1H,3H)-dione (10.0 g, 10.37 mmol, 50% purity) in DMF (100 mL) were added NaN₃ (2.7 g, 41.46 mmol), and the reaction was stirred at 50° C. for 3 hours. The reaction was quenched with sat. NaHCO₃ (aq, 50 mL), extracted with EA (2×200 mL). The combined organic layer was washed with brine (100 mL), dried over anhydrous Na₂SO₄. After filtration, the filtrate was concentrated under reduced pressure, purified by silica gel with EA/PE, to afford the titled compound (4.0 g, 97% yield) as a white solid. MS (ESI): m/z=398.2 [M+H]⁺.

### Step 4: 1-((2R,3R,4R,5R)-5-(aminomethyl)-4-((tert-butyldimethylsilyl)oxy)-3-methoxytetra-hydrofuran-2-yl)pyrimidine-2,4(1H,3H)-dione

To a solution of 1-((2R,3R,4R,5R)-5-(azidomethyl)-4-((tert-butyldimethylsilyl)oxy)-3-methoxy-tetrahydrofuran-2-yl)pyrimidine-2,4(1H,3H)-dione (3.6 g, 9.1 mmol) in THF (32.4 mL), H₂O (3.6 mL) were added PPh₃ (4.8 g, 18 mmol). The reaction was stirred at room temperature overnight. The reaction was quenched with water (50 mL) and extracted with DCM (2×200 mL). The organic layer was washed with brine (100 mL), dried over anhydrous Na₂SO₄. After filtration, the filtrate was concentrated under reduced pressure, purified by silica gel (MeOH/DCM) to afford the titled compound (2.5 g, 74% yield) as a white solid. MS (ESI): m/z=372.2 [M+H]⁺.

### Step 5 1-((2R,3R,4R,5S)-5-((bis(4-methoxyphenyl)(phenyl)methoxy)methyl)-4-(2-((((2R,3R,4R,5R)-3-((tert-butyldimethylsilyl)oxy)-5-(2,4-dioxo-3,4-dihydropyrimidin-1(2H)-yl)-4-methoxytetrahydrofuran-2-yl)methyl)amino)ethyl)-3-fluorotetrahydrofuran-2-yl)pyrimidine-2,4(1H,3H)-dione

To a solution of 1-((2R,3R,4R,5R)-5-(aminomethyl)-4-((tert-butyldimethylsilyl)oxy)-3-methoxy tetra-hydrofuran-2-yl)pyrimidine-2,4(1H,3H)-dione (1.8 g, 4.85 mmol) in DCM (32 mL) were added 2-((2S,3R,4R,5R)-2-((bis(4-methoxyphenyl)(phenyl)methoxy)methyl)-5-(2,4-dioxo-3,4-dihydropyrimidin-1(2H)-yl)-4-fluorotetrahydrofuran-3-yl)acetaldehyde (3.20 g, 5.57 mmol) and NaBH(OAc)₃ (1.34 g, 6.30 mmol). The reaction was stirred at room temperature for 2 hours under nitrogen atmosphere. The reaction was quenched with H₂O (10 mL). The crude was combined with another batch on the same scale. The crude mixture was extracted with EA (2×500 mL). The combined organic layers were washed with brine, dried over anhydrous Na₂SO₄. After filtration, the filtrate was concentrated under reduced pressure.

The residue was purified with silica gel (MeOH/DCM) to afford the titled compound (5.0 g, 74% purity, 52% yield) as a white solid. MS (ESI) m/z=930.3 [M+H]⁺.

### Step 6: methyl (2-((2S,3R,4R,5R)-2-((bis(4-methoxyphenyl)(phenyl)methoxy)methyl)-5-(2,4-dioxo-3,4-dihydropyrimidin-1(2H)-yl)-4-fluorotetrahydrofuran-3-yl)ethyl)(((2R,3R,4R,5R)-3-((tert-butyldimethylsilyl)oxy)-5-(2,4-dioxo-3,4-dihydropyrimidin-1(2H)-yl)-4-methoxytetrahydrofuran-2-yl)methyl)carbamodithioate

To a solution of 1-((2R,3R,4R,5S)-5-((bis(4-methoxyphenyl)(phenyl) methoxy)methyl)-4-(2-((((2R,3R,4R,5R)-3-((tert-butyldimethylsilyl)oxy)-5-(2,4-dioxo-3,4-dihydropyrimidin-1(2H)-yl)-4-methoxytetrahydrofuran-2-yl)methyl)amino)ethyl)-3-fluorotetrahydrofuran-2-yl)pyrimidine-2,4(1H,3H)-dione (2.5 g, 74% purity, 1.99 mmol) in DMF (25 mL) was added NaH (305 mg, 7.62 mmol, 60% in oil) at 0° C. The reaction mixture stirred at 0° C. for 30 min. To the mixture was added CS₂ (290 mg, 3.82 mmol) at 0° C. The reaction mixture stirred at room temperature overnight. Iodomethane (355 mg, 2.54 mmol) was added into the reaction mixture at 0° C. The reaction mixture stirred at room temperature for 3 hours. The reaction was quenched with sat. NaHCO₃ (aq, 50 mL) at 0° C., extracted with EA (3×500 mL). The combined organic layers were washed with brine, dried over sodium sulfate. After filtration, the filtrate was concentrated under reduced pressure. The residue was purified by column, C18; mobile phase, ACN in Water, 40% to 95% gradient in 30 min; UV 254 nm. To afford the titled compound (2.0 g, 85% purity, 80% yield) as a yellow solid. MS (ESI, negative mode): m/z=1018.5[M−H]⁻.

### Step 7: 1-((2R,3R,4R,5S)-5-((bis(4-methoxyphenyl)(phenyl)methoxy)methyl)-4-(2-((((2R,3R,4R,5R)-3-((tert-butyldimethylsilyl)oxy)-5-(2,4-dioxo-3,4-dihydropyrimidin-1(2H)-yl)-4-methoxytetra hydrofuran-2-yl)methyl)(trifluoromethyl)amino)ethyl)-3-fluorotetrahydrofuran-2-yl)pyrimidine-2,4(1H,3H)-dione

To a solution of methyl (2-((2S,3R,4R,5R)-2-((bis(4-methoxyphenyl)(phenyl) methoxy)methyl)-5-(2,4-dioxo-3,4-dihydropyrimidin-1(2H)-yl)-4-fluorotetrahydrofuran-3-yl)ethyl)(((2R,3R,4R,5R)-3-((tert-butyldimethylsilyl)oxy)-5-(2,4-dioxo-3,4-dihydropyrimidin-1(2H)-yl)-4-methoxytetrahydrofuran-2-yl)methyl)carbamodithioate (2.0 g, 1.96 mmol, 85% purity) in DCM (40 mL) were added triethylamine trihydrofluoride (2.9 g, 18.0 mmol) and 1,3-dibromo-5,5-dimethylhydantoin (1.03 g, 3.62 mmol) at −20° C., and the reaction was stirred at −20° C. for 1 hour. The reaction was quenched by NaHCO₃ (sat, 200 mL). The mixture was extracted with EA (3×500 mL). The combined layers were washed with brine (200 mL) and then dried over sodium sulfate. After filtration, the filtrate was concentrated and the residue was purified by C18; mobile phase, ACN in Water, 40% to 95% gradient in 30 min to afford the a mixture of titled compound, mono-Br of the titled compound and di-bromo of the titled compound as an inseparable mixture (1.6 g, 82% yield, calculated based on the product, ratio=3:2:1, product:mono-Br:di-Br) as a yellow solid. Titled compound, MS (ESI, negative mode): m/z=996.4 [M−H]⁻; Mono-Br product MS (ESI, negative mode): m/z=1074.3 [M−H]⁻; DiBr product MS (ESI, negative mode): m/z=1152.2 [M−H]⁻.

### Step 8: 1-((2R,3R,4R,5S)-5-((bis(4-methoxyphenyl)(phenyl)methoxy)methyl)-4-(2-((((2R,3R,4R,5R)-5-(2,4-dioxo-3,4-dihydropyrimidin-1(2H)-yl)-3-hydroxy-4-methoxytetra hydrofuran-2-yl)methyl)(trifluoromethyl) amino)ethyl)-3-fluorotetrahydrofuran-2-yl)pyrimidine-2,4(1H,3H)-dione

To a solution mixture of 1-((2R,3R,4R,5S)-5-((bis(4-methoxyphenyl)(phenyl) methoxy)methyl)-4-(2-((((2R,3R,4R,5R)-3-((tert-butyldimethylsilyl)oxy)-5-(2,4-dioxo-3,4-dihydropyrimidin-1(2H)-yl)-4-methoxytetrahydrofuran-2-yl)methyl) (trifluoromethyl)amino)ethyl)-3-fluorotetra-hydrofuran-2-yl)pyrimidine-2,4(1H,3H)-dione, and the corresponding mono-Br and di-Br compound (1.6 g, 1.6 mmol) in MeOH (72 mL), tetrahydrofuran (12 mL) were added Pd/C (1.6 g), and TEA (958 mg, 9.485 mmol). The reaction was stirred at room temperature overnight under hydrogen atmosphere at balloon pressure. The mixture was filtered and washed with MeOH, the filtrate was concentrated under reduced pressure. The residue was purified by column, C18; mobile phase, ACN in water, 10 to 95% gradient in 30 min; UV 254 nm to afford the titled compound (1.0 g, 84% purity, 84% yield) as a yellow solid. MS (ESI, negative mode) m/z=996.4 [M−H]⁻.

### Step 9: 1-((2R,3R,4R,5S)-5-((bis(4-methoxyphenyl)(phenyl)methoxy)methyl)-4-(2-((((2R,3R,4R,5R)-5-(2,4-dioxo-3,4-dihydropyrimidin-1(2H)-yl)-3-hydroxy-4-methoxytetrahydrofuran-2-yl)methyl) (trifluoromethyl) amino)ethyl)-3-fluorotetrahydrofuran-2-yl)pyrimidine-2,4(1H,3H)-dione

To a solution of 1-((2R,3R,4R,5S)-5-((bis(4-methoxyphenyl)(phenyl) methoxy)methyl)-4-(2-((((2R,3R,4R,5R)-3-((tert-butyldimethylsilyl)oxy)-5-(2,4-dioxo-3,4-dihydropyrimidin-1(2H)-yl)-4-methoxytetrahydrofuran-2-yl)methyl)(trifluoromethyl) amino)ethyl)-3-fluorotetrahydrofuran-2-yl)pyrimidine-2,4(1H,3H)-dione (1.0 g, 1.00 mmol, 84% purity) in DMSO (10 mL) were added CsF (305 mg, 2.01 mmol), the reaction was stirred at room temperature for overnight. The reaction was quenched by water (100 mL), extracted with EA (3×100 mL). The combined organic layers were washed brine (50 mL) and dried over sodium sulfate. The mixture was filtered, the concentrate was purified by column chromatography on silica gel (eluted with MeOH:DCM (contained 0.5% TEA)) to afford the titled compound (total: 800 mg, 60% purity, 70% yield) as a white solid. MS (ESI, negative mode): m/z=882.2 [M−H]⁻. Combined this product with another batch (total amount of crude product: 1.4 g). The product was further purified with SFC using the following condition: Column: CHIRAL ART Amylose-SA 3*25 cm, 5 um; Mobile Phase A: CO₂, Mobile Phase B: IPA:CH₃CN=3:1; Flow rate: 100 g/min; Gradient: 35% B in 13 min; Wave Length: 254 nm/220 nm, to afford the first eluent (Rt=1.874 min, analytical HPLC) as the titled compound (650 mg, 94% purity, de>95%) as a white solid. MS (ESI, negative mode): m/z=882.2 [M−H]⁻.

### Step 10: (2R,3R,4R,5R)-2-(((2-((2S,3R,4R,5R)-2-((bis(4-methoxyphenyl)(phenyl) methoxy)methyl)-5-(2,4-dioxo-3,4-dihydropyrimidin-1(2H)-yl)-4-fluorotetrahydrofuran-3-yl)ethyl)(trifluoromethyl) amino)methyl)-5-(2,4-dioxo-3,4-dihydropyrimidin-1(2H)-yl)-4-methoxytetrahydrofuran-3-yl(2-cyanoethyl) diisopropylphosphoramidite

To a mixture of 4,5-dicyanoimidazole (130 mg, 1.104 mmol), 4 A MS in DCM (13 mL) were added 2-cyanoethyl-N,N,N′,N′-tetraisopropylphosphorodiamidite (332 mg, 1.10 mmol). The reaction was stirred at room temperature for 10 minutes under nitrogen atmosphere, (2R,3R,4R,5R)-2-(((2-((2S,3R,4R,5R)-2-((bis(4-methoxyphenyl)(phenyl) methoxy) methyl)-5-(2,4-dioxo-3,4-dihydropyrimidin-1(2H)-yl)-4-fluorotetrahydrofuran-3-yl)ethyl)-(trifluoromethyl)amino)methyl)-5-(2,4-dioxo-3,4-dihydropyrimidin-1(2H)-yl)-4-methoxytetra-hydrofuran-3-yl(2-cyanoethyl) diisopropylphosphoramidite (650 mg, 0.736 mmol) was added dropwise at room temperature, the reaction was stirred at room temperature for overnight under nitrogen atmosphere. The reaction was diluted with DCM (contained 0.5% TEA) and filtered. The filtrate was washed with aqueous NaHCO₃ (sat, 100 mL), dried over magnesium sulfate. The mixture was filtered, the concentrate was purified by column chromatography on silica gel eluted with MeOH:DCM (0 to 10%, contained 1% TEA) to afford the title compound NB-200 (539 mg, 65.9% yield) as a white solid. ³¹P NMR (202 MHz, CDCl₃) δ 149.87, 149.56. MS (ESI): m/z=1106.5 [M+Na]⁺.

Oligonucleotide Comprising Dinucleotide NB-200 has been Synthesized Using the General Procedure Described in Example 1.

### Example 90: NB-201

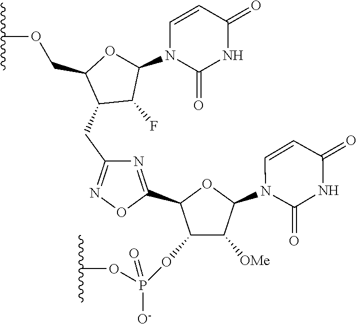 [SMILES: *OC[C@H]1O[C@@H](n2ccc(=O)[nH]c2=O)[C@H](F)[C@@H]1Cc1noc([C@H]2O[C@@H](n3ccc(=O)[nH]c3=O)[C@H](OC)[C@@H]2OP(=O)([O-])O*)n1]

### Synthesis of NB-201: (2S,3S,4R,5R)-2-(3-(((2S,3R,4R,5R)-2-((bis(4-methoxyphenyl)(phenyl)methoxy)methyl)-5-(2,4-dioxo-3,4-dihydropyrimidin-1(2H)-yl)-4-fluorotetrahydrofuran-3-yl)methyl)-1,2,4-oxadiazol-5-yl)-5-(2,4-dioxo-3,4-dihydropyrimidin-1(2H)-yl)-4-methoxytetrahydrofuran-3-yl (2-cyanoethyl) diisopropylphosphoramidite

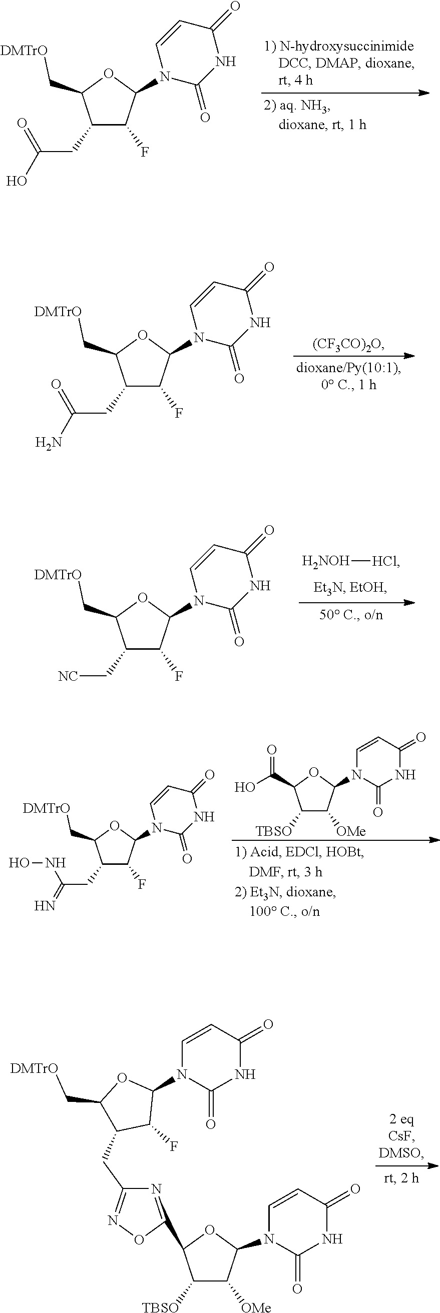

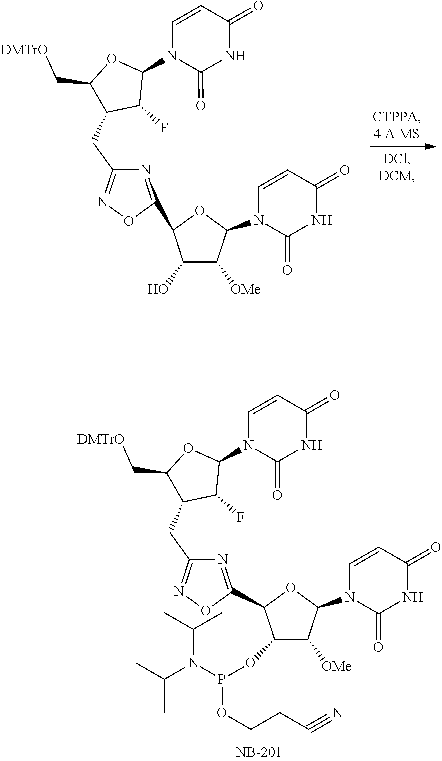

### Step 1: 2-((2S,3R,4R,5R)-2-((bis(4-methoxyphenyl)(phenyl)methoxy)methyl)-5-(2,4-dioxo-3,4-dihydro-pyrimidin-1(2H)-yl)-4-fluorotetrahydrofuran-3-yl)acetamide

To a stirred solution of 2-((2S,3R,4R,5R)-2-((bis(4-methoxyphenyl)(phenyl) methoxy)methyl)-5-(2,4-dioxo-3,4-dihydropyrimidin-1(2H)-yl)-4-fluorotetrahydrofuran-3-yl)acetic acid (9.0 g, 15.2 mmol) in dioxane (180 mL) and DMF (18 mL) were added DCC (4.09 g, 19.8 mmol), DMAP (0.56 g, 4.57 mmol) and N-hydroxysuccinimide (2.28 g, 19.8 mmol) in portions at room temperature. The resulting mixture was stirred for 4 hours at 25° C. under argon atmosphere. The reaction mixture was filtered. And the filter cake was washed with ACN. To the combined filtrates was added ammonium hydroxide (3.6 mL, 4.6 mmol) and the solution was allowed to stir for 10 min. The reaction mixture was diluted with EA (500 mL). The combined organic layers were washed with saturated NaHCO₃ solution (3×100 mL), brine (3×100 mL), dried by MgSO₄, filtered and concentrated to afford the titled compound (11 g, 94% yield) as a yellow solid. MS (ESI, negative mode): m/z=588.3 [M−H]⁺.

### Step 2: 2-((2S,3R,4R,5R)-2-((bis(4-methoxyphenyl)(phenyl)methoxy)methyl)-5-(2,4-dioxo-3,4-dihydro pyrimidin-1(2H)-yl)-4-fluorotetrahydrofuran-3-yl)acetonitrile

To a stirred solution of 2-((2S,3R,4R,5R)-2-((bis(4-methoxyphenyl)(phenyl) methoxy)methyl)-5-(2,4-dioxo-3,4-dihydropyrimidin-1(2H)-yl)-4-fluorotetrahydrofuran-3-yl)acetamide (10.0 g, 17 mmol) in dioxane (200 mL) and pyridine (20 mL) were added 2,2,2-trifluoroacetic anhydride (2.4 mL, 17.0 mmol) dropwise at 0° C. under argon atmosphere. The resulting mixture was stirred for 1 hour at 25° C. at argon atmosphere. The reaction mixture was diluted with ethyl acetate (600 mL). The combined organic layers were washed with saturated NaHCO₃ solution (2×100 mL) and brine (2×100 mL), dried by Na₂SO₄, filtered and concentrated. The residue was purified by silica gel column chromatography, eluted with MeOH:DCM (1:10) to afford the titled compound (7.0 g, 58% yield) as an off-white solid. MS (ESI, negative mode): m/z=570.3 [M−H]⁺.

### Step 3: 2-((2S,3R,4R,5R)-2-((bis(4-methoxyphenyl)(phenyl)methoxy)methyl)-5-(2,4-dioxo-3,4-dihydro-pyrimidin-1(2H)-yl)-4-fluorotetrahydrofuran-3-yl)-N-hydroxyacetimidamide

To a stirred solution of 2-((2S,3R,4R,5R)-2-((bis(4-methoxyphenyl)(phenyl) methoxy)methyl)-5-(2,4-dioxo-3,4-dihydropyrimidin-1(2H)-yl)-4-fluorotetrahydrofuran-3-yl)acetonitrile (6.0 g, 10.5 mmol) and triethylamine (2.9 mL, 21 mmol) in EtOH (60 mL) were added hydroxylamine hydrochloride (1.1 g, 16 mmol) in portions at 25° C. The resulting mixture was stirred overnight at 50° C. under argon atmosphere. The resulting mixture was concentrated under reduced pressure to afford the titled compound (6.0 g, 77% yield) as a white solid. MS (ESI, negative mode): m/z=603.3 [M−H]⁻. The product was used directly in the next step without further purification.

### Step 4: 1-((2R,3R,4R,5S)-5-((bis(4-methoxyphenyl)(phenyl)methoxy)methyl)-4-((5-((2S,3S,4R,5R)-3-((tert-butyldimethylsilyl)oxy)-5-(2,4-dioxo-3,4-dihydropyrimidin-1(2H)-yl)-4-methoxytetra-hydrofuran-2-yl)-1,2,4-oxadiazol-3-yl)methyl)-3-fluorotetrahydrofuran-2-yl)pyrimidine-2,4(1H,3H)-dione

To a solution of (2S,3S,4R,5R)-3-((tert-butyldimethylsilyl)oxy)-5-(2,4-dioxo-3,4-dihydropyrimidin-1(2H)-yl)-4-methoxytetrahydrofuran-2-carboxylic acid (3.84 g, 9.93 mmol) in DMF (30 mL) were added EDCI (3.80 g, 19.847 mmol) and HOBt (2.95 g, 21.8 mmol), and the reaction was stirred at room temperature for 20 minutes. To the above mixture was added 2-((2S,3R,4R,5R)-2-((bis(4-methoxyphenyl) (phenyl) methoxy)methyl)-5-(2,4-dioxo-3,4-dihydropyrimidin-1(2H)-yl)-4-fluorotetrahydrofuran-3-yl)-N-hydroxy-acetimidamide (6.0 g, 9.9 mmol) in DMF (30 mL) dropwise at 25° C. The resulting mixture was stirred for 3 hours at 25° C. under argon atmosphere. The reaction was quenched by the addition of saturated NaHCO₃ solution (100 mL) at 0° C. Extracted the aqueous layer with ethyl acetate (3×250 mL). The combined organic layers were washed with brine (3×100 mL), dried by Na₂SO₄, filtered and concentrated to obtain a solid. The solid was dissolved in dioxane (200 mL) and triethylamine (2.8 mL, 19.8 mmol). The resulting mixture was stirred overnight at 100° C. The reaction was cooled and quenched by the addition of saturated NaHCO₃ solution (250 mL) at 0° C. Extracted the aqueous layer with ethyl acetate (3×250 mL). The combined organic layers were washed with brine (3×100 mL), dried by Na₂SO₄, filtered and concentrated. The residue was purified using C18 column eluting with MeCN and H₂O (0%-95%, 30 min) to afford the title compound (4.2 g, 4.178 mmol, 42.10%) as a brown solid. MS(ESI, negative mode): m/z=953.4 [M−H]⁺.

### Step 5: 1-((2R,3R,4R,5S)-5-((bis(4-methoxyphenyl)(phenyl)methoxy)methyl)-4-((5-((2S,3S,4R,5R)-5-(2,4-dioxo-3,4-dihydropyrimidin-1(2H)-yl)-3-hydroxy-4-methoxytetrahydrofuran-2-yl)-1,2,4-oxadiazol-3-yl)methyl)-3-fluorotetrahydrofuran-2-yl)pyrimidine-2,4(1H,3H)-dione

To a stirred solution of 1-((2R,3R,4R,5S)-5-((bis(4-methoxyphenyl)(phenyl) methoxy)methyl)-4-((5-((2S,3S,4R,5R)-3-((tert-butyldimethylsilyl)oxy)-5-(2,4-dioxo-3,4-dihydropyrimidin-1(2H)-yl)-4-methoxy tetrahydrofuran-2-yl)-1,2,4-oxadiazol-3-yl)methyl)-3-fluorotetrahydrofuran-2-yl)pyrimidine-2,4(1H,3H)-dione (5.0 g, 5.2 mmol) in DMSO (50 mL) were added CsF (1.59 g, 10.5 mmol) in portions at room temperature. The resulting mixture was stirred for 3 hours at room temperature. The reaction was quenched by the addition of saturated NaHCO₃ solution (100 mL) at 0° C. Extracted the aqueous layer with ethyl acetate (3×250 mL). The combined organic layers were washed with brine (3×100 mL), dried by Na₂SO₄. filtered and concentrated. The residue was purified using C18 column eluting with MeCN and H₂O to afford the title compound (3.7 g, 80% yield) as a yellow solid. MS(ESI, negative mode): m/z=839.3 [M−H]⁺.

### Step 6: (2S,3S,4R,5R)-2-(3-(((2S,3R,4R,5R)-2-((bis(4-methoxyphenyl)(phenyl) methoxy)methyl)-5-(2,4-dioxo-3,4-dihydropyrimidin-1(2H)-yl)-4-fluorotetrahydrofuran-3-yl)methyl)-1,2,4-oxadiazol-5-yl)-5-(2,4-dioxo-3,4-dihydropyrimidin-1(2H)-yl)-4-methoxytetrahydrofuran-3-yl(2-cyanoethyl) diisopropylphosphoramidite

To a stirred suspension of cyanoethyl-N,N,N′,N′-tetraisopropylphosphorodiamidite (1.72 g, 5.71 mmol) and 4 A MS in DCM (64 mL) was added 4,5-dicyanoimidazole (0.67 g, 5.71 mmol) in portions at room temperature. The resulting mixture was stirred for 10 minutes at RT under argon atmosphere. To the above mixture was added 1-((2R,3R,4R,5S)-5-((bis(4-methoxyphenyl)(phenyl) methoxy)methyl)-4-((5-((2S,3S,4R,5R)-5-(2,4-dioxo-3,4-dihydropyrimidin-1(2H)-yl)-3-hydroxy-4-methoxy tetrahydrofuran-2-yl)-1,2,4-oxadiazol-3-yl)methyl)-3-fluorotetrahydrofuran-2-yl)pyrimidine-2,4(1H,3H)-dione (3.2 g, 3.806 mmol) in DCM (32 mL) dropwise at room temperature. The resulting mixture was stirred for 1 hour at room temperature. The reaction mixture was diluted with DCM (500 mL with 0.5% TEA). The reaction mixture was filtered and the filter cake was washed by DCM (300 mL with 0.5% TEA). The combined organic layers were washed with saturated NaHCO₃ solution (3×100 mL) and brine (3×100 mL), dried by MgSO₄, filtered and concentrated. The residue was purified by silica gel column chromatography, eluted with MeOH:DCM (contained 1% TEA) (1:10) to afford the titled compound NB-201 (3.1 g, 76% yield) as an off-white solid. ³¹P NMR (202 MHz, DMSO-d₆) δ 151.43, 150.84. MS (ESI, negative mode): 1039.3 [M−H]⁻.

### Oligonucleotide Comprising Dinucleotide NB-201 to be Synthesized Using the General Procedure Described in Example 1.

### Example 91: NB-202

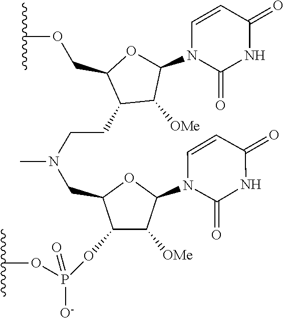 [SMILES: *OC[C@H]1O[C@@H](n2ccc(=O)[nH]c2=O)[C@H](OC)[C@@H]1CCN(C)C[C@H]1O[C@@H](n2ccc(=O)[nH]c2=O)[C@H](OC)[C@@H]1OP(=O)([O-])O*]

### Synthesis of NB-202: (2R,3R,4R,5R)-2-(((2-((2S,3R,4R,5R)-2-((bis(4-methoxyphenyl)(phenyl)methoxy)methyl)-5-(2,4-dioxo-3,4-dihydropyrimidin-1(2H)-yl)-4-methoxytetrahydrofuran-3-yl)ethyl)(methyl)amino)methyl)-5-(2,4-dioxo-3,4-dihydropyrimidin-1(2H)-yl)-4-methoxytetrahydrofuran-3-yl (2-cyanoethyl) diisopropylphosphoramidite

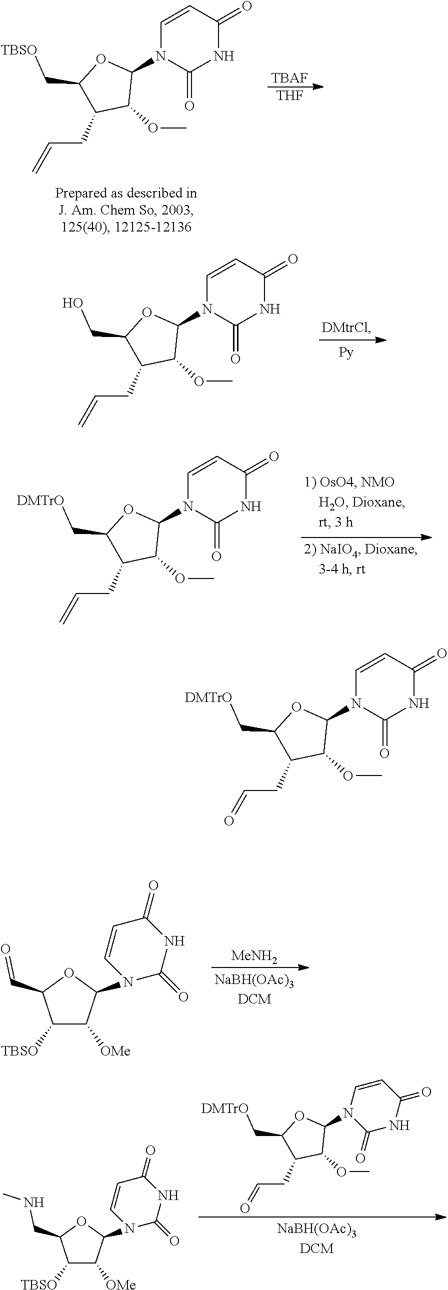

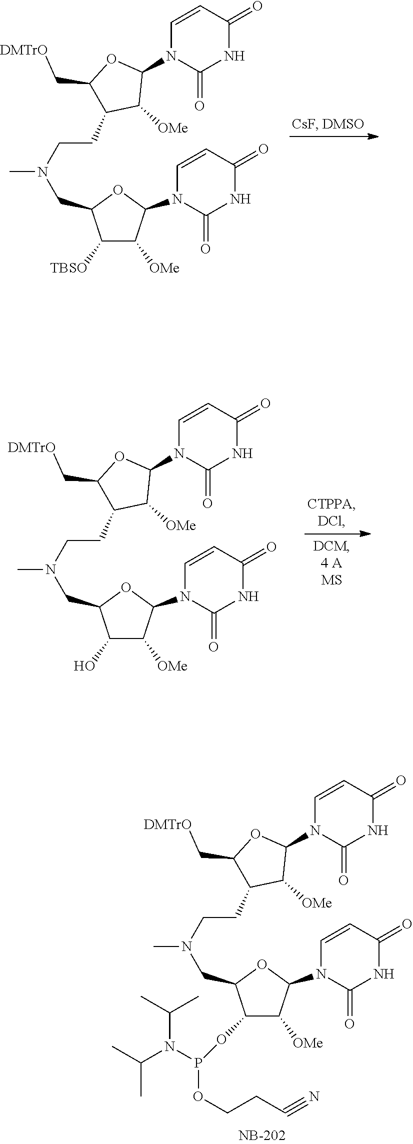

### Step 1: 1-((2R,3R,4R,5S)-4-allyl-5-(hydroxymethyl)-3-methoxytetrahydrofuran-2-yl)pyrimidine-2,4(1H,3H)-dione

To a stirred solution of 1-((2R,3R,4R,5S)-4-allyl-5-(((tert-butyldimethylsilyl)oxy)methyl)-3-methoxytetra hydrofuran-2-yl)pyrimidine-2,4(1H,3H)-dione (20.0 g, 50.43 mmol) in THF (200 mL) were added TBAF (1.0 M in THF, 50 mL, 50 mmol) at room temperature. The resulting mixture was stirred for 2 hours at room temperature under nitrogen atmosphere. The resulting mixture was concentrated under reduced pressure and was diluted with ethyl acetate (2 L). The combined organic layers were washed with water (5×100 mL) and brine (2×100 mL), dried by MgSO₄, filtered and concentrated to afford the titled compound (12.5 g, 87.8% yield) as an off-white solid which was used in the next step directly without further purification. MS(ESI): m/z=283.1 [M+H]⁺.

### Step 2: 1-((2R,3R,4R,5S)-4-allyl-5-((bis(4-methoxyphenyl)(phenyl)methoxy)methyl)-3-methoxytetrahydro furan-2-yl)pyrimidine-2,4(1H,3H)-dione

To a stirred solution of 1-((2R,3R,4R,5S)-4-allyl-5-(hydroxymethyl)-3-methoxytetrahydrofuran-2-yl) pyrimidine-2,4(1H,3H)-dione (12.5 g, 44.3 mmol) in pyridine (100 mL) were added DMT-Cl (22.50 g, 66.4 mmol) in portions at 0° C. The resulting mixture was stirred overnight at room temperature under N2 atmosphere. The reaction was quenched by the addition of MeOH (30 mL) at RT. The resulting mixture was concentrated under reduced pressure. The reaction mixture was diluted with ethyl acetate (1.5 L). The combined organic layers were washed with saturated NaHCO₃ solution (4×100 mL) and brine (3×50 mL), dried by Na₂SO₄, filtered and concentrated. The residue was purified by silica gel column chromatography, eluted with PE (0.5% TEA):EA (1:1) to afford the titled compound (20.5 g, 79.2% yield) as a yellow solid. MS (ESI): m/z=585.2 [M+H]⁺.

### Step 3: 2-((2S,3R,4R,5R)-2-((bis(4-methoxyphenyl)(phenyl)methoxy)methyl)-5-(2,4-dioxo-3,4-dihydro pyrimidin-1(2H)-yl)-4-methoxytetrahydrofuran-3-yl)acetaldehyde

To a stirred solution of 1-((2R,3R,4R,5S)-4-allyl-5-((bis(4-methoxyphenyl)(phenyl)methoxy) methyl)-3-methoxytetrahydrofuran-2-yl)pyrimidine-2,4(1H,3H)-dione (18.5 g, 31.6 mmol) in dioxane (190 mL) were added NMO (5.19 g, 44.3 mmol) and OsO₄ (19 mL, 1.27 mmol, 500 mg in water (30 mL)) at room temperature. The resulting mixture was protected from light and stirred for 3 hours at room temperature under nitrogen atmosphere. The reaction was quenched by the addition of saturated NaHCO₃ solution (100 mL). The resulting mixture was extracted with DCM (500 mL×2). The combined organic layers were washed with saturated NaHCO₃ solution (100 mL) and brine (100 mL), dried by Na₂SO₄, filtered and concentrated. The residue was dissolved in dioxane (190 mL). To the above mixture was added NaIO₄ (8.12 g, 38.0 mmol) in H₂O (56 mL) dropwise at room temperature. The resulting mixture was stirred for 3 hours at room temperature. The reaction was quenched by the addition of saturated NaHCO₃ solution (50 mL). The reaction mixture was extracted with ethyl acetate (500 mL×2). The combined organic layers were washed with saturated NaHCO₃ (aq., 100 mL), saturated Na₂S₂O₃ solution (100 mL), brine (50 mL), dried by Na₂SO₄, filtered and concentrated to afford the titled compound (19 g, 100% yield) as a light yellow solid. MS(ESI) m/z=587.2 [M+H]⁺. The crude product was used in the next step directly without further purification.

### Step 4: 1-((2R,3R,4R,5R)-4-((tert-butyldimethylsilyl)oxy)-3-methoxy-5-((methylamino) methyl)tetrahydrofuran-2-yl)pyrimidine-2,4(1H,3H)-dione

To a stirred solution of (2S,3S,4R,5R)-3-((tert-butyldimethylsilyl)oxy)-5-(2,4-dioxo-3,4-dihydropyrimidin-1(2H)-yl)-4-methoxytetrahydrofuran-2-carbaldehyde (12 g, 32 mmol) and methanamine (30% in water, 100 mL, 675 mmol) in MeOH (120 mL) were added NaBH(OAc)₃ (68.32 g, 323.9 mmol) in portion at 0° C. The resulting mixture was stirred for 2 hours at 25° C. at argon atmosphere. The reaction was quenched by the addition of saturated NaHCO₃ solution (250 mL) at 0° C. The resulting mixture was extracted with ethyl acetate (3×800 mL). The combined organic layer was washed with brine (250 mL), dried by Na₂SO₄, filtered and concentrated. The residue was purified by silica gel column chromatography, eluted with MeOH:DCM (1:4) to afford the titled compound (3.5 g, 26% yield) as a light yellow solid. MS(ESI) m/z=386.2 [M+H]⁺.

### Step 6: 1-((2R,3R,4R,5S)-5-((bis(4-methoxyphenyl)(phenyl)methoxy)methyl)-4-(2-((((2R,3R,4R,5R)-3-((tert-butyldimethylsilyl)oxy)-5-(2,4-dioxo-3,4-dihydropyrimidin-1(2H)-yl)-4-methoxytetrahydrofuran-2-yl)methyl)(methyl)amino)ethyl)-3-methoxytetrahydrofuran-2-yl)pyrimidine-2,4-(1H,3H)-dione

To a stirred solution of 2-((2S,3R,4R,5R)-2-((bis(4-methoxyphenyl)(phenyl) methoxy)methyl)-5-(2,4-dioxo-3,4-dihydropyrimidin-1(2H)-yl)-4-methoxytetrahydrofuran-3-yl)acetaldehyde (4.87 g, 8.30 mmol), 1-((2R,3R,4R,5R)-4-((tert-butyldimethylsilyl)oxy)-3-methoxy-5-((methylamino)methyl)tetrahydrofuran-2-yl)pyrimidine-2,4(1H,3H)-dione (3.2 g, 8.3 mmol) in DCM (64 mL), was added NaBH(OAc)₃ (2.11 g, 9.96 mmol) at room temperature. The resulting mixture was stirred for 2 hours at room temperature under nitrogen atmosphere. The reaction was quenched with H₂O (20 mL) and extracted with ethyl acetate (100 mL×2). The combined organic layers were washed brine (50 mL) and dried over sodium sulfate. The mixture was filtered and concentrated. The residue was purified by silica gel column chromatography eluted with MeOH:DCM (with 0.5% TEA) to afford the title compound (7.0 g, 80% purity, 77% yield) as a yellow solid, LCMS(ESI) m/z=956 [M+H]⁺.

### Step 7: 1-((2R,3R,4R,5S)-5-((bis(4-methoxyphenyl)(phenyl)methoxy)methyl)-4-(2-((((2R,3R,4R,5R)-5-(2,4-dioxo-3,4-dihydropyrimidin-1(2H)-yl)-3-hydroxy-4-methoxytetrahydrofuran-2-yl)methyl) (methyl)amino) ethyl)-3-methoxytetrahydrofuran-2-yl)pyrimidine-2,4(1H,3H)-dione

To a stirred solution of 1-((2R,3R,4R,5S)-5-((bis(4-methoxyphenyl)(phenyl)methoxy)methyl)-4-(2-((((2R,3R,4R,5R)-3-((tert-butyldimethylsilyl)oxy)-5-(2,4-dioxo-3,4-dihydropyrimidin-1(2H)-yl)-4-methoxy tetrahydrofuran-2-yl)methyl)(methyl)amino)ethyl)-3-methoxytetrahydrofuran-2-yl)pyrimidine-2,4(1H,3H)-dione (7.0 g, 7.3 mmol) in DMSO (70 mL) were added CsF (2.23 g, 14.6 mmol) at RT. The resulting mixture was stirred for overnight at room temperature under nitrogen atmosphere. The reaction was quenched with H₂O (100 mL), extracted with ethyl acetate (1 L×2). The combined organic layers were washed with brine (100 mL), dried over sodium sulfate, filtered and concentrated. The residue was purified by C18 column (ACN/H₂O) to afford the titled compound (4.5 g, 73% yield) as a yellow solid. MS(ESI): m/z=842.0, [M+H]⁺.

### Step 8: (2R,3R,4R,5R)-2-(((2-((2S,3R,4R,5R)-2-((bis(4-methoxyphenyl)(phenyl)methoxy) methyl)-5-(2,4-dioxo-3,4-dihydropyrimidin-1(2H)-yl)-4-methoxytetrahydrofuran-3-yl)ethyl)(methyl)amino)methyl)-5 (2,4-dioxo-3,4-dihydropyrimidin-1(2H)-yl)-4-methoxytetrahydro-furan-3-yl(2-cyanoethyl) diisopropylphosphoramidite

To a solution of 4,5-dicyanoimidazole (0.84 g, 7.127 mmol), 4 A MS in DCM (40 mL) were added 2-cyanoethyl N,N,N′,N′-tetraisopropylphosphorodiamidite (2.15 g, 7.127 mmol) the reaction was stirred at room temperature for 10 minutes under argon atmosphere, 1-((2R,3R,4R,5S)-5-((bis(4-methoxy phenyl)(phenyl)methoxy)methyl)-4-(2-((((2R,3R,4R,5R)-5-(2,4-dioxo-3,4-dihydropyrimidin-1(2H)-yl)-3-hydroxy-4-methoxytetrahydrofuran-2-yl)methyl)(methyl)amino)ethyl)-3-methoxytetrahydrofuran-2-yl)pyrimidine-2,4(1H,3H)-dione (4.0 g, 4.6 mmol) in DCM (40 mL), was added dropwise at RT. The reaction was stirred at room temperature for 1 hour under argon atmosphere. The reaction was diluted with DCM (500 mL, with 0.5% TEA) and filtered. The filtrate was washed with aqueous NaHCO₃ (sat, 100 mL), dried over magnesium sulfate, filtered and concentrated. The residue was purified by column chromatography on silica gel eluting with MeOH:DCM (1% TEA) to afford the title compound NB-202 (3.5 g, 70% yield) as a white solid. ³¹P NMR (202 MHz, DMSO-d₆) δ 149.75, 149.34. MS (ESI): 1042.5 [M+H]⁺ 1064.6[M+Na]⁺.

Oligonucleotide Comprising Dinucleotide NB-202 has been Synthesized Using the General Procedure Described in Example 1.

### Example 92: NB-203

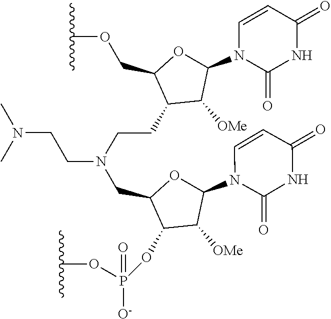 [SMILES: *OC[C@H]1O[C@@H](n2ccc(=O)[nH]c2=O)[C@H](OC)[C@@H]1CCN(CCN(C)C)C[C@H]1O[C@@H](n2ccc(=O)[nH]c2=O)[C@H](OC)[C@@H]1OP(=O)([O-])O*]

### Synthesis of NB-203: (2R,3R,4R,5R)-2-(((2-((2S,3R,4R,5R)-2-((bis(4-methoxyphenyl)(phenyl)methoxy) methyl)-5-(2,4-dioxo-3,4-dihydropyrimidin-1(2H)-yl)-4-methoxytetrahydrofuran-3-yl)ethyl)(2-(dimethyl-amino)ethyl) amino)methyl)-5-(2,4-dioxo-3,4-dihydropyrimidin-1(2H)-yl)-4-methoxytetrahydro-furan-3-yl (2-cyanoethyl)diisopropylphosphoramidite

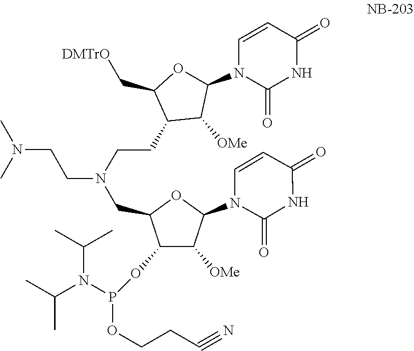

The synthesis of NB-203 followed the same procedure as in NB-202 by replacing the methylamine by N,N-dimethylethylenediamine to afford the titled compound NB-203 as a white solid. ³¹P NMR (202 MHz, DMSO-d₆) δ 149.38, 149.27. MS (ESI): 1099.6 [M+H]⁺.

Oligonucleotide Comprising Dinucleotide NB-203 has been Synthesized Using the General Procedure Described in Example 1.

### Example 93: NB-204

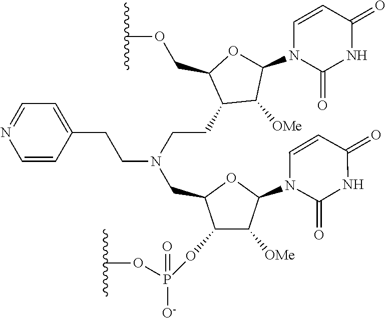 [SMILES: *OC[C@H]1O[C@@H](n2ccc(=O)[nH]c2=O)[C@H](OC)[C@@H]1CCN(CCc1ccncc1)C[C@H]1O[C@@H](n2ccc(=O)[nH]c2=O)[C@H](OC)[C@@H]1OP(=O)([O-])O*]

### Synthesis of NB-204: (2R,3R,4R,5R)-2-(((2-((2S,3R,4R,5R)-2-((bis(4-methoxyphenyl)(phenyl)methoxy) methyl)-5-(2,4-dioxo-3,4-dihydropyrimidin-1(2H)-yl)-4-methoxytetrahydrofuran-3-yl)ethyl)(2-(pyridin-4-yl)ethyl)amino) methyl)-5-(2,4-dioxo-3,4-dihydropyrimidin-1(2H)-yl)-4-methoxytetrahydrofuran-3-yl(2-cyanoethyl) diisopropylphosphoramidite

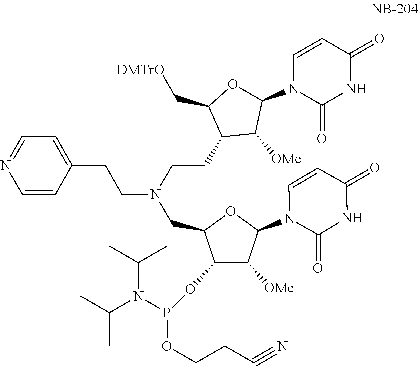

The synthesis of NB-204 followed the same procedure as in NB-202 by replacing the methylamine by 4-pyridineethanamine to afford the titled NB-204 compound as a white solid. ³¹P NMR (202 MHz, DMSO-d₆) δ 149.41, 149.25. MS (ESI): 1133.6 [M+H]⁺; 1155.8 [M+Na]⁺.

Oligonucleotide Comprising Dinucleotide NB-204 has been Synthesized Using the General Procedure Described in Example 1.

### Example 94: NB-205

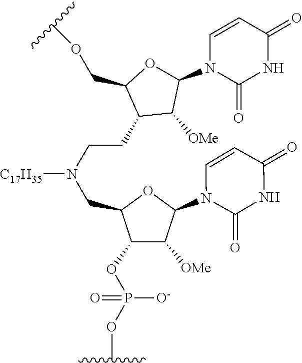

### Synthesis of NB-205: (2R,3R,4R,5R)-2-(((2-((2S,3R,4R,5R)-2-((bis(4-methoxyphenyl)(phenyl)methoxy)methyl)-5-(2,4-dioxo-3,4-dihydropyrimidin-1(2H)-yl)-4-methoxytetrahydrofuran-3-yl)ethyl)(heptadecyl)amino)methyl)-5-(2,4-dioxo-3,4-dihydropyrimidin-1(2H)-yl)-4-methoxytetrahydrofuran-3-yl(2-cyanoethyl) diisopropylphosphoramidite

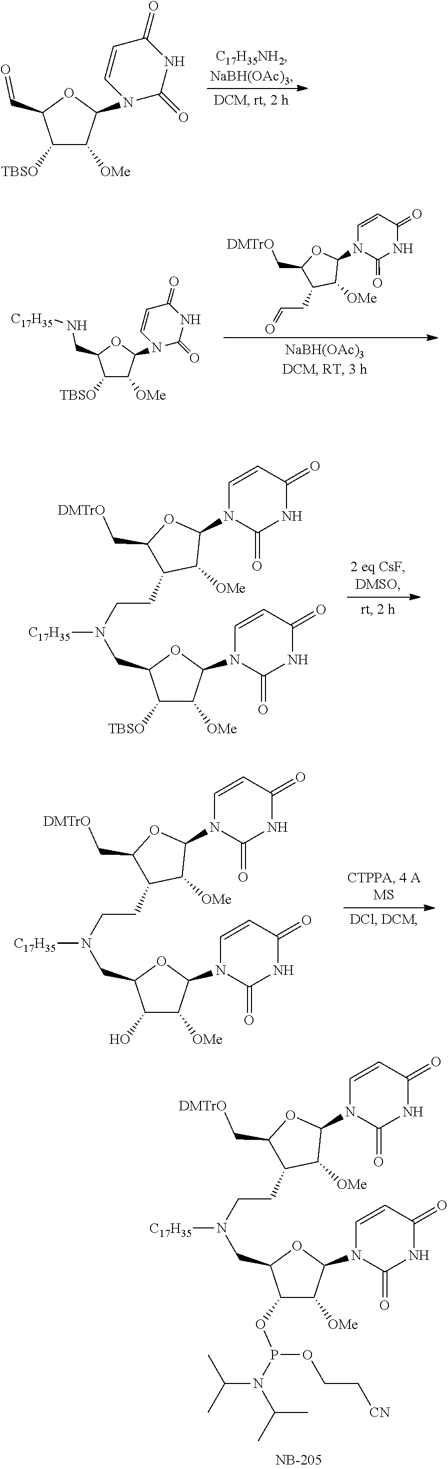

### Step 1: 1-((2R,3R,4R,5R)-4-((tert-butyldimethylsilyl)oxy)-5-((heptadecylamino)methyl)-3-methoxytetra-hydrofuran-2-yl)pyrimidine-2,4(1H,3H)-dione

To a stirred solution of (2S,3S,4R,5R)-3-((tert-butyldimethylsilyl)oxy)-5-(2,4-dioxo-3,4-dihydro pyrimidin-1(2H)-yl)-4-methoxytetrahydrofuran-2-carbaldehyde (4.50 g, 12.1 mmol) in DCM (90 mL) were added heptadecan-1-amine (3.10 g, 12.1 mmol), NaBH(OAc)₃ (3.09 g, 14.6 mmol) at room temperature. The resulting mixture was stirred for 2 hours at room temperature under nitrogen atmosphere. The reaction was quenched with H₂O (20 mL), extracted with EA (3×200 mL) and dried over sodium sulfate. The mixture was filtered and concentrated, purified by silica gel (MeOH/DCM) to afford the titled compound (5.0 g, 49% yield) as a yellow oil. MS(ESI): m/z=610.0 [M+H]⁺.

### Step 2: 1-((2R,3R,4R,5S)-5-((bis(4-methoxyphenyl)(phenyl)methoxy)methyl)-4-(2-((((2R,3R,4R,5R)-3-((tert-butyldimethylsilyl)oxy)-5-(2,4-dioxo-3,4-dihydropyrimidin-1(2H)-yl)-4-methoxytetra hydrofuran-2-yl)methyl)(heptadecyl)amino)ethyl)-3-methoxytetrahydrofuran-2-yl)pyrimidine-2,4(1H,3H)-dione

To a stirred solution of 1-((2R,3R,4R,5R)-4-((tert-butyldimethylsilyl)oxy)-5-((heptadecylamino) methyl)-3-methoxytetrahydrofuran-2-yl)pyrimidine-2,4(1H,3H)-dione (4.50 g, 7.38 mmol) in DCM (45 mL) were added 2-((2S,3R,4R,5R)-2-((bis(4-methoxyphenyl)(phenyl)methoxy)methyl)-5-(2,4-dioxo-3,4-dihydro-pyrimidin-1(2H)-yl)-4-methoxytetrahydrofuran-3-yl)-acetaldehyde (3.90 g, 6.64 mmol), NaBH(OAc)₃ (1.88 g, 8.85 mmol) at RT. The resulting mixture was stirred for 3 hours under nitrogen atmosphere. The reaction was quenched with H₂O (20 mL), extracted with EA (3×100 mL), dried over sodium sulfate. The mixture was filtered and concentrated. The residue was purified by silica gel (eluted with DCM/EA), to afford the titled compound (3.0 g, yield: 39%) as a white solid. MS(ESI): m/z=1181.4 [M+H]⁺.

### Step 3: 1-((2R,3R,4R,5S)-5-((bis(4-methoxyphenyl)(phenyl)methoxy)methyl)-4-(2-((((2R,3R,4R,5R)-5-(2,4-dioxo-3,4-dihydropyrimidin-1(2H)-yl)-3-hydroxy-4-methoxytetrahydrofuran-2-yl)methyl)(heptadecyl) amino)ethyl)-3-methoxytetrahydrofuran-2-yl)pyrimidine-2,4(1H,3H)-dione

To a stirred solution of 1-((2R,3R,4R,5S)-5-((bis(4-methoxyphenyl)(phenyl )methoxy)methyl)-4-(2-((((2R,3R,4R,5R)-3-((tert-butyldimethylsilyl)oxy)-5-(2,4-dioxo-3,4-dihydropyrimidin-1(2H)-yl)-4-methoxy tetrahydrofuran-2-yl)methyl)(heptadecyl) amino)ethyl)-3-methoxytetrahydrofuran-2-yl)pyrimidine-2,4(1H,3H)-dione (3.0 g, 2.54 mmol) in DMSO (30 mL) were added CsF (0.77 g, 5.08 mmol) at RT. The resulting mixture was stirred overnight at RT under nitrogen atmosphere. The reaction was quenched with H₂O (100 mL), extracted with EA (3×200 mL). The combined organic layer was washed with H₂O (3×200 mL), dried over sodium sulfate, filtered and concentrated. The residue was purified by silica gel (eluted with MeOH/DCM) to afford the titled compound (2.50 g, 92.2% yield) as a yellow solid. MS(ESI) m/z=1066.7 [M+H]⁺.

### Step 4: (2R,3R,4R,5R)-2-(((2-((2S,3R,4R,5R)-2-((bis(4-methoxyphenyl)(phenyl)methoxy)methyl)-5-(2,4-dioxo-3,4-dihydropyrimidin-1(2H)-yl)-4-methoxytetrahydrofuran-3-yl)ethyl)(heptadecyl) amino)methyl)-5-(2,4-dioxo-3,4-dihydropyrimidin-1(2H)-yl)-4-methoxytetrahydrofuran-3-yl(2-cyanoethyl) diisopropylphosphoramidite

To a suspension of 4,5-dicyanoimidazole (0.33 g, 2.81 mmol), 4 A MS in DCM (20 mL) were added 2-cyanoethyl N,N,N′,N′-tetraisopropylphosphorodiamidite (0.85 g, 2.81 mmol), the reaction was stirred at room temperature for 10 min under argon atmosphere, 1-((2R,3R,4R,5S)-5-((bis(4-methoxyphenyl) (phenyl)methoxy)methyl)-4-(2-((((2R,3R,4R,5R)-5-(2,4-dioxo-3,4-dihydropyrimidin-1(2H)-yl)-3-hydroxy-4-methoxytetrahydrofuran-2-yl)methyl)(heptadecyl)amino)ethyl)-3-methoxytetrahydrofuran-2-yl) pyrimidine-2,4(1H,3H)-dione (2.0 g, 1.9 mmol) in DCM (20 mL) was added dropwise at room temperature, the reaction was stirred at room temperature for 1 hour under argon atmosphere. The reaction was diluted with DCM (0.5% TEA) and was filtered. The filtrate was washed with saturated aqueous NaHCO₃ (100 mL). The organic layer was dried over magnesium sulfate, filtered and concentrated. The residue was purified by column chromatography on silica gel MeOH: DCM (1% TEA) to afford the title compound NB-205 (1.74 g, 73.5% yield) as an off-white solid. ³¹P NMR (162 MHz, DMSO-d₆) δ 149.10, 148.78. MS(ESI) m/z=1267.1 [M+H]⁺.

Oligonucleotide Comprising Dinucleotide NB-205 has been Synthesized Using the General Procedure Described in Example 1.

### Example 95: NB-206

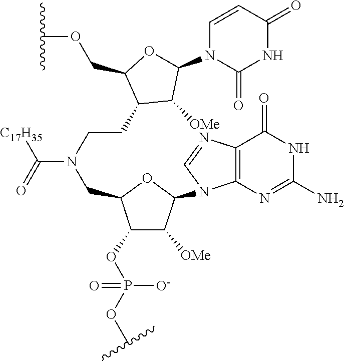

### Synthesis of NB-206: (2R,3R,4R,5R)-2-((N-(2-((2S,3R,4R,5R)-2-((bis(4-methoxyphenyl)(phenyl)methoxy)methyl)-5-(2,4-dioxo-3,4-dihydropyrimidin-1(2H)-yl)-4-methoxytetrahydrofuran-3-yl)ethyl)stearamido)methyl)-5-(2-iso-butyramido-6-oxo-1,6-dihydro-9H-purin-9-yl)-4-methoxytetrahydrofuran-3-yl(2-cyanoethyl) diisopropylphosphoramidite

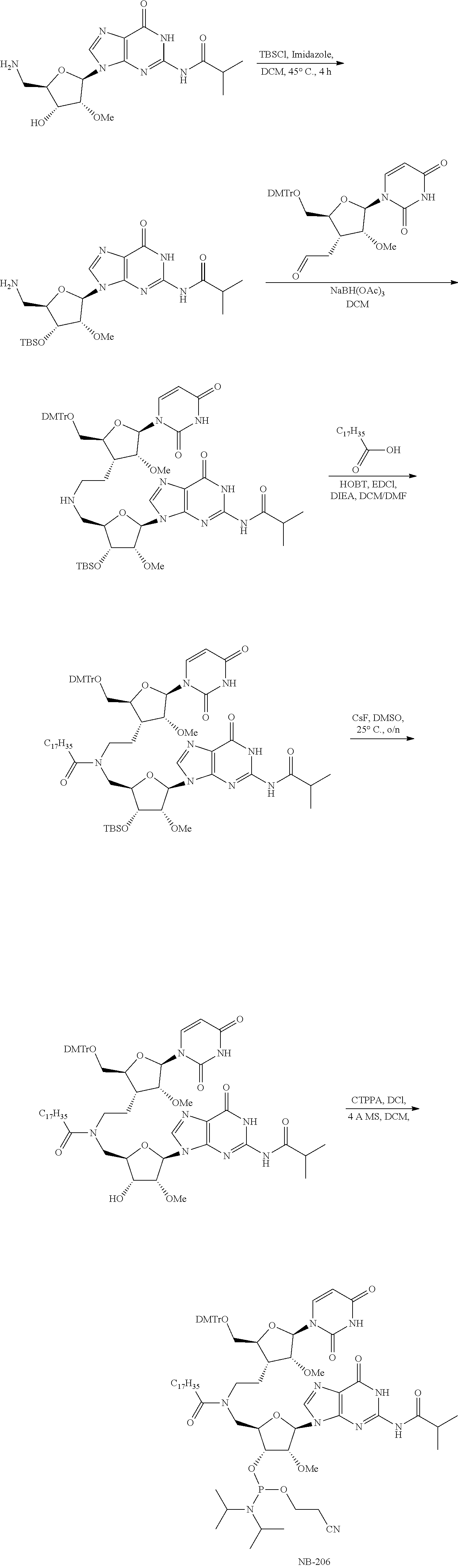

### Step 1: N-(9-((2R,3R,4R,5R)-5-(aminomethyl)-4-((tert-butyldimethylsilyl)oxy)-3-methoxytetra hydrofuran-2-yl)-6-oxo-6,9-dihydro-1H-purin-2-yl)isobutyramide

Added tert-butyldimethylsilyl chloride (5.76 g, 38.2 mmol) into a solution of N-(9-((2R,3R,4R,5R)-5-(aminomethyl)-4-hydroxy-3-methoxytetrahydrofuran-2-yl)-6-oxo-6,9-dihydro-1H-purin-2-yl)isobutyr-amide (3.50 g, 9.55 mmol) and imidazole (3.90 g, 57.3 mmol) in dichloromethane (35 mL). The reaction mixture stirred at 45° C. for 4 hours. The reaction was quenched with H₂O (10 mL). The resulting mixture was extracted with EA (2×100 mL), dried over sodium sulfate. The mixture was filtered and concentrated, purified by C18 (ACN/H₂O) to afford the titled compound (4.0 g, 76% yield) as a yellow oil. MS (ESI): m/z=481.0 [M+H]⁺.

### Step 2: N-(9-((2R,3R,4R,5R)-5-(((2-((2S,3R,4R,5R)-2-((bis(4-methoxyphenyl)(phenyl)methoxy) methyl)-5-(2,4-dioxo-3,4-dihydropyrimidin-1(2H)-yl)-4-methoxytetrahydrofuran-3-yl)ethyl) amino)methyl)-4-((tert-butyldimethylsilyl)oxy)-3-methoxytetrahydrofuran-2-yl)-6-oxo-6,9-dihydro-1H-purin-2-yl)isobutyramide

To a stirred solution of 2-((2S,3R,4R,5R)-2-((bis(4-methoxyphenyl)(phenyl) methoxy)methyl)-5-(2,4-dioxo-3,4-dihydropyrimidin-1(2H)-yl)-4-methoxytetrahydrofuran-3-yl)acetaldehyde (4.03 g, 6.86 mmol) and N-(9-((2R,3R,4R,5R)-5-(aminomethyl)-4-((tert-butyldimethylsilyl)oxy)-3-methoxytetrahydrofuran-2-yl)-6-oxo-6,9-dihydro-1H-purin-2-yl)isobutyramide (3.0 g, 6.2 mmol) in DCM (30 mL) were added NaBH(OAc)₃ (2.63 g, 12.5 mmol) in portions at RT. The resulting mixture was stirred for 2 hours at RT under argon atmosphere. The reaction was quenched by the addition of aqueous saturated NaHCO₃ (10 mL) solution at 0° C. and extracted the resulting mixture with EA (3×50 mL). The organic layers were washed with brine (3×20 mL), dried by Na₂SO₄. After filtration, the filtrate was concentrated under reduced pressure. The residue was purified by silica gel column chromatography, eluted with MeOH:DCM (0.5% TEA) (1:10) to afford the titled compound (3.8 g, 52% yield) as a white solid. MS(ESI) m/z=1052.6 [M+H]⁺.

### Step 3: N-(2-((2S,3R,4R,5R)-2-((bis(4-methoxyphenyl)(phenyl)methoxy)methyl)-5-(2,4-dioxo-3,4-dihydro pyrimidin-1(2H)-yl)-4-methoxytetrahydrofuran-3-yl)ethyl)-N-(((2R,3R,4R,5R)-3-((tert-butyl-dimethylsilyl) oxy)-5-(2-isobutyramido-6-oxo-1,6-dihydro-9H-purin-9-yl)-4-methoxytetrahydrofuran-2-yl) methyl)stearamide

To a stirred solution of N-(9-((2R,3R,4R,5R)-5-(((2-((2S,3R,4R,5R)-2-((bis(4-methoxyphenyl)(phenyl) methoxy)methyl)-5-(2,4-dioxo-3,4-dihydropyrimidin-1(2H)-yl)-4-methoxytetrahydrofuran-3-yl)ethyl) amino)methyl)-4-((tert-butyldimethylsilyl)oxy)-3-methoxytetrahydrofuran-2-yl)-6-oxo-6,9-dihydro-1H-purin-2-yl)isobutyramide (3.50 g, 3.33 mmol) and octadecanoic acid (0.95 g, 3.33 mmol) in DCM (27 mL) were added HOBt (0.67 g, 4.99 mmol), EDCI (0.96 g, 4.99 mmol) and DIEA (2.15 g, 16.646 mmol) batchwise at 0° C. under N2 atmosphere. The resulting mixture was stirred overnight at room temperature under N2 atmosphere. The reaction was quenched by the addition of aqueous saturated NaHCO₃ (10 mL) at room temperature. The resulting mixture was extracted with DCM (2×100 mL). The combined organic layers were washed with water (20 mL) and brine (20 mL), dried by Na₂SO₄. After filtration, the filtrate was concentrated under reduced pressure. The crude material was subjected to a silica gel column (eluted with 1:10 MeOH/DCM) and repurified with silica gel column (100% EA) to obtain the titled compound, (3.0 g, 68% yield) as a yellow solid. MS(ESI, negative mode) m/z=1315.9 [M−H]⁻.

### Step 4: N-(2-((2S,3R,4R,5R)-2-((bis(4-methoxyphenyl)(phenyl)methoxy)methyl)-5-(2,4-dioxo-3,4-dihydro pyrimidin-1(2H)-yl)-4-methoxytetrahydrofuran-3-yl)ethyl)-N-(((2R,3R,4R,5R)-3-hydroxy-5-(2-isobutyr-amido-6-oxo-1,6-dihydro-9H-purin-9-yl)-4-methoxytetrahydrofuran-2-yl)methyl)stearamide

To a stirred solution of N-(2-((2S,3R,4R,5R)-2-((bis(4-methoxyphenyl)(phenyl) methoxy)methyl)-5-(2,4-dioxo-3,4-dihydro pyrimidin-1(2H)-yl)-4-methoxytetrahydrofuran-3-yl)ethyl)-N-(((2R,3R,4R,5R)-3-((tert-butyl-dimethylsilyl) oxy)-5-(2-isobutyramido-6-oxo-1,6-dihydro-9H-purin-9-yl)-4-methoxytetrahydro-furan-2-yl) methyl)stearamide (3.0 g, 2.3 mmol) in DMSO (30 mL) were added CsF (0.69 g, 4.55 mmol) in portions at RT. The resulting mixture was stirred for 2 hours at RT under air atmosphere. The reaction was quenched by the addition of saturated aqueous NaHCO₃ (10 mL) at RT. The aqueous layer was extracted with EA (3×100 mL). The combined EA layer was washed with brine (3×50 mL), dried by Na₂SO₄, filtered and concentrated. The residue was purified by silica gel column chromatography, eluted with MeOH:DCM (1% TEA) (1:10) to afford the titled compound (2.0 g, 69% yield) as an off-white solid. MS(ESI, negative mode): m/z=1201.8[M−H]⁻.

### Step 5: (2R,3R,4R,5R)-2-((N-(2-((2S,3R,4R,5R)-2-((bis(4-methoxyphenyl)(phenyl)methoxy) methyl)-5-(2,4-dioxo-3,4-dihydropyrimidin-1(2H)-yl)-4-methoxytetrahydrofuran-3-yl)ethyl)stearamido)methyl)-5-(2-isobutyramido-6-oxo-1,6-dihydro-9H-purin-9-yl)-4-methoxytetrahydrofuran-3-yl(2-cyanoethyl) diisopropylphosphoramidite

To a stirred suspension of 4 A MS in DCM (40 mL) were added 4,5-dicyanoimidazole (0.29 g, 2.49 mmol) and 2-cyanoethyl N,N,N′,N′-tetraisopropylphosphorodiamidite (0.75 g, 2.49 mmol) at RT. The resulting mixture was stirred for 10 minutes at RT under argon atmosphere. To the above mixture was added N-(2-((2S,3R,4R,5R)-2-((bis(4-methoxyphenyl)(phenyl)methoxy)methyl)-5-(2,4-dioxo-3,4-dihydro pyrimidin-1(2H)-yl)-4-methoxytetrahydrofuran-3-yl)ethyl)-N-(((2R,3R,4R,5R)-3-hydroxy-5-(2-isobutyramido-6-oxo-1,6-dihydro-9H-purin-9-yl)-4-methoxytetrahydrofuran-2-yl)methyl)stearamide (2.0 g, 1.7 mmol) in DCM (20 mL) dropwise at RT. The resulting mixture was stirred for additional 1 hour at RT under argon atmosphere. The reaction mixture was filtered and the filter cake was washed with DCM (contained 0.5% TEA, 3×10 mL). The reaction was quenched by the addition of saturated aqueous NaHCO₃ solution (10 mL) at 0° C. The mixture was extracted with DCM (contained 0.5% TEA, 3×50 mL). Combined the organic layers and washed with water (2×25 mL). The organic layer was dried by MgSO₄, filtered and concentrated. The residue was purified by silica gel column chromatography, eluted with MeOH:DCM (1% TEA) (1:10) to afford the titled compound NB-206 (1.4 g, 0.947 mmol, 57.08%) as an off-white solid. ³¹P NMR (162 MHz, DMSO-d₆) δ 149.97, 149.68, 149.61, 149.17. MS (ESI, negative mode): m/z=1402.2[M−H]⁻. Oligonucleotide comprising dinucleotide NB-206 has been synthesized using the general procedure described in example 1.

### Example 96: Synthesis of NB-104

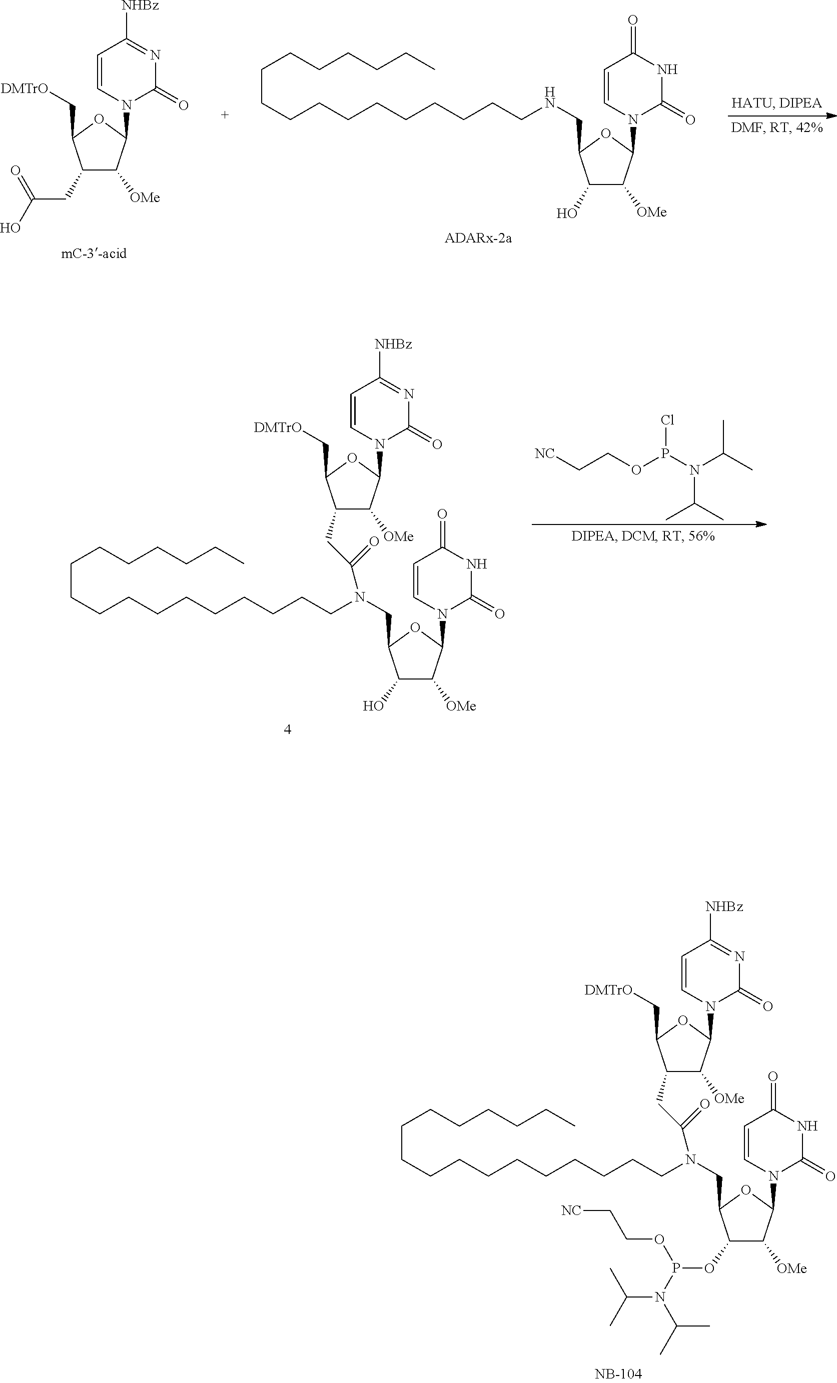

A solution of mC-3′-acid (342 mg, 0.485 mmol, 1 eq), DIPEA (0.211 mL, 1.213 mmol, 2.5 eq) and HATU (221 mg, 0.582 mmol, 1.1 eq) in DMF (10 mL) was stirred for 15 min at room temperature, then amine ADARx-2a (264 mg, 0.534 mmol, 1.2 eq) was added and the mixture was stirred for 48 h at room temperature, reaction mixture was diluted with water (20 ml), extracted with Ethyl acetate 2×100 mL, combined organics were washed with Aq. Sat. NaHCO₃, and brine solution, dried over Na₂SO₄, and concentrated, the crude residue was purified by Ethyl acetate/Hex, 0-100% as an eluent, pure fractions were combined and concentrated to obtain amide 4 (240 mg, 42%) as a brown solid. NMR and LCMS m/z 1205 (M+Na) are corresponding with the product.

To a stirred solution of alcohol 4 (230 mg, 0.194 mmol, 1 eq) and diisopropylethylamine (0.2 mL, 1.167 mmol, 6 eq), in DCM (10 mL), was added N, N-diisopropyl chlorophosphoramidite (0.13 mL 0.583 mmol, 3 eq) dropwise. The reaction mixture was stirred at room temperature for 12 h. LCMS showed starting material. Another 1.5 eq of reagent (0.065 mL 0.291 mmol, 1.5 eq) was added and the mixture was stirred for another 12 h. Reaction mixture was quenched with Aq. Saturated NaHCO₃ solution (20 mL), extracted with DCM (100) ml, washed with brine (100 mL), dried over N2SO₄, and evaporated the crude product was loaded on to (pre-equilibrated with 1% Et₃N-EtOAc) Biotage silica gel column (25 g 20 μm), and purified by flash chromatography using 0-10% MeOH/EtOAc containing 1% Et₃N as an additive. Pure fractions were combined and concentrated, dried under high vacuum to obtain Phosphoramidite NB-104 (150 mg, 56%) as a white solid. 95% purity by HPLC, P31-NMR, Mass (m/z 1405 M+Na), ³¹P NMR (202 MHz, DMSO-d₆) δ 149.72, 149.53, 149.58.

### Example 97: Synthesis of NB-114

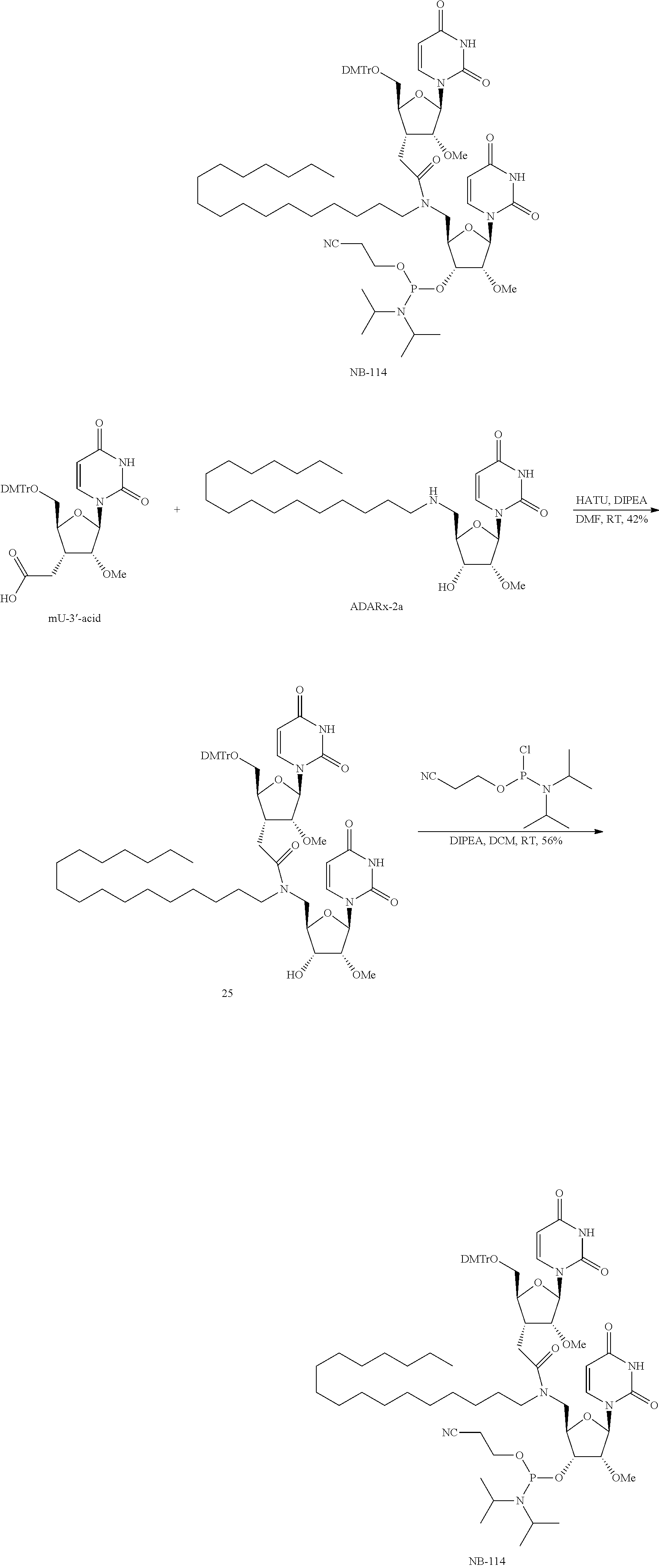

A solution of acid mU-3′-acid (0.596 g, 0.990 mmol, 1 eq), DIPEA (0.516 mL, 2.97 mmol, 3 eq) and HATU (0.564 g, 1.485 mmol, 1.5 eq) in DMF (10 mL) was stirred for 15 min at room temperature, then amine ADARx-2a (0.539 g, 1.089 mmol, 1.1 eq) was added and the mixture was stirred for 3 h at room temperature, reaction mixture was added dropwise in to water (200 ml), precipitated solids were filtered and washed with water, the solids were dried and re dissolved in DCM then purified by MeOH/Ethyl acetate, 0-10% as an eluent, pure fractions were combined and concentrated to obtain amide 25 (1.65 g, 86%) as a brown solid. NMR and LCMS m/z 1081 (M+1) are corresponding with the product.

To a stirred solution of alcohol 25 (0.835 g, 0.773 mmol, 1 eq) and diisopropylethylamine (0.8 mL, 4.639 mmol, 6 eq), in DCM (10 mL), was added N, N-diisopropyl chlorophosphoramidite (0.5 mL 2.3 mmol, 3 eq) dropwise. The reaction mixture was stirred at room temperature for 1 h. Reaction mixture was quenched with Aq. Saturated NaHCO₃ solution (30 mL), extracted with DCM (50) ml, washed with brine (50 mL), dried over N2SO₄, and evaporated the crude product was loaded on to (pre-equilibrated two times with 1% Et₃N-DCM) Biotage silica gel column (25 g 20 μm), and purified by flash chromatography using 0-10% MeOH/DCM containing 1% Et₃N as an additive. Pure fractions were combined and concentrated, dried under high vacuum to obtain Phosphoramidite NB-114 (750 mg, 75%) product as a white solid. 95% purity by HPLC, Mass (m/z 1302 M+Na), ³¹P NMR (202 MHz, DMSO-d₆) δ 149.81, 149.69, 149.47, 149.34.

### Example 98: Synthesis of NB-115

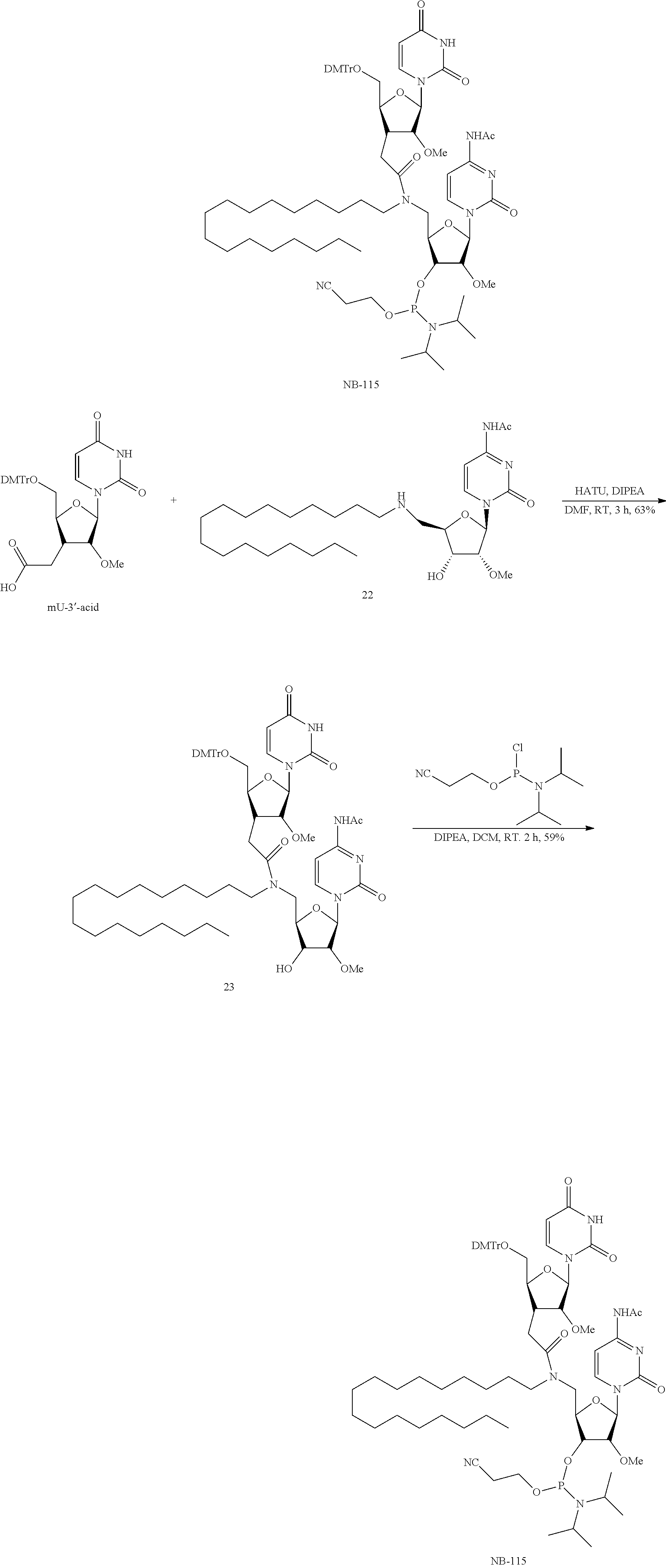

A solution of acid 21 (0.596 g, 0.990 mmol, 1 eq), DIPEA (0.516 mL, 2.97 mmol, 3 eq) and HATU (0.564 g, 1.485 mmol, 1.5 eq) in DMF (10 mL) was stirred for 15 min at room temperature, then amine 22 (0.584 g, 1.089 mmol, 1.1 eq) was added and the mixture was stirred for 3 h at room temperature, reaction mixture was added dropwise in to water (200 ml), precipitated solids were filtered and washed with water, the solids were dried and re dissolved in DCM then purified by MeOH/EtOAc. 0-10% as an eluent, pure fractions were combined and concentrated to obtain amide 23 (1.1 g, 63%) as a brown solid. NMR and LCMS m/z 1122 (M+1) are corresponding with the product.

To a stirred solution of alcohol 23 (0.570 g, 0.508 mmol, 1 eq) and diisopropylethylamine (0.53 mL, 3.05 mmol, 6 eq), in DCM (10 mL), was added N, N-diisopropyl chlorophosphoramidite (0.34 mL 1.52 mmol, 3 eq) dropwise. The reaction mixture was stirred at room temperature for 2 h. Reaction mixture was quenched with Aq. Saturated NaHCO₃ solution (30 mL), extracted with DCM (50) ml, washed with brine (50 mL), dried over N2SO₄, and evaporated the crude product was loaded on to (pre-equilibrated two times with 1% Et₃N-DCM) Biotage silica gel column (25 g 20 μm), and purified by flash chromatography using 0-10% MeOH/DCM containing 1% Et₃N as an additive. Pure fractions were combined and concentrated, dried under high vacuum to obtain Phosphoramidite NB-115 (400 mg, 59%) product as a white solid. 96% purity by HPLC, Mass (m/z 1344 M+Na), ³¹P NMR (202 MHz, DMSO-d₆) δ 149.94, 149.66, 149.24.

### Example 99: Synthesis of compound NB-116

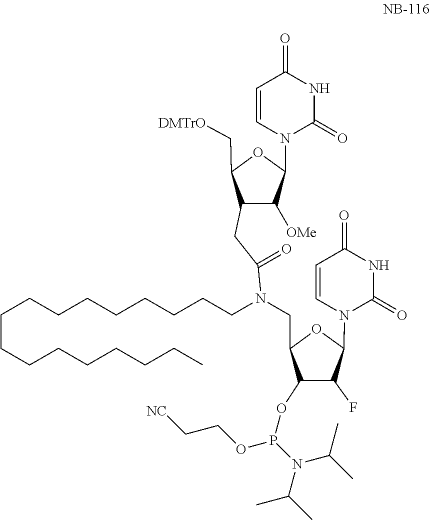

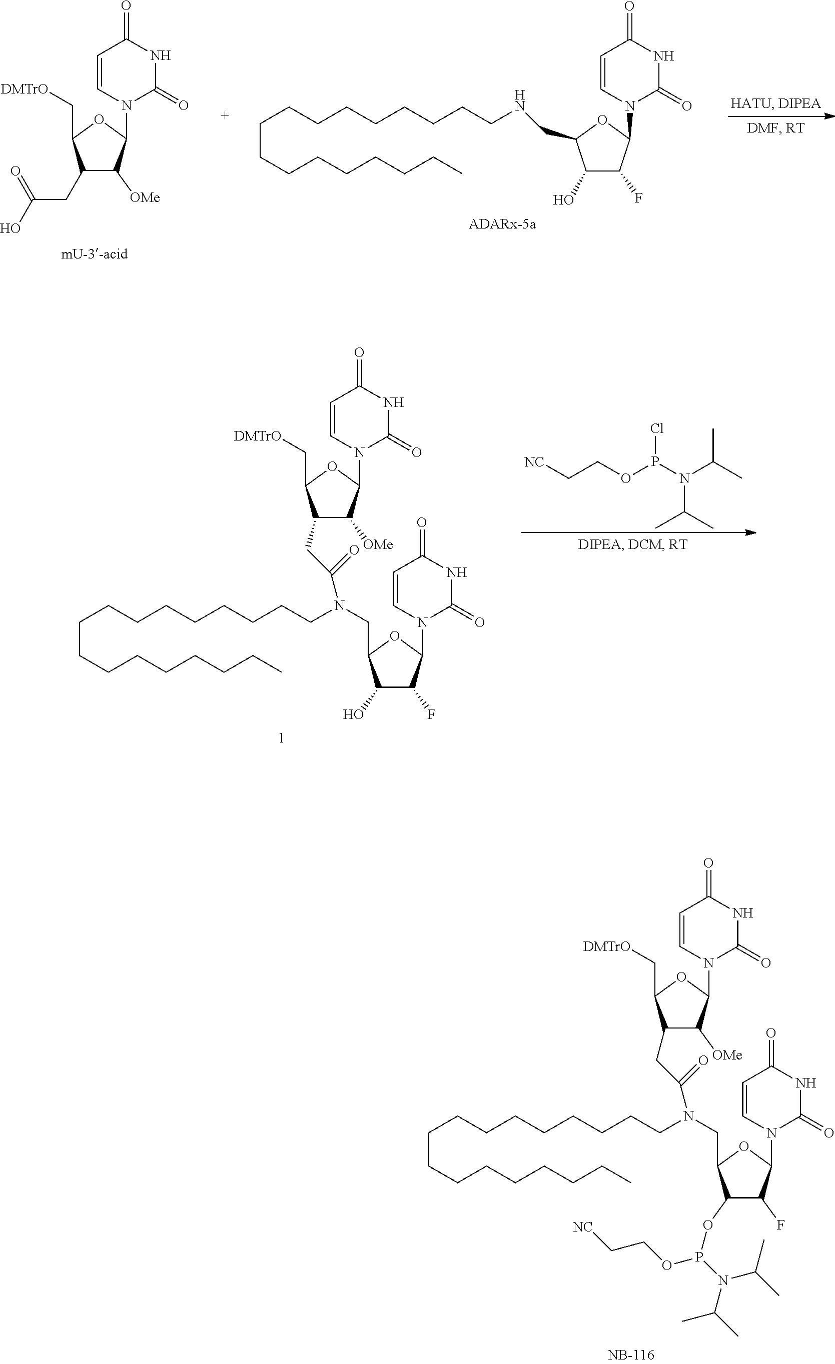

A solution of mU-3′-acid (0.900 g, 1.495 mmol, 1 eq), DIPEA (0.78 mL, 4.485 mmol, 3 eq) and HATU (0.852 g, 2.243 mmol, 1.5 eq) in DMF (10 mL) was stirred for 15 min at room temperature, then amine ADARx-5a (0.794 g, 1.645 mmol, 1.1 eq) was added and the mixture was stirred for 3 h at room temperature, reaction mixture was diluted with water (50 ml), precipitated solids were filtered and washed with water, the solids were dried and re dissolved in DCM then purified by Ethyl acetate/MeOH, 0-5% as an eluent, pure fractions were combined and concentrated to obtain amide 1 (1.025 g, 64%) as a brown solid. LCMS m/z 1091 (M+Na).

To a stirred solution of alcohol 1 (0.775 g, 0.726 mmol, 1 eq) and diisopropylethylamine (0.757 mL, 4.354 mmol, 6 eq), in DCM (10 mL), was added N, N-diisopropyl chlorophosphoramidite (0.484 mL 2.177 mmol, 3 eq) dropwise. The reaction mixture was stirred at room temperature for 2 h. Reaction mixture was quenched with Aq. Saturated NaHCO₃ solution (30 mL), extracted with DCM (50) ml, washed with brine (50 mL), dried over N2SO₄, and evaporated the crude product was loaded on to (pre-equilibrated two times with 1% Et₃N-DCM) Biotage silica gel column (25 g 20 μm), and purified by flash chromatography using 0-10% MeOH/DCM containing 1% Et₃N as an additive. Pure fractions were combined and concentrated, dried under high vacuum to obtain Phosphoramidite NB-116 (650 mg, 70%) product as a white solid. 96% purity by HPLC, Mass (m/z 1290 M+Na), ³¹P NMR (202 MHz, DMSO-d₆) δ 150.14, 150.04, 149.98, 149.76.

### Example 100: Synthesis of compound NB-117

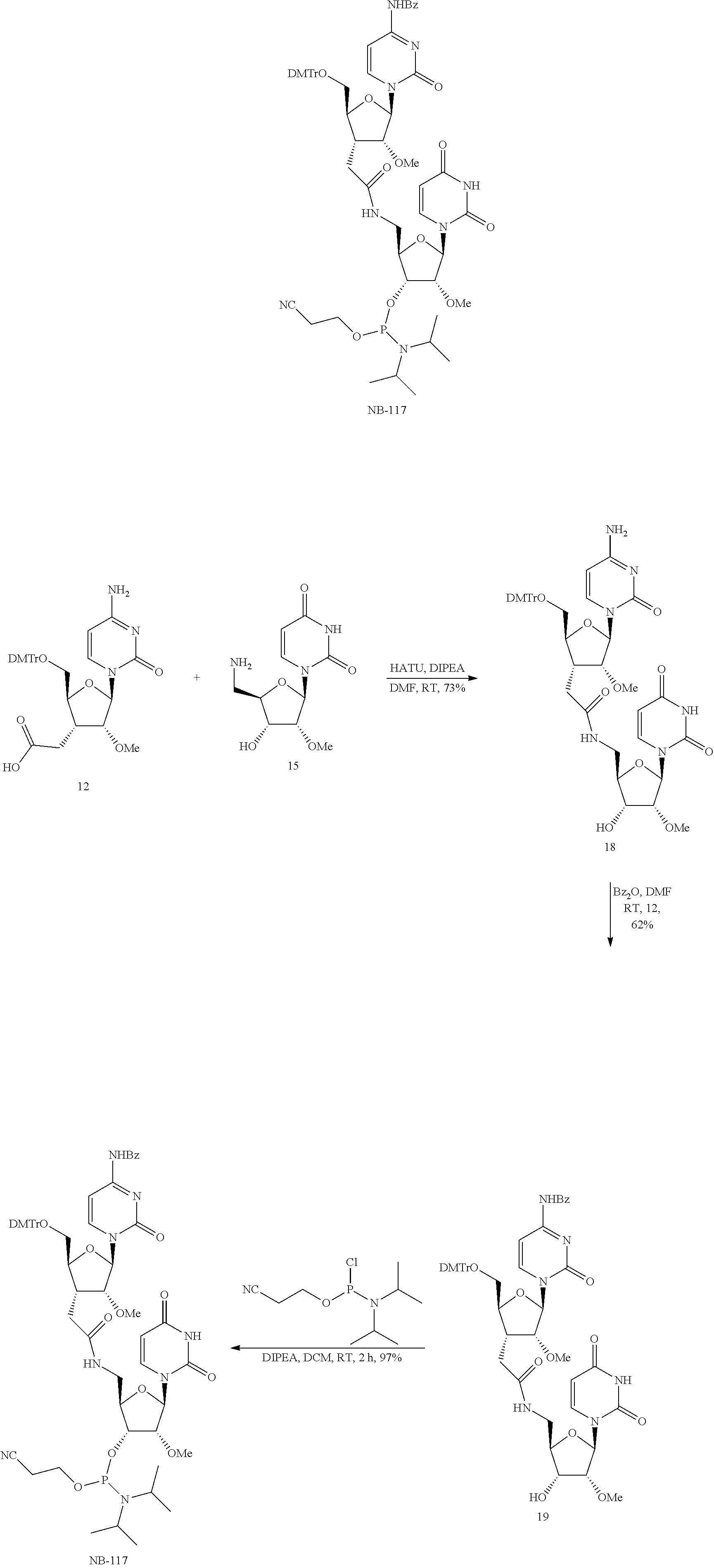

A solution of acid 12 (1 g, 1.664 mmol, 1 eq), DIPEA (0.868 mL, 4.992 mmol, 3 eq) and HATU (0.948 g, 2.496 mmol, 1.5 eq) in DMF (10 mL) was stirred for 15 min at room temperature, then amine 15 (0.450 g, 1.75 mmol, 1.05 eq) was added and the mixture was stirred for 3 h at room temperature, reaction mixture was diluted with water (50 ml), precipitated solids were filtered and washed with water, the solids were dried and re dissolved in DCM then purified by Ethyl acetate/MeOH, 0-5% as an eluent, pure fractions were combined and concentrated to obtain amide 18 (1.024 g, 73%) as a brown solid. NMR and LCMS m/z 841 (M+1) are corresponding with the product.

A mixture of amine 18 (0.918 g, 1.093 mmol, 1 eq) and Benzoic anhydride (296 mg, 1.311 mmol, 1.2 eq) was in DMF (10 mL) was stirred overnight at RT. The solution was diluted with EtOAc 100 mL, the organic phase washed with aq NaCl solution 50 mL, organic phase was dried over Na₂SO₄, and concentrated in vacuo. The residue was purified by flash chromatography on silica gel. using MeOH/EtOAc. 0-10% as an eluent. Pure fractions were combined and concentrated to obtain compound 19 (640 mg, 62%) as a white solid. NMR and LCMS are corresponding with product.

To a stirred solution of alcohol 19 (0.540 g, 0.571 mmol, 1 eq) and diisopropylethylamine (0.596 mL, 3.429 mmol, 6 eq), in DCM (10 mL), was added N, N-diisopropyl chlorophosphoramidite (0.381 mL 1.714 mmol, 3 eq) dropwise. The reaction mixture was stirred at room temperature for 2 h. Reaction mixture was quenched with Aq. Saturated NaHCO₃ solution (30 mL), extracted with DCM (50) ml, washed with brine (50 mL), dried over N2SO₄, and evaporated the crude product was loaded on to (pre-equilibrated two times with 1% Et₃N-DCM) Biotage silica gel column (25 g 20 μm), and purified by flash chromatography using 0-10% MeOH/DCM containing 1% Et₃N as an additive. Pure fractions were combined and concentrated, dried under high vacuum to obtain Phosphoramidite NB-117 (640 mg, 97%) product as a white solid. 96% purity by HPLC, Mass (m/z 1167 M+Na), H1-NMR are corresponding with product. ³¹P NMR (202 MHz, DMSO-d₆) δ 150.03, 149.65.

### Example 101: Synthesis of NB-211

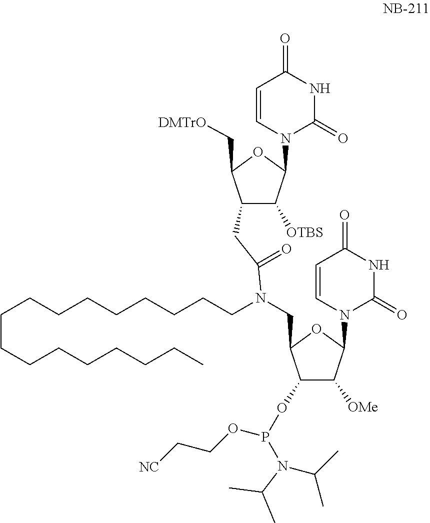

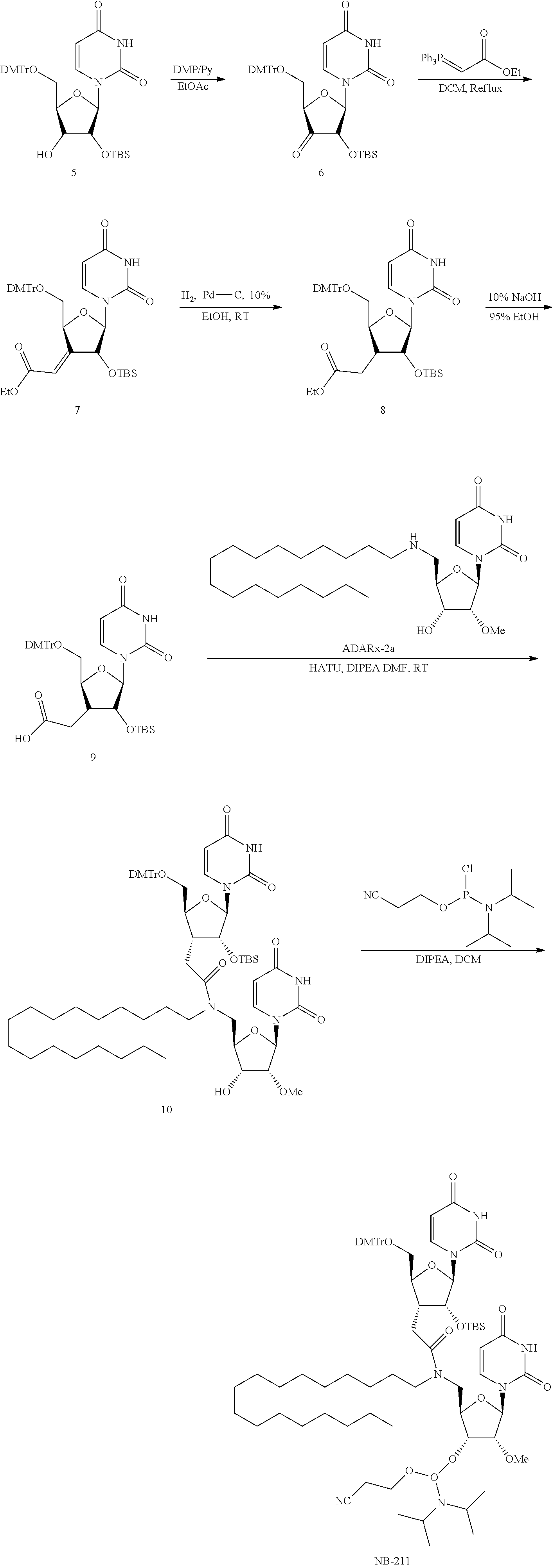

### Synthesis of acid 9

To a stirred mixture of DMP (9.64 g, 22.727 mmol, 1.5 eq.) in EtOAc (200 mL) was added pyridine (3.6 mL 45.46 mmol 3 eq) at 0° C., the suspension was stirred for 30 min then alcohol 5 (10.00 g, 15.152 mmol, 1 eq.) was added portion wise at 0° C. under Argon atmosphere. The mixture was stirred at room temperature for 12 h. Reaction mixture was filtered, washed with EtOAc (100 mL). Filtrate was concentrated and the resulting solids were diluted with EtOAc (300 mL), washed with aq saturated NaHCO₃ solution (2×100 mL), dried over Na₂SO₄, concentrated to dryness. The crude product was purified by column chromatography using 0-100% EtOAc/Hexane as an eluent to obtain ketone 6, 9.027 g, 90.5% as a white solid. LCMS m/z 659 (M+1).

A mixture of ketone 6 (4.73 g, 7.188 mmol, 1 eq) and (ethoxycarbonylmethylene) triphenylphosphorane (3.252 g, 9.345 mmol, 1.3 eq), in dry CH₂Cl₂ (50 mL) was heated to reflux for 6 h. After cooling down to room temperature, the resulting mixture was concentrated and purified by silica gel column (EtOAc/hexane 0-50%) to afford the olefin 7 (4.87 g 74%) as a white solid. LCMS (m/z 751 M+Na).

Olefin 7 (2.5 g, 3.434 mmol) was dissolved Ethanol (40 mL) and the reaction mixture was degassed by purging with nitrogen. Then 10% Pd/C (250 mg, 10% w/w) was added to the reaction mixture, purged with hydrogen gas from a balloon and stirred under slight positive pressure of hydrogen (balloon) at room temperature for 4 days. LCMS showed complete reduction. Reaction mixture was filtered through celite and concentrated. The residue was purified by column chromatography using 0-50% Ethyl acetate/Hexane as an eluent, pure fractions were combined and concentrated to obtain desired product 8 (1.93 g 77%) as a white solid. LCMS (m/z=753 M+Na).

A 10% aqueous NaOH solution (0.75 mL) was added to a solution of ester 8 (600 mg, 0.52 mmol) in 95% ethanol (10 mL) and the resulting mixture stirred for 12 h at room temp. LCMS showed complete hydrolysis, Ethanol was evaporated the residue was diluted with water 5 mL, acidified by the careful addition of a 10% aqueous HCl solution until the pH 6.5. The solution was diluted with CH₂Cl₂ 100 mL, the organic phase washed with brine and concentrated in vacuo. The residue was purified by flash chromatography on silica gel. using hexane/EtOAc. 0-100% as an eluent. Pure fractions were combined and concentrated to obtain acid 9 (405 mg, 70%) as a white solid. LCMS m/z 703 (M+1).

### Synthesis of NB-211

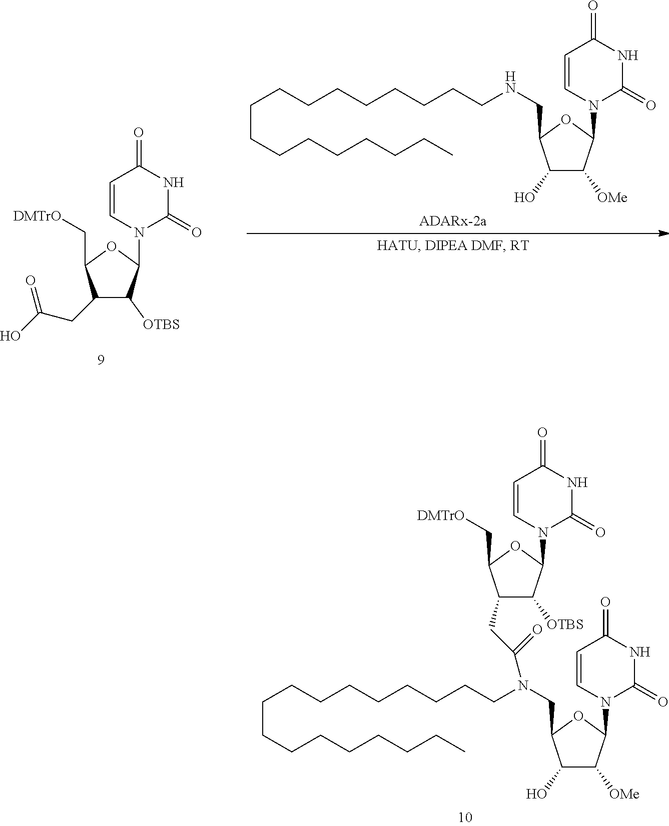

2-((2S,3R,4R,5R)-2-((bis(4-methoxyphenyl)(phenyl)methoxy)methyl)-4-((tert-butyldimethylsilyl)oxy)-5-(2,4-dioxo-3,4-dihydropyrimidin-1(2H)-yl)tetrahydrofuran-3-yl)-N-(((2R,3R,4R,5R)-5-(2,4-dioxo-3,4-dihydropyrimidin-1(2H)-yl)-3-hydroxy-4-methoxytetrahydrofuran-2-yl)methyl)-N-heptadecylacetamide.

To a solution of acid 9 (1.2 g, 1.7 mmol, 1 eq) and amine ADARx-2a (1.1 g, 2.2 mmol, 1.3 eq), in DMF (10 mL), was added HATU (0.99 g, 2.6 mmol, 1.5 eq) and DIPEA (1 mL, 5.1 mmol, 3 eq), the mixture was stirred for 30 min. at RT. Reaction mixture was diluted with water, extracted with DCM, dried over Na₂SO₄, filtered and concentrated, purified by Biotage (50 g, 20 micron column) 0 to 10% DCM:MeOH 8 CV, 10 to 10% 5 cv yielded desired product 10 (1.2 g, 55%) as white solid (LCMS shows M+H).

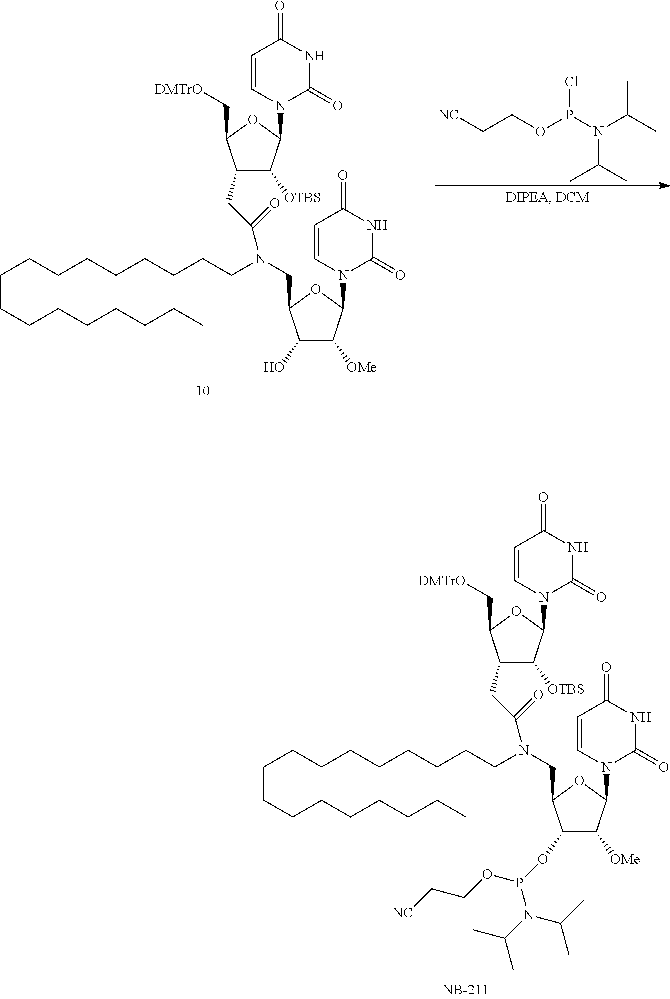

(2R,3R,4R,5R)-2-((2-((2S,3R,4R,5R)-2-((bis(4-methoxyphenyl)(phenyl) methoxy)methyl)-4-((tert-butyldimethylsilyl)oxy)-5-(2,4-dioxo-3,4-dihydropyrimidin-1(2H)-yl)tetrahydrofuran-3-yl)-N-heptadecylacetamido)methyl)-5-(2,4-dioxo-3,4-dihydropyrimidin-1(2H)-yl)-4-methoxytetrahydrofuran-3-yl (2-cyanoethyl) diisopropylphosphoramidite-NB-211:

To a stirred solution of alcohol 10 (1.1 g, 0.931 mmol, 1 eq) and DIPEA (1 mL, 5.59 mmol, 6 eq), in DCM (10 mL), was added N, N-diisopropyl chlorophosphoramidite (0.65 mL, 2.79 mmol, 3 eq) dropwise. The reaction mixture was stirred at room temperature for 1 hr. Quenched with aqueous saturated NaHCO₃, extracted with DCM, dried over Na₂SO₄ and concentrated, purified by flash chromatography using MeOH/EtOAc 0-1% 10 CV, then 1 to 100% 2 CV, containing 1% Et₃N as an additive, gave NB-211 (461 mg 36%) as a white solid. ³¹P NMR (202, MHz, DMSO-d6) δ 149.91, 149.67, 149.40, 148.88. Mass (ESI) m/z: [M+Na]⁺ Cacld for 1403.1 found: 1403.3.

### Example 102: Effect of RD2547 targeting mouse CTNNB1 (Catenin Beta 1)

The compound RD2547 has the following structure:

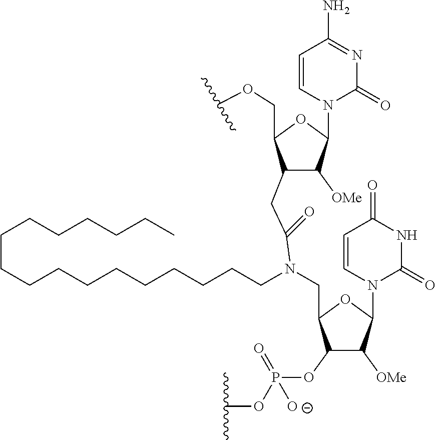

attached at the 3^{rd} and 4^{th} nucleoside from the 5′ end of the sense strand. RD2547 was evaluated in an in vivo mouse PD study.

TABLE 1
Chemical Nomenclature.

| Abbreviation | Structure |
|---|---|
| ‘e’ | 5′-ethylenephosphonate modification |
| ‘v’ | 5′-vinylphosphonate modification |
| ‘m’ | 2′-O-methyl sugar modification (e.g., mA, mG, mC, mU, mX) |
| ‘f’ | 2′-F sugar modification (e.g., fA, fG, fC, fU, fX) |
| ‘*’ | Phosphorothioate internucleoside linkage |
| ‘.’ | Phosphate internucleoside linkage |
| ‘X’ | Any base (A, G, C, U) |
| ‘dQ’ | Inverted abasic deoxyribose |
| ‘B7’ | C18 lipid |
| ‘ë’ | Backbone: Amide internucleoside linkage with lipid |
| ‘ê’ | Backbone: Amide internucleoside linkage |
| ‘\ddot{1}’ | Backbone: Carbamate internucleoside linkage with lipid |
| ‘â’ | Backbone: Carbamate internucleoside linkage |
| ‘ö’ | Backbone: Amide internucleoside linkage with lipid |
| | functionalized 1,2,3-triazole |
| ‘[ë1]’ | Backbone: Amide internucleoside linkage with an isopropyl group |
| ‘[ë2]’ | Backbone: Amide internucleoside linkage with a pentyl group |
| ‘AC6’ | 5′-Amino-Modifier C6-TFA (i.e., 6-(Trifluoroacetylamino)- |
| | hexyl-(2-cyanoethyl)-(N,N-diisopropyl)-phosphoramidite) |
| ‘SC6’ | 3′-Thiol-Modifier C6 S—S (i.e., 1-O-Dimethoxytrityl-hexyl-disulfide,1′- |
| | [(2-cyanoethyl)-(N,N-diisopropyl)]-phosphoramidite) |
| ‘CB’ or ‘CBM’ | Cerebellum |
| ‘TCtx’ | Temporal cortex |
| ‘CTX’ | Frontal cortex |
| ‘HPC’ | Hippocampus |
| ‘STR’ | Striatum |
| ‘BS’ | Brainstem |
| ‘CER’ or ‘CERV’ | Cervical spinal cord |
| ‘THOR’ | Thoracic spinal cord |
| ‘LU’ or ‘LUMB’ | Lumbar spinal cord |
| ‘SC’ | Spinal cord |
| ‘D 8’ | Day 8 |
| ‘D 15’ | Day 15 |
| ‘D 29’ | Day 29 |
| ‘D 43’ | Day 43 |
| ‘D 57’ | Day 57 |
| ‘D 73’ | Day 73 |
| ‘D 83’ | Day 83 |
| ‘D 98’ | Day 98 |
| ‘D 126’ | Day 126 |
| ‘D 168’ | Day 168 |

TABLE 2
Parent oligonucleotide used for the Examples below.

| | | Ref ID |
|---|---|---|
| Compound | Modified Strands (5′-3′) | NO: |
| RD2540 | (vU)*(fU)*(mU)(mC)(mG)(fA)(mA)(fU)(fC)(mA)(mA)(mU) | IA0883 |
| | (mC)(fC)(mA)(fA)(mC)(mA)(mG)(mU)(mA)*(mG)*(mC) | |
| | (mU)*(mA)*(mC)(mU)(mG)(mU)(fU)(mG)(fG)(fA)(fU)(mU) | IS1096 |
| | (mG)(mA)(mU)(mU)(mC)(mG)(mA)(mA)*(mA)*(dQ) | |

Six animals received a single 72 μg dose via intrathecal injection on day 1. Animals were observed every day for behavioral changes. Frontal Cortex was collected from half the animals on day 22 and the other half at day 36. Tissue was immediately placed in homogenizing tube, snap frozen, then be kept in the −80° C. for gene expression analysis. The remainder of the cortex was collected for PK analysis.

RNA Isolation was performed according to the NucleoSpin 96 RNA Core kit (Macherey-Ngael Cat #740466.4) instructions. Following RNA isolation, a 96-well plate was placed on ice while the qRT-PCR reaction was prepared. 2 μl of RNA was added to the reaction mixture containing 5 μl TaqMan Fast Virus 1-Step Master Mix (Thermo Fisher #44444432), 1 μl CTNNB1 TaqMan Gene Expression Assay (Thermo Fisher: Rn00584431_g1, FAM), 1 μl ACTB (VIC) TaqMan Gene Expression Assay (Thermo Fisher: Rn00667869_m1, VIC) and 11 μl RT-PCR grade nuclease-free water in a MicroAmp Optical 96-well plate (0.2 mL). qPCR was performed using a QuantStudio3 qPCR machine with the following cycles: 50° C. for 1 minute, 95° C. for 20 seconds and 40 cycles at 95° C. for 15 seconds and 60° C. for 1 minute. Results are presented in the tables below as percent inhibition of CTNNB1, relative to vehicle control.

TABLE 3

| | | | Ref ID |
|---|---|---|---|
| Compound | Backbone | Modified Strands (5′-3′) | NO: |
| RD2547 | mCëmU | (vU)*(fU)*(mU)(mC)(mG)(fA)(mA)(fU)(fC)(mA) | IA0883 |
| | NB-104 | (mA)(mU)(mC)(fC)(mA)(fA)(mC)(mA)(mG)(mU) | |
| | | (mA)*(mG)*(mC) | |
| | | (mU)*(mA)*(mCëmU)(mG) | IS1103 |
| | | (mU)(fU)(mG)(fG)(fA)(fU)(mU)(mG)(mA)(mU) | |
| | | (mU)(mC)(mG)(mA)(mA)*(mA)*(dQ) | |

TABLE 4
Average CTNNB1 Inhibition.

Days

| Compound | 22 | 36 |
|---|---|---|
| RD2547 | 15 | 62 |

### Example 103: Effect of RD2540, RD2547 and RD2548 Targeting Rat CTNNB1

The compounds RD2540 and RD2547 are described above. The compound RD2548 has the following structure:

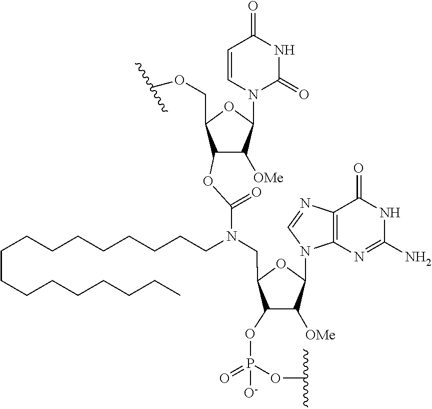

attached at the 4^{th} and 5^{th} nucleoside from the 5′ end on the sense strand and is further described below in Table 5. RD2540, RD2547 and RD2548 were evaluated in an in vivo rat PD study. Six animals received a single 0.9 mg (3 mg/kg) dose via intrathecal injection on day 1. Animals were observed every day for behavioral changes. Frontal Cortex was collected from half the animals on day 15 and the other half at day 29. Tissue was immediately placed in homogenizing tube, snap frozen, then be kept in the −80° C. for gene expression analysis. The remainder of the cortex was collected for PK analysis.

RNA Isolation was performed according to the RNeasy Micro Kit (Qiagen Cat #74004) instructions. Following RNA isolation, a 96-well plate was placed on ice while the qRT-PCR reaction was prepared. 2 μl of RNA was added to the reaction mixture containing 5 μl TaqMan Fast Virus 1-Step Master Mix (Thermo Fisher #44444432), 1 μl CTNNB1 TaqMan Gene Expression Assay (Thermo Fisher: Rn00584431_g1, FAM), 1 μl ACTB (VIC) TaqMan Gene Expression Assay (Thermo Fisher: Rn00667869_ml, VIC) and 11 μl RT-PCR grade nuclease-free water in a MicroAmp Optical 96-well plate (0.2 mL). qPCR was performed using a QuantStudio3 qPCR machine with the following cycles: 50° C. for 1 minute, 95° C. for 20 seconds and 40 cycles at 95° C. for 15 seconds and 60° C. for 1 minute. Results are presented in the table below as percent inhibition of CTNNB1, relative to vehicle control.

TABLE 5

| | | | Ref ID |
|---|---|---|---|
| Compound | Backbone | Modified Strands (5′-3′) | NO: |
| RD2548 | mUïmG | (vU)*(fU)*(mU)(mC)(mG)(fA)(mA)(fU)(fC)(mA)(mA) | IA0883 |
| | NB-106 | (mU)(mC)(fC)(mA)(fA)(mC)(mA)(mG)(mU)(mA)*(mG)* | |
| | | (mC) | |
| | | (mU)*(mA)*(mC)(mUïmG) | IS1104 |
| | | (mU)(fU)(mG)(fG)(fA)(fU)(mU)(mG)(mA)(mU)(mU) | |
| | | (mC)(mG)(mA)(mA)*(mA)*(dQ) | |

TABLE 6
Average CTNNB1 Inhibition

Days

| Compound | 15 | 29 |
|---|---|---|
| RD2540 | 31 | 13 |
| RD2547 | 54 | 21 |
| RD2548 | 13 | 4 |

### Example 104: Effect of RD2855, RD2856 and RD2857 Targeting Rat CTNNB1

The compound RD2855 has the following structure:

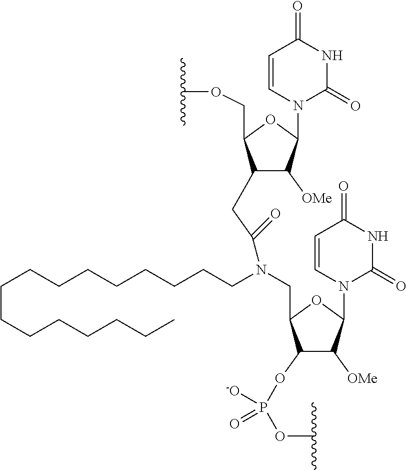

attached at the 15^{th} and 16^{th} nucleoside from the 5′end on the sense strand. The compound RD2856 has the following structure:

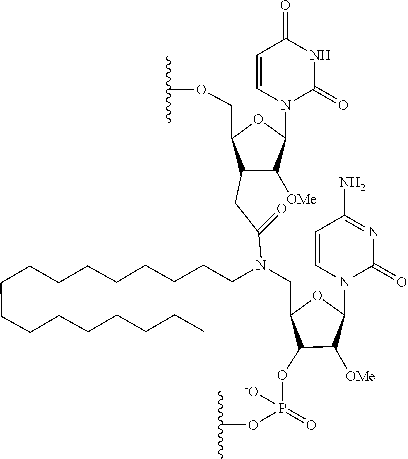

attached at the 16^{th} and 17^{th} nucleoside from the 5′ end on the sense strand. The compound RD2857 has the following structure:

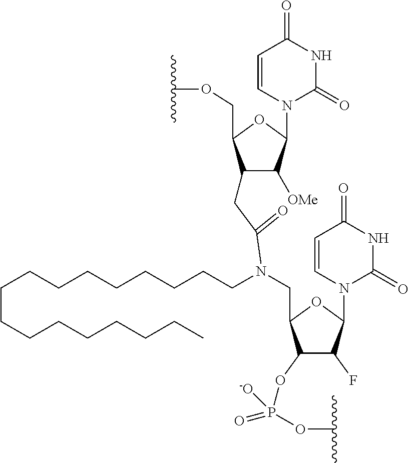

attached at the 6^{th} and 7^{th} nucleoside from the 5′end on the sense strand. RD2855, RD2856 and RD2857 were evaluated in an in vivo rat PD study as described in Example 103 above. RNA Isolation and qPCR was performed as described in Example 103 above. Results are presented in the table below as percent inhibition of CTNNB1, relative to vehicle control.

TABLE 7

| | | | Ref ID |
|---|---|---|---|
| Compound | Backbone | Modified Strands (5′-3′) | NO: |
| RD2855 | mUëmU | (vU)*(fU)*(mU)(mC)(mG)(fA)(mA)(fU)(fC)(mA) | IA0883 |
| | NB-114 | (mA)(mU)(mC)(fC)(mA)(fA)(mC)(mA)(mG)(mU) | |
| | | (mA)*(mG)*(mC) | |
| | | (mU)*(mA)*(mC)(mU)(mG)(mU)(fU)(mG)(fG) | IS1265 |
| | | (fA)(fU)(mU)(mG)(mA)(mUëmU)(mC)(mG)(mA) | |
| | | (mA)*(mA)*(dQ) | |
| RD2856 | mUëmC | (vU)*(fU)*(mU)(mC)(mG)(fA)(mA)(fU)(fC)(mA) | IA0883 |
| | NB-115 | (mA)(mU)(mC)(fC)(mA)(fA)(mC)(mA)(mG)(mU) | |
| | | (mA)*(mG)*(mC) | |
| | | (mU)*(mA)*(mC)(mU)(mG)(mU)(fU)(mG)(fG) | IS1266 |
| | | (fA)(fU)(mU)(mG)(mA)(mU)(mUëmC)(mG)(mA) | |
| | | (mA)*(mA)*(dQ) | |
| RD2857 | mUëfU | (vU)*(fU)*(mU)(mC)(mG)(fA)(mA)(fU)(fC)(mA) | IA0883 |
| | NB-116 | (mA)(mU)(mC)(fC)(mA)(fA)(mC)(mA)(mG)(mU) | |
| | | (mA)*(mG)*(mC) | |
| | | (mU)*(mA)*(mC)(mU)(mG)(mUëfU)(mG)(fG)(fA) | IS1267 |
| | | (fU)(mU)(mG)(mA)(mU)(mU)(mC)(mG)(mA)(mA) | |
| | | *(mA)*(dQ) | |

TABLE 8
Average CTNNB1 Inhibition.

Days

| Compound | 15 | 29 |
|---|---|---|
| RD2855 | 60 | 31 |
| RD2856 | 61 | 52 |
| RD2857 | 48 | 57 |

### Example 105: Effect of RD2861, RD2854, RD2894, RD2895, RD2896 and RD2897 Targeting Rat CTNNB1

The compound RD2861 has a C18 lipid conjugate attached to the 5′ end of the sense strand and has the following structure:

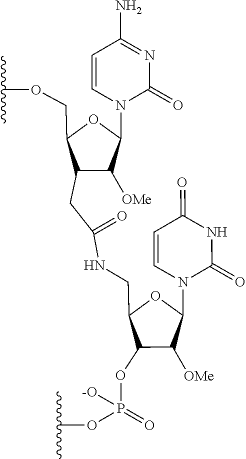

attached at the third and fourth nucleoside from the 5′ end of the sense strand. The compound RD2854 has a C18 lipid conjugate attached to the 5′ end of the sense strand and has the following structure:

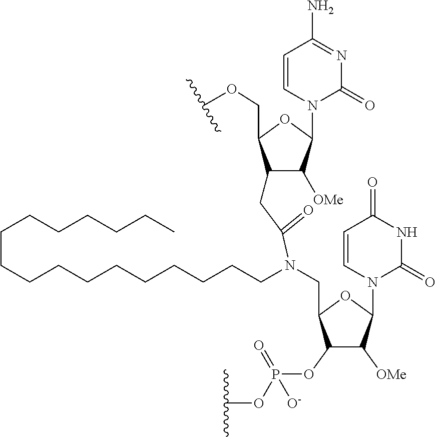

attached at the third and fourth nucleoside from the 5′ end of the sense strand. The compound RD2894 has a C18 lipid conjugate attached to the 5′ end of the sense strand and has the following structure:

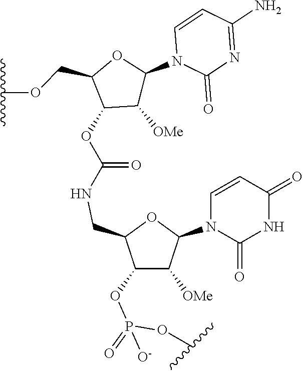 [SMILES: *OC[C@H]1O[C@@H](n2ccc(N)nc2=O)[C@H](OC)[C@@H]1OC(=O)NC[C@H]1O[C@@H](n2ccc(=O)[nH]c2=O)[C@H](OC)[C@@H]1OP(=O)([O-])O*]

attached at the third and fourth nucleoside from the 5′ end of the sense strand.

The compound RD2895 has the following structure:

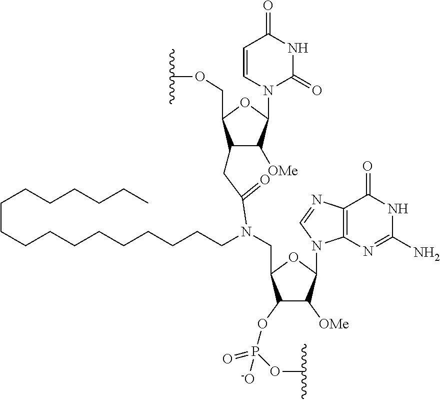

attached at the 4^{th} and 5^{th} nucleoside from the 5′end on the sense strand. The compound RD2896 has the following structure:

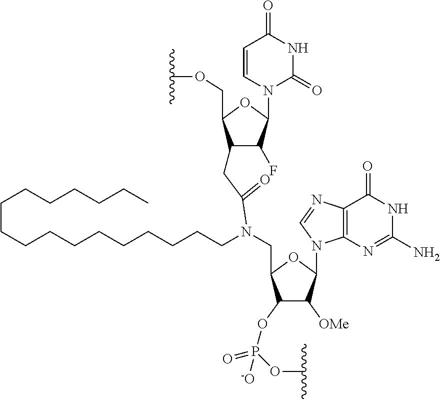

attached at the 7^{th} and 8th nucleoside from the 5′end on the sense strand. The compound has the following structure:

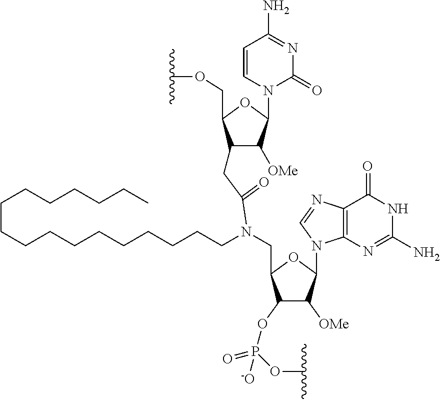

attached at the 17^{th} and 18th nucleoside from the 5′end on the sense strand.

The compound RD2861, RD2854, RD2894, RD2895, RD2896 and RD2897 were evaluated in an in vivo rat PD study as described in Example 103. RNA Isolation and qPCR was performed as described in Example 103. Results are presented in the table below as percent inhibition of CTNNB1, relative to vehicle control.

TABLE 9

| | | | Ref ID |
|---|---|---|---|
| Compound | Backbone | Modified Strands (5′-3′) | NO: |
| RD2861 | mCëmU | (vU)*(fU)*(mU)(mC)(mG)(fA)(mA)(fU)(fC)(mA)(mA) | IA0883 |
| | NB-117 | (mU)(mC)(fC)(mA)(fA)(mC)(mA)(mG)(mU)(mA)*(mG)* | |
| | | (mC) | |
| | | (B7)*(mU)*(mA)(mCëmU))(mG)(mU)(fU)(mG)(fG)(fA) | IS1271 |
| | | (fU)(mU)(mG)(mA)(mU)(mU)(mC)(mG)(mA)(mA)* | |
| | | (mA)*(dQ) | |
| RD2854 | mCëmU | (vU)*(fU)*(mU)(mC)(mG)(fA)(mA)(fU)(fC)(mA)(mA) | IA0883 |
| | NB-104 | (mU)(mC)(fC)(mA)(fA)(mC)(mA)(mG)(mU)(mA)*(mG)* | |
| | | (mC) | |
| | | (B7)*(mU)*(mA)(mCëmU)(mG)(mU)(fU)(mG)(fG)(fA) | IS1264 |
| | | (fU)(mU)(mG)(mA)(mU)(mU)(mC)(mG)(mA)(mA)* | |
| | | (mA)*(dQ) | |
| RD2894 | mCâmU | (vU)*(fU)*(mU)(mC)(mG)(fA)(mA)(fU)(fC)(mA)(mA) | IA0883 |
| | NB-107 | (mU)(mC)(fC)(mA)(fA)(mC)(mA)(mG)(mU)(mA)*(mG)* | |
| | | (mC) | |
| | | (B7)*(mU)*(mA)(mCâmU)(mG)(mU)(fU)(mG)(fG)(fA) | IS1273 |
| | | (fU)(mU)(mG)(mA)(mU)(mU)(mC)(mG)(mA)(mA)* | |
| | | (mA)*(dQ) | |
| RD2895 | mUëmG | (vU)*(fU)*(mU)(mC)(mG)(fA)(mA)(fU)(fC)(mA)(mA) | IA0883 |
| | NB-126 | (mU)(mC)(fC)(mA)(fA)(mC)(mA)(mG)(mU)(mA)*(mG)* | |
| | | (mC) | |
| | | (mU)*(mA)*(mC)(mUëmG)(mU)(fU)(mG)(fG)(fA)(fU) | IS1274 |
| | | (mU)(mG)(mA)(mU)(mU)(mC)(mG)(mA)(mA)*(mA)* | |
| | | (dQ) | |
| RD2896 | fUëmG | (vU)*(fU)*(mU)(mC)(mG)(fA)(mA)(fU)(fC)(mA)(mA) | IA0883 |
| | NB-127 | (mU)(mC)(fC)(mA)(fA)(mC)(mA)(mG)(mU)(mA)*(mG)* | |
| | | (mC) | |
| | | (mU)*(mA)*(mC)(mU)(mG)(mU)(fUëmG)(fG)(fA)(fU) | IS1275 |
| | | (mU)(mG)(mA)(mU)(mU)(mC)(mG)(mA)(mA)*(mA)* | |
| | | (dQ) | |
| RD2897 | mCëmG | (vU)*(fU)*(mU)(mC)(mG)(fA)(mA)(fU)(fC)(mA)(mA) | IA0883 |
| | NB-128 | (mU)(mC)(fC)(mA)(fA)(mC)(mA)(mG)(mU)(mA)*(mG)* | |
| | | (mC) | |
| | | (mU)*(mA)*(mC)(mU)(mG)(mU)(fU)(mG)(fG)(fA)(fU) | IS1276 |
| | | (mU)(mG)(mA)(mU)(mU)(mCëmG)(mA)(mA)*(mA)* | |
| | | (dQ) | |

TABLE 10
Average CTNNB1 Inhibition

Days

| Compound | 15 | 29 |
|---|---|---|
| RD2861 | 41 | 37 |
| RD2854 | 51 | 56 |
| RD2894 | 30 | 19 |
| RD2895 | 58 | 59 |
| RD2896 | 58 | 59 |
| RD2897 | 42 | 23 |

### Example 106: Effect of RD2543 and RD2960 Targeting Rat CTNNB1

The compound RD2543 has a C18 lipid conjugate attached to the 5′ end of the sense strand. RD2960 has the following structure:

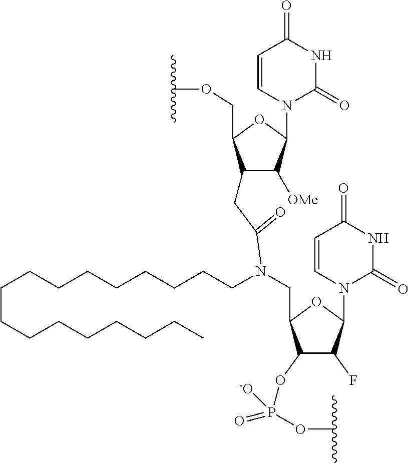

attached at the 6^{th} and 7^{th} nucleoside from the 5′end on the sense strand and the following structure:

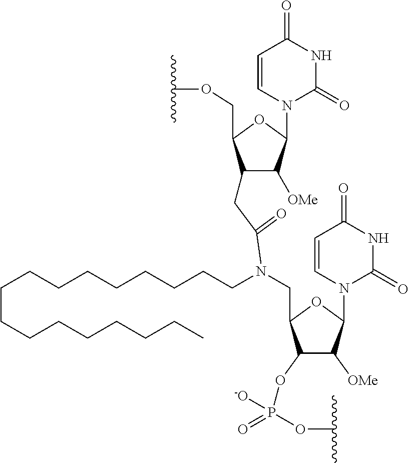

attached at the 15^{th} and 16^{th} nucleoside from the 5′ end on the sense strand. RD2543 and RD2960 were evaluated in an in vivo rat PD study as described in Example 103. Results are presented in the table below as percent inhibition of CTNNB1, relative to vehicle control.

TABLE 11

| | | | Ref ID |
|---|---|---|---|
| Compound | Backbone | Modified Strands (5′-3′) | NO: |
| RD2543 | N/A | (vU)*(fU)*(mU)(mC)(mG)(fA)(mA)(fU)(fC)(mA) | IA0883 |
| | | (mA)(mU)(mC)(fC)(mA)(fA)(mC)(mA)(mG)(mU) | |
| | | (mA)*(mG)*(mC) | |
| | | (B7)*(mU)*(mA)(mC)(mU)(mG)(mU)(fU)(mG) | IS1099 |
| | | (fG)(fA)(fU)(mU)(mG)(mA)(mU)(mU)(mC)(mG) | |
| | | (mA)(mA)*(mA)*(dQ) | |
| RD2960 | mUëmU | (vU)*(fU)*(mU)(mC)(mG)(fA)(mA)(fU)(fC)(mA) | IA0883 |
| | NB-114/ | (mA)(mU)(mC)(fC)(mA)(fA)(mC)(mA)(mG)(mU) | |
| | | (mA)*(mG)*(mC) | |
| | mUëfU | (mU)*(mA)*(mC)(mU)(mG)(mUëfU)(mG)(fG) | IS1292 |
| | NB-116 | (fA)(fU)(mU)(mG)(mA)(mUëmU)(mC)(mG)(mA) | |
| | | (mA)*(mA)*(dQ) | |

TABLE 12
Average CTNNB1 Inhibition

Days

| Compound | 15 | 29 |
|---|---|---|
| RD2543 | 37 | 37 |
| RD2960 | 55 | 56 |

### Example 107: Effect of RD3063 and RD3064 Targeting Rat SOD1 in Various Brain Regions

RD3063 has the following structure:

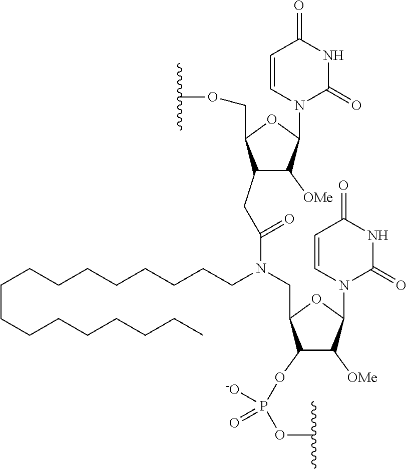

attached at the 5^{th} and 6^{th} nucleoside from the 5′ end on the sense strand and is further described below in Table 13. RD3064 has the following structure:

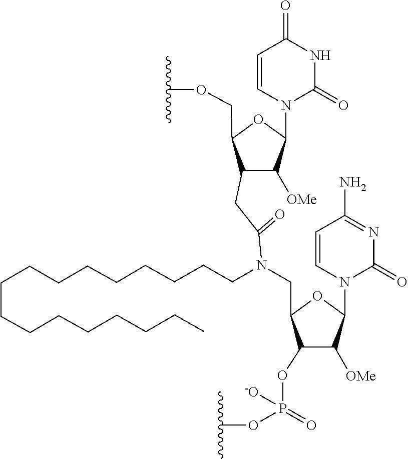

attached at the 12^{th} and 13^{th} nucleoside from the 5′ end on the sense strand and is further described below in Table 13.

The compounds RD3063 and RD3064 were evaluated in an in vivo rat PD study as described in Example 103 with replacement of the CTNNB1 Gene Expression Assay with SOD1 Gene Expression Assay. Three animals per group were dosed and brain regions were collected on day 15. Results are presented in the table below as percent inhibition of SOD1, relative to vehicle control.

TABLE 13

| | | | Ref ID |
|---|---|---|---|
| Compound | Backbone | Modified Strands (5′-3′) | NO: |
| RD3063 | mUëmU | (vU)*(fU)*(mU)(mA)(mG)(fA)(mG)(fU)(fG)(mA) | IA1096 |
| | NB-114 | (mG)(mG)(mA)(fU)(mU)(fA)(mA)(mA)(mA)(mU) | |
| | | (mG)*(mA)*(mG) | |
| | | (AC6)*(mC)*(mA)*(mU)(mU)(mUëmU)(fA)(mA) | IS1332 |
| | | (fU)(fC)(fC)(mU)(mC)(mA)(mC)(mU)(mC)(mU) | |
| | | (mA)*(mA)*(mA) | |
| RD3064 | mUëmC | (vU)*(fU)*(mU)(mA)(mG)(fA)(mG)(fU)(fG)(mA) | IA1096 |
| | NB-115 | (mG)(mG)(mA)(fU)(mU)(fA)(mA)(mA)(mA)(mU) | |
| | | (mG)*(mA)*(mG) | |
| | | (AC6)*(mC)*(mA)*(mU)(mU)(mU)(mU)(fA)(mA) | IS1333 |
| | | (fU)(fC)(fC)(mUëmC)(mA)(mC)(mU)(mC)(mU) | |
| | | (mA)*(mA)*(mA) | |

TABLE 14
Reduction of SOD1 in rats

| | |
|---|---|
| | Average SOD1 Inhibition at Day 15 |
| Brain | (% inhibition relative to control) |

| Region | RD3063 | RD3064 |
|---|---|---|
| Lumbar spinal cord | 91 | 85 |
| Thoracic spinal cord | 90 | 90 |
| Cervical spinal cord | 90 | 90 |
| Brainstem | 92 | 92 |
| Cerebellum | 87 | 81 |
| Hippocampus | 88 | 74 |
| Temporal Cortex | 76 | 73 |
| Frontal Cortex | 91 | 83 |
| Striatum | 56 | 49 |

### Example 108: Effect of RD3063 and RD3064 Targeting Rat SOD1 in Various Brain Regions

The compounds RD3063 and RD3064 described above were evaluated in an in vivo rat PD study as described in Example 103 with replacement of the CTNNB1 Gene Expression Assay with SOD1 Gene Expression Assay. Three animals per group were dosed and brain regions were collected on day 15, 29, 43, 57, 73, 83, 98, 126 and 168. Results are presented in the table below as percent inhibition of SOD1, relative to vehicle control.

TABLE 15A
Reduction of SOD1 in rats

| Compound | D15 LUMB | D15 THOR | D15 CERV | D15 BS |
|---|---|---|---|---|
| RD3063 | 91 | 90 | 90 | 92 |
| RD3064 | 85 | 90 | 90 | 92 |
| Compound | D29 LUMB | D29 THOR | D29 CERV | D29 BS |
| RD3063 | 89 | 87 | 90 | 93 |
| RD3064 | 81 | 86 | 87 | 91 |
| Compound | D43 LUMB | D43 THOR | D43 CERV | D43 BS |
| RD3063 | 93 | 94 | 95 | 96 |
| RD3064 | 80 | 85 | 90 | 93 |
| Compound | D57 LUMB | D57 THOR | D57 CERV | D57 BS |
| RD3063 | 91 | 90 | 94 | 89 |
| Compound | D73 LUMB | D73 THOR | D73 CERV | D73 BS |
| RD3063 | 89 | 93 | 91 | 75 |
| Compound | D83 LUMB | D83 THOR | D83 CERV | D83 BS |
| RD3063 | 70 | 76 | 84 | 78 |
| Compound | D98 LUMB | D98 THOR | D98 CERV | D98 BS |
| RD3063 | 85 | 86 | 87 | 81 |
| Compound | D126 LUMB | D126 THOR | D126 CERV | D126 BS |
| RD3063 | 82 | 83 | 86 | 73 |
| Compound | D168 LUMB | D168 THOR | D168 CERV | D168 BS |
| RD3063 | 85 | 77 | 77 | 54 |

TABLE 15B
Reduction of SOD1 in rats

| | D15 | D15 | D15 | D15 | D15 |
|---|---|---|---|---|---|
| Compound | CBM | HPC | TCtx | CTX | STR |
| RD3063 | 87 | 88 | 76 | 91 | 56 |
| RD3064 | 81 | 74 | 73 | 83 | 49 |
| | D29 | D29 | D29 | D29 | D29 |
| Compound | CBM | HPC | TCtx | CTX | STR |
| RD3063 | 88 | 91 | 91 | 87 | 52 |
| RD3064 | 78 | 83 | 77 | 80 | 62 |
| | D43 | D43 | D43 | D43 | D43 |
| Compound | CBM | HPC | TCtx | CTX | STR |
| RD3063 | 89 | 93 | 92 | 93 | 73 |
| RD3064 | 74 | 80 | 80 | 85 | 38 |
| | D57 | D57 | D57 | D57 | D57 |
| Compound | CBM | HPC | TCtx | CTX | STR |
| RD3063 | 82 | 72 | 84 | 83 | 50 |
| | D73 | D73 | D73 | D73 | D73 |
| Compound | CBM | HPC | TCtx | CTX | STR |
| RD3063 | 86 | 91 | 90 | 76 | 29 |
| | D83 | D83 | D83 | D83 | D83 |
| Compound | CBM | HPC | TCtx | CTX | STR |
| RD3063 | 72 | 81 | 87 | 80 | 49 |
| | D98 | D98 | D98 | D98 | D98 |
| Compound | CBM | HPC | TCtx | CTX | STR |
| RD3063 | 82 | 75 | 81 | 67 | 54 |
| | D126 | D126 | D126 | D126 | D126 |
| Compound | CBM | HPC | TCtx | CTX | STR |
| RD3063 | 76 | 50 | 72 | 42 | 35 |
| | D168 | D168 | D168 | D168 | D168 |
| Compound | CBM | HPC | TCtx | CTX | STR |
| RD3063 | 71 | 45 | 66 | 41 | 17 |

### Example 109: Effect of RD3062 and RD3070 Targeting Rat SOD1 in Various Brain Regions

The compounds RD3062 and RD3070 were evaluated in an in vivo rat PD study as described in Example 103 with replacement of the CTNNB1 Gene Expression Assay with SOD1 Gene Expression Assay. Three animals per group were dosed and brain regions were collected on day 15, 29 and 43. Results are presented in the table below as percent inhibition of SOD1, relative to vehicle control.

TABLE 16

| | | | Ref ID |
|---|---|---|---|
| Compound | Backbone | Modified Strands (5′-3′) | NO: |
| RD3062 | mUëmU | (vU)*(fU)*(mU)(mA)(mG)(fA)(mG)(fU)(fG)(mA) | IA1096 |
| | NB-114 | (mG)(mG)(mA)(fU)(mU)(fA)(mA)(mA)(mA)(mU) | |
| | | (mG)*(mA)*(mG) | |
| | | (AC6)*(mC)*(mA)*(mU)(mUëmU)(mU)(fA)(mA) | IS1331 |
| | | (fU)(fC)(fC)(mU)(mC)(mA)(mC)(mU)(mC)(mU) | |
| | | (mA)*(mA)*(mA) | |
| RD3070 | mUömU | (vU)*(fU)*(mU)(mA)(mG)(fA)(mG)(fU)(fG)(mA) | IA1096 |
| | NB134 | (mG)(mG)(mA)(fU)(mU)(fA)(mA)(mA)(mA)(mU) | |
| | | (mG)*(mA)*(mG) | |
| | | (AC6)*(mC)*(mA)*(mU)(mUömU)(mU)(fA)(mA) | IS1355 |
| | | (fU)(fC)(fC)(mU)(mC)(mA)(mC)(mU)(mC)(mU) | |
| | | (mA)*(mA)*(mA) | |

TABLE 17A
Reduction of SOD1 in rats

| Compound | D15 LUMB | D15 THOR | D15 CERV | D15 BS |
|---|---|---|---|---|
| RD3062 | 90 | 90 | 88 | 91 |
| RD3070 | 87 | 84 | 85 | 85 |
| Compound | D29 LUMB | D29 THOR | D29 CERV | D29 BS |
| RD3062 | 91 | 86 | 90 | 91 |
| RD3070 | 95 | 91 | 90 | 90 |
| Compound | D43 LUMB | D43 THOR | D43 CERV | D43 BS |
| RD3070 | 82 | 85 | 85 | 83 |

TABLE 17B
Reduction of SOD1 in rats

| Compound | D15 CBM | D15 HPC | D15 TCtx | D15 CTX | D15 STR |
|---|---|---|---|---|---|
| RD3062 | 84 | 85 | 82 | 82 | 64 |
| RD3070 | 83 | 81 | 85 | 75 | 50 |
| Compound | D29 CBM | D29 HPC | D29 TCtx | D29 CTX | D29 STR |
| RD3062 | 81 | 83 | 73 | 81 | 38 |
| RD3070 | 82 | 89 | 87 | 85 | 54 |
| Compound | D43 CBM | D43 HPC | D43 TCtx | D43 CTX | D43 STR |
| RD3070 | 71 | 84 | | 82 | 57 |

### Example 110: Effect of RD3071, RD3059 and RD3060 targeting rat SOD1 in various brain regions

The compounds RD3071, RD3059 and RD3060 were evaluated in an in vivo rat PD study as described in Example 103 with replacement of the CTNNB1 Gene Expression Assay with SOD1 Gene Expression Assay. Three animals per group were dosed and brain regions were collected on day 15, 29 and 43. Results are presented in the table below as percent inhibition of SOD1, relative to vehicle control.

TABLE 18

| | | | Ref ID |
|---|---|---|---|
| Compound | Backbone | Modified Strands (5′-3′) | NO: |
| RD3071 | mUömU | (vU)*(fU)*(mU)(mA)(mG)(fA)(mG)(fU)(fG)(mA) | IA1096 |
| | NB-134 | (mG)(mG)(mA)(fU)(mU)(fA)(mA)(mA)(mA)(mU) | |
| | | (mG)*(mA)*(mG) | |
| | | (AC6)*(mC)*(mA)*(mU)(mU)(mUömU)(fA)(mA) | IS1356 |
| | | (fU)(fC)(fC)(mU)(mC)(mA)(mC)(mU)(mC)(mU) | |
| | | (mA)*(mA)*(mA) | |
| RD3059 | mCëmU | (vU)*(fU)*(mU)(mA)(mG)(fA)(mG)(fU)(fG)(mA) | IA1096 |
| | NB-104 | (mG)(mG)(mA)(fU)(mU)(fA)(mA)(mA)(mA)(mU) | |
| | | (mG)*(mA)*(mG) | |
| | | (AC6)*(mC)*(mA)*(mU)(mU)(mU)(mU)(fA)(mA) | IS1328 |
| | | (fU)(fC)(fC)(mU)(mC)(mA)(mCëmU)(mC)(mU) | |
| | | (mA)*(mA)*(mA) | |
| RD3060 | mCëmU | (vU)*(fU)*(mU)(mA)(mG)(fA)(mG)(fU)(fG)(mA) | IA1096 |
| | NB-104 | (mG)(mG)(mA)(fU)(mU)(fA)(mA)(mA)(mA)(mU) | |
| | | (mG)*(mA)*(mG) | |
| | | (AC6)*(mC)*(mA)*(mU)(mU)(mU)(mU)(fA)(mA) | IS1329 |
| | | (fU)(fC)(fC)(mU)(mC)(mA)(mC)(mU)(mCëmU) | |
| | | (mA)*(mA)*(mA) | |

TABLE 19A
Reduction of SOD1 in rats

| | D15 | D15 | D15 | D15 | D15 | D15 | D15 | D15 | D15 |
|---|---|---|---|---|---|---|---|---|---|
| Compound | LUMB | THOR | CERV | BS | CBM | HPC | TCtx | CTX | STR |
| RD3071 | 89 | 82 | 89 | 88 | 86 | 72 | 81 | 78 | 64 |
| RD3059 | 90 | 93 | 93 | 92 | 93 | 87 | 89 | 80 | 71 |
| RD3060 | 86 | 95 | 92 | 84 | 88 | 70 | 82 | 65 | 74 |
| | D29 | D29 | D29 | D29 | D29 | D29 | D29 | D29 | D29 |
| Compound | LUMB | THOR | CERV | BS | CBM | HPC | TCtx | CTX | STR |
| RD3071 | 90 | 91 | 90 | 90 | 78 | 64 | 83 | 79 | 30 |
| RD3059 | 94 | 94 | 92 | 92 | 91 | 68 | 93 | 86 | 53 |
| RD3060 | 90 | 93 | 90 | 93 | 87 | 66 | 91 | 80 | 57 |
| | D43 | D43 | D43 | D43 | D43 | D43 | D43 | D43 | D43 |
| Compound | LUMB | THOR | CERV | BS | CBM | HPC | TCtx | CTX | STR |
| RD3071 | 79 | 85 | 89 | 89 | 84 | 81 | 85 | 69 | 51 |
| RD3059 | 74 | 86 | 93 | 94 | 85 | 81 | 88 | 82 | 67 |
| RD3060 | 84 | 86 | 93 | 92 | 87 | 76 | 83 | 80 | 58 |

### Example 111: Effect of RD3061, RD3147, RD3605, RD3150, RD3604, and RD3639 Targeting Rat SOD1 in Various Brain Regions

The compounds RD3061, RD3147, RD3605, RD3150, RD3604, and RD3639 were evaluated in an in vivo rat PD study as described in Example 103 with replacement of the CTNNB1 Gene Expression Assay with SOD1 Gene Expression Assay. Three animals per group were dosed and brain regions were collected on day 15. Results are presented in the table below as percent inhibition of SOD1, relative to vehicle control.

TABLE 20

| | | | Ref ID |
|---|---|---|---|
| Compound | Backbone | Modified Strands (5′-3′) | NO: |
| RD3061 | mUëmU | (vU)*(fU)*(mU)(mA)(mG)(fA)(mG)(fU)(fG)(mA) | IA1096 |
| | NB-114 | (mG)(mG)(mA)(fU)(mU)(fA)(mA)(mA)(mA)(mU) | |
| | | (mG)*(mA)*(mG) | |
| | | (AC6)*(mC)*(mA)*(mUëmU)(mU)(mU)(fA)(mA) | IS1330 |
| | | (fU)(fC)(fC)(mU)(mC)(mA)(mC)(mU)(mC)(mU) | |
| | | (mA)*(mA)*(mA) | |
| RD3147 | mUëfA | (vU)*(fU)*(mU)(mA)(mG)(fA)(mG)(fU)(fG)(mA) | IA1096 |
| | NB-136 | (mG)(mG)(mA)(fU)(mU)(fA)(mA)(mA)(mA)(mU) | |
| | | (mG)*(mA)*(mG) | |
| | | (AC6)*(mC)*(mA)*(mU)(mU)(mU)(mUëfA)(mA) | IS1418 |
| | | (fU)(fC)(fC)(mU)(mC)(mA)(mC)(mU)(mC)(mU) | |
| | | (mA)*(mA)*(mA) | |
| RD3605 | fAëmA | (vU)*(fU)*(mU)(mA)(mG)(fA)(mG)(fU)(fG)(mA) | IA1096 |
| | NB-142 | (mG)(mG)(mA)(fU)(mU)(fA)(mA)(mA)(mA)(mU) | |
| | | (mG)*(mA)*(mG) | |
| | | (AC6)*(mC)*(mA)*(mU)(mU)(mU)(mU)(fAëmA) | IS1432 |
| | | (fU)(fC)(fC)(mU)(mC)(mA)(mC)(mU)(mC)(mU) | |
| | | (mA)*(mA)*(mA) | |
| RD3150 | mAëfU | (vU)*(fU)*(mU)(mA)(mG)(fA)(mG)(fU)(fG)(mA) | IA1096 |
| | NB-138 | (mG)(mG)(mA)(fU)(mU)(fA)(mA)(mA)(mA)(mU) | |
| | | (mG)*(mA)*(mG) | |
| | | (AC6)*(mC)*(mA)*(mU)(mU)(mU)(mU)(fA) | IS1421 |
| | | (mAëfU)(fC)(fC)(mU)(mC)(mA)(mC)(mU)(mC)(mU) | |
| | | (mA)*(mA)*(mA) | |
| RD3604 | fUëfC | (vU)*(fU)*(mU)(mA)(mG)(fA)(mG)(fU)(fG)(mA) | IA1096 |
| | NB-141 | (mG)(mG)(mA)(fU)(mU)(fA)(mA)(mA)(mA)(mU) | |
| | | (mG)*(mA)*(mG) | |
| | | (AC6)*(mC)*(mA)*(mU)(mU)(mU)(mU)(fA)(mA) | IS1430 |
| | | (fUëfC)(fC)(mU)(mC)(mA)(mC)(mU)(mC)(mU) | |
| | | (mA)*(mA)*(mA) | |
| RD3639 | fCëfC | (vU)*(fU)*(mU)(mA)(mG)(fA)(mG)(fU)(fG)(mA) | IA1096 |
| | NB-147 | (mG)(mG)(mA)(fU)(mU)(fA)(mA)(mA)(mA)(mU) | |
| | | (mG)*(mA)*(mG) | |
| | | (AC6)*(mC)*(mA)*(mU)(mU)(mU)(mU)(fA)(mA) | IS1440 |
| | | (fU)(fCëfC)(mU)(mC)(mA)(mC)(mU)(mC)(mU) | |
| | | (mA)*(mA)*(mA) | |

TABLE 21
Reduction of SOD1 in rats

| Compound | D15 HPC | D15 STR |
|---|---|---|
| RD3061 | 83 | 80 |
| RD3147 | 93 | 70 |
| RD3605 | 91 | 76 |
| RD3150 | 91 | 69 |
| RD3604 | | 57 |
| RD3639 | 75 | 47 |

### Example 112: Effect of RD3069, RD3630, RD3640, RD3065, RD3629, RD3148, RD3149, RD3066, and RD3067 targeting rat SOD1 in various brain regions

The compounds of RD3069, RD3630, RD3640, RD3065, RD3629, RD3148, RD3149, RD3066, and RD3067 were evaluated in an in vivo rat PD study as described in Example 103 with replacement of the CTNNB1 Gene Expression Assay with SOD1 Gene Expression Assay. Three animals per group were dosed and brain regions were collected on day 15. Results are presented in the table below as percent inhibition of SOD1, relative to vehicle control.

TABLE 22

| | | | Ref ID |
|---|---|---|---|
| Compound | Backbone | Modified Strands (5′-3′) | NO: |
| RD3069 | fCëmU | (vU)*(fU)*(mU)(mA)(mG)(fA)(mG)(fU)(fG)(mA) | IA1096 |
| | NB-129 | (mG)(mG)(mA)(fU)(mU)(fA)(mA)(mA)(mA)(mU) | |
| | | (mG)*(mA)*(mG) | |
| | | (AC6)*(mC)*(mA)*(mU)(mU)(mU)(mU)(fA) | IS1354 |
| | | (mA)(fU)(fC)(fCëmU)(mC)(mA)(mC)(mU)(mC)(mU) | |
| | | (mA)*(mA)*(mA) | |
| RD3630 | mCëmA | (vU)*(fU)*(mU)(mA)(mG)(fA)(mG)(fU)(fG)(mA) | IA1096 |
| | NB-145 | (mG)(mG)(mA)(fU)(mU)(fA)(mA)(mA)(mA)(mU) | |
| | | (mG)*(mA)*(mG) | |
| | | (AC6)*(mC)*(mA)*(mU)(mU)(mU)(mU)(fA) | IS1435 |
| | | (mA)(fU)(fC)(fC)(mU)(mCëmA)(mC)(mU)(mC)(mU) | |
| | | (mA)*(mA)*(mA) | |
| RD3640 | mAëmC | (vU)*(fU)*(mU)(mA)(mG)(fA)(mG)(fU)(fG)(mA) | IA1096 |
| | NB-146 | (mG)(mG)(mA)(fU)(mU)(fA)(mA)(mA)(mA)(mU) | |
| | | (mG)*(mA)*(mG) | |
| | | (AC6)*(mC)*(mA)*(mU)(mU)(mU)(mU)(fA) | IS1441 |
| | | (mA)(fU)(fC)(fC)(mU)(mC)(mAëmC)(mU)(mC)(mU) | |
| | | (mA)*(mA)*(mA) | |
| RD3065 | mUëmC | (vU)*(fU)*(mU)(mA)(mG)(fA)(mG)(fU)(fG)(mA) | IA1096 |
| | NB-115 | (mG)(mG)(mA)(fU)(mU)(fA)(mA)(mA)(mA)(mU) | |
| | | (mG)*(mA)*(mG) | |
| | | (AC6)*(mC)*(mA)*(mU)(mU)(mU)(mU)(fA) | IS1334 |
| | | (mA)(fU)(fC)(fC)(mU)(mC)(mA)(mC)(mUëmC)(mU) | |
| | | (mA)*(mA)*(mA) | |
| RD3629 | mUëmA | (vU)*(fU)*(mU)(mA)(mG)(fA)(mG)(fU)(fG)(mA) | IA1096 |
| | NB-144 | (mG)(mG)(mA)(fU)(mU)(fA)(mA)(mA)(mA)(mU) | |
| | | (mG)*(mA)*(mG) | |
| | | (AC6)*(mC)*(mA)*(mU)(mU)(mU)(mU)(fA) | IS1434 |
| | | (mA)(fU)(fC)(fC)(mU)(mC)(mA)(mC)(mU)(mC) | |
| | | (mUëmA)*(mA)*(mA) | |
| RD3148 | mAëmA | (vU)*(fU)*(mU)(mA)(mG)(fA)(mG)(fU)(fG)(mA) | IA1096 |
| | NB-137 | (mG)(mG)(mA)(fU)(mU)(fA)(mA)(mA)(mA)(mU) | |
| | | (mG)*(mA)*(mG) | |
| | | (AC6)*(mC)*(mA)*(mU)(mU)(mU)(mU)(fA) | IS1419 |
| | | (mA)(fU)(fC)(fC)(mU)(mC)(mA)(mC)(mU)(mC)(mU) | |
| | | (mAëmA)*(mA) | |
| RD3149 | mAëmA | (vU)*(fU)*(mU)(mA)(mG)(fA)(mG)(fU)(fG)(mA) | IA1096 |
| | NB-137 | (mG)(mG)(mA)(fU)(mU)(fA)(mA)(mA)(mA)(mU) | |
| | | (mG)*(mA)*(mG) | |
| | | (AC6)*(mC)*(mA)*(mU)(mU)(mU)(mU)(fA) | IS1420 |
| | | (mA)(fU)(fC)(fC)(mU)(mC)(mA)(mC)(mU)(mC)(mU) | |
| | | (mA)*(mAëmA) | |
| RD3066 | mCëmU | (vU)*(fU)*(mU)(mA)(mG)(fA)(mG)(fU)(fG)(mA) | IA1096 |
| | NB-104/ | (mG)(mG)(mA)(fU)(mU)(fA)(mA)(mA)(mA)(mU) | |
| | | (mG)*(mA)*(mG) | |
| | mUëmU | (AC6)*(mC)*(mA)*(mU)(mU)(mUëmU)(fA)(mA) | IS1335 |
| | NB-114 | (fU)(fC)(fC)(mU)(mC)(mA)(mCëmU)(mC)(mU) | |
| | | (mA)*(mA)*(mA) | |
| RD3067 | mUëmU | (vU)*(fU)*(mU)(mA)(mG)(fA)(mG)(fU)(fG)(mA) | IA1096 |
| | NB-114/ | (mG)(mG)(mA)(fU)(mU)(fA)(mA)(mA)(mA)(mU) | |
| | | (mG)*(mA)*(mG) | |
| | mUëmC | (AC6)*(mC)*(mA)*(mU)(mU)(mUëmU)(fA)(mA) | IS1336 |
| | NB-115 | (fU)(fC)(fC)(mU)(mC)(mA)(mC)(mUëmC)(mU) | |
| | | (mA)*(mA)*(mA) | |

TABLE 23
Reduction of SOD1 in rats

| Compound | D15 HPC | D15 CTX | D15 STR |
|---|---|---|---|
| RD3069 | 40 | 30 | 19 |
| RD3630 | 91 | 90 | 71 |
| RD3640 | 91 | 87 | 69 |
| RD3065 | 90 | 90 | 77 |
| RD3629 | 87 | 74 | 61 |
| RD3148 | 89 | 81 | 77 |
| RD3149 | 84 | 68 | 70 |
| RD3066 | 86 | 78 | 66 |
| RD3067 | 87 | 72 | 64 |

### Example 113: Effect of Compound 1 and Compound 2 Targeting Target a in Various Brain Regions

Compound 1 and compound 2 were evaluated in an in vivo human Target A transgenic mice PD study. The animals received a single vehicle or 0.2 mg (10 mg/kg) dose via intracerebroventricular injection on day 1 (n=3/group). Animals were observed every day for behavioral changes. Brain regions were collected on day 8, 15, 21 and 29, and tissue was immediately placed in homogenizing tube, snap frozen, then kept at −80° C. for gene expression analysis.

RNA Isolation and qPCR was performed as described in Example 103, with the exceptions that the instead of rat CTNNB1 TaqMan Gene Expression Assay, the human Target A TaqMan Gene Expression Assay (Thermo Fisher) was used; instead of rat ACTB (VIC) TaqMan Gene Expression Assay, the mouse GAPDH TaqMan Gene Expression Assay (Thermo Fisher: Mm99999915_g1, VIC) was used. Results are presented in the Table below as percent inhibition of Target A, relative to vehicle control.

TABLE 24

| | | | Ref ID |
|---|---|---|---|
| Compound | Backbone | Modified Strands (5′-3′) | NO: |
| 1 | mAëmU | (vX)*(fX)*(mX)(fX)(mX)(fX)(mX)(fX)(mX)(fX) | IA1 |
| | NB-165 | (mX)(fX)(mX)(fX)(mX)(fX)(mX)(fX)(mX)(fX) | |
| | | (mX)*(mX)*(mX) | |
| | | (AC6)*(mX)*(mX)(mX)(mXëmX)(mX)(fX)(mX) | IS1 |
| | | (fX)(fX)(fX)(fX)(mX)(mX)(mX)(mX)(mX)(mX) | |
| | | (mX)(mX)*(mX)*(dQ) | |
| 2 | mUëfC | (vX)*(fX)*(mX)(fX)(mX)(fX)(mX)(fX)(mX)(fX) | IA1 |
| | NB-166 | (mX)(fX)(mX)(fX)(mX)(fX)(mX)(fX)(mX)(fX) | |
| | | (mX)*(mX)*(mX) | |
| | | (AC6)*(mX)*(mX)(mX)(mX)(mX)(mX)(fX)(mXëfX) | IS2 |
| | | (fX)(fX)(fX)(mX)(mX)(mX)(mX)(mX)(mX) | |
| | | (mX)(mX)*(mX)*(dQ) | |

TABLE 25
Reduction of Target A

| | D8 | D8 | D8 | D8 | D8 | D8 | D8 | D8 |
|---|---|---|---|---|---|---|---|---|
| Compound | CBM | CTX | HPC | STR | BS | CERV | THOR | LUMB |
| 1 | 31 | 22 | 38 | 30 | 73 | 71 | 72 | 70 |
| 2 | 53 | 47 | 52 | 35 | 74 | 67 | 67 | 64 |

| | D15 | D15 | D15 | D15 | D15 | D15 | D15 | D15 |
|---|---|---|---|---|---|---|---|---|
| Compound | CBM | CTX | HPC | STR | BS | CERV | THOR | LUMB |
| 1 | 31 | 22 | 38 | 30 | 73 | 71 | 72 | 70 |
| 2 | 53 | 47 | 52 | 35 | 74 | 67 | 67 | 64 |

| | C21 | D22 | D22 | D22 | D22 |
|---|---|---|---|---|---|
| Compound | CBM | CTX | HPC | STR | CERV |
| 1 | 32 | 50 | 47 | 41 | 72 |
| 2 | 34 | 53 | 50 | 29 | 74 |
| | C29 | D29 | D29 | D29 | D29 |
| Compound | CBM | CTX | HPC | STR | CERV |
| 1 | 32 | 50 | 47 | 41 | 72 |
| 2 | 34 | 53 | 50 | 29 | 74 |

### Example 114: Effect of Compound 3 Targeting Target a in Various Brain Regions

Compound 3 was evaluated in an in vivo human Target A transgenic mice PD study as described in Example 113. Three animals per group were dosed and brain regions were collected on day 8, 15, 21 and 29. Results are presented in the Table below as percent inhibition of Target A, relative to vehicle control.

TABLE 26

| | | | Ref ID |
|---|---|---|---|
| Compound | Backbone | Modified Strands (5′-3′) | NO: |
| 3 | ê | (vX)*(fX)*(mX)(fX)(mX)(fX)(mX)(fX)(mX)(fX) | IA2 |
| | NB-162/ | (mX)(fX)(mX)(fX)*(mX)*(fXêmX)*(fXêmX)* | |
| | NB-120/ | (fXêmX)*(mX)*(mX) | |
| | NB-160 | (AC6)*(mX)(fX)(fX)(fX)(fX)(mX)(mX)(mX)(mX) | IS3 |
| | | (mX)(mX)(mX)(mX)(mX)*(dQ) | |

TABLE 27
Reduction of Target A

| | D8 | D8 | D8 | D8 | D8 | D8 | D8 | D8 |
|---|---|---|---|---|---|---|---|---|
| Compound | CBM | CTX | HPC | STR | BS | CERV | THOR | LUMB |
| 3 | 25 | 29 | 16 | 11 | 19 | 32 | 22 | 19 |

| | D15 | D15 | D15 | D15 | D15 | D15 | D15 | D15 |
|---|---|---|---|---|---|---|---|---|
| Compound | CBM | CTX | HPC | STR | BS | CERV | THOR | LUMB |
| 3 | 25 | 29 | 16 | 11 | 19 | 32 | 22 | 19 |

| | C21 | D22 | D22 | D22 | D22 |
|---|---|---|---|---|---|
| Compound | CBM | CTX | HPC | STR | CERV |
| 3 | 16 | 12 | 25 | 5 | |
| | C29 | D29 | D29 | D29 | D29 |
| Compound | CBM | CTX | HPC | STR | CERV |
| 3 | 16 | 12 | 25 | 5 | |

### Example 115: Effect of Compound 4, Compound 5, Compound 6 and Compound 7 Targeting Target a in Various Brain Regions

Compound 4, compound 5, compound 6 and compound 7 were evaluated in an in vivo human Target A transgenic mice PD study as described in Example 113. Three animals per group were dosed and brain regions were collected on day 15 and 29. Results are presented in the Table below as percent inhibition of Target A, relative to vehicle control.

TABLE 28

| | | | Ref ID |
|---|---|---|---|
| Compound | Backbone | Modified Strands (5′-3′) | NO: |
| 4 | ê | (vX)*(fX)*(mX)(fX)(mX)(fX)(mX)(fX)(mX)(fX) | IA3 |
| | NB-161/ | (mX)(fX)(mX)(fX)*(mXêfX)*(mXêfX)*(mXêfX)* | |
| | NB-159/ | (mXêmX) *(mX) | |
| | NB-121/ | (AC6)*(mX)(fX)(fX)(fX)(fX)(mX)(mX)(mX)(mX) | IS3 |
| | NB-163 | (mX)(mX)(mX)(mX) (mX)*(dQ) | |
| 5 | ê | (vX)*(fX)*(mX)(fX)(mX)(fX)(mX)(fX)(mX)(fX) | IA4 |
| | NB-159/ | (mX)(fX)(mX)(fX)*(mX)*(fX)*(mXêfX)*(mXêfX)* | |
| | NB-121/ | (mXêmX)*(mX) | |
| | NB-163 | (AC6)*(mX)(fX)(fX)(fX)(fX)(mX)(mX)(mX)(mX) | IS3 |
| | | (mX)(mX)(mX)(mX)(mX)*(dQ) | |
| 6 | ê | (vX)*(fX)*(mX)(fX)(mX)(fX)(mX)(fX)(mX)(fX) | IA3 |
| | NB-161/ | (mX)(fX)(mX)(fX)*(mXêfX)*(mXêfX)*(mXêfX)* | |
| | NB-159/ | (mXemX)*(mX) | |
| | NB-121/ | (AC6)*(mX)(fX)(fX)(fX)(fX)(mXêmX) | IS4 |
| | NB-163/ | (mX)(mXêmX)(mX)(mXêmX)(mX)*(dQ) | |
| | NB-143/ | | |
| | NB-170 | | |
| 7 | ê | (vX)*(fX)*(mX)(fX)(mX)(fX)(mX)(fX)(mX)(fX) | IA4 |
| | NB-159/ | (mX)(fX)(mX)(fX)*(mX)*(fX)*(mXêfX)*(mXêfX)* | |
| | NB-121/ | (mXêmX)*(mX) | |
| | NB-163/ | (AC6)*(mX)(fX)(fX)(fX)(fX)(mXêmX) | IS4 |
| | NB-143/ | (mX)(mXêmX)(mX)(mXêmX)(mX)*(dQ) | |
| | NB-170 | | |

TABLE 29
Reduction of Target A

| | D15 | D15 | D15 | D15 | D15 |
|---|---|---|---|---|---|
| Compound | CBM | CTX | HPC | STR | CERV |
| 4 | 2 | | 10 | 28 | 32 |
| 5 | 15 | | 19 | 25 | 28 |
| 6 | 18 | | 20 | 31 | 29 |
| 7 | 15 | | 13 | 20 | 22 |
| | C29 | D29 | D29 | D29 | D29 |
| Compound | CBM | CTX | HPC | STR | CERV |
| 4 | 9 | 28 | 5 | 25 | 38 |
| 5 | 19 | 9 | 12 | 13 | 35 |
| 6 | 30 | 17 | 9 | 19 | 37 |
| 7 | 18 | 19 | 6 | 23 | 40 |

### Example 116: Effect of Compound 8 Targeting Target a in Various Brain Regions

Compound 8 was evaluated in an in vivo human Target A transgenic mice PD study as described in Example 113. Three animals per group were dosed and brain regions were collected on day 15 and 29. Results are presented in the Table below as percent inhibition of Target A, relative to vehicle control.

TABLE 30

| | | | Ref ID |
|---|---|---|---|
| Compound | Backbone | Modified Strands (5′-3′) | NO: |
| 8 | ê | (vX)*(fX)*(mX)(fX)(mX)(fX)(mX)(fX)(mX)(fX) | IA2 |
| | NB-162/ | (mX)(fX)(mX)(fX)*(mX)*(fXêmX)*(fXêmX)* | |
| | NB-120/ | (fXêmX)*(mX)*(mX) | |
| | NB-160/ | (AC6)*(mX)(fX)(fX)(fX)(fX)(mXêmX) | IS4 |
| | NB-143/ | (mX)(mXêmX)(mX)(mXêmX)(mX)*(dQ) | |
| | NB-170 | | |

TABLE 31
Reduction of Target A

| | D15 | D15 | D15 | D15 | D15 |
|---|---|---|---|---|---|
| Compound | CBM | CTX | HPC | STR | CERV |
| 8 | 12 | 17 | 12 | 4 | 25 |
| | C29 | D29 | D29 | D29 | D29 |
| Compound | CBM | CTX | HPC | STR | CERV |
| 8 | 6 | 1 | | | 6 |

### Example 117: Effect of Compound 9 and Compound 10 Targeting Target a in Various Brain Regions

Compound 9 and compound 10 were evaluated in an in vivo human Target A transgenic mice PD study as described in Example 113. Three animals per group were dosed and brain regions were collected on day 15. Results are presented in the Table below as percent inhibition of Target A, relative to vehicle control.

TABLE 32

| | | | Ref ID |
|---|---|---|---|
| Compound | Backbone | Modified Strands (5′-3′) | NO: |
| 9 | fGëmU | (vX)*(fX)*(mX)(fX)(mX)(fX)(mX)(fX)(mX)(fX) | IA5 |
| | NB-152/ | (mX)(fX)(mX)(fX)(mX)(fX)(mX)(fXêmX)(fX)(mX) | |
| | fUêmG | *(mX)*(mX) | |
| | NB-120 | (AC6)*(mX)*(mX)(mX)(mX)(mX)(mX)(fXëmX) | IS5 |
| | | (fX)(fX)(fX)(fX)(mX)(mX)(mX)(mX)(mX)(mX) | |
| | | (mX)(mX)*(mX)*(dQ) | |
| 10 | fGëmU | (eX)*(fX)*(mX)(fX)(mX)(fX)(mX)(fX)(mX)(fX) | IA6 |
| | NB-152/ | (mX)(fX)(mX)(fX)(mX)(fX)(mX)(fXêmX)(fX)(mX)* | |
| | fUêmG | (mX)*(mX) | |
| | NB-120 | (AC6)*(mX)*(mX)(mX)(mX)(mX)(mX)(fXëmX) | IS5 |
| | | (fX)(fX)(fX)(fX)(mX)(mX)(mX)(mX)(mX)(mX) | |
| | | (mX)(mX)*(mX)*(dQ) | |

TABLE 33
Reduction of Target A

| | D15 | D15 | D15 | D15 | D15 |
|---|---|---|---|---|---|
| Compound | CBM | CTX | HPC | STR | CERV |
| 9 | 61 | 68 | 80 | 77 | 73 |
| 10 | 47 | 55 | 74 | 75 | 73 |

### Example 118: Effect of Compound 11 Targeting Target a in Various Brain Regions

Compound 11 was evaluated in an in vivo human Target A transgenic mice PD study as described in Example 113. Three animals per group were dosed and brain regions were collected on day 15. Results are presented in the Table below as percent inhibition of Target A, relative to vehicle control.

TABLE 34

| | | | Ref ID |
|---|---|---|---|
| Compound | Backbone | Modified Strands (5′-3′) | NO: |
| 11 | fGêmU | (eX)*(fX)*(mXU)(fX)(mX)(fX)(mX)(fX)(mX)(fX) | IA7 |
| | NB-154 | (mX)(fX)(mX)(fX)(mX)(fX)(mX)(fX)(mX)(fX) | |
| | | (mX)*(mX)*(mX) | |
| | | (AC6)*(mX)*(mX)(mX)(mX)(mX)(mX)(fXêmX) | IS6 |
| | | (fX)(fX)(fX)(fX)(mX)(mX)(mX)(mX)(mX)(mX) | |
| | | (mX)(mX)*(mX)*(dQ) | |

TABLE 35
Reduction of Target A

| | D15 | D15 | D15 | D15 | D15 |
|---|---|---|---|---|---|
| Compound | CBM | CTX | HPC | STR | CERV |
| 11 | 30 | 18 | 36 | 31 | 33 |

### Example 119: Effect of Compound 12 Targeting Target a in Various Brain Regions

Compound 12 was evaluated in an in vivo human Target A transgenic mice PD study as described in Example 113. Three animals per group were dosed and brain regions were collected on day 15. Results are presented in the Table below as percent inhibition of Target A, relative to vehicle control.

TABLE 36

| | | | Ref ID |
|---|---|---|---|
| Compound | Backbone | Modified Strands (5′-3′) | NO: |
| 12 | fGëmU | (eX)*(fX)*(mX)(fX)(mX)(fX)(mX)(fX)(mX)(fX) | IA7 |
| | NB-152 | (mX)(fX)(mX)(fX)(mX)(fX)(mX)(fX)(mX)(fX)(mX)* | |
| | | (mX)*(mX) | |
| | | (AC6)*(mX)*(mX)(mX)(mX)(mX)(mX)(fXëmX) | IS7 |
| | | (fX)(fX)(fX)(fX)(mX)(mX)(mX)(mX)(mX)(mX) | |
| | | (mX)(mX)*(mX)*(dQ) | |

TABLE 37
Reduction of Target A

| | D15 | D15 | D15 | D15 | D15 |
|---|---|---|---|---|---|
| Compound | CBM | CTX | HPC | STR | CERV |
| 12 | 80 | 76 | 81 | 72 | 87 |

### Example 120: Effect of Compound 13 and Compound 14 Targeting Target a in Various Brain Regions

Compound 13 and compound 14 were evaluated in an in vivo human Target A transgenic mice PD study as described in Example 113. Three animals per group were dosed and brain regions were collected on day 15. Results are presented in the Table below as percent inhibition of Target A, relative to vehicle control.

TABLE 38

| | | | Ref ID |
|---|---|---|---|
| Compound | Backbone | Modified Strands (5′-3′) | NO: |
| 13 | fG[ë1]mU | (eX)*(fX)*(mX)(fX)(mX)(fX)(mX)(fX)(mX)(fX) | IA7 |
| | NB-188 | (mX)(fX)(mX)(fX)(mX)(fX)(mX)(fX)(mX)(fX)(mX)* | |
| | | (mX)*(mX) | |
| | | (AC6)*(mX)*(mX)(mX)(mX)(mX)(mX) | IS8 |
| | | (fX[ë1]mX)(fX)(fX)(fX)(fX)(mX)(mX)(mX)(mX)(mX) | |
| | | (mX)(mX)(mX)*(mX)*(dQ) | |
| 14 | fG[ë2]mU | (eX)*(fX)*(mX)(fX)(mX)(fX)(mX)(fX)(mX)(fX) | IA7 |
| | NB-189 | (mX)(fX)(mX)(fX)(mX)(fX)(mX)(fX)(mX)(fX)(mX)* | |
| | | (mX)*(mX) | |
| | | (AC6)*(mX)*(mX)(mX)(mX)(mX)(mX) | IS9 |
| | | (fX[ë2]mX)(fX)(fX)(fX)(fX)(mX)(mX)(mX)(mX)(mX) | |
| | | (mX)(mX)(mX)*(mX)*(dQ) | |

TABLE 39
Reduction of Target A

| | D15 | D15 | D15 | D15 | D15 |
|---|---|---|---|---|---|
| Compound | CBM | CTX | HPC | STR | CERV |
| 13 | 25 | 26 | 29 | 31 | 3 |
| 14 | 31 | 27 | 35 | 38 | |

### Example 121: Effect of Compound 15, Compound 16, Compound 17, Compound 18, and Compound 19 Targeting Target a in Various Brain Regions

Compound 15, compound 16, compound 17, compound 18, and compound 19 were evaluated in an in vivo human Target A transgenic mice PD study as described in Example 113. Three animals per group were dosed and brain regions were collected on day 15. Results are presented in the Table below as percent inhibition of Target A, relative to vehicle control.

TABLE 40

| | | | Ref ID |
|---|---|---|---|
| Compound | Backbone | Modified Strands (5′-3′) | NO: |
| 15 | mUëmG | (eX)*(fX)*(mX)(fX)(mX)(fX)(mX)(fX)(mX)(fX) | IA7 |
| | NB-126 | (mX)(fX)(mX)(fX)(mX)(fX)(mX)(fX)(mX)(fX)(mX)* | |
| | | (mX)*(mX) | |
| | | (AC6)*(mX)*(mX)(mX)(mX)(mXëmX)(fX)(mX) | IS10 |
| | | (fX)(fX)(fX)(fX)(mX)(mX)(mX)(mX)(mX)(mX) | |
| | | (mX)(mX)*(mX)*(dQ) | |
| 16 | mAëmU | (eX)*(fX)*(mX)(fX)(mX)(fX)(mX)(fX)(mX)(fX) | IA7 |
| | NB-165 | (mX)(fX)(mX)(fX)(mX)(fX)(mX)(fX)(mX)(fX)(mX)* | |
| | | (mX)*(mX) | |
| | | (AC6)*(mX)*(mX)(mX)(mXëmX)(mX)(fX)(mX) | IS11 |
| | | (fX)(fX)(fX)(fX)(mX)(mX)(mX)(mX)(mX)(mX) | |
| | | (mX)(mX)*(mX)*(dQ) | |
| 17 | mGëfG | (eX)*(fX)*(mX)(fX)(mX)(fX)(mX)(fX)(mX)(fX) | IA7 |
| | NB-180 | (mX)(fX)(mX)(fX)(mX)(fX)(mX)(fX)(mX)(fX)(mX)* | |
| | | (mX)*(mX) | |
| | | (AC6)*(mX)*(mX)(mX)(mX)(mX)(mXëfX)(mX) | IS12 |
| | | (fX)(fX)(fX)(fX)(mX)(mX)(mX)(mX)(mX)(mX) | |
| | | (mX)(mX)*(mX)*(dQ) | |
| 18 | mUëfC | (eX)*(fX)*(mX)(fX)(mX)(fX)(mX)(fX)(mX)(fX) | IA7 |
| | NB-166 | (mX)(fX)(mX)(fX)(mX)(fX)(mX)(fX)(mX)(fX)(mX)* | |
| | | (mX)*(mX) | |
| | | (AC6)*(mX)*(mX)(mX)(mX)(mX)(mX)(fX) | IS13 |
| | | (mXëfX)(fX)(fX)(fX)(mX)(mX)(mX)(mX)(mX)(mX) | |
| | | (mX)(mX)*(mX)*(dQ) | |
| 19 | mAëmA | (eX)*(fX)*(mX)(fX)(mX)(fX)(mX)(fX)(mX)(fX) | IA7 |
| | NB-137 | (mX)(fX)(mX)(fX)(mX)(fX)(mX)(fX)(mX)(fX)(mX)* | |
| | | (mX)*(mX) | |
| | | (AC6)*(mX)*(mX)(mX)(mX)(mX)(mX)(fX)(mX) | IS14 |
| | | (fX)(fX)(fX)(fX)(mXëmX)(mX)(mX)(mX)(mX)(mX) | |
| | | (mX)*(mX)*(dQ) | |

TABLE 41
Reduction of Target A

| | D15 | D15 | D15 | D15 | D15 |
|---|---|---|---|---|---|
| Compound | CBM | CTX | HPC | STR | CERV |
| 15 | 53 | 55 | 63 | 69 | 67 |
| 16 | 63 | 63 | 72 | 73 | 77 |
| 17 | 64 | 51 | 69 | 66 | 69 |
| 18 | 66 | 67 | 73 | 73 | 79 |
| 19 | 69 | 68 | 81 | 76 | 82 |

### Example 122: Effect of Compound 20, Compound 21, Compound 22, and Compound 23 Targeting Target a in Various Brain Regions

Compound 20, compound 21, compound 22, and compound 23 were evaluated in an in vivo human Target A transgenic mice PD study as described in Example 113. Three animals per group were dosed and brain regions were collected on day 15. Results are presented in the Table below as percent inhibition of Target A, relative to vehicle control.

TABLE 42

| | | | Ref ID |
|---|---|---|---|
| Compound | Backbone | Modified Strands (5′-3′) | NO: |
| 20 | mAëmA | (eX)*(fX)*(mX)(fX)(mX)(fX)(mX)(fX)(mX)(fX) | IA7 |
| | NB-137 | (mX)(fX)(mX)(fX)(mX)(fX)(mX)(fX)(mX)(fX)(mX)* | |
| | | (mX)*(mX) | |
| | | (AC6)*(mX)*(mX)(mX)(mX)(mX)(mX)(fX)(mX) | IS15 |
| | | (fX)(fX)(fX)(fX)(mX)(mX)(mXëmX)(mX)(mX)(mX) | |
| | | (mX)*(mX)*(dQ) | |
| 21 | mAëmG | (eX)*(fX)*(mX)(fX)(mX)(fX)(mX)(fX)(mX)(fX) | IA7 |
| | NB-167 | (mX)(fX)(mX)(fX)(mX)(fX)(mX)(fX)(mX)(fX)(mX)* | |
| | | (mX)*(mX) | |
| | | (AC6)*(mX)*(mX)(mX)(mX)(mX)(mX)(fX)(mX) | IS16 |
| | | (fX)(fX)(fX)(fX)(mX)(mX)(mX)(mXëmX)(mX)(mX) | |
| | | (mX)*(mX)*(dQ) | |
| 22 | mGëmC | (eX)*(fX)*(mX)(fX)(mX)(fX)(mX)(fX)(mX)(fX) | IA7 |
| | NB-179 | (mX)(fX)(mX)(fX)(mX)(fX)(mX)(fX)(mX)(fX)(mX)* | |
| | | (mX)*(mX) | |
| | | (AC6)*(mX)*(mX)(mX)(mX)(mX)(mX)(fX)(mX) | IS17 |
| | | (fX)(fX)(fX)(fX)(mX)(mX)(mX)(mX)(mXëmX)(mX) | |
| | | (mX)*(mX)*(dQ) | |
| 23 | mAëmA | (eX)*(fX)*(mX)(fX)(mX)(fX)(mX)(fX)(mX)(fX) | IA7 |
| | NB-137 | (mX)(fX)(mX)(fX)(mX)(fX)(mX)(fX)(mX)(fX)(mX)* | |
| | | (mX)*(mX) | |
| | | (AC6)*(mX)*(mX)(mX)(mX)(mX)(mX)(fX)(mX) | IS18 |
| | | (fX)(fX)(fX)(fX)(mX)(mX)(mX)(mX)(mX)(mX) | |
| | | (mXëmX)*(mX)*(dQ) | |

TABLE 43
Reduction of Target A

| | D15 | D15 | D15 | D15 | D15 |
|---|---|---|---|---|---|
| Compound | CBM | CTX | HPC | STR | CERV |
| 20 | 95 | 68 | 79 | 74 | 91 |
| 21 | 87 | 40 | 44 | 46 | 74 |
| 22 | 95 | 55 | 80 | 81 | 91 |
| 23 | 96 | 60 | 80 | 70 | 75 |

### Example 123: Effect of Compound 24 and Compound 25 Targeting Target a in Various Brain Regions

Compound 24 and compound 25 were evaluated in an in vivo human Target A transgenic mice PD study as described in Example 113. Three animals per group were dosed and brain regions were collected on day 15. Results are presented in the Table below as percent inhibition of Target A, relative to vehicle control.

TABLE 44

| | | | Ref ID |
|---|---|---|---|
| Compound | Backbone | Modified Strands (5′-3′) | NO: |
| 24 | mAëmA | (eX)*(fX)*(mX)(fX)(mX)(fX)(mX)(fX)(mX)(fX) | IA7 |
| | NB-137 | (mX)(fX)(mX)(fX)(mX)(fX)(mX)(fX)(mX)(fX)(mX)* | |
| | | (mX)*(mX) | |
| | | (AC6)*(mX)*(mX)(mX)(mX)(mX)(mX)(fX)(mX) | IS19 |
| | | (fX)(fX)(fX)(fX)(mX)(mXëmX)(mX)(mX)(mX)(mX) | |
| | | (mX)*(mX)*(dQ) | |
| 25 | mCëmA | (eX)*(fX)*(mX)(fX)(mX)(fX)(mX)(fX)(mX)(fX) | IA7 |
| | NB-145 | (mX)(fX)(mX)(fX)(mX)(fX)(mX)(fX)(mX)(fX)(mX)* | |
| | | (mX)*(mX) | |
| | | (AC6)*(mX)*(mX)(mX)(mX)(mX)(mX)(fX)(mX) | IS20 |
| | | (fX)(fX)(fX)(fX)(mX)(mX)(mX)(mX)(mX)(mXëmX) | |
| | | (mX)*(mX)*(dQ) | |

TABLE 45
Reduction of Target A

| | D15 | D15 | D15 | D15 | D15 |
|---|---|---|---|---|---|
| Compound | CBM | CTX | HPC | STR | CERV |
| 24 | 64 | 64 | 72 | 71 | 78 |
| 25 | 67 | 52 | 70 | 71 | 78 |

### Example 124: Effect of Compound 26 and Compound 27 Targeting Target a in Various Brain Regions

Compound 26 and compound 27 were evaluated in an in vivo human Target A transgenic mice PD study as described in Example 113. Three animals per group were dosed and brain regions were collected on day 15. Results are presented in the Table below as percent inhibition of Target A, relative to vehicle control.

TABLE 46

| | | | Ref ID |
|---|---|---|---|
| Compound | Backbone | Modified Strands (5′-3′) | NO: |
| 26 | mAëmA | (eX)*(fX)*(mX)(fX)(mX)(fX)(mX)(fX)(mX)(fX) | IA7 |
| | NB-137/ | (mX)(fX)(mX)(fX)(mX)(fX)(mX)(fX)(mX)(fX)(mX)* | |
| | NB-137 | (mX)*(mX) | |
| | | (AC6)*(mX)*(mX)(mX)(mX)(mX)(mX)(fX)(mX) | IS21 |
| | | (fX)(fX)(fX)(fX)(mXëmX)(mXëmX)(mX)(mX)(mX) | |
| | | (mX)*(mX)*(dQ) | |
| 27 | fGëmU | (eX)*(fX)*(mX)(fX)(mX)(fX)(mX)(fX)(mX)(fX) | IA7 |
| | NB-152/ | (mX)(fX)(mX)(fX)(mX)(fX)(mX)(fX)(mX)(fX)(mX)* | |
| | mAëmA | (mX)*(mX) | |
| | NB-137 | (AC6)*(mX)*(mX)(mX)(mX)(mX)(mX)(fXëmX) | IS22 |
| | | (fX)(fX)(fX)(fX)(mX)(mXëmX)(mX)(mX)(mX)(mX) | |
| | | (mX)*(mX)*(dQ) | |

TABLE 47
Reduction of Target A

| | D15 | D15 | D15 | D15 | D15 |
|---|---|---|---|---|---|
| Compound | CBM | CTX | HPC | STR | CERV |
| 26 | 50 | 51 | 58 | 53 | 66 |
| 27 | 58 | 46 | 65 | 70 | 70 |

### Example 125: Effect of Compound 28 Targeting Target a in Various Brain Regions

Compound 28 was evaluated in an in vivo human Target A transgenic mice PD study as described in Example 113. Three animals per group were dosed and brain regions were collected on day 15. Results are presented in the Table below as percent inhibition of Target A, relative to vehicle control.

TABLE 48

| | | | Ref ID |
|---|---|---|---|
| Compound | Backbone | Modified Strands (5′-3′) | NO: |
| 28 | fGëmU | (eX)*(fX)*(mX)(fX)(mX)(fX)(mX)(fX)(mX)(fX) | IA7 |
| | NB-152/ | (mX)(fX)(mX)(fX)(mX)(fX)(mX)(fX)(mX)(fX)(mX)* | |
| | mAëmA | (mX)*(mX) | |
| | NB-137 | (AC6)*(mX)*(mX)(mX)(mX)(mX)(mX)(fXëmX) | IS23 |
| | | (fX)(fX)(fX)(fX)(mXëmX)(mX)(mX)(mX)(mX)(mX) | |
| | | (mX)*(mX)*(dQ) | |

TABLE 49
Reduction of Target A

| | D15 | D15 | D15 | D15 | D15 |
|---|---|---|---|---|---|
| Compound | CBM | CTX | HPC | STR | CERV |
| 28 | 68 | 65 | | 69 | 76 |

### Example 126: Effect of Compound 29 and Compound 30 Targeting Target a in Various Brain Regions

Compound 29 and compound 30 were evaluated in an in vivo human Target A transgenic mice PD study as described in Example 113. Three animals per group were dosed and brain regions were collected on day 15. Results are presented in the Table below as percent inhibition of Target A, relative to vehicle control.

TABLE 50

| | | | Ref ID |
|---|---|---|---|
| Compound | Backbone | Modified Strands (5′-3′) | NO: |
| 29 | mUëmA | (eX)*(fX)*(mX)(fX)(mX)(fX)(mX)(fX)(mX)(fX) | IA7 |
| | NB-144 | (mX)(fX)(mX)(fX)(mX)(fX)(mX)(fX)(mX)*(fX)* | |
| | | (mX) | |
| | | (AC6)*(mX)*(mX)(mX)(mXëmX)(mX)(fX)(mX) | IS24 |
| | | (fX)(fX)(fX)(fX)(mX)(mX)(mX)(mX)(mX)(mX) | |
| | | (mX)(mX)(mX)*(dQ)*(SC6)(mX) | |
| 30 | mAëmC | (eX)*(fX)*(mX)(fX)(mX)(fX)(mX)(fX)(mX)(fX) | IA7 |
| | NB-146 | (mX)(fX)(mX)(fX)(mX)(fX)(mX)(fX)(mX)*(fX)* | |
| | | (mX) | |
| | | (AC6)*(mX)*(mX)(mX)(mX)(mXëmX)(fX)(mX) | IS25 |
| | | (fX)(fX)(fX)(fX)(mX)(mX)(mX)(mX)(mX)(mX) | |
| | | (mX)(mX)(mX)*(dQ)*(SC6)(mX) | |

TABLE 51
Reduction of Target A

| | D15 | D15 | D15 | D15 | D15 |
|---|---|---|---|---|---|
| Compound | CBM | CTX | HPC | STR | CERV |
| 29 | 4 | 32 | 35 | 26 | 30 |
| 30 | 26 | 30 | 28 | 24 | 38 |

### Example 127: Effect of Compound 31 Targeting Target B in Various Brain Regions

Compound 31 was evaluated in an in vivo human Target B transgenic mice PD study. The animals received a single vehicle or 0.2 mg (10 mg/kg) dose via intracerebroventricular injection on day 1 (n=3/group). Animals were observed every day for behavioral changes. Brain regions were collected on day 15, and tissue was immediately placed in homogenizing tube, snap frozen, then kept at −80° C. for gene expression analysis.

RNA Isolation and qPCR was performed as described in Example 103, with the exceptions that instead of rat CTNNB1 TaqMan Gene Expression Assay, the human Target B TaqMan Gene Expression Assay (Thermo Fisher) was used; instead of rat ACTB (VIC) TaqMan Gene Expression Assay, the mouse GAPDH TaqMan Gene Expression Assay (Thermo Fisher: Mm99999915_g1, VIC) was used. Results are presented in the Table below as percent inhibition of Target B, relative to vehicle control.

TABLE 52

| | | | Ref ID |
|---|---|---|---|
| Compound | Backbone | Modified Strands (5′-3′) | NO: |
| 31 | fUëmA | (eX)*(fX)*(mX)(fX)(mX)(fX)(mX)(fX)(mX)(fX) | IA8 |
| | NB-198 | (mX)(fX)(mX)(fX)(mX)(fX)(mX)(fX)(mX)(fX)(mX)* | |
| | | (mX)*(mX) | |
| | | (AC6)*(mX)*(mX)(mX)(mX)(mX)(mX)(mX) | IS26 |
| | | (fXëmX)(fX)(fX)(fX)(fX)(mX)(mX)(mX)(mX)(mX) | |
| | | (mX)(mX)(mX)(mX)*(dQ)*(SC6)(mX) | |

TABLE 53
Reduction of Target B

| Compound | D15 STR | D15 MB | D15 CTX | D15 CERV |
|---|---|---|---|---|
| 31 | 8 | 57 | | 68 |

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize or be able to ascertain using no more than routine experimentation, many equivalents of the specific embodiments described herein. Such equivalents are intended to be encompassed by the following claims.

What is claimed is:

1. A compound of Formula (I′), or a salt thereof: 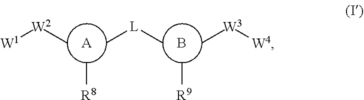
wherein:
A is a substituted tetrahydrofuranyl, substituted tetrahydropyranyl ring, or a modified sugar;
B is a substituted tetrahydrofuranyl, substituted tetrahydropyranyl ring, or a modified sugar;
W¹ and W⁴ are independently an modified or unmodified nucleoside, oligonucleotide, ligand, lipophilic moiety, or protecting group;
R⁸ and R⁹ are independently an substituted or unsubstituted heteroaryl;
L is a linker, a ligand, a lipophilic moiety, —Z¹—Y—Z²—, or a combination thereof;
W² and W³ are independently a bond, linker, substituted or unsubstituted alkylene, substituted or unsubstituted heteroalkylene, substituted or unsubstituted carbocyclylene, substituted or unsubstituted heterocyclylene, substituted or unsubstituted arylene, substituted or unsubstituted heteroarylene, —O—, —OP(O)O₂—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, S(O)₂NR^{A}—, —NR^{A}SO₂—, or a combination thereof;
each R^{A} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two R^{A} groups are joined to form with the atoms to which they are attached a substituted or unsubstituted heterocyclyl ring, or a substituted or unsubstituted heteroaryl ring;
Z¹ and Z² are independently a bond, substituted or unsubstituted alkylene, substituted or unsubstituted heteroalkylene, substituted or unsubstituted carbocyclylene, substituted or unsubstituted heterocyclylene, substituted or unsubstituted arylene, substituted or unsubstituted heteroarylene, —O—, —OP(O)O₂—, —N(R^{B})—, —S—, —C(═O)—, —C(═O)O—, —C(═O)NR^{B}—, —NR^{B}C(═O)—, —NR^{B}C(═O)R^{B}—, —C(═O)R^{B}—, —NR^{B}C(═O)O—, —NR^{B}C(═O)N(R^{B})—, —OC(O)—, —OC(═O)O—, —OC(═O)N(R^{B})—, S(O)₂NR^{B}—, —NR^{B}SO₂—, or a combination thereof;
each R^{B} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two R^{B} groups are joined to form a substituted or unsubstituted heterocyclyl ring, or a substituted or unsubstituted heteroaryl ring;
Y is substituted or unsubstituted alkylene, substituted or unsubstituted heteroalkylene, substituted or unsubstituted carbocyclylene, substituted or unsubstituted heterocyclylene, substituted or unsubstituted arylene, substituted or unsubstituted heteroarylene, —O—, —OP(O)O₂—, —N(R^{C})—, —S—, —C(═O)—, —C(═O)O—, —C(═O)NR^{C}—, —NR^{C}C(═O)—, —NR^{C}C(═O)R^{C}—, —C(═O)R^{C}—, —NR^{C}C(═O)O—, —NR^{C}C(═O)N(R^{C})—, —OC(═O)—, —OC(═O)O—, —OC(═O)N(R^{C})—, —S(O)₂NR^{C}—, —NR^{C}SO₂—, or a combination thereof; and
each R^{C} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group, or two R^{C} groups are joined to form a substituted or unsubstituted heterocyclyl ring, or a substituted or unsubstituted heteroaryl ring.

2. The compound of claim 1, or a salt thereof, which has Formula (VIII):

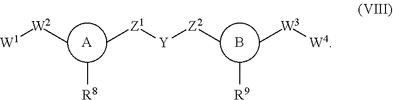

3. The compound of claim 1 or 2, wherein W¹ is a modified or unmodified nucleoside.

4. The compound of claim 1 or 2, wherein W¹ is an oligonucleotide.

5. The compound of claim 1 or 2, wherein W¹ is a protecting group.

6. The compound of any one of claims 1-5, wherein W⁴ is an modified or unmodified nucleoside.

7. The compound of any one of claims 1-5, wherein W⁴ is an oligonucleotide.

8. The compound of any one of claims 1-5, wherein W⁴ is a protecting group.

9. The compound of any one of claims 1-8, wherein W² is a bond.

10. The compound of any one of claims 1-8, wherein W² is a linker.

11. The compound of any one of claims 1-8, wherein W² is a substituted or unsubstituted heteroalkylene.

12. The compound of any one of claims 1-11, wherein W³ is a bond.

13. The compound of any one of claims 1-11, wherein W³ is a linker.

14. The compound of any one of claims 1-11, wherein W³ is a substituted or unsubstituted heteroalkylene.

15. The compound of any one of claims 1-14, wherein A is a substituted tetrahydrofuranyl.

16. The compound of any one of claims 1-15, wherein A is of the formula: 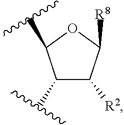
wherein
R² is —H, halogen, substituted or unsubstituted alkyl, substituted or unsubstituted alkenyl, substituted or unsubstituted alkynyl, —OR⁶, —N(R⁶), or —SR⁶;
R⁸ is an substituted or unsubstituted heteroaryl ring; and
each R⁶ is independently hydrogen, substituted or unsubstituted alkyl, or substituted or unsubstituted heteroalkyl.

17. The compound of any one of claims 1-14, wherein A is a substituted tetrahydropyranyl ring,

18. The compound of any one of claims 1-14, wherein A is a modified sugar.

19. The compound of any one of claims 1-15, wherein A is a bicyclic sugar.

20. The compound of claim 19, wherein the bicyclic sugar comprises a bridge between the 4′ and 2′ furanose ring atoms.

21. The compound of claim 19 or 20, wherein the 4′ to 2′ bridging substituents comprise 4′-(CH₂)₂-2′, 4′-(CH₂)₃-2′, 4′-CH₂—O-2′, or 4′-CH(CH₃)—O-2′.

22. The compound of any one of claims 1-15, wherein A is a sugar surrogate.

23. The compound of claim 22, wherein the sugar surrogate comprises a tetrahydropyran (THP), or a morpholino in place of the pentofuranosyl sugar.

24. The compound of claim 22, wherein the sugar surrogate is an unlocked nucleic acid.

25. The compound of any one of claims 1-24, wherein B is a substituted tetrahydrofuranyl.

26. The compound of any one of claims 1-25, wherein B is of the formula: 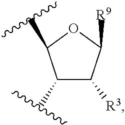
wherein:
R³ is —H, halogen, substituted or unsubstituted alkyl, substituted or unsubstituted alkenyl, substituted or unsubstituted alkynyl, —OR⁷, —N(R⁷), or —SR⁷;
R⁹ is an substituted or unsubstituted heteroaryl ring; and
each R⁷ is independently hydrogen, substituted or unsubstituted alkyl, or substituted or unsubstituted heteroalkyl.

27. The compound of any one of claims 1-24, wherein B is a substituted tetrahydropyranyl ring.

28. The compound of any one of claims 1-24, wherein B is a modified sugar.

29. The compound of any one of claims 1-24, wherein B is a bicyclic sugar.

30. The compound of claim 29, wherein the bicyclic sugar comprises a bridge between the 4′ and 2′ furanose ring atoms.

31. The compound of claim 29 or 30, wherein the 4′ to 2′ bridging substituents comprise 4′-(CH₂)₂-2′, 4′-(CH₂)₃-2′, 4′-CH₂—O-2′, or 4′-CH(CH₃)—O-2′.

32. The compound of any one of claims 1-24, wherein B is a sugar surrogate.

33. The compound of claim 32, wherein the sugar surrogate comprises a tetrahydropyran (THP), or a morpholino in place of the pentofuranosyl sugar.

34. The compound of claim 32, wherein the sugar surrogate is an unlocked nucleic acid.

35. A compound of Formula (I): 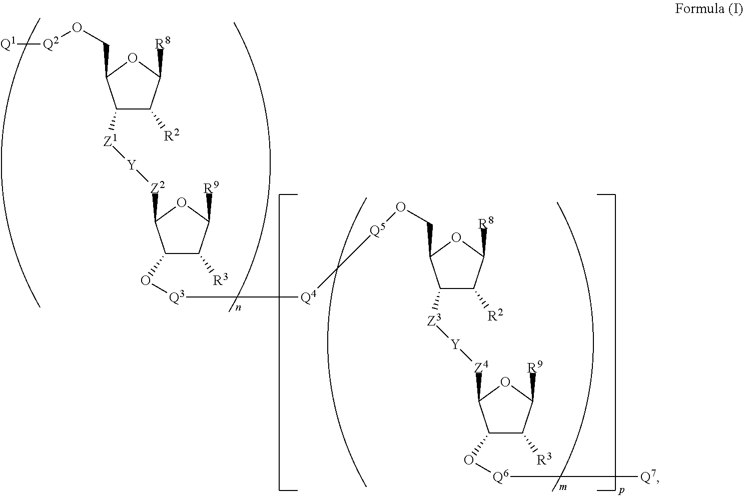
wherein:
Q¹ is —H, —OR⁴, a ligand, or a lipid;
each Q² is independently a bond, 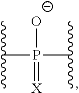
a ligand, a linker, or a lipid;
each Q³ is independently a bond, 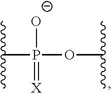
a ligand, a linker, or a lipid;
each Q⁴ is independently a bond, —R¹⁰O—, a ligand, a linker, or a lipid;
each Q⁵ is independently a bond, 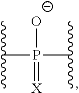
a ligand, a linker, or a lipid;
each Q⁶ is independently a bond, 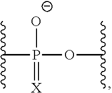
a ligand, a linker, or a lipid;
Q⁷ is —H, —R⁵, a ligand, or a lipid;
each Y is independently substituted or unsubstituted alkylene, substituted or unsubstituted heteroalkylene, substituted or unsubstituted carbocyclylene, substituted or unsubstituted heterocyclylene, substituted or unsubstituted arylene, substituted or unsubstituted heteroarylene, —O—, —OP(O)O₂—, —N(R^{C})—, —S—, —C(═O)—, —C(═O)O—, —C(═O)NR^{C}—, —NR^{C}C(═O)—, —NR^{C}C(═O)R^{C}—, —C(═O)R^{C}—, —NR^{C}C(═O)O—, —NR^{C}C(═O)N(R^{C})—, —OC(═O)—, —OC(═O)O—, —OC(═O)N(R^{C})—, —S(O)₂NR^{C}—, —NR^{C}SO₂—, or a combination thereof;
each R^{C} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group, or two R^{C} groups are joined to form a substituted or unsubstituted heterocyclyl ring, or a substituted or unsubstituted heteroaryl ring;
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently an substituted or unsubstituted heteroaryl ring;
each R⁹ is independently an substituted or unsubstituted heteroaryl ring;
each R¹⁰ is independently an oligonucleotide;
each X is independently O or S;
each instance of Z¹, Z², Z³, or Z⁴ is independently a bond, C₁-C₆ alkylene, or C₂-C₆ alkenylene;
n is 1, 2, 3, 4, 5, 6, 7, 8, 9, or 10;
each m is independently 1, 2, 3, 4, 5, 6, 7, 8, 9, or 10; and
p is 0, 1, 2, 3, 4, 5, 6, 7, 8, 9, or 10;
or a salt or prodrug thereof.

36. The compound of claim 35, wherein Q³ is

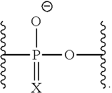

37. The compound of claim 35 or 36, wherein Q⁴ is —R¹⁰O—.

38. The compound of any one of claims 35-37, wherein Q⁵ is

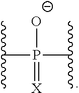

39. The compound of any one of claim 35-38, wherein Q⁶ is

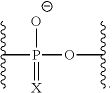

40. The compound of any one of claims 35-39, wherein Q⁷ is —R⁵.

41. A compound of Formula (VII): 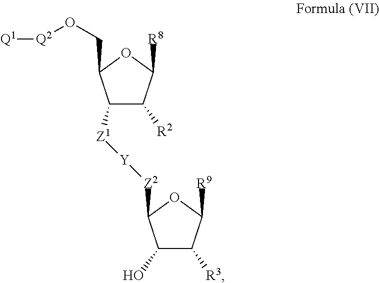
wherein:
Y is —C(═O)N(R^{C})—, or —N(R^{C})C(═O)—;
Q¹ is —H, —OR⁴, a ligand, a linker, or a lipid;
Q² is independently a bond, 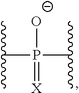
a ligand, a linker, or a lipid;
R^{C} is independently —H, substituted or unsubstituted alkyl, substituted or unsubstituted alkenyl, substituted or unsubstituted alkynyl, substituted or unsubstituted heteroalkyl, substituted or unsubstituted aryl, or substituted or unsubstituted heteroaryl;
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently an substituted or unsubstituted heteroaryl;
each R⁹ is independently an substituted or unsubstituted heteroaryl;
each instance of Z¹ or Z² is independently a bond, C₁-C₆ alkylene, or C₂-C₆ alkenylene; and
each X is independently O or S;
or a salt thereof.

42. The compound of any one of claims 35-41, wherein Q¹ is —OR⁴.

43. The compound of any one of claims 35-42, wherein Q² is

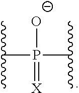

44. A compound of Formula (II): 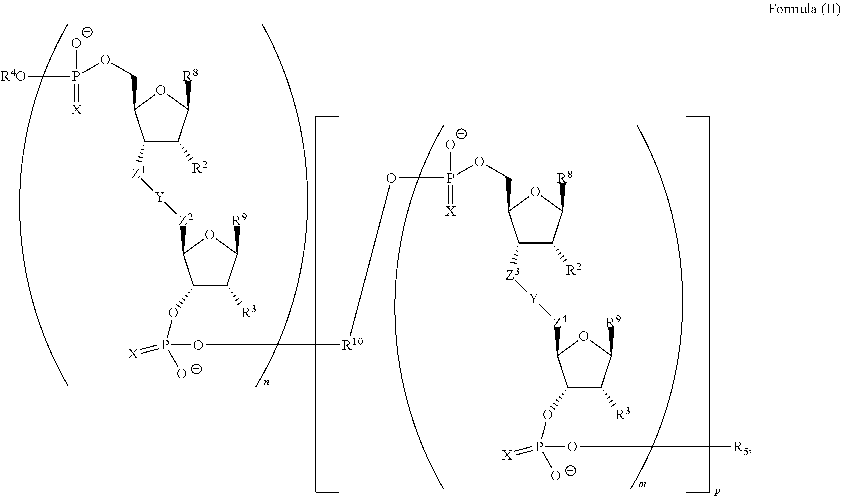
wherein:
each Y is independently substituted or unsubstituted alkylene, substituted or unsubstituted heteroalkylene, substituted or unsubstituted carbocyclylene, substituted or unsubstituted heterocyclylene, substituted or unsubstituted arylene, substituted or unsubstituted heteroarylene, —O—, —OP(O)O₂—, —N(R^{C})—, —S—, —C(═O)—, —C(═O)O—, —C(═O)NR^{C}—, —NR^{C}C(═O)—, —NR^{C}C(═O)R^{C}—, —C(═O)R^{C}—, —NR^{C}C(═O)O—, —NR^{C}C(═O)N(R^{C})—, —OC(═O)—, —OC(═O)O—, —OC(═O)N(R^{C})—, —S(O)₂NR^{C}—, —NR^{C}SO₂—, or a combination thereof;
each R^{C} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group, or two R^{C} groups are joined to form a substituted or unsubstituted heterocyclyl ring, or a substituted or unsubstituted heteroaryl ring;
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl;
each R⁹ is independently a substituted or unsubstituted heteroaryl;
each R¹⁰ is independently an oligonucleotide;
each X is independently O or S;
each instance of Z¹, Z², Z³, or Z⁴ is independently a bond, C₁-C₆ alkylene, or C₂-C₆ alkenylene;
n is 1, 2, 3, 4, 5, 6, 7, 8, 9, or 10;
each m is independently 1, 2, 3, 4, 5, 6, 7, 8, 9, or 10; and
p is 0, 1, 2, 3, 4, 5, 6, 7, 8, 9, or 10;
or a salt or prodrug thereof.

45. The compound of any one of claims 35-40, or 42-44, wherein p is 0.

46. The compound of any one of claims 35-40, or 42-44, wherein p is 1, 2, or 3.

47. The compound of claim 44 or 45, wherein the compound is of the Formula (II-a): 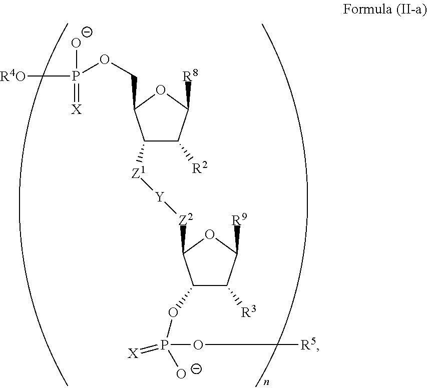
wherein:
each Y is independently substituted or unsubstituted alkylene, substituted or unsubstituted heteroalkylene, substituted or unsubstituted carbocyclylene, substituted or unsubstituted heterocyclylene, substituted or unsubstituted arylene, substituted or unsubstituted heteroarylene, —O—, —OP(O)O₂—, —N(R^{C})—, —S—, —C(═O)—, —C(═O)O—, —C(═O)NR^{C}—, —NR^{C}C(═O)—, —NR^{C}C(═O)R^{C}—, —C(═O)R^{C}—, —NR^{C}C(═O)O—, —NR^{C}C(═O)N(R^{C})—, —OC(═O)—, —OC(═O)O—, —OC(═O)N(R^{C})—, —S(O)₂NR^{C}—, —NR^{C}SO₂—, or a combination thereof;
each R^{C} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group, or two R^{C} groups are joined to form a substituted or unsubstituted heterocyclyl ring, or a substituted or unsubstituted heteroaryl ring;
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently an substituted or unsubstituted heteroaryl;
each R⁹ is independently an substituted or unsubstituted heteroaryl;
each X is independently O or S;
each instance of Z¹, and Z², is independently a bond, C₁-C₆ alkylene, or C₂-C₆ alkenylene; and
n is 1, 2, 3, 4, 5, 6, 7, 8, 9, or 10;
or a salt thereof.

48. A compound of Formula (VI): 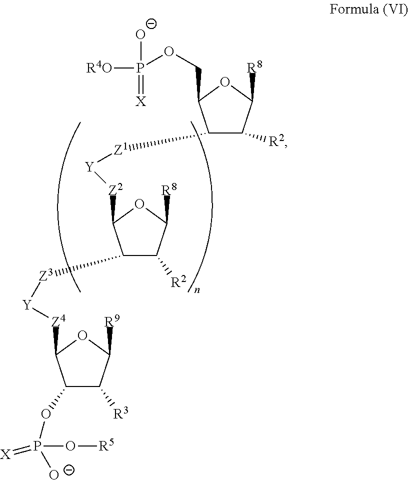
wherein:
each Y is independently substituted or unsubstituted alkylene, substituted or unsubstituted heteroalkylene, substituted or unsubstituted carbocyclylene, substituted or unsubstituted heterocyclylene, substituted or unsubstituted arylene, substituted or unsubstituted heteroarylene, —O—, —OP(O)O₂—, —N(R^{C})—, —S—, —C(═O)—, —C(═O)O—, —C(═O)NR^{C}—, —NR^{C}C(═O)—, —NR^{C}C(═O)R^{C}—, —C(═O)R^{C}—, —NR^{C}C(═O)O—, —NR^{C}C(═O)N(R^{C})—, —OC(═O)—, —OC(═O)O—, —OC(═O)N(R^{C})—, —S(O)₂NR^{C}—, —NR^{C}SO₂—, or a combination thereof;
each R^{C} is independently hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group, or two R^{C} groups are joined to form a substituted or unsubstituted heterocyclyl ring, or a substituted or unsubstituted heteroaryl ring;
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are each independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently an substituted or unsubstituted heteroaryl;
each R⁹ is independently an substituted or unsubstituted heteroaryl;
each X is independently O or S;
each instance of Z¹, Z², Z³, or Z⁴ is independently a bond, C₁-C₆ alkylene, or C₂-C₆ alkenylene;
n is 1, 2, 3, 4, 5, 6, 7, 8, 9, or 10;
or a salt or prodrug thereof.

49. The compound of any one of claims 35-40, 42-46, or 48, wherein at least one instance of Z³ is C₁-C₆ alkylene.

50. The compound of claim 35-40, 42-46, or 48-49, wherein Z³ is —CH₂— or —CH₂CH₂—.

51. The compound of any one of claims 35-40, 42-46, or 48, wherein at least one instance of Z³ is C₂-C₆ alkenylene.

52. The compound of claim 35-40, 42-46, or 48, wherein Z³ is —CH═CH—.

53. The compound of any one of claims 35-40, 42-46, or 48, wherein at least one instance of Z³ is a bond.

54. The compound of any one of claims 35-40, 42-46, or 48-53, wherein at least one instance of Z⁴ is C₁-C₆ alkylene.

55. The compound of any one of claims 35-40, 42-46, or 48-54, wherein Z⁴ is —CH₂— or —CH₂CH₂—.

56. The compound of any one of claims 35-40, 42-46, or 48-53, wherein at least one instance of Z⁴ is C₂-C₆ alkenylene.

57. The compound of any one of claims 35-40, 42-46, 48-53, or 56, wherein Z⁴ is —CH═CH—.

58. The compound of any one of claims 35-40, 42-46, or 48-53, wherein at least one instance of Z⁴ is a bond.

59. The compound of any one of claims 35-40, or 42-58, wherein n is 1.

60. The compound of any one of claims 35-40, or 42-58, wherein n is 2.

61. The compound of any one of claims 35-40, or 42-58, wherein n is 3.

62. The compound of any one of claims 1-61, wherein at least one instance of Z¹ is C₁-C₆ alkylene.

63. The compound of any one of claims 1-62, wherein Z¹ is —CH₂— or —CH₂CH₂—.

64. The compound of any one of claims 1-61, wherein at least one instance of Z¹ is C₂-C₆ alkenylene.

65. The compound of any one of claims 1-61, or 64, wherein Z¹ is —C(H)═C(H)—.

66. The compound of any one of claims 1-61, wherein at least one instance of Z¹ is a bond.

67. The compound of any one of claims 1-66, wherein at least one instance of Z² is C₁-C₆ alkylene.

68. The compound of any one of claims 1-67, wherein Z² is —CH₂— or —CH₂CH₂—.

69. The compound of any one of claims 1-66, wherein at least one instance of Z² is C₂-C₆ alkenylene.

70. The compound of any one of claims 1-66, or 69, wherein Z² is —C(H)═C(H)—.

71. The compound of any one of claims 1-66, wherein at least one instance of Z² is a bond.

72. The compound of any one of claims 1-71, wherein each Y is independently —C(═O)N(R^{C})— or N(R^{C})C(═O)—.

73. The compound of any one of claims 1-71, wherein each Y is independently comprises at least one lipophilic moiety.

74. An oligonucleotide of Formula (IX): 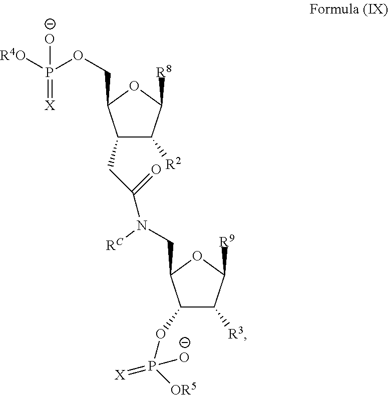
wherein:
R^{C} is —H, substituted or unsubstituted alkyl, substituted or unsubstituted alkenyl, substituted or unsubstituted alkynyl, substituted or unsubstituted heteroalkyl, substituted or unsubstituted aryl, or substituted or unsubstituted heteroaryl;
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

75. An oligonucleotide of Formula (X): 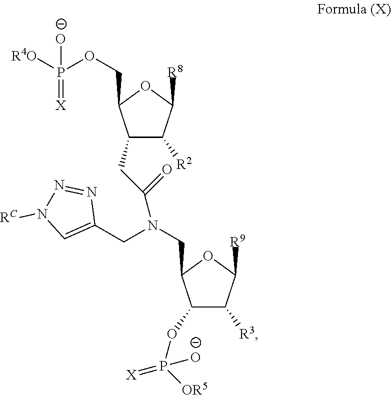
wherein:
R^{C} is —H, substituted or unsubstituted alkyl, substituted or unsubstituted alkenyl, substituted or unsubstituted alkynyl, substituted or unsubstituted heteroalkyl, substituted or unsubstituted aryl, or substituted or unsubstituted heteroaryl;
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

76. An oligonucleotide of Formula (XI): 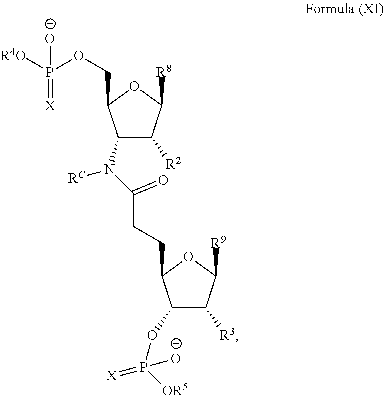
wherein:
R^{C} is —H, substituted or unsubstituted alkyl, substituted or unsubstituted alkenyl, substituted or unsubstituted alkynyl, substituted or unsubstituted heteroalkyl, substituted or unsubstituted aryl, or substituted or unsubstituted heteroaryl;
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

77. An oligonucleotide of Formula (XII): 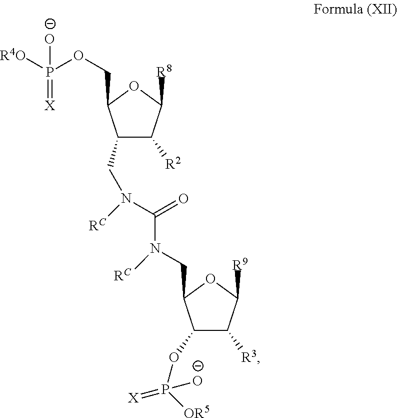
wherein:
each instance of R^{C} is independently —H, substituted or unsubstituted alkyl, substituted or unsubstituted alkenyl, substituted or unsubstituted alkynyl, substituted or unsubstituted heteroalkyl, substituted or unsubstituted aryl, or substituted or unsubstituted heteroaryl;
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

78. An oligonucleotide of Formula (XIII): 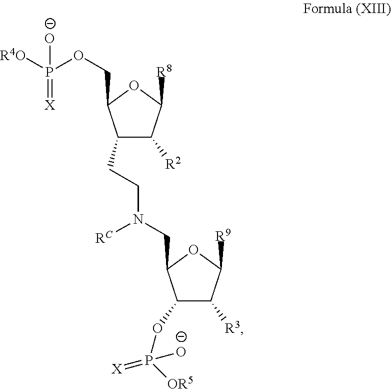
wherein:
R^{C} is —H, substituted or unsubstituted alkyl, substituted or unsubstituted alkenyl, substituted or unsubstituted alkynyl, substituted or unsubstituted heteroalkyl, substituted or unsubstituted aryl, or substituted or unsubstituted heteroaryl;
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

79. An oligonucleotide of Formula (XIV): 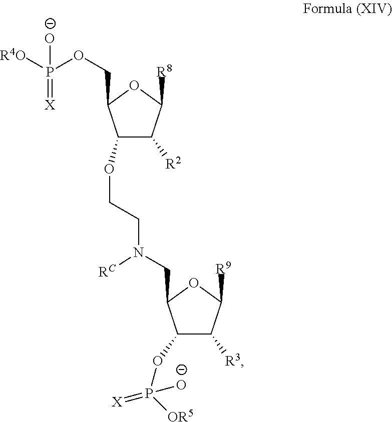
wherein:
R^{C} is —H, substituted or unsubstituted alkyl, substituted or unsubstituted alkenyl, substituted or unsubstituted alkynyl, substituted or unsubstituted heteroalkyl, substituted or unsubstituted aryl, or substituted or unsubstituted heteroaryl;
each R² is independently —H, halogen, substituted or unsubstituted alkyl, substituted or unsubstituted alkenyl, substituted or unsubstituted alkynyl, —OR⁶, —N(R⁶), or —SR₆;
each R³ is independently —H, halogen, substituted or unsubstituted alkyl, substituted or unsubstituted alkenyl, substituted or unsubstituted alkynyl, —OR⁷, —N(R⁷), or —SR₇;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

80. The compound of any one of claims 1-79, wherein each instance of R^{C} is independently substituted or unsubstituted alkyl.

81. The compound of any one of claims 1-80, wherein each instance of R^{C} is independently substituted or unsubstituted —C₈-C₁₀₀-alkyl.

82. The compound of any one of claims 1-81, wherein each instance of R^{C} is independently substituted or unsubstituted —C₈-C₄₀-alkyl.

83. The compound of any one of claims 1-82, wherein each instance of R^{C} is independently substituted or unsubstituted —C₈-C₂₀-alkyl.

84. The compound of any one of claims 1-83, wherein each instance of R^{C} is independently substituted or unsubstituted —C₁₂-C₂₀-alkyl.

85. The compound of any one of claims 1-83, wherein each instance of R^{C} is independently substituted or unsubstituted —C₁₆-C₂₀-alkyl.

86. The compound of any one of claims 1-80, wherein each instance of R^{C} is independently substituted or unsubstituted —C₁-C₃₀-alkyl.

87. The compound of any one of claims 1-80, wherein each instance of R^{C} is independently substituted or unsubstituted —C₁-C₂₀-alkyl.

88. The compound of any one of claims 1-80, wherein each instance of R^{C} is independently substituted or unsubstituted —C₁₀-C₂₀-alkyl.

89. The compound of any one of claims 1-88, wherein R^{c} is substituted with one or more of a hydroxyl, amine, carboxylic acid, azide, or alkyne.

90. The compound of any one of claims 1-88, wherein Rc is substituted with a carboxylic acid.

91. The compound of any one of claims 1-88, wherein Rc comprises at least one substituent selected from the group consisting of hexanoic acid, heptanoic acid, octanoic acid, nonanoic acid, decanoic acid, undecanoic acid, dodcanoic acid, tridecanoic acid, tetradecanoic acid, pentadecanoic acid, hexadecanoic acid, heptadecanoic acid, octadecanoic acid, oleic acid, linoleic acid, and arachidonic acid.

92. The compound of any one of claims 1-79, wherein each instance of R^{C} is independently —H.

93. An oligonucleotide of Formula (IX-a): 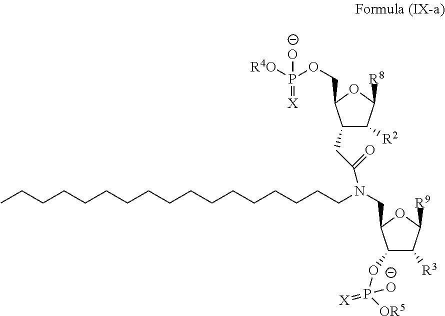
wherein:
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

94. An oligonucleotide of Formula (IX-b): 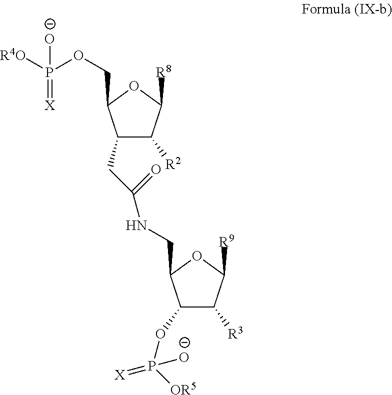
wherein:
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

95. An oligonucleotide of Formula (X-a): 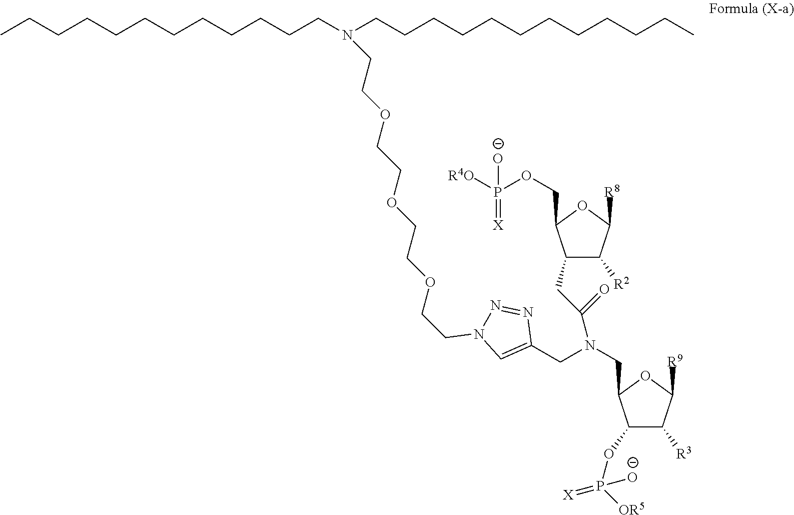
wherein:
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

96. An oligonucleotide of Formula (XV): 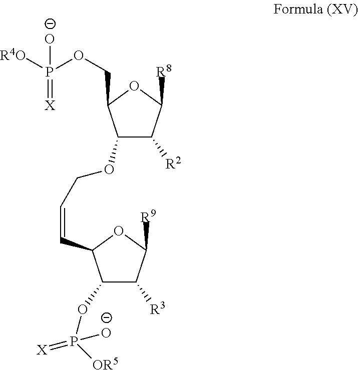
wherein:
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

97. An oligonucleotide of Formula (XVI): 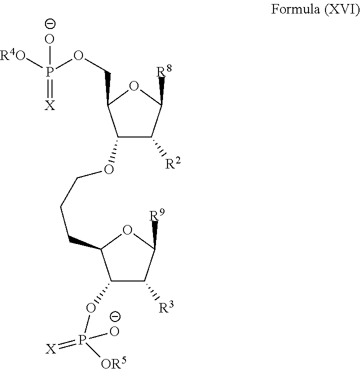
wherein:
each R² is independently —H, halogen, substituted or unsubstituted alkyl, substituted or unsubstituted alkenyl, substituted or unsubstituted alkynyl, —OR₆, —N(R⁶), or —SR₆;
each R³ is independently —H, halogen, substituted or unsubstituted alkyl, substituted or unsubstituted alkenyl, substituted or unsubstituted alkynyl, —OR₇, —N(R⁷), or —SR₇;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

98. An oligonucleotide of Formula (XVII): 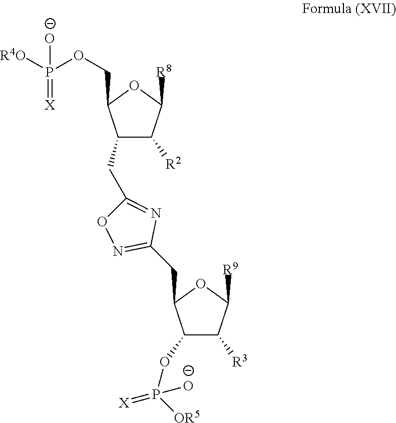
wherein:
each R² is independently —H, halogen, substituted or unsubstituted alkyl, substituted or unsubstituted alkenyl, substituted or unsubstituted alkynyl, —OR⁶, —N(R⁶), or —SR⁶;
each R³ is independently —H, halogen, substituted or unsubstituted alkyl, substituted or unsubstituted alkenyl, substituted or unsubstituted alkynyl, —OR⁷, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

99. An oligonucleotide of Formula (XVIII): 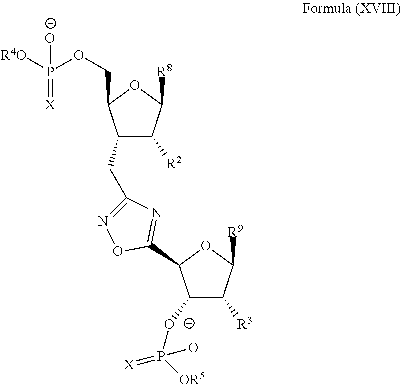
wherein:
each R² is independently —H, halogen, substituted or unsubstituted alkyl, substituted or unsubstituted alkenyl, substituted or unsubstituted alkynyl, —OR₆, —N(R⁶), or —SR⁶;
each R³ is independently —H, halogen, substituted or unsubstituted alkyl, substituted or unsubstituted alkenyl, substituted or unsubstituted alkynyl, —OR₇, —N(R⁷), or —SR⁷;
R⁴ and R⁵ are independently an oligonucleotide, a protecting group, or R⁴ and R⁵ are joined together to form a single oligonucleotide;
each R⁶ is independently hydrogen, substituted or unsubstituted alkyl, or substituted or unsubstituted heteroalkyl;
each R⁷ is independently hydrogen, substituted or unsubstituted alkyl, or substituted or unsubstituted heteroalkyl;
each R⁸ is independently a substituted or unsubstituted heteroaryl ring;
each R⁹ is independently a substituted or unsubstituted heteroaryl ring; and
each X is independently O or S;
or a salt or prodrug thereof.

100. The compound of any one of claims 16-99, wherein at least one instance of R² is —OR⁶.

101. The compound of any one of claims 16-100, wherein at least one instance of R⁶ is —C₁-C₆-alkyl.

102. The compound of any one of claims 16-101, wherein at least one instance of R⁶ is methyl.

103. The compound of any one of claims 16-99, wherein at least one instance of R² is —F.

104. The compound of any one of claims 26-103, wherein at least one instance of R³ is —OR⁷.

105. The compound of any one of claims 26-104, wherein at least one instance of R⁷ is —C₁-C₆-alkyl.

106. The compound of any one of claims 26-105, wherein at least one instance of R⁷ is methyl.

107. The compound of any one of claims 26-103, wherein at least one instance of R³ is —F.

108. The compound of any one of claims 1-107, wherein at least one instance of R⁸ is a nucleobase.

109. The compound of any one of claims 1-108, wherein at least one instance of R⁸ is uracil, cytosine, adenine, guanine, inosine, or thymine.

110. The compound of any one of claims 1-108, wherein at least one instance of R⁸ is a modified nucleobase.

111. The compound of any one of claims 1-109, wherein at least one instance of R⁸ is uracil.

112. The compound of any one of claims 1-109, wherein at least one instance of R⁸ is cytosine.

113. The compound of any one of claims 1-112, wherein at least one instance of R⁹ is a nucleobase.

114. The compound of any one of claims 1-113, wherein at least one instance of R⁹ is uracil, cytosine, adenine, guanine, inosine, or thymine.

115. The compound of any one of claims 1-113, wherein at least one instance of R⁹ is a modified nucleobase.

116. The compound of any one of claims 1-113, wherein at least one instance of R⁹ is uracil.

117. The compound of any one of claims 1-113, wherein at least one instance of R⁹ is cytosine.

118. An oligonucleotide of Formula (IX-c): 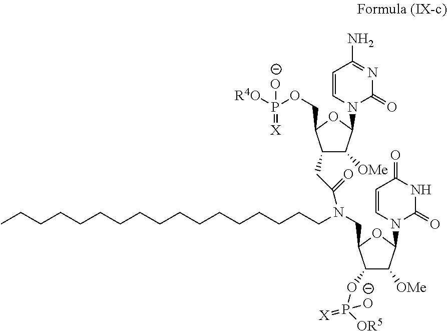
wherein:
R⁴ and R⁵ are independently an oligonucleotide, a protecting group, or R⁴ and R⁵ are joined together to form a single oligonucleotide; and
each X is independently O or S;
or a salt or prodrug thereof.

119. An oligonucleotide of Formula (IX-d): 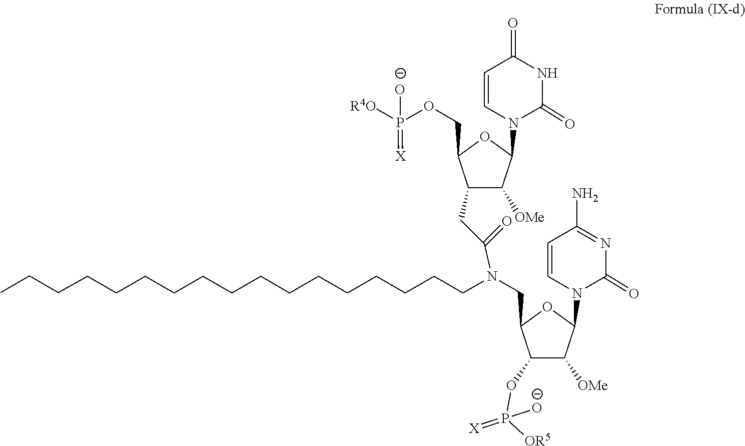
wherein:
R⁴ and R⁵ are independently an oligonucleotide, a protecting group, or R⁴ and R⁵ are joined together to form a single oligonucleotide; and
each X is independently O or S;
or a salt or prodrug thereof.

120. An oligonucleotide of Formula (IX-e): 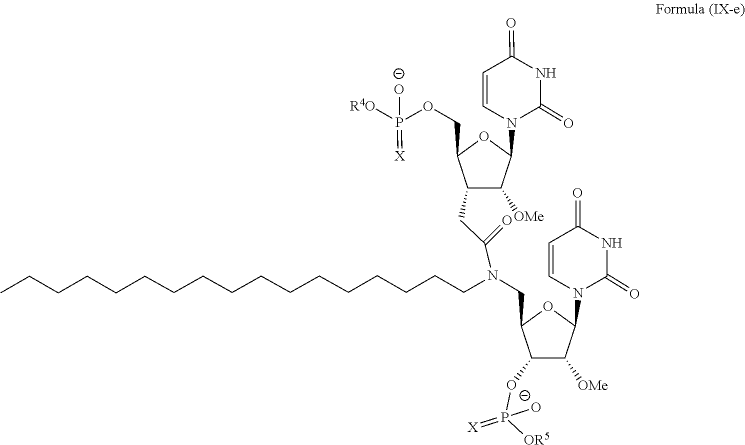
wherein:
R⁴ and R⁵ are independently an oligonucleotide, a protecting group, or R⁴ and R⁵ are joined together to form a single oligonucleotide; and
each X is independently O or S;
or a salt or prodrug thereof.

121. The compound of any one of claims 1-120, wherein R⁴ and R⁵ are each independently an oligonucleotide.

122. The compound of any one of claims 1-120, wherein R⁴ is an oligonucleotide; and R⁵ is a protecting group.

123. The compound of any one of claims 1-120, wherein R⁴ is a protecting group; and R⁵ is an oligonucleotide.

124. The compound of any one of claims 1-120, wherein R⁴ and R⁵ are each independently a protecting group.

125. The compound of any one of claims 1-122, wherein R⁴ comprises an oligonucleotide attached at its 5′ end.

126. The compound of any one of claims 1-122, wherein R⁴ comprises an oligonucleotide attached at its 3′ end.

127. The compound of any one of claims 1-122, wherein R⁴ comprises an oligonucleotide attached at an internal position on the oligonucleotide.

128. The compound of any one of claims 1-121, 123, 125-127 wherein R⁵ comprises an oligonucleotide attached at its 5′ end.

129. The compound of any one of claims 1-121, 123, 125-127 wherein R⁵ comprises an oligonucleotide attached at its 3′ end.

130. The compound of any one of claims 1-121, 123, 125-127, wherein R⁵ comprises an oligonucleotide attached at an internal position on the oligonucleotide.

131. The compound of any one of claims 1-130, wherein the protecting group, if present, is an oxygen protecting group.

132. The compound of any one of claims 1-131, wherein the protecting group is acid-labile oxygen protecting group.

133. The compound of any one of claims 1-132, wherein the protecting group is 4,4′-dimethoxytrityl or 2-cyanoethyl 5′-O-(4,4′-dimethoxytrityl)thymidine-3′-O—(N,N-diisopropylamino)phosphoramidite.

134. The compound of any one of claims 35-133, wherein at least one X is O.

135. The compound of any one of claims 35-134, wherein at least two instances of X are O.

136. The compound of any one of claims 35-134, wherein at least one X is S.

137. The compound of any one of claims 35-133, or 136 wherein at least two instances of X are S.

138. The compound of any one of claims 35-134, wherein at least one instance of X is O, and at least one instance of X is S.

139. The compound of any one of claims 1-138, wherein the oligonucleotide comprises at least one siRNA, an miRNA, an ADAR recruiting molecule, an ADAR targeting molecule, a guide RNA, and/or an antisense nucleic acid.

140. The compound of any one of claims 1-139, wherein the compound comprises at least one oligonucleotide.

141. A compound of the formula: 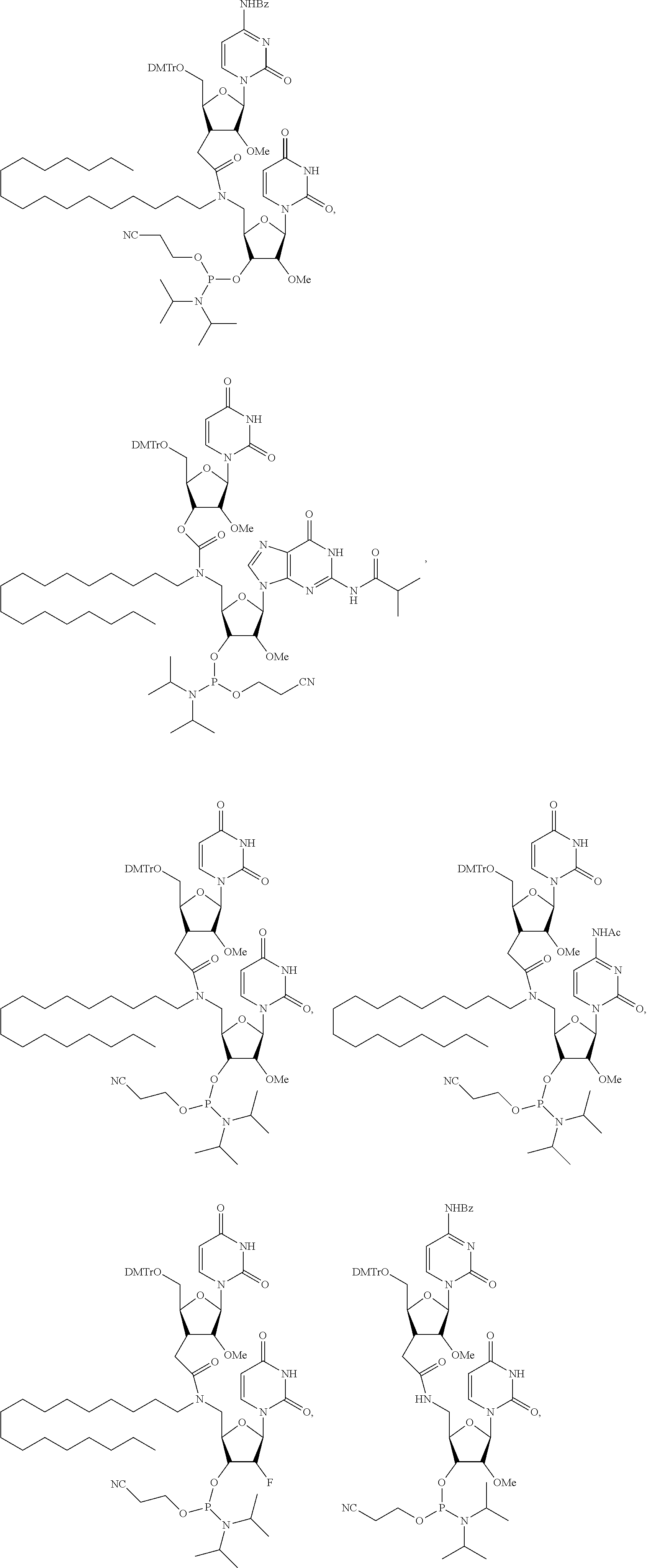 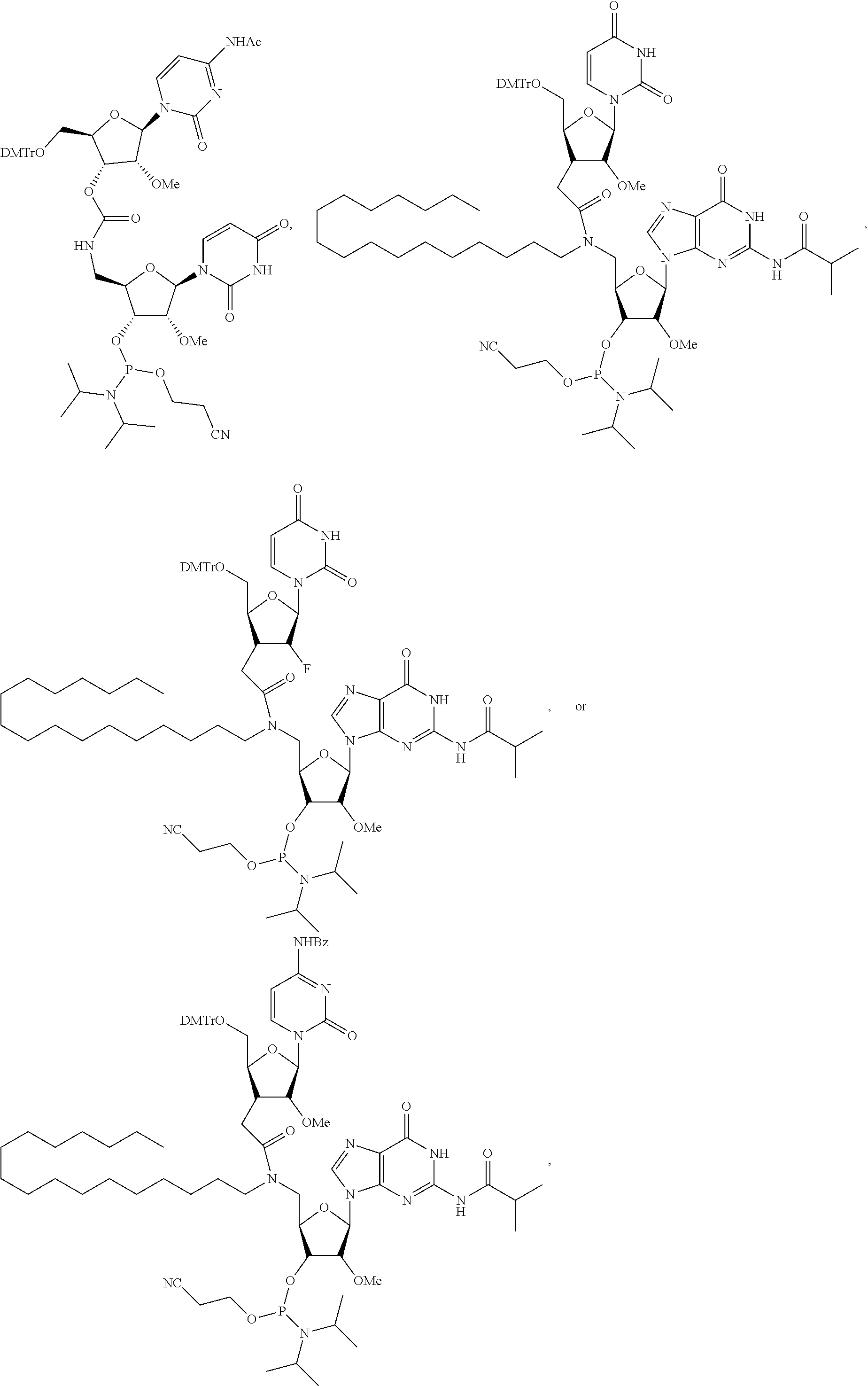
or a salt thereof.

142. A compound of the formula 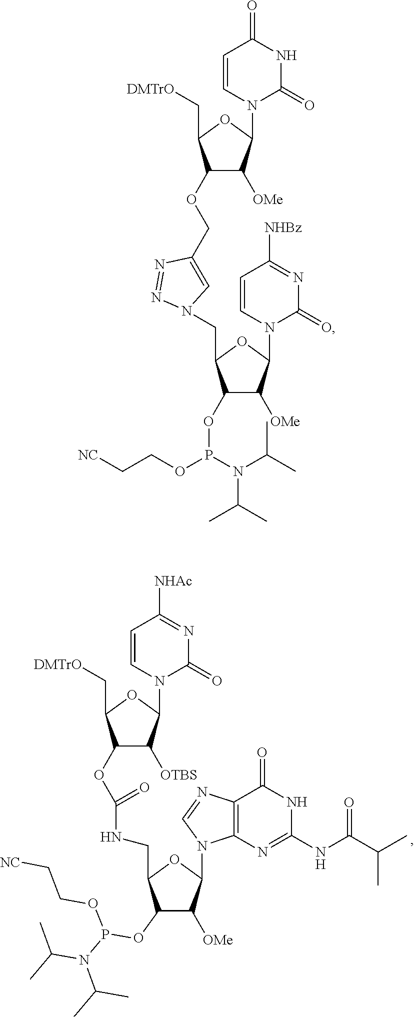 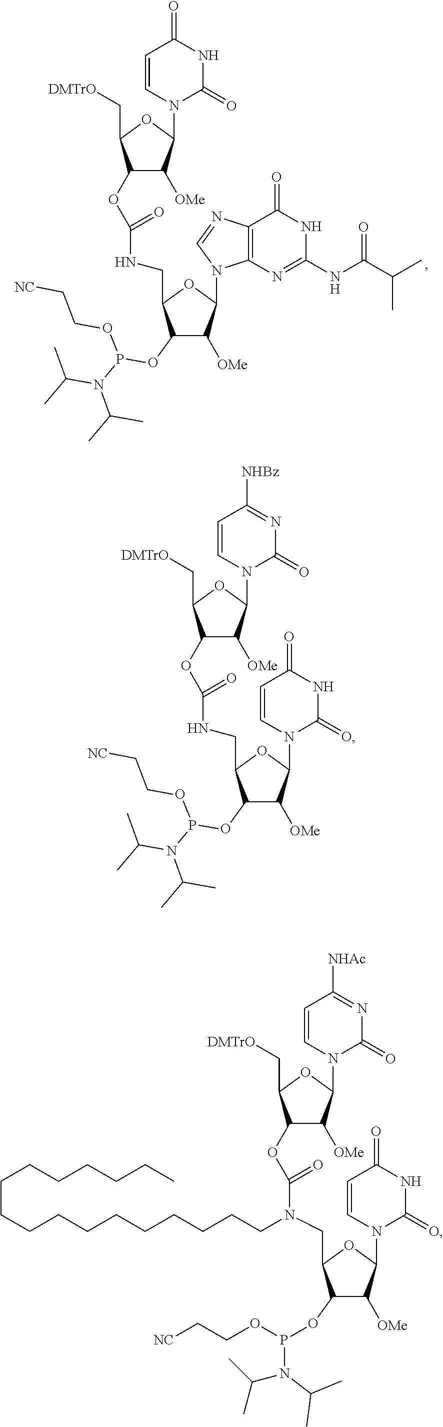 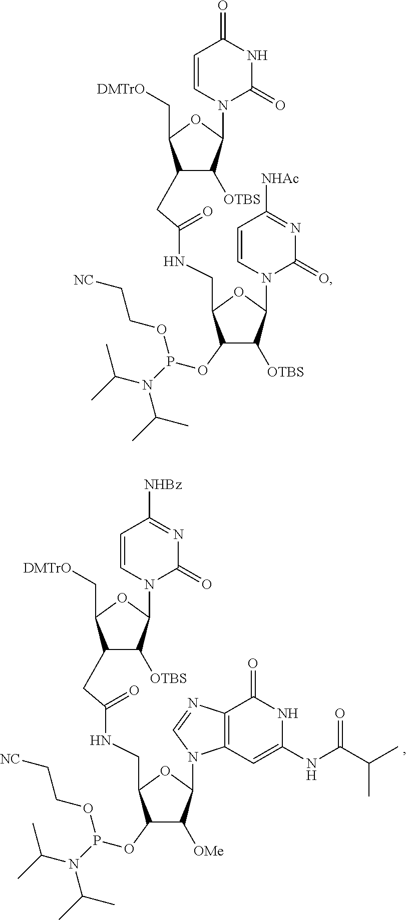 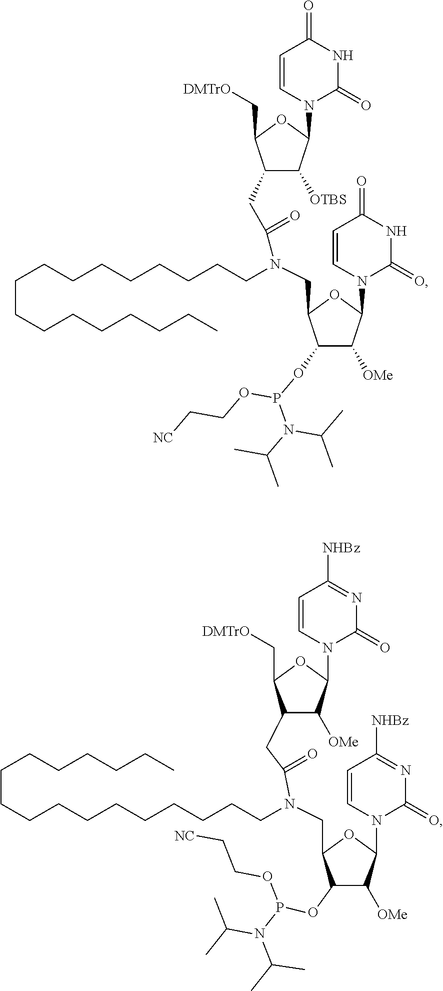 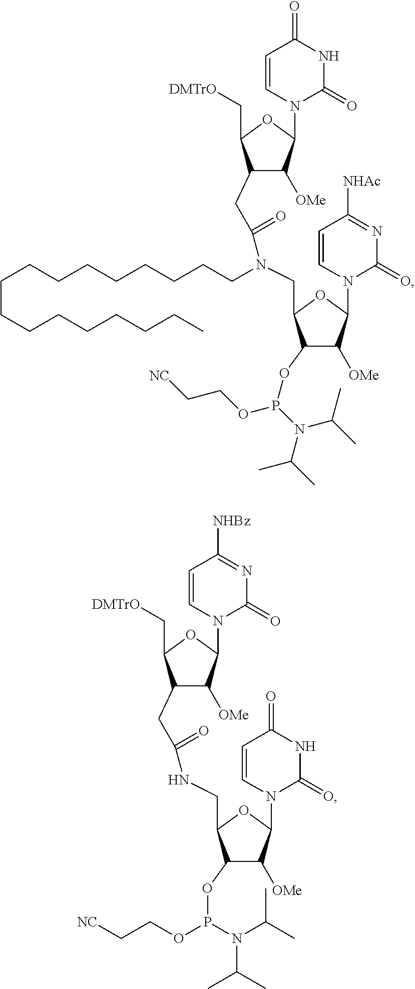 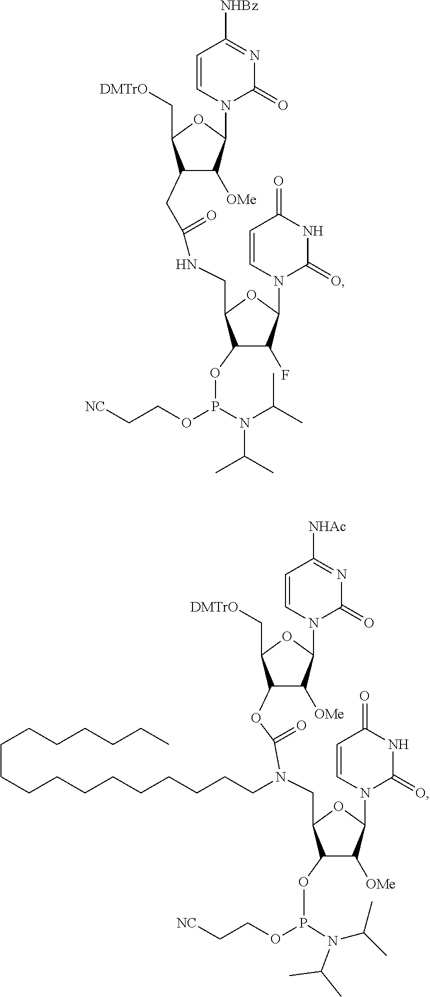 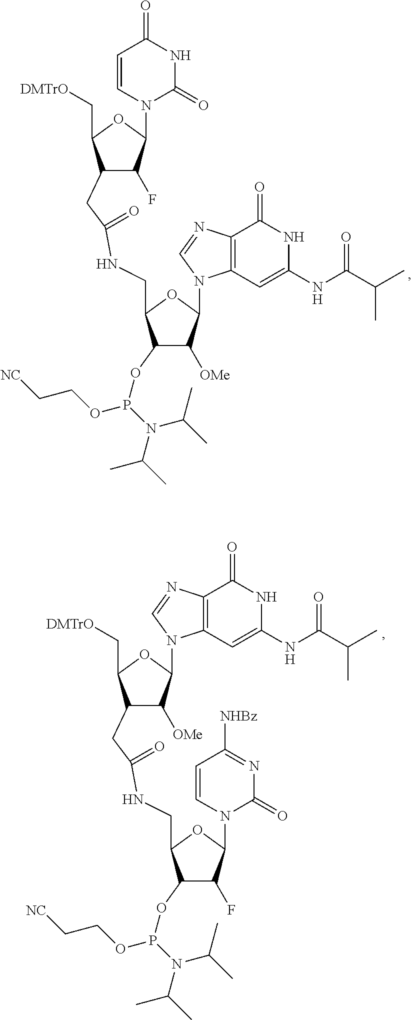 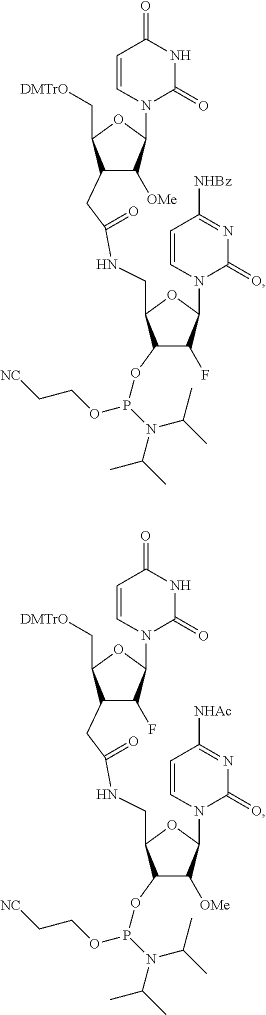 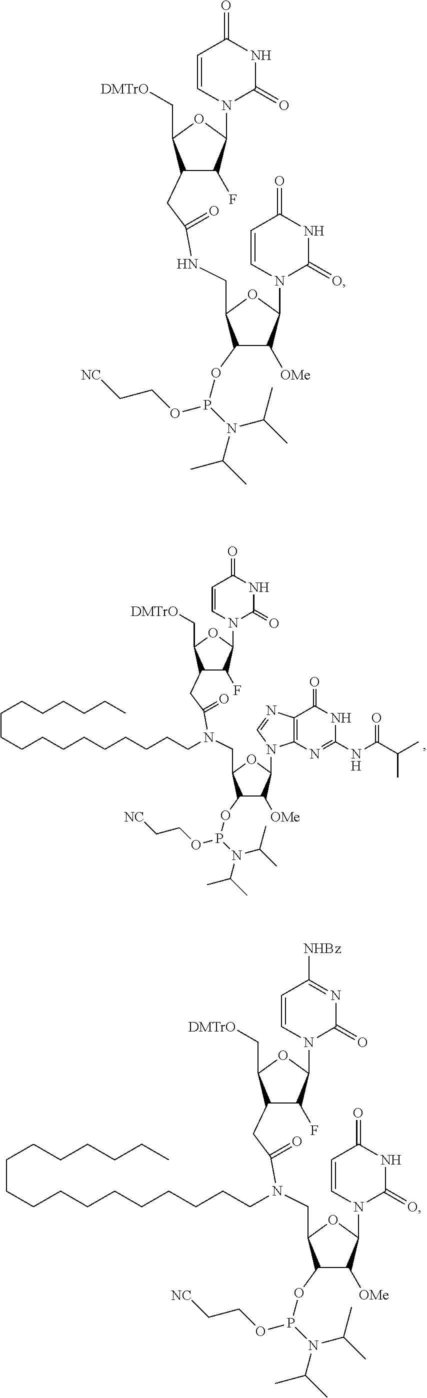 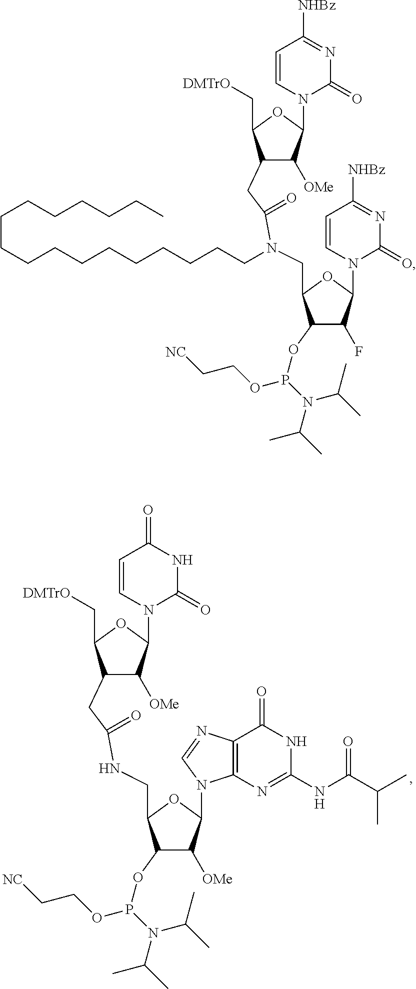 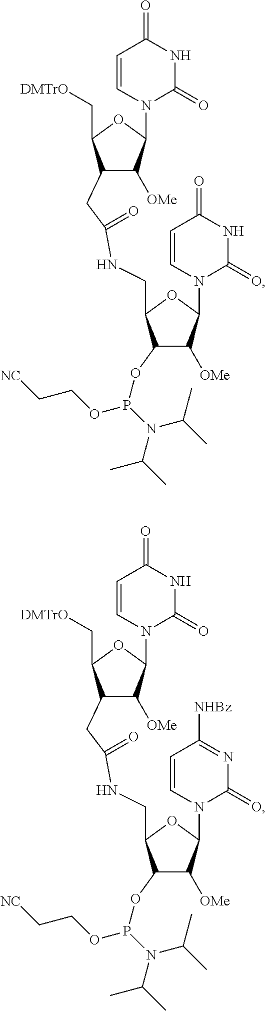 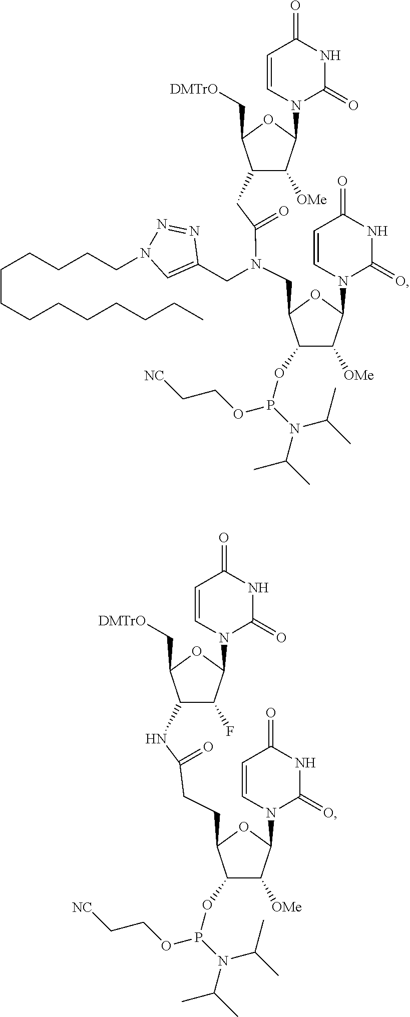 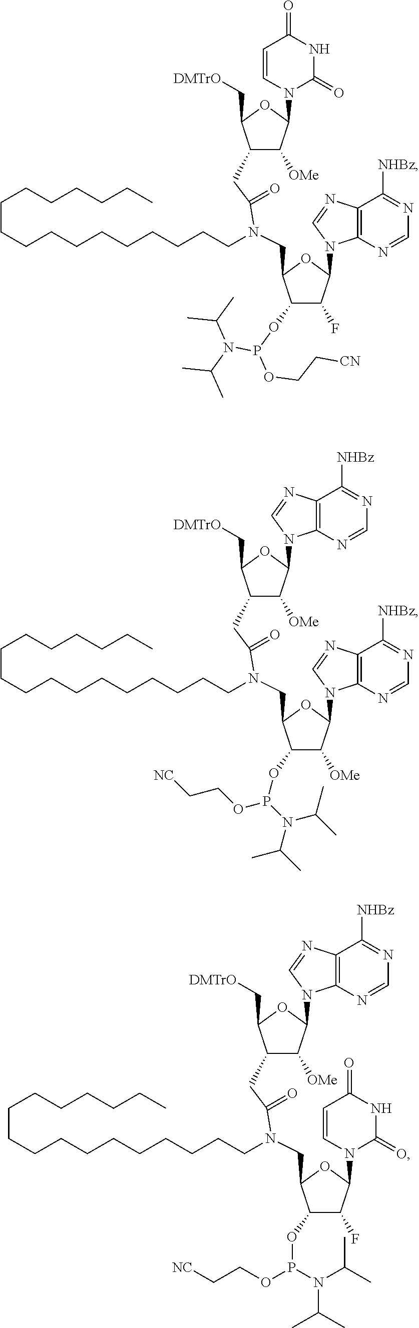 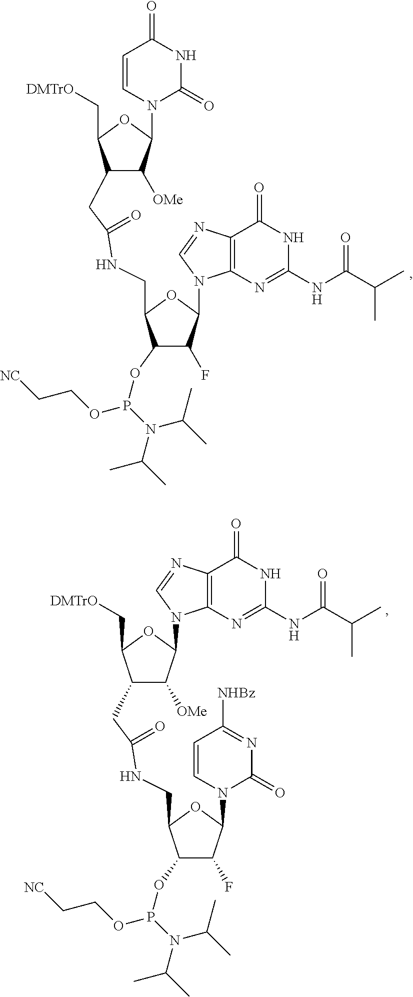 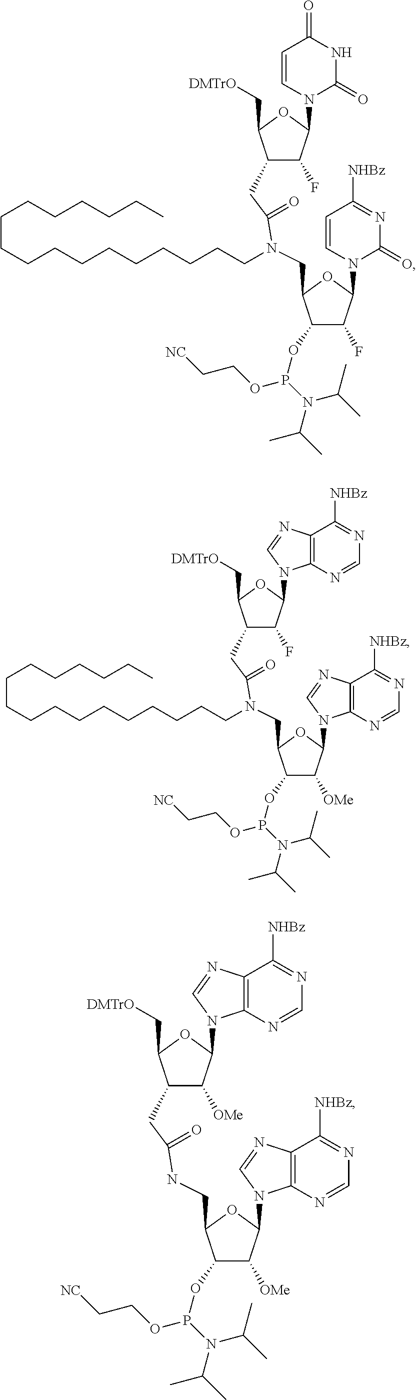 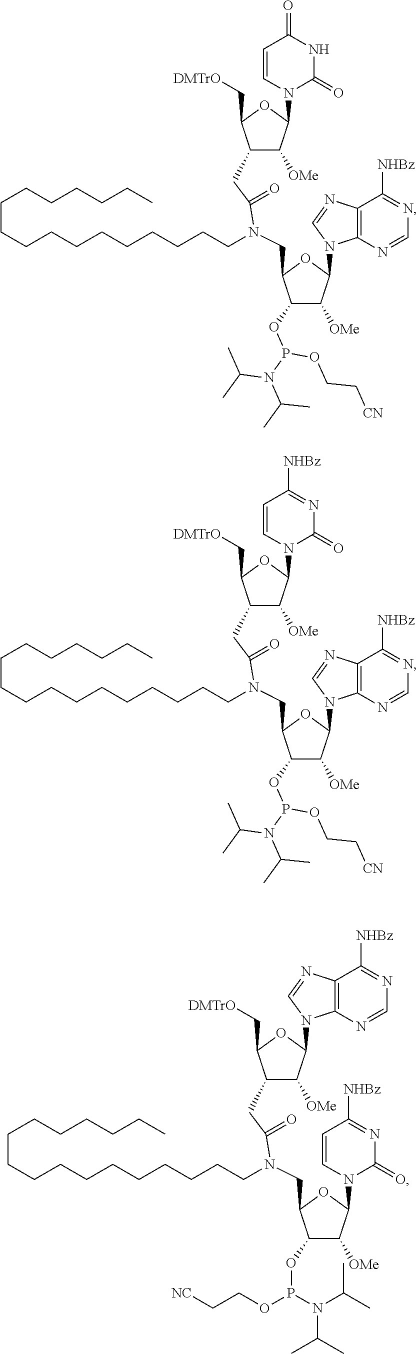 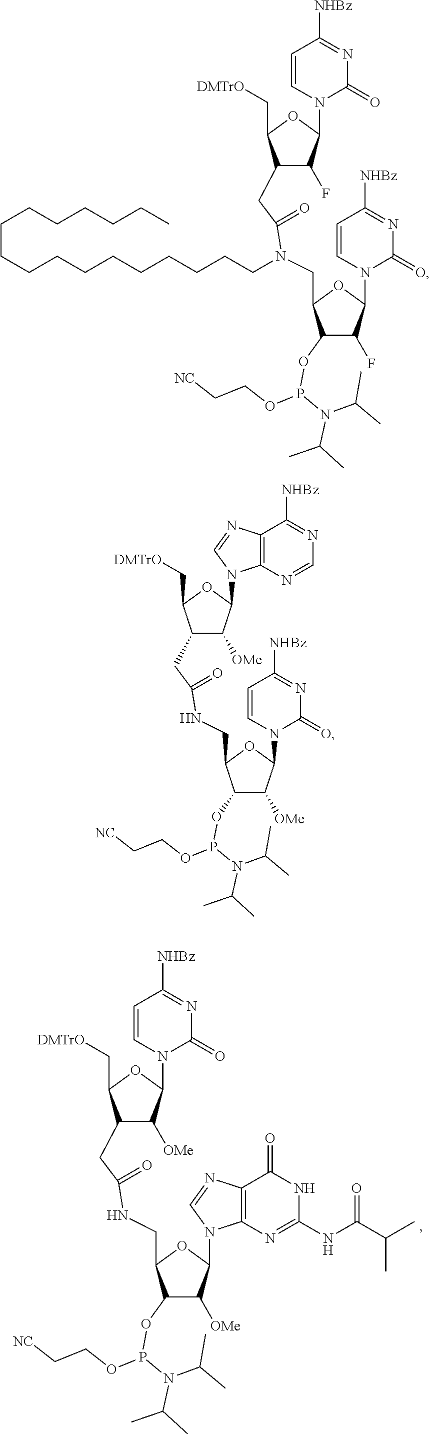 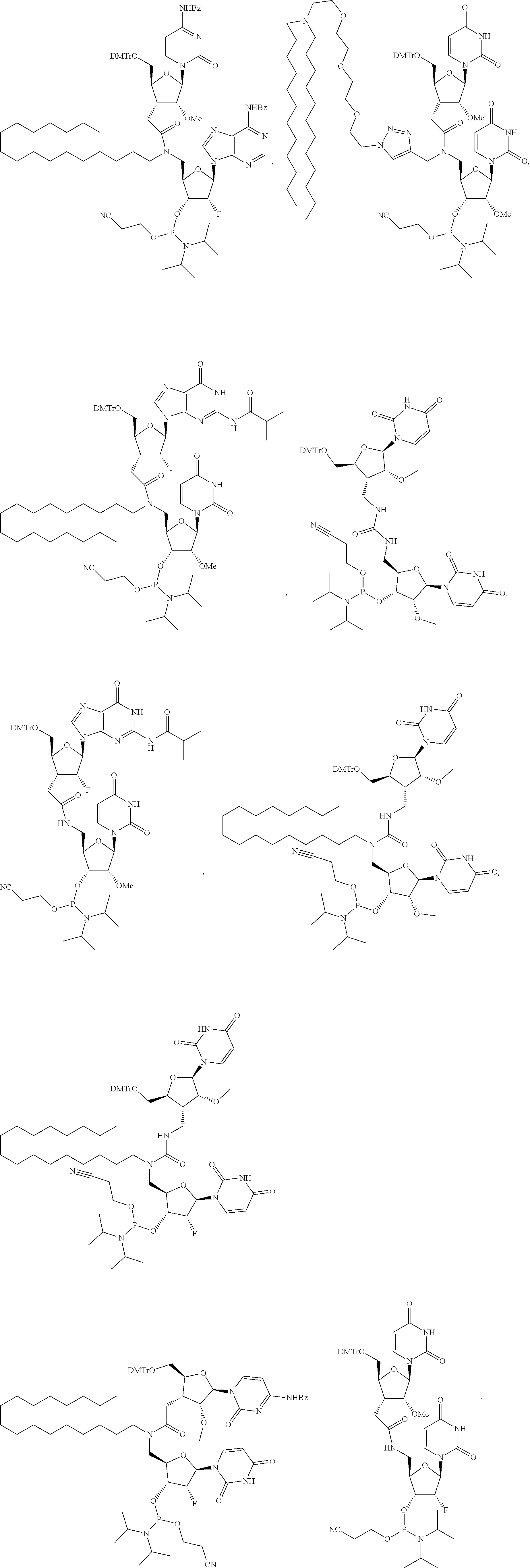 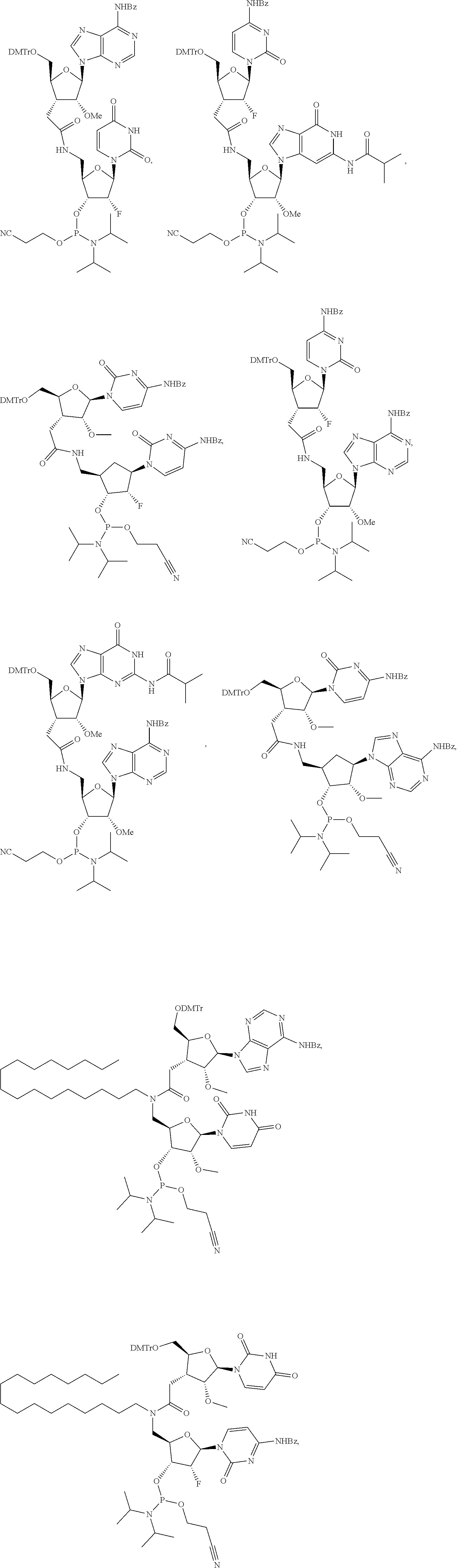 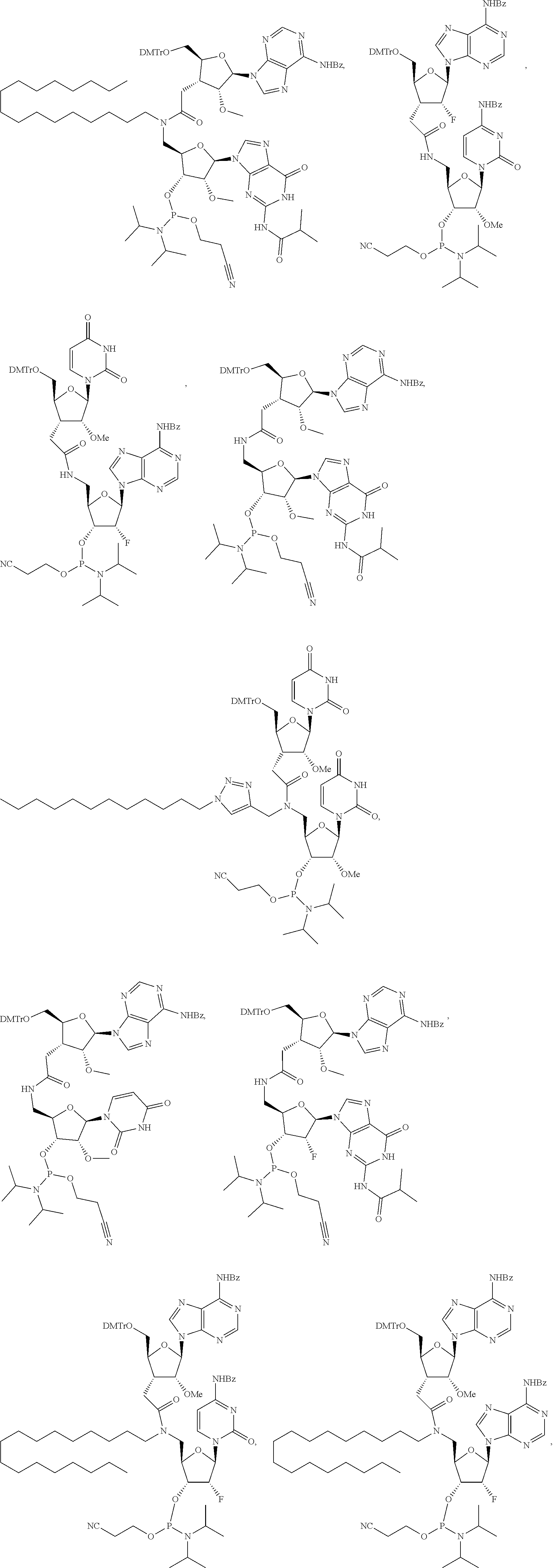 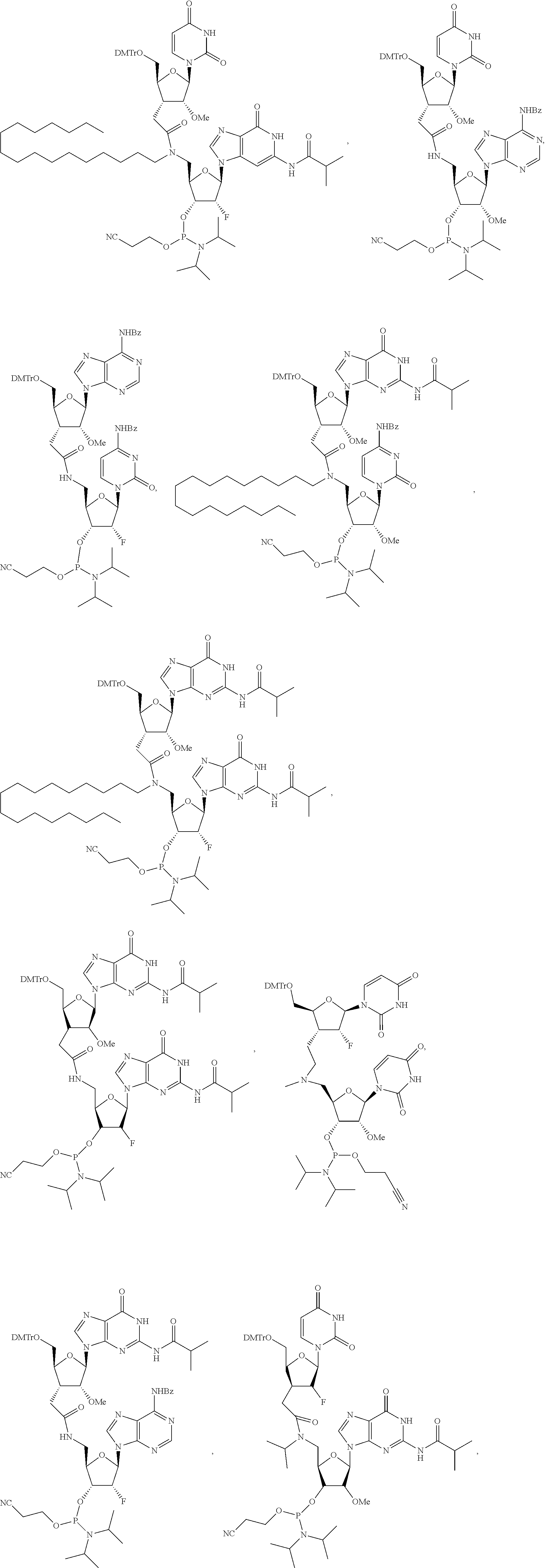 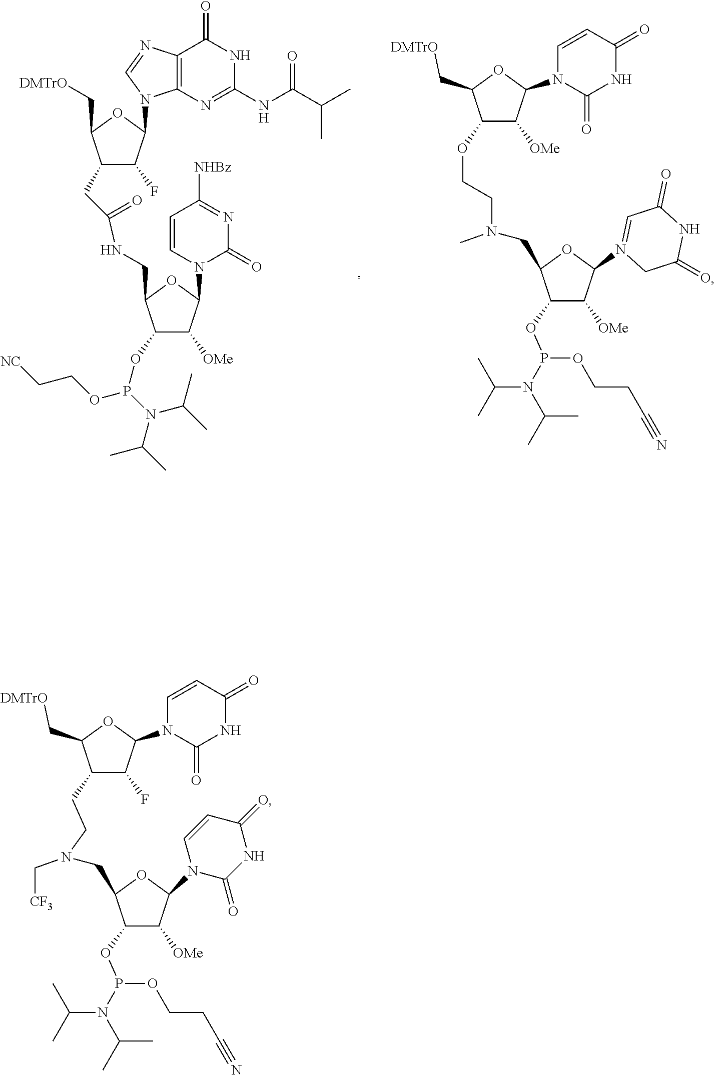 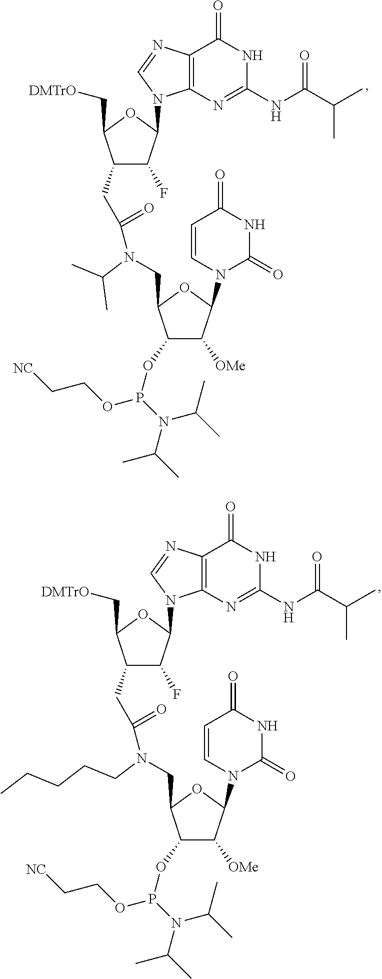 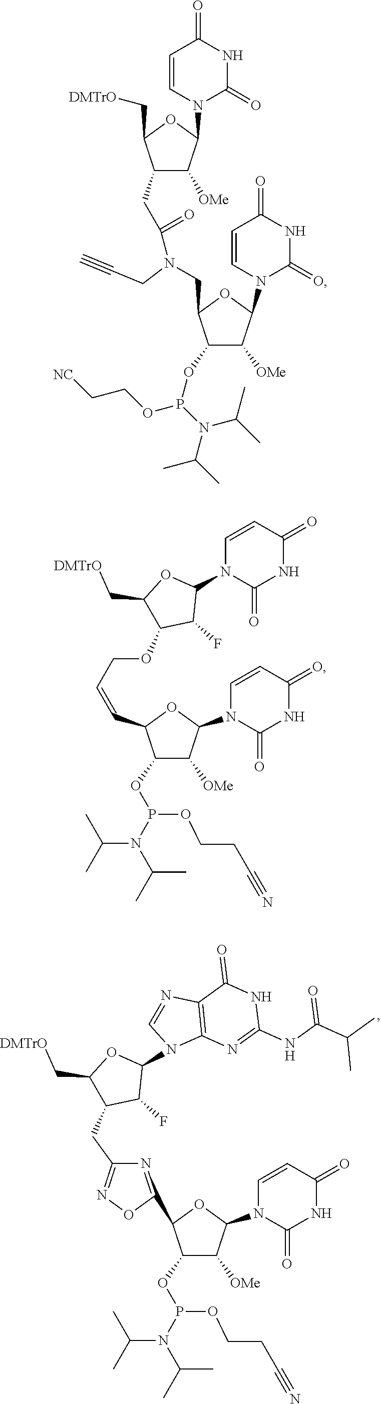 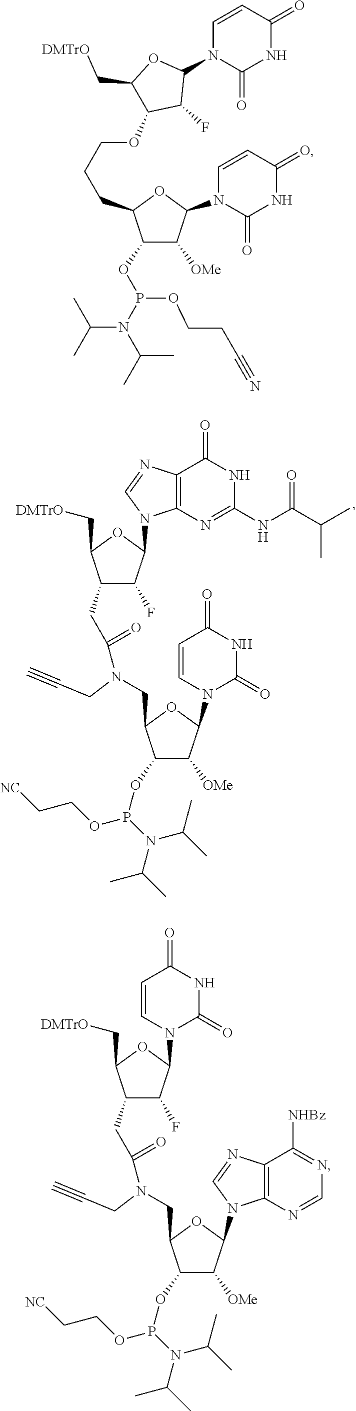 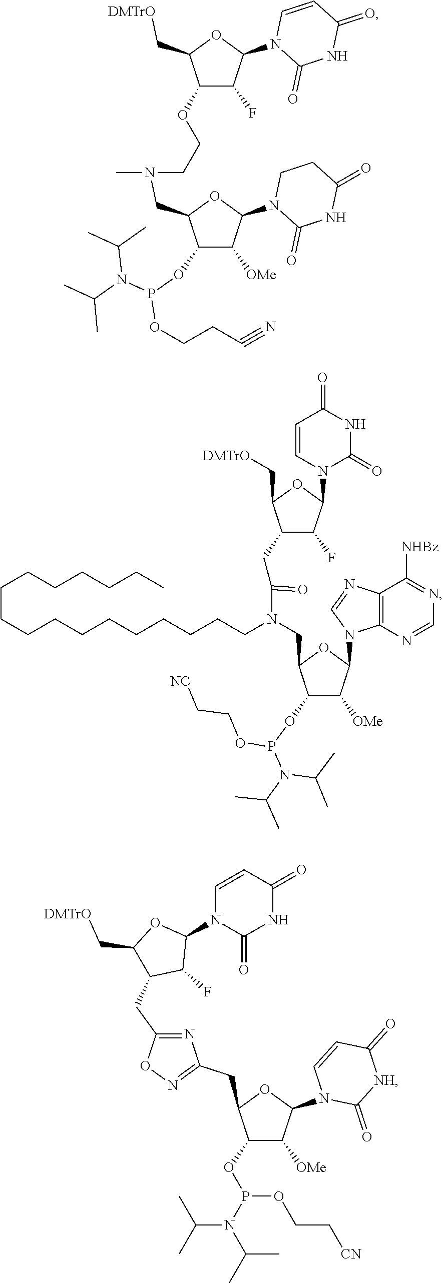 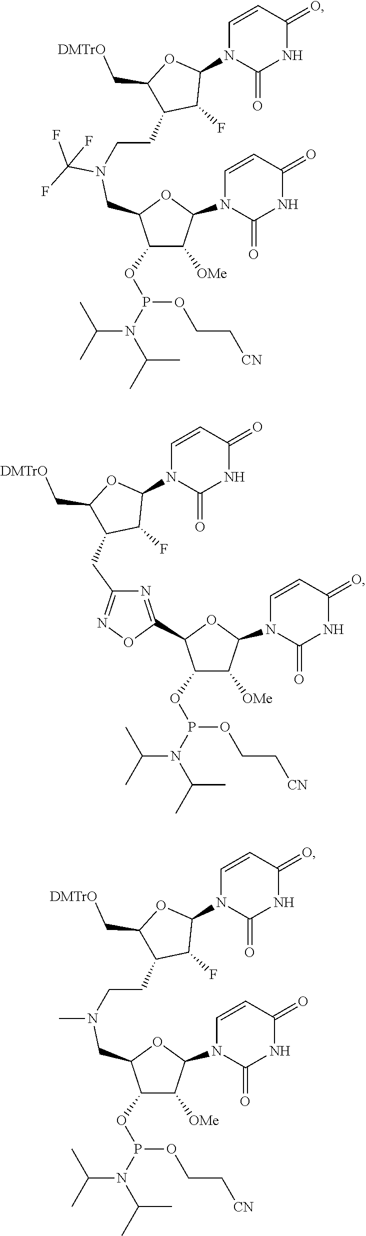 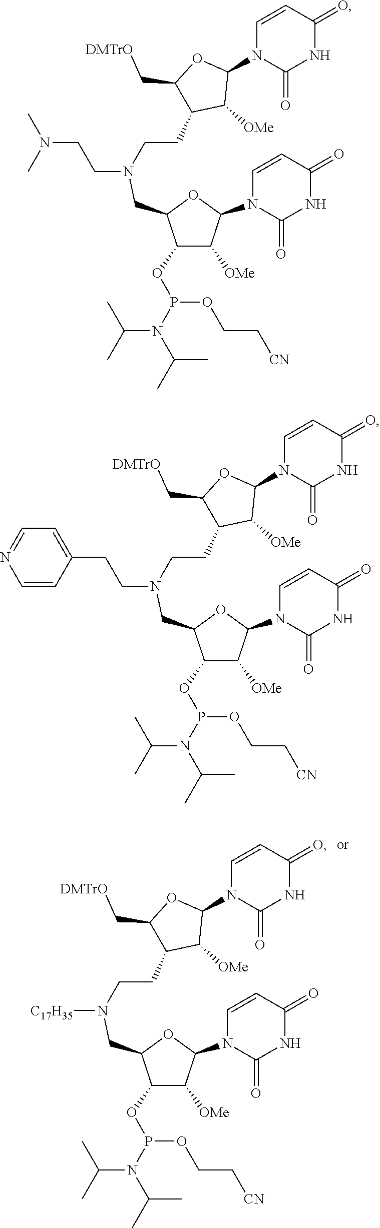 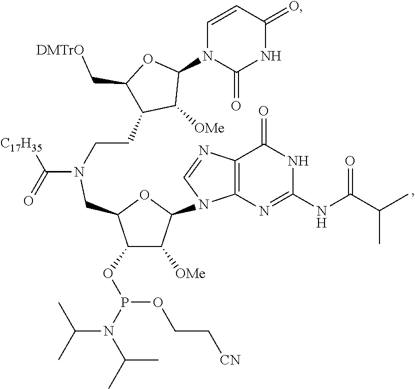
or a salt thereof.

143. A composition comprising a compound of any one of claims 1-142, and a pharmaceutically acceptable excipient.

144. A method for delivering a therapeutic oligonucleotide to a subject in need thereof, comprising:
administering a compound of any one of claims 1-142, or a composition of claim 136, to the subject.

145. A method for delivering a therapeutic oligonucleotide to the brain of a subject in need thereof, comprising:
administering a compound of any one of claims 1-142, or a composition of claim 136, to the subject.

146. A method for treating or ameliorating a disease, disorder, or symptom thereof in a subject in need thereof, comprising:
administering a compound of any one of claims 1-142, or a composition of claim 136, to the subject.

147. The method of claim 146, wherein the disease, disorder, or symptom thereof is a central nervous system (CNS) disease, disorder, or symptom thereof.

148. The method of claim 146 or 147, wherein the disease, disorder, or symptom thereof is Alzheimer's disease, or a symptom thereof.

149. The method of any one of claims 146-148, wherein the compound is administered to the subject intrathecally.
